SEQUENCE LISTING (290 sequences)

SEQ ID NO: 1 - DNA, 1131 bp, Arabidopsis thaliana
  source 1..1131: mol_type = genomic DNA
atgtcggcggagagttgtttcggaagctcgggtgatcaaagcagcagcaaaggagtggctactcatggtggtagctatgttcagtataatgtctatggcaatctcttcgaagtttctagaaagtatgttcctcctcttcgtcctatcggtagaggcgcttatggaattgtctgtgctgctacaaactcagagactggagaggaggtagctatcaagaagattggtaatgcttttgacaatattatcgatgctaaaaggactttgagagagattaagcttctcaagcatatggatcatgaaaatgtgattgctgtaaaggatataataaaaccaccgcagagagagaacttcaatgatgtttacattgtttatgagcttatggacactgatctccaccaaattattcgctctaaccaacccttaactgatgatcattgtcggttttttctgtatcagttgttgcgtgggctcaaatatgttcattcagctaatgtattgcatcgagatttgaaacctagcaatttgctcctgaatgcaaattgtgatctaaagcttggggatttcgggcttgcgaggaccaaatccgagactgactttatgactgaatatgttgttacacgttggtatcgagctccagagctgctacttaattgctctgaatacacagcagcaattgatatttggtctgtcggttgtatactcggtgaaacaatgacacgagagcccttgtttcccggtaaagattatgttcatcagcttagactcatcactgagcttataggatctcctgatgactcaagcttgggattcttaagaagtgacaatgcaagaagatacgttagacagcttccacagtaccctagacagaactttgctgctagattcccaaacatgtcggctggtgcagtcgatttgctggagaaaatgcttgtttttgatccaagcagacgcatcacagttgatgaggcgttgtgccacccatatttggcgccgctgcatgatatcaacgaagaaccggtatgtgtgaggccgttcaattttgatttcgagcaacctactttgacagaagagaacattaaggagcttatataccgtgaaacagtcaagttcaatcctcaagactcagtgtga

SEQ ID NO: 2 - DNA, 1695 bp, Arabidopsis thaliana
  source 1..1695: mol_type = genomic DNA
atggaagccctcctcctccctccttcgccggaaccccaaaatcaaatcaccaatccggcgaattcaaagccaaatcatcaatctggtgacgtacataaagatgagacgatgatgatgaagaagaagaaggatacgaatccatcgaatttggaaaagagaaaactcaagggaaagaagaaagagattatggacaacgacgaagcttcttcgtcctattgttctacatcttctacctctaattcaaattctactaaaagggttacgagagtggttcatagattacgaaaccctatgcggttaggtatggctcgacgaagcgttggtgaacgacaagctgaaaaattggcgaagcctctgggcttttcacttgccgcttttgctaatatggttattgcgagaaagaatgccgcaggtcagaatgtttatgttgatgatcttgttgagatctttgctactcttgtcgaagaatcattagccaatgtttatggtaataagcttggttcctttgcgaccaactttgagcaaacattcagcagtactctaaagatccttaaattgaccaatgaatgtgcaaatccacatcagtcaaacaataatgatggtgggagttgtaatttagatcgctctaccatagacggatgctcagacaccgagctatttgagagggagacttcatctgctacgtctgcttatgaagtgatgcaaggcagtgcaacagcaacctctttgatgaatgagcttgcccttttcgaagagactctacaactctcttgtgtccctcctagaagttcagcaatggctttgaccacagacgaaaggtttttaaaagagcaaacacgagcaaacgacctaaagaccgtggagattggtcttcaaataagagagttaaggtgcaaagagacggcgctaggattaaaatttgaatcaaacaacctggggaaagcggcgctagagttggatgtttcgaaagctgcattcagagcggagaaattcaaaaccgaattagaagatacaagaaaagaagagatggtcacaagaatcatggattggctcctcgtaagtgtcttcagcatgttggcttctatggtacttggcgtttacaatttttcaataaagagaatcgaggatgctacctcagtatgcgaccaatccgaggagaaaagttcgtcgtggtgggttcctaaacaagtttcatcgattaactcaggcttcaacaccttcatctgccgggttcgagtttgggtgcagatatttttcggtgtgttaatgatcattgtcttcacttactttctaaacaaacgatcatcaggtacgaagcagacaatgccgataagtttcatcgttcttttcctcggtatattttgcggtgtatcgggtaaattgtgtgtggacacattgggcggtgatggcaaactctggctaatagtttgggaagtgttttgccttttgcaattcgttgcaaatgtcttcacattggctttgtatggtctaatgttcggtcctataaacgtgactcaagagaccagatcgaaccgttgtaacagtatgtttccatattgggcaaggcgcagtgtcgtgtatgtggtgattctgtttgttcttccagtcataaacggtcttttgccatttgcaacatttggtgaatggagagacttcgctatgtatcaccttcatggtgggtctgactatgcttga

SEQ ID NO: 3 - DNA, 1122 bp, Glycine max
  source 1..1122: mol_type = genomic DNA
atgtctgctgttgagtcagctgaacacaacaacatcagaggagtaccaactcatggtggacgctatgttcagtacaatatctatggcaatctcttcgaagtttccagaaagtatgtccctcctattcgccctgtcggtagaggtgcttatggtattgtttgcgctgctgtaaatgcagagacaggcgaggaagttgccattaagaagattggcaatgcattcgacaacagaatagatgccaaaaggaccttacgggaaattaaacttcttcggcacatggatcatgcaaatattatgtccattaaagatattatacgtcctccacagaaggaaaacttcaatgatgtgtaccttgtttctgagttaatggacacagatctgcatcaaataattcgttccaatcagcaattgactgatgatcattgtcggtattttctgtatcaattgttacgagggctcaaatatgtacattcagcaaatgttttgcaccgagatctaaagcctagtaatttgctattgaatgccaattgtgaccttaagattgcggactttggtcttgctagaaccacatctgaaactgactttatgactgagtatgtggtcactagatggtaccgagctcccgaattgcttcttaattgttcagaatatacagcagccattgatatatggtctgttggttgcatacttggtgaaatcataacaagacaaccgctctttcctggcaaagattatgttcatcagctgagacttatcacagagctgataggttcgccagatgatgccagccttggatttctacgaagtgataatgctcgtagatacgtaaaacagcttccgcagtatccaaaacaaaacttttctgctagatttcccaccatgtctcctggtgcggttgacttgctagagaagatgctcatctttgatccaaacaggcgtattacagttgacgaggcgttgagccacccatacatgtcacctctccatgacatcaatgaggaacctgtttgcaccagacctttcagttttgactttgagcaaccatcattcactgaagaagacatcaaggaactcatctggagagaatctgtgaagttcaatcctgttccaccagtctactga

SEQ ID NO: 4 - DNA, 2613 bp, Glycine max
  source 1..2613: mol_type = genomic DNA
atgcgtgtcctctttctttttctgtttttccagtttctccattttcatttccccaaaaccctttcagccccaatctcagagtaccgtgcccttctctctctccgttcagccattaccgacgccaccccacctcttctcacttcgtggaactcctccaccccttactgttcctggctcggcgtcacctgcgacaaccgccgccacgtcacctccctagacctcaccggcctcgacctctccggccccctctccgccgacgtcgcccacctcccattcctctccaacctctccctcgcctcgaataagttctccggccccattcctccctcactctccgctctctccggcctccgcttcctcaacctctccaacaatgtcttcaacgaaaccttcccctcggagctctcgcgcctccagaacctcgaggtcctcgacctctacaacaacaacatgaccggcgtgcttcccctcgccgtcgcgcagatgcagaatcttcgtcatttgcatctcggcggcaacttcttctccggccagatcccgccggagtatggacgctggcagcgcctccagtacctcgccgtctccggcaacgagctcgaggggactatccctccggagatcggaaacttgtccagcctccgggagctctacatcggctactacaacacctacaccgggggcattccgccggagatcggaaatttgtcggagctggtgaggctcgacgccgcctactgtgggttgtccggcgagattccggcggcgctgggaaagcttcagaagctggacacgctgttccttcaggtgaatgcattgtcagggtctttgactcccgagctggggaacctgaagagcctgaaatccatggatttgtctaacaacatgctctccggtgagattccggcgagattcggcgagctgaagaatattactcttctgaatctgttcaggaacaagcttcacggagctataccagagtttataggggagcttccagcgttggaagttgtgcaactgtgggagaataacttcacaggtagcattccagagggtttgggcaaaaacgggagactcaaccttgttgatctttcttctaacaagttaactgggactttgcctacttatctctgttctgggaatactcttcagactctgataactcttgggaattttctttttggtccaattcctgagtcgcttggtagttgtgaatcccttacacggattagaatgggagagaactttttgaatggttccattccgagagggctttttggacttcccaaactaacacaggttgagcttcaggataattatctctctggagagtttcctgaggtgggttctgttgctgttaatcttggtcagattactctctctaacaaccagctttctggggttctacctccctccattggtaacttctccagcgtgcagaagctccttcttgatggcaacatgttcacgggtcggatacctccccagattgggaggttgcaacagctttctaagattgattttagtggcaacaagttctcgggtcctattgtgcctgagatcagtcagtgtaagctgttaactttccttgaccttagccgcaatgagctatctggagacatcccaaatgagataactggcatgaggatattgaattacttgaatctttctaggaatcatttagtgggtggcattccctcttcgatatcatctatgcaaagcttgacttctgttgatttttcatacaacaacctgtctggtttggtgcctggtaccggtcaattcagctacttcaattacacgtctttcttgggaaaccctgacctctgtggcccctatttgggtgcttgcaaggatggggttgccaatggcgcacaccaacctcatgttaaaggtctctcctcttcttttaagctgctacttgttgttgggttgctactatgttccattgcttttgctgtggctgcaatattcaaggcccggtcactgaagaaggccagtggggctcgtgcatggaagttgactgcgttccaacgtttggacttcactgtcgatgatgttttgcattgcttgaaggaggataatattatagggaaaggaggtgctggcattgtctacaaaggggctatgcctaatggggatcatgttgctgtgaaaaggcttccggctatgagtagaggctcttcacatgatcatggcttcaatgctgagattcaaacattggggcgaatccgacacaggcacattgttaggttgttgggcttctgttcaaatcatgagacaaaccttttggtctatgagtacatgcccaatggaagtttaggcgaggttcttcatggaaagaaagggggtcatttgcattgggatacaaggtataaaattgcggtggaggctgccaaggggctttgctatctgcaccatgattgttcgccactcattgtccatcgtgatgtgaagtcaaacaacatccttcttgattctaatcatgaagcccatgttgctgattttgggcttgctaagttcctgcaagattctgggacatctgaatgcatgtctgctattgctggttcatatggatacatagctccaggtaccgtccaatttcgacataattaa

SEQ ID NO: 5 - DNA, 2473 bp, Glycine max
  source 1..2473: mol_type = genomic DNA
agggtttaagcaaatacgggaactaggcggcaaagtacaatgaaggtaagattaaaatcgaagacacgcgacgtttcagaatataccaaaagtgaaaaaaggaaacactctctctcccgcgcgccctcgcttgtcaatgattggatcgattgcaatattaacgttgggcgcttgaaatttgagggcttttcatattaaatttcaatttcaatttcatggtctcttcactgaaattgcgacccaaatggctgagattcagaattgttcctcggaaccagaaccaaccgcaatcaacgaatgggagggtcaagcttcgatgaagagcacaacgcaaagttcagaagcctccgatacgacgtcgtcacgaaaagagaataaaggtaaaggtaaggctgcggcgttcaaacgacggaaccctagagttcttgttcgccgccatagggctaataatgtggacactattggccttcctcttggcatgtcattcgccgctgttatggctcaggtgatgtatagaagagatgtagcagccgagagtatgtctcccagtcatctttcgatgatgtgttcatcggctatcaaagaatctcttgccagtgtttttggagacaagctagatggtttgacgaggaactttgaacaatctttttgtagcaccttgagtactcttcagttaatttatgaatcatccaagagcaatgaaggaaataaattaaataatacaaagatggaaattatgagttctaaattgactctcaataaagaggaatgctcaggtgatattgttacagaggtcggtcattcaagacatgctgaaatccaagatcaatcaattagtcatgattctcctgaagaggtcagggataactttcatatagattcaattggtcgcgattctcctgaagaaggcagggataacattcataataattcagttagtcttgattctcctgaagagagcagggataactttcatatgggttcagttagtcgcgatctcactttgtatgggcaatcaaaccaaatggtttccttttctcaaatatcttttggatctgtaaataatcctatggttagtatttttgagaagtccatcatggagcagtgtcgttctaatgacctcaagacacttgaacttggccttaaaatgaaggaattgaaattgaaagaggatgaattggctctcaaccttgattcgaataatttaaacagatcaaaattagtcatgggagaatcaaagcaatctttcaaagaggaaaagttcaagacccagttagaagatacgaggcatggtgaactcaaaaagaagtgcattgactgtcttattactggtttgcttatcatgtcatcctcacttttgtatggtgcctatgtttattcctacgagcggattactaaagctactgaatcatgtacaccatcaacccaggaatcttcctcctggtggactcccaagtcaatggtcttattcaattcaaagttgcatattttgtggtgtcaagttcaagttatgagccgaatggcatttggcatcttgatgatttttgctgttgcatatttgctcttgcagcggtcgaccacaacatcacagactatgccagtcactttcatccttttaatgttgggaattttttgtggctattgtggcaagctatgtgtagagacattgggtgggagtggtattgtgtggctcttatactgggagattatgtgcttgctgcatttcttatctctttgctggacatccgctttgttccggatccttcatggaccagtcactccatcacaaacaatggaggagaatacaattcttcggtattggattcgcagagttctgttctatgctgctatgcttgtgtttctaccgttgttttgtggtctcatgccatttgctagcctaggtcaatggaaagaacattttacattgaaggggtcagattttaatggatccgaatggtaaagcactgctcggtcatggaagacctttatgtataacaactctatgccatactatgggatgcttagcttgtggttagacaactagccagtagccaagcaccatcggtttgtattttgcccacagctagttctatttagttcatgttttgctaattattttctgctatgatgtgatatttaaagtaatgcaatgactattgtattgtattgtgagcaccaccttcctttgcatttgccgctggattcttcttttatgatttaagactctgtaggtgagtccttaaagaaggaaatgtaatttttaggggtatgtatttagttagaatgattagctgtcttaatgtatttttcattggcagttaattatgaattatattttgaaaactttatttttttataattatttttaaaagttataccaacatggtgtgacatgattaatagaggatcttgttccagaagtatacgatcatgagtttgattcctgacttgtttgtatgaaaaaatatcgatt

SEQ ID NO: 6 - DNA, 2028 bp, Glycine max
  source 1..2028: mol_type = genomic DNA
atggcttattcagccgaaccctcatcatctttgagctttacctcatcttcccatctatcaaatgggtcggttagtcacaacatatgctcttcttatggctctgaccctggacctaaccttgaggctctcagtttgagtaagcttagctccaattttgagcagcttttgattgaaactgattgtgattatagtgatgctgacatcgttgtggagggaatttcggttagtgttcatcgatgtattctggcctctaggagtaagtttttccatgaattattcaagagagagaagggttcatcagaaaaagaagggaaattgaagtataacatgagtgatttgttgccttatggcaaggttggatatgaagccttcctcatattccttggctatgtatatactggtaaactcaagccctctccaatggaagtgtctacttgtgttgacagtgtgtgtgcccatgatgcttgtagacctgccattaactttgctgtggagttgatgtatgcctcttacatttttcaaataccagagtttgtatcacttttccagagacgtctacttaactttatagggaaggctcttgtggaagatgtcatcccaatcctcactgttgctttccattgtcaattgagtcaacttgtcaatcaatgcattgatagggtggccagatcagaccttgaccagatttcaattgaccaagagcttccaaatgaactctcacaaaaagttaaattgctccgccgcaaccctcagcgagatgttgaaaacgatgcttctatagtggatgctttgtctctaaaaagaatcactagaatacacaaggcattggattcagatgatgttgagcttgttaaacttcttttaaatgaatcagacattactttagatgaagccaatgctctccattatgctgcagcctactgtgatcccaaggttgtttctgaggtacttggtttgggactcgctaatgtcaatcttcgaaattctagggggtacacagtgcttcacattgctgccatgcgtaaagagccttccattatagtatccctacttacgaaaggggcttgtgcatcagatttgacttttgatggtcagagtgctgttagtatttgtaggaggttgacaaggccaaaggattatcatgcaaaaacagagcaggggaaagaaacaaacaaagatcggatatgcatcgatgttcttgaaagagaaatgtggaggaatccattggctggggatgcctgtatgtcttcccataccatggctgatgatctccacatgaaactactataccttgagaacagagtggcatttgcaagacttttcttcccttcagaagccaaactagccatggacattgcacatgctgagacaacatctgagtttgctggtctttctgcatcaaactcaaaaggttcaaatggaaacttaagggaggttgatctcaatgaaactcctatagttcaaagtaaaagacttttttctagaatggaagcccttatgaaaacagttttgaattctggcaccgactatgttctatgttgtggttgtggcagtggagatggggcggcgctacttcccgcattgctcggaagtgttggataagttcatggaggatgacttgcctgacttgttttaccttgaaaagggtactaatgaagagcagagaatcaaaaggacacgtttcatggagcttaaagatgacgtccacaaggctttcaacatggacaaggccgagtttagccgctctgggatttcatcttcatcatcctcatcatccctcagagattctgttgtacattacaaggctaggaaagtgtaataataaaagcatacatagcgttttttattttgacatacatattatatatagtgcaaatttgaaaggtgaagcctaccaactcatctgtaaaattgtgaaagcaggaaagttgatgttatagtaccttttgtaatcaacttgctcatgtattttgaaaatattttcatatgtttggtggagaaatttacggcattttttttgaagataaa

SEQ ID NO: 7 - DNA, 1504 bp, Glycine max
  source 1..1504: mol_type = genomic DNA
gatatctaggagggtgaaatctgaatgaatataatgcgaactaggttttttttaggatgaaaaaaaaatatctgtaatttgaaaaaaaaaattatagtaaatggaagtgtaatttactcaaatggaaaatgtgtttggcgccagtaggcactgccctctgctacttgtgagcggtaaatgcaaatttcagttctcattctcaacaacaacgcactgaacgaagatagaaaagagttttagcatcgtgattcgtgacctatcaatggcgcagacgacccatcccatggacatcgacgaccacacttacaccgccagtcctgacaagggcaaaagtgtcgtcttcgccggaaaccctcccgccgccggcaaggccattccgtgggtcgaaaaataccgccctcagtccctcgacgacgtcgctgctcaccgtgacattgtcgatacaattgataggctaaccactgagaacagattgccccatcttctgttgtatggccctcctggcacgggcaaaacatcaacgattcttgctgtggcacgcaaactgtatggttcacagtaccaaaatatgattttggagctcaatgcatccgatgatagaggaattgatgtcgtgcgccagcagattcaagattttgctagcactcagagcttatcatttggtgttaagtcctctgtaaaattggtattgttagatgaagcagatgccatgacaaaggatgcccaatttgcgctacgtagagtgattgagaaatatacaaaaagcactaggtttgcacttatctgtaatcatgtaaacaagattattcccgcacttcaatcaagatgcactcggtttcggtttgcccctcttgatgctgtccatgtcactgaaagactgaaacatgttattaaggccgaagggcttgatgttgaggacagtggcttagcagcttttgttcggcttagcaatggtgatatgagaaaggctttgaatattcttcagtcaacacatatggcttctcagcagataacagaagaagctgtttacctgtgcactggaaatccattgccaaaagacattgagctaatatcttactggcttctaaatgaacaatttgcagatagctttaaaagaatagatgaaatgaagacaaggaaagggttggccctgattgatattgtcagagaagtgacaatgtttgtttttaagattaagatgccttcagctgtgcgagtacagttgatgaatgatttggcagatatagagtacagattaagttttgggtgcaacgataagcttcagcttggatcggttattgcttcttttacacgtgctagatctgcactggttgcagctgctacatagcctacttctgaaaaattactttcacatactactagggcctggcttgtttgatttcctgggcatgtcactacggataatgggattgggaggatcttaatttgtgtaaagatagtaactgtgtcaatcttttttaacttaagatgagttttaaagttt

SEQ ID NO: 8 - DNA, 2399 bp, Glycine max
  source 1..2399: mol_type = genomic DNA
tgtatttctttttgagtcttggctttatgtgtgcatatactatacatataggctcggattggttaaattagattcatactacttagaggagatatatgatgtttgttattttactcatatatacatatgatatctttggctgataaaaaaatgtgtgttattctatcttttgtaatgttttataacttgtgttaaataaaatggatgaaagtgtttttttgaaagttgtaaacttaatataattaaaatattttggtaatgtttaccaaattatgacattgtcacccctatccataattaagcacccggcctaagtaacttcaaactatatttcactcataatttagtaatgtttaccaaattttggcgggaagctaagcaactcctttattgttttttttactaaatttttaattacagttttattaaatttgtttggtttgtgtctatcaaattctcagcgtcagcgttcctgtgtgaaaattaagtaattacatctcaatttttgtttcgaagaaacacaaaaataagcaaaagtgggagcgaagaatggagaaccccagcagtagtgaaaggagcagagcaggaatcgaagataacatgctggccatgtttgatgcctccgaaaccaaagatgctcaccaagatgccctagatgaccggattgcttttattgatgctgttcgtgcttcttgtgaacatggaaaaccgcccacttcaaaaatttttggggcggtttttcacatgctgaggacagggaagtctctagagttgattgtgggaagttataagcttctggttgatttagacaagcacttccctcgcgtgtatttatctggcacggatgattcccgatcatcatctaactctccatcaaaattggttgtggttaaagaggcatggtctcccattatcgattttgtggataaggccactgctgttagtgaagctggtgataaacagtctggtggatctcttgacccctctagctttcaagtcttgattgaagggctggctgaagttttagcagagaccaaatttgaagcagcaagcatggagcccttgaggaacatgttaatctttcagtaccttgtcgttgtttttgaggatgattttttacctcgcaatgcaaccttgaactggagcatgcagagggagtctttgctcagcctgctactgggatctaggaaaataaattacaaaagcttgatgaaggattgcatggcaattatgtgtcaactatctcaacttctaagtgaactcagtaaacatccagtgcttcaagaaagttctgaatctaagttatccaaaagttgtcatactgcattatctcttgctttacttggaattttaaaggacacatgtgtctcaatggagaaacttctagtgatgattatggaccttgatatggcaaggaagaaagcagatattgaaggtcgcactacaagagcagacagtccaagaacacctttgatggacataattctggatgaactagcttacaacaaagatagtgtccctgtttttcttaagatggtcagtgaacctaaatggaagctagaaatagttgtgcagtatctgtggaaatacataaccaagccttctgttcgcacccgcaagtcaaatggtcatactgaagatgcaacatttggtggggctttgaagtgcttttcaaatataactggcaccaagagcttaataaaaaaaattggtgtagatgtagttcaattccttctggctcatggttttcaggctcacttgtcaatattgtcaaaaggaaatgctggtgacaaacaagggggaaacagtgctcttgtggatttgtgccaggcttttatctccgcttttgatagtttgagaagcacaaatgagcatatggagatattgtctatgggaaaagaggcactatttactgccgcaactattatctctgtgaaatcataggagtaatcgaagactttaatacatgagatatactttgcctcttctctgactgaatgataatatcccaaaccattatagtcattttatttagaggctatgccgcagcactgtatagaaaagtacagcctccttgcagctttatagtcgtagcttagatgctatgctgggtttccaactcctagtgatgcatcgtcttgaaggaggtttaattttgacaattgttacagatctgacaaaacactgcaagatgcaattagatttgataccaccttggaattttttttctcttgttcacaatgcagcattgttgaactctagatttacattgatgaatgaaattctccctcagaatgggcagtaaacttagtccaatttgacactatgatacccctatttcaaacccgatcttcaaaaattaatatcctacaaactattgcactaacattttctaaaca

SEQ ID NO: 9 - DNA, 4664 bp, Glycine max
  source 1..4664: mol_type = genomic DNA
aaaacaaaacacttcaacctttctcgggggagagagacagttatccattgttttcatctcctttttcttttctctctcattcagttatcgatctgactctctctctctctctctctatttatttgtcattttggtgaagtggggtgtttggttttgtcatttgggaggtataaagtttggagatgtttgtgttatttggcttcattgcgtgaaaattcatctggggttgttcggattctctttgatctgtctgtttgcagcggaaactggtagaaaatattggttttttgaggtggtttttgtgtgcatatgaagttttatcgcatggtctttgtttttgaagtagatagtggatatctgttcactgttttgatcatgcgagaatctcaaagaaactgaactttttctttgttcttttttttttttttattgttgttggtaatgggaaaagtgctttgttttctcagccatgtacacaaaggagacttcattatttagaatctgtcacccttttcattttctcttagccttttctcatctacgtaacaaaatttgagatcagaagttctttttcttgtttccactttgcaaactgcttagaatggttgttaaaaactattctttttgtgtgtatgattatagcttcaatttgagatcacaagtttatgttttttgtcttcactttgcatactgttgagaatgcttgttaaaaactagaattttttattttcttttgtgataatagcttcattatctacagattaagaataaagggagtcttccaaattgatcttctcagctatcagaaagtgttgaaagtgatgagtgtggcttgaactactactactactattactaccaccttttgatcccttgttgagaagagttaagtagagagggtcctatggagtcggaaggggaagctggggcaaaaccagtgacagcattgggtgcccaagtgtgccagatttgtagtgatggtgttgggaagactgtggatggtgaaccgttcgttgcgtgcgacgtttgtgctttccctgtctgcaggccttgctatgagtatgaaaggaaggatgggaatcagtcttgcccccagtgcaaaacccggtacaagaggcacaagggaagtcctgcaattcttggagacatggaagaggatggtgctcctgctgctgatggtgccagcgacttcaattatgattcagaaaatcaaaatcaaaaccaaaaccaaaagcagaagatttcagagcgcatgttgagctggcaattgacatactcacgaggagaggaggttggtgctccaaattatgataaggatgtttctcacaaccacattcctctactgaccagtggacaagaggtatctggagagttgtctgcagcctcacccgagaggctctcaatggcatctcctgcagttggtggcggaaagcgtgtccataatattccatattcatctgacattaatcaatctccaaatatcagggccggggatccaggattgggtaatgttgcatggaaagaaagagttgatggctggaagatgaagcaagaaaagaatgttgttccaatgagcacaggcctggcggcttctgaaagaggagctggagatgttgatgctagtactgatgtgcttgtggatgattccttgttgaatgatgaagctcgacagcctctttccaggaaggtttctattccatcatctaggataaatccatatcgtatggtcattatgctgcggctggttatcctttgcattttcttgcattatcggataacgaatccagtgcccaatgcctatccattgtggttggtatcagttatatgtgagatttggtttgccatatcttggatattggatcaattccccaagtggcttcctgtaaaccgtgaaacatatctcgacaggcttgcactaaggtatgatcaagaaggagaaccatcgcaactagctgctgttgacattttcgtcagtactgttgacccattaaaggagcccccacttgtgactgccaataccgtactatctattcttgcagttgactatccagtggataaggtctcctgctatgtgtcagatgatggtgctgctatgttgacatttgaagcacttgctgagacatcagaatttgcaaggaaatgggttccttttagcaagaagtatagcattgaaccccgtgcacctgagtggtattttgctcaaaagattgactacttgaaagataaggttcatccatcatttgtcaaggatcgtagagcaatgaagagagaatatgaggagtttaaagttcgtgttaatggacttgttgcaaaggcacagaaagttcctgaagaaggatgggtgatgcaagatggtaccccgtggcctggaaataacatcagagaccatccaggaatgattcaggttttcttgggccaaagtggaggacttgacactgagggtaatgaacttccacgtttagtctatgtttctcgtgaaaagcgtccagggttccaacatcataagaaagctggtgccatgaatgcacttgttcgagtatctgcagtccttaccaatggacctttcttattgaatcttgattgtgatcactacataaacaacagcaaggccttgagggaagctatgtgctttatgatggatccaaaccttggtaaacatgtttgctatgtccagtttccacagaggtttgatggtattgataggaatgatcgatatgccaatcgtaatacagttttctttgatataaacttgagaggtttggatggcattcaaggtcctgtttatgtgggtactggatgtgtcttcaataggacagctttatatggttatgaacctcctcttaaacccaagcacaaaaagcctgggttgttatcatcactctgtggtggaaatcggaagaagagttcaaaatctagcaagaaaggaacagacaagaaaaaatctaacaagcatgttgacccaactgtgcctatttttaatctagaggatatagaagaaggagtagaaggtactggatttgatgatgaaaaatcactacttatgtcacaaatgagtcttgagaaaaggtttggtcagtctgctgtctttgttgcctctacgcttatggagaatggtggtgttcctcagtctgctactccagaaactcttcttaaggaggctattcatgttatcagctgtggttatgaggataaaactgattggggaagtgagataggatggatttatggttctgtcacagaagatattcttaccggatttaagatgcatgcccgcggttggcggtctatatactgcatgcctaagcgcccagcgtttaaaggttctgctcccatcaatctttcagatcgtctaaaccaagtgcttcgatgggctttaggttccgtggaaattctctttagccggcactgtcccatctggtatggttatggtggaaggttgaagtggcttgaaaggtttgcatatgtgaacaccacaatctatccagtcactgccattcccctactcatttattgtatcttgcctgctgtctgtttgctgactaacaagttcattattcctcagatcagtaaccttgcaagcatatggttcatctccctcttcctttccatctttgctactggaatcctggagatgaggtggagtggcgttggaatcgacgagtggtggaggaacgaacagttttgggtcattggtggtgtctcggcgcatctctttgctgtcttccaaggcctgctcaaagtgcttgctggaattgacaccaactttactgtcacctccaaggcctcggacgaagatggagactttgccgagctctacatgttcaaatggaccacccttctcatcccccccacaacccttctcataataaacatgattggggtggttgccggcatctcctatgccatcaatagtggctaccaatcttggggacccctctttggtaagcttttctttgcattttgggtgatcatccatctataccctttcctcaaaggtctcatggggcgccaaaacagaacaccgaccattgtggtggtgtggtccattcttcttgcatccattttctcactcttgtgggttagaattgatccattcaccacaagggtcactggtcctgatgttgaggagtgtggcattaactgctagaagaccaaaagacaggtttaatgtattgtgttttgggttgtaattatagttattgttttgtagctgttatattttggtcatgtgtcttttaagttatatgtgcagtttatagaagatgcataagaaagcaaagtagaactaaactttgtggcattgttttatttgtaatactaatactaacgtgcctccagttttgtggtgtagctatgtattataagctaattcggaggctcacattttgtatgtatacatctcagtgagtgtttcatgtgatcagaaaaccgaggacttttgcatttatttttacaccttattggtctactattaaattctgaaaacaccgagcttatgaatcaatcaattggaataaggttggttttgcaatttagtgtattagttttgagtttgaattttgagaatgttgtcacattaaatacttgattcgagatttttgttgcttgtagagaatttatttatctcgagtatgattgcttcatgtagaatatacttgtgtcgt

SEQ ID NO: 10 - DNA, 2114 bp, Glycine max
  source 1..2114: mol_type = genomic DNA
ggtcctaccatttcctcactcacacactcatcatcatcataacccattcccctttcgccacttgaaccaaaacctcttccacctttatcttttccttcactcttcgcttcaatattatgacggaggatcggaacgtgcggaagacacgtgtggtcgacctggtcctcgactgtgtcatcccttacatcgacgaccccaaggaccgcgacgccgtctcacaggtctgccgacgctggtacgaactcgactccctcactcggaagcacgtcaccatcgcgctctgctacaccaccacgccggcgcgcctccgccgccgcttcccgcaccttgagtcgctcaagctcaagggcaagccccgagcagcaatgttcaacttgatacccgaggattggggaggccacgtcaccccatgggtcaaggagatttctcagtacttcgattgcctcaagagtctccacttccgccgtatgattgtcaaagattccgatcttcggaatctcgctcgtgaccgcggccacgtgcttcactctctcaagcttgacaagtgctccggtttcaccaccgatggtcttttccatatcggtcgcttttgcaagagtttaagagtcttgtttttggaggaaagctcaattgttgagaaggacggagaatggttacacgagcttgctttgaataatacagttcttgagactctcaatttttacttgacagatattgctgttgtgaagattcaggaccttgaacttttagctaaaaattgccccaacttagtgtctgtgaaacttactgacagtgaaatactggatcttgtgaacttctttaagcatgcctctgcactggaagagttttgtggaggcacctacaatgaagaaccagaaaaatactctgctatatcattaccagcaaagttatgtcgattgggtttaacatatattggaaagaatgagttgcccatagtgttcatgtttgcagccgtactaaaaaaattggatctcctctatgcaatgctagacacggaggatcattgcatgttaatccaaaagtgtccaaatctggaagtccttgagacaaggaatgtaattggagacagagggttagaggttcttggtcgttgttgtaagaggctaaaaaggcttaggattgaaaggggtgatgatgatcaaggaatggaggatgaagaaggtactgtgtcccatagagggctaatcgccttgtcacagggctgttcagagcttgaatacatggctgtttatgtgtcggatattacaaatgcatctctggaacatatcggaactcacttgaagaacctctgcgattttcgccttgtgttgcttgaccacgaagagaaaataactgatttgccacttgacaatggggtgagggctctactgaggggctgtaacaagctgaggagatttgctctatatctcaggcgtggcgggttgaccgatgtaggtcttggttacattggacagtacagtccaaatgtgagatggatgctgcttggttatgtgggggagtctgatgcagggcttttggaattctctaaagggtgtcctagtcttcagaaactagaaatgagagggtgttcatttttcagtgaacgtgcacttgctgtggctgcaacacaattgacttctcttaggtacttgtgggtgcaagggtatggtgtatctccatctggacgtgatcttttggcaatggctcgccccttttggaacattgagttaattccttctagaaaggtggctatgaataccaattcagatgagacggtagttgttgagcatcctgctcatattcttgcatattattctcttgcagggcagagatcagattttccagatactgttgtgcctttggacactgccacatgcgttgacacctagaggccagagctgtgtatataccatttttcttctcccctttcatatgttgtttctatgtttctctgctctatttctagttcattttagacaaattagtcttgtaataagcaagtgttttcatttgtaattctgaaacactacttcccctaacgcaattggctcccttcaaaactcaacattctcaattttgtaaataaaaagcaagagttgcccccccccccgcgc

SEQ ID NO: 11 - DNA, 2793 bp, Glycine max
  source 1..2793: mol_type = genomic DNA
gcgtgggattttatgctctctcagtcccttccactgaagataaggtgtgacttctcttcttcttgctgagtttcaaaatctccatttttatccgctaatgggttctctctctatcaattttaggaattttatttttttggaaggcaaatattttttaggacttgttgttaatgacatatatcatatttaagttgaaacacagttttgttttttgtttttgtttttgtttttttgaaactgtgaatgagttttggaacggcttattgagtttgtattttgcatctgccactaatttccaatacattgcatacagattttgtatagtttatttgctattctctatttattatgttgaaaggtgttgcaattcatgtctgctgctcctgtaggaacaagaacaaccccttttgtgagttgttgattggatttattaggagggaactgagaaagattgattttttcattgagttgcatagtcaaatctatttgattgaaattgtttctttgggtaagctatggaagggttactgaattctttgcataaccaaagttgcttaaaaccctttggtttcactcccagacttggttataccttccccgtgatcacaagaactgggcatttaaatcgaaaacccgaatcctgcaccttgagaagtaactgtgcacaattaggtagagacaccagcactagctctagaggggctattgttgctgcattgtatagtaatgataaaggagagggttctaatacagactgggaatcggagttcctcggggaacttgatccgtttggttaccgagctcctaagaaaagggaaaaagaaaagaggtcaatgttgcttgaagccactgacggaatggattggtgtgtgagggcgagaaaggaagcactgaaatcaattgaagcaaggggcatggctcatctgatggaaaatatggttactgttaagaagaaaaagaagaaggataagaaaaagttggaaagcaagaagaaaatagttaagaaaattgagaaaatagaagatcttgactttagtttggaggaagacttgccacagcctatggaaactgagattgatgttggtgatttgaaaaggagagtgagtatatttaatgatggaatgtttatagaaaagaaggagaaaacaaaggaagaatttgtcaataggctttctcagttctctgggccatctgatcatagaaaagaaatcaacttgaacaaagcaattacagaagcacagactgcagatgatgtgttagaggttacttatgaaacaattgttgcagttgcaaaaggccttagtccctcacctctatccccattgaacattgccactgctcttcatcgcattgctaagaatatggagaaggtttctatgatgagaactcgaagattggcttttgctcgacaaagagagatgtctatgcttgtgagcattgccatgacagccttacctgaatgctcagctcaaggagtctctaacatatcttgggcattgtccaaaattggaggcgagctgctttatctttctgaaatggatagaattgcagaggttgcactgaccaaggttggggaattcaactctcaaaacattgctaatattgcaggtgcctttgctgcaatgcagcattctgctcctgatcttttctcagagttttcaaaaagggcttcagacataattcacactttccaagaacaagaacttgctcagcttttgtgggcttttgcttctctatatgagcctgctgaccctatatttgattctttagacatagtcttcaaagatcatagccaattgagaggttgcataggtgaaaagacatcaaataatcatgaacaaataagtgttgatagaagtggtgcctcaaatggatctcttggctctcctgtactaactttaactagggatcagcttgggactatagcttggtcctatgccgtctttggacaaatggctaggagtttcttctctcatgtctggaaaactctcagccattatgaggagcaaaggatttcagagctgtatagggaggatatcatgtttgcttctcaggttcatcttgtaaaccagtgcttgaagcttgaattcccacatcttcagttgtcattatgtggtgagcttgaggataaagttgctctttctggaaaaactaaaaggtttaatcagaaaataacttcatcatttcagaaagaggttggacaccttcttgttagcaccggccttgaatgggtcaaagaatttgttgtggatggttacactttggatgctgtgattgttgataagaagcttgctctggagattgatggtccaactcatttctcaagaaatactggtgtaccattaggacataccatgttaaaacgccgatacatcactgctgctggttggaaagtcgcatcaatatcctatcagaagtgggaggaacttcaaggagcattcgaacaagtagagtatcttagtaaccttctcaaaaatcatctagatgaagtatatgacgaaactacatccaccgaggtaaagtgatcatccaatgctgcttgtcaagcataaggagatatcagatatgtattttttttagtaagctcccttgataaattttggaataggggatgtatatttcttagaaatattacattttaaaagaatataaataccatttcttctgttttggagtaaaatatcactttattagtccctaaattcaacaccacaaatgattttggttccttatattacacaatgcaaagttattccttgactttgataactcttaccctatgttaggtaggag

SEQ ID NO: 12 - DNA, 1233 bp, Glycine max
  source 1..1233: mol_type = genomic DNA
atgttctctttaaccagcgaaaccaataattaagagagataggcatacgatgggttctgaaggaaaacctatgcttcctgttctagatttcaccatagaagatttgaagccagggaccaattcctggttatcaacatgcacaagtgtgcgccaagcatttgaggagaatggttactttgttgctgtgtatgataaagcttctattgagctacaaaatggtgtatttggctccatgaaggagctgttcgatctcccaacagaaaccaaacgaagaaacatatttgagggcatgcccttgaaaggctacgttggacagcaccccaagattcccctccacgaaagcatgggcatagatccgggaacaacactagagggaattcaaagtttcgcagaaaaaatgtggcctcatggaaatgatcaattttgtaaatatatatttgaatatgcaaaggtagctgaagtattgaatcggatggtggtgagaatgatatttgaaagttatggtcttttggagcactatgacacattcatagggtctacaaattatctccttaggcttttggcacacaaagcactcgaacagaatgagcctcaactgggttttgtggctcacactgacaaaagcttcacaactattcttcatcaaaatcatgtcaatgcactcatggtggagacaacgaacggtaactggattgatgttgatttctcttcacctacttcttttgttgtaatggctggtgatgcactcatggcttggagcaacgacagaataaagtcccctaatcatatggttatgatgaatgggaatgaaacaaggtactctctaggcctgtttgccttctacagaggaattctgaaagtacctgaagagctaattgatgaagaacatccattgcaatataagccgtttgatcatcttgcattactaaacttcacttacagtgctaatatgaaagcctattgtggcgtgtgaaaagcttgccaaggttaaactttcagcaaactgtgtgatatgtatttcatatagttcttcctttgcaaatagctacactaaactttggttgtgatgtgatatttattatctggtcagttatgtattctagatttggcttgtattataagtgctttccaaataaattgctgaatgtttatcatcgtgtaaatgatgttgtattaaaactctgtatatcggtgcggctaagttgcagttttaagatcctttttaatttttaa

SEQ ID NO: 13 - DNA, 957 bp, Glycine max
  source 1..957: mol_type = genomic DNA
gaggattccttcgtctctgtctttgtctttctctctctgttcactttttctatcctcgaagcaagaaatggactctaacagcgtcgtttcttggccgaggatgccagaaaacatcaacggtttcggcctcaaacagaaccaatccgaagaggttcatgttttagccgttgatgacagcaccgttgatcgcaaagtcatcgaacacttgctcaaagtcttagcctgtaaagttactgctgtggatagtgggctgagagcactgcaattgcttggactattggacgagcagaaaataccctctgaaactaatggttttgtgggtttgaaggtagatctaatcatcacagactattgtatgcccggaatgaccggttatgaattactcaagagaatcaaggaatcatctactttcaaggaaacgcctgtggttattatgtcatctgaaaacgttttgccacgcatagacagatgtttggaggagggtgcagaggatttcatagtgaagccagtgaagttatctgatgtgaagcgtttgaaggattacatgacaacaaaggaggttaggggtgaattaagccaagaagagagggaagggatcaacaagaggaagctattggacacctctgatgtgtcgccatcatcaccaccgtccatttcctcatcaccacttccatcttcactcagctcagtgattgactctccaatcagacggcttaaaatgaccaacactgattgatatactctagcaatttgctgtttttcttttctcattcctgtaggcactactgctgcaatctttggatttgttgtttttcccttttagggttcaaattgcaattacctcctcgtttaattgtaacggtttttgttgcgctcccctcttcttatttttactcagttattttaattgtttatatatgaaggttaattgattaataaagttgttcacttaatact

SEQ ID NO: 14 - DNA, 1656 bp, Glycine max
  source 1..1656: mol_type = genomic DNA
atggcaggtcttccattactacacagatgtgtacaaagaggtgattgggacactgcacgcacttttgtgaacaacaatcgtaaggcaatgtatgaaacttcaaaactgggcaagacggttgttcatgttgcagttctcacaggacaagaagacatggtggaaaagttagtaaacaaagtgcccaaacggcttttgttggaacgtgacacaagagggtacacggcactggctcttgctgcagagttgagtgacaccatctccgtggccaaatgcatggttgatagaaacagagacttgttaacaatcaagactaatgaggggctgataccacttgttttggctgctgttaaaggcaacaaaaacatggccaaatatctctatcacaacactcctaagcaagtgttcaatgaggacaatggctacaccagtgctttgcttcttaccagatgcatcactagcgagatctttgacgttgcattgaatctgctcaatcgtaatccaagaatacctctcaccatgaaatttgatggtgttagccctttatttgcattggctcaacaaccttctgcattccccagcgtaaatccgcctaaattatggctacagtgggtgtataaaaggataaagaaaacacacaagaaaaaaatgactcaccatcgtgcagttgagattcttaatagcatggctaaaggaattatgggctttgatgaaacaaagctgagagaggcctcagtttacgagtccttattagaagcgtccaaaagtggaattgcagagttcataatcaagttgactcaggctaatcctgatctttattgggtttttgatgaaaatcaaagaggcattttttcttatgccattttgtatcgtagagaaaacattttcaacctcataaatggcttgaaaggacaaggaaaggtgattatttctcgcacagatatatttggtaacaatatgttgcatttgatagggacttcagtgcccacggccgagcttgatcgcaaatctggtccagctttgcaaatgcaaagagaacttcaatggtttaaggcggtgaaaagaattttgcatccaaagtttcaacaagctataaatggagatggcatgaagcctaaagaattatttactaagaaacatgaagaactgttgaaggacgcagagaaatgggcaaaggagacggcaacatccttcacaatagtggggactcttataataaccattgtgtttgcagcagccttcactcttcctggaggaaatgatcaaaatactggtattcctatgtttttgcacaaaagaatgttcacaacttatatggtggctgatgctatctcactcttttcttcctccactgctgtgatgacttttattggaattctcacatctcgttatgctgaaagagatttccttaaatccttgccactcaagttgatgtttggccttttcacactcatatgttctatcttggccatgatggttgcattttgctctgccttttctttgatgcttgaggattcgggtcattccaagatggtaaaatttgtcatttccattgcaagcttatctgtggtcatattcttacctatgcaacttcgtctcctacttgagatttttaattccacctttagatcagaggtgttgaatatcaagaaagtataa

SEQ ID NO: 15 - DNA, 943 bp, Glycine max
  source 1..943: mol_type = genomic DNA
agtgccaaagtccgtaactaattcaataacatagaagaatatggaagatggaagcgtcaaccttcctcctgggtttttcttctctcctactgatgaagaacttgttcttcactttctctattgcaaggcttctctcattccatgccatcctaacatcatccccgatctccatccgtctcagcttgctccttgggaattaaatggtaaggcgttgttaagtggaaatcagtactatttctttaccaaagtgaatgaaaacagagccagagaaaatgggtattggaaggacacaggtgtcacggaacctatattgtcaacctttgataagaaagtgggaatgaagaagtacttggtgttccacataggtgaagctccacaaggtactgaaaccagttgggtaatgcaagaatattatatatgttcctctgggtttgacactgcctcatatcgaagtgctaggagaagaagaaaacatgatcaaagttggagcaagtgggttttgtgcaaagtctatgagaagatcaggtctcaacaaggtgtaaactgctatagcgatgatgatgacagtggatcagagctttcttggctagacgaagtttatctgtcgttggatgatgatctggaagaaattggcatgcctatttagattgaatgtatactacccaatgctaatatatatatatatatatatatatatatatatggacttgtatattaattactatgtctcaattaaatacgtgcattgtttgaaacgaacgtgttcaatgtgtctcttttacatgcagattgatggcaaaaattaatgccagcgtgagtgggactaatcttgaatatcttgaaggtctgaatcttgtagagctagaaggagatacttgctcgatcctcttaagtaatacttttatgttgagtattatgataaaaaaaataattgagataaaaa

SEQ ID NO: 16 - DNA, 8122 bp, Glycine max
  source 1..8122: mol_type = genomic DNA
gacttttgactctctctctctctctctctacagtagtagtagtagtattcattcatattcatacttgatatcatgtcacacacacactatctctctctctctctctctaatggcagagctagctcagacccttaacacagaccacagcctctgagagaaaccactctcactctcactctccttaacctttcaccttccatatctggcgaaaatgaaagctctgtacagaagctctctcttgctcctcttcttcctctccgtctgttctgcatcttcttcttcggttcccactctgcaactcctaagcttcaaaaactctctcccgaacccaaccctgctcccgaactggctcccgaaccaaagcccatgctcattcaccggcatcacctgcaacgacacccagcacctcacttccatagacctctccggcgtccctctcaccacaaacctcaccgtcatcgccaccttcctcctcaccctcgacaacctccagtcactctccttaaaatccaccaacctctccggccccgccgccatgcctcctcctctctcccactccaagtgcgcctccacattaacctccctagatctatcacaaaacgccctctccggttccctcaacgacatgtcgtttctctcctcctgctccaacctccaatccctcaatctctccagcaaccttcttgaattcgactcctctcactggaagctccacctcctcgtcgccgacttttcctacaacaagatttccggccccggtatcctcccctggcttctcaaccctgaaatcgaacacctcgctctgaaaggcaacaaagtcaccggcgaaaccgacttctccggttcaaattctctccagtttttagacctttcttccaacaacttttctgttacgcttcctactttcggcgagtgttcttcgcttgagtatttggacctctccgccaacaagtacttcggcgacattgctcgcactctctcgccttgcaagagcctcgtttacttaaacttctccagcaaccagttctccggtccggttccttcccttccctccggttcgctacagttcgtgtaccttgcttcaaaccacttccacggccagattcctctccccctcgccaacctctgctccactctcctccagctcgatctctcctccaacaacctctcaggcgctctccccgaagcgttcggcgcttgcacttctcttcagtccttcgacatctccagcaacctcttcgccggtgcgctgcctatggacgttctcacgcaaatgaaaagcctcaaagagctcgcggtggcgttcaacgcgttcctcggtccccttccggagtctctgacgaagctctccactttagagtcgctggatcttagctccaacaacttcagcggctcaatcccgacaacgctttgcggtggtgacgctgggaataataatattctgaaggaactttacctgcagaacaaccggttcacgggttttattccacccacgctcagcaactgttcaaacctcgttgctttggacttgagtttcaacttcctcacgggaactattcctccaagcctagggtctctttccaagcttaaagacttgatcatctggctcaaccagctccatggagaaataccgcaggagctcatgtatctgaaaagcctcgagaatttgatcctggatttcaacgacttgactgggaacattccctctgggcttgttaactgcaccaagctgaactggatctccctctccaacaacaggctcagcggcgagattccgcggtggattgggaagctttctaatctcgccatacttaagctcagtaataactctttctccggccggattccgccggagctcggcgattgtactagtttaatatggttggatctgaatactaatatgctcaccgggcccattccgccggagctgttcaagcagtcggggaagatcgcggtgaatttcatcagtgggaagacgtatgtgtatataaagaacgatgggagcaaagagtgtcatggtgcggggaacttgctggagtttgccgggatcagtcagcagcagttgaacaggatttcgacgaggaacccgtgcaatttcactagggtttatggaggtaagttgcagccaacgtttaaccataatggttctatgatatttttggatatctcgcacaacatgttgtcagggagtattcccaaggagattggggccatgtactatttgtacattctcaatttgggtcataataatgtgtctgggagcattcctcaagagcttgggaagatgaagaatctcaacatccttgatctctcaagtaatagactggaggggcaaattcctcagagtctcacggggctttccttgctcactgagattgacctgtcgaataacttgcttacggggacgattcctgagtcgggtcaatttgatactttccctgcggcgagatttcagaacaactctggtctctgtggagttcctctcggcccatgtggttcggacccggcgaataacgggaatgcgcaacatatgaagtctcacaggaggcaggcttctctggtggggagtgtggccatgggtttgttgttttccctcttctgcgtctttggtttgatcattattgccattgagaccaggaagaggaggaagaagaaggaggctgctcttgaagcctatgctgatggtaatttgcattcgggtcctgccaacgtgagctggaagcacaccagtacccgggaagcgcttagcataaaccttgcaacgtttaagaggccgctcaggaggcttacttttgcggaccttcttgacgctaccaatggctttcacaatgatagtctcattggctctggtgggtttggggatgtttacaaggctcagttgaaggatggaagtgttgtggctatcaagaagctgattcatgtcagcggccaaggggacagggaattcactgctgaaatggagaccattgggaagatcaagcacaggaaccttgttcctctgttgggatactgcaaggttggggaagagaggctcttggtttatgagtacatgaaatatggaagcttagaggatgttcttcatgatccgaagaaagctgggatcaagttgaattggtcgattaggaggaaaattgctattggagctgctaggggattgtcttttcttcaccacaattgtagcccccacatcattcatagagacatgaagtcaagcaatgtgttacttgatgaaaacctggaagctagagtctctgattttggaatggctaggcatatgagtgctatggatacacatttgagtgtgagcacactggcaggcacaccagggtatgttcctccggagtactatgagagcttcagatgctctaccaaaggtgatgtctacagttatggtgtggttttgttggagctgctaactgggaaaaggccaacagattcggctgattttggtgataataatcttgttggatgggttaaacaacatgccaagctgaaaataagtgatatttttgacccagagctcatgaaggaagaccccaatttggagatggagcttttgcagcacctgaagattgcagtttcctgtttggatgatcggcattggagacgtccaacgatgattcaagtgttgacaatgttcaaggagattcaggcaggatctgggattgattctcagtcaaccatagccaatgaagatgacagtttcaatgcagttgaaatggtggagatgagcattaaagaaacccctgaattgagcaagcattaggcaacaaattccggaagtgtcatgtggattctttgggaaacagacaagaagagagaaaaggaggagatgacggattcagctcccctcaaagttttttccttcctctttgccgcttcaaattatttcagacacaagggggaaacggttaaaggggggaatgaatgcctttgatgtatgtagtcttgttattttatacaaaaaaaaaaagttgtttaacttgtatataaacagcttcagttgttaccatctgtgttttcctagaattttcaaaagtacttttctcattcagatataatcctgaaaaggatctctgttcaagactctctctctctctatctatggtagtagtagtagtagtattcattcattctctctaatggcagagctcacaccctaaacacaaaccagagcctctgagagaaacaaccacaaactctcccttaatcttgtcggtttccatatctgctgaaaatgaaagctctgtacagaagctctctcttgctcttgctcttgctcttcatctccgtgtgttttgcatcttcttcttctccggtcactcagcaactcctaagcttcaaaaactctctcccgaacccatccctgctccccaactggctccctaaccaaagcccgtgcacattcagcggcatcagctgcaacgacacagagctcacatccatagacctcagctccgtccctctcagcactaacctaaccgtcatcgccagcttcctccttagcctggaccacctccagtcactctccttaaaatccaccaacctctccggtcccgccgccatgcctcctctctcccactcccagtgctcctcctcattaacctccctagatctctcccaaaactccctctctgcctccctcaacgacatgtcgtttctcgcctcctgctccaacctccaatccctcaacctctcctccaacctcctccaattcggccccccgccccactggaagctccaccacctccgcttcgccgacttctcttacaacaagatttccggccccggcgtcgtttcttggcttctcaaccccgtcatcgaactcctctctctcaaaggcaacaaagtcaccggcgaaaccgacttctccggctcaatttctctccagtatttagacctttcttccaacaacttttctgttacgcttcctactttcggcgagtgttcttcgcttgagtatttggacctctccgctaacaagtacttgggcgacattgcacgcactctctccccttgcaagagcctcgtttaccttaacgtctccagcaaccagttctccggtccggttccttcccttccctccggttcgctacagtttgtgtaccttgctgcaaaccacttccacggccagattcctctctccctcgccgacctctgctccactctcctccagctcgatctctcctccaacaatctcaccggcgctcttcccggcgcgttcggcgcgtgcacttctcttcaatcccttgacatctccagcaacctcttcgccggcgcgttgccgatgtcggttctcacgcaaatgaccagcctgaaagagctcgcggttgcgttcaacggcttcctcggtgctctcccggagtctctgtcgaagctctccgctctggagttgctggatctgagctccaacaactttagcggctcaatccccgcctcgctgtgcggcggcggtgacgctgggattaataataatctgaaggaactttatctgcagaataaccggttcacgggttttattccgcccacgctcagcaactgttcaaacctcgttgctttggatttgagtttcaacttcctcacggggactattcctccgagcctagggtctctttcgaatctcaaagacttcatcatctggctcaaccagctccacggggagataccgcaggagctcatgtatcttaaaagcctcgagaatttgatcctggatttcaacgacttgactgggaacattccctctggcctcgttaactgcaccaagttgaactggatctccctctccaacaacaggctcagcggcgagattccgccgtggattgggaagctttctaatctcgccatactcaagctcagcaataactctttctccggccggattccgccggagctcggcgactgtactagtttaatatggttggatctgaatacaaatatgctcaccgggcccattccgccggagctgttcaagcagtcggggaagatcgccgtgaatttcatcagtgggaagacgtatgtgtatataaagaacgatgggagcaaggagtgccatggtgcggggaacttgcttgagtttgcggggatcagtcagcagcagctgaacaggatttcgacgaggaacccctgcaatttcactagggtttatggaggtaagttgcagccaacgtttaaccataatggttctatgatatttttggatatctcgcacaacatgttgtcagggagtattcccaaggagattggggccatgtactatttgtacattctcaatttgggtcacaataatgtgtctgggagcattcctcaagagcttgggaagatgaagaatctcaacattcttgatctgtcgaataatagactggagggccaaattccgcagagcctcacggggctttctttgctcactgagattgacttgtccaacaacttgcttaccgggacgattcctgagtcgggtcaatttgatactttccctgcggcgaagtttcagaacaactctggtctatgtggagttcctctgggtccgtgtggttcggagccggcaaacaatggaaatgcgcaacatatgaagtctcacaggaggcaggcttctctggcggggagtgtggccatggggttgttgttttccctcttttgcgtctttggtttgatcattattgccattgagaccaggaagaggaggaagaagaaggaggctgctcttgaagcctatggtgatggtaattcccattcgggtccggccaatgtgagctggaagcacaccagtactcgggaagctcttagcataaaccttgcaacatttgagaagccgctccggaagcttacttttgcggaccttcttgacgctaccaatggttttcacaacgacagtctcattggctctggcgggtttggggatgtgtacaaggctcagttgaaggatggaagtgttgtggctatcaagaagctgattcatgttagcggacaaggggacagggaattcaccgctgaaatggagactattgggaaaatcaagcacaggaaccttgttcccctgttgggatactgcaaggtaggggaagagaggctcttggtttatgagtacatgaaatatggaagtttagaggatgttctacatgatcagaagaaagctgggatcaagctgaactgggccattaggcggaaaattgctattggagctgctaggggattggcttttcttcaccacaattgtatcccccacatcattcatagagatatgaagtcgagcaatgtgttacttgatgaaaacctggaagccagagtctctgattttggaatggctaggcttatgagtgctatggatacacatttgagtgtgagcacattggctggcacaccggggtatgttcctcccgagtactatcagagcttcagatgctccacaaaaggtgatgtctacagttatggtgtggttttgttggagctgctaactgggaaaaggccaacggactcggctgattttggagataataatcttgttggatgggttaaacaacatgccaagctgaaaatcagtgatatttttgacccggagctcatgaaggaagaccccaatctggagatggagcttttgcagcacttgaagattgcggtttcctgtttggatgatcgaccgtggaggcgtccgacgatgattcaagtgatggcaatgttcaaggagattcaggcgggatccgggattgattctcagtcaaccatagccaatgacgaggaaggtttcaatgcagttgaaatggtggagatgagcattaaagaagcccctgaattgagcaagcattaggcattaaagtgtcgtgtggattctttgggaaacagacaagaagagaggaggagatgacggattcagctcccctcagttttttccttctttgccgcttcaaattatttcagacataagggaaaacggttaaaaggggaatgaatgcttttgatgtatgtattcttgttattttatacataaaaaaaagttgtttaacttgtatataaacagcttcagttgttaccatctgtgttttcctagaattttcaaagtacttttctcattcaaatataatcctgaaaaggttctctgttcaaaaaaaaaaaaaaaaaaaaaaaaaaa

SEQ ID NO: 17 - DNA, 3835 bp, Glycine max
  source 1..3835: mol_type = genomic DNA
cactccaaggtgttatctttatctcacaacttgtgtgttctgtcttgttttgatttcagatttctagtgcttttgtgtttttgttggaagatggctaaacttgcagttccacctttgctttctctgaggcacttcctggtgctatcaattgctttgaatgtgagtttgattctgaggactgtgtattatgagagtgaaaaaggaagcaaagggttttgtttgaacaaaaaaatgatggcagatgcagattctgatagagaggggcatcttatccacaaatcacgtttggccatgtcctctacttcgtctctcgttaattctactcgtgcagatcatccaggaggaagggaaacagtgatcaacttggaccatggagacccaacaatatatgaaagattttggagacaagtgggtgacaagaccacaatcataattccaggatggcaatccttgagttatttttctgatggctcaaacatttgctggtttttagagaccgaatttgcaagggaagtagtgaggttgcacaaagtggtgggtaacgctgtgacagaaggtcgccatattgttgttgggacaggttcctctcagctaattctggctgccctttatgctctgtcctcgcctgatgctgctgaaccaatcagcgtggtatctgccgcaccctattattcgtcttacccatcaatggcagattaccagaaatcaggactttataaatgggctggtgatgcagagaattttgacaaagagggtccttatattgagcttgttacttctcccaataaccctgatggacatagaaggaaagcaatggtcaaccgaagccagggacaattcattcatgaccttgcctattattggcctcaatacactccaatatcatccccttcagatcatgatctcactcttttcactgtctcaaaaaccactggtcatgcaggaatgcgcatagggtgggctattgtcaaagataaagaggtagcaaagaaaatgactaaattcatagagataagtacaatcggtgtctcgaaggattctcaactcagagctgccaaggttttaaaggcagtgtctgacagctgcgaacaagaaaactcccaagatggggaatcatttttcacgcacagctataacataatggcccaaagatggaagcaactgagagcagtagttgaggccggtgatttgttcactttgccacaattttctcctgcattctgcaccttctttggtcaggagacggagcctcaaccagctttcatatggttgaagtgtgaaggagatatagaggactgcgaaagcttgcttcgagaacacaaaattgtagcaagaagtgggagacactttggggctagcccaaaatacgtaagaattagcatgctggatactgatgaaacttttatacagttaatagatagactatcagccatacaacaaggaaaatgaaaaatttatccctgatgtatatgtataacaaataaaaggtcatagttcatatataatattcggccgtgcaatattctctgatccattcatatataagaatattgttttctccttaacataataccccagtacaaatcccttgtgagtagcaacctttattcctctactttctttctgtccataagtagagttgctccatatgagactctctcttgcttcccgctttctcctccattacaacaaaaccgaacttcaaactatccctcaccctcagtttgctgaagcaaattagaccttcacaataccctatttccaaaatccaatattcttcataacatcaatctcatcccgtcagctgattaattctttataggaggggagagttggtgattaaaagagttagttgagtttcttttatggttgtttcactttgatataacttcagcaccatctaaacctttgtagctagctaggcttcaaagtcttgaaatcatctaccttaaactccaaactttccaccttttttatttttgtttttgtttctggggtggtgttcatggactacttgaaggaactagaaggaaaaagtgtccatgattgttatcaccatcaccagcagcagcagataaagatgagtaaaatggcaagccctatatttgttccgggaccagttatagtaggagctggtccctcagggctagctgcagcagcttgccttaaacagaaaggcatcattccaagcctcatccttgaaagagctcaatgcttagcttcaatgtggcagttcaagacttatgatcgcctcaggcttcatctcccaaagcaattctgccagctccctctcatgccattccccaaaaacttgccttcttatcccacaaaacagcaattcttggcctaccttaaggcctatgctgaccactttgacataaaacctgtcttcagccagactgtggtcagtgctgaatttgatcatgtgtgtcagctttggagggtcaagactcgaggagtaatcaagaaagaggacacagcagaatatgtttgccaatggcttatagttgctactggagagtgtgctgaggaagtagtgcctcaaattgaagggatgggagagtttgaagggcagattgtacacactagcaagtataagagtggaagcatgttttgtgggaagaatgttttggtggttggttgtgggaattcaggcatggaggtttgtttggatctctgcaaccataatgctcgtccttccctagtggttagagacacggtgcatatcttgcctcaacagatgctagggaaatcaacttttggtttatccatgttcttgctcaagtggttccccatacgttttgtggatcaatttttgcttctaatgtcacatctcatgcttggagacacagctcaatttggacttcgtcgtccaaaacttggtcctctagagctcaagaacttgtacggaaagacaccagttttggatgttgggacactcactcagatcaaaaatggaaaaattaaggtttgccggggaattaaacgactagcacgcaatgcagtggagtttgttgatggaaaagtagagaactttgatgctatggttctagcaactggttacaaaagcaatgtaccctcttggttaaagggtagtgacatgtttagtgaaaaagatggatttccccggaagccattcccaaatggatggaaaggtgaaaatggactctatgcggtgggtttcaccaaacgtggccttcttggtgcatctattgatgcaaagaggattgctgaagatattgaacatagctggaaagctgaggccaagcatgttttggagttccctcgtccacttgcgtgatgtgggaaagatcgatgcaaatgttttaggattaattagaaaaaaacaaaaaaaaaaagtatggccaagcctcttggtggtgccttttatttgacacaatgctttgtaccaaactatattttctctcccattttagtacagcagcctcaaaaggggaaaaaggagttttagaaagaaaaggaaaattaagaaaaagaactaaaatgagaggccatatatgtgtgcatagaatgtgtgattcctataaatccattttcaccaaactaggagattgggttggcgactaatttgatgcgtatttttagtttggttgggttgaaattaattagctgaatattgtcatattaattacacattatttgtatatctttttgtacattgccttatctacctagttagctagttgattttattttttatttttttttagaaaaggggttgttgttatagttttttagcctatagaaattatgtaatgtatccatcgatttggccttgttctaattatttatgatagaattgcaagctttgtga

SEQ ID NO: 18 - DNA, 9304 bp, Glycine max
  source 1..9304: mol_type = genomic DNA
aattgatatgagacttacggattcctatgaggcaaacgaattgactattttttcttctctgaaaaaaagagataaatacagtcagaactgtgttgggaacagaaagcaactttgttggcagataaccctgaaaatctctgaccactggcaacctcacgatcaccaaatcaacaattgaaacccaaaactacctttgaactcgcaagcaacctctccagttgatatgaaccatcgaaatggtaacatgcataaaaaacaaaacaaccacgtgaaccaaatccacatgttcagaacaaaacacacaaaacaaccccgctcccacctcacttgaaaaacctggaccgttcgcaaagacgactattggaatcagcatcagacgtttctacctcactccggcatgctcaaatgatagttttctctctccctcgatttcctacacactcgaatcttgcttctcttttgtactttgttttgtagaatttgcctttttttctcttttgtagttatagaagtttggcgatttaattggcgttaaacgaccccattttggttgcgtgttctcgcttctcaagttatgtgaaaaaggaaaaatcgcctgaggtggttcaaacactatggaatcctgcaattgtattgacccacaagtgccagctgatgacttattgatgaaataccaatatatctctgatttcttcattgcccttgcttatttctcaatccctttggagcttatctactttgttaagaaatcagcagtatttccatatagatgggtccttgttcagtttggtgctttcattgttttatgtggagcaacacatctaattaatttatggacatttagaattcattcaagaactgtggctgttgtaatgaccactgctaaggttttaactgctgtggtatcgtgtgcaactgccctcatgctggtacatattattcctgatttattaagtgttaaaactagggaattatttttgaagaacaaggctgcagagcttgatagagaaatgggcctgattcgcacacaagaagaaactggtcgacatgttaggatgttgactcatgagattagaagcactcttgatagacacacaatattgaaaacgactcttgttgaactgggtagaactttggcactagaggaatgtgcgttatggatgccaacacgaactggtttggagcttcaactctcttacacattgcgacagcagaacccagttggatacacagtacccattcatcttcctgttatcaatcaagtatttagtagcaacagggcagtaaaaatttcacctaattgtccagttgctagattacgaccttatgctggaaaatacatgcctggggcagtagtagctgttcgagttcctctcctacatctttctaatttccaaatatatgattggcctgaggtttcgacaagaagttatgctttgatggttttgatgcttccatcagatagtgctaggcaatggcatgtgcatgagttagaactggttgaggtagtcgctgaccaggtagctgttgctctttcacatgctgcaatcttagaagagtcaatgagggcaagggatcagctcatggagcagaatgttgcattggatctagcaagaagagaagcagaaactgcaattcgtgctcgtaatgactttttggctgttatgaaccacgagatgagaactcccatgcatgcagttattgcactctcttcattactacaggaaacagatttgacggctgagcaacgtctgatggtggagacaatattgaaaagcagcaatttgttggctaccctcatcaatgacgttttggatctttcacggcttgaagatggcagtcttcaacttgaagcaacaacatttaacctccattctttgttcagagaggtccttaacttgattaagcctgttgcatctgttaaaaagttatcactcacttcacatatagcctcagatttgccaatgtatgccattggtgatgaaaaacgtctcatgcaaactattctgaatgttgttggtaatgctgtgaagttctcaaaagagggctgcatttccatctctgctttcgttgcaaagcctgaatcctttagagatgctagaattcctgactttcttccagtgctaagtgataaccacttttatttgcgagtacaggtaaaagattcaggatcaggaattaatccacaagatatcccaaagatattcactaagtttgcacaaaaccaatcgttaacaacaagaaatcctgctggaagtggacttggcctggcaatttgtaggaggtttgtaaatcttatggaaggacatatttgggttgaaagcgaaggtattggtaaaggatgtacagtcacttttattgtaaaacttggaatcccagaccgatcaaatgaatttaagctaccttttgtacctaaagtaccgggaaatcatggatctacaaactttgctgggctcaaagttcttgtcacggatgacaatggggttagcaggacagtaacaaagggacttcttatgcatttaggatgtgatgtaaccacagcaagctcaagtgaagaatgtctgcgtgttgtttctctggaacatgaagtggtcttcatggatgtttgtgcagggttagatggttatgaacttgcaatccgcatacatgaaaagtttactaaacatcaagatagaccactaatagttgctcttactggaaacaccaaaaaggtaacaaaagaaaactgtatgagggttggtatggatggccttatattgaaacctgtttctgttgacaaaatgaggggtgttttatcagaactattggagcgccgagttctgttcgaaactgtttaacctgttagattgttgtatatagctacatcaaatttggagaaacacaaacttatgaaaacatgaggcaattgacattaattttcatcctttttgcttttatgagatcccggcgaaaggaacaacgattgagtctacgttgttaaatcattttttatgaacagaagttcgtatatgttgtatattctgcatttttatagcagtctttcaatgtttttcaatataagtgatattatgaaatcttatgttttaattaatacagtggttgatgcaaatggaaaagactaaagacattttattattacatgtaaaggtgtgttagcagagcaagggagtcccggttcagttcatgtaaagctgtgtgggtcccactccctctctctctctctctgctttcaagacctatcagtcaagtgctcccttcaaacttaagattgaaattttcttctttgtcgttgggaagccgtgatttcttcttatcttctctgtttctacccccaaattgttagtggtgtgagcttttcctgttttatccaatcagttattatgagtctatagttagtggtggcattatttatttatttatttaatcgtttgaatttatctaaattgggaaagcctatttataactcgaagaactttatgtgaaattaaacttagtttagatctagtatattgcaacgtgtatggcaattgtgatgtcaatttatttgagctattaacttagaaaaggaaaacagtagtacttagttgttgccatggaaagagggttgcttcttttgttgttcttgttgttggttatggtgctttctgtttgtggcaacgatgtggaatattcacagtgcaattgtgacgaggaagggctttggagcatccacaacgtgctggtttgtcagaaattgtttctttccctcactgttgctaagttcctcactgctcttgtttcttgtgccactgcaatttcctttcccacactcattcctcttctgctgaaaatcaaagtgagggagctatttttgaggcagaatgtgttggaattgggccaggaggttgggatgatgaagaaacagaaggaagctagttggcatgttaggatgctcacttgtgagattaggaaatctctggataagcataccatcttgtatatcacacttgttgagctttccaaggcattggatttgcacaattgtgcagtatggatgcctgatgaggataggagagaaatgcatttgactcatgagttgaaaccaaactcaacaaggatttttcacaattctattcctataagtgatcctgatgtgttggacataaagaagagccaaggggtgtggattttgaggcctgattctgcactaggagctgcaagtagtggtggtggtggatctggagattctggtgctgtggctgccattcgcatgccaatacttcatgtttcgaatttcaaaggaggcacaccagagtttgttgaaacatcttatggtgtactggttttggttcttccaaattcagactccagggcttggacctctcatgaaatggagatagtgaaagttgttgctgaccaggtggctgtggcattgtctcatgcgtctgttcttgaagagtctcagcttatgagccagaaacttgcagagcaaaaccgggcgttgcaacaggctcaaaagaatgcaatgatggccaggaaggcaaggagctcgtttgagaaagttatgagtcatggaatgcggagacctatgcattcaatcctgggcttgctttcaatgtttcaagaggataatataagacctgaacagaagattgttattgattcaattttgaaagttagcaatgcactatcacgattgattaatgatgtgatggagattgcggcaaatgacaatggaagcttccagttagagatgaaacctttccatctacattcaatgatgagggaagcttcttgcactgccaagtgtttgtgtatatataaaggctttggtcttgaagttgatgttgacaagtctttgcctgatctggtcattggtgatgaggcaaggactttccaagtaattttacatatgattggctatttattgaacatatatgataaagggaatctcatttttcaagtttatcttaaaagtgatagtggagatagagatgacagaagttttggattatggagatcgagcatgcaaaatgagtatgtacatattaaatttaattttcagataaatggtataagttcccagtcagatgaatcagtttcaacaagaaattatactggtagaaggcactacaacaatgaacctaaggaaggcctgagcttcagcatgtgcaaaacgctggtgcagatgatgcaaggtaatatttggatatcaactaactctctgggtttggcacaaggaatgacgcttcttctcaagtttcagataggatcatcccatggaagattcactcttgctcctacagatttttcaaactctcagtttagagggcttaaggttgtgttggctgatgatgatgatgtaaacaggactgtgaccaagaagctgcttgagaaactggggtgtcaagtaactgctgtttcatcaggattcgaatgtcttggtgctataagtggctctggtaactcattcaaaattattctgttggatcttcacatgcctgaaatggatggttttgaagttgcaagaaggattcggaaattccaaagccataattggcccttgatcatagcttttactgcaagtgcagaagaacacatcaaggagagatgtctacaggtgggaatgaatggattgattcgaaaacctatcctccttcgtgagatagcagatgaacttggaacagtcttgcaacgtgcaggtgaaaaattgtgacaacaattttaaatatctcaaattttttaggttcctcaaaattcattactttgacattttgaaagagaacctgaaattcttaacaaactaaatatactagatactaggttctaggttcattttgctaaccattttttctgtgtgatctttttaccgtacaatgaattatggatttctacaaggtgtatagaacggcttcccaccatcaaatagaaaagtatgaaatgcaaaattctgaaattgcgtatattctaaatccaactcacccctttctcttctcccacgaaacaacggcgctttctctctccatggttttgtgctttggtttcgcattgcaaaatacctcaaaagcttaagactttaagattccctgcatacaattcaacacttcattctttttttttttcttcttcacctcgcttgggttggctgtggtgtagggactgcgttttcctctctctgttctcttgtttgctacgctgagcactggccttttccgcaaaagggtcgcacccggtttccctgtggaacagtgaagatggtttcttttttatttttctaattgatatgttgttgggatttggtgactccttctagtgggtttctttctttgatcttcccaatggtggtggaggtgggtgcatttgttgttgttgtgtataataaagtaatggactttgctggtggagccttttgatttgatgctcaacaaactactacaggagatttggttaatgtgttgttgctattgaggtatcttcaacaaacaaggctttggtctttccagggtaaggttaaagatctgatgaaggtggaatatctgattgcattaatggcttgttgtaattcctcagggtggcatgaaagaaagaaggttaaaactgaaattccaagattgaaatgttgtagatcgtttaatttgggacaagaattaaatacttggcaaggcaattgaaagggaggaggttgggggggtgttcactggggtatgactatggcccattaaattatatccttagatggattgaggaagtagcaacattaatttggatcaatgttaaaagcagtggcatctgggcttgtgattacttctatcctcatatgtgtatctgcaaatgataatggatttcctagatgtaactgtgatgatgaggccagcttgtggactattgaaagcattctggagtgtcagagagttggtgatttcttgattgctgtggcctacttctctatccctattgagctgctttattttatcagttgctcaaacgtcccattcaaatgggtcctgattcagttcattgccttcattgtactatgtggattgacacatttgctgaatgggtggacctatggccctcacactttccaacttatggtggcacttactgtctccaaaatactcactgcattggtgtcatgtgccactgccataaccctcatcactctgatcccattgcttctcaaagtgaaggtcagagagttcatgctgaagaaaaagacgtgggatcttggacgtgaggttgatcatattatgagacaaaaggaggctgcaatgcatgtaagaatgcttactcaggagatacgtaagtcactcgatagacatacgattctgtataccactttggtggagctgtctaaaacacttggcttgcaaaattgtgctgtgtggatgcctaatgttgataagactgagatgaacctcactcatgaattgaatgggaggaattttaatttgactatacgcattactgatccagatgttgtgagaattaagggaagtgacggtgtaaatatactcagctccgactcagcacttgctgttgggagtagaggagtttctggagaggcaggaccggttgcagcaattcgaatgccaatgctacgggtttgtaatttcaaaggaggaacacctgagctaagacaggcttgttatgctatattggtcttaattcttccttctggagataatcaggagcctagatcgtggagcaatcaggagctggagataattaaggtggttgctgatcaggtcgcagtggctctatctcatgctgcaattcttgaagagtcccaacttatgagagagaagttggaggagcaaaatcgagctttgcaacaggcaaaaaggaatgctttgatggcaagccaggcaagaaatgcatttcagaaagtcatgagcgatggaatgaggaggccaatgcactcaattttgggactgctttcaatgatacaagacgataaattgaagagtgaacagaaacttattgtggacgcgatgctaaggaccagtaatgtcctatcaaacttgataaacgatgccatggataactcaactaaggatgaaggaagatttcctttggagataaggtcttttgggttacattccatgctgaaggaagcagcttgtctttccaagtgcatgtgtgtttataagggctttggttttatggttgaggttgagaagtgtttgcctgacaatgttatgggtgatgagaggagggtttttcaggtaattttgcatatggttgggaacctactggaacacaaccatgggggagggatccttgtatatcgagtttttgcagaaactggaagtcagggaaggagtgacaaagggtggacaacctggagaccaagctcttctagtggtgatgtaaatattagatttgagatagggatcaacagtagtgattctgaagttgggagctcaatttcttcaggatttgggggtagaaaatatagcagtgatagggttgggggcagattgagcttcagcatttgcaagagggtagtccagttgatgcaggggaacatatggttagtaccttgtaatcatggttttcctcaaagcatgactcttcttcttcggtttcaattacgaccgtccatttccatagccatctctgaccctggagaaggttcagagcgcactgattccaattcaatgctgagaaatctgcaagttcttttagtcgaaaatgatgatgtaaatagagcagtgacccaaagattgcttcagaaactaggttgtgttgtaactcctgttgcttcaggatttgaatgccttactgtcattggacctgcaggatgttctattcaagtcattcttttagatcttcacatgcctgatttagatggctttgaggttgccacgaggattcggaagtttagaagtggtaatcaacctatgattgttgccttgactgcaagtgcagaagaagatttgtgggaaagatgcatgcaagttggtattaatggagttatccgcaagccagttttgttgcatggaattgctagtgaactcagaagaatcctcatgcagggaaataatgtcctttgagaatggaatggagggtgcaagttgtaacacttaagcatctcaagtatttaagttattattttaagtcacattcttggtagaattagaacacagcactctttggctcccacttgctagcaatttttagcttagagatgttattatacaaacgattttcataaggaagaaaaagatggtatacagattagttccttttcagtagtgtcttgacggtgttattgccatgtatgtagcttcaaaatctaatacatgcgtcttcccacata

SEQ ID NO: 19 - DNA, 2131 bp, Glycine max
  source 1..2131: mol_type = genomic DNA
ttgaactaaagagattcctgtctctgaaggtcaaggtgaatttgtgcactacattataaataagcacccctctcggtgttgttgttcattgctagacatttcctctgaaaattattcatagcatatttctaaccacactcaaaaggcttcccttcctgacttggatttcattacattgctaaactatattatttttattggtgaattgctacactatattctaaggacaattaaacatagactatatggggttgatggatgtggaccaaactcaattgttgtctaagatggtcatcggagatggacatggtgaagcatcaccatactttgatggatggaaggcttatgatgaaaacccctttcatcccaaagagaatcctaacggggttatccaaatgggtcttgctgagaatcagcttacttctgatttggttgaagattggatactgaataacccagaggcctccatttgcacaccagaaggaataaatgatttcagggccatagctaactttcaggattatcatggtctgcccgagttcagaaatgctgtggctaaattcatgggtagaacaagaggaaacagagtcacgtttgatcctgaccgtattgtcatgagcggtggagcaactggagcacacgaagtcactaccttttgtttggcagaccctggtgacgcatttttggtgcccattccttattatccaggttttgaccgggatttgaggtggagaacaggaattaaacttgttccagttatgtgcgatagctcaaacaatttcaagttgacaaagcaagcattggaagatgcgtatgagaaggccaaagaggataatataagagtaaagggcttgctcatcaccaatccatcaaacccattaggcacagtcatggacagaaacacactaagaaccgtgatgagcttcatcaacgagaagcgtatccaccttgtatctgatgaaatatactctgcaacagtttttagccaccccagtttcataagcattgctgagatattagaggaagacacagacatcgaatgtgaccgcaacctcgttcacattgtttatagtctttcaaaggatatggggttccctggcttcagagttggcatcatatactcttacaatgatgctgtggtccattgtgcacgcaaaatgtcaagctttggattggtgtcaacacagactcagtatcttttagcatcaatgctaaatgatgatgagtttgtggaaagttttctggtagagagtgcaaaaaggctggcacaaaggcatagagttttcactggggggttggccaaagttggcataaagtgcttgcaaagcaatgctggtctctttgtgtggatggatttaaggcaacttctcaagaagccaacgcttgactctgaaatggagctttggagagtgatcattgatgaggttaagatcaatgtttcacctggctcctctttccattgcactgagccagggtggtttagggtgtgctatgccaacatggatgatatggctgtgcaaattgcattgcaaagaattcgtaactttgtgcttcaaaacaaggagatcatggtgcctaacaagaaacattgttggcacagtaacttgaggttgagcctcaaaaccagaaggtttgatgatatcatgatgtcacctcactcccccatacctcagtcacctctggttaaagccacaatttgagttggcatatttctctgaaccctctagaagaagtaactgatatatgatgattatttggctctttgacttgttgttttggcaaggtacataaagtgcttgagtttgttatttttaacagcagtaacaggcaatgcctgatattgtttttgttaccaacatcgaagtctctttgccttgcgtcatagaacttgtgctactggaaacatgccccttcaatttttaggggcattttttttttcttttttcacttaccaaataatgggttcaaggtgaaaaaagtttataatgtttgtaaggttattggtttattagaagagtccagaagaagtctgtattcttatactgcaattgtaaggtcgatgatcaataaattgttaatagagggtcttcaatacccaccctcttttctttttgagacaaaaggaaatttg

SEQ ID NO: 20 - DNA, 3978 bp, Glycine max
  source 1..3978: mol_type = genomic DNA
atgtcaggtggtggatgcagcagcatagtgtggttcagaagagatctgaggatagaagacaatccagcactgacagcaggagtgagagctggtgctgttgttgcagttttcatatgggcacctgaagaagaaggacaatactaccctggcagggtctctagatggtggctcaagaacagtttggctcaccttgattcctctctcaggaatcttggcactcctctcatcactaagcgatccactgatactctttcttctctatttgaggttgttaagtccactggagccactcaactcttttttaatcatttatatgatcctctgtcacttgtcagggatcaccgagcaaaggaggttctgactgctcagggcataacagtacgttccttcaacgcggacttattatatgaaccatgggatgtgaatgatgcacatggccgaccattcacaacttttgctgctttttgggaaagatgtcttagcatgccttatgaccctgagtcacctcttctcccacctaaaaggataattccaggtgatgcatcgaggtgccctagtgatacattgctgtttgaagatgaattagagaaggcaagcaatgcacttcttgctcgagcgtggtcacccgggtggagcaatgctaacaaggcattgacaacattcataaatggtccactgattgagtactcaaaaaaccgcaggaaagcagacagtgccactacctcacttctctcaccccatttgcactttggtgagttgagtgtcaagaaggtcttccatctagtccgcatcaaacaagttttgtgggccaacgaaggaaacaaagctggtgaagagagcgtgaacttgttcctcaaatctataggtcttagagaatattcaaggtacattagtttcaaccatccctacagtcatgaaaggcctcttctaggccaccttaagttctttccttgggttgtaaatgaaggatattttaaggcatggagacaaggaagaactgggtaccctttggtggatgctgggatgagggagttgtgggccactggttggctgcatgatcgaatacgtgttgtagtttctagtttctttgtgaaggttctgcagcttccttggagatggggaatgaagtatttctgggatacccttttggatgccgatcttgagagtgatgctcttggttggcagtacatatctggtagtctccctgatggtcgtgaaattgaccgaatagataatccacagtttgagggctacaaattcgacccaaatggagaatacgtgcgacgctggcttccagaactttcaagattacccactgagtggatacatcatccttggaatgcaccagagtcagtgctacaagctgctggaattgagctaggatcgaactatcctcttccaattgtgggaatagatgcagcaaaaaacagattactagaagcactatcaaaaatgtggcaacaagaggcagcttcaagagctgcaatggaaaatggaacggaggaaggacttggagactcttctgaatcagttcctgctgcttttcctcaagacacgcgaatggaggaaactcatgaacctgttaggaacaatccccttcccattgctcggcgctaccaagatcagatggttccaagcatcacttcttccttgttgagagtggaagaggaagaaacgtcttctgatcttcgaaactcagcagaagagagcagcagagctgaagtgccagtaactgcaaatgcacaacaaaatgcaggagttgcattgaacgaacggatgttgcagaccactaataggaatacacaaacccaatacaatactacgatggacctgagaaatgtagctgatgattctgcagtagaatcttcaagtggtaccaggagagaaagggatggaggtttagttccagtatggtctccaccagcttccagttactcagaacaatttgtaggagatgaaaatggtataacaagcagttcatcttacttgcaaaggcatcctcaatctcaccaaatgctgaattggaagcagctacctcaaactgggtaaatgggtagcaacaggactattgtttggtttagaagggaccttagaattgaggacaatcctgcattaactgctgctgcaaaggagggttctgtactccctgtgtatgtatggtgccctaaagaggaaggacaattttatcctggaagagtgtcaaggtggtggctgaagcagtctcttgctcacttggatcaatcactgaagtctcttggatccagacttgtgctcatcaaaacccacagtactgctgtggctctcgtggaatgcgtaaaggccattcaagcaaccaaagtagtgtttaaccatctgtatgatccagtttcacttgtccgtgatcacaacatcaaagaaaagctagtggaacaaggcatatctgttcaaagctacaatggagatctgttgtatgagccatgggaagtaaatagtgagagtggacgtgcttttactaccttcaatgctttttggaagaaatgcttgcacatgcaaatggatattgtttcagttgttcctccatggcaattaattccagctgaaggaaaaattgaggaatgttcattggaggaacttggtcttgaaaatgaatcagaaaaaccaagtaatgcattgctaggacgggcatggtcaccgggttggagaaatgctgacaaggctttaagagaatttgtggagcttcatctacttcactactccaagaaaaggctgaaggttggtggagagtccacctcactcttgtccccatatcttcattttggagaattaagtgcaaggaaagtttttcaagtgacttgtatgaagcaaatattatggacaaatgaaggaaacagtgctggtgaagagagtgcaaatcttttcctcagggccattggacttagggagtactcacgctatctttgtttcaactttcccttcacccatgagagagcacttctggggcacttaaagttttttccttggaatcctgatcctgatatctttaagacttggagacaaggtaggactggctttcctttggttgatgcaggaatgagagaactttgggcaacaggatggatacacaacagaataagagtaatagtttccagttttgctgtgaaaatgttgcttctaccctggaaatggggaatgaaatatttctgggacacacttctggatgcagaccttgaaagtgatatcttgggttggcaatatatctcaggaggcttaccagatggccatgagcttgagcgcttggacaatcctgagattcaaggggctaaatttgatccagaaggtgaatatgtgaggcaatggctacctgagttggcaagaatgccaactgagtggatccatcatccttgggatgcaccacttactgtgcttcgagcagcaggagttgagctgggccagaattacccaaagccaatcattgatatagatttggccagagaaagactcactgaagctatattcaagatgtgggaatctgaagcagcagcaaaagctgctggctctgaaccaagggatgaagttgttgtagacaactctcacactgttgaaaatttggacactcaaaaagttgtcgtcctgggaaaggctccatgtgctactatttcagctaatgatcaaaaggtgcctgctcttcaggattccaagaatgaaccgcctacgcggaagaggccaaagcacatgatagaggaggggcagaaccaggatcactctcaaaatcataacaaagatactggcttgtcaagcattgaccaagacatttgctccacagctgattcttcatcatgtaagaagcagtgtgccagtacaagctcatattcattttcagttccacagcagtgttcctcatcttctaatctgaagtggccatggcaagagaagattgatatggagcagagttcaagcaaagatggtaagtacattcttacatttccaagggatttatga

SEQ ID NO: 21 - DNA, 1917 bp, Glycine max
  source 1..1917: mol_type = genomic DNA
atggcaaactttccagttgttgacatggggaagcttaacaccgaagagagacctgcagccatggagattataaaagatgcctgtgagaactggggtttctttgagttggtgaaccatgggatatccattgagcttatggacacagtggagaagctcacaaaagagcattacaagaagactatggagcaaaggttcaaagaaatggtgaccagcaaaggtcttgagtcagttcagtcagaaatcaatgacttggactgggaaagcacctttttcctgcgccatcttccactctccaacgtttcagacaacgcagatcttgatcaagattacaggaagacaatgaagaaatttgctctggaactggagaaacttgcagagcagcttctagacttgctatgcgagaatctgggcctggagaaagggtacctgaagaaggtgttctacggatccaagggtccaaattttgggacaaaagtgagcaactaccctccttgtcccacccctgatctgatcaagggactaagagctcacactgatgctggtggcataattctactcttccaagatgacaaggtgagtggcttgcagctcctcaaggatgaccagtggatcgatgtcccaccaatgcgtcactccattgtaatcaaccttggtgaccaacttgaggtcataaccaatggcaagtacaagagtgtgatgcaccgagtgattgctcaaaccgatggtaccagaatgtccatagcttctttctacaaccctggtgatgatgctgtcatttctcctgcaccagccttggtgaaggaattagatgagacaagccaagtgtacccaaaatttgtgtttgatgattacatgaagctctatgctggtctcaagtttcaggctaaagagccaaggtttgaagctatgaaggccaatgcaagcgtagttgatgtgggggccatagccacagtttgaatggagaactttccagttgttgacatgggaaacctgaataatgaagagagatcagcaaccatggagataatcaaagatgcctgtgagaactggggtttctttgagttggtgaaccatgggatatccatagagttgatgatggacaccgtggagaggatgacaaaagagcactacaaaaagtgtatggagcaaaggttccaagaaatggtagcaagcaaaggcttggagtctgctcaatctgaaatcaatgatttggactgggaaagcactttctttttgcgccatcttcctgtctctaacatctcagagatccctgatcttgatgaagattacaggaaggtaatgaaggattttgccgtggaactggagaaactggcggagctagttcttgagttgctgtgtgagaatcttgggctagagaaagggtatctgaagaaggtgttctgtgggtcaaagggaccaaattttggaacaaaagtgagcaactaccctccatgtccgaagccagagctgataaaggggctgagagcccacacagatgctggtggcatcattctactcttccaagatcacaaggtcagtggattgcagctcctcaaggatgcccactggattgatgtccccccaatgcgccactccattgtcatcaaccttggagatcaacttgaggtcatcaccaatggcaaatacaagagtgtgatgcaccgtgtaattactcaaacggatggtaatagaatgtccatagcctcgttttacaatccaggcaatgatgcattgatcgctccagcaccggccttggtgaaggaagatgagactagccaagtttatcctaagtttgtttttgatgattacatgaagctctatgctggccttaaattccaggacaaagagccaagatttgaagctatgaaggccacggagtcatccaacattaatttagggcccatagcaacggtctaa

SEQ ID NO: 22 - DNA, 4494 bp, Glycine max
  source 1..4494: mol_type = genomic DNA
atgatggaaatgcctgccagaagatccaactactcgctcctcagccaaattcccgacgaccagttctccgcctccgccgccccctcctcctccggcgacggcaagtccgcccgcgccggtaagtccgaccgcgccgccttcgactgggacctcgtcgccgatcaccgggccgccaaccggatcggaaacctctactcctcgatcggcctgcagcgccagtccagcgggagcagctacggcgagagctcgctctccggcggcggcgacttttatgctccgacgctctccacggcggcggtcagcgacgtcgacgccttcggatacctccacgacgagcggagcaagttctcggaggtggcgccggcgaggatcgccggctcctcctccgggaagagctgggcgcagcagacggaggagagctaccagctgcagcttgcgctcgcactgcgcctctcgttgcacgccacgtgcgccgatgatcccaatttcctcgatccggtgccggacgacgccgcgccgcggctctcgtcgtcggccgaggcggtttcgcataggttttgggtgaatggctgcctatcatactctgacaaaattccagatggcttttacctgattcatgggatgaattcctttgtctggaccttgtgcactgatctgcatgaaaatggccgaattccatcagttgatatgctgaagtctgtgaatccctgcgtcgtttcttcacttgaagtagttatggtggatcgacgcagtgaccccagcttaagagatctgcaaaataatgttcataacatttcttgtactagcataacaacaacagatgttgtagataaactttccaagctggtttgcaaccgtatgggtggttcagcttctgttggggaagatcactttttttccatctggagggattgcagtaatgatctgaaagattgcttaggatctgttgttattcccataggtagtctatctgttggcctctgccggcatcgtgctatattattcaaagtactagctgacgccattgatttaccatgtcgaattgctaagggctgtaaatattgcaaaagggatgatgctacatcttgtcttgttcgatttgggcttgagagggagtatcttgttgatttaattggaaagccaggaaacttatctgagcctgattccttgctcaatggtccatcttccatctcattttcttcacccttgcgctttccacgacttaaaccggctgaacctaccattgatttcaggtcattggccaaacagtatttctcggattgtgtgtctcttgagcttgtcttcgacaacaactctgcagaacagtttgatgggaagtgcaaagacagcagtaaccctaggccaatttcgtctgatagcaatagaagttctcaccttcctctgcatccgcaagattctcatccaagctcacgtgaacaaggttctgaaacatatcaatcatgtaacgctcctcagaacattgtagactcaactctgggaaagtatccaccaccaataaagcataagcgtcctgctggtataccaaccccattagcacttacaaatactaatgatgacatgattgagggcaagaggtttgttgaaggaagtcaactgattcctagcaaacatgctcgagagcttaactttgacatggaggatttggacataccatggagtgatcttgtcctaagagagaaaatagggtcaggttcttttggaactgtacatcgtgctgagtggaatggctcggatgttgctgtgaaaattttgatggaacaggattttcatgctgagcgcttcaaggaattcctaagggaggttgcaataatgaaacgtttacggcatccaaacattgttttgtttatgggggcagtcactcagcctcctaacttatcaattgtcacggaatatttatctaggggtagcttatataggcttttgcatagatctggcgccaaagaagtattggatgagaggcgtaggcttggtatggcttatgacgtggcaaaggggatgaattatcttcataaacgtaatccccccattgttcatagagatctgaaatctccaaaccttcttgttgacaagaaatatacagtgaaggtttgtgattttgggctttcacgtctgaaggccaatacgtttctctcatccaagtcagctgctgggactcctgagtggatggctccagaggttctttgtgatgagccatcgaatgagaagtccgatgtttacagctttggtgtaatcttatgggagcttgcaacattgcaacagccatgggttaatttaaatccagcacaggttgtggcagctgttggctttaagagaaaaaggcttgagatcccacatgatgtgaatccccaagtagctgcgctaattgaggcttgctgggcctatgagccttggaaacgtccttcttttgcgagtattatggattctttgaggccattgctcaaaccccctacacctcaacctggtcgtccaagcatgccattactgacctgaatgtacagcagtggtggcgctgaagccgaatcggaatctgaatctgagcgcaatagaatgggtgatgataaggaagaacagggctcctcgttcaagagctgggccaaacaaacggaggagagttatcagctgcagctcgctcttgctcttcgtctttcttctcattcagcttcttcttcggatcacccctcttcttcggcccagacgctcacgcaccggttttgggtagatggttgtttgcaatactctgataaaattatagatggattctacttaatccatgggatggatgtatatacatggactataagtactgatctgcagaatgttggtatgattccgtcatttgaatcactgatgtcaattgaacctagcgatgacttgtcaatcctagtggttgctgttgataaatctagagatcctggtttgagagaactgcaaaatagggtagcaagtctttcaaataactggattaccacgaaagatgcaactgatcagcttgcaaatcttgtttgcaataggatggggggaggatctttaactgaggaaaatttaggtacacgctggaaggaatgcacccaacttcttaaaagctgcctgcattccgtcattcttccaattggaagcttacccattggcctttgtgtccatcgagctttgctgtttaaagtgttggctgacttaatcaatctaccatgccgtattgcaaagggctgcaagtattgtaggaaggatgtgggtgcctcatgtatagtgcaatttggttctgacagggagtatatgattgatttagttggaagacctggggcaacatgccagcctgattcttcactgaattctgcatcttcaatgttggtcccttcaccattatgtcatccaaagttcaagccagttgaaacagctgaatatacaaagacactagcccaattgtatttcttggacagtgaagcactgcatcttgtatttgataccacatcaggacctgctgttaatcatagtggcagaatggatctacagaaaactgaagctctgggtgcaaattatgcgggtgggaacagccatctgattgctttgattcctggtgcagaggaatatgagtctttcaatgaagcaaaccagtcaataatggattacccaagtcatgaagttgatcttgacaaggaggatctggatattccatggagtgaacttattttgaaggagaacattgggacagggtcatttggaaccgtccttcgtgcggactggcgcggttctgatgttgctgtgaagattcttaaagtgcagggttttgatcctgggcgatttgaagaatttctaaaggaggtctcactcatgaagcgcctccggcatccaaacatagtacttttgatgggtgcagttattcaacccccgaagttatcaatagtaacagaatatttatcaagaggcagtttgtatgaacttctgcacatgcccaatgttggatcatcacttagtgaaaagcgtcgtttaagtatggcttatgatgttgcaagcggaatgaattatcttcatcaaatgagacctcctattgttcatagagatttgaagtctccaaatcttttggttgatgattcatacactgttaaggtatgtgattttggcctttcacgtacaaaggcaaacacatttctttcatctaaaaccgctgccggaactcctgaatggatggcacctgaagtcattcgaggagaactgtcaagcgaaaaatgtgatgtattcagctttggtgtaatcttgtgggaactagtgaccctgcaacagccatggagacaattaaatccttcacaggttgttgcagctgttggttttatgggcaaaaggctagagattccagggcatgtgaatcctcaagtggctgccttgattgagctctgctgggccacggaacactggagacgaccttctttctcctatgttatgaaatgtttgcagcaaataattgctgatgctaaagggtga

SEQ ID NO: 23 - DNA, 1611 bp, Glycine max
  source 1..1611: mol_type = genomic DNA
atcctatttttttcatatacaacaaacacggattcattgcatcaccgtcaccaccactttcatcatcataaaacatttttcatataaataaaatatattcattcattcacacatcgtaataatcattatctatcattctctctctttcaattttagacagaagactaaaagagtgaagagaacaaacagagcaatggaaggaggaggagctgctccgccggccgacaccgtgatgtccgacgcggcgcctccgccgcagcaagcgatggctatggggatcgagaatattccggcgacgctgagccacggtggcaggttcatccaatacaacatattcggaaacatattcgaagtcaccgccaaatacaagccacccatcatgcccatcggaaaaggcgcatacggcatcgtttgctcggctttgaactcggagacgaatgagcacgttgccatcaagaagattgcgaatgcgtttgataacaagattgatgcaaagaggaccctccgtgaaatcaagctgcttcgtcacatggatcatgaaaacgtggttgcaatcagggatatagtgccaccacctcaaagggagatattcaatgatgtttacattgcatatgagttgatggacactgaccttcaccaaattattcgttcaaaccaaggattatcagaggagcactgtcagtattttctgtatcaaatcctccgtgggttgaagtacatacattctgccaatgttctgcatagggacttaaaacctagcaaccttctcctgaatgccaactgtgacttaaaaatttgtgattttggactggctcgtgtcacgtctgaaactgattttatgactgaatatgttgttacaagatggtaccgtgcaccagagcttctgttgaactcttctgattacactgcagcaattgatgtatggtctgttggttgtattttcatggaactgatggatcgcaagcctttgtttcctggcagagatcatgtgcatcagttgcgtctacttatggagctgattggcaccccatcagaggctgatttggggttcctgaatgaaaatgctaagagatacattaggcaactacccctttaccgccgtcaatctttccaggaaaagtttccacatgtccatcctgaagctatagatcttgttgaaaaaatgttaacttttgatcctagaaaaaggattactgttgaagatgcactggcgcacccgtacttgacatctctgcatgacatcagtgatgaaccagtgtgcatgactcccttcaactttgactttgaacaacatgctttgacggaggaacagatgaaagaactgatataccgagaggctctagcatttaaccctgagtatcagcagtagtagtgatagtcttgttgtcattggatttggatcgtgtgatgtttgtgtccatagatcaagggatttcattgtgtatatatatctatcatctgaaaccagatgatcttggagttgaattatttccatcagagacatgattggtagactggaacaatcgtaaggtatttgtattggcttatttgcttcatattattattgtccaattcattggctccagccaactgataaatcttttcttggtc

SEQ ID NO: 24 - DNA, 1440 bp, Glycine max
  source 1..1440: mol_type = genomic DNA
cttggtctttgaattctgtggaggagcagcttcttgggttgggttctctgagtttttttacctcttcttggtttgatccagaaaggcttatttttctttgttctgaggaaaaaaattctgcttttcaatttctgggttttcaagcttcgtgtagaaggaaatggcacaacgttctcatgtacccaaatttggcaattgggatagtggagagaatgttccttatacagcatattttgacaaggcacggaaaggtcgaactggcacaaggataataaatccaaatgaccccgaggaaaatgctgatttaagttttgataatccatcatctgataacctacctcctaccagacctagaactaattcagaggatcaatccggaaaaggatcactgcatttggaagatgatcccaagaattttatcgaatctccagctcgtcatgacaatgtaagcagtaggagtggcagtagaagccatggagtaggttctgctgataaccgtagaagacattcaacgcaaagtactgggtctgagtacagcattgaacgttcaccgcttcatcgccaggctagagccccaggcagagacagtccccagtgggaaccaaagaattcatatgacagcagtcagggaacaccaggaaggtctcggttaagaccggctaatcgaggagatgaaacaccagataaaggtgcagctgttccaaagtttggtgactgggatgtgaataaccctgcatcagcagatggctttactcacattttcaacaaagtgagggaggagagacaaggcgggccaggacaagtgccaggaacgcctaatgagagaccacaacctattaatggcctatctaatgatgacaaagtccagtgttgctgctttgcgtggggtgggaaaaaatgatttcttggattggaagtctgaatgataatattctgaacagaaatgatggtaaagataatgttgtgaatggtcatgaggtgtgtgtagctcttgtaaatatccaaggctctctctctggtcgggaagacatcgttgaagcaaatgtatcgcaactactctgccgtttcttctcccttttgtttttctttctaatataaccaaagattctatgcgtgcaagattgtgatttttctttgtattttcttctttttgagccttctgttgtgttactgttatgctttacaattcaaattaactgtatgtacctgagatatagagggagagtggttatataattcttttccaatgaattggtgggttgtgaatcgaggtctattgtgttcatgttggtgttgaacttctctagtttaactgggaatgatctggtagcaaattgtttcaaaaagtgatattatcatggcctgtttgtttcagttaaaatagatgttatgttattaagtaagctcatattctatttgaattggtcacttttttaccat

SEQ ID NO: 25 - DNA, 1485 bp, Glycine max
  source 1..1485: mol_type = genomic DNA
agaaacaggatcggaggtagagtaatttcatatatattactttgaaaaaatcttaatgctatgtacgactttgaagggttaaagagagagatattttgaaacagagaagaaaaacagagaaattacagacaggaaaaataatagtcaaacattaccggtggcgccggtcaacggttgataaacctctctctctctaaagctcttctttatgtctctctctcttgcttggtctttgaattctgtggaggagcttcttgggttggttctctgagtttttacctcctctgggtctgatccaaaaaggcttaattttctttgttccgaggaaaaaattctgcttttgaatttgttggttttcaagcttggtgtagaaggaaatggcacaacgttctcatgtacccaaatttggcaattgggatagtggagagaatgttccttatacagcatattttgacaaggcgcggaaaggtcgaactggggcaaggataataaatccaaatgaccctgaggaaaatgctgatttaagtctggataatccatcatctgatcacctacctcctaccagacccagagctaattcagaggatcaatccggaaaaggatcattgcctttggaagatgatcctaagcattttgtcgactctccagctcgtcatgacaatgtaagcagtaggagtggcagtagaagccacggagtaggttctgctgaaaaccgtagaagacattcgacacaaagtactggttccgagtacagtattgaacgttcaccacttcatcgccaggctagagcccctggcagagacagtccccagtgggaaccaaagaattcatatgacaacagccagggaacacctgggagatctcggttaagaccggttaatcgaggagatgaaacaccggataaaggtgcagctgttccaaagtttggtgactgggatgtgaataacccatcatcagcagatggctttactcacattttcaacaaagtgagggaggagagacagggtgtcccaggacaagtgccgggaacgcctaatgagagaccacaagctattaggggccaatctaacgatgacaaagtccagtgttgctgcttcgcatgggggggcaaaaaatgatttcttggattagaagtgtgaatgataatatttgaacaaaaatgatggaaaagatagcgttgtgaatgatcatgaggtgtgtgtagctcttgtaaatatccaaggctctctctccaagcgggaagacatcgatgaagtaaattgactgcaactactttgttccttttcttctcccttttgtttttctttcaaatattcaaggagactctttggatgcaagattgtgctttttctttgttttttcttcattttgagccttctgttgtgttactgttatgctttacaattcaaattaactgtatttacctgagatatagagggagattggttatataattattttccaatgaattgatgggttgtgact

SEQ ID NO: 26 - DNA, 4945 bp, Glycine max
  source 1..4945: mol_type = genomic DNA
attttattttatttttctcgtttcgcttcacaaaatcgcaatttcgtatcactcgtttccgttttcccttcaggtagagacagtgcctgccgccactgttctatgcgccccccttcatcatcatcaccatccctaccatcatcaatgccaacaccataaccatcatcatcatcatcaccatcaccttcatatccctcactctgcgaaaacctagggttcggtcgtcaataccctccctcccccctattattattccatgtacgatcaccactgaggagggtttctgatttggatggcattgggagatttgatggcctctcacttatcccaatccaccgttcttgtcgttccctccatccacagccacctcgacgattccaccacctccgccgccgtcgccgctgttaacaacagcagtagcaacgacgacgccgattttgcgcaccgccgtgactccgaagctgccatctccagcagcagcagcggcaactatgccggtaatgccgccaccagcatggcctaccttccccacaccgttttcctctgcgagctccgccacgacgccttcgaggccgccgtccccgctggcccctccgacagcggcctcgtctccaagtggcggcctaaggacagaatgaagactggatgtgtagctctagtattatgtttaaacattagtgtcgatcctccagatgtaataaagatatccccttgtgccagaatggagtgctggatagatcctttttctatggcaccacaaaaggcattggagtcaattggaaaaactttgagcagtcagtatgaaagatggcaaccaaaggcccgctataaatgtcaacttgatccgacagtggatgaggtgaagaagctttgtactacttgtcgtaaatatgcaaagtcggaaagagttttattccattacaatgggcatggtgtgccaaaaccaactgctaatggtgaaatttgggtcttcaataagagttatacacagtatattcccttacctatcagtgaacttgattcctggctgaagaccccatcaatttacgtcattgattgctctgcggctgggatgattgtgaatttcttcattgagcttcatgaatggagtccttccaactcctctgtgtcccaaagggattgcattctgcttgcagcatgtgaagcacatgagactttgcctcagagtgcagaattcccagctgatgtgtttacatcttgccttaccacacctataaagatggcattgcgatggttttgtactcgttcattacttcgtgaatcacttgttaattcacttatagacaagatccctggccgtccaaatgaccgaaagacacttctgggtgaattgaattggatctttacagcagtaactgatacgattgcctggaatgttcttcctcatgatctttttcagagactgttcagacaggatttgcttgttgcaagtctgtttcgaaattttctacttgctgagcgaatcatgcgatctgcaaattgttctcctgtctctcacccaatgttacctccgacccatcagcatcatatgtgggatgcatgggacatggctgctgagctctgcctctctcaacttccatctttagttgaggatcctaattctgaatttcagcgtagtacatttttcaccgagcagttgacagcatttgaggtatggcttgaccatggttctgaacataagaaaccaccagagcagttgcctatagttcttcaggttttacatagtcaatgccatcggtttcgggctttagtactccttggaaggttccttgatatggggccatgggctgtagatctggcattatctgttggaatattcccatatgttctaaagctgttacaaacaacaacaccagaactacgacagatccttgtattcatatggacaaagattcttgcacttgataagtcatgtcaggttgatctagtgaaggatggtggtcatatctattttattaagtttcttgacagcatggaagcatatccagagcaacgcgcaatggctgcttttgttttggctgtgattgtggatggtcatagacgaggccaagaagcttgtattgaagctggtttgattcatgtctgcttaaagcatcttcagagttcatgtcctaatgattcacaaactgaaccccttttccttcagtggctttgcctgtgtttggggaaattgtgggaagacttttcagaggcacaaacaattggtttgcaggaagatgccactactatatttgctcctctactgtctgaaccccagccagaggttagggcttctgctgtttttgcactaggtaccattcttgatgtgggatttgattcatgtagaagtgttggtggagatgaagaatgtgatgatgatgataagtttagggctgaagttagtatagttaagagtatgttaggtgttgcttcagatggtagtccattggtgagggcagaggtagcagtggctctagcacgatttgcttttggccacaacaagcacctgaaatctattgctgctgcatattggaagcctcaagctaattctttgattaattccttaccttcattggctaatataaaaggttcggttggtggatatgctaaacagaaccaacacatgccttatggaagtatcgtttctcctcaaattggtcctattagggttggaaatgacaattctccagtgattcgagatggccgcgtgtcttcaagcagtcctctcgctggttcaggaatcatgcatggatccccattgtctgatgattcatctcatcattctgattcaggaattctgaatgatggtttcagcaatggagtggttaaccacactggaccaaagcccttggacaatgcattgtattcgcaatgtgtacttgctatgtgtactttagcaaaggatccttctccacgcattgcaaatcttggtcgccgggtactgtccataataggtattgaacaagtggttgcaaaaccattgaagtttagtggtgttcggactgctgaatctacagcttctccactggctcgttcatcttcttggtttgatatgaatggaggacacttgccactgaccttcagaactcctccagtcagtcctcctcgcccaagttatataactcgaatgcgtagagtttgttcattggagtttaggccccacctgatggattctcctgactcaggattagctgatccactcttaggttctggtggagcttctgggacttcagatcgaagctttcttccacaatcaactatatatagttggagttgtgggcacttttccaaaccacttttaactgctgcagatgatagcgaagaggcatcagccagaagagaagagagggaaaaatttgcattggagcacataggcaaatgccagcactctgctgttagcaggctaataaatccaattgctaaatgggacataaagggtacacaaacagcattgctgcaacctttctctcctatagtgatagctgcagatgagaatgaacgtatcaggatatggaaccatgaggaggctacactgcttaacagttttgataatcatgattttcccgacaaaggaatttctaagctctgccttgtaaatgagcttgatgatagcttgcttcttgctgcttcatctgatggaaacattcggatttggaaggactatactcttaagggtaaacaaaaacttgtcactgcattctcttcaattcatggtcataaacctggtgtgaggagtctgaatgcagttgttgattggcaacaacaatgtggctatctgtatgcatcgggtgagatatcatctattatgctatgggatgttgataaagagcagcttgttaattctaaatcttcatcatcagattgcagtgtctcagtgttggcagcatcacaagttcatgggggacaatttgcagctggtttcgtggatggttctgttcgactttatgatgtcagaacacctgatatgctcgtctgtggattaaggcctcatacacaaagagtagaaaaggttgtggggattggctttcagcctggattagaccaaggaaagattgttagcgcttcccaggctggggatatccaatttcttgatataagaaatcatagcagtgcgtatcttactattgaagctcataggggttcactcacagctttagcggtacatagacatgctccaattattgccagtggttcagcgaaacaattcattaaggtctttagtctggagggtgatcaactaggcaccattaaatactatcctaccctaatggcccagaaaattggttctgtaagctgcctcaatttccatccataccaagtattacttgctgctggtgctgcagatgcatgcgtttgtatttatgctgatgacaacactcaagcaagatgaatccacattttttccaatgatttcatcggttttctccagctcagaagagtgcatcaaacatctctcaggattatcatgcatatttcttgtagttggataatcctgtgatgatgctttaatccctctccgctgtgaatacatctgaaaatattggtagggtacattctgctgatagtgtatgcaagatattcattagcctccatgcttagagggttgcactcgacaggaagaaagtcatgcctattgattttggaggcttcggcaaataatgtgaaaattcgtaaatgtgtgtaaatatcattttctccataattatttttcattcctgttttgcgtgctattcccgcccttgatttgtaaattttttagtcagaatacataagctagtttgctgacaaatatgggtgattggctcctgaaaatgggccatgtcactcatgattgtagtttctgttgaatcgggggacccttgtttttaaccaacgtgtaattatatattcaagtttgtctaagtgtggaatttctgtctcaatctaaaggtatagttttaatggg

SEQ ID NO: 27 - DNA, 7982 bp, Glycine max
  source 1..7982: mol_type = genomic DNA
acgtccatcccgaattaagtaggcatggacatggcgaaacgcgatttccattttcaatttgagtttctacttgtcctttccaaattcctatcgcaactaaactaatttagggcttaggttagcctagccaaataaataaattaatggcaaccgcgtctcagtcccaccgttacatcggccctccctccgtcgcacccggtcctggcgacgctctcaaccgcatcctcgccgacctctgcacccgcggcaatcccaaggagggtgcttctcttgctctgaagaagcatctagaggaagaagctcgtgatattagtggcgaagccttttcgcgttttatggatcagttgtatgacaggatctctggccttctggacagcagcgacgttgccgagaatctgggagctctgagggctattgacgagttgattgatgtggcgctcggggagaatgcttctaaggtctccagattttccagctacatgcgtattgtctttgacaccaaacgcgatcccgagattctcgttcttgctagtagggttctcggtcaccttgctagggctggtggtgctatgactgccgatgaagtcgaacgccaggttaaaattgctctcgattggcttcgaggaaatagagtcgagtatcgtcgatttgctgctgtcttaatcttgaaagaaatggcagaaaatgcttccactgttttcaatgtccatgtgccggagtttgtcgatgctatctgggttgctttgagagatccagtgttgccagtccgtgagcgggctgtggaggcattgcgagcttgccttcgtgttattgaaaagcgtgagacacgatggcgcgtgcagtggtactatcgaatgtttgaagcaacgcaagatggattgggtaaaaatgcacctgtgcacagcatacatggttcattgcttgccgttggagagcttttgaggaataccggtgagttcatgatgtcaagataccgcgaagttgccgaaattgttctcagataccttgagcaccgggatcggcttgttcgcctgagcataacttcattgctgcctcgaattgcccattttctacgtgatcgttttgtcactaactacttaactatatgcatggatcatattctttctgtcttaaaagtaccacaagatcgtgatagtgggttcatcgctctcggagagatggctggggcactggatggggaactcatccattacttgccgacaataactactcacttacgtgaggcgattgcaccacgaaggatcaaaccttcacttgaggccttggcttgtgttggaagtattgctaaagcaatggggtcagcaatggaacctcatgtacgtggacttttggatattatgttttctactggcctttctactgtacttgttgaagcccttgagcaaataagtaccagcattccgtctctgctgccaactattcaagaccggttactagacagcatatctatggttctttctaaatcacattatcatctggggaggcctgcacaaagcgtgggcagaggaacaataataaatgtccctcaacaagtttcagagcttaatggttctgctcttatacaacttgctttgcagactcttgcccgttttaactttaagggccacgaactacttgaatttgctagagaatcagttgttgtctatttggatgatgaggatggagctacacgaaaagatgcagctctatgttgttgcagattgattgctagttctttctctggtatggcatgctcacattttggttctagcaggttgactcgatcaggggggaagcgccggcgccttgtcgaggagcttgtagagaagcttctcatttctgctgttgcagatgctgatgttactgttcggcattctatctttacttctctccatggagatagaggttttgatgagtatttggcccaggcggacaacctgagtgcagtatttgctgctttaaatgatgaggattttgatgttagagagtatgcaatttctgtggctggaaggctatcagagaaaaatcctgcatatgttcttccagctcttcgtcgccatcttatacagttgttaacttacttagagcagagtgcagatagcaaatgtaaagaagaaagtgcaaagttaattggctgtttgataaggaattgtgaacggctaataattccatacattgctccaattcataaggcactcgtagccagacttattgatgttaatgccaatactggaaccattagtggagtccttgtaaccgtgggtgatcttgcgagggtgggcggttttgcaatgaggcaatacattcctgaacttatgcctttaattgttgaagctttattggatggagctgctgtctccaaacgtgaagtggctgttgctacactaggtcaagttgtgcagagcacggggtatgttataactccatataatgagtatcctcaattacttgggttactcttaaaattactaaatggtgaactagtatggtccaccagacgtgaagtattgaaggttcttggcattatgggtgctctggatcctcatttgcataaaagaaatcaaaaaaccttgccagggcctcatggagatgttacccgtcctgctagcgattcaagtcaacagattcagtctatggatgaatttcccttggacctttggccttcttttgcttcatcagatgactattactccacagttgcgattaattctctcatgcgaatattgagggatccatcacttgctagttatcacttaaaggtggttgggtctcttatgttcatattcaagtcgatgggacttggctgcgttccatacctgccaaaggttttgcctgatctttttcatactgttcggacatgtgaagatagtttaaaggactttataacatggaagcttggaacactggtgtctattgtgcgccagcacattcgtaaatatttgcaagatttgctatctttaatatctgaattttggtcagccttcactcttccagcaccagctcgtcctggtcctggctatccagttcttcacctggtggagcagctttgcttggctcttaatgatgaatttagaacatatcttccagttattctgccaggttgtattcaggtccttagtgatgcagaacggtgcaatgactacacttatgttcttgatatccttcataccctagaagtgtttggtgggactttggatgagcatatgcatctacttcttcctgctctaatacgattgtttaaagtggatgcctcagtagacataagacgtgctgcaattaaaactttgacaagcctgatccctcgtgttcaggtcactggtcatatatcttctcttgtgcaccacttgaagttagttttggatgggaaaaatgatgagctccgaaaggatgctgttgatgcactttgttgtttggctcatgcgcttggcgaggatttcacaatttttattccatcaattcacaaacttctacaaaaatatcgcttgcggcacaaggaatttgaggaaattgaagggcgtttacagcgacgggagccacttattcttgggatcacagcttcacaaagactgaacagacggcttcctgttgaggtcattagtgatcctttagatgatgtggagattgatccttatgaagatggatctgatgctcacaaacttagagaccatcaggttaatgatggtcgattacggacagctggggaggcttctcagcgaagtactaaggaagattgggcagaatggatgaggcattttagcattcaacttctaaaggagtctccttccccagccttacgaacctgtgctaggcttgcacagttgcagccttttgttgggcgagagttgtttgcagctggatttgtcagttgttgggcgcagctaaacgaaactagccagaaacagctagttcagaatctagagatggcattttcttctccaaatatccccccagaaattttggcaacacttcttaacttggctgagttcatggagcatgatgagaaacctcttcctattgacatacgccttcttggtgctcttgctgagaagtgtcgtgcatttgcaaaagccttgcactataaagaaatggaatttgaaggagcacgttccaagaagatggatgctaacccagttgctgtagttgaagttcttattcatataaacagccaattacaccaacatgaggctgcacttggaatactaacctatgcccaacagcatctagattttcaacttaaggagtcatggtatgagaaactgcagagatgggatgatgccctcaaggcttatacagcaaaggcttctcaagcaacaagtccacatcttgtcttggatgccactttaggtaaaatgagatgccttgctgcattggctcaatgggatgagttgaatatcctatgtaaagaattttggacccctgcagagccagctgctcgtcttgaaatggcaccaatggctgcaagtgctgcttggaacatgggagaatgggaccaaatggctgaatatgtgtctcgattggatgatggtgatgaaacaaaacttcgaggtttgggaaataccgctgccagtagtgatggaagtagtagtggtactttcttcagggctgttctattggttcgtagaggaaagtatgatgaagcacgtgagtatgtagaaagagctagaaaatgtctggcaacagagcttgctgctttggttttggagagttatgaacgtgcttatagtaatatggttcgtgttcagcaactatcagaactagaagaggtgattgattaccgtacgcttcctattggaaaccgagtagcagacgagcgacgggctcttattcgcaatatgtggactcagcgtatagaaggagccaaaagtaatgttgaggtttggcaggcacttttagctgtcagagcacttgtgctgcctcctgtggaagatgttgaatcctggctcaaattcgcctctctttgcagaaaaagtgggcgcattagccaagcaaaatctactttagttaaacttctacagtacgaccctgaaaagtctcccgaaaatgttcgataccatggccctcctcaagtaatgctggcatacttgaagtaccagtggtcacttggagaggattcaaagcgcagggaagcatttataaggcttcagaatttggctatggaactctcaagtgcacccaacattcaaccagttacaccatcaagcttcacaaatggtttgaatcttagtgttcctctccttgctcgtgtttatctaaatcttggttcctggcagtggtcactttctcctgggctagtcgatgaatctattaaagatatacttaatgcattcacaaaagctactcagtatgccaataaatgggctaaagcttggcataaatgggcattattcaacacagcagtgatgtctcattacactttaagaggtttccccgatgttgcagctcaatttgttgctgcagctgtcactggatactttcattctatagcatgtgcagcaaattccaaaggtgttgatgatagtttacaggatattcttcgacttttgacattatggttcaaccatggagctactgcagaagtacaaatggccctaaagaaaggattttccctggttaatatcaatacttggctagtagttctacctcaaataattgccaggatacactctaataaccatgctgttagagagttgatacagtcacttttggttcggattgggcaaaatcatccacaggctctcatgtatcctttgcttgtggcctgtaaatcaataagcaacttacgcaaagctgcagctcaggaagttgttgacaaagttcggcagcatagtggtgtactagtggatcaggcgcaacttgtatcgaaggaactgatcagagttgctattctttggcatgaaatgtggcatgaagcactggaagaagctagccgtttgtattttggtgagcataatattgaagggatgctaaatgtgttggagccattgcatgaaatgcttgaagaaggagcaatgaaaaacaatgtcaccataaaggagagaatatttattgaggcttatcgacaggaattattagaagcttatgagtgctgtatgaattataagagaactgggaaagatgctgagctgacccaggcttgggatatttattatcatgttttcagaaagatagataagcaacttcaaagtcttacaactttagacttggagtctgtttctccagaattgctagaatgccgcaatttggagcttgctgttcccgggtcataccgtgctgatgcaccagtggttacaattgcatcatttgcacgccaacttgttgtcataacatccaagcaacgacccaggaagttaaccattcatgggagtgatggtgatgattatgccttcttattgaagggacatgaggatttacgccaggatgaacgagtcatgcagctttttggtttggtgaatactctccttgagaattctcccaaaacagcagagaaagatctgtctattcagcgatatgctgtaatccctttgtctccaaacagtggtttaattgaatgggtcccaaattgcgacacattgcatcatcttattcgggagtatagagatgccagaaagatcacattgaatcaggaacacaaatgcatgctcagttttgcacctgattatgatcatttgcctcttatagctaaagtggaggtgtttgaacatgcgttgcacaatacagaaggaaatgacctagcaagggtactttggttgaaaagtcgaacatctgagatatggcttgagaggaggacaaattatacaaggagtttggcagttatgagcatggttggttaccttcttggtttaggtgatcgccatccgagcaatcttatgttacaccggtttagtgggaagattttacacatagattttggagactgttttgaagcatcaatgaaccgtgagaagttcccggagaaggttccttttcgcttgacaagaatgcttgtgaaagccatggaagttagtggtattgaaggaaacttccgctcaacttgtgaaaatgttatgcaagttttgcgaacaaataaagatagtgttatggccatgatggaggcctttgtacatgatcctctcataaattggcgtctttttaacttcaatgaagttccacaaatgtctatgcttaccagcaatcatgtacctcctgttgtaaattctgaggaatctgccccaaatagagagcttccccaccctcagcgtggtgcccgtgaaagagaacttctacaggctgttaatcaacttggtgatgctaatgaggttctgaatgaacgtgctgtggttgtcatggctcgtatgagtaataagcttacaggacgggatttttcaacttgttcttcagtatcaaacaactcccttcagcatgctgtggaccatagcagcttaatttctggagacactcgtgaagttgatcatgctttatctgttaagttgcaagttcaaaagttaataatccaagcctcctcacatgaaaatttgtgccaaaattacgtagggtggtgccctttctggtaaatctcaaaggagcagtaaattttgtatatagtctgtacatagtgaaaacctgccatggatccaagtccattttgcccccctcttccccctttcacctattagcatctttaatttgtgtatatgtacagtaatttgtctcctgacactgcaatgaaaaaaatgaagcatagcacatacttggtccttgtagttcagaccacattttaactgggtctttgtaaatatcacctttccattagggagtgtgtgttgtaatatatgttcaattaatatccctacaagatggaaatggaactagatcatagttgtgagaaggcattttttgacctgatgtttgatatctcagatgagacaatagttattgtggattaaaatacgaagcagaaaaactacacataaatgatgattacactggtgctcggtttggtcatg

SEQ ID NO: 28 - DNA, 2076 bp, Glycine max
  source 1..2076: mol_type = genomic DNA
atggctggcagcaaccaagtcaatctcaatgaatctaggagtgttgttcctttaaatacatgggtgcttatctccaatttcaagttgtcttacaagcttctcagacgtgacgatggaacgttcaatcgtgagttggcggagtatctcgataggaaggtccccgcaaacgcaattcccgttgaaggtgtgttttcaatcgatcacgttgatagaaacgcgggtttattctaccgtgtgtacttaccaacctcaggaaacgaggctcaatggggtatcagagaccttgagaagccgttaagtactacagaaattgttcctgtaatcgtattctttcacgggggaagcttctcgcattcctcggccaacagtcacatctacgacaccttctgccgccgccttgtgaggatttgcaaggctgctgtggtgtctgtgaactaccggcggtcaccggagcatcggtatccttgtgcatacgacgacggctgggcggcattgcggtgggtgaagtcgagagcatggctgcaaagtgggagggaggccaaggttcatgtctacttggcaggggacagttctggagggaacattgttcatcatgtggcagtgagggctgcagaggaggaaattgaggtcctggggaatattcttctccaccctttgtttggtggggagaagagaacggaatcagaattgagattggatgggaaatactttgtgaggcttaaggatcgtgattggtattggagagcttttctacctgaaggagagaatagagaccaccctgcttgcaacccttttggaccaagggggagaagcattgaaggactcaaattccctaaaagtcttgtttgtgtggctggtttggatcttctgcaggattggcaattggcatatgcaaaaggtcttgaggattgtggacaacaggtcaagcttctttttctcaaggaagccactattggcttctacttcttgccaaacaatgaccatttctattgcctcatgaaagaaatcaacaactttgtcaattctgattctgactgttaaatggctgggagtaacgagcttaaccccaacgattctaagatggtggttccgctgaatatgtgggttctgatctcaaatttcaagttggcatacaatcttcttcgtcgcccggacggtactttcaaccgggacttagcagagttccttgatcggaaggttcccgcaaatgccaaccctgtggacagagtgttctcctttgatgttgttgttgaccgtgaaacgaatcttctgactcgaatctatcgtcctactgagggagaagaacgttcggtgaacattcttgaccttgagaagcctgtgagctctgaggttgttcctgtcatcatatttttccatggtggaagttttgcgcattcttcagccaatagtgctatatatgacaccctttgtcgtcgcctagtgggtatctgtaaggctgttgtggtgtctgtgaactataggcgtgcacctgagaataggtatccctgtgcatatgatgatgggtggacagctcttaagtgggttagttctagatcttggctccagagtaagaaggataagaaagttcatatctacttggctggggatagctcaggtgggaacattgtgcaccatgttgccctcaaggccgtggagtcgggaattgaagtgtttgggaatattctgctcaacccattgtttggtgggcaagaaagaaccgagtctgagaagcgcttagatgggaggtattttgttagagttaaggatcgagactggtattggagggcttttcttcctgaaggggaagatagagaccaccatgcatgcaacccgtttggccccaaggggaagagtcttgaagggatcacctttcccaagagccttgtggtggttgctggtttagaccttgttcaggactggcaattgggttatgccaaagggcttgagaaggctggccaagaagtgaaattgattttcctggagcaagcaaccattgggttttatttgctgccaaacaatgagcacttctctcctgttatggatgagataaaatattttgtcagctctgactgttga

SEQ ID NO: 29 - DNA, 1542 bp, Glycine max
  source 1..1542: mol_type = genomic DNA
atggctcaaacgttaaaacttaaaacacgtctgtctctgttttgtggtgagaagaagagagagacagacagaacaatgggggaagcaaagggaaaggaagaagaagtgttagcatcagttcactcaaccgtgttcaaagaatcagaaagccttgatggaaagtgtgttaagattgagggctatgatttcaaccgcggcgtcaattatccgttgctactcagctccatggtcacaactgggtttcaagcttccaacctcggtgatgccatccaagtcgttaatcaaatgctagactggaggctagttgatgaacccgtagctgaggattgcagtgaccaagagagggatttggagtaccgaaaatctgtgacgtgtaaagtgtttctgggtttcacttctaatcttatttcttctggtgttagagatactgtgaggtttcttcttcagcatcgcatggttgatgtagttgttacaactacaggtggcattgaagaagatcttataaagtgcctggcaccaacatataaaggagatttctctttgcctggagcatatctgcgctcaaaagggttgaatcggattggtaatttattggtcccgaatgacaactattgcaaatttgaggactggattattcctatctttgatcagatgttgggggaacaaaataatgagaatgtgttgtggacaccatctaagttaatagctcgattgggaaaagaaataaaaaatgaaagctcttacctttactgggcatacaagaacaacattccagttttctgtccgggcttaactgatggctcattgggggacatgctgtacttccattccttccgcaatcctggtctgattgtggacatagtgcaagatataagggcaatgaacggtgaagcagtacatgcaagtcccaggaagaccggaatgataattttaggaggtggccttccaaaacatcacatttgcaatgccaatatgatgcgcaatggtgcagactatgctgtttttatcaatactgcacaagaatatgatgggagtgattctggagctcgtccagatgaagctgtttcatggggaaagatacgagactctgctcagactgttaaggtacactgtgatgcaacaatagcatttcctctgctggttgctgaaacgtttgcttcaagagtgaaacctcaccattgatgaaggttaaagatgaatttactctgaagttcattttaattgtgtgtgtttgaggatcttgatggacaagttcggcattggtgttcaatttgactatcatttgattcctaagctcctcttagcagtggagacctttttgaccattcaaaggtaatccattaatcctgtcagtgaggaagtcaggaattatagatgtgaggatcattgtactaatatctttagggaaaataaggctaagaaaactaactgatcatatatggcatttttaatattttccaactgatggctgcatttcatgttggtaaaaagttttaattactaattcctttctgtgaattatagcagtttgttaccgttgca

SEQ ID NO: 30 - DNA, 1763 bp, Glycine max
  source 1..1763: mol_type = genomic DNA
catcatctctatatatactcactacgcacaatcatgtttctcacctatcatctctatctaatttccatgcggtactatgttttctttgcaagtactagcacaaacagctagctactatttttgaacttgtcataattagtctctaattctaattagccatacattgaacacaccagcacaccttaaacgtaagtggtatttgttccacacaggtacactatgccttcactctcagaagcctaccgagcccaccccgtgcacgttcaacacaagcaccctgacttaaactccctacaagaactccccgagtcttacacttggacacaccatagccatgatgatcatactcctgcagcttccaacgagagtgtccccgttattgatctcaacgacccaaatgcttcaaagttgatacaccatgcatgcataacttggggagcgtaccaagtggtgaaccatgccatacccatgagcctcctccaagacattcaatgggttggggagacactcttctctctcccttgtcaccaaaaacaaaaagcagctcgttcccctgacggtgctgatggctatggccttgctcgcatctcttccttcttccccaaactcatgtggtctgagggattcacaattgttggatcccctcttgagcattttcgtcaactctggccccaagattaccacaaatactgtgatatcgtcaagcgatatgatgaagccatgaaaaagctagtgggaaagctgatgtggctgatgttggattctctgggtattacaaaggaagacctgaaatgggccgggtccaaaggccaattcaaaaagacatgcgcagccttgcaattgaactcttacccgacttgtccggatccggatcgggccatgggtctggccgcccacaccgactccacccttctcacaatcctttaccaaaacaacataagcgggttgcaggttcaccgaaaaggcggcgggtgggtgacggtggcaccagtccccgaagggcttgtgatcaatgtaggcgacctcctccacatattgtcgaacgggttgtacccgagtgtgctccaccgggtcttagtgaaccggatccagcaaaggctttcagttgcgtatttatgtgggccccctccgaatgtggagatatgtccacatgcgaagctagtgggcccaaataagcctcccctttataaggcagtgacttggaatgagtaccttggcactaaagcaaagcatttcaacaaggcactctcaaccgttcggctttgtgcacctagctagctattagcggttcgtttgatgtaaaccagcctcacacaattgcttcgatccttccctcttgcatggtctattttctaccaaacatgaatgaaacacctcttctttttcttttttttgcttaatatgaatggaaacttggtggggatcgatgccaacaataaataagttcaagagggtgtatatgtatgtagatatatttgtttggctctaaactggagagagaatactggggataagatagtctcatccgtgtttcatttgtctttgtctttgtctggaaagggacatcactaaattgtcgaagctgattattcaattcgaccacaggactcgccaaaaaacattttctttccaactcggtttctttatttatttatgttcatattatttttagggtgtaagcatttatgatatatacttttaacttgtacctttataatgagaaggagtttggtccaataacagatcgcttaattataaagatc

SEQ ID NO: 31 - DNA, 2370 bp, Glycine max
  source 1..2370: mol_type = genomic DNA
ggaagcggtgtaaaagtaacagaggcggatagaaccgaaaaacccaacccgaccccgtgagcgtgcgtgactgtggcagtttctaatccgttctctctctctctctgctacctcttacctagctcgctacctcacgtaatcgttcaccgttcacctttttcttctccaatcgcaaccaaacttctctctctccctctctccctctctctctctcatgttcttgtagcggaaccgttcttgcttctcggagaacatggcctccatctcctccgccgaactcaattacctcgtttatcgctaccttcaagaatcaggtttcacgcatgctgcttttacttttggttatgaagctggtattaacaaatgctccattgatggaaatttggtgccgtcaggtgctcttgttacatttattcagaagggtctacagtactttgagatggaagccaacttgagtaattgtgatgcagacctggaagaagatttttcattcttacaacctttggatcttatcacgaaagatgtgcatgaacttaggcaaatgataagtgaaagaagaaaaaaactacaaaaggaaagaaagaaggaatttgaaaaggagcatgaaggtgaacgtgtacgggtaagagaaaaggataggcgtgaaagggagaaagaagttgaaaaggatagacgtgaaagggataaagaagttgaaaaggatagacgagaaagggagaaagaagttgaaaaggatagacgagaaagggagaaagaagttgaaaaggatagacgtgaaaaggagaaagaagttgaaaaggaaagacgtgaaagacagaaagaagttgtgaaggatagggagaaggtagaaaatgataaagaacgagaacaacaacacggagaccagactgtcagaaaaatggtcatagatcatgaagatagggttcctgttgtaaagcttgaagagaatggagcagttggagggccagaatcaatggacatctctactacatcaacatctcagctatttgaaattcctagttcagatgtgacaattttggaaggtcatacttcagaggtgtgtgcttgtgcatggagccctacaggatctcttcttgcatcaggatctggagattcgacagctcgtatttggacaatagcagaagggagatgtaagcctggttcacagaacagccctttgaatgtgttggtgttgaagcatgttaggggtaaaacaaatgaaaagagtaaagatgtcactacacttgactggaatggggaagggacacttctcgcaactggttcatatgatgggcaagcaagaatttggaccaccaatggtgaactgaagagtacattaagcaaacacaagggaccaatattctctctgaagtggaataagaaaggagattatcttctgactggaagttgtgatcaaactgcaattgtgtgggatgtgaaggcagaagaatggaaacaacaatttgaatttcattcaggtccaacacttgatgttgattggcgcaacaatgtgtcttttgccacaagttccacagacaatatgatatatgtttgcaaaattggagaaacccgccctataaaaacttttgctgggcaccagggtgaggttaattgtgtcaaatgggatccctcaggttcattgctagcctcctgttctgatgatatcacagcaaagatatggagtatgaagcaggatacttatcttcatgacttaagggagcattccaaggagatatataccatcagatggagccccactggtcctggtacaaataaccctaatcataaactggtgttggctagtgcttcatttgactcaactgtaaagctatgggatgtggaacttgggaaactcatgtacagcttggatggacacaggcatcccgtatattctgttgctttcagtcccaacggcgattatctagttagtgggtctcttgacagatccatgcacatatggtcattgagggacggcaagattgtcaaaacatacacaggcaatgggggcatttttgaggtctgctggaataaggagggtgacaagattgctgcatgttttgctaataatacagtttgcgttttggattttagaatgtaatatctgtgtagcaccaatttctccacactagtacattaaattctttcttttcaccccttgagatgagatcggtggaagtgtattttgggtgggtggtaggttacccattttaaccatagttgtaggtataactcattgttcgtggatcccgttgtaatttgctataacattagtaaaaagtagtttgtaggagtgcattcttaataagccgaagtgtatagatttagttagataatatcctgtacaattatcgcttgacttgtgttttttgt

SEQ ID NO: 32 - DNA, 847 bp, Glycine max
  source 1..847: mol_type = genomic DNA
agaaaaaataatagagaaaaggaattatataaatccatttcctccttcctagtctctgtcttctccgcccccactcacccaccctccatacacactctctctgagtcggtcgagttgaatcggtgagtcagtgaccgagtcgaaccatgtcggacgaagaacaccatttcgaatccaaggccgatgctggagcctccaagacctatcctcaacaagccggaaccatccgcaaaaacggttacatcgtcatcaagggtcgtccctgcaaggttgttgaagtttctacttccaagactggtaagcacggtcatgccaagtgtcactttgttgggattgatattttcactgccaagaagcttgaagatattgtgccctcttctcacaactgtgatgttccccatgtgaatcgtactgattaccagttgattgacattgctgaggatggatttcttagcttgcttactgaaaatgggaacaccaaggatgacctgaagcttcctactgatgagagtttgcttactcagataaaggatgggtttgctgagggtaaggaccttgttgtgtcagttatgtctgcaatgggtgaggagcagatttgtgccctgaaggatattgggccaaagaactagcctcaattgcttgtagtttgttgttttcatttaagcaaagttatatatttttgtatgatcttgttaagcctcggagtcatggaagttatcaactgtgtttatggatgtgacagaaactgttttaacatttctgtttcgatgttgccttgatattttttttgataaaatacgactatcagattatttttgttttaacgtattttgcagtttagcatggg

SEQ ID NO: 33 - DNA, 1166 bp, Glycine max
  source 1..1166: mol_type = genomic DNA
aaagtgtttggtgtgagaagagagataatggcgcagagagctcaaccagcgttggccaccacggatgtttttcagaagggagatattcattggaagcaattcaatgttgaaacatccactgcttcttcctcacctccaaaaccattgcttatttttacaccaaccgtgcctggcttataccctgtaatattgttttgccatggcttttgtattcgcactagctactactctaagctcctagcccacatagtttcacatggattcatacttgttgctcctcagctgttttccattggggtgcctatgtttggaccagaagaagttaagtgtgaaggaagagttgtggattggctagataacgggcttcaaccattgcttcccgagagcgttgaagccaaactggagaaactggttctagtaggtcacagcaagggtggaaaaacagcatttgctgtggcacttggttactgtaaaacaaagctcaagttttcagcactcataggcatagatcctgtggctggcgtatcaaagtgtaagccttgtcgatcacttcctgatatcctcacaggtgtgccacggtcctttaatctgaacatacctgttgctgtaattggaactgggttgggcccagagaaggctaattctctttttccaccatgtgctccaaatggggtgaaccataaagagtttttctctgagtgcaaaccacctagtgcatattttgttgcaacggattatggtcacatggacatgttggatgatgaaacaccaggggtaattgggacaatgatgtcaaagtgtatgtgcaagaatgggaagaagggtcctagggacttgatgagaaggactgtgggagggttggttgtggccttcttgagagcacagttgaatgagcagtggaaggattttgatgctattttagcgagtcctaatctagctcccgccaaacttgatgatgtgcgatacttaccaacatgaagtcatcacacaaacattcatttatgcagtttttattctatttctatgaaaggcctataatgtctttcaaatttgtctatataatgatgtgattcacattgcacttttcaattgggtttttacatgggacaatgcttttttttattttttattattattatagttgaaattaacaaaggattacacgt

SEQ ID NO: 34 - DNA, 3000 bp, Glycine max
  source 1..3000: mol_type = genomic DNA
atggaagctagatttggagctgaggcttatcatttttgcggtgtgggtgcttcctcggatttgaggggtgtggggaagaggtcctcagagtgggatttgaatgattggagatgggatggtgatctgttcatcgctagccggctgaatccggtggtgccggcggacggtgttggagtgggacggcagtttttccccaccgggtccgggactgggatcctggtggccggaggtccatccaacagttcttccacctctgaggaagtagatccaagggatcccaaggggaacaaggaaggggacaagaaaaggagagttattgtgctggaggatgatggtttgaatgaggaagggggtactcttagtttaaagcttggtggccatgcttctgcagtggtggatagggaagttgcaagctgggacggtacgaatgggaagaaaagtagagtgtcgggaagtacttcaaatcgagctgtttgtcaggtggaggactgcagtgctgatctgagcaaagctaaggattatcacaggcgacataaagtttgtgagatgcactccaaggccagtagggcgcttgtgggaaatgcaatgcagagattctgtcaacaatgcagtaggtttcacctgcttcaagagtttgatgaaggaaagagaagctgccgaagacgtttggcagggcataataaacgaagaaggaaaacaaatcatgaagctgttcctaatggaagttcattaaatgatgatcagacaagtagctatctgttgataagtttattgaagatactttcaaatatgcattctgacaggtcagatcagactacagatcaggatctactaactcatattctgaggagtcttgctagtcagaatggtgagcaagggagcaagaacatagctaaccttttgcgagaaccagagaatttgttgagagaagatggttcatcaaggaagtcagaaatgatgtctactttattctccaatggttctcaaggctctccaactgatacaagacaacatgaaactgtatctattgctaaaatgcagcaacaagtgatgcatgcacatgatgctagggctgctgatcagcaaatcacatcttctataaagcccagcatgtcaaacagtcctccagcctactcagaagccagagacagtaccgctggacagatcaagatgaataattttgatttgaatgacatatatattgactcagatgatggtatggaagatttagaaagattgcctgtctctacaaatcttgtgactagctctcttgattacccatgggcacaacaagattcccatcagtcaagtccacctcaaacaagtggaaattctgattctgcatctgcccagtctccttctagtttcagtggagaagctcagagtcgcacagatcgaattgtttttaagctcttcggtaaagagccaaatgattttcctcttgtacttagagcacagattcttgactggttatctcacagtcctactgatatggagagctacatacggcctggttgtattgttttgaccatttatctgcgtcaggctgaggctttgtgggaggaactttgctatgatctgacttccagtttgaataggctgttggatgtctcagatgatactttttggagaaacggatgggttcacatcagggtgcagcatcagatggccttcatttttaatggtcaagttgtcatagatacctccttacctttcagaagtaataattatagcaagattttgactgttagtccaattgctgcaccagcatcaaagagagctcaattttctgttaagggtgtcaacctgatacgccctgccactaggttaatgtgtgctttagaagggaaatatctggtctgtgaagatgctcatatgtcaatggatcaaagctccaaagagcctgatgaacttcagtgtgtccagttttcatgctctgtccctgtgatgaatggaagaggatttattgaggaggatgtttgctcagaaatctgtacacttgagcctcttctagaattaagtgaaactgatccagatattgaagggactggaaaaattaaagccaaaaatcaagccatggattttattcatgaaatgggttggcttcttcacagaagccagttgaaattaaggatggttcaattgaactctagtgaggatctctttccattgaaacggttcaagtggcttattgagttttccatggatcatgattggtgtgcagcagtgagaaaactgttgaacctactgcttgatggaactgtaaatacaggagatcatccttccttgtatctcgcgctttcagagatgggtctccttcacaaagctgtaaggagaaacagcaagcagttagtggagtgtcttttgagatatgttcctgagaatatttctgataaactaggacctgaagacaaggcactggttgatggcgagaatcagaccttcttgtttagacctgatgttgttggtcctgctggtttgacaccactccatatagcagccgggaaagatggttctgaggatgtacttgatgctttgactaatgatccttgcatggtgggaattgaagcatggaaaaatgctcgtgacagcacaggctcgacacctgaggattatgcccgtttacgtggccattatgcttacattcacttagtgcagaaaaaaattaataagagacaaggagcagctcatgtggtggttgagattccaagcaatacgacggagagcaatacaaacgagaagcaaaatgagttatccactacctttgagattggaaaggctgaagtaatacgtggccaagggcactgtaagctttgtgacaagagaatttcttgcagaactgcagtaggcaggtctttggtatacagacctgcaatgctctcaatggtggcaatagccgctgtctgtgtctgtgtggcccttctgtttaaaagctcccccgaagttatatgtatgttccgacccttcaggtgggagaatttggactttggaacaagctaa

SEQ ID NO: 35 - DNA, 1953 bp, Lactuca sativa
  source 1..1953: mol_type = genomic DNA
cattttttttccaatatttttgcgctttctggtaacacaaatttcaagtttccaccttttttccacagccgcgaacgaccgatggaaagtccaccgtctccgcctctgcctctgccactgcctctaccgccgccaattgaggccgacgtcgactctaccgcgaaccccaactctggggcctccaatcttactgtcgagaaacacactgaaccaaactccgcaacgcatgccacggttatcaagaaaaagatgaagaataaaaagaaaaggaaggacgtaccaaaatcccacaccgaagtttcttctgatccacctcaacaatcgttgaattccatttcttcttcttctacgtctagttcatttcatccaagatcgaaaggtatccgtctctcaagtaaccgcagaaaccctagagttcttagcagccccgtttcgcaaagacatgaaggaaccgaagctgacgcgcttgctctccctcttggaatgtcgatcgctgcttttgttgctcaggtgttggaaaagaaagaagcaactggagaaaagatgtctatagatcatctctcccagatatgcactttggcagttaaagaatcattgtctcatgtttttggcaacaaatttgattactttgtaagcaacttcgaaagatcatttcagagcaccttgatgacactcagagtaatcaatgaaacatcagaaaacagtaaaagatcatatcaacacgtggaagggagttcaacctatgattttcattttgatatgaaagaaaacacagccacaagccaagaacaagccaccattgtttccgaatacgaagaaaacacacatagagatcctgtttccaatgaacttgcaattcttcatgatccaagaattggtcaacagttgactttaagcactccaaatcgacatcacatgcttagcacctttgaaaggtctttatcagaacaagttcgttcaaatgatctcaaagcattcgaactcagtctcactatgcaaaagatgaaacttaaagaagcacaaatcgcagtcaattgtgattcaaatatcttagaaaggtttaagttatcaatgggcatctcaaaagccaactttagagccgagaaatttaaaaccgaattagaagattctagacacgcgcaactccttaaaaaatgcgctgattgtctcgttgctggtttgctcattatgcttgctggtcttgcttatggaacctacattcattcctatgaaaaactcattgaagccaccgaatcatgcacacccatcaagggttcgaattcatggtggattccaaagccaatggcgacatttagttcgggattccaaaccctaaggtgtcaagttcaagttatagtccgttggctatttgggcttttaatgattggtgcaatcacttatgtactccttcgaagatccggttccacaaatcaaacaatgccgattacttttatccttctcttacttggtggtgtgtgtggtgtcgccggaaaattttgcatcgacacattgggcggcagtgggtaccggtggttgatttattgggaggttctttgtttggtgcattttttctcaaatatttgtacttccatgttgtttcgtattcttcatgggcctgttgctgttactgtgactgatgggtcgattagcagtaagttattcccgtattggtttaggcgattggtgttttatgccgtggtgttggtttatttgcctttggcttgtgggatgattccttttgcggggcctgttgagtggtttcgacattttgggtcgattgtgatgggtcgggttgtgggtccggatgaatgatgtttgtaaaaattggaaaagtatgtaaaggagaatggttacttgttctatttatgttttgtgatcggttttttgaattttggttgagaaattgatgtaaatgtggtttggattcacgaagtatgagttattaaatcgagt

SEQ ID NO: 36 - DNA, 1953 bp, Lactuca sativa; identical to SEQ ID NO: 35
  source 1..1953: mol_type = genomic DNA

SEQ ID NO: 37 - DNA, 2946 bp, Nicotiana benthamiana
  source 1..2946: mol_type = genomic DNA
atgggagtaccagcattttataggtggttggctgatcgatacccgctatcaattgtggacgtggtagaggaagagcctaaagaggacagtcatgggctgtttgttccagtggatgtttcaaagccaaaccctaatggcatggaatttgataacatgtacttggacatgaatggtatcattcacccttgttttcatcccgagggaaagcctgcaccagcaacttacgatgacgtattcaaatcaatttttgtctacattgaccatcttttctcattagtccgtccaagaaaactcctctatatggctattgatggagtggctcctagagctaagatgaaccagcagcgaaccaggcgttttagagccgctaaagatgccgctgaagctgaagctgaagaaaaaaggttgagggaagagtttgagatggaggctgctagtttattgcctacaggaaagcccgagacatctgattcaaatgttattactcctggtactccctttatggcagtactagcagtagctcttcagtattatatacaatcgaggttgaataaaaatgcaggctggcggtttaccaaggttatcctttctgatgctaatgttcctggtgagggtgaacacaaaattatgtcctacatccgcttgcaacgtaaccttcctggcttcgacccaaatacccgtcattgtctttatggcctagatgcagatctgattatgctgtctcttgctactcatgaagtccatttttcaatattgagggaggtgattacatcaccaggtcagcaggagaagtgttttgcctgtggtcaggttggacatcttgcagctgattgtcgcggtacaaacggaaaccaaggcgaagatggaaagggtgtgaatgaaacacctattcacaagaagaaatatcagtttctcaacatctgggtgttgcgagaatatctgcaatatgatttggagatttacaaccctccatttcggatagactttgaaagggtggtggatgattttgtttttttgtgtttttttgttggaaacgattttcttccccatatgccaacactggagattcgagagggtgccatcaatctgctgatgtgcatctatagaagagagtttacaacaatgggtggttatcttacagatgcgggtgaggtgtttttggatagagtagaacattttattcaggcggcagcagtttatgaagaccaaatctttcaaaagagagctaggataaagcagttgatagaaaataatgaaagagcaagaaatgaggttaatgctcagccacagcctccggcggaggacaaggtgaagttaggggagcctggatacaaggaaaggtactattccgagaaatttggtgtatcaactccagagggtattgatgaagtcagacaagatgtaatgataaatgtcaacatttcaggttcagaagtatgtggaaggattatgttgggtttgccggtattactaccaaggttttacccatatcattatgctccatttgcttctgacctcaaaggtctggcggacttggaaattactttcttcccaggggaaccgtttaagccctttgatcagctaatgggtgttctgccagctgcaagtgcaaatgctcttcctgaaaagtacaggatgctaatgatggattcatcatcgccaatttctgatttttacccaacagattttgaacttgacatgaatggaaagcgttttgcatggcaggctgttgtaaaattgccgttcatcgacgagaagaagttacttgctgaaacgaaaaagcttgaagacacattatcggaagatgagcaactcaggaacagtgtactgttcgatttgctctatgttcatcatcaccatactttggctccatatatcatttcgtactatcatcacaataataaggtgcctccaggggaacaaaggccatggctaattgatgcaaatctgagtggtggaatgaatggattcatttggttatctgagcggaatggcctgagagatggggtttgttccccactcaatggattggaagatatttcaaataataaaattttaaatattacctttctgaatccagtacctcataagcatattccgaagcctccggaaggagtatatatgcccaagaagattttgagagctgttgatataaaacctttccctgtattatggcatgaagatcatggtacccggcgtcagctaggcaaagataggggtccagtacctggggcaatagcagggcctactctgggagaagcagcacatcggctgcttaacaacacattgaatatcaaacccggtggaacaaattctggagtgttggagcaatcatatcctcgaaatttcctcggcaaccatgttctgaatagaccaaggccagctggaccgtctggctatgaaggtggattttatgatcaaatgagttgcaggaatttttctcccaatcaccggccacgatttccaggaccagttggccgcgcaaatggtttttttgaggatccttcttattctagcaattttatgcctcgtggtgcatctggaaatcccagatatgctccatccccttatgagttgcaaagccctaggcaaaatttcaggatgcaagacaggcattcatatcaagatcagtatcatagcatgaggaatgaaatgtctgtgttgacaattgaaagtggttcaagaacgaggccttattcagctgtcccaaggatgctaaatccaggacaactttctaacgtccgcccaccgccattcacaccaagcgtgggtccacttccttcacccccactgcagtggattaataaacctgcaaggggaactacatcaatgcacttaaagcatcaagaaaatttaaaagggtcggggtacgagaagcaagtcaagcaggtttaccaaatcaaaagccgaccaactcagagtccccaagtaacaggaacacaacat

SEQ ID NO: 38 - DNA, 2913 bp, Nicotiana benthamiana
  source 1..2913: mol_type = genomic DNA
atgggagtaccagcattttataggtggttggctgatcgatacccgctatcgattgtggacgtggtagaggaagagcctaaagaggacagtcatgggctgtttgttccagtggatgtttcaaagccaaaccctaatggcatggaatttgataacatgtacttggacatgaatggtatcattcacccttgttttcatcccgagggaaagcctgcaccagcaacttacgatgacgtattcaaatcaatttttgtctacattgaccatcttttctcattagtccgtccaagaaaactcctctatatggctattgatggagtggctcctagagctaagatgaaccagcagcgaaccaggcgttttagagccgctaaagatgccgctgaagctgaagctgaagaaaaaaggttgagggaagagtttgagatggaggctgctagtttattgcctacaggaaagcctgagacatctgattcaaatgttattactcctggtactccatttatggcagtactagcagtagctcttcagtattatatacaatcgaggttgaataaaaatgcaggctggcggtttaccaaggttatcctttctgatgctaatgttcctggtgagggtgaacacaaaattatgtcctacatccgcttgcagcgtaaccttcctggcttcgacccaaatacccgtcattgtctttatggcctagatgcagatctgattatgctgtctcttgctactcatgaagtccatttttcaatattgagggaggtgattacatcaccaggtcagcaggagaagtgttttgcctgtggtcaggtgggacatcttgcagctgactgtcgcggtacaaacggaaaccaaggcgaagatggaaagggtgtgaatgaaacacctattcacaagaagaaatatcagtttctcaacatctgggtgttgcgagagtatctgcaatatgatttggagatttacaaccctccatttcggatagactttgaaagggtggtggatgattttgtttttttgtgtttttttgttggaaacgattttcttccccatatgccaacactggagattcgagagggtgccatcaatctgctgatgtgcatctatagaagagagtttacaacaatgggtggttatcttacagatgcgggtgaggtttttttggatagagtagaacattttattcaggcggcagcagtttatgaagaccaaatctttcaaaagagagctaggataaagcagtcaatagaaaataatgaaagagcaagaaatgaggttaatgctcagccacagcctccggcagaggacaaggtgaagttaggggagcctggatacaaggaaaggtactattccgagaaatttggtgtatcaactccagagggtattgatgaagtcagacaagatgtagtccagaagtatgtggaaggattatgttgggtttgccggttttacccatatcattatgctccttttgcttctgacctcaaaggtctggcggacttggaaattactttcttcccaggtgaaccatttaagccctttgatcagctaatgggtgttctgccagctgcaagtgcaaatgctcttcctgaaaagtacaggatgctaatgatggatccatcatcgccaatttctgatttttacccaacagattttgaacttgacatgaatggaaagcgttttgcatggcaggctgttgtaaaattgccgttcatcgacgagaagaagttacttgttgaaactaaaaagcttgaaaacacgttatcggaagaggagcaactcaggaacagtgtaatgttcgatttgctctatgttcatcatcaccatactttggctccatatatcatttcgtactatcatcacaataataagttgcccccgctggaacaaaggccatggctaattgatgcaaatatgagtggtggaatgaatggattcatttggttatctcagcggaatggcctgagagatagggtttgttccccactcaatggattggaagatatttcaaataataagattttaaatattacctttctaaatccagtacctcataagcatattccgaagcctccagaaggagtatatatgcccaaaaagattttgagagctgttgatataaaacctttccctgtattatggcatgaagatcatggtacccggcgtcagctaggcaaagataggggtccagtacctcgggcaatagcagggccttctctgggagaagcagcacatcggttgcttaagaacacattgaatatcaaacccgatggaacaaattctggagtgttggagcaatcatatcctcgaaatttcctcggcaaccatgttctgaatagaccaaggccagctggaccgtctggctatgaaggtggattttatgatcaaatgagttgcaggaatttttctcccaatcaccggccacgatttccaggaccagttggccgcgcaaatggttcttttgaggatccttcttatttatctagcaattttatgcctcgtggtgcatctggaaatcccagatatgctccatccccttatgagttgcaaagccctaggcaaaatttcaggatgcaagacaggcattcatatcaagatcagtatcatagcttgaggaatgaaatgtctgtgttgacaattgaaagtggttcaagaacgagaccttattcagctgtgccaaggattccaaattcaggacaactttctaacatccgcccaccgccattcacaccaagtgtgggtccacttccttcacctccgccacagtggattaataaacctgcaaggggaactacattaatgcacttaaagcatcaagaaactttaaaagggccggggtacgagaagcaagtcaagcaggtttaccaaatcaaaaaccgaccaactcagagtcccgaagaaacaggaacacatcaa

SEQ ID NO: 39 - DNA, 5859 bp, Nicotiana benthamiana
  source 1..5859: mol_type = genomic DNA
atgggagtaccagcattttataggtggttggctgatcgatacccgctatcaattgtggacgtggtagaggaagagcctaaagaggacagtcatgggctgtttgttccagtggatgtttcaaagccaaaccctaatggcatggaatttgataacatgtacttggacatgaatggtatcattcacccttgttttcatcccgagggaaagcctgcaccagcaacttacgatgacgtattcaaatcaatttttgtctacattgaccatcttttctcattagtccgtccaagaaaactcctctatatggctattgatggagtggctcctagagctaagatgaaccagcagcgaaccaggcgttttagagccgctaaagatgccgctgaagctgaagctgaagaaaaaaggttgagggaagagtttgagatggaggctgctagtttattgcctacaggaaagcccgagacatctgattcaaatgttattactcctggtactccctttatggcagtactagcagtagctcttcagtattatatacaatcgaggttgaataaaaatgcaggctggcggtttaccaaggttatcctttctgatgctaatgttcctggtgagggtgaacacaaaattatgtcctacatccgcttgcaacgtaaccttcctggcttcgacccaaatacccgtcattgtctttatggcctagatgcagatctgattatgctgtctcttgctactcatgaagtccatttttcaatattgagggaggtgattacatcaccaggtcagcaggagaagtgttttgcctgtggtcaggttggacatcttgcagctgattgtcgcggtacaaacggaaaccaaggcgaagatggaaagggtgtgaatgaaacacctattcacaagaagaaatatcagtttctcaacatctgggtgttgcgagaatatctgcaatatgatttggagatttacaaccctccatttcggatagactttgaaagggtggtggatgattttgtttttttgtgtttttttgttggaaacgattttcttccccatatgccaacactggagattcgagagggtgccatcaatctgctgatgtgcatctatagaagagagtttacaacaatgggtggttatcttacagatgcgggtgaggtgtttttggatagagtagaacattttattcaggcggcagcagtttatgaagaccaaatctttcaaaagagagctaggataaagcagttgatagaaaataatgaaagagcaagaaatgaggttaatgctcagccacagcctccggcggaggacaaggtgaagttaggggagcctggatacaaggaaaggtactattccgagaaatttggtgtatcaactccagagggtattgatgaagtcagacaagatgtaatgataaatgtcaacatttcaggttcagaagtatgtggaaggattatgttgggtttgccggtattactaccaaggttttacccatatcattatgctccatttgcttctgacctcaaaggtctggcggacttggaaattactttcttcccaggggaaccgtttaagccctttgatcagctaatgggtgttctgccagctgcaagtgcaaatgctcttcctgaaaagtacaggatgctaatgatggattcatcatcgccaatttctgatttttacccaacagattttgaacttgacatgaatggaaagcgttttgcatggcaggctgttgtaaaattgccgttcatcgacgagaagaagttacttgctgaaacgaaaaagcttgaagacacattatcggaagatgagcaactcaggaacagtgtactgttcgatttgctctatgttcatcatcaccatactttggctccatatatcatttcgtactatcatcacaataataaggtgcctccaggggaacaaaggccatggctaattgatgcaaatctgagtggtggaatgaatggattcatttggttatctgagcggaatggcctgagagatggggtttgttccccactcaatggattggaagatatttcaaataataaaattttaaatattacctttctgaatccagtacctcataagcatattccgaagcctccggaaggagtatatatgcccaagaagattttgagagctgttgatataaaacctttccctgtattatggcatgaagatcatggtacccggcgtcagctaggcaaagataggggtccagtacctggggcaatagcagggcctactctgggagaagcagcacatcggctgcttaacaacacattgaatatcaaacccggtggaacaaattctggagtgttggagcaatcatatcctcgaaatttcctcggcaaccatgttctgaatagaccaaggccagctggaccgtctggctatgaaggtggattttatgatcaaatgagttgcaggaatttttctcccaatcaccggccacgatttccaggaccagttggccgcgcaaatggtttttttgaggatccttcttattctagcaattttatgcctcgtggtgcatctggaaatcccagatatgctccatccccttatgagttgcaaagccctaggcaaaatttcaggatgcaagacaggcattcatatcaagatcagtatcatagcatgaggaatgaaatgtctgtgttgacaattgaaagtggttcaagaacgaggccttattcagctgtcccaaggatgctaaatccaggacaactttctaacgtccgcccaccgccattcacaccaagcgtgggtccacttccttcacccccactgcagtggattaataaacctgcaaggggaactacatcaatgcacttaaagcatcaagaaaatttaaaagggtcggggtacgagaagcaagtcaagcaggtttaccaaatcaaaagccgaccaactcagagtccccaagtaacaggaacacaacatatgggagtaccagcattttataggtggttggctgatcgatacccgctatcgattgtggacgtggtagaggaagagcctaaagaggacagtcatgggctgtttgttccagtggatgtttcaaagccaaaccctaatggcatggaatttgataacatgtacttggacatgaatggtatcattcacccttgttttcatcccgagggaaagcctgcaccagcaacttacgatgacgtattcaaatcaatttttgtctacattgaccatcttttctcattagtccgtccaagaaaactcctctatatggctattgatggagtggctcctagagctaagatgaaccagcagcgaaccaggcgttttagagccgctaaagatgccgctgaagctgaagctgaagaaaaaaggttgagggaagagtttgagatggaggctgctagtttattgcctacaggaaagcctgagacatctgattcaaatgttattactcctggtactccatttatggcagtactagcagtagctcttcagtattatatacaatcgaggttgaataaaaatgcaggctggcggtttaccaaggttatcctttctgatgctaatgttcctggtgagggtgaacacaaaattatgtcctacatccgcttgcagcgtaaccttcctggcttcgacccaaatacccgtcattgtctttatggcctagatgcagatctgattatgctgtctcttgctactcatgaagtccatttttcaatattgagggaggtgattacatcaccaggtcagcaggagaagtgttttgcctgtggtcaggtgggacatcttgcagctgactgtcgcggtacaaacggaaaccaaggcgaagatggaaagggtgtgaatgaaacacctattcacaagaagaaatatcagtttctcaacatctgggtgttgcgagagtatctgcaatatgatttggagatttacaaccctccatttcggatagactttgaaagggtggtggatgattttgtttttttgtgtttttttgttggaaacgattttcttccccatatgccaacactggagattcgagagggtgccatcaatctgctgatgtgcatctatagaagagagtttacaacaatgggtggttatcttacagatgcgggtgaggtttttttggatagagtagaacattttattcaggcggcagcagtttatgaagaccaaatctttcaaaagagagctaggataaagcagtcaatagaaaataatgaaagagcaagaaatgaggttaatgctcagccacagcctccggcagaggacaaggtgaagttaggggagcctggatacaaggaaaggtactattccgagaaatttggtgtatcaactccagagggtattgatgaagtcagacaagatgtagtccagaagtatgtggaaggattatgttgggtttgccggttttacccatatcattatgctccttttgcttctgacctcaaaggtctggcggacttggaaattactttcttcccaggtgaaccatttaagccctttgatcagctaatgggtgttctgccagctgcaagtgcaaatgctcttcctgaaaagtacaggatgctaatgatggatccatcatcgccaatttctgatttttacccaacagattttgaacttgacatgaatggaaagcgttttgcatggcaggctgttgtaaaattgccgttcatcgacgagaagaagttacttgttgaaactaaaaagcttgaaaacacgttatcggaagaggagcaactcaggaacagtgtaatgttcgatttgctctatgttcatcatcaccatactttggctccatatatcatttcgtactatcatcacaataataagttgcccccgctggaacaaaggccatggctaattgatgcaaatatgagtggtggaatgaatggattcatttggttatctcagcggaatggcctgagagatagggtttgttccccactcaatggattggaagatatttcaaataataagattttaaatattacctttctaaatccagtacctcataagcatattccgaagcctccagaaggagtatatatgcccaaaaagattttgagagctgttgatataaaacctttccctgtattatggcatgaagatcatggtacccggcgtcagctaggcaaagataggggtccagtacctcgggcaatagcagggccttctctgggagaagcagcacatcggttgcttaagaacacattgaatatcaaacccgatggaacaaattctggagtgttggagcaatcatatcctcgaaatttcctcggcaaccatgttctgaatagaccaaggccagctggaccgtctggctatgaaggtggattttatgatcaaatgagttgcaggaatttttctcccaatcaccggccacgatttccaggaccagttggccgcgcaaatggttcttttgaggatccttcttatttatctagcaattttatgcctcgtggtgcatctggaaatcccagatatgctccatccccttatgagttgcaaagccctaggcaaaatttcaggatgcaagacaggcattcatatcaagatcagtatcatagcttgaggaatgaaatgtctgtgttgacaattgaaagtggttcaagaacgagaccttattcagctgtgccaaggattccaaattcaggacaactttctaacatccgcccaccgccattcacaccaagtgtgggtccacttccttcacctccgccacagtggattaataaacctgcaaggggaactacattaatgcacttaaagcatcaagaaactttaaaagggccggggtacgagaagcaagtcaagcaggtttaccaaatcaaaaaccgaccaactcagagtcccgaagaaacaggaacacatcaa

SEQ ID NO: 40 - DNA, 2937 bp, Solanum lycopersicum
  source 1..2937: mol_type = genomic DNA
atgggagttccagcattttataggtggttggctgatcgatacccactctcaattgtggacatggtagaggaagagcctaaagatgatgttccggtggatatttcaaagccaaaccctaatggcatggaatttgataacttgtacttggacatgaatggcatcattcatccttgttttcatcctgaaggaaagcctgcaccagcaacctacaatgacgtattcaactcaatttttgactacattgaccatcttttctcattagtgcgtccaagaaaactcctctatatggctattgatggagtggctcctagagctaagatgaatcagcagagaaccaggcgtttcagagcttctaaagatgctgctgaatctgaagctgaagaaaaaaggttaagagaagagtttgagatggaggctgctattttagtgcctacagaaaagcctgagacatctgattcaaatgttattactcctggtaccccatttatggcagtactatcaatagctcttcaatattatatacactcgaggttgaataaaaatgctggctggcggtttaccaaggttatcctttctgatgctaatgtgcctggtgagggtgaacacaaaattatgtcctacattcgcttgcaacgtaacattcctggcttcaacccgaatacccgccattgtctctatggtctagatgcagatcttattatgttgtctctggctactcatgaagttcatttttcaatattgagggaggtgattactccaccaggtcagcaggaaaagtgttttgcctgtggtcaggttggacatcttgcagctagttgtcatggtacaaatggaaaccatggcaaagatgggaaggctgtgaatgacacacctattcacaagaagaaatatcagtttctccatatctgggtgctgcgagagtatttgcaatatgatttagagatttacaaccctccatttcagatagactttgaaagggtggtggatgattttgtttttttgtgtttttttgttggaaacgattttcttccccatatgccaacactggagattcgagagggtgccatcaatctgctgatgtccatctatagaagggactttacaactatgggtggttatcttacggatgcgggtgaggtttttttggatagagtagaacattttattcaggcggcagcagtttatgaagatcaaatcttccaaaagagagcccggatacaacagtcgatagaaaataatgaaagggtgagaaaagaggctaatgctcagccacatcctccggcagaggacaaggtgaagctaggagagcctggatacaaggaaaggtattattctgagaaatttggtgtatcaactgcagaggatatccatgaagttagacaagatgtagttcagaagtatatggaaggattatgttgggtttgccggtattactaccaaggtgtttgctcgtggcaatggttttacccatatcattatgctccatttgcttctgatctcaaaggtctagcagatttggaaattacttttttcccaggggaaccatttaagcccttcgatcagctaatgggtgttctaccagctgcaagtgcaaaagctcttcctgaaaaatacaggatgctgatgatggatccttcatcaccaatttctgatttttacccaacagatttcgaacttgacatgaatgggaagcgttttgcatggcaggctgttgtaaaattgccgttcatcgatgagaaaaagttacttgctgaaacaaagaagcttgaagacacgttaacggacacggagcaactaaggaacagtgtaatgttcgatttgctctatgttcatcatcaccatactttggccccatatatcatttcgtactatcatcacaatgttatgattccctccagggcccaaaggccatggataattgatgcaaatttgagcggtggaatgaacggattcatttggctatctgagaggaatggcctgagagatagggttccttccccacttaatggattggaagacatcttaaataataagattctaaatattacctttctgaatccagcacctcataagcacattacgcagcctccagaaggagtatttatgcccaaaaagatgttgagagctgttgatataaaaccttttcctgtattatggcatgaagatcatggaacccggcgccatctaggcaaagataggggttctgtacccggggcaatagcagggccttcactgggagaagcagcacatcggttgcttaagaacacattgaatatcaaacctggtggaacaaattttggagtgttggatcaatcattttcccgaaatttttcgggcaaccatgttttgaataggccaaggccagctggaccatcttgctatgaaggtggattctatgatcaaatgagtcgtaggaattcgtctccgaatcaccggccacggtttcctggaccctctggacatgcaagtggcttctttgaagacccttcttatttccctagcaaccttatgcctcgtggtgcatctgggaaccccagatatgctccatccccttatgagttccaaaacactaggcaaaatttcaggattcaagatagacattcatatcaagatcagtatcatagcatgagaaatgaaatgtcagtgttgacaatcggaagtggtgcaagaacgagacctccttcaaatgcagcaagaatgccaaattctggacaactatctaacgtctgcccaccacctccatttacacaaaatgtgggtcctcttccttcacctcctctgcagtggattaacaaacctgcaagaggagctacagcaatgcactcaaagtatcaagaaacttcaaaagggcccgcgtacgacaagcaagtcaagcaggtttaccaaatcaaaagccggccaactcaagagtccccaagtaccgaaacacaacagtaa

SEQ ID NO: 41 - DNA, 2225 bp, Solanum lycopersicum
  source 1..2225: mol_type = genomic DNA
caagattccatggatcacccgcctctatctcaacccgtcgatccgccgccggtgaccaccgccgacgccatggcttccgaaattctaccggaatctttaaattccggcgttatgaagaagaagacgaagaagaaaaagaagaataaagcatgcgtcacggacccttttgaccctcattcttctgctgcctcttattcatcatcatcttgttctgctacgtcggttccaccttctactcaaaggggtattagggtttccactggtcgtcggaaccctagagtcatcattagctccggtagacagaaaagcgacaatgttgaagcacttgcacttcctctcggcatgtctattgctgctgttcttgctcaggttttggaaagaaaagatgcagccggagaaaagatatctgtggatcacctctcagagatttgtactttggctgtccgagagtctttagccaatgtattcggtgatcagtttgagtctttcgtgaggaactttgagaaatcgtttcatagtaccttgatgactctccgattgatcagtgaatcctcaatgaacagtggagtacagcaacataattgtgcagcaaggacttctgtctcagaaagatctgtgccttttatttctaacagagtagaaaatgttacatgtgatcctgacttcagtgagttccaatcggagtcatttcagcagaatagctcagataacgaactgagcaatcaagaagagagaagtgacgggacatgttttgaatcaataggtcggcagcttacccgctatgataggcaagtaaggcaacagttggcttctgcttcttcaagcatgattttatccaatacagggattagccaatctgtgcataggactttggaaagatctctcacagaacaagctaggtcgaatgaccttaagacatttgagattggtcttacaatgaggaagttgcaacttaaagaaaggcagctggctctcagttctgatgctaaccttttggagagggtcaaactatcatttggtttctctaaaacgtccttcaaaactgaaaagttcaaaaatcaagtagaagactcaaaacatgctgagctacttaaaacctgtatagactgccttgttgctggtttattcatcatgctggcgtgtcttggatatggaacttatgttttctctcacaaaagaattaccgaagctacagcatcatgcaccccttccatggagactcgttgtccaaagaagtcaatgttagttgttgtagatgctctgctctctgtgttctttcttaaagcatggatcccagggatgacattagaacacaagtcttggtggatgccccaatcaatgtcgtcattcaattcaggactgcaactcttacggtgtcaggttcaagttctcagtcgcatgctatttggtgctttcctgatattgtctatagcctatctacttctccagcgctctgcaacttcaaatcagacaatgccagttactttcattctgttgctgttgggagttggctgtggtttcgcagggaagttttgtattgacaccttgggaggcagtggatatcgttggttaatttattgggagactttatgcttgctacatttcttttcgaatgtctgtatctcgactttgttcctgatccttaatggtcctgtcacagtgtcagaaaagagcatgagcgatcgaagatttccatactggattaggagatctatgttttataccacagtacttcttattctccccttgctatgtggtctgatgccttttgctggccccggtgaatggaaagatcacttctcttcacttgtcctggaggcttttattactccagttgaattttgagtagtagagtgcaagcttatagcattatcggatgaaatactgttttgatgaagacctgtcaaggaattttgactagtagaagacgagtatgaagtatcgagcttaaggatgattatgtttctgtttacatgtaaattatgatgtatcattaggggctagatgaagatttccgggttatggatgaaacactgttttgatctgtgcctgtagcgttgtatcattaattagtactccagggttagtctaaattatgtgcatcattacagcaaaatgaggctctgtgtttacttactttttggttaatataattgggggaagcagcttttgagtgttgtaaacaaattgcaggaggatacaaataatcttgtattgaggtagga

SEQ ID NO: 42 - DNA, 2225 bp, Solanum lycopersicum; identical to SEQ ID NO: 41
  source 1..2225: mol_type = genomic DNA

SEQ ID NO: 43 - DNA, 1776 bp, Spinacia oleracea
  source 1..1776: mol_type = genomic DNA
atgggcttctcaaatggtagcttagacaacaacaattacaatttcatgaccaactcctccttcaccgacctgcttaactccggcgccgacgactcttccaagctcaccgtacctaaccggagcttatccgatcgtatcgccgaccgaaccggctccggtatcccgaaattcaagtccttaccacctccttcaatcccattatctccacctccggtctcaccttcgtcatattttgcttttcctcctggtttaagtcctactgatctcttagactcccctgttcttctctcctcttctcatgtgttgccatctccgacaacgggggcttttgcggcaaactggaaatcgtattccaacaacaacagcagcaacaacaacaacaacaaacaagaagatgtgaacaaatattcagattttactttccaaacccaaacaaggcctaatcatgtttcatccaacatgtttcaaccttcatttcttaatgaacaggctgtaagaggaagggaaaaccaaccagtacaacaaccagcacaaccccaacaacaatcatggaatttcgatcaaaagaatgatggatcttcgaactcaattacagctatggtgaaatcagagtttcctggtgagaattacaaccaaggaagcgttgtaaagtatgagacagaccaaaaccctccctaccaaccagctcaatacataagagaacagagaagatcagaagatgggtataattggagaaaatatggacaaaaacaggtaaaagggagtgaaaatccaagaagttattacaaatgcacgtttcccaattgtccaatgaaaaagaagattgagacagcaatagatggtcagattactgagattgtttataagggtagtcataatcatcctaagcctgtgaatactcggagatcttctgcagccgcttcagctgtttctgcagcttttcaaaacggttctgttcctccatcgtcctcaaatgattaccatgatcattcattaggtccgcagagatcatcctcggggcaaatggattccggtgctaccccagagaattcgtcgatttcgatggctgatgatgaggttgacatgggttctcataggagtagatcatcagctgatggtgatgaacccgaggctaaaagatggagaggagaagttgatcagaatgaatgcatgtcagctgcaccagggagtcgagcagtgagggagccaagagttgtagttcaaacaactagtgatattgatatcttggatgatggatatagatggagaaaatatggtcaaaaagtggttaagggaaatcctaatccaaggagctactacaagtgcacgcagccaggttgcccggtgaggaaacacgtggaacgggcagctcatgatcttagggcagtgatcacaacctacgaaggaaagcacaaccatgacgtgccagctccccgaggcagcgggatgagcagggcagccatggctcctaacaatgttccagtttctttaatggctataaagccatcagctgttaactatacaagtaacgctcattcgttccagactagtaatcaaggaggaatgccctcgtctttcgacggggcaggaccattatcaccttttacattacagttgttgcaaaatccggggagtttcgggttttctggctacttgaatcaacctcaatactcagacaacaacatgttttctaatactactgctacaactacttctactattaaagccaaggaggaaccaaaagaagacatgctttttgagtcattgctttgctga

SEQ ID NO: 44 - DNA, 1704 bp, Spinacia oleracea
  source 1..1704: mol_type = genomic DNA
atgtcttcctcagttgcaagcttagaacattccctttccttcaacacaagtagcttctccttcactaacctcatgaactcctcttcctccttcaccgacttcttcgcaagcacaaccaatgatgatcaacatgttttggagggtgattacaacactaacacaaaagagaagactactactaataacaataacaataacaataacaataacaataataatgacattaatagtactaattggtccggatttgatcaactcattgaagttcctaagttcaagtctatggctccttcttctctttctgtcaccgagactactcccctcgcttcgtcttcttcgtttttgaacactcctaatgcttttagtccttccatcttcttggattcgcctctcctcaatagtttcccatctccaactgtaggagcctttggtggtgagggattcaattggactcaactaggtgctaataaggatgaaggaagaaacaacaattttactttccatgcttcttcaaacaactcaattgaagggtggaatttggatgaaccgaagaagcaaaatgagatgattaacaacaaagatcaaatcaattcaaaatccgagtttactactgttaatcaaaccatgccatatgaaatgaacacaacaaaaacagatgttggtactaatatgcaaagagggtacaatgctcaaccagttcagatgaatcgagcacagaaaagagctgaagatgggtattgttggaggaaatatgggcagaagcaagtcaagggtagcgaaaatcctcggagttattacaagtgctcacacccaacttgtcctacgaagaagaaagtcgagcgatcaccagaaggttatgttactgagattgtgtacaagggaagtcacaaccatccgaagcctcaacctggtagaagatcatctgttcagaataataacaataataacagtgggtttgatggttcagagggttcttctaatacagcccttatcatgtcacagactgataattattcttcctttactcctgagacatcctctgtttctgttgatgaggaagacgagtttgatcagacttccgccatgagtaaatcagttaaagaagatgatcatgaacctgaatccaaaagatggaaaggagagaatgaaggggaagtgatgtcaggttacggaaacagaacagtgaaagaaccaagagtaatagttcagacaaccagtgaaattgatattcttgatgatgggtatagatggagaaaatatgggcagaaagtagttaagggaaaccccaatcctaggagctattacaaatgtacgagcacaggttgcccagtaagaaagcacatagagagggcatcccatgacatgagggctgttataacaacatacgaaggaaagcacaaccacgatgttcctgcagctcgtggaagcgccagctataacaatgtcccgatgccagttaggccatcagccatagccagccaacctaaccagataattggcctaagacctatcaacaacaacaccaacaacaacaacaacaacaacatatcaaatacctcaagaccagaagctcaccaggcacccttttcattgcagatgctgcaaaataatactgcaacctttgggttttcagggtttggaaatccttctgaagccaaggaagagcctatcgacaacatgctgttagaatctttttttcgttga

SEQ ID NO: 45 - DNA, 1104 bp, Spinacia oleracea
  source 1..1104: mol_type = genomic DNA
atgggaggaggttatagatcaaggaagatggaagacgaagtagcaattgaagaggcggcatcggctggggtgaagtccatggaacatttgctacgccttttatcatcgtcatcatcaccatcttcttcttctgagtgcagagaagttactgaggttacagttggtaagtttaagaggctcatctctgttctcaaccgtactggccatgctcgttttcgccgtgctcctaatctttctgtctcctctgattcttccaattcttccgactctcctccgccctttcaaccacctccccctcactccaccaaacccttgttcacaccaccacctgcacaaacaccaacaacaccgatttgtcaaccggaaaaaccgattccgttgagggttgttcgacagtcgcacaaccacccgatgactttggatttcacaaaaccgaacccgaatacaaaaccatgtgcgtcgatttcgaattcggcgtcctttagtatatcttcatcggcgaactcatcgtcgtttttgtctactattaccggagaaggaagcgtgtctaacagaaagctattcggtggatctgtttttgttccacctcctacatcgtcgtctgggtttgctgcttctgggaaacctcccctttcaacgtcttcgtcttctcatcataataagagttgtcaccaccaccaccaagagttttccgaagacttttccggtaaaatctccgggtcaacacggtgtcactgctcatctaagcggaagaagttgagggtgaagaagacgatgagagttccggcgatcagttcaaagaccgccgatattccttctgatgattactcttggaggaaatacgggcagaaacctattaaaggatctccttacccaaggggatattacaagtgcagtacagtaagaggatgtccggcgaggaagcatgtagagagggcaacagatgatccaacgatgttgattgtaacttatgaaggagagcaccgtcactctcctatacctgcatcacacgataacatctctcatcacatggtccccaccaccgctacaccagcatggttggtccctttgcagtcgcaccctattttgactttgtctgactga

SEQ ID NO: 46 - DNA, 2124 bp, Spinacia oleracea
  source 1..2124: mol_type = genomic DNA
atgacatccataacttccgttgagttaaattatatgattttccgttatcttcaagaatccgggtttacacactccgcctttgctttaggatatgaggctggaatcaataagtgccccatagatggcaatttagttccaccgagtgctctcattacctttgtgcagaaagggctacagtacttggaaatggaagctaacttgaataatagtgatgctgatatggatgaagatttctcatttatacagcctatggatcttatcactaaggatgtgaacgaattgcgacgaatggttaaagaaaagaggagaagttctaggaaggataaggagaaagaatctgaaaaggatcgtgatagagaacgaaagcgaggaagagataataggaggttagatagggagaaagatcatgttaaggatggagaaggaaaagagaaggatgaaaaagcaatgagagagaaggacgaaaaagctgcgagagagaaggacgaaaaagctgcaagagagaaggaagaaaaacttgtgagagagaaggatgaaaaagctgtgagagagaaggacgaaaaagctgcaagagagaaggaagaaaaaactgcaagagagaaggaagagaaagctgcaagagagaaggaggaaaaggctgcaagagacagggacgaaaaatctgcgagagagaaggatggaaaagctttaaaagagaaggatgaaaaatctgggaaagagaaggaagaaaaagcagggaaagagaaggaagagaaagctggaaaagagaaagaggaaaaagctggaaaagtaaaggatgaaaaatctgggaaagagagggaagaaaaagcagtgaaagagaaggaagaaaatgttgggaaagatagggaagagaaatctatcaaagaggaggaggaaaaacccaagttacaagatgtgccttctgatatggaaacagttgttgagcgagaggagaagcctgccatcaaatatgaagagaatagagtttgtcctgatcctgagcccatggatatctctccatcatcaccttccatggagtgtgaaatttctacttcagatgtgacaatattagagggccacacatccgaggtttgcgcatgtgcatggagcccttctggttcacttcttgcatctgggtctggggattcaacagctcggatttggcctatgtctgagagaggttttggatcacacaatggatccaccaatgtcgtggtgttgaagcatgttaaggggaaaacaagtgagaaaagcaaagatgttactactcttgattggaatggggagggtacccttctcgccacaggatcttatgatggccaggcaagaatttggaacacaaatggtgaactgaagagtacactaagtagacacaaggggccaatattttctttgaaatggaacaagaagggtgattatcttcttacgggaagctgtgataaaacagccattgtgtgggatgtgaaggcaggagagccgaagcaacagtttgagtttcatacaggtccgaccttggatgttgactggcgaaataatgtttcttttgcaacaagttctacagataacatgatatatgtatgcaaagttggggacagcagtcctgttaaaacctttgcaggccatcagggtgaagttaactgtgtcaaatgggaccccacggggacactattggcctcttgctcagatgatagcactgcaaaggtatggagtatgaaggaggatggttttgttcacgatttgaaagatcattcaaaggagatttacactattaggtggagccctactggttcaggaacaaataaccccaaccagcagttggttctggccagtgcatcttttgactccactgtgaaactatgggatgtggagcttggaaaactactgcgtagcttggatggtcacaaggaagctgtatactctcttgcattcagtcctgatggtgaatatttagcaagcggatctttggatagatcgttgcacgtttggtcccttaaagaagggaagattgtaaagacatactctggaaatgggggcatatttgaagtatgttggaataaggaaggtgacaaaattgctgcatgtttcgccaacaatactgtatgtgttctggatttcagaatgtag

SEQ ID NO: 47 - DNA, 936 bp, Spinacia oleracea
  source 1..936: mol_type = genomic DNA
atggcgacggaaggaggtggtggaggggcggcggcagcggcgaggaggaagccgtcgtggagagaaagagagaataatcggaggagagagagaaggaggagagccatagctgcgaagatattttccggtctccgagctcaaggaaactataatctccctaaacattgtgataacaatgaggttctgaaggctctttgttctgaagctggctggattgttgaacctgatggtactacttaccgcaagggaatgaggccgccaccgccggagattgtggctagttttaatcattttagtgcgtgttcatctcaagtgccgagtcctctatcgtcggcatttcagagtccaattcattcctaccaacctagtccgtcttcgtctgctccgcaaagcccgactcgaaacgagatcaatgcacaatcccatccgttctcgttcctctgcaattctattccgtcgagtctgccacctcttcggatatcgaatagtgccccggtgactcctctgttatcgtctccaacgtcaaaaactgctcctaagcagccagtttttaatttcgaggccattgctaatgagtctatggcatcttttaattatcctatgtttgctgcttctgctccttccagtccttcaagacggaggaactttaccccgcctaccatacctgaatgtgatgaatctgactgctctactattgattccggacagtggattagcttccaaaggttttccagacctccaattccaaattcccccacatttaacttggttaggccaatggttcagaaaatgtccttgaatgatgcaattcaggatagagcaagagttgctgaagctgagagtatgcaggttaccccttgggagggggaaaggattcatgatgtaggcatggatgatcttgaactcactcttgggagtggtactgccaggaactaa

SEQ ID NO: 48 - DNA, 2934 bp, Spinacia oleracea
  source 1..2934: mol_type = genomic DNA
atgaagcacattttcaagaagcttcataaaagtcacgaccttaatcgcactaacaatgaaaccctaactccttctccttcttctacttcgcaacctcaatcctcaacttctacttcgtcgtcctcttcggcggcaacatcgtcatcgatggcggtggcggcaacggcaacgcaatccaccgatcaaggtaattctcagcttaccggtgtcggaaacttgccggcggtgaatttggcaaagaatgatcatcttcggcaaggtgattacttttcgtctgaggaggaatttcagatgcagcttgctttagcgattagtgcttcgaattctgagtttcgagatgatccggaaaatgatcagatccgtgcggccactttgttgagtttggggaggtctaattctattgataagggccgggataaagaggcttctggtgaggccttgtcgcggcggttttgggattacaatgtacttgattatgaggagaaggtggtcgatggcttctatgatgcattcgttatttcagtggatccagcagtgcaaggaaatatgccttctcttgtagatcttgaaacaaatgccggcgattcaggttttgaggccgtgatagttaataggacaattgatcctatacttgtggaattggagcaagttgcacattgttttgcacttgactgtcctgcatccgaagttggcattttggttcaaagacttgctgaacttgttacagaacacatgggaggaccagttaaggatgcaggtatagtattggcaaaatggatggaaatcagcacagaattgagaacttctcttcatacaagtgtttttccaattggttctgtaagggttggcttgtcacgccatcgggctttgctctttaaggtattagccgacaatgtcgggataaaatgtagacttgtgaaaggaagtcattacaccggcgttgaagatgatgctattaacataataaagatggctgacgagagggagtttttggttgatctcatggcagccccaggaacactaataccttcagatgtattaaacatgaaggattctcctgttccagtatacaaccaaaaactaaacaagatcccttccttgcagccaacaaatgccagactaaagccagtaaattcaaggccaagtgggggctctcctgttgaaaataattctcattttggtcggaattcagtcattggaaagattgaatcattgccttcgtcttcaagcaccagtaatgacatgggtgctggcccttcaagagcttccagccaatcatacccaagcaatcaattggatttgccttcaacatcagcagtgggggcatctttgtttaaaccacatcgtggagcccttgctgcaggtgatggtaccaggttgaatgttaatgtagtaccctataatcaaggtaataatgatgaatcaaagaatctttttgcggaacttaatccattccatttacgagtttctggccaagcttcacagcaaaataaacccaaatacgagattgatgagtctcagagatttaagaatgatgctgtctccagaaaaccgccttctcccatgatgtggaaaaaccaccaggctatcaattatgtccccaggaataaagattatgatattgttgaaagtcaactcccgagaaataacaatggaaacaaagattccagagcggcaccagtaacttcatctagaacatcagcttcccaaaatttacgtcaaaatggctctcagttggagagttcgactccaagtggtgtaaacatcgatgtgggcagctctgttagtgttactacaactgctgcaacgggcagtggtcagaatgatgagtcttttacagcggaagtcagaattgccacccaagtgaaagactccctgaaggaagcagcctcttctggcgacatgcttgttcagggaactgaacgcaaaaaaggtggcaccagtggtggcaagtgcactactgagggtttcattggaacaggatatatattaaatgatttcgatctaagacctgtagattctaacggaaaggtagttgaccaagtgctagatgatgtgggtgaatgtgaaattttgtgggaagaccttattattggtgaaaggattggactaggttcatacggggaggtctaccacgcagactggaatggcacggaagttgctgtcaagaaattcttggaccaggatttctctggggctgctttagcagagttcaaaagagaggtccgtataatgcgaagattgcgccatcctaacgttgttttgtttatgggtgctgttactcgcccgccgcatttatcaattgtgtctgaatttcttccgagaggaagcttgtaccggattctccatcgtcctaattgtcaaattgacgaaaagcgtaggattaaaatggcgctggatgttgcgaggggcatgaattgcttgcatacaagcacaccaacaatcgtgcaccgtgatttaaagtcgcccaacttactagttgacaagaattggaatgtgaaggtttgtgattttggattgtcaaggctgaagcataatacatttttgtcttctaaatcaactgctggaacgcctgaatggatggctcctgaagttcttcgaaatgaaccatcaaatgagaaatgtgatgtttatagctttggagtgatattgtgggagcttgcaacacttaaacttccttggagtgggatgaacccgatgcaagttgttggggcagtaggtttccagaatcggcgcctagacatctccaaggaagttgatcctttggttggtaggataatatgggaatgttggcagacggagccgaacctgcgcccttcatttgcacagctctgtgtagctctgaagtctttgcagcatcttgctctctcttcacagattgaccagcaaagttcagcaacccaatcagagatttctgtaaattctacgccttga

SEQ ID NO: 49 - DNA, 354 bp, Spinacia oleracea
  source 1..354: mol_type = genomic DNA
atggctggaagaattatcgcccaaattctagtgatgggctctgggatattagttagggcttttgctcaagcttatcgtcaagctctttctaatgcctcgaaaactggtgcagctcaggaagcaatgaacaataccgtacgcagagcaagtaaagccatcacagaacaagaagcaaggcagattcttggcgttcctgagaatgcggcctgggaagatattttgaaaaaatatgatgttttgtttgagaggaatgccaagcaagggagcttctacatccaatcgaaggtccatagagcaaaggagtgtctagaagcgatacatcaaagcaaaggtcaaggttccgccaatgggtaa

SEQ ID NO: 50 - DNA, 336 bp, Spinacia oleracea
  source 1..336: mol_type = genomic DNA
atggcagcaaggatactcgcccaacttcttgtggtgggttctggggtgttaattcgggcggttggtcaagcttatcgtcaagctattgtcaatgcctcaaaaactggtgctgcgcaagaagcaatgcaaaacaccattcgtagagcaagtcaggctcttacagaacaagaagcaagacagatacttggagtccccgagaaagccgcttgggaagaaattttaagaaaatatgatgttttatttgagaggaatgctaaacaagggagcttttacctccaatcgaaggtacatagagcaaaggaacatctggaagaagtgtatcgaaataaaggttag

SEQ ID NO: 51 - DNA, 5472 bp, Spinacia oleracea
  source 1..5472: mol_type = genomic DNA
atggcttcctccgactctcgcaagcccatcactctcactcctcccaaccagatcgccaaagatatcacagggccagacaatcctataccgctttcacctcaatggcttctccctaagccaggggagactaagactgggatgtcactaggggaaaatcgtccaagcccttacactggttacggtgatcgttcagatgtcactaagtctccgggaaatggtgaggagatgcatgataaccagaagaaaagggatgttttcaggcctacttttcttgatgcagaaactgggcgtcgtgatcgctggcgtgacgaagaaagagacactaatccatcagctcgcaaggaccgttggagggagggggaaaaagagattggtgacggtcggaagaccgacaggtgggttgataatacgcccactaagaactttggagaagcacgtcgtgctccatccgacaagtgggctgatccaagcaaggagaataattatgatcagcgtcgtgagagtaagtggaacacgcgctggggtcctgatgataaagaggcagataagtggactgatgctggaaaacacggtgacatgcctcaggataaaggccaatcacaattagctaatcacgggaaggatgagagggagggtgataaccatcgcccgtggaggcctagctctttgcaaaatcgtgttaaagcagaaacattacatcaccatcctcaaatgactagcaaagaaggtcctacatttggccacgggagaggtcgtggggataatgctactttttctgttggtcgtggacgggctaattttagtgctgggagtattagtaactattctgttcatcctcactcaggttttaattctgacagaggtgataatggtcatgaagaaccttctcctttcagctataatagaacaaagttgcttgatatttaccggctgactgagataagatcttcgaacaagatattggatggattgaagcaagtttcctctctcactcaggaagaacctttggggcctctggcactttgtgcacctacgcctgaagaatcagctattatgaagggtattgacaagggggacataataagcagcggtgcaccccagaattcgaaggaaggatctacaggacgaagtccaaatgaatatgctcaatcaagacgaacgaaacatggcagcagagatgatgtatcccttcctcctgatgaagacaatgataaagttgaccatgctaaaggtacttattcaaaaatttcagagttgtcaccccgtgagaagcagatgaatgtgtatggttcaaatgtcataatggatggcaatgaaaatcctcagatatatgctgagaaatatagagttccagctgcaagagatgatgagagttctttaaagaagggtgatgaagtctctgataacagagaccctggcattaaggggcgtgtttattctcaatctggaactccgtggcggtcaccttctttggaagagcacgcaagcactcactcacgtgattggtttaatagtccagcgggaggtggtattcgctccaaaagctctgacatcagctggtcacaagcaccaaaagacaactcatctgttttgcctgatccaaatgctcacaatgaactcaagtggcaagttggtgaagattctgctgccagaaaacatgccgttgtgacagttgaccgggagcaagaaacaaggaaactcttacacacttctccagatgacatgctcctttattataaagatccgcaaggtcaaattcagggtcccttttctggtagtgatataattgggtggtttgaggctgggtattttgggatagacttacgagttcgcccagtaaatgccccaaacgacgttgcatttgcttcacttggtgatgtgatgccccacttgcgagctaaagcaaggcccccacctgggtttaatacagcaaagcagagtgaggtacctgatgtgtctagtaggtcaagctcaagtggatttgggaaggtccttcctggtcctagcggggttgatttgcttggaaatgagcatagacatactcatggatcgtcaacagatgctgaaaacaaatttctggagtcattaatggctggagaaataaccacttctcatctggagagatttgcctcctcagaagggctgcaacgatttgttggacaaaattccagtggtatgtctccctcggtggctgaaagtggagatgctctcaaccttttggcccaaagaatgaacctagaacgacaaaaatcattgactcaatcttatccgtactgggcaagtagagaaggagcccctgtgggccctgactttgttaggaactctaccattccacaggcaaatccactttcaactgttggtgatattccccgacaatctcttcctcagaatgttgagatgatgtccattcttcaaggcttatctgataggtcttctggtggagtgaacaatggattaggtggttggccaaaattttctgtgcaaggtggcatcgatccatttaaagaaaacgttgacttgcattccggacagaattttcctcaaatggcatatggtatgcaacagcataagcagcagccacaaaatcatccatcaatgacaaatttacttgcccagactctagacaaccctgccatccagtctgacaagctactctcggctggccttacacatgaccctcaggcattaaatctgttacagcagcagtatttgatgcaattacattctcaacaggctgtcccaggcccacagctttcaattctggataagttcctattgcttaagcagcaacaaaagcaagaggagcagcaacagatgttgcgacagcaacagcaacaacaattgctttctcaggttctgtcagagcagcagcttgttcaacgttttggtgatccagcttttgggcagttacagacttctgcatcgcctattggaaatgcacccattgaacaatctgcgccccagctgtcaccctcattgtttcaagctaattctcaagttcctgtttctagtatgccagatgatagaaatgttagcattgccaatatgcctccaaaattttcccagggtttgggtcatcaagtacctgctgacggatcatctttgcacttgcctcatcagatgttaggaacggcacaaaaaaattggccatctatgtcaacacatgccgacaacattaacaatttaccatcaggttcagaaatgatgggaaaaataaccgcctccgaagtagtggacaagtccctgagtacggctgcagtggaacgtgtcacttctgaacatagtgcaccatctttatcaggccaggtggtagagagaaactcaaaaacagcacaacctactttgattgtggatttggggatgagtgtgctaagtgctgactctgtaccacccaagttttctgaatcatctgtgagccctagggaaaatcagattttgatgtttgagagaacaaatgataataagcttacaccaactggtactctagaggaaaactctgaaaaggagtgtcatgaagatgtatctgcagcaaaagacgcaaaaaatgttgaatcacgtgaggttaagaaaacttccgacaagaaatcaagaaagctaaagtcttccaaggcgcaaccatctcctgaccagttgaagggagcttccaaagcgtctactgtgcagaaatctaagcaatctgaagctgaaggagcacttagtgcgaccaagtcgaatctttctgttgcttccagaagtacattagatggaagacctatggaggcagctagggaaatatctaacccatcagaaattgatctctatacttcgcttaattaccctgctggtgtttttgaatctatggacgccaaaggtgaacagaaacagagtgattctgttacgcaacaaggtgtgcaagtaagttcttcacagagagcttggaaaccagctgtttgtgtgaagcctaagtcactattagaaattcaacaagaagagcagaaaagggcacagatggagatccctgcccctactgtctctgcttctattagtcctatcagtttctccagtccttggggtggaatggttacccatgtggagcctaaagtatctagagatattcaccaggatagtaatagtagtgagcatatggggaaaacagagtcgccaaagagtaagaagagtgggttgcatgatcttttggccgcagaagttttagcaaagtctaacaacagaagcattgaaatcactgacaatgcttcaagtcttcctcttttaccaacaacgcctaatcagttagatgtggttgatcacgagaattttattgaggctaaagataccaagaagagtcgtaagaaagctgcaaaggctaaagcttctggagcaaaggtttcagctgcagttccttatgctgatgtggctgttgcatctggtcctgttgagaaagtaaaaagtactaaacatgtgcaagcagagaaggaggtattgcctgcaccaccatctggtccatctctaggggactttgttccttggaagggtgaatcaactagcccttctccagctcctgcttggtcttcagatgctggaaagtttccaaaacccacatcactgagagacatcctgaaagaacaggggaagaaaggaactccacatcaacagtacgcaccaatatcaacttcaccaaaacctagttcagctcagacgcgtcctgtgtcgtggtcagtttcagcatcatccccttcgaaagccatatctccggttcagatcaattcccatcctccttcacaaacaaagtataaaggagaagatgatctgttctggggtcctccagatcaatcgaagcaagagagcaaacaggcagtctttcctcagcttggtaaccaaggcagctggactactaaaggcactcctgtcaaaggaactcctggaacttctattaaccgacaaaagtctgctggtggcagggctgtggagcacatttcgtcttcacctgtcgctgcacaggtttctgggaaagggaaacgagatgggagtacaaagcacttggaggcgatggacttcagagattggtgccagaatgaatcagttcggcttactggaagcaaagatacaagttttcttgagttttgctcaaagcagtccagatctgaggctgagacacttcttacggaaaatcttggatcttttgatccaaatcatgaattcattgagaagtttttgaattacatggagttgctgtcagcagatgtgctggagcttgcctttcaaagtcgaaatgatctgaaggcctataacaaaggagccagagacatgaattccgactatcaaggtttgagagaccttgacaaagacagagatgttgtggcaagtgctgatgcatcgatgaaaggagggaaaaagaagggaaagaaaggaaagaaggtgagctctgctgtattggggttcaatgttgttagcaaccgcatcatgatgggtgagattcaaacccttgaagattaa

SEQ ID NO: 52 - DNA, 1080 bp, Spinacia oleracea
  source 1..1080: mol_type = genomic DNA
atggattattcgacttcatggttgagccactcatcaccttctctcgatatttctaatgctaacacacttcgccttttcgacgatgcccctgttagagataccaacatcttcaagcaagatccttccccacctcgccctccttctaccaatctttggcacaaagatgagagtggagcattagtagaggaattgaagagggttaatgcagagaacaagagactaacagaaatgctaaccgtagtttgtgaaaactacaatgaattgaagagacagttggtggatcatatgagcaagcagagttcaagtaataatgagattaccaagaaaaggaaactggacaacaacaacaacaacaacaacaacaacaacaataataataacaatggccttgttgttagaactaataataataataatggtgagagtagttccagtgatgaagaggattcctatggaaaacctaaagaagagactattaaagctaagatcacaagggttgctgttcgtactcaagcttctgattccacccttattcttaaggatggataccaatggagaaaatatggccaaaaagttacaagagataacccttgccctagagcttacttcaaatgctcctttgctcctggttgccccgttaagaaaaaggtacaaagaagtctagaagaccagtcaatgttagtggcaacgtatgaaggagagcacaaccaccaaccaccagcacaacaagagggtcagggtggtgcaccatcgggttctagccggtccttgccccttggctcagtcccatgcactcttgacctaaccaagtccaagtccaatgctaacaacgtcgtaaacgaaacaacaactacaaccactagcagcaacaacaacaacaacaacaacaacaacaacaacaaggtgttgaataacacgacgtcgtctaaccataaccagttgccaaagtttagctcaccagaattccaaaaactattggcagagcaaatggcttcatcgttgactaaagatcccaacttcacagcagcgttggcagcagccattaccgggagattcgtccagcaaacttcaagtgattaa

SEQ ID NO: 53 - DNA, 927 bp, Spinacia oleracea
  source 1..927: mol_type = genomic DNA
atggagtcatcatctaatttggttcttgacacttctcttgggttcaatcttaaccttaatctttgtcacaatccggaaatcttcgatgacaaaacacaggtggacgagagaaaaagtgggaatttagaagaggagttgaacaggatgaacatagagaataagaagctgacaaaaatgttggcatccatgtgtaagaattacagtatgctgcaaaatcagttgattggtttgattagtccgacgtcgaaaggcgaaaatgaagatcagaatccaaggaagaggaagtttattgaagctgtggagtgtggaatacaggggaacacagaacagagttgtattagctacgatgattcattcaaaagagtaactcttcttagcagcaagcctaagatcactaataaaatgttgattcggaccgataaatcggatattagcttggtggtgaaagatggatatcaatggaggaaatatgggcagaaagtgacgagggataatccatctcctagagcttactataaatgtgctaatgctcctgaatgtcaagtcaaaaagaaggtgcaaagaagtgcagatgatccatccatcttggtagcaacatatgaaggagaacacaatcatcctcaaccaaattcaagggatcatttagatcaacaagtggtattaagctcaagtcaatcagtaaataataatactaataatgttataagatctcctaatacattaaaccttgatatgatcaaccataattatcccaataacatggagaagacggtcgactacaacgacgtcgacacgaaagcgttgcagagattacttgtagaacaaatggcttcttcattgacaaaagatccttcttttacttctgcattagctgctgccatttctggaaaaatatcgaacaatgcttatactcagagatggttataa

SEQ ID NO: 54 - DNA, 774 bp, Spinacia oleracea
  source 1..774: mol_type = genomic DNA
atgtctagctcgtcggtgattggtgattccggtagatttcccggccaaaagatgagtttctcgcagacttgtaaccttctcagccagtatgttaaggagaaacgcgctcaaggtgttactggaaacatcgagtcccgaggaccaacttcaacgatgaatttgttccctgtgagtgggaaacatgcagctgaagattcaaatgtttgtgctgggaaaacaatcaatctctttcctcagctttctgggtttaattcaagcgtgaacaagcgtgctaaccccgagcctccgactccccaaatgaccatcttctatggagggcaagtcttggtctttaatgacttgccgactgacaaggcaaaagaagtcatggacctagcttgtagctttgaagctagcctcaagaagaggaaagtcgaacctaccagttcgagtccccctagcccggttgcagcccctgttcctgcccaaaaccccaaaccaaccctgaatcagaccctgattccaacccagaaatcaaaccagaacttgactgttgctccaaaatttgtgaagaacgtcataccagctaccgtgcctaatgtttctgatctgcctattgctaggaaggcctcactccaccgtttcctcgagaagagaaaagataggcttgtgggcaaactccccgcggaaattccaaatggaagcccggcaaagcccctagagggtaaggcagcgtggctcggactgaattctgtgccagtgtttcagatgcagaagccgcagcagcaatcctag

SEQ ID NO: 55 - DNA, 369 bp, Spinacia oleracea
  source 1..369: mol_type = genomic DNA
atggattgtaatcttgagcttggcctatcaccatcttccacgtcgatctcttcaacagcttctacgatctccatgggatctgttaatggagataatccaaaaaagcaagaacaacaaaggacgatattttataatggaactatctgcgtttgcgatgttaccgatgttcaggcaaaaagcataatacagttagcaagtagaaaaacgatacaagaaatatgtacatctacaacaataacaccaacgttacaatctcaacaattatgtactcaaacgcaaaatggtttatcggccatgaagaaatcgttacgccaatttctcgaaaagcgaaagcatcgaatccaagctacctcgccttaccgtctttag

SEQ ID NO: 56 - DNA, 648 bp, Spinacia oleracea
  source 1..648: mol_type = genomic DNA
atgtctaaatcgtctcacgttgaactcgatttctttggtttgcacaaccttgccaattccatggctcatcctcctcagcctcagacacaaccgcagccgcagccgcagccacagcctcgccttgatcgccgtcaaagtttccgtggaattgcaaagataaatccggagatagtgaagagtgtgattgcatcagctaatgcttccaagtcctcatcatcacatcccgggactcccacttcaagtcttggaatgtctcaacttcccctctttcatcccaatcctttttctggggaagatagcatggagaaagcagaagaaactgccccaatgaccatattctacaatgggtcagtttctgtttatgatctacccaacgacaaggccgaaagcataatgaagcttgcaatggaaaagaatgaatcaaaaaacgaagggaaggtggtggattattcatgttttaagtcatcccaagcaactactaagaatatcctcggacgacttaatgatgatgatggagatgattctgatatgcctattaaacgtaagaagtcattgcagaggttctttgagaagcgcaaagagaggttggtgtcgccatatacaaacatagaacacatggcgtctggaatagagcagcaaaaggcctaa

SEQ ID NO: 57 - DNA, 1365 bp, Spinacia oleracea
  source 1..1365: mol_type = genomic DNA
atggacgccttagctcctcaaccttccgacaccgacatgatggatgccggagctccaccgcctgaaaatcctcttccacccgcggctgatcattctcagaccccgccgttgcagcaacaacagcaagttagtattccggcgactctcagccatggcggaaagtttatccaatacaatatctttggcaacatctttgaggttactgccaagtataaacctcctattatgcctattggaaagggcgcttatggcatcgtttgttcgtctttgaattcggacacgaatgagcatgtggcgatcaagaagattgcgaatgcgtttgataataaggtggatgccaagaggactttgcgtgagatcaagctacttaggcatatggatcatgaaaacgtcaatattttcccaaaaggtcttggaggttgtctgatgtacatattcactgaattagtgagtggtttacttagagttatatgcaaggttgttgcaatcagagatattataccacctcctcgaagggaggcatttaatgatgtatatattgcatatgagctgatggacactgatcttcatcaaattatccgttcaaaccaaggattatcagaagagcactgtcagtattttctctaccagatactacgtggattgaagtacattcactctgcaaatgttctacacagggatctaaagcctagcaatctcttgcttaatgccaactgtgatttaaaaatctgtgattttggactagctcgagtaacatcggagactgattttatgactgaatatgttgtgactagatggtatcgggcaccagagttattgttgaattcttcagattacactgctgctattgatgtatggtcagtaggttgtatttttatggagttgatggaccgtaagcctctatttccaggcagagatcatgttcatcaattacgcctgctcatggagcatgatgagcatgttcgttattttgttgtcttgaagagccttctgttcttgaaagttctgctgattggaaccccttcagaacacgagctagggtttctcaatgaaaatgctaaaagatatattcgtcagcttcctgcatatagaaggcagtcgttagctgagaagttttcacatgtacatccatcagctattgatcttgttgaaaaaatgttgacatttgatcccagacagagaattacagttgaagacgcgctagcacatccttacctgaattcattgcatgacatcagtgacgagccaatatgtgtgtcccccttttgtttcgactttgagcaacatgctttgtcggaggaacagatgagagaattgatttatcgcgaggctcttgcattcaatcctgaatatcatcaggagtga

SEQ ID NO: 58 - DNA, 2970 bp, Spinacia oleracea
  source 1..2970: mol_type = genomic DNA
atggaagctgagattggaggaaaagctaatatttttggtgtggtggtggctgaccagaagacggtaggaaaaaggactttagaatgggatttgaatgattggaaatgggatggtgatctttttactgccagtccattgaattctacacctttagattgtagaagtaagcagctttttcctcttggaccagaacttgtggctaacaccaatgtctgttctaaagaaattaataaaactaatgagaaagggaaaagggaaatggagaagagaaggaggggtgtggttgttcccgaggaggagttgaatgatgaagctagaccactcaatttgaagttgggtgaacaggtttatcctatagcggaaggtgaagctgataaattggaagggaagaccgtgaagaagagtaagcctaccggaactggatcttcccaacctgcctgtcaggttgaaggttgtagtgctgatcttagtactgcgaaggattaccatagacgtcataaagtttgtgaagtgcactccaaggctactgaagctttggtaggaaatgtcgtgcaacggttctgtcagcagtgtagcaggttccattcgctgcaagaatttgatgaagggaagagaagctgtcgtaggcgtttagctggccataacaagaggagaagaaaaacactttctgaaacttcacctaatgttggctccttggctgatgaaaagagtgcggggtacctactagttagtctgctgagaatattgtccaattcccacacacatgcatctgacgagtcaaaaaatcaggatctgatttatcatcttgtgagaaatcttgctagtcaagttagtggaagcaacatgcctgagctacagcaaggatcccagagtttactaaatggaggaatatctgttggaattccacaaaagaacccttccagtatgcaagaaccatgtttggctgtaccttcagcagcagcacatataggattattgacgagagagaatcggcatgtgacagaacacactcaatatgcttcactaccaggcattttacatcaagctggtggttcagtaccttcaaaaggaaatgagccagaggcttacctcaagagacatgggctcagtggcattgacttgaataatgtttatgatgactcgcaagagcgtatagatattccggaaaagggacatggtcctgttgcttgtgcatttaggccagaacgtttacagaaatcaagtccacctcaaacaagtagaaactcagattcaacttctggtcattctagttctagtggcagtgaaatacagggccgtactgatcggattgttttcaagctgtttggtaaggatccaaatgacttgcctcatcaattgcgaacacagattcttgattggttgtctcacagtcctactgacattgaaggatacatcaggcctggctgtattgttttgaccatatatcttcgtttgaataagtcttcgtgggaggagctctgctatgacatgagctccagcttgagtaggctcctgagtctgtctgatcatccattttggaagacaggatggatctataccagagttcagcaacgtgcagcatttatttgtgatggccgggttgttctagatacacctctacctttcaaaagccgacggagtcgaatctcaagcatcagtcccattgcggttcctgctgatgagacagttcagatagttgttaagggttgtaatctgtctggaccaacttctaggttactctgtgctatagaagggaagtatttggtccaggagagttgttatagcttggagggtatttctactaacacagctgctgagcaggatgagatccagtctctcagttttcattgttctataccaaacgttgttggaagaggatttattgaggtggaagattatgggctcagtggttgcttcttcccaattatagttgcagagcctgaaatttgttcagagatttgcatgctggaaagcgtaatggaggcggttggtacagatgaggatattaatagaaaatgtggcacgacagatggaaggaatgacgccttagactttatacatgagatgggctggctgcttcatcggaatcacttgagcataaaggcaggtcaaactgatctccccttggatctattcccttttgtaagattaaagcggctgattgagttttcaatggaccatgattggtgtgctgttctgagaaaacttctggacttgatgttcagtggaactgtagatactggaaatcatgcttcagttgaacatgcattgtcagagatgcccctcctccacactgccgtccaaagaaatagcaggccaatggtggattttttgttaagatacatgcccaacaaggttaaaggtacaataggctttgaacagaagcagtcagattatggaacacccagcacctacttatttagacctgatgtcactggttcaaacgggttgacccctctgcatatagctgctagttgtgctggtttcgagaatatgttggatgccttgcttgaagatcctggaatgattggtattggagcttgggaaaatgccagagacagcacagggttgaccccaaaagactatgctcgcctgagaggtcataaccattacatggatttgttccagaggaaacttaacaaaacttcatctggcaaacatgtagtggtggatatcgttggactcacaaacatagaatcgaagcaaaagcaatcagataaagtaaaatctgcaaagttgaatagcttgtacacggagatgtggcaagtccgtcagaagtgcaaactgtgcgagcagaaaccaaattatggatcaagaggaacatcattagcatttagaccagcagtgatgtcgcttgttgccattgctgttgtatgtgtttgtacagctctgtttttcaagagcatgcctcaagtttgttgtgtgttcgggccattcagatgggaggcactgaaatatggagcaatgtag

SEQ ID NO: 59 - DNA, 3261 bp, Spinacia oleracea
  source 1..3261: mol_type = genomic DNA
atggaggcgacaggtggaatggtggctggatcttacaaaaggaacgaacttgttagaattcgccatgatagtactgatagtgggtccaaatctttgaagaatttggatggacaaatctgtcagatctgtggggataccgtgggagttacttcgaatggtggtgtctttgtcgcttgcaatgaatgtgcttttccagtttgcaggccttgttatgagtatgagagaaaagatggaaaccagtgttgcccccaatgcaagacaagatacaagagacagaaagggagtcttcgagtgcagggcgatgatgaagaggaggatgttgatgatttggataatgaattcaattatgaaaggggaaccagcaaggctagacatcaatggcagggagaagatgtagatctctcgtcatcttctaggcatgaatctcagccaatccctctccttaccaatggtcaagtggtttctggggaaataccaagtgcgacaccagacaatcaatctgtgagaagcacatcaggtcctataggccctgagaagcgtggtaaccattcccttccttatgttgatccatgcctgccagttcctgtgagaattgtggacccttcaaaggacttgaattcttatgggattggtagtgtggactggaaggaaagagttgaaagttggaaactgaagcaggagaagaatatgactcatacgggaaatcgatattcagaaggaaaagggggcgatgttgaaggctctggttcaaatggtgaagaattccagttggctgatgatgtgcgtcaaccaatgagtcgtattgtccctatcccttcatcacatctcactccttaccgagctgtgatcattttccggcttatcattttagtcttcttcttgcaatttcgtattacccatcctgtggaggatgcatatccgctatggctcacatcagtcatctgtgagatctggtttgcaatgtcatggattttggatcagtttccaaaatggtatcctatcaaccgtgagacgtaccttgacaggctcgcattcaggcatgaccgcgaaggagagccgtcgcagttagctcctattgatgtgtttgtcagtacagtggatcctttgaaagagccgcctatcatcacagctaataccgtgttgtccattctagcagtggactacccagtcgacaaagtttcttgctatgtttcagatgatgggtctgcgatgctgacatttgaaggtctttctgaaactgctgagttcgccaggaaatgggtgccattctgtaagaagttcagtattgagccaagggcaccagaattctactttcaacagaagattgattacttaaaagacaagattcagccttcttttgtcaaggaacgcagagcaatgaagagggagtacgaagaatttaaggtccgaatcaatggccttgttgcaaaagcacagaaagagcctgaagaaggatggacaatgcaagatggaacagcttggcctggaaataaccccagggaccacccaggaatgatccaggtgtttttgggccacagtggaggtcttgacatggatggaaatgaattgccacgattagtttatgtttctcgtgaaaagagacctggcttccaacatcacaagaaagctggtgccatgaatgctttgatccgtgtttccgcagtactgacaaatggtgcatatatactgaatgtcgattgtgatcactacttcaacaacagcaaatgtcttaaagaagccatgtgtttcatgatggatcctgctcttggaaagaaagtatgctacgtgcagtttccccaaagattcgatggcattgatttacacgatcgatatgctaacagaaatattgtgttctttgatattaacatgaagggtcaggacggcatccaagggccagtttatgtgggaactggatgttgtttcaacagacaagcactttatgggtatgatccagtattgactgaagaagatttcgagccaaatttcatcatcaaaagttgttttggatcaaggaagaaggggaagagtggtaacaaaaagtacatggacaagaagaggggacctaaaagaagtgaatcttccataccaattttcaacatggaagacatcgaagagggtgttgaaggctatgaggatgagaagtctcatcttatgtctcagaagagactagagaagcgatttggtcaatcaccagtgttcattgcagctaccttcatggaaatggggggtattccacctacaactaatcctgccacactactgaaagaagccattcatgttattagctgtggatacgaggacaagtctgaatggggtaaagagattgggtggatatatggttcagtgacagaagatattttgactggatttaagatgcatgctcgtgggtggatgtcgatctactgcatgccaccaaggccagctttcaagggttcagctcccctcaatctttctgatcgtttgaaccaagttcttcgatgggctctaggttctattgagattatgcttagcaggcattgccccgtttggtatggctacaagggaaggttaaggtttttggagagattggcttacatcaacactgtcgtttatcccctaacgtctattccacttattgcctactgtattctcccagctatctgtctattgaccaataagttcatcattcccacgctaagcaactttgctagtatactgtttatcatgctcttcatgtccattgctgcaacgggtattcttgagctgagatggagtggagtatccatagaagactggtggagaaacgagcagttctgggttattggaggtacatcagcccatctgtttgccgtgttccaaggtttgctaaaggtgctagccggtatcgataccaacttcaccgtcacttccaaggcagccgacgaagacggtgattttgcagaactttacatatttaaatggacggcgcttcttatccctcccaccaccgtcctgatcgtgaacttggttggtgtagtggcgggtgtttcttacgccattaacagtggttaccagtcatggggcccactgtttgggaaactgtttttctcattctgggtgattgctcacttgtatcccttcctcaagggtctattgggtcgtcaaaacaggacaccaaccattgttattgtttggtccgttcttctcgcttccatcttctcgttgctctgggttcggattaatcctttcaccactgatgctgagaaggctgcagctggtaatcaatgtggtatcaattgctag

SEQ ID NO: 60 - DNA, 3183 bp, Spinacia oleracea
  source 1..3183: mol_type = genomic DNA
atgatggaggattcacaaagtggggtgaagcccacaaagcaagcaaatgaacaagtctgccagatctgtagtgacaacatcggcacaactgtagatggagagccgtttgttgcatgtgatgtttgttcctttcctgtttgtcgtgcttgttacgagtatgagcggaaagatggaacccaatcttgcccacaatgcaagacgagatacaagaggcagaaaggtagtccagcaattcatggagaaaaggtggaagattctgatgttgaggatgtggtcagtgacgtaaatgagcccttgggaagttcaatcctaaaggagaagcaacaggagagaatgcttggatggcatatgaatcatgggcaatctggtgagcttggcccccctacctatgataaagaggctcctattagtcatatccctcggcttgctaccggacgcacggtttctggagacctgtcagctgcatctcctggacgtttttcaatgccatcccctggtgccagcactggagctaatattagagtgtcaagggaattcgcatcgcctggatttggaaatgtagcttggaaagaaaggattgatggttggaagatgaagcaagagaagagtacaggtcctcctagtgttagtcatgcgccttcagaaggaaggtttgccaatgatattgatgccagcactgaaattgctatggacgatcctttactaaatgatgagacacggcagccgttatcaaggaaggttcccattccttcatctagaattaatccgtacaggatggttattgttctgcgacttgctgttcttggtatctttttgcactaccgtgtaacaaatccggtgcctaatgcctatgcattgtggctgatatccgtcatttgtgagatatggtttgcattttcatggattctggatcagtttcccaaatggcttcccatcaatcgcgagacatatcttgacaggctggccctcaggtatgatcgtgaaggggaaccatcacagctggccgctgttgatatatttgtcagtactgtggatccgttgaaggaacctccccttgtcactgccaatacagtgttatccattcttgcagttgactatcctgtcgacaaagtttcttgctatgtttctgatgatggagctgctatgctgacatttgaagctctttcagagacatcagaatttgcaagaaagtgggttcctttcacaaaaaaatataatattgagccacgagctccagaatggtacttctctcagaagattgactacctaaaagataaggttcaaactacgtttgtgaaagaccgaagggctatgaagagagagtatgaagagttcaaaatccgaattaatggccttgttgcaaaggcaacaaaggtcccagaggaaggatgggtaatgcaagatgggacaccttggcctggcaacaataccagagaccatccagggatgatccaggttttcttaggccaaagtggaggacttgacactgatggtaatgaacttccacgtttggtttacgtatctcgtgaaaaacgtcctgggtttacacatcacaaaaaagctggtgctatgaattctcttgttcgtgtatcagcggttctcacaaatgggcccttcatgttgaatcttgattgtgatcattacataaacaacagcaaggcgttaagagaagctatgtgttttatgatggatccaaaccttgggaaatattgttgctacgttcagtttccacagagatttgatggaattgataggaatgatcgatatgctaaccgtaacactgtcttctttgatattaatttgagaggtttagatggtatccaaggacctgtctatgtgggtacgggatgtgtcttcaacagaactgctttgtatggttatgagcctcctatcaaaccaaagcccaagaagaaaggtatcctatcatcatgctttggtggatctcggaagaaatcaagcaagaaagactccaagaagaaatcaaaacatgctgaccctactgtgccaatatttaatctggaagatatcgaggaaggagttgaaggtgctggatttgacgatgaaaagtcactactaatgtcacaaataagcttagagaaacggtttggtaaatcagaagtatttgtggcttcgacgctcatggaaaatggcggtgttccacagtctgcaacccctgacaccctcctcaaagaagctattcatgttatcagctgtggttacgaggacaagaccgattggggagctgaaattggttggatttatggttcagttacagaagatattcttacgggatttaagatgcacgccagaggatggcgttcaatttattgcatgccaaagctcgcagcttttaagggttctgctcctatcaatctgtcagatcgtttgaatcaagtgcttcgatgggctttaggttctgttgaaattcttttcagtcgtcattgccccttatggtatgggtatggtggacggttgaaatggctggagagatttgcttatatcaacaccactatttaccctcttacatccattccacttcttgcatattgtaccttgccagctgtctgtctccttactggaaagtttattatccctcagattagtaacttggcaagtgtatggtttttatccctgtttctttccatctttgcaaccggtatcctggaaatgagatggagtggtgtgggaatagatgaatggtggaggaatgagcaattttgggtgattggagggatatcagcccatctgtttgcagtattccaaggtttgctaaaagttctagctggaattgataccaactttacagtgacttcaaaagcatcagatgaagatggtgatttcacggagctctacttgttcaaatggacaactctgcttatcccaccaaccactattctgatagtaaatcttgtagcagtggttgctggaatctcatatgcaataaacagcggttaccaatcatggggtccactttttggcaaactattctttgcattttgggtgattgtccatctttatcctttcctaaagggtctcatgggtcgccagaatcgaactcctactattgttgttgtttggtcgattctacttgcatccatcttctccttgttatgggttagagttgatccctttacaacccgggtgactggtccagacgttcatatatgtggaattaattgctag

SEQ ID NO: 61 - DNA, 3261 bp, Spinacia oleracea
  source 1..3261: mol_type = genomic DNA
atggaggcgactggtggaatggtggctggatcttacaaaaggaatgaacttgttaggattcgccatgatagtactgatagtgggtccaaatctttgaagaatttggatggacaaatctgtcagatctgtggggatacagtgggagttacttcgaatggcggtgtctttgtcgcttgcaatgaatgtgcttttccagtttgcaggccttgttatgagtatgagagaaaagatggaaaccagtgttgcccccagtgcaagacaagatacaagagacagaaagggagtcttcgcgtggagggagatgatgaagaggaagatgttgatgatttggataatgaattcaattatgaaaggggaaccagcaaggctagacatcaatggcagggtgaagatgtagatctctcgtcatcttctaggcatggatctcagccaattcctctccttaccaatggtcaagtggtttctggggaaataccaagtgcgacaccagacaatcaatctgtgcgaagcacatcaggtcctataggccctgagaagcgtggtaaccactcccttccttacattgatccatgcctgccagttcctgtgagaattgtggacccttcaaaggacttgaattcatatgggcttggtagtgtggactggaaggaaagagttgaaagttggaaactgaaacaggagaagaatatgactcatacgggaaatcgatattcagaagggaaagggggtgatgttgaaggctctggttcaaatggtgaagaactccagttggctgatgatgtgcgtcaaccaatgagtcgaattgtccctatcccttcatcgcatctcactccttatcgagctgtgatcattttccggcttatcattttagtcttcttcttgcaatttcgtattacccatcccgtggaggatgcatatccattatggctcacatcagtcatctgtgagatctggttcgcaatgtcatggattttggatcagtttccaaaatggtatcctatcaaccgtgagacgtaccttgacaggctcgcgttcaggcatgaccgtgaaggagagccgtcgcagttagctcctattgatgtgtttgtcagtacagtggatcctttgaaagagccgcctatcatcacagctaatactgtgttgtccattctagcagtggactacccagtcgacaaagtttcttgctatgtttcagatgatgggtctgcgatgctgacatttgaaggtctttctgaaactgctgagttcgccaggaaatgggtgccattttgtaagaagttcagtattgagccaagggcacccgaattctactttcaacagaagattgattacttaaaagacaagattcagccttcttttgtcaaggaacgcagagcaatgaagagggagtatgaagaatttaaggtccgaatcaatgcccttgttgcaaaagcacagaaggtgcctgaagaaggatggacaatgcaagatggaacagcctggcctggaaataaccccagggaccatccaggaatgattcaggtgtttttgggccacagtggaggccttgacatggatggtaatgaattgccgcgattagtttatgtttctcgtgagaagagacctggtttccaacatcacaaaaaagctggtgccatgaatgctttgatccgtgtttctgcagtactgacaaatggtgcatatatactgaatgtcgattgtgatcactacttcaacaacagcaaatgtcttaaagaagccatgtgtttcatgatggatcctgctcttgggaagaaagtatgctatgtgcagtttccccaaagattcgatggcattgatttacacgatcgatatgctaacagaaacattgtgttctttgatattaacatgaagggtcaggacggcatccaagggccagtttatgtgggaactggatgttgtttcaacagacaagcgctttatgggtatgatccagtattgactgaagaagattttgagccaaatttcatcatcaaaaattgttttggatcaaggaagaaggggaagagtggtaacaaaaagtacatggataagaagaggggacctaaaagaagtgaatcttccataccaattttcaacatggaagacatcgaagaaggtgttgaaggctatgaggatgagaagtcccttcttatgtctcagaagagactagagaagcgatttggtcagtcaccggtgttcattgcagctaccttcatggaaatggggggtattccacctacaactaatcctgcgacacttctgaaagaagccattcatgttattagctgtggatacgaggacaagtctgaatggggtaaagagattgggtggatatatggttcagtgacagaagatattttgactggatttaagatgcatgctcgtgggtggatgtcgatctactgcatgcctcccaggccagctttcaagggttcagctcccctcaatctttctgatcgtttgaaccaagtgcttcgatgggctttagggtctattgagattatgcttagcaggcattgccccatatggtatggctataagggaaggttaagattcttggagagattggcttacatcaacactgtggtttatcccctaacgtctattccacttattgcctactgtattctgccagctatctgtctattgaccaataaatttatcattcccacgctaagcaatttcgctagtatattgtttatcatgcttttcatgtccattgctgcaacgggtattcttgagctgagatggagtggagtatccatagaagactggtggagaaacgagcagttctgggttattggaggtacatcagcccatctgtttgccgtgttccaaggtttgctgaaggtgctggccggtatcgataccaactttacggttacttccaaggcagcggacgaagatggtgattttgcagaactttacatatttaaatggaccgcacttcttatccctcccacgacggtcctgatagtgaacttggttggtgtagtggcgggtgtttcttacgccattaacagtggttaccagtcatgggggccactgtttgggaaactgttcttttcattctgggtgattgctcacttgtatcccttcctcaagggtctattgggtcgtcaaaacaggacccctaccatcgtgattgtttggtccgttcttcttgcttccatcttctcgttgctttgggttcggattaatcctttcaccactgatgctgagaaggctgcagctggtaatcaatgtggtatcaattgctag

SEQ ID NO: 62 - DNA, 1737 bp, Spinacia oleracea
  source 1..1737: mol_type = genomic DNA
atgggaattcacaattccccttctctctctctccaacctgccgatgaacaacctcacatcatcaccaccgttcatcacactccctctcttaacccaccaccacctgaaaccccttctcagccatgcgatttcccctcgaaaccccaaaccagcggcttgaaagtatcccggaagatcaagaaacgtgcccttgatgaaacctcctctgcttcgtcgtcttcttcaattcaaagaagaattcgaattcctaataaacgcagaaaccctaggttttctgttcgtcgtagcgcctccgatgtcgaagccattgctttccctcttgggatgtcgattgccgctgttttggctcagattttggagaagaaggacttgacgactgagaatgtttccattgatcatctggcgatggtatttggcgacaagtttgatgcttttgcaagaaacttcgagaaatcttttggaagtaccctcagcactctgactttgataaataactcatctatagacaataaagatgatcacgttaggaataacattgttggcgattgtatttctgatgcaacccaatccatctcctttaacaaaggagattgttccgcaagaagtagtgatgttatagaggattgcagaagatcaataaatggcgctactgggcctcagcgtcgtataaaaatggttgaagaggtagaagatattcagtcagcagactctaggattcaggagcgttccctgcaagggcgtataagctcacaaattgttgtttcaaatatgggaaatcacttgagcaactattcagtgctcagtaccattgagaaatctgttgttgagcagacgcgtgcaaatgacctaaagagcgttgaaataggtctaattatgaaaaagttgcggctgaaggaaactcaactggctcttaattctgatttgaatttgttggagagatgtaaattgtctctaggaatttcaagaacatccttcaaagtcgagaagtttaagaatcaattggaagagacaagacatagtgagctcctcagaacatgtgcagactttcttgttgctgatttgcttataatgtcagggtgcctttgctatggagtgtatgccttttcatatgacaatcttcgccaactcagcagatcatgttctgcttccgaggtgtccaagtcctggtggatcccgaaaccaatggcctcatttaattcaggcctccagaccctgaggtgtgaggttcaagtatacagcaggatactttttggtttgctgatgattttagcaatcgcatatattcttatccaacgctctgaaatttcgagacaagccatgccaatcacttttctaatactgcttttaggggttggctgtggttttgctggaaagctttgtgtagatactttaggaggtgatggatatcagtggctcatatactgggaagttatatgcatattgcacttcttcatcaatgttttcacgcctttcttattttatatccttaacgggcccatccaagtatctcaagagaaaaagtctcgctccatcttcccgttttggataaggcgctggttgttctatatcattctcattttgattccactttgttgtggtcttttgcctttcgcgagcccaagaacatggtggggagagcacttctcgaaattgatcagaaatcatatgttagctgcctatgatgacgcttatggtgacgggaatgatttccccgatcatagtgcaggtgaactgtga

SEQ ID NO: 63 - DNA, 1800 bp, Spinacia oleracea
  source 1..1800: mol_type = genomic DNA
atggcgaacaacaataacaatcactacaattatcgaagaagtatgtcagatgaaatcttggaatgtatactaccgtatattcaagatcctcgggatcgatcctcaatatcatcggtttgcaggcgatggtatgagctagatcggttgaccagacgacacgtcacgattgcgttgtgttacgcaaccacgcctctacagctccatgatcggtttccgaacatcgagtcgttgaagctcaaaggaaaacctagagcttccatgtataatttgattccggaggattggggtggttatgtgggaccctgggttcagactctgcccagatttcctcgccttaataagctccattttaggcggatgattgttacggatgaagatcttgggagtattgccaccgcaaaaggccgtaatttacagtgcttgaagttggataagtgctctggcttctccacggatggtctcttgcgtatcactagctcttgcagaaacctgagaacactgtttctggaagacagtggtgtcgaggagactaatggtgaatggtttcaccagctggcactacacaatatagttcttgaaactctgaacttctatatgacttttatggagaaaatcacagttgaagatcttgaacttcttgccagaaattgtcctttagtctctgtgaaaattggagatactgatcttgttcatcttggaaaatttcttcgtacagcgacgagattgcaagagttctccggtggttcttataacgaagatttagaccaatatggtaatattgtatttccagctcaattaacttcattgggtcccatgttcttggtgaaaagtcatttgcctgtatattatccaattgcacactcgctgcggaggttggatcttgtttatgcatcgcttaacagtgaggggcactgcgatctgatacgtagatgtcaaaatttagaagttctggagacgacaaatgtaattggtgatcagggtttggaagttgtatctttgacgtgcaaaagactgaagagactacgtatagaacgagggccagagccagatgaacgagggccagaggcagttgagcaaggactggaccctgaaccaggtgtggttacacagacgggattaattgctgtagcacaaggatgccctgacctagaatacctagccgtttatgcgaccgatataacaaatgaagccttggaatgtgtaggtgcaaacttaaaatatttgactgatttccgacttgtattacttgacagagaagaaacaatcactgatttgccactggataacggagtccgagctctattacggggttgtcaaaatatgcaacggtttgcgttgtacctcagacaaggaggtttgactgacactggtctggggtacattggtgagttcagtaagaaagtgaggtggatgcttctaggttatttgggggaaactgatgcaggtctaatagaattctcaaagggatgccctagccttcagaaactagagatgagggcttgttactttacagaacatgcactggctactgcagcaatacaactgccttctctaaggtacatgtgggcccaaggataccgagcatctcaaacggggtttgttgatatagtacaaatggttcgaccatattggaacattgagctgattcctggggatcagtacaatcacaacagcgacgacgacaatggccagcctgctagggttgaccatccatcccatattgttgcttattactcgcttgctggacagagaacagatcatcctgctactgtttaccctctgcccctgccggaaccctag

SEQ ID NO: 64 - DNA, 936 bp, Spinacia oleracea
  source 1..936: mol_type = genomic DNA
atggtttccgaaagtctccccaagcttcctacagttgagctaaagggtaagttgaaagcaggaacaagttcatggacatcaacatgcaacaaagtgaagcaagcactagaagaatacggttgcttcgtggctgtgtatgacaagtttaccgtagatctccacaacaaggcggttgatgttgtacaacccatgtttgaactccctaccaaagttaaagctagaaactcctccgatcttccctaccacgggtattataagcccggaaaacccatgcctcttctcgaaagtctaggcattgaggaggcaacaacgcttcataacatccaatctttcactaatctcttgtggcctactaggaacgatcatttttgtgaaaccatccatgaatatgcaaagacagtatcggagctagaacaattggtaatgaggatggtatttgagacttacggggtggagaaataccttaactctcatctcaactcgaccacttaccttcttcgtatagcaaagtacagggtaccactcaaggatgaacttaatcttggtgcagttggtcacactgacaaaaacttcataacaatacttcatcagaatcaagttaatggtttggaagttcaaactaaagatggtgaatggcttacttttgagtcctcgcctcagtcttttgttgtcatggcaggggagcctttcttggcatggagcaataacagaatacatgcacctctacatcgcgtgatgatgaagggagacaaggcaaggtactcatttgcgttattctcaacaagtaaagacaacattgttacaccaaaggagttggtcgacgaagatcatccattacaatttaagccattcgattatatgggattgcttcgattttataccgaagacgttagtcgaatggccgtctgtaccatcaaagactactgtggcgtttaa

SEQ ID NO: 65 - DNA, 1122 bp, Spinacia oleracea
  source 1..1122: mol_type = genomic DNA
atgggtgatgatgatattgtggcgtcggttcgctctgtggtgttcaaggagtcagaaaccctagaagggaaatgcattaagattgagggttatgattttaatcaaggagtgaactatccccagctttttaaatcgttcatctccaccggtttccaagcatcgaatctcggagaagctattattgctgttaatgaaatgctagattggaggctggcaaatgagatagcagctgaagattgcagtgaggaagagagggatcctgtttttagggagtctgtgaggtgcaaaatattcttaggttttacgtcgaatcttatctcctccggtgtccgtgacactgtacgatatcttgttcaacatcatatggtcgatgttgttgtaactactactggtggtgtagaagaggatcttataaaatgtcttgcacctacatttaaaggtgatttttccttacctggagcgcaattacgctcgaaaggattgaatcgcattggtaacttattggtcccaaatgacaactactgcaaatttgaggattggattgttccaatttttgataaaatgctggaagagcaaaatacagagaagataacatggacaccctcaaagttgatcgctcgtttgggaaaagaaataaatgacaagagttcataccttttttgggcctacaagaataatattccagtattctgcccaggtttaacagatggatcacttggagacatgttgtattttcattcaatacggaatcccggtttaattgttgacgtagttcaagatataagagccatgaatggtgaggctgttcatgcgagtccaaggaaaaccggcatgataattcttggaggagggttacccaagcatcacatttgcaatgcaaatatgatgcgaaatggggcagattacgcagttttcatcaatactgcacaggagtatgacggcagtgattctggtgctcgtcctgatgaggccgtctcttgggggaaaatccgaggttctgctaaaaccgttaaggtgcattgtgatgccactattgctttccctctattagttgcagaaacctttgctgctaaaagagagagagaagctaattcaaaagctgacataggaggaagattttga

SEQ ID NO: 66 - DNA, 288 bp, Spinacia oleracea
  source 1..288: mol_type = genomic DNA
atgtttcagatgaggttacaattccgatcatatgatcctatccaacctatctcattcgttctactacaaaggaatttcaggtgtagaaatatgggtgatagtgatattttggcgtcggttcgctctgtggtgttcaaggagtcagaaaccctagaagggaaatgcattaagactgagagttatacccagctttttaaatcgtttgtctccaccggtttccaagcatcgaatctcggagaagctattgatgttgttaatgaaatgattgacgactggaaaatgagatag

SEQ ID NO: 67 - DNA, 480 bp, Spinacia oleracea
  source 1..480: mol_type = genomic DNA
atgtcggacgaagaacaccacttcgaatccaaggctgatgccggagcttctaagactttcccccagcaagctggtactatccgcaagaacggttacattaacatcaagaatcgtccttgcaaggttgttgaagtttctacttctaagactggcaagcacggacatgctaaatgtaactttgtcggtatcgacatcttcactgggaagaagcttgaggatattgttccatcttctcacaattgtgatgttccccatgtcacccgtactgattaccagctccttgacattactgaggatggttttgttagcctgctgactgataatggagatactaaggatgacttgaagctgccaactgatgaagttctgaataaacagatcaaggatggatttgctgaggggaaagacctgattgtgagcgtgatgagtgccatgggagaagaacaaattaatgccctcaaggacatcggacctaagtaa

SEQ ID NO: 68 - DNA, 486 bp, Spinacia oleracea
  source 1..486: mol_type = genomic DNA
atgtctgacgaagaacaccagttcgaatcgaaggctgacgctggcgcctcaaagacctacccacagcaagctggtaccatccgtaagaacggttatatcgtcatcaagaatcgcccttgcaaggttgttgaagtttccacttctaaaactggcaagcacggtcatgctaagtgtaactttgttggtattgacatcttcaatgggaagaagcttgaagatattgttccatcttctcacaattgtgatgttccccacgtcaatcgtaccgattatcagctcattgacatagctgaggatggttttgttagcctgctgactgagagtggaggtacaaaggatgacttgaagctgccaactgatgaaactcttcttaagcagctcaaggaaggatttgcggatggaaaagacctgattgtgagtgttcagaatgccatgggagaagaacaaatttgtggcgtcaaggatattggtggtggatctaagtag

SEQ ID NO: 69 - DNA, 480 bp, Spinacia oleracea
  source 1..480: mol_type = genomic DNA
atgtctgacgaagagcaccagttcgaatccaaggctgacgccggtgcgtccaagactttccctcagcaagctggtactatccgtaagaacggttacatcgtcatcaagaatcgcccctgcaaggttgttgaagtttccacttctaagaccggcaagcacggccatgccaaatgtaactttgttggtattgatatcttcaatgggaagaagctcgaggatattgttccatcttctcacaattctgatgttccccatgtcactcgtactgattaccagctccttgacataactgaggatggctttgttagcctgctgactgacaatggagacactaaggatgacttgaggctgccaactgatgatgctctgctgaaacagcttaaggacgggtttgccgaggggaaagacctgattgtgagtgtgcagtgtgccatgggagaggaacagatctgcggtctcaaggatattggacccaagtag

SEQ ID NO: 70 - DNA, 2226 bp, Spinacia oleracea
  source 1..2226: mol_type = genomic DNA
atggagtcgtgcaattgtattgagccgcaatggcctcatgatgatctgttgatgaagtatcaatatatttctgattttttcatagcccttgcttatttttcaatcccattggaattaatttactttgttaaaaaatcagctgtttttccgtatagatgggttcttgtgcagtttggtgcttttatagtgctttgtggagcaacacatctaatcaatttatggacatttagaatacatactaaaacagttgaagtagttatgactaccgctaaggttttaaccgctgcagtgtcgtgtgcaactgcactgatgctcgtgcatataattccagatttattgagtgtgaaaactagggaattgtttttgaaaaaaaaggctgcagatctggaccgagagatgggcctcatccgtacccaagaagaaactgggagacatgttaggatgctgactcatgagatcaggagtactcttgaccgacatacaatccttaaaaccactcttgttgagcttggtaggacattggctcttgaagagtgtgctctatggatgccaacgcgtacaggattggaacttcaattagcttatacactccgtcaacaaaatcctgttggatttaccgttcccatacaacatccagtaatcaatcaagtattcagttccaatcatgctgtgcaaatctcaccaaactgtcctgtagctagaatccgtcctatctctacgaagtacatgcctggagaggtggttgctgtgcgtgttccccttcttcatctttctaatttccaaatcaatgattggccagagttatcgacaaaacgttatgcgctgatggttttgatgcttccttcggacagtgctagacaatggcatgttcatgaattggagcttgttgaagttgttgcagatcaggtagcagttgccttgtcccatgcggcaattttggaagaatctatgagagctcaacaacaacttttggagcaaaatgttgcacttgatttggctcgcagagaagcggagactgccatccgtgctcgaaatgactttttggctgtgatgaatcatgaaatgcggacacctatgcatgcaattatagccctttcctccttactgcaagaaactgagttgatccctgagcagcgattgatggttgaggcaattctaaaaagcagtaatcttcttgcgactctgataaatgatgttttggacctttcacggcttgaagatggaagtttgaagcttgataatggagtatttaatctccacacgatgtttagagaggtccttaatttgatcaagcccatagcatctatcaaaaagttgttgataacgacgaatttggcaacggatgtgccagagtttgctgttggtgatgaaaaacgtctaatgcaggttctattgaatgttattggtaatgctgtgaagtttacgaaagaaggaagtgtctcagttactgctttagttgcaaaagcagaatctttaagagatgctcggactcctgatttcattccattagcgacagatgatcacttctatttgcgagtgcaggtgaaggattcaggacctggaatcaatccgcaagatattccaaagctcttcacaaaatttgcccaaccacaaacacctgccaatcgaaattctggtggtagtggactgggccttgccatctgcaagaggtttgtaaatctcatggaagggaatatctggattgaaagtgaaggtcttgggaagggttgcacagctatttttgtagttaaacttggaagtcccgagcgttcaaatggagccatggtcccttttgcatccaaaccattggtgaatcatattcaatcgagctttggtggcctcaaagttctcattatggatgagaatggggttagcagaacagtgacaaaaggacttctgatgcacgttggatgtgatgtcttgactgtagcgtctagtgaggaatgtttgttagctgtctcccgagaatacagggtggtattccttgatttgtctatgcctggatttgatggctttgaagttgctgttcaaataagaaaaaggttctcaaagcgtcatgaaaggccgttaattgtagcactcactgggagtgcggacaggttgacccgagaaaattgtgtcagggtgggtatggatggttgcgtattgaaacctatatctgttgataaaatgagaagtgttttatctgagctcctagaaagacgaactgtgtttgagccgctctag

SEQ ID NO: 71 - DNA, 2235 bp, Spinacia oleracea
  source 1..2235: mol_type = genomic DNA
atgatgagactgatagccattggattgttggtttgcactctcatattatcttcatctaccattggttgtggatattcccattgtaattgtgaggatgaagggttttggagcattaatatcattatggaatgccaaaaagtgagtgatttcttaattgccattgcttatttttcgatacccatcgagctcgtgtattttattagctgtgcaaatattcccttcaaatgggtccttgtcgaattcattgctttcattgttctgtgtggaatgacccacttgctcaatggatggacttactatggtcctcactcatttcaactcatgatgtcacttacaatatttaaattcctaacagctttggtgtcatgtgccacggctataacccttttaactttgatccctcttttacttaaactgaaagttagagaggtgtttttgacgcaaaatgtcttggaattggaccaagaggttgggatgatgaagaaaaagaaggaagctagttggcatgttcgaatgctcactcgagaaataagaaaatcactcgataagcttactatattggatactactttggttgaactttccaatatattagacttgcagaattgtgctgtttggatgccaaataatattaagactgcaatgaacttgacccatgagctgaagcctagcccctcaaatatctttccccgaaatatcccaatggatgatcatgatgttcaggagattcgtgatagtgagggagtgaggattttaagaccggaatcagcactgggagtggcaagcaggggtggttcggaagattctggtactgtagcagccattcggatgcctttgctgaatgtttcaaattttaaaggtgggactcctgaattagtacagacatcttatgccatactagttttggttcttccaagctcaactgatagggtttggagccatcatgaactagagatggtggaagtggttgctgatcaagtggctgtggccctttctcatgctaatgttcttgaagagtctcagttgatgagagatcagctgaaggagcggaatcgtgttctgcagcaggctaggaaggacgcgatcatggcaaaccaagcaagagattcattccagaaggttatgagccatggaatgaggaggccaatgcactcgattctcggtcttctttcaatgtttgaagagaatatgaattttgagcagaaaattatggttgacaccatggtgagaactggtaatgtcatctcaactttattaaatgatgtgacagaaatatctgcaaaagacaatggaaggttccctttggagatgaggctgttccacttgcattccatgattaaggagacttcttgcttggccaagtgtttatttgtgtatcaagggttcagtttttctcctgatgttcagagtagtttgcctgctcaggtgattggtgatgagaggagaacttttcaggttttactacacatggtagggcacttactgcatttttcaaacaaagggggtttgatgatttttcgtgtgtctgccgaaaatggcagtgtgggaaagaatgataagtgggggatgtggaggtcggctactgtcgaggagtacattaaagtaaagtttgagtttgacataagtggaggttatctgacagacactattaggagaacccagaatgattcaaacaaggaagtattgagcttcagcatgtgcaaaaggcttgctcagatgatgcagggtgatatatggatatctctaaactcaaatgggataccacagaacatgactcttgttctacgatttcaacaacaaagatatattcctgaaggaaacttatatatgacagagcacacagtttcttcaaactctcggttcagagggttgaaagtgttgattgctgatgatgataatgtaaatcggacagtgacaaagagactgctagagaagttagggtgtgaagtatctgctagttgttctggttatgagtgcttaactgcattggctccctcacaaaacacttatagggttgtgctacttgaccttcagatgcctgaaatggatggctttgaagtcgcaaggagaatccggaaattatatagccggagtggtccgtctattatagcaataacagcaagtgcagaggataatatatgggagagatgtttacaaattgggatgaatggtctaataaggaaacctgtggtcctgcagggaataatcgacgagctccgaagggtcctcagatga

SEQ ID NO: 72 - DNA, 2295 bp, Spinacia oleracea
  source 1..2295: mol_type = genomic DNA
atgttgagaccactggctgctgctctcttcttttctgctctgttgatatcagcttctgcctctgacaatggctacccaagatgtaattgtgaggatgaagggttctggagtgttgagagcattctagaatgccaaagggtgagtgatttcttgattgctgttgcctatttttcgatccctattgagctgctttactttgttagctgttcaaatgttccttttaaatgggttctctttcaattcatagccttcattgttctttgtgggatgacccatttacttaatggttggacttatggtcctcacgcctttcaactcgccgtagctctcaccacttttaagtttttaaccgctctcgtctcttgtgctactgccattacccttatcaccctcatccctttgcttctcaaagtcaaggtcagagaattcatgttaaagaagaagacttgggatttgggtagggaggttgggatgattaagaaacaaagtgaagctggatggcatgttaggatgcttactcatgagattcgcaagtccttagataggcatacaatcttgcacacaactatggttgagctctctaaaaccttggatttgcttacttgtgctgtttggatgcccaatgagaataggactgagatcattttgactcatggtttaaaaggaaggaattttcagagtttcagcaactgcagagttccctatgctgatcctgatgtcagaaaaatcaaggagagtaatgaggttcagatactcagaccagattcagctcttggtgttgccagtagcggtgggttggaaaattatggtggtgtggctgcaatacgtatgccgatgctcaaggtctcaaatttcaaaggaggaacccctgagctgatgcaggcttgctatgctatattagtcttggttctcccttgtgggcaacagagatcttggaccaaccaagagcttgagattatcatggtggttgctgatcaagttgctgtggctatctctcatgctgccatcttagaggaatcacagctcatgagagatcaattggcagagcagaatcgcgcgctgcagcaggcacaacgaaacgcattgatggcaagtcaagcgaggaactcgtttcagagagttatgagccatgggatgagaaagccaatgcactcagtcttggggttgctttcagttctaaaagatgagaatttaagcagtgaacagaaacttattgttgacacaatgatgaagacagggaatgtcctttctcttttgatcaatgatgtcatggacgattcaccaaaagagagtggaagatttccatcagattttagatcatttgggctccgtgccttggtaactgaagctggttgctttgctaagtgcttgtgtgttcaaaaaggttttggctttagtattgaagttgacagatccttgcccgatcaagtattgggtgatgaaaaaagggtatttcaggtgatcttgcatatgattggtaacttggtggagaacaataccggtggtgggtctttgttttttcgggtctactctcaacatggaagtcagggaaggaatgatcaaagatgggcaacatggcgatcaggctcttctgatggatatgtttatatccgatttgaagttgagataaccaataatgctatcttctcagatgattcagtgttggcttcaaactacagtggtccgagatgcagcagtgagattgtggaccgggacctgagtttcagcgtttgccagaagctagtccagctgatgcaaggaaatatatgggtagttccaaatccacagggttttcctcaaagtatggcactggtactacgtttccagctacgtccatcaattggaataacaatctcagaatcaggagaaccatcatcagataactcctcctccaactttactttaaagggacttcaaatactattagcagatgatgatgacatcaacagggcagtgaccaaaaagttgctggagaagttaggttgttctgttactggcgtatcatcgggtttagagtgtgttaatgtacttgggtcatccagctcagtttaccaaatagtactcgtggagcttgatatgcgtgagcttgacgggtttgaagttgcaacacggatctgtaaactcaggagcagaagtcgcagctggcctttgatcattgctttaacggcggctactgatgatgatcaattctgggacaagttcaaacaggttgggattagcggggttatcaaaaaacctgttcttttacaggagatgatcgatgaggtccgaagagtaattagtgcagccaaataa

SEQ ID NO: 73 - DNA, 2448 bp, Spinacia oleracea
  source 1..2448: mol_type = genomic DNA
atggaaatgcctgctagaagatcaaactacacacttttaagccaagtcgatgatcatacaactccgccgccgtcgacggtggcttcgaagttttccggtgttggaggaggagagagaagcttgtttccaccgtcgatcgggttgcagaggcagagcagtgggagtagttttggggagagttcattgtcggatcattacgtgccgtcgatctctttgccggcgagcgatctcgacggcttcagctacatgcaggaggagagagattcgaggaagagggtagcgattgatggtggcggcggcggtggaggatcttcggcgtcaaagagttgggcgcagcagaccgaggagagttatcagttacaattggctttggctttgaggctttcttctgaagctgcttgtgctgatgatcctaatttcttggatccgatgccagatgaatcttcttctcggttgtcttcctctgagtccgctgaatccatttcccatcgtttctgggtgaatggctgcctctcgtactctgacacaattcctgatggattttatctcattcatgggatggatccttatgtttggacagtttgtgctgatttacaagagagtggtcgaattccttctattgaatccctaaagctggtaaattctgtaaatgaatcttcacttgaggtagtattgattgatcggcgtggtgattccagtttgaaggacttgctgaaccgagctctggatatatcctgtaaatgcgtgacctccgatgatgttgttgatcagctagcaaagctggtatctagccgcatggggggcgtcgcttcaaccagagaggaggaattggttcccatctgggaggagttcagtaatggtctcaaggattgtctgggttctgttgtgcttcctgttggtagcttgtctattggcctatgcaaacatcgggctttgctattcaaagtccttgctgattcaattgatttgccgtgtcgaattgccaagggatgtaaatattgcaatagaaatgatgctgcctcttgcttagtacggtttggacttgacagggagtatttggttgatttggttggaaagccaggttgcttatgcgagcctgactccttgcttaatggcccatcatccatatcaatttcttcaccactacgctttccacgatttagatcagctgaacctgccattgactttagatcactggccaaacagtatttttcagattgtcaatctctgaatcttgtatttgacagaccttctacaggtgcagttgtgaatgagcaagatgcaggagatcctatgtatcttaagcaacttggtagaaagctgcccgacgtaaacaatcctataccaaatgaccaagtacattctcagtttgttggtcatgatagagaaccccagtgttctgaatctagtaatcctccacaaaatattggaagagcatctaatgccatcagagagtttcttccatcaagatttgttcctcctagcagacgagggacccaggcattaactgattcagcgagaaatgcatcaaaagattttcggtttgaggctagtcaactagtttcaagtagaagtgaagctgcactggacatggacgatttggtcatctcgtggagcgatcttgtcgtaagggataggattggagcaggttcctttggaactgtccatcgtgctgaatggaacggatcggaggttgcagtcaaaattctcatggaatcagatttccatccagagcgctgtaatgaattcttgagggaggtggcgataatgaaacgcttacggcatccaaatatagttctcttcatgggggcagttactgaaccacctaacttggccatagtgacagaatatttatgcagaggaagtttatacaggcttttacataaatcgggtggcagagaagtattggatgagaggcggcgtctgagtatggcatatgatgtggcaaagggaatgaattatcttcataagcgtaatccacctattgttcatcgggatcttaagtctcctaacctcttagtggataagaaatacactgtgaaggtctgtgattttggcctttcgcgcctgaaggcaaatacttttttgtcgtcgaaatctgctgctggaacacctgagtggatggcaccagaagttctgcgtgatgagccatcaaatgagaagtcagatgtctacagctttggtataatattgtgggagcttgcaacattgcagcaaccttggaacagcttaaatccagctcaggttgttgcagccgttggtttcaagggaaagagacttgatattcccagtgatgtaaatccacaaatagctactattatcaaggcgtgttgggcaaatgagccttggaggcgtccatcctttgccaaccttatggattccttgaggtcacttcttaaacctcctgcacctcaatcagttcgtggagatgcacacattattacgtaa

SEQ ID NO: 74 - DNA, 1146 bp, Spinacia oleracea
  source 1..1146: mol_type = genomic DNA
atggattgtttcgaagcattgcactcaacaggaggaggagatggcaaccaagaatccaaaattaaaggagtgatgacacacgggggtcgttatgttcagtacaatgtccatggcagcttatttgaggtttcttctaagtatgttcctcctattcgtcccattggccgtggcgcttctggccttgtttgtgctgctgttaattcagaaacacaccatgaaattgctatcaagaagattggtaatgcattcgacaatatagtagatgcaaaaaggacactgagagagatcaaactacttcgccatatggatcatgaaaatattattgcgattaaggatatcattcgaccaccaaagagagaagcattcaatgatgtttacattgtttatgaactgatggatactgatcttcatcagataattcgctctaaccaagctttgactgatgatcattgccagtacttcttatatcagctcttacgtggcttaaaatatgtacactctgcaaaggtcttacatcgcgatctcaagcccagtaatttacttctaaacgcgaactgtgacctaaagattggagatttcgggctggctaggaccaccactgagacagattttatgactgagtatgttgtaactcgctggtatcgagctcctgagttgctccttaattgttcggaatacacggctgcaattgatgtctggtctgttggttgtatatttggtgaaatcatgacaagagaacccttgttcccaggcaaagattatgttcaccaactaaggcttattactgagttgatagggtcaccagatgattcaagcttaggatttctgcgaagtaaccatgcgaggaggtacgtgaaacagctccctcagtaccccaagcagcagttctcagcaaggttccctaacatgtctccagcagccactgatctgttagagaagatgcttgtctttgatcccaacaaacgcattaccgtggacgaggcactgtgtcacccacacttagcctctctccatgacattaatgatgaacccatctgctcagcacctttccgttttgatttcgagcaaacatccttgacagaagagaatgtcaaagagcttatctggaaggaatcagtcaagtttaacccagatccagctcattaa

SEQ ID NO: 75 - DNA, 1116 bp, Spinacia oleracea
  source 1..1116: mol_type = genomic DNA
atggagaactcagcaaaatccaaggaagttcgaggagttcctagctatggcggaaggtacatgatgtacaatgttctcggcaatctttttgaggtttcctccaaatacgcacctcctattcaacctgtcggtcgcggtgcttacggcattgtttgctgtgcacgaaattcagagacacaagaagaggtcgccattaagaagattgcagatgcttttaacaatagaattgatgctaaaaggactttgcgagaaatcaaacttctgtgtcacatggaccatgacaatgttatcaagataaaggacatcatacgaccacctgaaagagagaagttcaatgatgtctacattgtttatgaattaatggatactgatcttcatcaaattactcgctctaaccaggaactcacagaagatcattgccagtacttcttatatcaaatcctacgcgggctaaaatatatacactcggcaaatgttctgcaccgggatctgaaaccaagcaatttgcttttaaatgctaattgtgacctcaagatatgtgactttgggctcgcaagaaccactgcagagacggattttatgacagagtatgttgtgactagatggtatcgagctccggagttgcttcttaattgttcagagtatacaggagcgattgatatgtggtcagttggttgcatcatgatggagattataaaaagggaacccctttttcctggaaaagactacttgcagcagttacagctcataaatgagctgctaggctctcctgaagatgctgatcttggatttttgagaagtgaacatgctaaaaagtatgtaaaacagttgccacgtgtcccaaagcagccattttcgcagaaatttccaaacgtgtctcccttggtgcttgatcttgctgaaaagatgcttgtattcgatcccagcaagcgaataacagttgatgaagcattgaatcacccatatttgtcgagcctccatgagataaacgaggagcctatctgcgcaaaaccttttcagtttgattttgagcaatcatccttggatgaagaagatataaaggatcttatatggcgggaggccctaaagttcaatccagataagatgattgaataa

SEQ ID NO: 76 - DNA, 1209 bp, Spinacia oleracea
  source 1..1209: mol_type = genomic DNA
atggaaaaagtttccacagaaattcacgcgtcgtcaatgaatttcccacgcgacaaagaaagaaaaaaagaaaaagaaaaagaaaatacggaaaaaccccatcaaaaaatggagcagaatttgagaggagtacccactcatggaggaaggtatgtacagtacaatctgtacggtaaccttttcgaagtttctagtaagtatgtccctcctcttcgccccgttggccgtggcgcgtatggtattgtctgtgctgctgtcaattcagagacacgagaagaagttgcaatcaagaaggttagcaatgcatttcataacagaattgatgccaaaagaatactccgcgaaatcaaacttcttcgccacatgaatcatgacaatgttatttccatcagggacctaatacgacctcctcagaaagagaacttcaatgatgtatacatggttcatgaactcatggatactgatcttcaccaaataataagatccaaccagcagttgacagatgatcattgccggtattttctgtatcaaatattacgagggttgaagtacattcactctgccggtgtgctgcaccgtgatctgaaacccagcaatttactaatgaatgcaaactgtgaccttaagattggagactttggacttgccagaacaacttctgaagccgatttcatgactgagtatgttgtaactcgatggtatcgagcaccagaattacttctaaattgttcagaatacactgctgcaatcgatatttggtctgttggatgcatacttggggaaatcatgactagagaaccccttttccccggcaaggactatgtcaatcagttgaaacttattaccgagctgatagggtcacctgatgactcgagtatagggttcctacagagtgacaatgctcgaagatatgttaagcagctaccacaatatccaaggcaacaattctctgctaaatttccaaatgcttctccgttggctattgatatgctggagaaaatgctcgtctttgatcccagcaagcgtgtaacagtgaacgaggcactcagccatccatatcttgcccctctacacgacataaacgaggaaccagtttgtcctcagccattcagttttgattttgagcagccgtctttcacagaggaaaacatcaaggagctcatttggagggagtctgtcatattcaacccagatcaccccattgcttga

SEQ ID NO: 77 - DNA, 3033 bp, Spinacia oleracea
  source 1..3033: mol_type = genomic DNA
atgcgaatcctccttcttcttcttcttcttctcttgcttcttcttgatttcaataatggcagaacattaccagaacaaaatgctttactaggcattaaatccgccattacagacgacccacaagcttctctttcttcctggaacaccaccacccaccactgcacttggccttttgtcatttgctcaaccactgttaaccaccattctgccgtcgtctctctaaacctctcgggtttaaacctcaccggaatcctctcccctgatattggctttctcactaacctacaaaaccttactgttgcgtctaactccttttctggccctattccgccgtctgtctctcttctcaccgagctccggtacctcaacctctcaaacaacatcttcaatgaaaccttcccttcttctctctcctcactcaagaagctaaatatccttgatgtttataacaacaacctcactggaccgcttccgctgtctgccgttggtatgacggagctccggcatcttcaccttgggggtaactacttttccggcgagattccaccggagtacgggcaatggaagaagctagagtaccttgcggtatccgggaacgagcttgaggggaagattccgggggagatcggtaacttgacttccctcaaagagctttacattgggtactttaatggctacaccagtgggattccgccggagattggaaacttgacggagttagtccgatttgacggtgctaattgtggattatctggtgaactaccggcggagttggggaagcttcagaagctggatacgttgtttcttcaggtgaacactctttctgggtcactcccagctgagttgggaaccttgaaaagcttgaaatccatggatttatccaacaatttgttctccggtgagattcctgaatctttcgctctcctaaaaaacctaaccctgttaaacttatttcgaaacaaattgcacggtgccgtgccggagttcataggagagttgcctgatttagaagttcttcagttatgggagaataattttacaggaagtatcccggaaaagttgggtacaaatgggaaattgcagcttctagatctttcatctaacaagcttacaggtagtttacccccaaatctttgcagaggagagagattacaaaccctaattacactgggaaatttcttacttggcccaattcctgagagtttgggtagttgcagctcacttgcacgggttagaatgggggagaattacttaaatgggtcaattcctaaaggattgtttggtctacctaaattaacccaagttgagttgcaggataatctccttaccggtggtttccctgaggcaggaaacactgtggcggcgagtctcggtcagatttcattgtcgaataatcagctgaccgggaggttaccggcaagtatcggtaacttctctggtgttcagaagttgttgctagatgggaacaggtttagtgggagtatcccagctgagattgggaggttacaacagctttctaagattgattttagtaataacaagttttcgggtgagataccgccagactttagtaagtgtaagctgcttactttcgtcgatctcagtcggaatcagctcggtggagagattccgaaggagattacaggtatgaggatattgaactacttgaacatatcaaggaatcatttgagtggtagtattcctggatcaatttctagtatgcagagtttaacatcggttgatttttcatacaataatttcactggtttggtgcctgaaactgggcagtttagttactttaattacacttcctttttgggtaatcctgatttgtgtggcccttatttgggtccttgcaaaaatggggttggtggttcttcacaccaaaatcatggtagaggcctatctgcttcattgaagctattcttggtcattggccttctgttgtgttccataatctttgcaattgctgccataatcaaggctcgttcgcttaagaaagcaagcgagtctcgagcatggaagctgaccgccttccaacgattagatttcactgcggatgatgtgttggattgcttgaaggaagataacattattgggaaaggtggtgctgggattgtttacaaaggaaatatgccaaatggtgatcaagtcgctgttaagcgtcttcctgtaatgagtaggggttcatctcatgatcatgggtttaatgctgagattcagactttagggaagattagacataggcatattgttagattgttagggttttgctccaaccatgagactaatctgttagtgtatgagtacatgcctaatggaagcttaggggaggtgatacacggtaagaaaggcggtcatttgcattgggatacaaggtataagatcgctgttgaatcagccaaggggctttgctatctgcatcatgattgctcgccgttgattgttcatcgcgatgtcaagtccaacaacatccttctggattccagtttcgaggctcatgttgctgatttcggccttgctaagttcttgcaagattcaggcacttcagagtgcatgtctgctattgctggttcctatggatacattgccccagaatatgcctacacattgaaggttgatgagaaaagtgacgtttacagctttggagtggttctgttggaactgatcactggtaggaagccagttggtgaatttggtgacggggttgatatagttcaatgggtaagaaaaatgactgatggcaacagggaaggagtcctaaaagtgttagaccctagactcccttcagttccactacatgaagtaatgcatgtattctatgtggccatgttgtgtgttgaagaacaagccgtggagcgacccactatgcgagaggtggttcaaatactacttgagatcccaaaaccaccagcatctgcaaagcaaggtgacgactcacctcaacaaactactacagatgccacaagttctccaacaaacgaaaaccagccaccaccaaaatcaccaccgcaagatcttcttagtatttag

SEQ ID NO: 78 - DNA, 2943 bp, Spinacia oleracea
  source 1..2943: mol_type = genomic DNA
atggcgacggaaggaggtggtggaggggcggcggcagcggcgaggaggaagccgtcgtggagagaaagagagaataatcggaggagagagagaaggaggagagccatagctgcgaagatattttccggtctccgagctcaaggaaactataatctccctaaacattgtgataacaatgaggttctgaaggctctttgttctgaagctggctggattgttgaacctgatggtactacttaccgcaagggaatgaggccgccaccgccggagattgtggctagttttaatcattttagtgcgtgttcatctcaagtgccgagtcctctatcgtcggcatttcagagtccaattcattcctaccaacctagtccgtcttcgtctgctccgcaaagcccgactcgaaacgagatcaatgcacaatcccatccgttctcgttcctctgcaattctattccgtcgagtctgccacctcttcggatatcgaatagtgccccggtgactcctctgttatcgtctccaacgtcaaaaactgctcctaagcagccagtttttaatttcgaggccattgctaatgagtctatggcatcttttaattatcctatgtttgctgcttctgctccttccagtccttcaagacggaggaactttaccccgcctaccatacctgaatgtgatgaatctgactgctctactattgattccggacagtggattagcttccaaaggttttccagacctccaattccaaattcccccacatttaacttggttaggccaatggttcagaaaatgtccttgaatgatgcaattcaggatagagcaagagttgctgaagctgagagtatgcaggttaccccttgggagggggaaaggattcatgatgtaggcatggatgatcttgaactcactcttgggagtggtactgccaggaactaaatggattattcgacttcatggttgagccactcatcaccttctctcgatatttctaatgctaacacacttcgccttttcgacgatgcccctgttagagataccaacatcttcaagcaagatccttccccacctcgccctccttctaccaatctttggcacaaagatgagagtggagcattagtagaggaattgaagagggttaatgcagagaacaagagactaacagaaatgctaaccgtagtttgtgaaaactacaatgaattgaagagacagttggtggatcatatgagcaagcagagttcaagtaataatgagattaccaagaaaaggaaactggacaacaacaacaacaacaacaacaacaacaacaataataataacaatggccttgttgttagaactaataataataataatggtgagagtagttccagtgatgaagaggattcctatggaaaacctaaagaagagactattaaagctaagatcacaagggttgctgttcgtactcaagcttctgattccacccttattcttaaggatggataccaatggagaaaatatggccaaaaagttacaagagataacccttgccctagagcttacttcaaatgctcctttgctcctggttgccccgttaagaaaaaggtacaaagaagtctagaagaccagtcaatgttagtggcaacgtatgaaggagagcacaaccaccaaccaccagcacaacaagagggtcagggtggtgcaccatcgggttctagccggtccttgccccttggctcagtcccatgcactcttgacctaaccaagtccaagtccaatgctaacaacgtcgtaaacgaaacaacaactacaaccactagcagcaacaacaacaacaacaacaacaacaacaacaacaaggtgttgaataacacgacgtcgtctaaccataaccagttgccaaagtttagctcaccagaattccaaaaactattggcagagcaaatggcttcatcgttgactaaagatcccaacttcacagcagcgttggcagcagccattaccgggagattcgtccagcaaacttcaagtgattaaatggagtcatcatctaatttggttcttgacacttctcttgggttcaatcttaaccttaatctttgtcacaatccggaaatcttcgatgacaaaacacaggtggacgagagaaaaagtgggaatttagaagaggagttgaacaggatgaacatagagaataagaagctgacaaaaatgttggcatccatgtgtaagaattacagtatgctgcaaaatcagttgattggtttgattagtccgacgtcgaaaggcgaaaatgaagatcagaatccaaggaagaggaagtttattgaagctgtggagtgtggaatacaggggaacacagaacagagttgtattagctacgatgattcattcaaaagagtaactcttcttagcagcaagcctaagatcactaataaaatgttgattcggaccgataaatcggatattagcttggtggtgaaagatggatatcaatggaggaaatatgggcagaaagtgacgagggataatccatctcctagagcttactataaatgtgctaatgctcctgaatgtcaagtcaaaaagaaggtgcaaagaagtgcagatgatccatccatcttggtagcaacatatgaaggagaacacaatcatcctcaaccaaattcaagggatcatttagatcaacaagtggtattaagctcaagtcaatcagtaaataataatactaataatgttataagatctcctaatacattaaaccttgatatgatcaaccataattatcccaataacatggagaagacggtcgactacaacgacgtcgacacgaaagcgttgcagagattacttgtagaacaaatggcttcttcattgacaaaagatccttcttttacttctgcattagctgctgccatttctggaaaaatatcgaacaatgcttatactcagagatggttataa

SEQ ID NO: 79 - DNA, 1791 bp, Spinacia oleracea
  source 1..1791: mol_type = genomic DNA
atgtctagctcgtcggtgattggtgattccggtagatttcccggccaaaagatgagtttctcgcagacttgtaaccttctcagccagtatgttaaggagaaacgcgctcaaggtgttactggaaacatcgagtcccgaggaccaacttcaacgatgaatttgttccctgtgagtgggaaacatgcagctgaagattcaaatgtttgtgctgggaaaacaatcaatctctttcctcagctttctgggtttaattcaagcgtgaacaagcgtgctaaccccgagcctccgactccccaaatgaccatcttctatggagggcaagtcttggtctttaatgacttgccgactgacaaggcaaaagaagtcatggacctagcttgtagctttgaagctagcctcaagaagaggaaagtcgaacctaccagttcgagtccccctagcccggttgcagcccctgttcctgcccaaaaccccaaaccaaccctgaatcagaccctgattccaacccagaaatcaaaccagaacttgactgttgctccaaaatttgtgaagaacgtcataccagctaccgtgcctaatgtttctgatctgcctattgctaggaaggcctcactccaccgtttcctcgagaagagaaaagataggcttgtgggcaaactccccgcggaaattccaaatggaagcccggcaaagcccctagagggtaaggcagcgtggctcggactgaattctgtgccagtgtttcagatgcagaagccgcagcagcaatcctagatggattgtaatcttgagcttggcctatcaccatcttccacgtcgatctcttcaacagcttctacgatctccatgggatctgttaatggagataatccaaaaaagcaagaacaacaaaggacgatattttataatggaactatctgcgtttgcgatgttaccgatgttcaggcaaaaagcataatacagttagcaagtagaaaaacgatacaagaaatatgtacatctacaacaataacaccaacgttacaatctcaacaattatgtactcaaacgcaaaatggtttatcggccatgaagaaatcgttacgccaatttctcgaaaagcgaaagcatcgaatccaagctacctcgccttaccgtctttagatgtctaaatcgtctcacgttgaactcgatttctttggtttgcacaaccttgccaattccatggctcatcctcctcagcctcagacacaaccgcagccgcagccgcagccacagcctcgccttgatcgccgtcaaagtttccgtggaattgcaaagataaatccggagatagtgaagagtgtgattgcatcagctaatgcttccaagtcctcatcatcacatcccgggactcccacttcaagtcttggaatgtctcaacttcccctctttcatcccaatcctttttctggggaagatagcatggagaaagcagaagaaactgccccaatgaccatattctacaatgggtcagtttctgtttatgatctacccaacgacaaggccgaaagcataatgaagcttgcaatggaaaagaatgaatcaaaaaacgaagggaaggtggtggattattcatgttttaagtcatcccaagcaactactaagaatatcctcggacgacttaatgatgatgatggagatgattctgatatgcctattaaacgtaagaagtcattgcagaggttctttgagaagcgcaaagagaggttggtgtcgccatatacaaacatagaacacatggcgtctggaatagagcagcaaaaggcctaa

SEQ ID NO: 80 - DNA, 2079 bp, Spinacia oleracea
  source 1..2079: mol_type = genomic DNA
atgtctagctcgtcggtgattggtgattccggtagatttcccggccaaaagatgagtttctcgcagacttgtaaccttctcagccagtatgttaaggagaaacgcgctcaaggtgttactggaaacatcgagtcccgaggaccaacttcaacgatgaatttgttccctgtgagtgggaaacatgcagctgaagattcaaatgtttgtgctgggaaaacaatcaatctctttcctcagctttctgggtttaattcaagcgtgaacaagcgtgctaaccccgagcctccgactccccaaatgaccatcttctatggagggcaagtcttggtctttaatgacttgccgactgacaaggcaaaagaagtcatggacctagcttgtagctttgaagctagcctcaagaagaggaaagtcgaacctaccagttcgagtccccctagcccggttgcagcccctgttcctgcccaaaaccccaaaccaaccctgaatcagaccctgattccaacccagaaatcaaaccagaacttgactgttgctccaaaatttgtgaagaacgtcataccagctaccgtgcctaatgtttctgatctgcctattgctaggaaggcctcactccaccgtttcctcgagaagagaaaagataggcttgtgggcaaactccccgcggaaattccaaatggaagcccggcaaagcccctagagggtaaggcagcgtggctcggactgaattctgtgccagtgtttcagatgcagaagccgcagcagcaatcctagatggattgtaatcttgagcttggcctatcaccatcttccacgtcgatctcttcaacagcttctacgatctccatgggatctgttaatggagataatccaaaaaagcaagaacaacaaaggacgatattttataatggaactatctgcgtttgcgatgttaccgatgttcaggcaaaaagcataatacagttagcaagtagaaaaacgatacaagaaatatgtacatctacaacaataacaccaacgttacaatctcaacaattatgtactcaaacgcaaaatggtttatcggccatgaagaaatcgttacgccaatttctcgaaaagcgaaagcatcgaatccaagctacctcgccttaccgtctttagatgtctaaatcgtctcacgttgaactcgatttctttggtttgcacaaccttgccaattccatggctcatcctcctcagcctcagacacaaccgcagccgcagccgcagccacagcctcgccttgatcgccgtcaaagtttccgtggaattgcaaagataaatccggagatagtgaagagtgtgattgcatcagctaatgcttccaagtcctcatcatcacatcccgggactcccacttcaagtcttggaatgtctcaacttcccctctttcatcccaatcctttttctggggaagatagcatggagaaagcagaagaaactgccccaatgaccatattctacaatgggtcagtttctgtttatgatctacccaacgacaaggccgaaagcataatgaagcttgcaatggaaaagaatgaatcaaaaaacgaagggaaggtggtggattattcatgttttaagtcatcccaagcaactactaagaatatcctcggacgacttaatgatgatgatggagatgattctgatatgcctattaaacgtaagaagtcattgcagaggttctttgagaagcgcaaagagaggttggtgtcgccatatacaaacatagaacacatggcgtctggaatagagcagcaaaaggcctaaatgtttcagatgaggttacaattccgatcatatgatcctatccaacctatctcattcgttctactacaaaggaatttcaggtgtagaaatatgggtgatagtgatattttggcgtcggttcgctctgtggtgttcaaggagtcagaaaccctagaagggaaatgcattaagactgagagttatacccagctttttaaatcgtttgtctccaccggtttccaagcatcgaatctcggagaagctattgatgttgttaatgaaatgattgacgactggaaaatgagatag

SEQ ID NO: 81 - DNA, 1446 bp, Spinacia oleracea
  source 1..1446: mol_type = genomic DNA
atgtcggacgaagaacaccacttcgaatccaaggctgatgccggagcttctaagactttcccccagcaagctggtactatccgcaagaacggttacattaacatcaagaatcgtccttgcaaggttgttgaagtttctacttctaagactggcaagcacggacatgctaaatgtaactttgtcggtatcgacatcttcactgggaagaagcttgaggatattgttccatcttctcacaattgtgatgttccccatgtcacccgtactgattaccagctccttgacattactgaggatggttttgttagcctgctgactgataatggagatactaaggatgacttgaagctgccaactgatgaagttctgaataaacagatcaaggatggatttgctgaggggaaagacctgattgtgagcgtgatgagtgccatgggagaagaacaaattaatgccctcaaggacatcggacctaagtaaatgtctgacgaagaacaccagttcgaatcgaaggctgacgctggcgcctcaaagacctacccacagcaagctggtaccatccgtaagaacggttatatcgtcatcaagaatcgcccttgcaaggttgttgaagtttccacttctaaaactggcaagcacggtcatgctaagtgtaactttgttggtattgacatcttcaatgggaagaagcttgaagatattgttccatcttctcacaattgtgatgttccccacgtcaatcgtaccgattatcagctcattgacatagctgaggatggttttgttagcctgctgactgagagtggaggtacaaaggatgacttgaagctgccaactgatgaaactcttcttaagcagctcaaggaaggatttgcggatggaaaagacctgattgtgagtgttcagaatgccatgggagaagaacaaatttgtggcgtcaaggatattggtggtggatctaagtagatgtctgacgaagagcaccagttcgaatccaaggctgacgccggtgcgtccaagactttccctcagcaagctggtactatccgtaagaacggttacatcgtcatcaagaatcgcccctgcaaggttgttgaagtttccacttctaagaccggcaagcacggccatgccaaatgtaactttgttggtattgatatcttcaatgggaagaagctcgaggatattgttccatcttctcacaattctgatgttccccatgtcactcgtactgattaccagctccttgacataactgaggatggctttgttagcctgctgactgacaatggagacactaaggatgacttgaggctgccaactgatgatgctctgctgaaacagcttaaggacgggtttgccgaggggaaagacctgattgtgagtgtgcagtgtgccatgggagaggaacagatctgcggtctcaaggatattggacccaagtag

SEQ ID NO: 82 - DNA, 3471 bp, Spinacia oleracea
  source 1..3471: mol_type = genomic DNA
atggattgtttcgaagcattgcactcaacaggaggaggagatggcaaccaagaatccaaaattaaaggagtgatgacacacgggggtcgttatgttcagtacaatgtccatggcagcttatttgaggtttcttctaagtatgttcctcctattcgtcccattggccgtggcgcttctggccttgtttgtgctgctgttaattcagaaacacaccatgaaattgctatcaagaagattggtaatgcattcgacaatatagtagatgcaaaaaggacactgagagagatcaaactacttcgccatatggatcatgaaaatattattgcgattaaggatatcattcgaccaccaaagagagaagcattcaatgatgtttacattgtttatgaactgatggatactgatcttcatcagataattcgctctaaccaagctttgactgatgatcattgccagtacttcttatatcagctcttacgtggcttaaaatatgtacactctgcaaaggtcttacatcgcgatctcaagcccagtaatttacttctaaacgcgaactgtgacctaaagattggagatttcgggctggctaggaccaccactgagacagattttatgactgagtatgttgtaactcgctggtatcgagctcctgagttgctccttaattgttcggaatacacggctgcaattgatgtctggtctgttggttgtatatttggtgaaatcatgacaagagaacccttgttcccaggcaaagattatgttcaccaactaaggcttattactgagttgatagggtcaccagatgattcaagcttaggatttctgcgaagtaaccatgcgaggaggtacgtgaaacagctccctcagtaccccaagcagcagttctcagcaaggttccctaacatgtctccagcagccactgatctgttagagaagatgcttgtctttgatcccaacaaacgcattaccgtggacgaggcactgtgtcacccacacttagcctctctccatgacattaatgatgaacccatctgctcagcacctttccgttttgatttcgagcaaacatccttgacagaagagaatgtcaaagagcttatctggaaggaatcagtcaagtttaacccagatccagctcattaaatggagaactcagcaaaatccaaggaagttcgaggagttcctagctatggcggaaggtacatgatgtacaatgttctcggcaatctttttgaggtttcctccaaatacgcacctcctattcaacctgtcggtcgcggtgcttacggcattgtttgctgtgcacgaaattcagagacacaagaagaggtcgccattaagaagattgcagatgcttttaacaatagaattgatgctaaaaggactttgcgagaaatcaaacttctgtgtcacatggaccatgacaatgttatcaagataaaggacatcatacgaccacctgaaagagagaagttcaatgatgtctacattgtttatgaattaatggatactgatcttcatcaaattactcgctctaaccaggaactcacagaagatcattgccagtacttcttatatcaaatcctacgcgggctaaaatatatacactcggcaaatgttctgcaccgggatctgaaaccaagcaatttgcttttaaatgctaattgtgacctcaagatatgtgactttgggctcgcaagaaccactgcagagacggattttatgacagagtatgttgtgactagatggtatcgagctccggagttgcttcttaattgttcagagtatacaggagcgattgatatgtggtcagttggttgcatcatgatggagattataaaaagggaacccctttttcctggaaaagactacttgcagcagttacagctcataaatgagctgctaggctctcctgaagatgctgatcttggatttttgagaagtgaacatgctaaaaagtatgtaaaacagttgccacgtgtcccaaagcagccattttcgcagaaatttccaaacgtgtctcccttggtgcttgatcttgctgaaaagatgcttgtattcgatcccagcaagcgaataacagttgatgaagcattgaatcacccatatttgtcgagcctccatgagataaacgaggagcctatctgcgcaaaaccttttcagtttgattttgagcaatcatccttggatgaagaagatataaaggatcttatatggcgggaggccctaaagttcaatccagataagatgattgaataaatggaaaaagtttccacagaaattcacgcgtcgtcaatgaatttcccacgcgacaaagaaagaaaaaaagaaaaagaaaaagaaaatacggaaaaaccccatcaaaaaatggagcagaatttgagaggagtacccactcatggaggaaggtatgtacagtacaatctgtacggtaaccttttcgaagtttctagtaagtatgtccctcctcttcgccccgttggccgtggcgcgtatggtattgtctgtgctgctgtcaattcagagacacgagaagaagttgcaatcaagaaggttagcaatgcatttcataacagaattgatgccaaaagaatactccgcgaaatcaaacttcttcgccacatgaatcatgacaatgttatttccatcagggacctaatacgacctcctcagaaagagaacttcaatgatgtatacatggttcatgaactcatggatactgatcttcaccaaataataagatccaaccagcagttgacagatgatcattgccggtattttctgtatcaaatattacgagggttgaagtacattcactctgccggtgtgctgcaccgtgatctgaaacccagcaatttactaatgaatgcaaactgtgaccttaagattggagactttggacttgccagaacaacttctgaagccgatttcatgactgagtatgttgtaactcgatggtatcgagcaccagaattacttctaaattgttcagaatacactgctgcaatcgatatttggtctgttggatgcatacttggggaaatcatgactagagaaccccttttccccggcaaggactatgtcaatcagttgaaacttattaccgagctgatagggtcacctgatgactcgagtatagggttcctacagagtgacaatgctcgaagatatgttaagcagctaccacaatatccaaggcaacaattctctgctaaatttccaaatgcttctccgttggctattgatatgctggagaaaatgctcgtctttgatcccagcaagcgtgtaacagtgaacgaggcactcagccatccatatcttgcccctctacacgacataaacgaggaaccagtttgtcctcagccattcagttttgattttgagcagccgtctttcacagaggaaaacatcaaggagctcatttggagggagtctgtcatattcaacccagatcaccccattgcttga

SEQ ID NO: 83 - DNA, 6756 bp, Spinacia oleracea
  source 1..6756: mol_type = genomic DNA
atggagtcgtgcaattgtattgagccgcaatggcctcatgatgatctgttgatgaagtatcaatatatttctgattttttcatagcccttgcttatttttcaatcccattggaattaatttactttgttaaaaaatcagctgtttttccgtatagatgggttcttgtgcagtttggtgcttttatagtgctttgtggagcaacacatctaatcaatttatggacatttagaatacatactaaaacagttgaagtagttatgactaccgctaaggttttaaccgctgcagtgtcgtgtgcaactgcactgatgctcgtgcatataattccagatttattgagtgtgaaaactagggaattgtttttgaaaaaaaaggctgcagatctggaccgagagatgggcctcatccgtacccaagaagaaactgggagacatgttaggatgctgactcatgagatcaggagtactcttgaccgacatacaatccttaaaaccactcttgttgagcttggtaggacattggctcttgaagagtgtgctctatggatgccaacgcgtacaggattggaacttcaattagcttatacactccgtcaacaaaatcctgttggatttaccgttcccatacaacatccagtaatcaatcaagtattcagttccaatcatgctgtgcaaatctcaccaaactgtcctgtagctagaatccgtcctatctctacgaagtacatgcctggagaggtggttgctgtgcgtgttccccttcttcatctttctaatttccaaatcaatgattggccagagttatcgacaaaacgttatgcgctgatggttttgatgcttccttcggacagtgctagacaatggcatgttcatgaattggagcttgttgaagttgttgcagatcaggtagcagttgccttgtcccatgcggcaattttggaagaatctatgagagctcaacaacaacttttggagcaaaatgttgcacttgatttggctcgcagagaagcggagactgccatccgtgctcgaaatgactttttggctgtgatgaatcatgaaatgcggacacctatgcatgcaattatagccctttcctccttactgcaagaaactgagttgatccctgagcagcgattgatggttgaggcaattctaaaaagcagtaatcttcttgcgactctgataaatgatgttttggacctttcacggcttgaagatggaagtttgaagcttgataatggagtatttaatctccacacgatgtttagagaggtccttaatttgatcaagcccatagcatctatcaaaaagttgttgataacgacgaatttggcaacggatgtgccagagtttgctgttggtgatgaaaaacgtctaatgcaggttctattgaatgttattggtaatgctgtgaagtttacgaaagaaggaagtgtctcagttactgctttagttgcaaaagcagaatctttaagagatgctcggactcctgatttcattccattagcgacagatgatcacttctatttgcgagtgcaggtgaaggattcaggacctggaatcaatccgcaagatattccaaagctcttcacaaaatttgcccaaccacaaacacctgccaatcgaaattctggtggtagtggactgggccttgccatctgcaagaggtttgtaaatctcatggaagggaatatctggattgaaagtgaaggtcttgggaagggttgcacagctatttttgtagttaaacttggaagtcccgagcgttcaaatggagccatggtcccttttgcatccaaaccattggtgaatcatattcaatcgagctttggtggcctcaaagttctcattatggatgagaatggggttagcagaacagtgacaaaaggacttctgatgcacgttggatgtgatgtcttgactgtagcgtctagtgaggaatgtttgttagctgtctcccgagaatacagggtggtattccttgatttgtctatgcctggatttgatggctttgaagttgctgttcaaataagaaaaaggttctcaaagcgtcatgaaaggccgttaattgtagcactcactgggagtgcggacaggttgacccgagaaaattgtgtcagggtgggtatggatggttgcgtattgaaacctatatctgttgataaaatgagaagtgttttatctgagctcctagaaagacgaactgtgtttgagccgctctagatgatgagactgatagccattggattgttggtttgcactctcatattatcttcatctaccattggttgtggatattcccattgtaattgtgaggatgaagggttttggagcattaatatcattatggaatgccaaaaagtgagtgatttcttaattgccattgcttatttttcgatacccatcgagctcgtgtattttattagctgtgcaaatattcccttcaaatgggtccttgtcgaattcattgctttcattgttctgtgtggaatgacccacttgctcaatggatggacttactatggtcctcactcatttcaactcatgatgtcacttacaatatttaaattcctaacagctttggtgtcatgtgccacggctataacccttttaactttgatccctcttttacttaaactgaaagttagagaggtgtttttgacgcaaaatgtcttggaattggaccaagaggttgggatgatgaagaaaaagaaggaagctagttggcatgttcgaatgctcactcgagaaataagaaaatcactcgataagcttactatattggatactactttggttgaactttccaatatattagacttgcagaattgtgctgtttggatgccaaataatattaagactgcaatgaacttgacccatgagctgaagcctagcccctcaaatatctttccccgaaatatcccaatggatgatcatgatgttcaggagattcgtgatagtgagggagtgaggattttaagaccggaatcagcactgggagtggcaagcaggggtggttcggaagattctggtactgtagcagccattcggatgcctttgctgaatgtttcaaattttaaaggtgggactcctgaattagtacagacatcttatgccatactagttttggttcttccaagctcaactgatagggtttggagccatcatgaactagagatggtggaagtggttgctgatcaagtggctgtggccctttctcatgctaatgttcttgaagagtctcagttgatgagagatcagctgaaggagcggaatcgtgttctgcagcaggctaggaaggacgcgatcatggcaaaccaagcaagagattcattccagaaggttatgagccatggaatgaggaggccaatgcactcgattctcggtcttctttcaatgtttgaagagaatatgaattttgagcagaaaattatggttgacaccatggtgagaactggtaatgtcatctcaactttattaaatgatgtgacagaaatatctgcaaaagacaatggaaggttccctttggagatgaggctgttccacttgcattccatgattaaggagacttcttgcttggccaagtgtttatttgtgtatcaagggttcagtttttctcctgatgttcagagtagtttgcctgctcaggtgattggtgatgagaggagaacttttcaggttttactacacatggtagggcacttactgcatttttcaaacaaagggggtttgatgatttttcgtgtgtctgccgaaaatggcagtgtgggaaagaatgataagtgggggatgtggaggtcggctactgtcgaggagtacattaaagtaaagtttgagtttgacataagtggaggttatctgacagacactattaggagaacccagaatgattcaaacaaggaagtattgagcttcagcatgtgcaaaaggcttgctcagatgatgcagggtgatatatggatatctctaaactcaaatgggataccacagaacatgactcttgttctacgatttcaacaacaaagatatattcctgaaggaaacttatatatgacagagcacacagtttcttcaaactctcggttcagagggttgaaagtgttgattgctgatgatgataatgtaaatcggacagtgacaaagagactgctagagaagttagggtgtgaagtatctgctagttgttctggttatgagtgcttaactgcattggctccctcacaaaacacttatagggttgtgctacttgaccttcagatgcctgaaatggatggctttgaagtcgcaaggagaatccggaaattatatagccggagtggtccgtctattatagcaataacagcaagtgcagaggataatatatgggagagatgtttacaaattgggatgaatggtctaataaggaaacctgtggtcctgcagggaataatcgacgagctccgaagggtcctcagatgaatgttgagaccactggctgctgctctcttcttttctgctctgttgatatcagcttctgcctctgacaatggctacccaagatgtaattgtgaggatgaagggttctggagtgttgagagcattctagaatgccaaagggtgagtgatttcttgattgctgttgcctatttttcgatccctattgagctgctttactttgttagctgttcaaatgttccttttaaatgggttctctttcaattcatagccttcattgttctttgtgggatgacccatttacttaatggttggacttatggtcctcacgcctttcaactcgccgtagctctcaccacttttaagtttttaaccgctctcgtctcttgtgctactgccattacccttatcaccctcatccctttgcttctcaaagtcaaggtcagagaattcatgttaaagaagaagacttgggatttgggtagggaggttgggatgattaagaaacaaagtgaagctggatggcatgttaggatgcttactcatgagattcgcaagtccttagataggcatacaatcttgcacacaactatggttgagctctctaaaaccttggatttgcttacttgtgctgtttggatgcccaatgagaataggactgagatcattttgactcatggtttaaaaggaaggaattttcagagtttcagcaactgcagagttccctatgctgatcctgatgtcagaaaaatcaaggagagtaatgaggttcagatactcagaccagattcagctcttggtgttgccagtagcggtgggttggaaaattatggtggtgtggctgcaatacgtatgccgatgctcaaggtctcaaatttcaaaggaggaacccctgagctgatgcaggcttgctatgctatattagtcttggttctcccttgtgggcaacagagatcttggaccaaccaagagcttgagattatcatggtggttgctgatcaagttgctgtggctatctctcatgctgccatcttagaggaatcacagctcatgagagatcaattggcagagcagaatcgcgcgctgcagcaggcacaacgaaacgcattgatggcaagtcaagcgaggaactcgtttcagagagttatgagccatgggatgagaaagccaatgcactcagtcttggggttgctttcagttctaaaagatgagaatttaagcagtgaacagaaacttattgttgacacaatgatgaagacagggaatgtcctttctcttttgatcaatgatgtcatggacgattcaccaaaagagagtggaagatttccatcagattttagatcatttgggctccgtgccttggtaactgaagctggttgctttgctaagtgcttgtgtgttcaaaaaggttttggctttagtattgaagttgacagatccttgcccgatcaagtattgggtgatgaaaaaagggtatttcaggtgatcttgcatatgattggtaacttggtggagaacaataccggtggtgggtctttgttttttcgggtctactctcaacatggaagtcagggaaggaatgatcaaagatgggcaacatggcgatcaggctcttctgatggatatgtttatatccgatttgaagttgagataaccaataatgctatcttctcagatgattcagtgttggcttcaaactacagtggtccgagatgcagcagtgagattgtggaccgggacctgagtttcagcgtttgccagaagctagtccagctgatgcaaggaaatatatgggtagttccaaatccacagggttttcctcaaagtatggcactggtactacgtttccagctacgtccatcaattggaataacaatctcagaatcaggagaaccatcatcagataactcctcctccaactttactttaaagggacttcaaatactattagcagatgatgatgacatcaacagggcagtgaccaaaaagttgctggagaagttaggttgttctgttactggcgtatcatcgggtttagagtgtgttaatgtacttgggtcatccagctcagtttaccaaatagtactcgtggagcttgatatgcgtgagcttgacgggtttgaagttgcaacacggatctgtaaactcaggagcagaagtcgcagctggcctttgatcattgctttaacggcggctactgatgatgatcaattctgggacaagttcaaacaggttgggattagcggggttatcaaaaaacctgttcttttacaggagatgatcgatgaggtccgaagagtaattagtgcagccaaataa

SEQ ID NO: 84 - DNA, 9705 bp, Spinacia oleracea
  source 1..9705: mol_type = genomic DNA
atgatggaggattcacaaagtggggtgaagcccacaaagcaagcaaatgaacaagtctgccagatctgtagtgacaacatcggcacaactgtagatggagagccgtttgttgcatgtgatgtttgttcctttcctgtttgtcgtgcttgttacgagtatgagcggaaagatggaacccaatcttgcccacaatgcaagacgagatacaagaggcagaaaggtagtccagcaattcatggagaaaaggtggaagattctgatgttgaggatgtggtcagtgacgtaaatgagcccttgggaagttcaatcctaaaggagaagcaacaggagagaatgcttggatggcatatgaatcatgggcaatctggtgagcttggcccccctacctatgataaagaggctcctattagtcatatccctcggcttgctaccggacgcacggtttctggagacctgtcagctgcatctcctggacgtttttcaatgccatcccctggtgccagcactggagctaatattagagtgtcaagggaattcgcatcgcctggatttggaaatgtagcttggaaagaaaggattgatggttggaagatgaagcaagagaagagtacaggtcctcctagtgttagtcatgcgccttcagaaggaaggtttgccaatgatattgatgccagcactgaaattgctatggacgatcctttactaaatgatgagacacggcagccgttatcaaggaaggttcccattccttcatctagaattaatccgtacaggatggttattgttctgcgacttgctgttcttggtatctttttgcactaccgtgtaacaaatccggtgcctaatgcctatgcattgtggctgatatccgtcatttgtgagatatggtttgcattttcatggattctggatcagtttcccaaatggcttcccatcaatcgcgagacatatcttgacaggctggccctcaggtatgatcgtgaaggggaaccatcacagctggccgctgttgatatatttgtcagtactgtggatccgttgaaggaacctccccttgtcactgccaatacagtgttatccattcttgcagttgactatcctgtcgacaaagtttcttgctatgtttctgatgatggagctgctatgctgacatttgaagctctttcagagacatcagaatttgcaagaaagtgggttcctttcacaaaaaaatataatattgagccacgagctccagaatggtacttctctcagaagattgactacctaaaagataaggttcaaactacgtttgtgaaagaccgaagggctatgaagagagagtatgaagagttcaaaatccgaattaatggccttgttgcaaaggcaacaaaggtcccagaggaaggatgggtaatgcaagatgggacaccttggcctggcaacaataccagagaccatccagggatgatccaggttttcttaggccaaagtggaggacttgacactgatggtaatgaacttccacgtttggtttacgtatctcgtgaaaaacgtcctgggtttacacatcacaaaaaagctggtgctatgaattctcttgttcgtgtatcagcggttctcacaaatgggcccttcatgttgaatcttgattgtgatcattacataaacaacagcaaggcgttaagagaagctatgtgttttatgatggatccaaaccttgggaaatattgttgctacgttcagtttccacagagatttgatggaattgataggaatgatcgatatgctaaccgtaacactgtcttctttgatattaatttgagaggtttagatggtatccaaggacctgtctatgtgggtacgggatgtgtcttcaacagaactgctttgtatggttatgagcctcctatcaaaccaaagcccaagaagaaaggtatcctatcatcatgctttggtggatctcggaagaaatcaagcaagaaagactccaagaagaaatcaaaacatgctgaccctactgtgccaatatttaatctggaagatatcgaggaaggagttgaaggtgctggatttgacgatgaaaagtcactactaatgtcacaaataagcttagagaaacggtttggtaaatcagaagtatttgtggcttcgacgctcatggaaaatggcggtgttccacagtctgcaacccctgacaccctcctcaaagaagctattcatgttatcagctgtggttacgaggacaagaccgattggggagctgaaattggttggatttatggttcagttacagaagatattcttacgggatttaagatgcacgccagaggatggcgttcaatttattgcatgccaaagctcgcagcttttaagggttctgctcctatcaatctgtcagatcgtttgaatcaagtgcttcgatgggctttaggttctgttgaaattcttttcagtcgtcattgccccttatggtatgggtatggtggacggttgaaatggctggagagatttgcttatatcaacaccactatttaccctcttacatccattccacttcttgcatattgtaccttgccagctgtctgtctccttactggaaagtttattatccctcagattagtaacttggcaagtgtatggtttttatccctgtttctttccatctttgcaaccggtatcctggaaatgagatggagtggtgtgggaatagatgaatggtggaggaatgagcaattttgggtgattggagggatatcagcccatctgtttgcagtattccaaggtttgctaaaagttctagctggaattgataccaactttacagtgacttcaaaagcatcagatgaagatggtgatttcacggagctctacttgttcaaatggacaactctgcttatcccaccaaccactattctgatagtaaatcttgtagcagtggttgctggaatctcatatgcaataaacagcggttaccaatcatggggtccactttttggcaaactattctttgcattttgggtgattgtccatctttatcctttcctaaagggtctcatgggtcgccagaatcgaactcctactattgttgttgtttggtcgattctacttgcatccatcttctccttgttatgggttagagttgatccctttacaacccgggtgactggtccagacgttcatatatgtggaattaattgctagatggaggcgacaggtggaatggtggctggatcttacaaaaggaacgaacttgttagaattcgccatgatagtactgatagtgggtccaaatctttgaagaatttggatggacaaatctgtcagatctgtggggataccgtgggagttacttcgaatggtggtgtctttgtcgcttgcaatgaatgtgcttttccagtttgcaggccttgttatgagtatgagagaaaagatggaaaccagtgttgcccccaatgcaagacaagatacaagagacagaaagggagtcttcgagtgcagggcgatgatgaagaggaggatgttgatgatttggataatgaattcaattatgaaaggggaaccagcaaggctagacatcaatggcagggagaagatgtagatctctcgtcatcttctaggcatgaatctcagccaatccctctccttaccaatggtcaagtggtttctggggaaataccaagtgcgacaccagacaatcaatctgtgagaagcacatcaggtcctataggccctgagaagcgtggtaaccattcccttccttatgttgatccatgcctgccagttcctgtgagaattgtggacccttcaaaggacttgaattcttatgggattggtagtgtggactggaaggaaagagttgaaagttggaaactgaagcaggagaagaatatgactcatacgggaaatcgatattcagaaggaaaagggggcgatgttgaaggctctggttcaaatggtgaagaattccagttggctgatgatgtgcgtcaaccaatgagtcgtattgtccctatcccttcatcacatctcactccttaccgagctgtgatcattttccggcttatcattttagtcttcttcttgcaatttcgtattacccatcctgtggaggatgcatatccgctatggctcacatcagtcatctgtgagatctggtttgcaatgtcatggattttggatcagtttccaaaatggtatcctatcaaccgtgagacgtaccttgacaggctcgcattcaggcatgaccgcgaaggagagccgtcgcagttagctcctattgatgtgtttgtcagtacagtggatcctttgaaagagccgcctatcatcacagctaataccgtgttgtccattctagcagtggactacccagtcgacaaagtttcttgctatgtttcagatgatgggtctgcgatgctgacatttgaaggtctttctgaaactgctgagttcgccaggaaatgggtgccattctgtaagaagttcagtattgagccaagggcaccagaattctactttcaacagaagattgattacttaaaagacaagattcagccttcttttgtcaaggaacgcagagcaatgaagagggagtacgaagaatttaaggtccgaatcaatggccttgttgcaaaagcacagaaagagcctgaagaaggatggacaatgcaagatggaacagcttggcctggaaataaccccagggaccacccaggaatgatccaggtgtttttgggccacagtggaggtcttgacatggatggaaatgaattgccacgattagtttatgtttctcgtgaaaagagacctggcttccaacatcacaagaaagctggtgccatgaatgctttgatccgtgtttccgcagtactgacaaatggtgcatatatactgaatgtcgattgtgatcactacttcaacaacagcaaatgtcttaaagaagccatgtgtttcatgatggatcctgctcttggaaagaaagtatgctacgtgcagtttccccaaagattcgatggcattgatttacacgatcgatatgctaacagaaatattgtgttctttgatattaacatgaagggtcaggacggcatccaagggccagtttatgtgggaactggatgttgtttcaacagacaagcactttatgggtatgatccagtattgactgaagaagatttcgagccaaatttcatcatcaaaagttgttttggatcaaggaagaaggggaagagtggtaacaaaaagtacatggacaagaagaggggacctaaaagaagtgaatcttccataccaattttcaacatggaagacatcgaagagggtgttgaaggctatgaggatgagaagtctcatcttatgtctcagaagagactagagaagcgatttggtcaatcaccagtgttcattgcagctaccttcatggaaatggggggtattccacctacaactaatcctgccacactactgaaagaagccattcatgttattagctgtggatacgaggacaagtctgaatggggtaaagagattgggtggatatatggttcagtgacagaagatattttgactggatttaagatgcatgctcgtgggtggatgtcgatctactgcatgccaccaaggccagctttcaagggttcagctcccctcaatctttctgatcgtttgaaccaagttcttcgatgggctctaggttctattgagattatgcttagcaggcattgccccgtttggtatggctacaagggaaggttaaggtttttggagagattggcttacatcaacactgtcgtttatcccctaacgtctattccacttattgcctactgtattctcccagctatctgtctattgaccaataagttcatcattcccacgctaagcaactttgctagtatactgtttatcatgctcttcatgtccattgctgcaacgggtattcttgagctgagatggagtggagtatccatagaagactggtggagaaacgagcagttctgggttattggaggtacatcagcccatctgtttgccgtgttccaaggtttgctaaaggtgctagccggtatcgataccaacttcaccgtcacttccaaggcagccgacgaagacggtgattttgcagaactttacatatttaaatggacggcgcttcttatccctcccaccaccgtcctgatcgtgaacttggttggtgtagtggcgggtgtttcttacgccattaacagtggttaccagtcatggggcccactgtttgggaaactgtttttctcattctgggtgattgctcacttgtatcccttcctcaagggtctattgggtcgtcaaaacaggacaccaaccattgttattgtttggtccgttcttctcgcttccatcttctcgttgctctgggttcggattaatcctttcaccactgatgctgagaaggctgcagctggtaatcaatgtggtatcaattgctagatggaggcgactggtggaatggtggctggatcttacaaaaggaatgaacttgttaggattcgccatgatagtactgatagtgggtccaaatctttgaagaatttggatggacaaatctgtcagatctgtggggatacagtgggagttacttcgaatggcggtgtctttgtcgcttgcaatgaatgtgcttttccagtttgcaggccttgttatgagtatgagagaaaagatggaaaccagtgttgcccccagtgcaagacaagatacaagagacagaaagggagtcttcgcgtggagggagatgatgaagaggaagatgttgatgatttggataatgaattcaattatgaaaggggaaccagcaaggctagacatcaatggcagggtgaagatgtagatctctcgtcatcttctaggcatggatctcagccaattcctctccttaccaatggtcaagtggtttctggggaaataccaagtgcgacaccagacaatcaatctgtgcgaagcacatcaggtcctataggccctgagaagcgtggtaaccactcccttccttacattgatccatgcctgccagttcctgtgagaattgtggacccttcaaaggacttgaattcatatgggcttggtagtgtggactggaaggaaagagttgaaagttggaaactgaaacaggagaagaatatgactcatacgggaaatcgatattcagaagggaaagggggtgatgttgaaggctctggttcaaatggtgaagaactccagttggctgatgatgtgcgtcaaccaatgagtcgaattgtccctatcccttcatcgcatctcactccttatcgagctgtgatcattttccggcttatcattttagtcttcttcttgcaatttcgtattacccatcccgtggaggatgcatatccattatggctcacatcagtcatctgtgagatctggttcgcaatgtcatggattttggatcagtttccaaaatggtatcctatcaaccgtgagacgtaccttgacaggctcgcgttcaggcatgaccgtgaaggagagccgtcgcagttagctcctattgatgtgtttgtcagtacagtggatcctttgaaagagccgcctatcatcacagctaatactgtgttgtccattctagcagtggactacccagtcgacaaagtttcttgctatgtttcagatgatgggtctgcgatgctgacatttgaaggtctttctgaaactgctgagttcgccaggaaatgggtgccattttgtaagaagttcagtattgagccaagggcacccgaattctactttcaacagaagattgattacttaaaagacaagattcagccttcttttgtcaaggaacgcagagcaatgaagagggagtatgaagaatttaaggtccgaatcaatgcccttgttgcaaaagcacagaaggtgcctgaagaaggatggacaatgcaagatggaacagcctggcctggaaataaccccagggaccatccaggaatgattcaggtgtttttgggccacagtggaggccttgacatggatggtaatgaattgccgcgattagtttatgtttctcgtgagaagagacctggtttccaacatcacaaaaaagctggtgccatgaatgctttgatccgtgtttctgcagtactgacaaatggtgcatatatactgaatgtcgattgtgatcactacttcaacaacagcaaatgtcttaaagaagccatgtgtttcatgatggatcctgctcttgggaagaaagtatgctatgtgcagtttccccaaagattcgatggcattgatttacacgatcgatatgctaacagaaacattgtgttctttgatattaacatgaagggtcaggacggcatccaagggccagtttatgtgggaactggatgttgtttcaacagacaagcgctttatgggtatgatccagtattgactgaagaagattttgagccaaatttcatcatcaaaaattgttttggatcaaggaagaaggggaagagtggtaacaaaaagtacatggataagaagaggggacctaaaagaagtgaatcttccataccaattttcaacatggaagacatcgaagaaggtgttgaaggctatgaggatgagaagtcccttcttatgtctcagaagagactagagaagcgatttggtcagtcaccggtgttcattgcagctaccttcatggaaatggggggtattccacctacaactaatcctgcgacacttctgaaagaagccattcatgttattagctgtggatacgaggacaagtctgaatggggtaaagagattgggtggatatatggttcagtgacagaagatattttgactggatttaagatgcatgctcgtgggtggatgtcgatctactgcatgcctcccaggccagctttcaagggttcagctcccctcaatctttctgatcgtttgaaccaagtgcttcgatgggctttagggtctattgagattatgcttagcaggcattgccccatatggtatggctataagggaaggttaagattcttggagagattggcttacatcaacactgtggtttatcccctaacgtctattccacttattgcctactgtattctgccagctatctgtctattgaccaataaatttatcattcccacgctaagcaatttcgctagtatattgtttatcatgcttttcatgtccattgctgcaacgggtattcttgagctgagatggagtggagtatccatagaagactggtggagaaacgagcagttctgggttattggaggtacatcagcccatctgtttgccgtgttccaaggtttgctgaaggtgctggccggtatcgataccaactttacggttacttccaaggcagcggacgaagatggtgattttgcagaactttacatatttaaatggaccgcacttcttatccctcccacgacggtcctgatagtgaacttggttggtgtagtggcgggtgtttcttacgccattaacagtggttaccagtcatgggggccactgtttgggaaactgttcttttcattctgggtgattgctcacttgtatcccttcctcaagggtctattgggtcgtcaaaacaggacccctaccatcgtgattgtttggtccgttcttcttgcttccatcttctcgttgctttgggttcggattaatcctttcaccactgatgctgagaaggctgcagctggtaatcaatgtggtatcaattgctag

SEQ ID NO: 85 - DNA, 1737 bp, Spinacia oleracea; identical to SEQ ID NO: 62
  source 1..1737: mol_type = genomic DNA

SEQ ID NO: 86 - DNA, 1737 bp, Spinacia oleracea; identical to SEQ ID NO: 62
  source 1..1737: mol_type = genomic DNA

SEQ ID NO: 87 - RNA, 328 nt, Arabidopsis thaliana
  source 1..328: mol_type = genomic RNA
tggagaggaggtagctatcaagaagattggtaatgcttttgacaatattatcgatgctaaaaggactttgagagagattaagcttctcaagcatatggatcatgaaaatgtgattgctgtaaaggatataataaaaccaccgcagagagagaacttcaatgatgtttacattgtttatgagcttatggacactgatctccaccaaattattcgctctaaccaacccttaactgatgatcattgtcggttttttctgtatcagttgttgcgtgggctcaaatatgttcattcagctaatgtattgcatcgagatttgaaacctagcaat

SEQ ID NO: 88 - RNA, 322 nt, Arabidopsis thaliana
  source 1..322: mol_type = genomic RNA
ttgatgaatgagcttgcccttttcgaagagactctacaactctcttgtgtccctcctagaagttcagcaatggctttgaccacagacgaaaggtttttaaaagagcaaacacgagcaaacgacctaaagaccgtggagattggtcttcaaataagagagttaaggtgcaaagagacggcgctaggattaaaatttgaatcaaacaacctggggaaagcggcgctagagttggatgtttcgaaagctgcattcagagcggagaaattcaaaaccgaattagaagatacaagaaaagaagagatggtcacaagaatcatggatt

SEQ ID NO: 89 - RNA, 303 nt, Glycine max
  source 1..303: mol_type = genomic RNA
ctgtatcaattgttacgagggctcaaatatgtacattcagcaaatgttttgcaccgagatctaaagcctagtaattgctattgaatgccaattgtgaccttaagattgcggactttggtcttgctagaaccacatctgaaactgactttatgactgagtatgtggtcactagatggtaccgagctcccgaattgcttcttaattgttcagaatatacagcagccattgatatatggtctgttggttgcatacttggtgaaatcataacaagacaaccgctctttcctggcaaagattatgttc

SEQ ID NO: 90 - RNA, 258 nt, Glycine max
  source 1..258: mol_type = genomic RNA
attatctctctggagagtttcctgaggtgggttctgttgctgttaatcttggtcagattactctctctaacaaccagctttctggggttctacctccctccattggtaacttctccagcgtgcagaagctcattcttgatggcaacatgttcacgggtcggatacctccccagattgggaggttgcaacagctttctaagattgattttagtggcaacaagttctcgggtcctattgtgcctgagatcagtcagtgta

SEQ ID NO: 91 - RNA, 302 nt, Glycine max
  source 1..302: mol_type = genomic RNA
aggaatcttcctcctggtggactcccaagtcaatggtcttattcaattcaaagttgcatattttgtggtgtcaagttcaagttatgagccgaatggcatttggcatcttgatgatttttgctgttgcatatttgctcttgcagcggtcgaccacaacatcacagactatgccagtcactttcatccttttaatgttgggaattttttgtggctattgtggcaagctatgtgtagagacattgggtgggagtggtattgtgtggctcttatactgggagattatgtgcttgctgcatttctta

SEQ ID NO: 92 - RNA, 507 nt, Glycine max
  source 1..507: mol_type = genomic RNA
agtgttcatcgatgtattctggcctctaggagtaagtttttccatgaattattcaagagagagaagggttcatcagaaaaagaagggaaattgaagtataacatgagtgatttgttgccttatggcaaggttggatatgaagccttccccatattccttggctatgtatatactggtaaactcaagccctctccaatggaagtgtctacttgtgttgacagtgtgtgtgcccatgatgcttgtagacctgccattaactttgctgtggagttgatgtatgcctcttacatttttcaaataccagagtttgtatcacttttccagagacgtctacttaactttatagggaaggctcttgtggaagatgtcatcccaatcctcactgttgctttccattgtcaattgagtcaacttgtcaatcaatgcattgatagggtggccagatcagaccttgaccagatttcaattgaccaagagcttccaaatgaactctcacaaaaagttaaa

SEQ ID NO: 93 - RNA, 350 nt, Glycine max
  source 1..350: mol_type = genomic RNA
atggcgcagacgacccatcccatggacatcgacgaccacacttacaccgccagtcctgacaagggcaaaagtgtcgtcttcgccggaaaccctcccgccgccggcaaggccattccgtgggtcgaaaaataccgccctcagtccctcgacgacgtcgctgctcaccgtgacattgtcgatacaattgataggctaaccactgagaacagattgccccatcttctgttgtatggccctcctggcacgggcaaaacatcaacgattcttgctgtggcacgcaaactgtatggttcacagtaccaaaatatgattttggagctcaatgcatccgatgatagaggaattgatgt

SEQ ID NO: 94 - RNA, 366 nt, Glycine max
  source 1..366: mol_type = genomic RNA
cacttccctcgcgtgtatttatctggcacggatgattcccgatcatcatctaactctccatcaaaattggttgtggttaaagaggcatggtctcccattatcgattttgtggataaggccactgctgttagtgaagctggtgataaacagtctggtggatctcttgacccctctagctttcaagtcttgattgaagggctggctgaagttttagcagagaccaaatttgaagcagcaagcatggagcccttgaggaacatgttaatctttcagtaccttgtcgttgtttttgaggatgattttttacctcgcaatgcaaccttgaactggagcatgcagagggagtctttgctcagcctgctactg

SEQ ID NO: 95 - RNA, 328 nt, Glycine max
  source 1..328: mol_type = genomic RNA
cagtccttaccaatggacctttcttattgaatcttgattgtgatcactacataaacaacagcaaggccttgagggaagctatgtgctttatgatggatccaaaccttggtaaacatgtttgctatgtccagtttccacagaggtttgatggtattgataggaatgatcgatatgccaatcgtaatacagttttctttgatataaacttgagaggtttggatggcattcaaggtcctgtttatgtgggtactggatgtgtcttcaataggacagctttatatggttatgaacctcctcttaaacccaagcacaaaaagcctgggttgtt

SEQ ID NO: 96 - RNA, 400 nt, Glycine max
  source 1..400: mol_type = genomic RNA
catcccttacatcgacgaccccaaggaccgcgacgccgtctcacaggtctgccgacgctggtacgaactcgactccctcactcggaagcacgtcaccatcgcgctctgctacaccaccacgccggcgcgcctccgccgccgcttcccgcaccttgagtcgctcaagctcaagggcaagccccgagcagcaatgttcaacttgatacccgaggattggggaggccacgtcaccccatgggtcaaggagatttctcagtacttcgattgcctcaagagtctccacttccgccgtatgattgtcaaagattccgatcttcggaatctcgctcgtgaccgcggccacgtgcttcactctctcaagcttgacaagtgctccggtttcaccaccgatggtcttttc

SEQ ID NO: 97 - RNA, 365 nt, Glycine max
  source 1..365: mol_type = genomic RNA
ggcgagctgctttatctttctgaaatggatagaattgcagaggttgcactgaccaaggttggggaattcaactctcaaaacattgctaatattgcaggtgcctttgctgcaatgcagcattctgctcctgatcttttctcagagttttcaaaaagggcttcagacataattcacactttccaagaacaagaacttgctcagcttttgtgggcttttgcttctctatatgagcctgctgaccctatatttgattctttagacatagtcttcaaagatcatagccaattgagaggttgcataggtgaaaagacatcaaataatcatgaacaaataagtgttgatagaagtggtgcctcaaatggatc

SEQ ID NO: 98 - RNA, 279 nt, Glycine max
  source 1..279: mol_type = genomic RNA
gtgctgtccaacgtagcctttcaagggcatgccctcaaatatgtttcttcgtttggtttctgttgggagatcgaacagctccttcatggagccaaatacaccattttgtagctcaatagaagctttatcatacacagcaacaaagtaaccattctcctcaaatgcttggcgcacacttgtgcatgttgataaccaggaattggtccctggcttcaaatcttctatggtgaaatctagaacaggaagcataggttttccttcagaacccatcgtatgcct

SEQ ID NO: 99 - RNA, 201 nt, Glycine max
  source 1..201: mol_type = genomic RNA
tcacagactattgtatgcccggaatgaccggttatgaattactcaagagaatcaaggaatcatctactttcaaggaaacgcctgtggttattatgtcatctgaaaacgttttgccacgcatagacagatgtttggaggagggtgcagaggatttcatagtgaagccagtgaagttatctgatgtgaagcgtttgaaggatt

SEQ ID NO: 100 - RNA, 443 nt, Glycine max
  source 1..443: mol_type = genomic RNA
taagaaacatgaagaactgttgaaggacgcagagaaatgggcaaaggagacggcaacatccttcacaatagtggggactcttataataaccattgtgtttgcagcagccttcactcttcctggaggaaatgatcaaaatactggtattcctatgtttttgcacaaaagaatgttcacaacttatatggtggctgatgctatctcactcttttcttcctccactgctgtgatgacttttattggaattctcacatctcgttatgctgaaagagatttccttaaatccttgccactcaagttgatgtttggccttttcacactcatatgttctatcttggccatgatggttgcattttgctctgccttttctttgatgcttgaggattcgggtcattccaagatggtaaaatttgtcatttccattgcaagcttatctgtggtca

SEQ ID NO: 101 - RNA, 296 nt, Glycine max
  source 1..296: mol_type = genomic RNA
gggtattggaaggacacaggtgtcacggaacctatattgtcaacctttgataagaaagtgggaatgaagaagtacttggtgttccacataggtgaagctccacaaggtactgaaaccagttgggtaatgcaagaatattatatatgttcctctgggtttgacactgcctcatatcgaagtgctaggagaagaagaaaacatgatcaaagttggagcaagtgggttttgtgcaaagtctatgagaagatcaggtctcaacaaggtgtaaactgctatagcgatgatgatgacagtgg

SEQ ID NO: 102 - RNA, 360 nt, Glycine max
  source 1..360: mol_type = genomic RNA
gcttacttttgcggaccttcttgacgctaccaatggctttcacaatgatagtctcattggctctggtgggtttggggatgtttacaaggctcagttgaaggatggaagtgttgtggctatcaagaagctgattcatgtcagcggccaaggggacagggaattcactgctgaaatggagaccattgggaagatcaagcacaggaaccttgttcccctgttgggatactgcaaggtaggggaagagaggctcttggtttatgagtacatgaaatatggaagtttagaggatgttctacatgatcagaagaaagctgggatcaagctgaactgggccattaggcggaaaattgctattggagc

SEQ ID NO: 103 - RNA, 310 nt, Glycine max
  source 1..310: mol_type = genomic RNA
gcatagggtgggctattgtcaaagataaagaggtagcaaagaaaatgactaaattcatagagataagtacaatcggtgtctcgaaggattctcaactcagagctgccaaggttttaaaggcagtgtctgacagctgcgaacaagaaaactcccaacaagtggttccccatacgttttgtggatcaatttttgcttctaatgtcacatctcatgcttggagacacagctcaatttggacttcgtcgtccaaaacttggtcctctagagctcaagaacttgtacggaaagacaccagttttggatgttggga

SEQ ID NO: 104 - RNA, 399 nt, Glycine max
  source 1..399: mol_type = genomic RNA
actgctgtggtatcgtgtgcaactgccctcatgctggtacatattattcctgatttattaagtgttaaaactagggaattatttttgaagaacaaggctgcagagcttgatagagaaatgggcctgattcgcacacaagaagaaactggttgagggaagcttcttgcactgccaagtgtttgtgtatatataaaggctttggtcttgaagttgatgttgacaagtctttgcctgatctggtcattggtgatgaggcaaggactttccaagtaatagggtagtccagttgatgcaggggaacatatggttagtaccttgtaatcatggttttcctcaaagcatgactcttcttcttcggtttcaattacgaccgtccatttccatagccatctctgaccc

SEQ ID NO: 105 - RNA, 367 nt, Glycine max
  source 1..367: mol_type = genomic RNA
attatcatggtctgcccgagttcagaaatgctgtggctaaattcatgggtagaacaagaggaaacagagtcacgtttgatcctgaccgtattgtcatgagcggtggagcaactggagcacacgaagtcactaccttttgtttggcagaccctggtgacgcatttttggtgcccattccttattatccaggttttgaccgggatttgaggtggagaacaggaattaaacttgttccagttatgtgcgatagctcaaacaatttcaagttgacaaagcaagcattggaagatgcgtatgagaaggccaaagaggataatataagagtaaagggcttgctcatcaccaatccatcaaacccattaggcac

SEQ ID NO: 106 - RNA, 397 nt, Glycine max
  source 1..397: mol_type = genomic RNA
tgaaccatgggatgtgaatgatgcacatggccgaccattcacaacttttgctgctttttgggaaagatgtcttagcatgccttatgaccctgagtcacctcttctcccacctaaaaggataattccaggtgatgcatcgaggtgccctagtgatacattgctgtttgaagatgaattagagaaggcaagcaatgcacttgcacttctggggcacttaaagttttttccttggaatcctgatcctgatatctttaagacttggagacaaggtaggactggctttcctttggttgatgcaggaatgagagaactttgggcaacaggatggatacacaacagaataagagtaatagtttccagttttgctgtgaaaatgttgcttctaccctggaaatgg

SEQ ID NO: 107 - RNA, 376 nt, Glycine max
  source 1..376: mol_type = genomic RNA
aatggtgaccagcaaaggtcttgagtcagttcagtcagaaatcaatgacttggactgggaaagcacctttttcctgcgccatcttccactctccaacgtttcagacaacgcagatcttgatcaagattacaggaagacaatgaagaaatttgctctggaactggagaaacttgcagagcagcatggagcaaaggttccaagaaatggtagcaagcaaaggcttggagtctgctcaatctgaaatcaatgatttggactgggaaagcactttctttttgcgccatcttcctgtctctaacatctcagagatccctgatcttgatgaagattacaggaaggtaatgaaggattttgccgtggaactggagaaactggc

SEQ ID NO: 108 - RNA, 390 nt, Glycine max
  source 1..390: mol_type = genomic RNA
aacgtttacggcatccaaacattgttttgtttatgggggcagtcactcagcctcctaacttatcaattgtcacggaatatttatctaggggtagcttatataggcttttgcatagatctggcgccaaagaagtattggatgagaggcgtaggcttggtatggcttatgacgtggcaaaggggatgaattactgaagctctgggtgcaaattatgcgggtgggaacagccatctgattgctttgattcctggtgcagaggaatatgagtctttcaatgaagcaaaccagtcaataatggattacccaagtcatgaagttgatcttgacaaggaggatctggatattccatggagtgaacttattttgaaggagaacattgggacagggtca

SEQ ID NO: 109 - RNA, 421 nt, Glycine max
  source 1..421: mol_type = genomic RNA
ccagagcttctgttgaactcttctgattacactgcagcaattgatgtatggtctgttggttgtattttcatggaactgatggatcgcaagcctttgtttcctggcagagatcatgtgcatcagttgcgtctacttatggagctgattggcaccccatcagaggctgatttggggttcctgaatgaaaatgctaagagatacattaggcaactacccctttaccgccgtcaatctttccaggaaaagtttccacatgtccatcctgaagctatagatcttgttgaaaaaatgttaacttttgatcctagaaaaaggattactgttgaagatgcactggcgcacccgtacttgacatctctgcatgacatcagtgatgaaccagtgtgcatgactcccttcaactttgactttgaacaacatg

SEQ ID NO: 110 - RNA, 243 nt, Glycine max
  source 1..243: mol_type = genomic RNA
ctacctcctaccagacctagaactaattcagaggatcaatccggaaaaggatcactgcatttggaagatgatcccaagaattttatcgaatctccagctcgtcatgacaatgtaagcagtaggagtggcagtagaagccatggagtaggttctgctgataaccgtagaagacattcaacgcaaagtactgggtctgagtacagcattgaacgttcaccgcttcatcgccaggctagagcccca

SEQ ID NO: 111 - RNA, 159 nt, Glycine max
  source 1..159: mol_type = genomic RNA
ggtgactgggatgtgaataacccatcatcagcagatggctttactcacattttcaacaaagtgagggaggagagacagggtgtcccaggacaagtgccgggaacgcctaatgagagaccacaagctattaggggccaatctaacgatgacaaagtccag

SEQ ID NO: 112 - RNA, 299 nt, Glycine max
  source 1..299: mol_type = genomic RNA
aaggttccttgatatggggccatgggctgtagatctggcattatctgttggaatattcccatatgttctaaagctgttacaaacaacaacaccagaactacgacagatccttgtattcatatggacaaagattcttgcacttgataagtcatgtcaggttgatctagtgaaggatggtggtcatatctattttattaagtttcttgacagcatggaagcatatccagagcaacgcgcaatggctgcttttgttttggctgtgattgtggatggtcatagacgaggccaagaagcttgta

SEQ ID NO: 113 - RNA, 297 nt, Glycine max
  source 1..297: mol_type = genomic RNA
gacccaggcttgggatatttattatcatgttttcagaaagatagataagcaacttcaaagtcttacaactttagacttggagtctgtttctccagaattgctagaatgccgcaatttggagcttgctgttcccgggtcataccgtgctgatgcaccagtggttacaattgcatcatttgcacgccaacttgttgtcataacatccaagcaacgacccaggaagttaaccattcatgggagtgatggtgatgattatgccttcttattgaagggacatgaggatttacgccaggatga

SEQ ID NO: 114 - RNA, 360 nt, Glycine max
  source 1..360: mol_type = genomic RNA
aagggggagaagcattgaaggactcaaattccctaaaagtcttgtttgtgtggctggtttggatcttctgcaggattggcaattggcatatgcaaaaggtcttgaggattgtggacaacaggtcaagcttctttttctcaaggaagccactattggcttctacttcttgccaaacaatgaccaggagaagaacgttcggtgaacattcttgaccttgagaagcctgtgagctctgaggttgttcctgtcatcatatttttccatggtggaagttttgcgcattcttcagccaatagtgctatatatgacaccctttgtcgtcgcctagtgggtatctgtaaggctgttgtggtgtctgtg

SEQ ID NO: 115 - RNA, 350 nt, Glycine max
  source 1..350: mol_type = genomic RNA
gcatcgcatggttgatgtagttgttacaactacaggtggcattgaagaagatcttataaagtgcctggcaccaacatataaaggagatttctctttgcctggagcatatctgcgctcaaaagggttgaatcggattggtaatttattggtcccgaatgacaactattgcaaatttgaggactggattattcctatctttgatcagatgttgggggaacaaaataatgagaatgtgttgtggacaccatctaagttaatagctcgattgggaaaagaaataaaaaatgaaagctcttacctttactgggcatacaagaacaacattccagttttctgtccgggcttaactg

SEQ ID NO: 116 - RNA, 339 nt, Glycine max
  source 1..339: mol_type = genomic RNA
tgtggtctgagggattcacaattgttggatcccctcttgagcattttcgtcaactctggccccaagattaccacaaatactgtgatatcgtcaagcgatatgatgaagccatgaaaaagctagtgggaaagctgatgtggctgatgttggattctctgggtattacaaaggaagacctgaaatgggccgggtccaaaggccaattcaaaaagacatgcgcagccttgcaattgaactcttacccgacttgtccggatccggatcgggccatgggtctggccgcccacaccgactccacccttctcacaatcctttaccaaaacaacataagcgggttgc

SEQ ID NO: 117 - RNA, 300 nt, Glycine max
  source 1..300: mol_type = genomic RNA
ttcacgcatgctgcttttacttttggttatgaagctggtattaacaaatgctccattgatggaaatttggtgccgtcaggtgctcttgttacatttattcagaagggtctacagtactttgagatggaagccaacttgagtaattgtgatgcagacctggaagaagatttttcattcttacaacctttggatcttatcacgaaagatgtgcatgaacttaggcaaatgataagtgaaagaagaaaaaaactacaaaaggaaagaaagaaggaatttgaaaaggagcatgaaggtgaacgt

SEQ ID NO: 118 - RNA, 362 nt, Glycine max
  source 1..362: mol_type = genomic RNA
ggagcctccaagacctatcctcaacaagccggaaccatccgcaaaaacggttacatcgtcatcaagggtcgtccctgcaaggttgttgaagtttctacttccaagactggtaagcacggtcatgccaagtgtcactttgttgggattgatattttcactgccaagaagcttgaagatattgtgccctcttctcacaactgtgatgttccccatgtgaatcgtactgattaccagttgattgacattgctgaggatggatttcttagcttgcttactgaaaatgggaacaccaaggatgacctgaagcttcctactgatgagagtttgcttactcagataaaggatgggtttgctgagggtaa

SEQ ID NO: 119 - RNA, 243 nt, Glycine max
  source 1..243: mol_type = genomic RNA
atggcgcagagagctcaaccagcgttggccaccacggatgtttttcagaagggagatattcattggaagcaattcaatgttgaaacatccactgcttcttcctcacctccaaaaccattgcttatttttacaccaaccgtgcctggcttataccctgtaatattgttttgccatggcttttgtattcgcactagctactactctaagctcctagcccacatagtttcacatggattcatactt

SEQ ID NO: 120 - RNA, 287 nt, Glycine max
  source 1..287: mol_type = genomic RNA
gctcatgtggtggttgagattccaagcaatacgacggagagcaatacaaacgagaagcaaaatgagttatccactacctttgagattggaaaggctgaagtaatacgtggccaagggcactgtaagctttgtgacaagagaatttcttgcagaactgcagtaggcaggtctttggtatacagacctgcaatgctctcaatggtggcaatagccgctgtctgtgtctgtgtggcccttctgtttaaaagctcccccgaagttatatgtatgttccgacccttcaggtg

SEQ ID NO: 121 - RNA, 307 nt, Lactuca sativa
  source 1..307: mol_type = genomic RNA
tttgttgctcaggtgttggaaaagaaagaagcaactggagaaaagatgtctatagatcatctctcccagatatgcactttggcagttaaagaatcattgtctcatgtttttggcaacaaatttgattactttgtaagcaacttcgaaagatcatttcagagcaccttgatgacactcagagtaatcaatgaaacatcagaaaacagtaaaagatcatatcaacacgtggaagggagttcaacctatgattttcattttgatatgaaagaaaacacagccacaagccaagaacaagccaccattgttt

SEQ ID NO: 122 - RNA, 344 nt, Lactuca sativa
  source 1..344: mol_type = genomic RNA
ccgtcctcgatgttgtggctttgttgctcaggtgttggaaaagaaagaagcaactggagaaaagatgtctatagatcatctctcccagatatgcactttggcagttaaagaatcattgtctcatgtttttggcaacaaatttgattactttgtaagcaacttcgaaagatcatttcagagcaccttgatgacactcagagtaatcaatgaaacatcagaaaacagtaaaagatcatatcaacacgtggaagggagttcaacctatgattttcattttgatatgaaagaaaacacagccacaagccaagaacaagccaccattgtttgaccaggatgggcaccac

SEQ ID NO: 123 - RNA, 348 nt, Nicotiana benthamiana
  source 1..348: mol_type = genomic RNA
caagtgcaaatgctcttcctgaaaagtacaggatgctaatgatggattcatcatcgccaatttctgatttttacccaacagattttgaacttgacatgaatggaaagcgttttgcatggcaggctgttgtaaaattgccgttcatcgacgagaagaagttacttgctgaaacgaaaaagcttgaagacacattatcggaagatgagcaactcaggaacagtgtactgttcgatttgctctatgttcatcatcaccatactttggctccatatatcatttcgtactatcatcacaataataaggtgcctccaggggaacaaaggccatggctaattgatgcaaatctga

SEQ ID NO: 124 - RNA, 348 nt, Nicotiana benthamiana
  source 1..348: mol_type = genomic RNA
aatctgctgatgtgcatctatagaagagagtttacaacaatgggtggttatcttacagatgcgggtgaggtttttttggatagagtagaacattttattcaggcggcagcagtttatgaagaccaaatctttcaaaagagagctaggataaagcagtcaatagaaaataatgaaagagcaagaaatgaggttaatgctcagccacagcctccggcagaggacaaggtgaagttaggggagcctggatacaaggaaaggtactattccgagaaatttggtgtatcaactccagagggtattgatgaagtcagacaagatgtagtccagaagtatgtggaaggattatgt

SEQ ID NO: 125 - RNA, 300 nt, Nicotiana benthamiana
  source 1..300: mol_type = genomic RNA
ctcattagtccgtccaagaaaactcctctatatggctattgatggagtggctcctagagctaagatgaaccagcagcgaaccaggcgttttagagccgctaaagatgccgctgaagctgaagctgaagaaaaaaggttgagggaagagttgtaaccttcctggcttcgacccaaatacccgtcattgtctttatggcctagatgcagatctgattatgctgtctcttgctactcatgaagtccatttttcaatattgagggaggtgattacatcaccaggtcagcaggagaagtgttttg

SEQ ID NO: 126 - RNA, 347 nt, Solanum lycopersicum
  source 1..347: mol_type = genomic RNA
ctccatttgcttctgatctcaaaggtctagcagatttggaaattacttttttcccaggggaaccatttaagcccttcgatcagctaatgggtgttctaccagctgcaagtgcaaaagctcttcctgaaaaatacaggatgctgatgatggatccttcatcaccaatttctgatttttacccaacagatttcgaacttgacatgaatgggaagcgttttgcatggcaggctgttgtaaaattgccgttcatcgatgagaaaaagttacttgctgaaacaaagaagcttgaagacacgttaacggacacggagcaactaaggaacagtgtaatgttcgatttgctctat

SEQ ID NO: 127 - RNA, 308 nt, Solanum lycopersicum
  source 1..308: mol_type = genomic RNA
caacagttggcttctgcttcttcaagcatgattttatccaatacagggattagccaatctgtgcataggactttggaaagatctctcacagaacaagctaggtcgaatgaccttaagacatttgagattggtcttacaatgaggaagttgcaacttaaagaaaggcagctggctctcagttctgatgctaaccttttggagagggtcaaactatcatttggtttctctaaaacgtccttcaaaactgaaaagttcaaaaatcaagtagaagactcaaaacatgctgagctacttaaaacctgtataga

SEQ ID NO: 128 - RNA, 345 nt, Solanum lycopersicum
  source 1..345: mol_type = genomic RNA
ccgtcctcgatgttgtggccaacagttggcttctgcttcttcaagcatgattttatccaatacagggattagccaatctgtgcataggactttggaaagatctctcacagaacaagctaggtcgaatgaccttaagacatttgagattggtcttacaatgaggaagttgcaacttaaagaaaggcagctggctctcagttctgatgctaaccttttggagagggtcaaactatcatttggtttctctaaaacgtccttcaaaactgaaaagttcaaaaatcaagtagaagactcaaaacatgctgagctacttaaaacctgtatagagaccaggatgggcaccac

SEQ ID NO: 129 - RNA, 328 nt, Spinacia oleracea
  source 1..328: mol_type = genomic RNA
caaccagcacaaccccaacaacaatcatggaatttcgatcaaaagaatgatggatcttcgaactcaattacagctatggtgaaatcagagtttcctggtgagaattacaaccaaggaagcgttgtaaagtatgagacagaccaaaaccctccctaccaaccagctcaatacataagagaacagagaagatcagaagatgggtataattggagaaaatatggacaaaaacaggtaaaagggagtgaaaatccaagaagttattacaaatgcacgtttcccaattgtccaatgaaaaagaagattgagacagcaatagatggtcagatta

SEQ ID NO: 130 - RNA, 328 nt, Spinacia oleracea
  source 1..328: mol_type = genomic RNA
ctttggtggtgagggattcaattggactcaactaggtgctaataaggatgaaggaagaaacaacaattttactttccatgcttcttcaaacaactcaattgaagggtggaatttggatgaaccgaagaagcaaaatgagatgattaacaacaaagatcaaatcaattcaaaatccgagtttactactgttaatcaaaccatgccatatgaaatgaacacaacaaaaacagatgttggtactaatatgcaaagagggtacaatgctcaaccagttcagatgaatcgagcacagaaaagagctgaagatgggtattgttggaggaaatat

SEQ ID NO: 131 - RNA, 324 nt, Spinacia oleracea
  source 1..324: mol_type = genomic RNA
gacagtcgcacaaccacccgatgactttggatttcacaaaaccgaacccgaatacaaaaccatgtgcgtcgatttcgaattcggcgtcctttagtatatcttcatcggcgaactcatcgtcgtttttgtctactattaccggagaaggaagcgtgtctaacagaaagctattcggtggatctgtttttgttccacctcctacatcgtcgtctgggtttgctgcttctgggaaacctcccctttcaacgtcttcgtcttctcatcataataagagttgtcaccaccaccaccaagagttttccgaagacttttccggtaaaatct

SEQ ID NO: 132 - RNA, 313 nt, Spinacia oleracea
  source 1..313: mol_type = genomic RNA
aaagagaaagaggaaaaagctggaaaagtaaaggatgaaaaatctgggaaagagagggaagaaaaagcagtgaaagagaaggaagaaaatgttgggaaagatagggaagagaaatctatcaaagaggaggaggaaaaacccaagttacaagatgtgccttctgatatggaaacagttgttgagcgagaggagaagcctgccatcaaatatgaagagaatagagtttgtcctgatcctgagcccatggatatctctccatcatcaccttccatggagtgtgaaatttctacttcagatgtgacaatattagagg

SEQ ID NO: 133 - RNA, 276 nt, Spinacia oleracea
  source 1..276: mol_type = genomic RNA
ggaggaactttaccccgcctaccatacctgaatgtgatgaatctgactgctctactattgattccggacagtggattagcttccaaaggttttccagacctccaattccaaattcccccacatttaacttggttaggccaatggttcagaaaatgtccttgaatgatgcaattcaggatagagcaagagttgctgaagctgagagtatgcaggttaccccttgggagggggaaaggattcatgatgtaggcatggatgatcttgaactcactcttg

SEQ ID NO: 134 - RNA, 328 nt, Spinacia oleracea
  source 1..328: mol_type = genomic RNA
ctataatcaaggtaataatgatgaatcaaagaatctttttgcggaacttaatccattccatttacgagtttctggccaagcttcacagcaaaataaacccaaatacgagattgatgagtctcagagatttaagaatgatgctgtctccagaaaaccgccttctcccatgatgtggaaaaaccaccaggctatcaattatgtccccaggaataaagattatgatattgttgaaagtcaactcccgagaaataacaatggaaacaaagattccagagcggcaccagtaacttcatctagaacatcagcttcccaaaatttacgtcaaaat

SEQ ID NO: 135 - RNA, 246 nt, Spinacia oleracea
  source 1..246: mol_type = genomic RNA
gcagctcaggaagcaatgaacaataccgtacgcagagcaagtaaagccatcacagaacaagaagcaaggcagattcttggcgttcctgagaatgcggcctgggaagatattttgaaaaaatatgatgttttgtttgagaggaatgccaagcaagggagcttctacatccaatcgaaggtccatagagcaaaggagtgtctagaagcgatacatcaaagcaaaggtcaaggttccgccaatgggtaa

SEQ ID NO: 136 - RNA, 288 nt, Spinacia oleracea
  source 1..288: mol_type = genomic RNA
gttaattcgggcggttggtcaagcttatcgtcaagctattgtcaatgcctcaaaaactggtgctgcgcaagaagcaatgcaaaacaccattcgtagagcaagtcaggctcttacagaacaagaagcaagacagatacttggagtccccgagaaagccgcttgggaagaaattttaagaaaatatgatgttttatttgagaggaatgctaaacaagggagcttttacctccaatcgaaggtacatagagcaaaggaacatctggaagaagtgtatcgaaataaaggtta

SEQ ID NO: 137 - RNA, 323 nt, Spinacia oleracea
  source 1..323: mol_type = genomic RNA
tatcaggccaggtggtagagagaaactcaaaaacagcacaacctactttgattgtggatttggggatgagtgtgctaagtgctgactctgtaccacccaagttttctgaatcatctgtgagccctagggaaaatcagattttgatgtttgagagaacaaatgataataagcttacaccaactggtactctagaggaaaactctgaaaaggagtgtcatgaagatgtatctgcagcaaaagacgcaaaaaatgttgaatcacgtgaggttaagaaaacttccgacaagaaatcaagaaagctaaagtcttccaaggcgcaacca

SEQ ID NO: 138 - RNA, 327 nt, Spinacia oleracea
  source 1..327: mol_type = genomic RNA
ataatggtgagagtagttccagtgatgaagaggattcctatggaaaacctaaagaagagactattaaagctaagatcacaagggttgctgttcgtactcaagcttctgattccacccttattcttaaggatggataccaatggagaaaatatggccaaaaagttacaagagataacccttgccctagagcttacttcaaatgctcctttgctcctggttgccccgttaagaaaaaggtacaaagaagtctagaagaccagtcaatgttagtggcaacgtatgaaggagagcacaaccaccaaccaccagcacaacaagagggtcagg

SEQ ID NO: 139 - RNA, 326 nt, Spinacia oleracea
  source 1..326: mol_type = genomic RNA
atgacaaaacacaggtggacgagagaaaaagtgggaatttagaagaggagttgaacaggatgaacatagagaataagaagctgacaaaaatgttggcatccatgtgtaagaattacagtatgctgcaaaatcagttgattggtttgattagtccgacgtcgaaaggcgaaaatgaagatcagaatccaaggaagaggaagtttattgaagctgtggagtgtggaatacaggggaacacagaacagagttgtattagctacgatgattcattcaaaagagtaactcttcttagcagcaagcctaagatcactaataaaatgttgatt

SEQ ID NO: 140 - RNA, 326 nt, Spinacia oleracea
  source 1..326: mol_type = genomic RNA
gacttgtaaccttctcagccagtatgttaaggagaaacgcgctcaaggtgttactggaaacatcgagtcccgaggaccaacttcaacgatgaatttgttccctgtgagtgggaaacatgcagctgaagattcaaatgtttgtgctgggaaaacaatcaatctctttcctcagctttctgggtttaattcaagcgtgaacaagcgtgctaaccccgagcctccgactccccaaatgaccatcttctatggagggcaagtcttggtctttaatgacttgccgactgacaaggcaaaagaagtcatggacctagcttgtagctttgaag

SEQ ID NO: 141 - RNA, 327 nt, Spinacia oleracea
  source 1..327: mol_type = genomic RNA
tatcaccatcttccacgtcgatctcttcaacagcttctacgatctccatgggatctgttaatggagataatccaaaaaagcaagaacaacaaaggacgatattttataatggaactatctgcgtttgcgatgttaccgatgttcaggcaaaaagcataatacagttagcaagtagaaaaacgatacaagaaatatgtacatctacaacaataacaccaacgttacaatctcaacaattatgtactcaaacgcaaaatggtttatcggccatgaagaaatcgttacgccaatttctcgaaaagcgaaagcatcgaatccaagctacct

SEQ ID NO: 142 - RNA, 328 nt, Spinacia oleracea
  source 1..328: mol_type = genomic RNA
ctggggaagatagcatggagaaagcagaagaaactgccccaatgaccatattctacaatgggtcagtttctgtttatgatctacccaacgacaaggccgaaagcataatgaagcttgcaatggaaaagaatgaatcaaaaaacgaagggaaggtggtggattattcatgttttaagtcatcccaagcaactactaagaatatcctcggacgacttaatgatgatgatggagatgattctgatatgcctattaaacgtaagaagtcattgcagaggttctttgagaagcgcaaagagaggttggtgtcgccatatacaaacatagaaca

SEQ ID NO: 143 - RNA, 326 nt, Spinacia oleracea
  source 1..326: mol_type = genomic RNA
ctcctcgaagggaggcatttaatgatgtatatattgcatatgagctgatggacactgatcttcatcaaattatccgttcaaaccaaggattatcagaagagcactgtcagtattttctctaccagatactacgtggattgaagtacattcactctgcaaatgttctacacagggatctaaagcctagcaatctcttgcttaatgccaactgtgatttaaaaatctgtgattttggactagctcgagtaacatcggagactgattttatgactgaatatgttgtgactagatggtatcgggcaccagagttattgttgaattcttca

SEQ ID NO: 144 - RNA, 328 nt, Spinacia oleracea
  source 1..328: mol_type = genomic RNA
aagacggtaggaaaaaggactttagaatgggatttgaatgattggaaatgggatggtgatctttttactgccagtccattgaattctacacctttagattgtagaagtaagcagctttttcctcttggaccagaacttgtggctaacaccaatgtctgttctaaagaaattaataaaactaatgagaaagggaaaagggaaatggagaagagaaggaggggtgtggttgttcccgaggaggagttgaatgatgaagctagaccactcaatttgaagttgggtgaacaggtttatcctatagcggaaggtgaagctgataaattggaag

SEQ ID NO: 145 - RNA, 328 nt, Spinacia oleracea
  source 1..328: mol_type = genomic RNA
gaatggtggctggatcttacaaaaggaacgaacttgttagaattcgccatgatagtactgatagtgggtccaaatctttgaagaatttggatggacaaatctgtcagatctgtggggataccgtgggagttacttcgaatggtggtgtctttgtcgcttgcaatgaatgtgcttttccagtttgcaggccttgttatgagtatgagagaaaagatggaaaccagtgttgcccccaatgcaagacaagatacaagagacagaaagggagtcttcgagtgcagggcgatgatgaagaggaggatgttgatgatttggataatgaattcaa

SEQ ID NO: 146 - RNA, 328 nt, Spinacia oleracea
  source 1..328: mol_type = genomic RNA
atttgagaggtttagatggtatccaaggacctgtctatgtgggtacgggatgtgtcttcaacagaactgctttgtatggttatgagcctcctatcaaaccaaagcccaagaagaaaggtatcctatcatcatgctttggtggatctcggaagaaatcaagcaagaaagactccaagaagaaatcaaaacatgctgaccctactgtgccaatatttaatctggaagatatcgaggaaggagttgaaggtgctggatttgacgatgaaaagtcactactaatgtcacaaataagcttagagaaacggtttggtaaatcagaagtatttgt

SEQ ID NO: 147 - RNA, 328 nt, Spinacia oleracea
  source 1..328: mol_type = genomic RNA
gaatggtggctggatcttacaaaaggaatgaacttgttaggattcgccatgatagtactgatagtgggtccaaatctttgaagaatttggatggacaaatctgtcagatctgtggggatacagtgggagttacttcgaatggcggtgtctttgtcgcttgcaatgaatgtgcttttccagtttgcaggccttgttatgagtatgagagaaaagatggaaaccagtgttgcccccagtgcaagacaagatacaagagacagaaagggagtcttcgcgtggagggagatgatgaagaggaagatgttgatgatttggataatgaattcaa

SEQ ID NO: 148 - RNA, 328 nt, Spinacia oleracea
  source 1..328: mol_type = genomic RNA
taagctcacaaattgttgtttcaaatatgggaaatcacttgagcaactattcagtgctcagtaccattgagaaatctgttgttgagcagacgcgtgcaaatgacctaaagagcgttgaaataggtctaattatgaaaaagttgcggctgaaggaaactcaactggctcttaattctgatttgaatttgttggagagatgtaaattgtctctaggaatttcaagaacatccttcaaagtcgagaagtttaagaatcaattggaagagacaagacatagtgagctcctcagaacatgtgcagactttcttgttgctgatttgcttataat

SEQ ID NO: 149 - RNA, 326 nt, Spinacia oleracea
  source 1..326: mol_type = genomic RNA
ctcttgcgtatcactagctcttgcagaaacctgagaacactgtttctggaagacagtggtgtcgaggagactaatggtgaatggtttcaccagctggcactacacaatatagttcttgaaactctgaacttctatatgacttttatggagaaaatcacagttgaagatcttgaacttcttgccagaaattgtcctttagtctctgtgaaaattggagatactgatcttgttcatcttggaaaatttcttcgtacagcgacgagattgcaagagttctccggtggttcttataacgaagatttagaccaatatggtaatattgtatt

SEQ ID NO: 150 - RNA, 328 nt, Spinacia oleracea
  source 1..328: mol_type = genomic RNA
aacaacgcttcataacatccaatctttcactaatctcttgtggcctactaggaacgatcatttttgtgaaaccatccatgaatatgcaaagacagtatcggagctagaacaattggtaatgaggatggtatttgagacttacggggtggagaaataccttaactctcatctcaactcgaccacttaccttcttcgtatagcaaagtacagggtaccactcaaggatgaacttaatcttggtgcagttggtcacactgacaaaaacttcataacaatacttcatcagaatcaagttaatggtttggaagttcaaactaaagatggtgaa

SEQ ID NO: 151 - RNA, 327 nt, Spinacia oleracea
  source 1..327: mol_type = genomic RNA
gctcgaaaggattgaatcgcattggtaacttattggtcccaaatgacaactactgcaaatttgaggattggattgttccaatttttgataaaatgctggaagagcaaaatacagagaagataacatggacaccctcaaagttgatcgctcgtttgggaaaagaaataaatgacaagagttcataccttttttgggcctacaagaataatattccagtattctgcccaggtttaacagatggatcacttggagacatgttgtattttcattcaatacggaatcccggtttaattgttgacgtagttcaagatataagagccatgaatg

SEQ ID NO: 152 - RNA, 281 nt, Spinacia oleracea
  source 1..281: mol_type = genomic RNA
cagatgaggttacaattccgatcatatgatcctatccaacctatctcattcgttctactacaaaggaatttcaggtgtagaaatatgggtgatagtgatattttggcgtcggttcgctctgtggtgttcaaggagtcagaaaccctagaagggaaatgcattaagactgagagttatacccagctttttaaatcgtttgtctccaccggtttccaagcatcgaatctcggagaagctattgatgttgttaatgaaatgattgacgactggaaaatgagata

SEQ ID NO: 153 - RNA, 326 nt, Spinacia oleracea
  source 1..326: mol_type = genomic RNA
tctaagactggcaagcacggacatgctaaatgtaactttgtcggtatcgacatcttcactgggaagaagcttgaggatattgttccatcttctcacaattgtgatgttccccatgtcacccgtactgattaccagctccttgacattactgaggatggttttgttagcctgctgactgataatggagatactaaggatgacttgaagctgccaactgatgaagttctgaataaacagatcaaggatggatttgctgaggggaaagacctgattgtgagcgtgatgagtgccatgggagaagaacaaattaatgccctcaaggacat

SEQ ID NO: 154 - RNA, 328 nt, Spinacia oleracea
  source 1..328: mol_type = genomic RNA
agcacggtcatgctaagtgtaactttgttggtattgacatcttcaatgggaagaagcttgaagatattgttccatcttctcacaattgtgatgttccccacgtcaatcgtaccgattatcagctcattgacatagctgaggatggttttgttagcctgctgactgagagtggaggtacaaaggatgacttgaagctgccaactgatgaaactcttcttaagcagctcaaggaaggatttgcggatggaaaagacctgattgtgagtgttcagaatgccatgggagaagaacaaatttgtggcgtcaaggatattggtggtggatctaa

SEQ ID NO: 155 - RNA, 328 nt, Spinacia oleracea
  source 1..328: mol_type = genomic RNA
ctttccctcagcaagctggtactatccgtaagaacggttacatcgtcatcaagaatcgcccctgcaaggttgttgaagtttccacttctaagaccggcaagcacggccatgccaaatgtaactttgttggtattgatatcttcaatgggaagaagctcgaggatattgttccatcttctcacaattctgatgttccccatgtcactcgtactgattaccagctccttgacataactgaggatggctttgttagcctgctgactgacaatggagacactaaggatgacttgaggctgccaactgatgatgctctgctgaaacagcttaa

SEQ ID NO: 156 - RNA, 327 nt, Spinacia oleracea
  source 1..327: mol_type = genomic RNA
aaagcagtaatcttcttgcgactctgataaatgatgttttggacctttcacggcttgaagatggaagtttgaagcttgataatggagtatttaatctccacacgatgtttagagaggtccttaatttgatcaagcccatagcatctatcaaaaagttgttgataacgacgaatttggcaacggatgtgccagagtttgctgttggtgatgaaaaacgtctaatgcaggttctattgaatgttattggtaatgctgtgaagtttacgaaagaaggaagtgtctcagttactgctttagttgcaaaagcagaatctttaagagatgctc

SEQ ID NO: 157 - RNA, 328 nt, Spinacia oleracea
  source 1..328: mol_type = genomic RNA
cggctactgtcgaggagtacattaaagtaaagtttgagtttgacataagtggaggttatctgacagacactattaggagaacccagaatgattcaaacaaggaagtattgagcttcagcatgtgcaaaaggcttgctcagatgatgcagggtgatatatggatatctctaaactcaaatgggataccacagaacatgactcttgttctacgatttcaacaacaaagatatattcctgaaggaaacttatatatgacagagcacacagtttcttcaaactctcggttcagagggttgaaagtgttgattgctgatgatgataatgtaaa

SEQ ID NO: 158 - RNA, 328 nt, Spinacia oleracea
  source 1..328: mol_type = genomic RNA
ttgtgctactgccattacccttatcaccctcatccctttgcttctcaaagtcaaggtcagagaattcatgttaaagaagaagacttgggatttgggtagggaggttgggatgattaagaaacaaagtgaagctggatggcatgttaggatgcttactcatgagattcgcaagtccttagataggcatacaatcttgcacacaactatggttgagctctctaaaaccttggatttgcttacttgtgctgtttggatgcccaatgagaataggactgagatcattttgactcatggtttaaaaggaaggaattttcagagtttcagcaac

SEQ ID NO: 159 - RNA, 328 nt, Spinacia oleracea
  source 1..328: mol_type = genomic RNA
ttctacaggtgcagttgtgaatgagcaagatgcaggagatcctatgtatcttaagcaacttggtagaaagctgcccgacgtaaacaatcctataccaaatgaccaagtacattctcagtttgttggtcatgatagagaaccccagtgttctgaatctagtaatcctccacaaaatattggaagagcatctaatgccatcagagagtttcttccatcaagatttgttcctcctagcagacgagggacccaggcattaactgattcagcgagaaatgcatcaaaagattttcggtttgaggctagtcaactagtttcaagtagaagtgaa

SEQ ID NO: 160 - RNA, 326 nt, Spinacia oleracea
  source 1..326: mol_type = genomic RNA
ctggccttgtttgtgctgctgttaattcagaaacacaccatgaaattgctatcaagaagattggtaatgcattcgacaatatagtagatgcaaaaaggacactgagagagatcaaactacttcgccatatggatcatgaaaatattattgcgattaaggatatcattcgaccaccaaagagagaagcattcaatgatgtttacattgtttatgaactgatggatactgatcttcatcagataattcgctctaaccaagctttgactgatgatcattgccagtacttcttatatcagctcttacgtggcttaaaatatgtacactct

SEQ ID NO: 161 - RNA, 327 nt, Spinacia oleracea
  source 1..327: mol_type = genomic RNA
tacggcattgtttgctgtgcacgaaattcagagacacaagaagaggtcgccattaagaagattgcagatgcttttaacaatagaattgatgctaaaaggactttgcgagaaatcaaacttctgtgtcacatggaccatgacaatgttatcaagataaaggacatcatacgaccacctgaaagagagaagttcaatgatgtctacattgtttatgaattaatggatactgatcttcatcaaattactcgctctaaccaggaactcacagaagatcattgccagtacttcttatatcaaatcctacgcgggctaaaatatatacactcg

SEQ ID NO: 162 - RNA, 327 nt, Spinacia oleracea
  source 1..327: mol_type = genomic RNA
tatggtattgtctgtgctgctgtcaattcagagacacgagaagaagttgcaatcaagaaggttagcaatgcatttcataacagaattgatgccaaaagaatactccgcgaaatcaaacttcttcgccacatgaatcatgacaatgttatttccatcagggacctaatacgacctcctcagaaagagaacttcaatgatgtatacatggttcatgaactcatggatactgatcttcaccaaataataagatccaaccagcagttgacagatgatcattgccggtattttctgtatcaaatattacgagggttgaagtacattcactct

SEQ ID NO: 163 - RNA, 328 nt, Spinacia oleracea
  source 1..328: mol_type = genomic RNA
atgatgtgttggattgcttgaaggaagataacattattgggaaaggtggtgctgggattgtttacaaaggaaatatgccaaatggtgatcaagtcgctgttaagcgtcttcctgtaatgagtaggggttcatctcatgatcatgggtttaatgctgagattcagactttagggaagattagacataggcatattgttagattgttagggttttgctccaaccatgagactaatctgttagtgtatgagtacatgcctaatggaagcttaggggaggtgatacacggtaagaaaggcggtcatttgcattgggatacaaggtataagat

SEQ ID NO: 164 - RNA, 354 nt, Spinacia oleracea
  source 1..354: mol_type = genomic RNA
ctccaattccaaattcccccacatttaacttggttaggccaatggttcagaaaatgtccttgaatgatgcaattcaggatagagcaagagttgctgaagctgagagtatgcaggttacatggtgagagtagttccagtgatgaagaggattcctatggaaaacctaaagaagagactattaaagctaagatcacaagggttgctgttcgtactcaagcttctgattccacccttatacaaaacacaggtggacgagagaaaaagtgggaatttagaagaggagttgaacaggatgaacatagagaataagaagctgacaaaaatgttggcatccatgtgtaagaattacagtat

SEQ ID NO: 165 - RNA, 324 nt, Spinacia oleracea
  source 1..324: mol_type = genomic RNA
tcagaccctgattccaacccagaaatcaaaccagaacttgactgttgctccaaaatttgtgaagaacgtcataccagctaccgtgcctaatgtttctgatctgcctatgttaccgatgttcaggcaaaaagcataatacagttagcaagtagaaaaacgatacaagaaatatgtacatctacaacaataacaccaacgttacaatctcaacaattactgtttatgatctacccaacgacaaggccgaaagcataatgaagcttgcaatggaaaagaatgaatcaaaaaacgaagggaaggtggtggattattcatgttttaagt

SEQ ID NO: 166 - RNA, 311 nt, Spinacia oleracea
  source 1..311: mol_type = genomic RNA
ttggattgttccaatttttgataaaatgctggaagagcaaaatacagagaagataacatggacaccctcaaagttgatcgctcgtttgggaaaagaaataaatgacaagagttcataccttttttgggcctacaagaataatattccagtattctgttcaaggagtcagaaaccctagaagggaaatgcattaagactgagagttatacccagctttttaaatcgtttgtctccaccggtttccaagcatcgaatctcggagaagctattgatgttgttaatgaaatgattgacgactggaaaatgagata

SEQ ID NO: 167 - RNA, 354 nt, Spinacia oleracea
  source 1..354: mol_type = genomic RNA
ctgaggatggttttgttagcctgctgactgataatggagatactaaggatgacttgaagctgccaactgatgaagttctgaataaacagatcaaggatggatttgctgaggggaaagactgccaactgatgaaactcttcttaagcagctcaaggaaggatttgcggatggaaaagacctgattgtgagtgttcagaatgccatgggagaagaacaaatttgtggcgtcaaggataagcacggccatgccaaatgtaactttgttggtattgatatcttcaatgggaagaagctcgaggatattgttccatcttctcacaattctgatgttccccatgtcactcgtactgatta

SEQ ID NO: 168 - RNA, 324 nt, Spinacia oleracea
  source 1..324: mol_type = genomic RNA
atcaagaagattggtaatgcattcgacaatatagtagatgcaaaaaggacactgagagagatcaaactacttcgccatatggatcatgaaaatattattgcgattaagcaaacttctgtgtcacatggaccatgacaatgttatcaagataaaggacatcatacgaccacctgaaagagagaagttcaatgatgtctacattgtttatgaattaatgtctgtgctgctgtcaattcagagacacgagaagaagttgcaatcaagaaggttagcaatgcatttcataacagaattgatgccaaaagaatactccgcgaaatcaaa

SEQ ID NO: 169 - RNA, 324 nt, Spinacia oleracea
  source 1..324: mol_type = genomic RNA
ccagagtttgctgttggtgatgaaaaacgtctaatgcaggttctattgaatgttattggtaatgctgtgaagtttacgaaagaaggaagtgtctcagttactgctttacggctactgtcgaggagtacattaaagtaaagtttgagtttgacataagtggaggttatctgacagacactattaggagaacccagaatgattcaaacaaggaagtatttggggttgctttcagttctaaaagatgagaatttaagcagtgaacagaaacttattgttgacacaatgatgaagacagggaatgtcctttctcttttgatcaatgat

SEQ ID NO: 170 - RNA, 300 nt, Spinacia oleracea
  source 1..300: mol_type = genomic RNA
atttgagaggtttagatggtatccaaggacctgtctatgtgggtacgggatgtgtcttcaacagaactgctttgtatggttatgagcctcctatcaaaccaaagcccaagaagaaaggtatcctatcatcatgctttggtggatctcggagaaagaagccattcatgttattagctgtggatacgaggacaagtctgaatggggtaaagagattgggtggatatatggttcagtgacagaagatattttgactggatttaagatgcatgctcgtgggtggatgtcgatctactgcatgcc

SEQ ID NO: 171 - RNA, 308 nt, Spinacia oleracea
  source 1..308: mol_type = genomic RNA
aaccaatgtatttggcgacaagtttgatgcttttgcaagaaacttcgagaaatcttttggaagtaccctcagcactctgactttgataaataactcatctatagacaataaagatgatcacgttaggaataacattgttggcgattgtatttctgatgcaacccaatccatctcctttaacaaaggagattgttccgcaagaagtagtgatgttatagaggattgcagaagatcaataaatggcgctactgggcctcagcgtcgtataaaaatggttgaagaggtagaagatattcagtcagcagact

SEQ ID NO: 172 - RNA, 345 nt, Spinacia oleracea
  source 1..345: mol_type = genomic RNA
ccgtcctcgatgttgtggcaaccaatgtatttggcgacaagtttgatgcttttgcaagaaacttcgagaaatcttttggaagtaccctcagcactctgactttgataaataactcatctatagacaataaagatgatcacgttaggaataacattgttggcgattgtatttctgatgcaacccaatccatctcctttaacaaaggagattgttccgcaagaagtagtgatgttatagaggattgcagaagatcaataaatggcgctactgggcctcagcgtcgtataaaaatggttgaagaggtagaagatattcagtcagcagactgaccaggatgggcaccac

SEQ ID NO: 173 - RNA, 328 nt, Arabidopsis thaliana
  source 1..328: mol_type = genomic RNA
attgctaggtttcaaatctcgatgcaatacattagctgaatgaacatatttgagcccacgcaacaactgatacagaaaaaaccgacaatgatcatcagttaagggttggttagagcgaataatttggtggagatcagtgtccataagctcataaacaatgtaaacatcattgaagttctctctctgcggtggttttattatatcctttacagcaatcacattttcatgatccatatgcttgagaagcttaatctctctcaaagtccttttagcatcgataatattgtcaaaagcattaccaatcttcttgatagctacctcctctcca

SEQ ID NO: 174 - RNA, 322 nt, Arabidopsis thaliana
  source 1..322: mol_type = genomic RNA
aatccatgattcttgtgaccatctcttcttttcttgtatcttctaattcggttttgaatttctccgctctgaatgcagctttcgaaacatccaactctagcgccgctttccccaggttgtttgattcaaattttaatcctagcgccgtctctttgcaccttaactctcttatttgaagaccaatctccacggtctttaggtcgtttgctcgtgtttgctcttttaaaaacctttcgtctgtggtcaaagccattgctgaacttctaggagggacacaagagagttgtagagtctcttcgaaaagggcaagctcattcatcaa

SEQ ID NO: 175 - RNA, 303 nt, Glycine max
  source 1..303: mol_type = genomic RNA
gaacataatctttgccaggaaagagcggttgtcttgttatgatttcaccaagtatgcaaccaacagaccatatatcaatggctgctgtatattctgaacaattaagaagcaattcgggagctcggtaccatctagtgaccacatactcagtcataaagtcagtttcagatgtggttctagcaagaccaaagtccgcaatcttaaggtcacaattggcattcaatagcaattactaggctttagatctcggtgcaaaacatttgctgaatgtacatatttgagccctcgtaacaattgatacag

SEQ ID NO: 176 - RNA, 258 nt, Glycine max
  source 1..258: mol_type = genomic RNA
tacactgactgatctcaggcacaataggacccgagaacttgttgccactaaaatcaatcttagaaagctgttgcaacctcccaatctggggaggtatccgacccgtgaacatgttgccatcaagaatgagcttctgcacgctggagaagttaccaatggagggaggtagaaccccagaaagctggttgttagagagagtaatctgaccaagattaacagcaacagaacccacctcaggaaactctccagagagataat

SEQ ID NO: 177 - RNA, 302 nt, Glycine max
  source 1..302: mol_type = genomic RNA
taagaaatgcagcaagcacataatctcccagtataagagccacacaataccactcccacccaatgtctctacacatagcttgccacaatagccacaaaaaattcccaacattaaaaggatgaaagtgactggcatagtctgtgatgttgtggtcgaccgctgcaagagcaaatatgcaacagcaaaaatcatcaagatgccaaatgccattcggctcataacttgaacttgacaccacaaaatatgcaactttgaattgaataagaccattgacttgggagtccaccaggaggaagattcct

SEQ ID NO: 178 - RNA, 507 nt, Glycine max
  source 1..507: mol_type = genomic RNA
tttaactttttgtgagagttcatttggaagctcttggtcaattgaaatctggtcaaggtctgatctggccaccctatcaatgcattgattgacaagttgactcaattgacaatggaaagcaacagtgaggattgggatgacatcttccacaagagccttccctataaagttaagtagacgtctctggaaaagtgatacaaactctggtatttgaaaaatgtaagaggcatacatcaactccacagcaaagttaatggcaggtctacaagcatcatgggcacacacactgtcaacacaagtagacacttccattggagagggcttgagtttaccagtatatacatagccaaggaatatggggaaggcttcatatccaaccttgccataaggcaacaaatcactcatgttatacttcaatttcccttctttttctgatgaacccttctctctcttgaataattcatggaaaaacttactcctagaggccagaatacatcgatgaacact

SEQ ID NO: 179 - RNA, 350 nt, Glycine max
  source 1..350: mol_type = genomic RNA
acatcaattcctctatcatcggatgcattgagctccaaaatcatattttggtactgtgaaccatacagtttgcgtgccacagcaagaatcgttgatgttttgcccgtgccaggagggccatacaacagaagatggggcaatctgttctcagtggttagcctatcaattgtatcgacaatgtcacggtgagcagcgacgtcgtcgagggactgagggcggtatttttcgacccacggaatggccttgccggcggcgggagggtttccggcgaagacgacacttttgcccttgtcaggactggcggtgtaagtgtggtcgtcgatgtccatgggatgggtcgtctgcgccat

SEQ ID NO: 180 - RNA, 366 nt, Glycine max
  source 1..366: mol_type = genomic RNA
cagtagcaggctgagcaaagactccctctgcatgctccagttcaaggttgcattgcgaggtaaaaaatcatcctcaaaaacaacgacaaggtactgaaagattaacatgttcctcaagggctccatgcttgctgcttcaaatttggtctctgctaaaacttcagccagcccttcaatcaagacttgaaagctagaggggtcaagagatccaccagactgtttatcaccagcttcactaacagcagtggccttatccacaaaatcgataatgggagaccatgcctctttaaccacaaccaattttgatggagagttagatgatgatcgggaatcatccgtgccagataaatacacgcgagggaagtg

SEQ ID NO: 181 - RNA, 328 nt, Glycine max
  source 1..328: mol_type = genomic RNA
aacaacccaggctttttgtgcttgggtttaagaggaggttcataaccatataaagctgtcctattgaagacacatccagtacccacataaacaggaccttgaatgccatccaaacctctcaagtttatatcaaagaaaactgtattacgattggcatatcgatcattcctatcaataccatcaaacctctgtggaaactggacatagcaaacatgtttaccaaggtttggatccatcataaagcacatagcttccctcaaggccttgctgttgtttatgtagtgatcacaatcaagattcaataagaaaggtccattggtaaggactg

SEQ ID NO: 182 - RNA, 400 nt, Glycine max
  source 1..400: mol_type = genomic RNA
gaaaagaccatcggtggtgaaaccggagcacttgtcaagcttgagagagtgaagcacgtggccgcggtcacgagcgagattccgaagatcggaatctttgacaatcatacggcggaagtggagactcttgaggcaatcgaagtactgagaaatctccttgacccatggggtgacgtggcctccccaatcctcgggtatcaagttgaacattgctgctcggggcttgcccttgagcttgagcgactcaaggtgcgggaagcggcggcggaggcgcgccggcgtggtggtgtagcagagcgcgatggtgacgtgcttccgagtgagggagtcgagttcgtaccagcgtcggcagacctgtgagacggcgtcgcggtccttggggtcgtcgatgtaagggatg

SEQ ID NO: 183 - RNA, 365 nt, Glycine max
  source 1..365: mol_type = genomic RNA
gatccatttgaggcaccacttctatcaacacttatttgttcatgattatttgatgtcttttcacctatgcaacctctcaattggctatgatctttgaagactatgtctaaagaatcaaatatagggtcagcaggctcatatagagaagcaaaagcccacaaaagctgagcaagttcttgttcttggaaagtgtgaattatgtctgaagccctttttgaaaactctgagaaaagatcaggagcagaatgctgcattgcagcaaaggcacctgcaatattagcaatgttttgagagttgaattccccaaccttggtcagtgcaacctctgcaattctatccatttcagaaagataaagcagctcgcc

SEQ ID NO: 184 - RNA, 279 nt, Glycine max
  source 1..279: mol_type = genomic RNA
aggcatacgatgggttctgaaggaaaacctatgcttcctgttctagatttcaccatagaagatttgaagccagggaccaattcctggttatcaacatgcacaagtgtgcgccaagcatttgaggagaatggttactttgttgctgtgtatgataaagcttctattgagctacaaaatggtgtatttggctccatgaaggagctgttcgatctcccaacagaaaccaaacgaagaaacatatttgagggcatgcccttgaaaggctacgttggacagcac

SEQ ID NO: 185 - RNA, 201 nt, Glycine max
  source 1..201: mol_type = genomic RNA
aatccttcaaacgcttcacatcagataacttcactggcttcactatgaaatcctctgcaccctcctccaaacatctgtctatgcgtggcaaaacgttttcagatgacataataaccacaggcgtttccttgaaagtagatgattccttgattctcttgagtaattcataaccggtcattccgggcatacaatagtctgtga

SEQ ID NO: 186 - RNA, 443 nt, Glycine max
  source 1..443: mol_type = genomic RNA
tgaccacagataagcttgcaatggaaatgacaaattttaccatcttggaatgacccgaatcctcaagcatcaaagaaaaggcagagcaaaatgcaaccatcatggccaagatagaacatatgagtgtgaaaaggccaaacatcaacttgagtggcaaggatttaaggaaatctctttcagcataacgagatgtgagaattccaataaaagtcatcacagcagtggaggaagaaaagagtgagatagcatcagccaccatataagttgtgaacattcttttgtgcaaaaacataggaataccagtattttgatcatttcctccaggaagagtgaaggctgctgcaaacacaatggttattataagagtccccactattgtgaaggatgttgccgtctcctttgcccatttctctgcgtccttcaacagttcttcatgtttctta

SEQ ID NO: 187 - RNA, 296 nt, Glycine max
  source 1..296: mol_type = genomic RNA
ccactgtcatcatcatcgctatagcagtttacaccttgttgagacctgatcttctcatagactttgcacaaaacccacttgctccaactttgatcatgttttcttcttctcctagcacttcgatatgaggcagtgtcaaacccagaggaacatatataatattcttgcattacccaactggtttcagtaccttgtggagcttcacctatgtggaacaccaagtacttcttcattcccactttcttatcaaaggttgacaatataggttccgtgacacctgtgtccttccaataccc

SEQ ID NO: 188 - RNA, 360 nt, Glycine max
  source 1..360: mol_type = genomic RNA
gctccaatagcaattttccgcctaatggcccagttcagcttgatcccagctttcttctgatcatgtagaacatcctctaaacttccatatttcatgtactcataaaccaagagcctctcttcccctaccttgcagtatcccaacaggggaacaaggttcctgtgcttgatcttcccaatggtctccatttcagcagtgaattccctgtccccttggccgctgacatgaatcagcttcttgatagccacaacacttccatccttcaactgagccttgtaaacatccccaaacccaccagagccaatgagactatcattgtgaaagccattggtagcgtcaagaaggtccgcaaaagtaagc

SEQ ID NO: 189 - RNA, 310 nt, Glycine max
  source 1..310: mol_type = genomic RNA
tcccaacatccaaaactggtgtctttccgtacaagttcttgagctctagaggaccaagttttggacgacgaagtccaaattgagctgtgtctccaagcatgagatgtgacattagaagcaaaaattgatccacaaaacgtatggggaaccacttgttgggagttttcttgttcgcagctgtcagacactgcctttaaaaccttggcagctctgagttgagaatccttcgagacaccgattgtacttatctctatgaatttagtcattttctttgctacctctttatctttgacaatagcccaccctatgc

SEQ ID NO: 190 - RNA, 399 nt, Glycine max
  source 1..399: mol_type = genomic RNA
gggtcagagatggctatggaaatggacggtcgtaattgaaaccgaagaagaagagtcatgctttgaggaaaaccatgattacaaggtactaaccatatgttcccctgcatcaactggactaccctattacttggaaagtccttgcctcatcaccaatgaccagatcaggcaaagacttgtcaacatcaacttcaagaccaaagcctttatatatacacaaacacttggcagtgcaagaagcttccctcaaccagtttcttcttgtgtgcgaatcaggcccatttctctatcaagctctgcagccttgttcttcaaaaataattccctagttttaacacttaataaatcaggaataatatgtaccagcatgagggcagttgcacacgataccacagcagt

SEQ ID NO: 191 - RNA, 367 nt, Glycine max
  source 1..367: mol_type = genomic RNA
gtgcctaatgggtttgatggattggtgatgagcaagccctttactcttatattatcctctttggccttctcatacgcatcttccaatgcttgctttgtcaacttgaaattgtttgagctatcgcacataactggaacaagtttaattcctgttctccacctcaaatcccggtcaaaacctggataataaggaatgggcaccaaaaatgcgtcaccagggtctgccaaacaaaaggtagtgacttcgtgtgctccagttgctccaccgctcatgacaatacggtcaggatcaaacgtgactctgtttcctcttgttctacccatgaatttagccacagcatttctgaactcgggcagaccatgataat

SEQ ID NO: 192 - RNA, 397 nt, Glycine max
  source 1..397: mol_type = genomic RNA
ccatttccagggtagaagcaacattttcacagcaaaactggaaactattactcttattctgttgtgtatccatcctgttgcccaaagttctctcattcctgcatcaaccaaaggaaagccagtcctaccttgtctccaagtcttaaagatatcaggatcaggattccaaggaaaaaactttaagtgccccagaagtgcaagtgcattgcttgccttctctaattcatcttcaaacagcaatgtatcactagggcacctcgatgcatcacctggaattatccttttaggtgggagaagaggtgactcagggtcataaggcatgctaagacatctttcccaaaaagcagcaaaagttgtgaatggtcggccatgtgcatcattcacatcccatggttca

SEQ ID NO: 193 - RNA, 376 nt, Glycine max
  source 1..376: mol_type = genomic RNA
gccagtttctccagttccacggcaaaatccttcattaccttcctgtaatcttcatcaagatcagggatctctgagatgttagagacaggaagatggcgcaaaaagaaagtgctttcccagtccaaatcattgatttcagattgagcagactccaagcctttgcttgctaccatttcttggaacctttgctccatgctgctctgcaagtttctccagttccagagcaaatttcttcattgtcttcctgtaatcttgatcaagatctgcgttgtctgaaacgttggagagtggaagatggcgcaggaaaaaggtgctttcccagtccaagtcattgatttctgactgaactgactcaagacctttgctggtcaccatt

SEQ ID NO: 194 - RNA, 390 nt, Glycine max
  source 1..390: mol_type = genomic RNA
tgaccctgtcccaatgttctccttcaaaataagttcactccatggaatatccagatcctccttgtcaagatcaacttcatgacttgggtaatccattattgactggtttgcttcattgaaagactcatattcctctgcaccaggaatcaaagcaatcagatggctgttcccacccgcataatttgcacccagagcttcagtaattcatcccctttgccacgtcataagccataccaagcctacgcctctcatccaatacttctttggcgccagatctatgcaaaagcctatataagctacccctagataaatattccgtgacaattgataagttaggaggctgagtgactgcccccataaacaaaacaatgtttggatgccgtaaacgtt

SEQ ID NO: 195 - RNA, 421 nt, Glycine max
  source 1..421: mol_type = genomic RNA
catgttgttcaaagtcaaagttgaagggagtcatgcacactggttcatcactgatgtcatgcagagatgtcaagtacgggtgcgccagtgcatcttcaacagtaatcctttttctaggatcaaaagttaacattttttcaacaagatctatagcttcaggatggacatgtggaaacttttcctggaaagattgacggcggtaaaggggtagttgcctaatgtatctcttagcattttcattcaggaaccccaaatcagcctctgatggggtgccaatcagctccataagtagacgcaactgatgcacatgatctctgccaggaaacaaaggcttgcgatccatcagttccatgaaaatacaaccaacagaccatacatcaattgctgcagtgtaatcagaagagttcaacagaagctctgg

SEQ ID NO: 196 - RNA, 243 nt, Glycine max
  source 1..243: mol_type = genomic RNA
tggggctctagcctggcgatgaagcggtgaacgttcaatgctgtactcagacccagtactttgcgttgaatgtcttctacggttatcagcagaacctactccatggcttctactgccactcctactgcttacattgtcatgacgagctggagattcgataaaattcttgggatcatcttccaaatgcagtgatccttttccggattgatcctctgaattagttctaggtctggtaggaggtag

SEQ ID NO: 197 - RNA, 159 nt, Glycine max
  source 1..159: mol_type = genomic RNA
ctggactttgtcatcgttagattggcccctaatagcttgtggtctctcattaggcgttcccggcacttgtcctgggacaccctgtctctcctccctcactttgttgaaaatgtgagtaaagccatctgctgatgatgggttattcacatcccagtcacc

SEQ ID NO: 198 - RNA, 299 nt, Glycine max
  source 1..299: mol_type = genomic RNA
tacaagcttcttggcctcgtctatgaccatccacaatcacagccaaaacaaaagcagccattgcgcgttgctctggatatgcttccatgctgtcaagaaacttaataaaatagatatgaccaccatccttcactagatcaacctgacatgacttatcaagtgcaagaatctttgtccatatgaatacaaggatctgtcgtagttctggtgttgttgtttgtaacagctttagaacatatgggaatattccaacagataatgccagatctacagcccatggccccatatcaaggaacctt

SEQ ID NO: 199 - RNA, 297 nt, Glycine max
  source 1..297: mol_type = genomic RNA
tcatcctggcgtaaatcctcatgtcccttcaataagaaggcataatcatcaccatcactcccatgaatggttaacttcctgggtcgttgcttggatgttatgacaacaagttggcgtgcaaatgatgcaattgtaaccactggtgcatcagcacggtatgacccgggaacagcaagctccaaattgcggcattctagcaattctggagaaacagactccaagtctaaagttgtaagactttgaagttgcttatctatctttctgaaaacatgataataaatatcccaagcctgggtc

SEQ ID NO: 200 - RNA, 360 nt, Glycine max
  source 1..360: mol_type = genomic RNA
cacagacaccacaacagccttacagatacccactaggcgacgacaaagggtgtcatatatagcactattggctgaagaatgcgcaaaacttccaccatggaaaaatatgatgacaggaacaacctcagagctcacaggcttctcaaggtcaagaatgttcaccgaacgttcttctcctggtcattgtttggcaagaagtagaagccaatagtggcttccttgagaaaaagaagcttgacctgttgtccacaatcctcaagaccttttgcatatgccaattgccaatcctgcagaagatccaaaccagccacacaaacaagacttttagggaatttgagtccttcaatgcttctccccctt

SEQ ID NO: 201 - RNA, 350 nt, Glycine max
  source 1..350: mol_type = genomic RNA
cagttaagcccggacagaaaactggaatgttgttcttgtatgcccagtaaaggtaagagctttcattttttatttcttttcccaatcgagctattaacttagatggtgtccacaacacattctcattattttgttcccccaacatctgatcaaagataggaataatccagtcctcaaatttgcaatagttgtcattcgggaccaataaattaccaatccgattcaacccttttgagcgcagatatgctccaggcaaagagaaatctcctttatatgttggtgccaggcactttataagatcttcttcaatgccacctgtagttgtaacaactacatcaaccatgcgatgc

SEQ ID NO: 202 - RNA, 339 nt, Glycine max
  source 1..339: mol_type = genomic RNA
gcaacccgcttatgttgttttggtaaaggattgtgagaagggtggagtcggtgtgggcggccagacccatggcccgatccggatccggacaagtcgggtaagagttcaattgcaaggctgcgcatgtctttttgaattggcctttggacccggcccatttcaggtcttcctttgtaatacccagagaatccaacatcagccacatcagctttcccactagctttttcatggcttcatcatatcgcttgacgatatcacagtatttgtggtaatcttggggccagagttgacgaaaatgctcaagaggggatccaacaattgtgaatccctcagaccaca

SEQ ID NO: 203 - RNA, 300 nt, Glycine max
  source 1..300: mol_type = genomic RNA
acgttcaccttcatgctccttttcaaattccttctttctttccttttgtagtttttttcttctttcacttatcatttgcctaagttcatgcacatctttcgtgataagatccaaaggttgtaagaatgaaaaatcttcttccaggtctgcatcacaattactcaagttggcttccatctcaaagtactgtagacccttctgaataaatgtaacaagagcacctgacggcaccaaatttccatcaatggagcatttgttaataccagcttcataaccaaaagtaaaagcagcatgcgtgaa

SEQ ID NO: 204 - RNA, 362 nt, Glycine max
  source 1..362: mol_type = genomic RNA
ttaccctcagcaaacccatcctttatctgagtaagcaaactctcatcagtaggaagcttcaggtcatccttggtgttcccattttcagtaagcaagctaagaaatccatcctcagcaatgtcaatcaactggtaatcagtacgattcacatggggaacatcacagttgtgagaagagggcacaatatcttcaagcttcttggcagtgaaaatatcaatcccaacaaagtgacacttggcatgaccgtgcttaccagtcttggaagtagaaacttcaacaaccttgcagggacgacccttgatgacgatgtaaccgtttttgcggatggttccggcttgttgaggataggtcttggaggctcc

SEQ ID NO: 205 - RNA, 243 nt, Glycine max
  source 1..243: mol_type = genomic RNA
aagtatgaatccatgtgaaactatgtgggctaggagcttagagtagtagctagtgcgaatacaaaagccatggcaaaacaatattacagggtataagccaggcacggttggtgtaaaaataagcaatggttttggaggtgaggaagaagcagtggatgtttcaacattgaattgcttccaatgaatatctcccttctgaaaaacatccgtggtggccaacgctggttgagctctctgcgccat

SEQ ID NO: 206 - RNA, 287 nt, Glycine max
  source 1..287: mol_type = genomic RNA
cacctgaagggtcggaacatacatataacttcgggggagcttttaaacagaagggccacacagacacagacagcggctattgccaccattgagagcattgcaggtctgtataccaaagacctgcctactgcagttctgcaagaaattctcttgtcacaaagcttacagtgcccttggccacgtattacttcagcctttccaatctcaaaggtagtggataactcattttgcttctcgtttgtattgctctccgtcgtattgcttggaatctcaaccaccacatgagc

SEQ ID NO: 207 - RNA, 307 nt, Lactuca sativa
  source 1..307: mol_type = genomic RNA
aaacaatggtggcttgttcttggcttgtggctgtgttttctttcatatcaaaatgaaaatcataggttgaactcccttccacgtgttgatatgatcttttactgttttctgatgtttcattgattactctgagtgtcatcaaggtgctctgaaatgatctttcgaagttgcttacaaagtaatcaaatttgttgccaaaaacatgagacaatgattctttaactgccaaagtgcatatctgggagagatgatctatagacatcttttctccagttgcttctttcttttccaacacctgagcaacaaa

SEQ ID NO: 208 - RNA, 344 nt, Lactuca sativa
  source 1..344: mol_type = genomic RNA
gtggtgcccatcctggtcaaacaatggtggcttgttcttggcttgtggctgtgttttctttcatatcaaaatgaaaatcataggttgaactcccttccacgtgttgatatgatcttttactgttttctgatgtttcattgattactctgagtgtcatcaaggtgctctgaaatgatctttcgaagttgcttacaaagtaatcaaatttgttgccaaaaacatgagacaatgattctttaactgccaaagtgcatatctgggagagatgatctatagacatcttttctccagttgcttctttcttttccaacacctgagcaacaaagccacaacatcgaggacgg

SEQ ID NO: 209 - RNA, 348 nt, Nicotiana benthamiana
  source 1..348: mol_type = genomic RNA
tcagatttgcatcaattagccatggcctttgttcccctggaggcaccttattattgtgatgatagtacgaaatgatatatggagccaaagtatggtgatgatgaacatagagcaaatcgaacagtacactgttcctgagttgctcatcttccgataatgtgtcttcaagctttttcgtttcagcaagtaacttcttctcgtcgatgaacggcaattttacaacagcctgccatgcaaaacgctttccattcatgtcaagttcaaaatctgttgggtaaaaatcagaaattggcgatgatgaatccatcattagcatcctgtacttttcaggaagagcatttgcacttg

SEQ ID NO: 210 - RNA, 348 nt, Nicotiana benthamiana
  source 1..348: mol_type = genomic RNA
acataatccttccacatacttctggactacatcttgtctgacttcatcaataccctctggagttgatacaccaaatttctcggaatagtacctttccttgtatccaggctcccctaacttcaccttgtcctctgccggaggctgtggctgagcattaacctcatttcttgctctttcattattttctattgactgctttatcctagctctcttttgaaagatttggtcttcataaactgctgccgcctgaataaaatgttctactctatccaaaaaaacctcacccgcatctgtaagataaccacccattgttgtaaactctcttctatagatgcacatcagcagatt

SEQ ID NO: 211 - RNA, 300 nt, Nicotiana benthamiana
  source 1..300: mol_type = genomic RNA
caaaacacttctcctgctgacctggtgatgtaatcacctccctcaatattgaaaaatggacttcatgagtagcaagagacagcataatcagatctgcatctaggccataaagacaatgacgggtatttgggtcgaagccaggaaggttacaactcttccctcaaccttttttcttcagcttcagcttcagcggcatctttagcggctctaaaacgcctggttcgctgctggttcatcttagctctaggagccactccatcaatagccatatagaggagttttcttggacggactaatgag

SEQ ID NO: 212 - RNA, 347 nt, Solanum lycopersicum
  source 1..347: mol_type = genomic RNA
atagagcaaatcgaacattacactgttccttagttgctccgtgtccgttaacgtgtcttcaagcttctttgtttcagcaagtaactttttctcatcgatgaacggcaattttacaacagcctgccatgcaaaacgcttcccattcatgtcaagttcgaaatctgttgggtaaaaatcagaaattggtgatgaaggatccatcatcagcatcctgtatttttcaggaagagcttttgcacttgcagctggtagaacacccattagctgatcgaagggcttaaatggttcccctgggaaaaaagtaatttccaaatctgctagacctttgagatcagaagcaaatggag

SEQ ID NO: 213 - RNA, 308 nt, Solanum lycopersicum
  source 1..308: mol_type = genomic RNA
tctatacaggttttaagtagctcagcatgttttgagtcttctacttgatttttgaacttttcagttttgaaggacgttttagagaaaccaaatgatagtttgaccctctccaaaaggttagcatcagaactgagagccagctgcctttctttaagttgcaacttcctcattgtaagaccaatctcaaatgtcttaaggtcattcgacctagcttgttctgtgagagatctttccaaagtcctatgcacagattggctaatccctgtattggataaaatcatgcttgaagaagcagaagccaactgttg

SEQ ID NO: 214 - RNA, 345 nt, Solanum lycopersicum
  source 1..345: mol_type = genomic RNA
gtggtgcccatcctggtctctatacaggttttaagtagctcagcatgttttgagtcttctacttgatttttgaacttttcagttttgaaggacgttttagagaaaccaaatgatagtttgaccctctccaaaaggttagcatcagaactgagagccagctgcctttctttaagttgcaacttcctcattgtaagaccaatctcaaatgtcttaaggtcattcgacctagcttgttctgtgagagatctttccaaagtcctatgcacagattggctaatccctgtattggataaaatcatgcttgaagaagcagaagccaactgttggccacaacatcgaggacgg

SEQ ID NO: 215 - RNA, 328 nt, Spinacia oleracea
  source 1..328: mol_type = genomic RNA
taatctgaccatctattgctgtctcaatcttctttttcattggacaattgggaaacgtgcatttgtaataacttcttggattttcactcccttttacctgtttttgtccatattttctccaattatacccatcttctgatcttctctgttctcttatgtattgagctggttggtagggagggttttggtctgtctcatactttacaacgcttccttggttgtaattctcaccaggaaactctgatttcaccatagctgtaattgagttcgaagatccatcattcttttgatcgaaattccatgattgttgttggggttgtgctggttg

SEQ ID NO: 216 - RNA, 328 nt, Spinacia oleracea
  source 1..328: mol_type = genomic RNA
atatttcctccaacaatacccatcttcagctcttttctgtgctcgattcatctgaactggttgagcattgtaccctctttgcatattagtaccaacatctgtttttgttgtgttcatttcatatggcatggtttgattaacagtagtaaactcggattttgaattgatttgatctttgttgttaatcatctcattttgcttcttcggttcatccaaattccacccttcaattgagttgtttgaagaagcatggaaagtaaaattgttgtttcttccttcatccttattagcacctagttgagtccaattgaatccctcaccaccaaag

SEQ ID NO: 217 - RNA, 324 nt, Spinacia oleracea
  source 1..324: mol_type = genomic RNA
agattttaccggaaaagtcttcggaaaactcttggtggtggtggtgacaactcttattatgatgagaagacgaagacgttgaaaggggaggtttcccagaagcagcaaacccagacgacgatgtaggaggtggaacaaaaacagatccaccgaatagctttctgttagacacgcttccttctccggtaatagtagacaaaaacgacgatgagttcgccgatgaagatatactaaaggacgccgaattcgaaatcgacgcacatggttttgtattcgggttcggttttgtgaaatccaaagtcatcgggtggttgtgcgactgtc

SEQ ID NO: 218 - RNA, 313 nt, Spinacia oleracea
  source 1..313: mol_type = genomic RNA
cctctaatattgtcacatctgaagtagaaatttcacactccatggaaggtgatgatggagagatatccatgggctcaggatcaggacaaactctattctcttcatatttgatggcaggcttctcctctcgctcaacaactgtttccatatcagaaggcacatcttgtaacttgggtttttcctcctcctctttgatagatttctcttccctatctttcccaacattttcttccttctctttcactgctttttcttccctctctttcccagatttttcatcctttacttttccagctttttcctctttctcttt

SEQ ID NO: 219 - RNA, 276 nt, Spinacia oleracea
  source 1..276: mol_type = genomic RNA
caagagtgagttcaagatcatccatgcctacatcatgaatcctttccccctcccaaggggtaacctgcatactctcagcttcagcaactcttgctctatcctgaattgcatcattcaaggacattttctgaaccattggcctaaccaagttaaatgtgggggaatttggaattggaggtctggaaaacctttggaagctaatccactgtccggaatcaatagtagagcagtcagattcatcacattcaggtatggtaggcggggtaaagttcctcc

SEQ ID NO: 220 - RNA, 328 nt, Spinacia oleracea
  source 1..328: mol_type = genomic RNA
attttgacgtaaattttgggaagctgatgttctagatgaagttactggtgccgctctggaatctttgtttccattgttatttctcgggagttgactttcaacaatatcataatctttattcctggggacataattgatagcctggtggtttttccacatcatgggagaaggcggttttctggagacagcatcattcttaaatctctgagactcatcaatctcgtatttgggtttattttgctgtgaagcttggccagaaactcgtaaatggaatggattaagttccgcaaaaagattctttgattcatcattattaccttgattatag

SEQ ID NO: 221 - RNA, 246 nt, Spinacia oleracea
  source 1..246: mol_type = genomic RNA
ttacccattggcggaaccttgacctttgctttgatgtatcgcttctagacactcctttgctctatggaccttcgattggatgtagaagctcccttgcttggcattcctctcaaacaaaacatcatattttttcaaaatatcttcccaggccgcattctcaggaacgccaagaatctgccttgcttcttgttctgtgatggctttacttgctctgcgtacggtattgttcattgcttcctgagctgc

SEQ ID NO: 222 - RNA, 288 nt, Spinacia oleracea
  source 1..288: mol_type = genomic RNA
taacctttatttcgatacacttcttccagatgttcctttgctctatgtaccttcgattggaggtaaaagctcccttgtttagcattcctctcaaataaaacatcatattttcttaaaatttcttcccaagcggctttctcggggactccaagtatctgtcttgcttcttgttctgtaagagcctgacttgctctacgaatggtgttttgcattgcttcttgcgcagcaccagtttttgaggcattgacaatagcttgacgataagcttgaccaaccgcccgaattaac

SEQ ID NO: 223 - RNA, 323 nt, Spinacia oleracea
  source 1..323: mol_type = genomic RNA
tggttgcgccttggaagactttagctttcttgatttcttgtcggaagttttcttaacctcacgtgattcaacattttttgcgtcttttgctgcagatacatcttcatgacactccttttcagagttttcctctagagtaccagttggtgtaagcttattatcatttgttctctcaaacatcaaaatctgattttccctagggctcacagatgattcagaaaacttgggtggtacagagtcagcacttagcacactcatccccaaatccacaatcaaagtaggttgtgctgtttttgagtttctctctaccacctggcctgata

SEQ ID NO: 224 - RNA, 327 nt, Spinacia oleracea
  source 1..327: mol_type = genomic RNA
cctgaccctcttgttgtgctggtggttggtggttgtgctctccttcatacgttgccactaacattgactggtcttctagacttctttgtacctttttcttaacggggcaaccaggagcaaaggagcatttgaagtaagctctagggcaagggttatctcttgtaactttttggccatattttctccattggtatccatccttaagaataagggtggaatcagaagcttgagtacgaacagcaacccttgtgatcttagctttaatagtctcttctttaggttttccataggaatcctcttcatcactggaactactctcaccattat

SEQ ID NO: 225 - RNA, 326 nt, Spinacia oleracea
  source 1..326: mol_type = genomic RNA
aatcaacattttattagtgatcttaggcttgctgctaagaagagttactcttttgaatgaatcatcgtagctaatacaactctgttctgtgttcccctgtattccacactccacagcttcaataaacttcctcttccttggattctgatcttcattttcgcctttcgacgtcggactaatcaaaccaatcaactgattttgcagcatactgtaattcttacacatggatgccaacatttttgtcagcttcttattctctatgttcatcctgttcaactcctcttctaaattcccactttttctctcgtccacctgtgttttgtcat

SEQ ID NO: 226 - RNA, 326 nt, Spinacia oleracea
  source 1..326: mol_type = genomic RNA
cttcaaagctacaagctaggtccatgacttcttttgccttgtcagtcggcaagtcattaaagaccaagacttgccctccatagaagatggtcatttggggagtcggaggctcggggttagcacgcttgttcacgcttgaattaaacccagaaagctgaggaaagagattgattgttttcccagcacaaacatttgaatcttcagctgcatgtttcccactcacagggaacaaattcatcgttgaagttggtcctcgggactcgatgtttccagtaacaccttgagcgcgtttctccttaacatactggctgagaaggttacaagtc

SEQ ID NO: 227 - RNA, 327 nt, Spinacia oleracea
  source 1..327: mol_type = genomic RNA
aggtagcttggattcgatgctttcgcttttcgagaaattggcgtaacgatttcttcatggccgataaaccattttgcgtttgagtacataattgttgagattgtaacgttggtgttattgttgtagatgtacatatttcttgtatcgtttttctacttgctaactgtattatgctttttgcctgaacatcggtaacatcgcaaacgcagatagttccattataaaatatcgtcctttgttgttcttgcttttttggattatctccattaacagatcccatggagatcgtagaagctgttgaagagatcgacgtggaagatggtgata

SEQ ID NO: 228 - RNA, 328 nt, Spinacia oleracea
  source 1..328: mol_type = genomic RNA
tgttctatgtttgtatatggcgacaccaacctctctttgcgcttctcaaagaacctctgcaatgacttcttacgtttaataggcatatcagaatcatctccatcatcatcattaagtcgtccgaggatattcttagtagttgcttgggatgacttaaaacatgaataatccaccaccttcccttcgttttttgattcattcttttccattgcaagcttcattatgctttcggccttgtcgttgggtagatcataaacagaaactgacccattgtagaatatggtcattggggcagtttcttctgctttctccatgctatcttccccag

SEQ ID NO: 229 - RNA, 326 nt, Spinacia oleracea
  source 1..326: mol_type = genomic RNA
tgaagaattcaacaataactctggtgcccgataccatctagtcacaacatattcagtcataaaatcagtctccgatgttactcgagctagtccaaaatcacagatttttaaatcacagttggcattaagcaagagattgctaggctttagatccctgtgtagaacatttgcagagtgaatgtacttcaatccacgtagtatctggtagagaaaatactgacagtgctcttctgataatccttggtttgaacggataatttgatgaagatcagtgtccatcagctcatatgcaatatatacatcattaaatgcctcccttcgaggag

SEQ ID NO: 230 - RNA, 328 nt, Spinacia oleracea
  source 1..328: mol_type = genomic RNA
cttccaatttatcagcttcaccttccgctataggataaacctgttcacccaacttcaaattgagtggtctagcttcatcattcaactcctcctcgggaacaaccacacccctccttctcttctccatttcccttttccctttctcattagttttattaatttctttagaacagacattggtgttagccacaagttctggtccaagaggaaaaagctgcttacttctacaatctaaaggtgtagaattcaatggactggcagtaaaaagatcaccatcccatttccaatcattcaaatcccattctaaagtcctttttcctaccgtctt

SEQ ID NO: 231 - RNA, 328 nt, Spinacia oleracea
  source 1..328: mol_type = genomic RNA
ttgaattcattatccaaatcatcaacatcctcctcttcatcatcgccctgcactcgaagactccctttctgtctcttgtatcttgtcttgcattgggggcaacactggtttccatcttttctctcatactcataacaaggcctgcaaactggaaaagcacattcattgcaagcgacaaagacaccaccattcgaagtaactcccacggtatccccacagatctgacagatttgtccatccaaattcttcaaagatttggacccactatcagtactatcatggcgaattctaacaagttcgttccttttgtaagatccagccaccattc

SEQ ID NO: 232 - RNA, 328 nt, Spinacia oleracea
  source 1..328: mol_type = genomic RNA
acaaatacttctgatttaccaaaccgtttctctaagcttatttgtgacattagtagtgacttttcatcgtcaaatccagcaccttcaactccttcctcgatatcttccagattaaatattggcacagtagggtcagcatgttttgatttcttcttggagtctttcttgcttgatttcttccgagatccaccaaagcatgatgataggatacctttcttcttgggctttggtttgataggaggctcataaccatacaaagcagttctgttgaagacacatcccgtacccacatagacaggtccttggataccatctaaacctctcaaat

SEQ ID NO: 233 - RNA, 328 nt, Spinacia oleracea
  source 1..328: mol_type = genomic RNA
ttgaattcattatccaaatcatcaacatcttcctcttcatcatctccctccacgcgaagactccctttctgtctcttgtatcttgtcttgcactgggggcaacactggtttccatcttttctctcatactcataacaaggcctgcaaactggaaaagcacattcattgcaagcgacaaagacaccgccattcgaagtaactcccactgtatccccacagatctgacagatttgtccatccaaattcttcaaagatttggacccactatcagtactatcatggcgaatcctaacaagttcattccttttgtaagatccagccaccattc

SEQ ID NO: 234 - RNA, 328 nt, Spinacia oleracea
  source 1..328: mol_type = genomic RNA
attataagcaaatcagcaacaagaaagtctgcacatgttctgaggagctcactatgtcttgtctcttccaattgattcttaaacttctcgactttgaaggatgttcttgaaattcctagagacaatttacatctctccaacaaattcaaatcagaattaagagccagttgagtttccttcagccgcaactttttcataattagacctatttcaacgctctttaggtcatttgcacgcgtctgctcaacaacagatttctcaatggtactgagcactgaatagttgctcaagtgatttcccatatttgaaacaacaatttgtgagctta

SEQ ID NO: 235 - RNA, 326 nt, Spinacia oleracea
  source 1..326: mol_type = genomic RNA
aatacaatattaccatattggtctaaatcttcgttataagaaccaccggagaactcttgcaatctcgtcgctgtacgaagaaattttccaagatgaacaagatcagtatctccaattttcacagagactaaaggacaatttctggcaagaagttcaagatcttcaactgtgattttctccataaaagtcatatagaagttcagagtttcaagaactatattgtgtagtgccagctggtgaaaccattcaccattagtctcctcgacaccactgtcttccagaaacagtgttctcaggtttctgcaagagctagtgatacgcaagag

SEQ ID NO: 236 - RNA, 328 nt, Spinacia oleracea
  source 1..328: mol_type = genomic RNA
ttcaccatctttagtttgaacttccaaaccattaacttgattctgatgaagtattgttatgaagtttttgtcagtgtgaccaactgcaccaagattaagttcatccttgagtggtaccctgtactttgctatacgaagaaggtaagtggtcgagttgagatgagagttaaggtatttctccaccccgtaagtctcaaataccatcctcattaccaattgttctagctccgatactgtctttgcatattcatggatggtttcacaaaaatgatcgttcctagtaggccacaagagattagtgaaagattggatgttatgaagcgttgtt

SEQ ID NO: 237 - RNA, 327 nt, Spinacia oleracea
  source 1..327: mol_type = genomic RNA
cattcatggctcttatatcttgaactacgtcaacaattaaaccgggattccgtattgaatgaaaatacaacatgtctccaagtgatccatctgttaaacctgggcagaatactggaatattattcttgtaggcccaaaaaaggtatgaactcttgtcatttatttcttttcccaaacgagcgatcaactttgagggtgtccatgttatcttctctgtattttgctcttccagcattttatcaaaaattggaacaatccaatcctcaaatttgcagtagttgtcatttgggaccaataagttaccaatgcgattcaatcctttcgagc

SEQ ID NO: 238 - RNA, 281 nt, Spinacia oleracea
  source 1..281: mol_type = genomic RNA
tatctcattttccagtcgtcaatcatttcattaacaacatcaatagcttctccgagattcgatgcttggaaaccggtggagacaaacgatttaaaaagctgggtataactctcagtcttaatgcatttcccttctagggtttctgactccttgaacaccacagagcgaaccgacgccaaaatatcactatcacccatatttctacacctgaaattcctttgtagtagaacgaatgagataggttggataggatcatatgatcggaattgtaacctcatctg

SEQ ID NO: 239 - RNA, 326 nt, Spinacia oleracea
  source 1..326: mol_type = genomic RNA
atgtccttgagggcattaatttgttcttctcccatggcactcatcacgctcacaatcaggtctttcccctcagcaaatccatccttgatctgtttattcagaacttcatcagttggcagcttcaagtcatccttagtatctccattatcagtcagcaggctaacaaaaccatcctcagtaatgtcaaggagctggtaatcagtacgggtgacatggggaacatcacaattgtgagaagatggaacaatatcctcaagcttcttcccagtgaagatgtcgataccgacaaagttacatttagcatgtccgtgcttgccagtcttaga

SEQ ID NO: 240 - RNA, 328 nt, Spinacia oleracea
  source 1..328: mol_type = genomic RNA
ttagatccaccaccaatatccttgacgccacaaatttgttcttctcccatggcattctgaacactcacaatcaggtcttttccatccgcaaatccttccttgagctgcttaagaagagtttcatcagttggcagcttcaagtcatcctttgtacctccactctcagtcagcaggctaacaaaaccatcctcagctatgtcaatgagctgataatcggtacgattgacgtggggaacatcacaattgtgagaagatggaacaatatcttcaagcttcttcccattgaagatgtcaataccaacaaagttacacttagcatgaccgtgct

SEQ ID NO: 241 - RNA, 328 nt, Spinacia oleracea
  source 1..328: mol_type = genomic RNA
ttaagctgtttcagcagagcatcatcagttggcagcctcaagtcatccttagtgtctccattgtcagtcagcaggctaacaaagccatcctcagttatgtcaaggagctggtaatcagtacgagtgacatggggaacatcagaattgtgagaagatggaacaatatcctcgagcttcttcccattgaagatatcaataccaacaaagttacatttggcatggccgtgcttgccggtcttagaagtggaaacttcaacaaccttgcaggggcgattcttgatgacgatgtaaccgttcttacggatagtaccagcttgctgagggaaag

SEQ ID NO: 242 - RNA, 327 nt, Spinacia oleracea
  source 1..327: mol_type = genomic RNA
gagcatctcttaaagattctgcttttgcaactaaagcagtaactgagacacttccttctttcgtaaacttcacagcattaccaataacattcaatagaacctgcattagacgtttttcatcaccaacagcaaactctggcacatccgttgccaaattcgtcgttatcaacaactttttgatagatgctatgggcttgatcaaattaaggacctctctaaacatcgtgtggagattaaatactccattatcaagcttcaaacttccatcttcaagccgtgaaaggtccaaaacatcatttatcagagtcgcaagaagattactgcttt

SEQ ID NO: 243 - RNA, 328 nt, Spinacia oleracea
  source 1..328: mol_type = genomic RNA
tttacattatcatcatcagcaatcaacactttcaaccctctgaaccgagagtttgaagaaactgtgtgctctgtcatatataagtttccttcaggaatatatctttgttgttgaaatcgtagaacaagagtcatgttctgtggtatcccatttgagtttagagatatccatatatcaccctgcatcatctgagcaagccttttgcacatgctgaagctcaatacttccttgtttgaatcattctgggttctcctaatagtgtctgtcagataacctccacttatgtcaaactcaaactttactttaatgtactcctcgacagtagccg

SEQ ID NO: 244 - RNA, 328 nt, Spinacia oleracea
  source 1..328: mol_type = genomic RNA
gttgctgaaactctgaaaattccttccttttaaaccatgagtcaaaatgatctcagtcctattctcattgggcatccaaacagcacaagtaagcaaatccaaggttttagagagctcaaccatagttgtgtgcaagattgtatgcctatctaaggacttgcgaatctcatgagtaagcatcctaacatgccatccagcttcactttgtttcttaatcatcccaacctccctacccaaatcccaagtcttcttctttaacatgaattctctgaccttgactttgagaagcaaagggatgagggtgataagggtaatggcagtagcacaa

SEQ ID NO: 245 - RNA, 328 nt, Spinacia oleracea
  source 1..328: mol_type = genomic RNA
ttcacttctacttgaaactagttgactagcctcaaaccgaaaatcttttgatgcatttctcgctgaatcagttaatgcctgggtccctcgtctgctaggaggaacaaatcttgatggaagaaactctctgatggcattagatgctcttccaatattttgtggaggattactagattcagaacactggggttctctatcatgaccaacaaactgagaatgtacttggtcatttggtataggattgtttacgtcgggcagctttctaccaagttgcttaagatacataggatctcctgcatcttgctcattcacaactgcacctgtagaa

SEQ ID NO: 246 - RNA, 326 nt, Spinacia oleracea
  source 1..326: mol_type = genomic RNA
agagtgtacatattttaagccacgtaagagctgatataagaagtactggcaatgatcatcagtcaaagcttggttagagcgaattatctgatgaagatcagtatccatcagttcataaacaatgtaaacatcattgaatgcttctctctttggtggtcgaatgatatccttaatcgcaataatattttcatgatccatatggcgaagtagtttgatctctctcagtgtcctttttgcatctactatattgtcgaatgcattaccaatcttcttgatagcaatttcatggtgtgtttctgaattaacagcagcacaaacaaggccag

SEQ ID NO: 247 - RNA, 327 nt, Spinacia oleracea
  source 1..327: mol_type = genomic RNA
cgagtgtatatattttagcccgcgtaggatttgatataagaagtactggcaatgatcttctgtgagttcctggttagagcgagtaatttgatgaagatcagtatccattaattcataaacaatgtagacatcattgaacttctctctttcaggtggtcgtatgatgtcctttatcttgataacattgtcatggtccatgtgacacagaagtttgatttctcgcaaagtccttttagcatcaattctattgttaaaagcatctgcaatcttcttaatggcgacctcttcttgtgtctctgaatttcgtgcacagcaaacaatgccgta

SEQ ID NO: 248 - RNA, 327 nt, Spinacia oleracea
  source 1..327: mol_type = genomic RNA
agagtgaatgtacttcaaccctcgtaatatttgatacagaaaataccggcaatgatcatctgtcaactgctggttggatcttattatttggtgaagatcagtatccatgagttcatgaaccatgtatacatcattgaagttctctttctgaggaggtcgtattaggtccctgatggaaataacattgtcatgattcatgtggcgaagaagtttgatttcgcggagtattcttttggcatcaattctgttatgaaatgcattgctaaccttcttgattgcaacttcttctcgtgtctctgaattgacagcagcacagacaataccata

SEQ ID NO: 249 - RNA, 328 nt, Spinacia oleracea
  source 1..328: mol_type = genomic RNA
atcttataccttgtatcccaatgcaaatgaccgcctttcttaccgtgtatcacctcccctaagcttccattaggcatgtactcatacactaacagattagtctcatggttggagcaaaaccctaacaatctaacaatatgcctatgtctaatcttccctaaagtctgaatctcagcattaaacccatgatcatgagatgaacccctactcattacaggaagacgcttaacagcgacttgatcaccatttggcatatttcctttgtaaacaatcccagcaccacctttcccaataatgttatcttccttcaagcaatccaacacatcat

SEQ ID NO: 250 - RNA, 354 nt, Spinacia oleracea
  source 1..354: mol_type = genomic RNA
atactgtaattcttacacatggatgccaacatttttgtcagcttcttattctctatgttcatcctgttcaactcctcttctaaattcccactttttctctcgtccacctgtgttttgtataagggtggaatcagaagcttgagtacgaacagcaacccttgtgatcttagctttaatagtctcttctttaggttttccataggaatcctcttcatcactggaactactctcaccatgtaacctgcatactctcagcttcagcaactcttgctctatcctgaattgcatcattcaaggacattttctgaaccattggcctaaccaagttaaatgtgggggaatttggaattggag

SEQ ID NO: 251 - RNA, 324 nt, Spinacia oleracea
  source 1..324: mol_type = genomic RNA
acttaaaacatgaataatccaccaccttcccttcgttttttgattcattcttttccattgcaagcttcattatgctttcggccttgtcgttgggtagatcataaacagtaattgttgagattgtaacgttggtgttattgttgtagatgtacatatttcttgtatcgtttttctacttgctaactgtattatgctttttgcctgaacatcggtaacataggcagatcagaaacattaggcacggtagctggtatgacgttcttcacaaattttggagcaacagtcaagttctggtttgatttctgggttggaatcagggtctga

SEQ ID NO: 252 - RNA, 311 nt, Spinacia oleracea
  source 1..311: mol_type = genomic RNA
tatctcattttccagtcgtcaatcatttcattaacaacatcaatagcttctccgagattcgatgcttggaaaccggtggagacaaacgatttaaaaagctgggtataactctcagtcttaatgcatttcccttctagggtttctgactccttgaacagaatactggaatattattcttgtaggcccaaaaaaggtatgaactcttgtcatttatttcttttcccaaacgagcgatcaactttgagggtgtccatgttatcttctctgtattttgctcttccagcattttatcaaaaattggaacaatccaa

SEQ ID NO: 253 - RNA, 354 nt, Spinacia oleracea
  source 1..354: mol_type = genomic RNA
taatcagtacgagtgacatggggaacatcagaattgtgagaagatggaacaatatcctcgagcttcttcccattgaagatatcaataccaacaaagttacatttggcatggccgtgcttatccttgacgccacaaatttgttcttctcccatggcattctgaacactcacaatcaggtcttttccatccgcaaatccttccttgagctgcttaagaagagtttcatcagttggcagtctttcccctcagcaaatccatccttgatctgtttattcagaacttcatcagttggcagcttcaagtcatccttagtatctccattatcagtcagcaggctaacaaaaccatcctcag

SEQ ID NO: 254 - RNA, 324 nt, Spinacia oleracea
  source 1..324: mol_type = genomic RNA
tttgatttcgcggagtattcttttggcatcaattctgttatgaaatgcattgctaaccttcttgattgcaacttcttctcgtgtctctgaattgacagcagcacagacattaattcataaacaatgtagacatcattgaacttctctctttcaggtggtcgtatgatgtcctttatcttgataacattgtcatggtccatgtgacacagaagtttgcttaatcgcaataatattttcatgatccatatggcgaagtagtttgatctctctcagtgtcctttttgcatctactatattgtcgaatgcattaccaatcttcttgat

SEQ ID NO: 255 - RNA, 324 nt, Spinacia oleracea
  source 1..324: mol_type = genomic RNA
atcattgatcaaaagagaaaggacattccctgtcttcatcattgtgtcaacaataagtttctgttcactgcttaaattctcatcttttagaactgaaagcaaccccaaatacttccttgtttgaatcattctgggttctcctaatagtgtctgtcagataacctccacttatgtcaaactcaaactttactttaatgtactcctcgacagtagccgtaaagcagtaactgagacacttccttctttcgtaaacttcacagcattaccaataacattcaatagaacctgcattagacgtttttcatcaccaacagcaaactctgg

SEQ ID NO: 256 - RNA, 300 nt, Spinacia oleracea
  source 1..300: mol_type = genomic RNA
ggcatgcagtagatcgacatccacccacgagcatgcatcttaaatccagtcaaaatatcttctgtcactgaaccatatatccacccaatctctttaccccattcagacttgtcctcgtatccacagctaataacatgaatggcttctttctccgagatccaccaaagcatgatgataggatacctttcttcttgggctttggtttgataggaggctcataaccatacaaagcagttctgttgaagacacatcccgtacccacatagacaggtccttggataccatctaaacctctcaaat

SEQ ID NO: 257 - RNA, 308 nt, Spinacia oleracea
  source 1..308: mol_type = genomic RNA
agtctgctgactgaatatcttctacctcttcaaccatttttatacgacgctgaggcccagtagcgccatttattgatcttctgcaatcctctataacatcactacttcttgcggaacaatctcctttgttaaaggagatggattgggttgcatcagaaatacaatcgccaacaatgttattcctaacgtgatcatctttattgtctatagatgagttatttatcaaagtcagagtgctgagggtacttccaaaagatttctcgaagtttcttgcaaaagcatcaaacttgtcgccaaatacattggtt

SEQ ID NO: 258 - RNA, 345 nt, Spinacia oleracea
  source 1..345: mol_type = genomic RNA
gtggtgcccatcctggtcagtctgctgactgaatatcttctacctcttcaaccatttttatacgacgctgaggcccagtagcgccatttattgatcttctgcaatcctctataacatcactacttcttgcggaacaatctcctttgttaaaggagatggattgggttgcatcagaaatacaatcgccaacaatgttattcctaacgtgatcatctttattgtctatagatgagttatttatcaaagtcagagtgctgagggtacttccaaaagatttctcgaagtttcttgcaaaagcatcaaacttgtcgccaaatacattggttgccacaacatcgaggacgg

SEQ ID NO: 259 - RNA, 30 nt, synthetic construct
  source 1..30: mol_type = other RNA
gactaacatcattcctattgacagcgccgc

SEQ ID NO: 260 - RNA, 30 nt, synthetic construct
  source 1..30: mol_type = other RNA
gacyaacatcattcccattgacagcgccgc

SEQ ID NO: 261 - RNA, 28 nt, synthetic construct
  source 1..28: mol_type = other RNA
catttgtatccggaatccaagacgccgc

SEQ ID NO: 262 - RNA, 28 nt, synthetic construct
  source 1..28: mol_type = other RNA
catttgtatccggaatccaagacgccgc

SEQ ID NO: 263 - RNA, 279 nt, synthetic construct
  source 1..279: mol_type = other RNA
ggaccgtgtatggccaaacaatgtagaatgtatgaattaaggaacagggagcgtatttctgtagcagctgcttccaaattgatggcaaacatggtatacaatcacaagggaatgggactgtcaatgggaatgatgttagcaggatgggataaaagagtaaactccgagggtaccagaacacctggaaaagtctttagtgtgggttcaggttcaatttatgcatttggtgtcttggattccggatacaaatgggatttgacggatgaggaggctacgatt

SEQ ID NO: 264 - RNA, 281 nt, synthetic construct
  source 1..281: mol_type = other RNA
ggaccgtgtactggccaaacaatgtagaatgtatgaattaaggaacagggagcgtatttctgtagcagctgcttccaaattgatggcaaacatggtatacaattacaagggaatgggactgtcaatgggaatgatgttagcaggatgggataaaagagtaaactccgagggtaccagaacacctggaaaagtctttagtgtgggttcaggttcaatttatgcatttggtgtcttggattccggatacaaatgggatttgacggatgaggaggcttacgatt

SEQ ID NO: 265 - RNA, 1840 nt, synthetic construct
  source 1..1840: mol_type = other RNA
atgattaacatcaattcgcgagagttgaactatctagtgtaccgttatctacatgaatcaggtttcacacatactgccttcacattaggttatgaggctgggctgaataaaagcacaatagatggaaatttagttccacctggtgctcttgttacatttgtgcagaaaggaattcagtatcttgagctagaagctaacctcacatgcaatgattctgatatcgatgaagatttctctttcatacaaccgatcgatctcatcacaaaggatgtttatgaactacaaaaaatgataaaggagaagaaagaaagtcttcagaaaactaaaaacagagaaaaaaacaaagaacatgagcaggaacatgcaagagaaaaggagaaagaacatgtgaaggaaagagagaaagaacatgaacaggaaaatgctagagaaagagaaagggaacgagggcatgctagagaaaaagaaaaggaacaggaaagacaacacagagaaagagaaaagagaattgagcaagaacaacatagagaaaaagagaaggaaaaacaacaaagggaaaaagaacgtgagttggagaaagagaagatagagagggaaagagaaagagaaagagataataagggtaaacacaaagagaaagagaagaaaaatgaggatcttgaggataagatacgagtccaacctgaagaaaatgtgattgcagaaggagctgaaccaatggagattgacacaagctcaacagctttgccttctgttatccccaactctgatgtgacaattttagaaggtcatacttctgaggtttttgtatgcgcatggagtccaactggctctcttttggcatctgggtctggagattccacagctcgaatttggaccattggtgatggaccctgtagttcaaatttacaaaaaggccccttagatgttgctgttttgaaacactacagaggtagaacaaatgataagagcaaagatgtaaccacacttgactggaatggtgatggaactttacttgcaacaggttcttatgatggacaagccagaatatggagtaaagatggggagctgatgagtacattaacaaaacataaggggccaatattctctttaaaatggaacaagaaaggagactatcttttaagtggcagtgtagacaaaacagcaattgtatgggacattaagactggagaatggaaacaacagtttgagtttcatgcagctcctactcttgatgttgattggcgcaacaatgtctcatttgcaacttgctcaactgacaacatgatttatgtctgtaaagttggagataatcgaccagttaaaacttttgtgggccatcagggtgaagtgaatgccatcaaatgggatcctactggtacacttctggcttcatgctcagatgattctaccgcaaagatatggagcgtaaagcaggacacctgtttgcatgatttaaaggaacatacaaaggaaatatacaccataagatggagcccaacaggcccaggaacaaataaccctaaccagccgctagtgctagcaagtgcatcgtttgattcaacaataaagctatgggacgtggaaacagggagcctcctccacagcttagttgcacatggggatccggtatattcggttgcattcagtccaaatggggagtatttggcgagtgggtcattggataaatgcatgcacatatggtctgtgaaggaagccaaagtggtgaaaacatatgttgggagtggtgggatatttgaagtttgctggaacaaagaaggtgataagattggtgcatgtttttcaaacaatgtagtttgcattttggactttcgtatgt

SEQ ID NO: 266 - RNA, 2697 nt, synthetic construct
  source 1..2697: mol_type = other RNA
atggagaagaagaaaggcatgaagaacatcttcaagaagcttcacataggaagcaatcacgatgctaatcgatcatccaatgatccaacgtcgtcatcgtcttcgtcgtcttgcgccaccgccgaccaccgtacatcttcaactctcaccggacaaatctccgtccaatctccagctgctgccgcaagtcctcctcaaacgtctccgaccgtcaatgtaacatcgcgtcaacaagattattattcgtcagaagaagagtaccaggtccagctagccctagctttaagcgtgtcgaattcggagtctagaggtgataccgatagtgatcagatccgtgctgcgaagttgttgagtttagggcaacattcgagtaataatcatgtacctgatagaggtgatgcggcagctgataagttgtctcggcagtactgggattatggtctccttgattatgaggagaaagtagtggatggtttctatgatgtatatgggctttcaaccgaaacaacatcccaagggaaaatgccctctttgtcagatcttgaaacagatcctcaaacttcaggttttgaagtaatcatagtaaacagaaccattgatcctgccctagaagagctgtttcaggtagcccactgtattgcattagactgccctcctgctgaggtgagtcttttagtgcaaaggcttgctgaacttgtgacagaacacatgggtggaccagtgagggatgctaatatcatcatggcaagatggatggaaagaagcacagaattgaggacatctttacataccagtatataccctattggatctttaagagttggactgttacgccatcgtgctttgcttttcaaggtactggcagaaagtgttggaataacctgtaggcttgtcaggggtagtcactatactggtgttgaggatgatgcagtcaacattatcaagttggataatgaaagtgagtttctagtggatctaatggcagcccctggaacacttatacccgcggatattttcagtggaaaagattcctccttgaaaccaaacatcccaaaatcaagtcaacttccaggtgtccagtcaactaaagatttctcacttccttcaagacccgtttcaaacaataatatcaatggtgccagtagtcaaaacattgaagtgcaaaacaacactagtcaattagatcacatcccttcatcagcaatcggaaattctctttacaaaggaggccgtgggcccaatgcagttagtggtggctcaaggatgaatgttaacatagtcccgtataatacccaaaatacccctggccctgaggacaataaaaaccttttcacagagctcaatcctttccaaatcaaaggatccggtaaggcttcaatgcaacaggctgataacaaaaacacaaaaatcaaccaagttgtttccggaagacctccggtcccacaaatgtggaaaaatcgtcatgctgttaatgaggtagtagcgggacccggaccaggaagggtatattctgatgggctgaagtcacgtgatagttatggtgggactttaaatcaaaatagtgaaacgggttcttcgaataatgctgctgagtcggaagctgcatgggctgagtggaaaaactatagattgtctttggaggttgtggggaataataataataataataacaacaacaatgctttctcaaggaataaggaaaagttgaatgtggtgagaggggaagaagatgttagaaaaggttcagagtatcgggtcaatgttgaaaaccgaaaggtggtgggacccgaaagctccacgtcatcatcatcatcgtcatcggttgttgatcctgtgattgatgatgttggagactgtgagattccgtgggaagatttggtcattggtgaaagaattggactaggctcatatggtgaggtttatcatgctgattggaatggcactgaggtggctgtgaagaagttccttgatcaggatttttcaggtgctgctttagcagagttcaagagggaagtgcgcattatgcaaagattacgccatccaaatgttgtgctttttatgggtgctgttactcgtcctccaaacctttcaatcatcaccgaatttctccccagaggaagcttatacaggataattcatcgcccacattgccaaattgatgaaaaacgaagaatcaaaatggctcttgatgttgcaaaggggatgaattgcttgcatacaagcatcccaacaatagttcacagggacttgaaatcaccaaatttgttggttgatgaagattggaatgtcaaggtatgtgactttgggctgtctcgcttgaagcataacacatttttatcatccaaatcaacagcaggaacgcctgagtggatggcacctgaagtcctgcgtaatgaaaattcaaacgagaagtgtgatgtgtatagctttggtgttatactgtgggagcttgcaaccctaagattgccctggagtgggatgaacccgatgcaggtggtgggtgctgtgggcttccaaaaccgccgcctcgacatccccaaagagttagaccccttggtcggaagaatcatctgggactgctggcaaacggatccaaacctacggccttcgtttgcacaacttacggtggctctaaagccattgcagcggttggcagccactacacaaatcgatgatgatcaaccaatcgatatttaa

SEQ ID NO: 267 - RNA, 375 nt, synthetic construct
  source 1..375: mol_type = other RNA
atggcttccaagattcttgcaaatttgattgtaatgggttctggaataatggctagggctcttgttcaagcatatagacaagccctccaaaatgcatcaaaatcaggtgttgctcaagacacattacaaaatgttgtgcgtaaaggaagcaaagctatgactgaacaagaagccagacaaattcttggtgtcaatgaacaatcatcatgggaagaaatcgcacaaaaatatgacaatttatttgaaaggaacgccaagaatgggagtttttatctacagtcaaaagttcatagggctaaagaatgtttagaaacaacttatcaagcaggtgaatatgcaggtactcctgaggatcgcgagatggcgaagacttga

SEQ ID NO: 268 - RNA, 357 nt, synthetic construct
  source 1..357: mol_type = other RNA
atggctgctaagattcttgcaaatttgattgtaatgggctctggaatcttgattaggggaatggttcaagcatatcgtcaggctctccaaaatgcctcaaagtcaggggttgctcaagaaacattacagaatgctgtgcgaagaggaagcaaagctatgactgaacaagaagcgagacaaattcttggtgtgactgaacaatcatcttgggaggagattgcacagaaatatgataatctatttgagagaaatgcgaagaatgggagtttttatttgcagtcaaaggttcacagggccaaggagtgtttggagacggtttatcaaccgaaagatcatccaggtgatcccgttggttga

SEQ ID NO: 269 - RNA, 3440 nt, synthetic construct
  source 1..3440: mol_type = other RNA
atggccggcaagaccgacttcgattcttctctccctaactctcagatctccagagatgtccaagggtctgacagttctattccactttcaccacagtggctcctgtccaaacctggggagaacaagatgggaactccaacctcggaggtccccaccagtccttttccaccatttgccaaccgtttggatttcatgaaatcacctggaaccgatgattcccagaagaaaaaagatgtgtttaggccttctgttcttgaaagccgtgaacgttggcgtgatgaggaaagagacacaaattcttttgtccgcaaggataacaggtggcgggagggggaaaaagagcttggggacacccgcaaggtggcagacaggtggacaacagataacaacataacaccaggaaaacaacactatggcgaatccagacgtgcaccttctgagaaatggggtgatactggaaacaaagatgcacgtgagagcaaatggaacacccgatggggccctgatgacaaggatgctgatgtcacacgtgacaagtggacagaatctggaaaagatcatgatgtccaggtgtcccatgagggtggtgaccaccctcgaccatggaggcctagaggaagagcagagccgccacatcagcaaactccaggggtaaacaagcagacttctatgtttgttcatggtcggggtcgtggggagaatcctaattccacattttctattggccgaggaaggggtgggacccaccctatgaataatagttccagttccactcatttgcagcctttaggatcttttcctgacatgggtggtgataatacatctcctatgaggtatagcaggtcaaagctgcttgatgtgtacaagataactaatatgaaaggtggtgaaaggatgttggatgtgccttcacttacacaggaggaacctttggagcctcttgcacttgtttcacctacccctgaagaattgtttataatgaaggggattgacaaaggggacattttaagtagtggtgctcctcaaattactaaagatggatcatctggggttaacatggttgatacacagtcaagaagaaccaaacttgggagtagagaagatctaccacttgcttctgacaattattcggattatgtggagggcttatctcatgagaaacaagggcactcatatgagaccaatttcaaacctaatctcatggaagatcgccagtcttttatagattacaaatccaaaaatgaagcaataggaacctcagctaactctggggcaaactggagatctttatccatggggcaacgtgcacaaccgaatacttccaatgaatggagtagcagtttaacaaatgtcacaaatgtttcaaatgtttcaaatgttacaaatccaggtatcaaaaggcaacaatctggaaacactatggatagggagcatgaagcaaggatactttcacaatcacagccatcacctgaagatttggttcttttttataaagatccacaaggttcaattcaaggtccttttacgggaattgatattattggatggtttgaggctggatattttgggattgatttgcaagtacgtcttgcaaatgcaccaaatgactcaccttttgctttacttggtgatgtcatgcctcaccttcgtgcaaaagccagacctccacctggatttactgctgctaaaagtgaaatcaatgatgaatccagtattccaaatcctaatgctatgatgaaaaatgatccaaggtttcaacatgggtcaaccacagaagctgagaatagattcattgagtctctcatgtctggtagtggtggctcattggagaaatttgggctttctgaaggtatgcaggggtattttgggaaccctggttcgattccagctatggaaattgcaaaattgatgcaacttgaaaggcaaaaatctatgtctagtccttactctctttggtctgggccccctaaatctgacattcttcaagatccttcagttttacctcaaaatcctccaaatccagagttcatgtctattcttcaaggcttatctgaaaggtcaaactctgctgcaaacagtggggtgagtaactggtcaaacttccctgttcaaaccgggttggacccacttcagggtaaacatttccctcttcagacaccattcggggcccaccagaggctgcaaggacagaatctaccctcactgggtccactaacacctgaaaagcttctagcttctggccttcctcatgatcaaatcttaagcttattacaacaacaacacatgtcacagataaaacctcaaatccctacaccacagttgtctgtattggatgagtacctgttacttaaacaacaacaacagaaacaggaacagcttcagcaacaacaacagcagattatgagacaacaactgctgtcccaagtcctcatagagcaacaatctctgcaacgttttggtgagcaacaacagcagtctgttggatttgctcctccacatgaggtgttacagatgggatcacagcatcaagggcttaatactaacactcctcatatggctcaatttgttcccaccccaggtcttgaagattctattcatttgccacatcagtttattgacaacagagtggaacaaaaggttgttggagatgagattgttcaccagaaggaacctgatgtacaggaacaggatcagccattgatgattgattcaagaacagatgaaaatgttgctttgatttctgaccctctggttgctgtgaaaaatgaagttttagagactgaatttgttgataatgtggaggtgatgccagctgtcacggaggcaccaaaggtccagaaaccatctactaatgagtccagtgttgtgaaagaagtaaagcctgttgaaactgtgaaaaaagccactgaaaagaagtcaaagaaacagaagtcttcaaagtcacaatcgtctgacatggctaaaacagtatctaaaccccaacagcagcagccaaaggaacctgaaactgatttgaccaaagtcaacgaggtcgaaatggaaacatctcaagtagaagagaccaaatctagagtagccatggaagagcctgtatctatgtctagtaatgttaaccaaatccctcaagataacaacacacaacaagttcatattcaacgtgcatggaagcctgctcctggttttaaaccaaagtcactgttagaaattcaacaagaagagcaacgccgggcccaggcccaggcccaagctcaggcccaagcccaagcagagatggctgtttctgatatgtcaacatctttagggtcaatgaacatatccagcccttggtctggttttgttgccaattcagaccacaaacttacagaaaacaagaaagattgggccacttctgaggcttcccaaaatcaaaacaacaagagccagttgcatgaacttttaac

SEQ ID NO: 270 - RNA, 1149 nt, synthetic construct
  source 1..1149: mol_type = other RNA
atgtccgaggcggctgcggcccctcctccgtcgcagcagcccttgaccggaatcgacaacatcccggcgaccctcagccatggcggtcgtttcatccagtataacatctttggcaacatcttcgaagttactgccaagtataaacccccaatcatgcccatcggcaaaggcgcttacggcatagtctgttcggctttaaattcggagacgaatgagcatgttgcgattaagaagattgccaacgccttcgataataagatcgatgcgaagaggacactgcgagaaatcaagcttctccgtcatatggatcatgaaaacgttgttgcaattagagatataatcccacctccagaaagaacatccttcaatgatgtctacattgcttatgagctcatggacactgatctccaccaaataatccgttctaaccaatctttatcagaggaacattgccagtattttctgtatcaaatccttcgtgggttaaagtacatacactctgcaaatgtgttacacagggatttaaagccaagcaatcttttattgaatgcaaattgtgatttaaaaatatgtgattttggattagcacgtgtcacatctgaaaccgatttcatgacagaatatgttgtgacaagatggtatagagctcctgagcttttgttgaattcatctgattatactgctgccattgatgtgtggtctgttggctgcatattcatggaactcatggataggaagccattgtttcctggtagagatcatgttcatcagcttcggttgctaatggagcttattggaacaccatcagaagctgaattagggtttctgaatgaaaatgcaaaaagatacattcgacaattgtctcattatcaaagacaatcattcaatgaaaagtttcctcaagttcatcctgcagccattgaccttattgaaaagatgttgacttttgatcctagacaaagaattacagttgaagatgcattggcgcatccttatctgacatcactgcatgacatcagcgatgagccagtgtgcttgacgcctttcaactttgactttgagcatcatgcacccaccgaggaacaaatgaaggagatgatttacagggaagctcttgcctttaatccagaataccaacagatgctgtga

SEQ ID NO: 271 - RNA, 1614 nt, synthetic construct
  source 1..1614: mol_type = other RNA
atgtcttccttttccgaccaagctcctgctccccggaccggcggactcgcagctcgaatcgcagaaagagtgggctccggcgtaccaaaattcaagtcaatccctccaccttcactacccatctcccctccggcggtatcaccttcttcctacttcgctattcccgctggtttaagcccggctgatcttttggactcccctgttcttctctcgtcttcccacattctaccatctccgaccaccggttccttcccatttcaagctttcaattggaagaatctcagcagcaacaaccagaaccacgaacagagcataaaaaaagagcaaaaaagcttcacagatttctcttttcaacaacaatcaaatcatctaactgagcaactatggaatcatcagaaatccagggaacaagatgaaaaagcttttgtgcaatcagaacacgcatctcagttccagagtttctcccctgaaatttccacgattcaaaccgattcaaattcgcagacgcaaagttttcaatctggttatgcagccagcaacaacaatttgaacaatcaaccatcgcagaaaaaatcagatgatgggtataattggagaaaatacgggcaaaagcaggtgaaaggaagtgaaaatcctagaagctattacaagtgcacgtatccaaactgttcgatgaagaagaaactggaaacgaatttagatggacagattacagagatagtttataagggtaaccataatcatccaaaacctcaatctactcgaagatcatcgtcatcgtcatcagcttcaaatcatttacagatgactcaagtgtctagtaaccatgaactacttgattaccctgatcaatcttatggttctcacggatctgggcagttcgattccgtagccacgccggagaattcttcaatttctgatgaccttgatcggagtagctgtcagaggagtaggtcaggtggggacgagttcgatgaagacgagcctgatgccaaaagatggagggcgtcggaaaacgaagggatatcaacgatcggaggaactagaactgtaagagaaccgagggttgtggttcaaacaacaagcgatattgatattcttgatgatgggtatagatggagaaaatacggccaaaaagtagtcaaaggaaaccctaatcctaggagttactataaatgtacgagttccggttgttctgttagaaaacacgttgaaagagcatcccaagacttgaggtcagtgataacaacctacgagggaaagcacaaccacgatgtcccaatggctcgagggtctggtcatcggttgccatctggcacactcggcaacaacattcccactactgcaattaagcctgttgcactctcccattaccaatccaataactctatttcggattcagcccaccgtggattcagtttcctttcatcagcagataatcaaaccatgtttacatcagagatgttgcagaataatgaaagtttcaagttctcgagatttgagaatgctttgaagtccaactacattgatcaaaaacgtgattccgaaagagcattttcagaagccaaagaggaacctcgagatgatatgtttttcgagtccttattatgctaa

SEQ ID NO: 272 - RNA, 1485 nt, synthetic construct
  source 1..1485: mol_type = other RNA
atggcttcctctggtggcagcttgaatacttccactacgacttctcagtacccacctttctctttctcgacgtctttcagtgatcttattgcttctaataccggcgaagatcccaattgggggttcttcagtggcggcggcggcggcggcggcggtggtggcgaaagtccaaaaccaactccgacgccgttctctccgtctgctttcctttctacaccaactgggttgaaccaaaaccagttcttggattctcctttcttccctttcaattcgaatattgttccatctccgacaaccggaagcttttctggtttggatttcaaggatgaagaaagaacttattctaacttttcatttcaatcacaagcaaggcctcttgcttccatggcttcaatcaagatggaagaacaagtaaaaactcaaccacaacaatggggaaaacaaactgatttcacatcagaaatcaacaaaaataagtccgaatcaacacaatcggtccaaacccaatacgtttctgatccatctcaagccgttaaagatcaaacaagattagaagatggttacaattggagaaaatacggtcaaaaacaagtcaaaggaagcgaaaaccctagaagttattacaaatgtacgttccctaattgcccaacaaagaagaaagttgaaagaaacttagaaggacatatcacggaaatcgtttacaaaggaaatcatagtcatgcaaagcctcaaaatgccaagaaatcatcttcaaactcttatagtgaagcccccattgaaagcaatcatttcgattcttccgcatcatttggggaagatgagtttgagcaaggctcttcaattagtaaatccggtgatgaccatgaaaacgagcccgaggcaaaaagatggaagggtgagaccgagagcgaagctatttcgggtcccgggagtcgaacggttcgggaaccgaggattgtggttcaaaccacgagtgatattgatattcttgatgatggttatagatggaggaaatatggacaaaaagtagttaaaggcaatccaaacccaaggagttactataagtgcactttcaacgggtgtccagtgagaaaacacgtagaacgcgcatcacacgacttacgtgccgtgatcacaacatatgaagggaaacacaaccacgatgtgcccgcaccacgtggtagtgggagctacgccattaatcgaccaccaacaactaacaacggtaacaccaataacaactacggactcatggccataaggccatctgcaaactactcaagtagtttgcagagtacaacggggttgnnnnnnnnnnnnnacggtggtcaacccccgtacacactgcaaatgttgcagaactccgggagcaatgggctcccggagagttcttatgtgaatcagaaccggggaaaaaacgggtcgttatcgagcgctaaggaagaacccgaagatgagttgttttacaactcttttctagggtga

SEQ ID NO: 273 - RNA, 879 nt, synthetic construct
  source 1..879: mol_type = other RNA
atgtgggaggggggatcgtcgtcgatgacggtggcggaaggaggtggaggtggtggaggagggaggaggaaaccatcgtggagagagagggaaaataaccggcggagggagaggcggaggagagcaattgcggcgaatatctataatggactcagagcacagggcaactacgatttgccgaaacattgcgataacaacgaggttttgaaagctctttgtaaagaagctggttgggttgttcttcctgatggcaccacctttcgcaagggatgcaagccaccacccgccattgaaatcggaagcacatcaaccaacacaactccatgttcttcccgaaaaccaagtccaccttcttcatcattcccttctccatcacacttcgacatgaatcaatccgcatcacatcaccccttcaccttcttccgtgactcaattccttcatcactcccacctcttcgcatatcaaacagcgcccctgtaactccacctctctcctccccaacttcaaaacctccccaaaacaaccatctaatctgggaatcattcaccaaacagtcaattccctctttcaatctcccctactttgcatcttctgctcccacaagcccaactagatatcaacgtttcacaccctttactatcccagaatgtgatgaatccggttgctccacccttgattcttgtcaatgggtgagtcgacccagttctcccaccttccaccttgtgagaccaatggctccggaaatctccaccatggaaaaagggaaaggaatggaattggaatttgggaatggagtgaatgcatgggaaggggaaaggattcatgatgtgggtttggatgatcttgaactcacccttggaagtggcaatgctaagtga

SEQ ID NO: 274 - RNA, 1806 nt, synthetic construct
  source 1..1806: mol_type = other RNA
atgatgcaggaagggggatcatcgtcgttgacggcagcgggggaaggaggagggaggaggaagccgtcgtggagagagaaagagaataatcggaggagggagcgacggcggagagcgattgcggcgaatatatataatgggttgagagcacaggggaactacaatttgcctaaacattgtgataacaatgaggttttgaaagcactttgtcaagaagctggttgggttgttcttcctgatggcaccacctttcgtaagggttcgaagccaccactaccttccattgaaaccggcggcacatccaccactacgacgccgtgttcttcccagaaaccaagtccgccgtcttcatcgtttccgagtccgatcccttcatatcaatgcagcccatcatcttcatcgttcccaagtccatcaagtaacccatttgcatttcttcgaaacaccattccatcttctctcccacctctccggatctccaacagtgcccctgtaactccacctctctcatctccaacttcaaagtttcctaaacccaacaatctcaactgggaatcgttcacaaaacaatcgatttcctcattcaatttgccattttttgcgtcgtctgcgcctacaagccctaccagatcccaccgcttcacacctgcaactatcccggagtgtgatgaatctgattgctccaccattgattcatgccaatggttgcgattcaagaactatgaaccgatggtgatgacaaaccccaactccccgacttataacttggtgaaaccggtggctagagccactgccgccatggatgcgagctcggagaaaggtaaagggatggaattcgagtttgagaatggaggagtgaaggcatgggaaggcgagaggattcatgatgttggagtggatgatcttgaactcactcttggaagtgggaattctaaaatataaatgatgtgggaaggaggatcgtcatctgcggttacggaggaaggtagcagcggcggcggcgggggtggaggagggaggaggaagccgtcatggagagagaaagagaataaccggaggagagagagacggcggagggcaattgcggcaaacatatacaatgggttgagggcacaaggaaattatagtttgcctaaacattgtgataacaacgaggtcttaaaagctctttgtaaagaagctggttgggttgttcttcctgatggcaccacttttcgtaagggatgcaagccgccaccctccgccttcgaacacggtggctcatcaacaaacacgactccgtgttcttctcggaaaccaagtccaccgtcatcgtcgttcccttcataccaatgcagcctctcatcttcttccttccccagcccatcgcatctcgattctacagatccgccattaaataaaccttttgcttttctaagcaactcaattccgtcatcactcccacctctccggatctcaaacagcgctccggtgaccccaccactctcatccccaaaagttccgaagaaaaacaatctcaattgggaagcttttgatctaccatgtttcgcttcttcatgtgaggagcctgattggtccaccggcggttcgtttcagaggttgaggttccaggattgtattccgatgatggtgaacccaaactctccaacgtccaatcttgtgaaatcaatggctcaagtggctgctatgaatgatgcgatcgctgaaaagggtaaaggggtgaaggcatggaaaggcgagaggtttcatgaagtcggttttgatgatcttgagctcacccttggaagtgggaatgctcaaatatag

SEQ ID NO: 275 - RNA, 318 nt, synthetic construct
  source 1..318: mol_type = other RNA
ttacaaaaaggccccttagatgttgctgttttgaaacactacagaggtagaacaaatgataagagcaaagatgtaaccacacttgactggaatggtgatggaactttacttgcaacaggttcttatgatggacaagccagaatatggagtaaagatggggagctgatgagtacattaacaaaacataaggggccaatattctctttaaaatggaacaagaaaggagactatcttttaagtggcagtgtagacaaaacagcaattgtatgggacattaagactggagaatggaaacaacagtttgagtttcatgcagct

SEQ ID NO: 276 - RNA, 318 nt, synthetic construct
  source 1..318: mol_type = other RNA
actggtgttgaggatgatgcagtcaacattatcaagttggataatgaaagtgagtttctagtggatctaatggcagcccctggaacacttatacccgcggatattttcagtggaaaagattcctccttgaaaccaaacatcccaaaatcaagtcaacttccaggtgtccagtcaactaaagatttctcacttccttcaagacccgtttcaaacaataatatcaatggtgccagtagtcaaaacattgaagtgcaaaacaacactagtcaattagatcacatcccttcatcagcaatcggaaattctctttacaaagga

SEQ ID NO: 277 - RNA, 307 nt, synthetic construct
  source 1..307: mol_type = other RNA
tctggaataatggctagggctcttgttcaagcatatagacaagccctccaaaatgcatcaaaatcaggtgttgctcaagacacattacaaaatgttgtgcgtaaaggaagcaaagctatgactgaacaagaagccagacaaattcttggtgtcaatgaacaatcatcatgggaagaaatcgcacaaaaatatgacaatttatttgaaaggaacgccaagaatgggagtttttatctacagtcaaaagttcatagggctaaagaatgtttagaaacaacttatcaagcaggtgaatatgcaggtactc

SEQ ID NO: 278 - RNA, 284 nt, synthetic construct
  source 1..284: mol_type = other RNA
atggctgctaagattcttgcaaatttgattgtaatgggctctggaatcttgattaggggaatggttcaagcatatcgtcaggctctccaaaatgcctcaaagtcaggggttgctcaagaaacattacagaatgctgtgcgaagaggaagcaaagctatgactgaacaagaagcgagacaaattcttggtgtgactgaacaatcatcttgggaggagattgcacagaaatatgataatctatttgagagaaatgcgaagaatgggagtttttatttgcagtcaaa

SEQ ID NO: 279 - RNA, 318 nt, synthetic construct
  source 1..318: mol_type = other RNA
aaggtccagaaaccatctactaatgagtccagtgttgtgaaagaagtaaagcctgttgaaactgtgaaaaaagccactgaaaagaagtcaaagaaacagaagtcttcaaagtcacaatcgtctgacatggctaaaacagtatctaaaccccaacagcagcagccaaaggaacctgaaactgatttgaccaaagtcaacgaggtcgaaatggaaacatctcaagtagaagagaccaaatctagagtagccatggaagagcctgtatctatgtctagtaatgttaaccaaatccctcaagataacaacacacaacaagtt

SEQ ID NO: 280 - RNA, 316 nt, synthetic construct
  source 1..316: mol_type = other RNA
gatgcgaagaggacactgcgagaaatcaagcttctccgtcatatggatcatgaaaacgttgttgcaattagagatataatcccacctccagaaagaacatccttcaatgatgtctacattgcttatgagctcatggacactgatctccaccaaataatccgttctaaccaatctttatcagaggaacattgccagtattttctgtatcaaatccttcgtgggttaaagtacatacactctgcaaatgtgttacacagggatttaaagccaagcaatcttttattgaatgcaaattgtgatttaaaaatatgtgatt

SEQ ID NO: 281 - RNA, 300 nt, synthetic construct
  source 1..300: mol_type = other RNA
tacaattggagaaaatacggtcaaaaacaagtcaaaggaagcgaaaaccctagaagttattacaaatgtacgttccctaattgcccaacaaagaagaaagttgaaagaaacttagaaggacatatcacggaaatcgtttacaaaggaaatttatgcagccagcaacaacaatttgaacaatcaaccatcgcagaaaaaatcagatgatgggtataattggagaaaatacgggcaaaagcaggtgaaaggaagtgaaaatcctagaagctattacaagtgcacgtatccaaactgttcgat

SEQ ID NO: 282 - RNA, 300 nt, synthetic construct
  source 1..300: mol_type = other RNA
atctaatctgggaatcattcaccaaacagtcaattccctctttcaatctcccctactttgcatcttctgctcccacaagcccaactagatatcaacgtttaaaagggtaaaggggtgaaggcatggaaaggcgagaggtttcatgaagtcggttttgatgatcttgagctcacccttggaagtgggaatgctcaaatatattgagaatggaggagtgaaggcatgggaaggcgagaggattcatgatgttggagtggatgatcttgaactcactcttggaagtgggaattctaaaatata

SEQ ID NO: 283 - RNA, 318 nt, synthetic construct
  source 1..318: mol_type = other RNA
agctgcatgaaactcaaactgttgtttccattctccagtcttaatgtcccatacaattgctgttttgtctacactgccacttaaaagatagtctcctttcttgttccattttaaagagaatattggccccttatgttttgttaatgtactcatcagctccccatctttactccatattctggcttgtccatcataagaacctgttgcaagtaaagttccatcaccattccagtcaagtgtggttacatctttgctcttatcatttgttctacctctgtagtgtttcaaaacagcaacatctaaggggcctttttgtaa

SEQ ID NO: 284 - RNA, 318 nt, synthetic construct
  source 1..318: mol_type = other RNA
tcctttgtaaagagaatttccgattgctgatgaagggatgtgatctaattgactagtgttgttttgcacttcaatgttttgactactggcaccattgatattattgtttgaaacgggtcttgaaggaagtgagaaatctttagttgactggacacctggaagttgacttgattttgggatgtttggtttcaaggaggaatcttttccactgaaaatatccgcgggtataagtgttccaggggctgccattagatccactagaaactcactttcattatccaacttgataatgttgactgcatcatcctcaacaccagt

SEQ ID NO: 285 - RNA, 307 nt, synthetic construct
  source 1..307: mol_type = other RNA
gagtacctgcatattcacctgcttgataagttgtttctaaacattctttagccctatgaacttttgactgtagataaaaactcccattcttggcgttcctttcaaataaattgtcatatttttgtgcgatttcttcccatgatgattgttcattgacaccaagaatttgtctggcttcttgttcagtcatagctttgcttcctttacgcacaacattttgtaatgtgtcttgagcaacacctgattttgatgcattttggagggcttgtctatatgcttgaacaagagccctagccattattccaga

SEQ ID NO: 286 - RNA, 284 nt, synthetic construct
  source 1..284: mol_type = other RNA
tttgactgcaaataaaaactcccattcttcgcatttctctcaaatagattatcatatttctgtgcaatctcctcccaagatgattgttcagtcacaccaagaatttgtctcgcttcttgttcagtcatagctttgcttcctcttcgcacagcattctgtaatgtttcttgagcaacccctgactttgaggcattttggagagcctgacgatatgcttgaaccattcccctaatcaagattccagagcccattacaatcaaatttgcaagaatcttagcagccat

SEQ ID NO: 287 - RNA, 318 nt, synthetic construct
  source 1..318: mol_type = other RNA
aacttgttgtgtgttgttatcttgagggatttggttaacattactagacatagatacaggctcttccatggctactctagatttggtctcttctacttgagatgtttccatttcgacctcgttgactttggtcaaatcagtttcaggttcctttggctgctgctgttggggtttagatactgttttagccatgtcagacgattgtgactttgaagacttctgtttctttgacttcttttcagtggcttttttcacagtttcaacaggctttacttctttcacaacactggactcattagtagatggtttctggacctt

SEQ ID NO: 288 - RNA, 316 nt, synthetic construct
  source 1..316: mol_type = other RNA
aatcacatatttttaaatcacaatttgcattcaataaaagattgcttggctttaaatccctgtgtaacacatttgcagagtgtatgtactttaacccacgaaggatttgatacagaaaatactggcaatgttcctctgataaagattggttagaacggattatttggtggagatcagtgtccatgagctcataagcaatgtagacatcattgaaggatgttctttctggaggtgggattatatctctaattgcaacaacgttttcatgatccatatgacggagaagcttgatttctcgcagtgtcctcttcgcatc

SEQ ID NO: 289 - RNA, 300 nt, synthetic construct
  source 1..300: mol_type = other RNA
atcgaacagtttggatacgtgcacttgtaatagcttctaggattttcacttcctttcacctgcttttgcccgtattttctccaattatacccatcatctgattttttctgcgatggttgattgttcaaattgttgttgctggctgcataaatttcctttgtaaacgatttccgtgatatgtccttctaagtttctttcaactttcttctttgttgggcaattagggaacgtacatttgtaataacttctagggttttcgcttcctttgacttgtttttgaccgtattttctccaattgta

SEQ ID NO: 290 - RNA, 300 nt, synthetic construct
  source 1..300: mol_type = other RNA
tatattttagaattcccacttccaagagtgagttcaagatcatccactccaacatcatgaatcctctcgccttcccatgccttcactcctccattctcaatatatttgagcattcccacttccaagggtgagctcaagatcatcaaaaccgacttcatgaaacctctcgcctttccatgccttcacccctttacccttttaaacgttgatatctagttgggcttgtgggagcagaagatgcaaagtaggggagattgaaagagggaattgactgtttggtgaatgattcccagattagat

(19) United States
(12) Patent Application Publication
(10) Pub. No.: US 2026/0262657 A1
(43) Pub. Date: Sep. 10, 2026

(54) PROTECTION OF PLANTS AGAINST PATHOGENS BY INDUCTION OF SYSTEMIC OR LOCALIZED PROTECTIVE RESPONSES IN PLANTS

(71) Applicant: GreenLight Biosciences, Inc., Medford, MA (US)

(72) Inventors: Bridey B. Maxwell, Medford, MA (US); Maris Ranae Durako, Medford, MA (US); Justin Eldridge, Medford, MA (US); Ronald D. Flannagan, Medford, MA (US); Dale T. Karlson, Medford, MA (US); Wimalanathan Kokulapalan, Medford, MA (US); Jerome Laisney, Medford, MA (US); William Stewart, Medford, MA (US); Reetu Tuteja, Medford, MA (US); Shujian Zhang, Medford, MA (US); Balaji Vasudevan, Medford, MA (US); Alishka Garg, Medford, MA (US)

(73) Assignee: GreenLight Biosciences, Inc., Medford, MA (US)

(21) Appl. No.: 19/162,231

(22) PCT Filed: Mar. 7, 2024
(86) PCT No.: PCT/US2024/018948

§ 371 Date: Sep. 4, 2025

Related U.S. Application Data
(60) Provisional application No. 63/488,962, filed on Mar. 7, 2023.

(51) Int. Cl. A01N 63/60; A01P 21/00; C12N 15/113; C12N 15/82

(52) U.S. Cl. CPC A01N 63/60; A01P 21/00; C12N 15/113; C12N 15/8282; C12N 2310/14

(57) ABSTRACT

Provided herein are compositions of RNAi molecules for targeting a plant gene involved in suppression of a systemic or localized protective response pathway for controlling pathogens (e.g., a fungal pathogen belonging to the genus Fusarium or Phytophthora’) and associated methods of using same.

### RELATED APPLICATIONS

This application claims priority under 35 U.S.C. § 119(e) to U.S. Provisional Application, U.S. Ser. No. 63/488,962, filed Mar. 7, 2023, which is incorporated herein by reference.

### REFERENCE TO AN ELECTRONIC SEQUENCE LISTING

The contents of the electronic sequence listing (G083070045WO00-SEQ-MSB.xml; Size: 534,002 bytes; and Date of Creation: Mar. 6, 2024) is herein incorporated by reference in its entirety.

### BACKGROUND

Plant crops are a target for pathogen (fungal, bacterial, etc.) attacks. Globally, farmers lose 30 to 40 percent of their crops due to pests and diseases, according to the UN Food and Agricultural Organization. Crop maintenance and crop health are essential for yield and quality of produce, which ultimately require long-term strategies for the minimization of pest and disease occurrence. The annual costs of controlling crop pathogenic pests are estimated to be in the tens of millions of dollars, with projected annual costs of crop loss reaching billions of dollars if left uncontrolled. The most prevalent current approach to pest protection is to apply a chemical to a crop that binds to an essential protein in a pest and thus impairing the ability of the pest (e.g., killing the pest or reducing sporulation, growth, or fecundity) to damage the crop. These chemical fungicides can often bind to similar proteins in beneficial organisms in a field and cause collateral damage both locally where the peptide or other fungicide is sprayed and remotely with residual peptide or other fungicide flowing into waterways or spread by air currents to other locations. These fungicides may also have various levels of toxicity to humans.

### SUMMARY

The present disclosure provides, in some aspects, compositions, polynucleotides, genetic constructs, uses, and methods for controlling infection of plants by various pathogens (e.g., pathogens that cause damage to crop plants) through inhibition of a plant gene involved in the suppression of a systemic or localized protective response(s) to a pathogen. These inventions represent a more targeted and more environmentally friendly approach than standard pathogen protection approaches. For example, aspects of the present disclosure provide a polynucleotide (e.g., an isolated polynucleotide) that inhibits a plant gene involved in suppression of a systemic or localized response(s) to a pathogen. In some embodiments, the plant gene is involved in inhibition of a systemic acquired resistance (SAR) pathway. In some embodiments, the plant gene is involved in suppression of a systemic or localized protective response to a fungal pathogen.

The plant gene targeted in the present invention may be selected from the group consisting of MPK4, CPR5, MPK4a, RLK3, NPR3, RFC3, SNI1, CEV1, COI1, RAP, DMR6-like, ARR9, NPR4, NAC25, BRI1a, BRI1b, TAR2, YUC2a, ETR1, EIN4, ETR2, ACS1, CRY1b, CRY2, ACO4, CTR1, MPK6, RIN4b, RIN4a, RapTOR, TOR, GID1B, GID1C, DHS, GA3OX, HOS15, eIF_5A, CLH2, SPL12l, EIN5, WRKY33, WRKY11, BZR1, EDR1, EXA1, WRKY40, JAZ7, PW220, DMR6, RPLK3, JAZ, and PAM16.

The plant gene targeted in the present invention may be selected from the group consisting of MPK4 (Mitogen-activated protein kinase 4), CPR5 (Constitutive Expression of PR Genes 5), MPK4a (Mitogen-activated protein kinase 4a, RLK3 (Receptor-like kinase/leucine-rich repeat receptor-like kinase), NPR3 (NPR-1 like protein 3/salicylic acid receptor), RFC3 (Replication factor C subunit 3), SNI1 (Suppressor of NPR-1 1), CEV1 (constitutive expression of VSP 1/cellulose synthase family protein), COI1 (coronatine-insensitive 1), RAP (RNA-binding domain), DMR6 (Downy mildew Resistance 6), DMR6-like (Downy mildew Resistance 6-like), ARR9 (response regulator 9/two-component response regulator-like protein), NPR4 (NPR1-like protein 4/salicylic acid receptor), NAC25 (encoding NAC domain containing protein 25), BRIla (brassinosteroid insensitive 1a/Leucine-rich receptor-like protein kinase), BRIlb (brassinosteroid insensitive 1b/Leucine-rich receptor-like protein kinase), TAR2 (tryptophan aminotransferase related 2), YUC2a (Yucca/indole-3-pyruvate monooxygenase), ETR1 (ethylene response 1/ethylene receptor histidine kinase), EIN4 (ethylene insensitive 4/ethylene receptor histidine kinase), ETR2 (ethylene response 2/ethylene receptor histidine kinase), ACS1 (ACC synthase 1/enzymatically inactive form of 1-aminocyclopropane-1-carboxylate synthase), CRY1b (Cryptochrome 1/flavin-type blue-light photoreceptor), CRY2 (Cryptochrome 2/flavin-type blue-light photoreceptor), ACO4 (1-aminocyclopropane-1-carboxylic acid oxidase 4), CTR1 (Constitutive triple response 1/serine/threonine protein kinase), MPK6 (Mitogen-activated protein kinase 6), RIN4b (RPM1 interacting protein 4b), RIN4a (RPM1 interacting protein 4), RapTOR (Regulatory Associated Protein Of TOR), TOR (encoding target of rapamycin), GID1B (GA Insensitive Dwarf 1B/Gibberellin receptor, GID1C (GA Insensitive Dwarf 1C/Gibberellin receptor), DHS (deoxyhypusine synthase), GA3OX (Gibberellin 3-oxidase), HOS15 (high expression of osmotically responsive genes 15/WD-40 repeat protein), eIF_5A (eukaryotic translation initiation factor 5A), CLH2 (chlorophyllase 2), SPL12l (Squamosa promoter-binding-like protein 12), EIN5 (ethylene insensitive 5/5′→3′ exoribonuclease), WRKY33 (WRKY DNA-binding protein 33/DNA-binding transcription factor), WRKY11 (WRKY DNA-binding protein 11/DNA-binding transcription factor), BZR1 (Brassinazole resistant 1/DNA-binding transcriptional repressor), EDR1 (enhanced disease resistance 1/protein kinase), EXA1 (essential for potexvirus accumulation 1), WRKY40 (WRKY DNA-binding protein 40/DNA-binding transcription factor), JAZ7 (encoding jasmonate-zim-domain protein 7), PW220 (JAZ10/Promoter wound inducible 220/jasmonate-zim-domain protein 10), RPLK3 (receptor-like protein kinase 3-like/Leucine-rich receptor-like protein kinase family protein/Ortholog of AT5G65700), JAZ (jasmonate-zim-domain protein), and PAM16 (presequence translocase-associated motor 16).

The plant gene may be a MPK4a, RLK3, RIN4a, CEV1, CPR5, COI1, DHS, ETR1, EIN4, ETR2, or WRKY40 gene. In some embodiments, the plant gene is a CPR5 gene.

The plant gene may be derived from Arabidopsis thaliana (At), Glycine max (Gm), Lactuca sativa (Ls), Nicotiana benthamiana (Nb), Solanum lycopersicum (Sl), or Spinacia oleracea (So). The plant gene may be derived from Solanaceae plants, Brassicaceae plants, Poaceae plants, Cucurbitaceae plants, Fobaceae plants, Apiaceae plants, Amaranthaceae plants, or Malvaceae plants.

In some embodiments, the plant gene is selected from the group consisting of: AtMPK4, AtCPR5, GmMPK4a, GmRLK3, GmCPR5, GmNPR3, GmRFC3, GmSNI1, GmCEV1, GmCOI1, GmRAP, GmDMR6-like, GmARR9, GmNPR4, GmNAC25, GmBRI1a, GmBRI1b, GmTAR2, GmYUC2a, GmETR1, GmEIN4, GmETR2, GmACS1, GmCRY1b, GmCRY2, GmACO4, GmCTR1, GmMPK6, GmRIN4b, GmRIN4a, GmRapTOR, GmTOR, GmGID1B, GmGID1C, GmDHS, GmGA3OX, GmHOS15, GmeIF_5A, GmCLH2, GmSPL12l, LsCPR5, LsCPR5, NbEIN5, SlEIN5, SlCPR5, SoWRKY33, SoWRKY11, SoHOS15, SoBZR1, SoEDR1, SoEXA1, SoWRKY40, SoJAZ7, SoPW220, SoMPK6, SoSPL12I, SoCEV1, SoCPR5, SoCOI1, SoDMR6, SoDHS, SoelF-5A, SoETR1, SoEIN4, SoETR2, SoCTR1, SoMPK4a, SoRPLK3, SoBZR1, SoWRKY40, SoJAZ, SoDHS, SomelF5-A, SoMPK4a, SoETR1, SoEIN4, SoETR2, SoCEV1, and SoCPR5.

In some embodiments, the polynucleotide (e.g., isolated polynucleotide) is a ribonucleic acid (RNA). In some embodiments, the polynucleotide (e.g., isolated polynucleotide) is a deoxyribonucleic acid (DNA). In some embodiments, the polynucleotide is a dsRNA. In some embodiments, the RNA is a double-stranded RNA (dsRNA) comprising a first strand that is complementary to a segment of the coding region of a messenger RNA (mRNA) encoded by the gene, and a second strand that is complementary to the first strand. In some embodiments, the polynucleotide is a single-stranded RNA (ssRNA).

In some embodiments, the gene comprises a nucleic acid sequence having at least 80%, at least 85%, at least 90%, at least 95%, at least 97%, at least 98%, at least 99%, or 100% identity to any one of SEQ ID NOs: 1-86 or 265-274. In some embodiments, the polynucleotide (e.g., isolated polynucleotide) is complementary to a segment of a plant gene (e.g., a plant gene comprising a nucleic acid sequence having at least 80%, at least 85%, at least 90%, at least 95%, at least 97%, at least 98%, at least 99%, or 100% identity to any one of SEQ ID NOs: 1-86 or 265-274). In some embodiments, the polynucleotide (e.g., isolated polynucleotide) is complementary to a segment of a plant gene (e.g., a plant gene comprising a nucleic acid sequence having at least 80%, at least 85%, at least 90%, at least 95%, at least 97%, at least 98%, at least 99%, or 100% identity to any one of SEQ ID NOs: 1-86 or 265-274), wherein the segment of the plant gene comprises at least 15, at least 20, at least 25, at least 30, at least 40, at least 50, at least 60, at least 70, at least 80, at least 90, or at least 100 contiguous nucleotides of the plant gene. In some embodiments, the polynucleotide (e.g., isolated polynucleotide) comprises a nucleic acid sequence identical or complementary (e.g., perfectly complementary) to at least 18, 19, 20, 21, 22, 23, 24, 25, 26, 27, 28, 29, or 30 contiguous nucleotides. In some embodiments, the polynucleotide is at least 70%, 80%, 85%, 90%, 95%, or 100% identical or complementary to at least 21 contiguous nucleotides of the plant gene.

In some embodiments, the polynucleotide (e.g., isolated polynucleotide) comprises a strand with a nucleic acid sequence having at least 70% identity to any one of SEQ ID NOs: 87-258 and 275-290. In some embodiments, the polynucleotide comprises a nucleic acid sequence having at least 80%, at least 85%, at least 90%, at least 95%, at least 97%, at least 98%, at least 99%, or 100% identity to any one of SEQ ID NOs: 87-258 and 275-290. In some embodiments, the polynucleotide comprises at least 15, at least 20, at least 25, at least 30, at least 40, at least 50, at least 60, at least 70, at least 80, at least 90, or at least 100 contiguous nucleotides having at least 80%, at least 85%, at least 90%, at least 95%, at least 97%, at least 98%, at least 99%, or 100% identity to a corresponding segment of any one of SEQ ID NOs: 87-258 and 275-290.

In some embodiments, the polynucleotide (e.g., isolated polynucleotide) comprises a strand with a nucleic acid sequence having at least 70% identity to any one of SEQ ID NOs: 173-258 or 283-290. In some embodiments, the polynucleotide comprises a nucleic acid sequence having at least 80%, at least 85%, at least 90%, at least 95%, at least 97%, at least 98%, at least 99%, or 100% identity to any one of SEQ ID NOs: 87-172 or 275-282.

In some embodiments, the polynucleotide is a dsRNA comprising a second strand complementary to the first strand. In some embodiments, the polynucleotide is a dsRNA comprising (a) a first strand having at least 70%, at least 80%, at least 85%, at least 90%, at least 95%, at least 97%, at least 98%, at least 99%, or 100% identity to any one of SEQ ID NOs: 87-172 or 275-282; and (b) a first strand having at least 70%, at least 80%, at least 85%, at least 90%, at least 95%, at least 97%, at least 98%, at least 99%, or 100% identity to any one of SEQ ID NOs: 173-258 or 283-290.

In some embodiments, the polynucleotide comprises at least 18, 19, 20, 21, 25, 50, 100, 150, 200, 250, 300, 400, or 450 contiguous nucleotides that are complementary to or comprise at least about 85%, at least 90%, at least 95%, at least about 98%, or about 100% sequence identity to with a segment of a DNA or target gene having a nucleotide sequence selected from the group consisting of SEQ ID NOs: 1-86 or 265-274. In some embodiments, the polynucleotide comprises at least 18, 19, 20, 21, 25, 50, 100, 150, 200, 250, 300, 350, 400, or 450 contiguous nucleotides that are complementary to, or comprise at least about 85%, at least about 90%, at least about 95%, at least about 98%, about 100%, or 100% sequence identity with a segment of a sequence selected from the group consisting of: SEQ ID NOs: 87-258 or 275-290.

In some embodiments, the polynucleotide is a dsRNA comprising a first strand comprising at least 18, 19, 20, 21, 25, 50, 100, 150, 200, 250, 300, 350, 400, or 450 contiguous nucleotides that are complementary to, or comprises at least about 85%, at least about 90%, at least about 95%, at least about 98%, about 100%, or 100% sequence identity with a segment of a sequence selected from the group consisting of SEQ ID NOs: 87-172, 275-282; further comprising a second strand complementary to the first, the second strand comprising 18, 19, 20, 21, 25, 50, 100, 150, 200, 250, 300, 350, 400, 450, or 500 contiguous nucleotides that are complementary to, or comprises at least about 85%, at least about 90%, at least about 95%, at least about 98%, about 100%, or 100% sequence identity with a segment of a corresponding reverse complement sequence selected from the group consisting of SEQ ID NOs: 173-258, 283-290.

In some embodiments, the polynucleotide comprises at least about 200 contiguous nucleotides that are complementary or comprise at least about 90% identity with a segment of a sequence selected from the group consisting of SEQ ID NOs: 87-258 or 275-290.

In some embodiments, the DNA or target gene has a nucleotide sequence selected from the group consisting of SEQ ID NOs: 2, 5, 35, 36, 41, 42, 62, 85, and 86. In some embodiments, the polynucleotide comprises at least 18, 19, 20, 21, 25, 50, 100, 150, 200, 250, 300, 350, 400, 450, or 500 contiguous nucleotides that are complementary to, or comprise at least about 85%, at least about 90%, at least about 95%, at least about 98%, about 100%, or 100% sequence identity with a segment of a sequence selected from the group consisting of: SEQ ID NOs: 88, 91, 121, 122, 127, 128, 148, 171, 172, and 174, 177, 207, 208, 213, 214, 234, 257, and 285. In some embodiments, the polynucleotide comprises at least about 200 contiguous nucleotides that are complementary or comprise at least about 90% identity with a segment of a sequence selected from the group consisting of SEQ ID NOs: 88, 91, 121, 122, 127, 128, 148, 171, 172, 174, 177, 207, 208, 213, 214, 234, 257, and 285.

In some embodiments, the DNA or target gene has a nucleotide sequence selected from the group consisting of SEQ ID NOs: 3, 74, 75, 76, and 82. In some embodiments, the polynucleotide comprises at least 18, 19, 20, 21, 25, 50, 100, 150, 200, 250, 300, 350, 400, 450, or 500 contiguous nucleotides that are complementary to, or comprise at least about 85%, at least about 90%, at least about 95%, at least about 98%, about 100%, or 100% sequence identity with a segment of a sequence selected from the group consisting of: SEQ ID NOs: 89, 160, 161, 162, 168, 175, 246, 247, 248, and 254. In some embodiments, the polynucleotide comprises at least about 200 contiguous nucleotides that are complementary or comprise at least about 90% identity with a segment of a sequence selected from the group consisting of SEQ ID NOs: 89, 160, 161, 162, 168, 175, 246, 247, 248, and 254.

In some embodiments, the DNA or target gene has a nucleotide sequence of SEQ ID NO: 4. In some embodiments, the polynucleotide comprises at least 18, 19, 20, 21, 25, 50, 100, 150, 200, 250, 300, 350, 400, 450, or 500 contiguous nucleotides that are complementary to, or comprise at least about 85%, at least about 90%, at least about 95%, at least about 98%, about 100%, or 100% sequence identity with a segment of a sequence selected from the group consisting of: SEQ ID NOs: 90 and 176. In some embodiments, the polynucleotide comprises at least about 200 contiguous nucleotides that are complementary or comprise at least about 90% identity with a segment of a sequence selected from the group consisting of SEQ ID NOs: 90 and 176.

In some embodiments, the DNA or target gene has a nucleotide sequence of SEQ ID NO: 25. In some embodiments, the polynucleotide comprises at least 18, 19, 20, 21, 25, 50, 100, 150, 200, 250, 300, 350, 400, 450, or 500 contiguous nucleotides that are complementary to, or comprise at least about 85%, at least about 90%, at least about 95%, at least about 98%, about 100%, or 100% sequence identity with a segment of a sequence selected from the group consisting of: SEQ ID NOs: 111 and 197. In some embodiments, the polynucleotide comprises at least about 200 contiguous nucleotides that are complementary or comprise at least about 90% identity with a segment of a sequence selected from the group consisting of SEQ ID NOs: 111 and 197.

In some embodiments, the DNA or target gene has a nucleotide sequence selected from the group consisting of SEQ ID NOs: 9, 59, 60, 61, and 84. In some embodiments, the polynucleotide comprises at least 18, 19, 20, 21, 25, 50, 100, 150, 200, 250, 300, 350, 400, 450, or 500 contiguous nucleotides that are complementary to, or comprise at least about 85%, at least about 90%, at least about 95%, at least about 98%, about 100%, or 100% sequence identity with a segment of a sequence selected from the group consisting of: SEQ ID NOs: 95, 181, 145, 231, 146, 232, 147, 233, 170, and 256. In some embodiments, the polynucleotide comprises at least about 200 contiguous nucleotides that are complementary or comprise at least about 90% identity with a segment of a sequence selected from the group consisting of SEQ ID NOs: 95, 181, 145, 231, 146, 232, 147, 233, 170, and 256.

In some embodiments, the DNA or target gene has a nucleotide sequence selected from the group consisting of SEQ ID NOs: 10 and 63. In some embodiments, the polynucleotide comprises at least 18, 19, 20, 21, 25, 50, 100, 150, 200, 250, 300, 350, 400, 450, or 500 contiguous nucleotides that are complementary to, or comprise at least about 85%, at least about 90%, at least about 95%, at least about 98%, about 100%, or 100% sequence identity with a segment of a sequence selected from the group consisting of: SEQ ID NOs: 96, 182, 149, and 235. In some embodiments, the polynucleotide comprises at least about 200 contiguous nucleotides that are complementary or comprise at least about 90% identity with a segment of a sequence selected from the group consisting of SEQ ID NOs: 96, 182, 149, and 235.

In some embodiments, the DNA or target gene has a nucleotide sequence selected from the group consisting of SEQ ID NOs: 29, 65, 66, and 80. In some embodiments, the polynucleotide comprises at least 18, 19, 20, 21, 25, 50, 100, 150, 200, 250, 300, 350, 400, 450, or 500 contiguous nucleotides that are complementary to, or comprise at least about 85%, at least about 90%, at least about 95%, at least about 98%, about 100%, or 100% sequence identity with a segment of a sequence selected from the group consisting of: SEQ ID NOs: 115, 201, 151, 237, 152, 238, 166, and 252. In some embodiments, the polynucleotide comprises at least about 200 contiguous nucleotides that are complementary or comprise at least about 90% identity with a segment of a sequence selected from the group consisting of SEQ ID NOs: 115, 201, 151, 237, 152, 238, 166, and 252.

In some embodiments, the DNA or target gene has a nucleotide sequence selected from the group consisting of SEQ ID NOs: 18 and 83. In some embodiments, the polynucleotide comprises at least 18, 19, 20, 21, 25, 50, 100, 150, 200, 250, 300, 350, 400, 450, or 500 contiguous nucleotides that are complementary to, or comprise at least about 85%, at least about 90%, at least about 95%, at least about 98%, about 100%, or 100% sequence identity with a segment of a sequence selected from the group consisting of: SEQ ID NOs: 104, 190, 169, and 255. In some embodiments, the polynucleotide comprises at least about 200 contiguous nucleotides that are complementary or comprise at least about 90% identity with a segment of a sequence selected from the group consisting of SEQ ID NOs: 104, 190, 169, and 255.

In some embodiments, the DNA or target gene has a nucleotide sequence selected from the group consisting of SEQ ID NOs: 52, 53, and 78. In some embodiments, the polynucleotide comprises at least 18, 19, 20, 21, 25, 50, 100, 150, 200, 250, 300, 350, 400, 450, or 500 contiguous nucleotides that are complementary to, or comprise at least about 85%, at least about 90%, at least about 95%, at least about 98%, about 100%, or 100% sequence identity with a segment of a sequence selected from the group consisting of: SEQ ID NOs: 138, 224, 139, 225, 164, and 250. In some embodiments, the polynucleotide comprises at least about 200 contiguous nucleotides that are complementary or comprise at least about 90% identity with a segment of a sequence selected from the group consisting of SEQ ID NOs: 138, 224, 139, 225, 164, and 250. In some embodiments, the polynucleotide (e.g., isolated polynucleotide) has a length of at least 18 to 600 nucleotides, at least 21 to 600 nucleotides, at least 50 to 600 nucleotides, at least 100 to 500 nucleotides, at least 200 to 500 nucleotides, or at least 300 to 450 nucleotides. In some embodiments, the polynucleotide (e.g., isolated polynucleotide) has a length of 18 to 500 nucleotides, 21 to 500 nucleotides, 50 to 500 nucleotides, 100 to 500 nucleotides, 200 to 500 nucleotides, or 300 to 450 nucleotides. In some embodiments, the polynucleotide (e.g., isolated polynucleotide) has a length of at least 18 nucleotides, at least 21 nucleotides, at least 50 nucleotides, at least 100 nucleotides, at least 200 nucleotides, or at least 300 nucleotides. The polynucleotide may inhibit one, two, three or four independent plant genes and their paralogs involved in suppression of a systemic or localized responses to a pathogen. The polynucleotide may be a chimeric polynucleotide comprising at least two different nucleic acid sequences, wherein each of the at least two different nucleic acid sequences inhibits a different plant gene involved in suppression of a systemic or localized responses to a pathogen.

In some embodiments, the polynucleotide comprises a first nucleic acid that inhibits ETR1, a second nucleic acid that inhibits EIN4, and a third nucleic acid that inhibits ETR2.

Some aspects of the present disclosure provide a composition comprising a polynucleotide described herein. In some embodiments, the composition further comprises a carbon quantum dot. In some embodiments, the composition further comprises polyethylene glycol (PEG) (e.g., PEG 200). In some embodiments the composition further comprises one or more chemical fungicides and/or biofungicides. In some embodiments the use of the polynucleotides of the present invention allows for a lower dose of a chemical fungicide or biofungicide without a loss in efficacy of the chemical fungicide or biofungicide.

In some embodiments, the composition further comprises a ligand. The ligand may be polyethylene glycol (e.g., PEG 200, PEG 1000, PEG 2000, PEG 5000, or PEG 10000), a carbohydrate, a porphyrin conjugate, peptide, or lipid. In some embodiments the composition further comprises a surfactant. In some embodiments the surfactant is Pluronic F-127.

Some aspects of the present disclosure provide a method of increasing resistance to a pathogen in a plant comprising activating a systemic or localized protective response to a pathogen of the plant prior to exposure of the plant to the pathogen.

Some aspects of the present disclosure provide a method of increasing resistance to a pathogen in a plant comprising increasing the activity of a systemic or localized protective response to a pathogen.

Some aspects of the present disclosure provide a method of increasing the lifespan of a plant, or the average lifespan of a population of plants or increase in the biomass of a population of plants or increase in the yield of a population of plants, comprising activating a systemic protective response to a pathogen prior to exposure of the of the plant or the population of plants to the pathogen.

Activating the systemic protective response may comprise inhibiting in the plant one or more plant genes involved in suppression of the systemic response, optionally wherein the one or more plant genes systemic protective responses to fungal pathogens or other microbes, optionally wherein the gene is involved in inhibition of a systemic acquired resistance (SAR) pathway.

In some embodiments, inhibiting one or more plant genes involved in suppression of a systemic or localized protective responses further results in increased expression of a gene or set of genes involved in triggering or carrying out a systemic or localized protective response. In some embodiments the gene involved in triggering or carrying out a systemic or localized protective response encodes an anti-fungal protein. In some embodiments the gene involved triggering or carrying out a systemic or localized protective response encodes a transcription factor. In some embodiments the gene involved in triggering or carrying out a systemic or localized protective response encodes a plant hormone biosynthesis enzyme. In some embodiments the gene involved in triggering or carrying out a systemic or localized protective response encodes a plant hormone receptor. In some embodiments the gene involved in triggering or carrying out a systemic or localized protective response encodes a transcription factor. In some embodiments the gene involved in triggering or carrying out a systemic or localized protective response encodes a phosphorelay signaling protein. In some embodiments the gene involved in triggering or carrying out a systemic or localized protective response encodes a biosynthetic enzyme responsible for synthesis of one or more antifungal metabolites. In some embodiments the gene involved in triggering or carrying out a systemic or localized protective response encodes a cell wall incorporated protein or metabolite responsible for physically preventing spread of the pathogen. In some embodiments the gene involved in triggering or carrying out a systemic or localized protective response encodes a plant cell wall component biosynthesis enzyme. In some embodiments the gene involved in triggering or carrying out a systemic or localized protective response encodes a fungal cell wall digestion enzyme. In some embodiments the gene involved in triggering or carrying out a systemic or localized protective responses comprise one or more of PR1, PR3, PR5, HARB, ZF, GT61 and bHLH.

In some embodiments, inhibiting one or more plant genes involved in suppression of a systemic or localized protective response comprises delivering to the plant a polynucleotide or a protein that inhibits expression of the one or more plant genes.

In some embodiments, the polynucleotide that inhibits expression functions through an RNA interference (RNAi) mechanism. In some embodiments, the one or more plant genes are selected from the group consisting of: MPK4, CPR5, MPK4a, RLK3, NPR3, RFC3, SNI1, CEV1, COI1, RAP, DMR6-like, ARR9, NPR4, NAC25, BRI1a, BRI1b, TAR2, YUC2a, ETR1, EIN4, ETR2, ACS1, CRY1b, CRY2, ACO4, CTR1, MPK6, RIN4b, RIN4a, RapTOR, TOR, GID1B, GID1C, DHS, GA3OX, HOS15, eIF_5A, CLH2, SPL12l, EIN5, WRKY33, WRKY11, BZR1, EDR1, EXA1, WRKY40, JAZ7, PW220, DMR6, RPLK3, JAZ, and PAM16. In some embodiments, the one or more plant genes are selected from the group consisting of: AtMPK4, AtCPR5, GmMPK4a, GmRLK3, GmCPR5, GmNPR3, GmRFC3, GmSNI1, GmCEV1, GmCOI1, GmRAP, GmDMR6-like, GmARR9, GmNPR4, GmNAC25, GmBRI1a, GmBRI1b, GmTAR2, GmYUC2a, GmETR1, EIN4, ETR2, GmACS1, GmCRY1b, GmCRY2, GmACO4, GmCTR1, GmMPK6, GmRIN4b, GmRIN4a, GmRapTOR, GmTOR, GmGID1B, GmGID1C, GmDHS, GmGA3OX, GmHOS15, GmeIF_5A, GmCLH2, GmSPL12l, LsCPR5, LsCPR5, LsHOS15, LsEDR1, LsPAM16, LsEXA1, LsMPK6, LsWRKY33, LsBZR1, NbEIN5, SlEIN5, SlCPR5, SoWRKY33, SoWRKY11, SoHOS15, SoBZR1, SoEDR1, SoEXA1, SoWRKY40, SoJAZ7, SoPW220, SoMPK6, SoSPL12I, SoCEV1, SoCPR5, SoCOI1, SoDMR6, SoDHS, SomelF-5A, SoETR1, SoEIN4, SoETR2, SoCTR1, SoMPK4a, SoRPLK3, SoBZR1, SoWRKY40, SoJAZ, SoDHS, SomelF5-A, SoMPK4a, SoETR1, SEIN4, SoETR2, SoCEV1, and SoCPR5.

In some embodiments, inhibiting one or more plant genes comprises delivering to the plant one or more of the polynucleotides described herein or one or more of the compositions described herein.

In some embodiments, the polynucleotide is delivered to the plant at the seedling or seed stage of the plant lifecycle. In some embodiments, the polynucleotide is delivered to the leaves or roots of the plant. In some embodiments, the polynucleotide is delivered to the seed of the plant, optionally wherein the polynucleotide is delivered through soaking the seed of the plant.

In some embodiments, the polynucleotide comprises a nucleic acid sequence that is complementary to paralogous, homologous or functionally redundant plant genes involved in suppression of a systemic or localized protective response. In some embodiments, the homologous, paralogous or functionally redundant plant genes are from the same species.

The plant being treated in the present invention may be any plant. In some embodiments, the plant is selected from the group consisting of Asteraceae plants, Solanaceae plants, Brassicaceae plants, Poaceae plants, Cucurbitaceae plants, Fabaceae plants, Apiaceae plants, Amaranthaceae plants, Malvaceae plants, Actinidiaceae plants, Amaryllidaceae plants, Anacardiaceae plants, Annonaceae plants, Boraginaceae plants, Bromeliaceae plants, Chenopodiaceae plants, Convolvulaceae plants, Ebenaceae plants, Ericaceae plants, Fabaceae or Leguminosae plants, Grossulariaceae plants, Lamiaceae plants, Lauraceae plants, Lythraceae plants, Malvaceae plants, Moraceae plants, Musaceae plants, Myrthaceae plants, Oleaceae plants, Passifloraceae plants, Polygonaceae plants, Rosaceae plants, Rutaceae plants, Sapindaceae plants, Verbenaceae plants, Vitaceae plants, and Zingiberaceae plants. In some embodiments, the plant is a vegetable, fruit, grain, or legume. In some embodiments, the plant is a row crop, flowering plant, or tree. In some embodiments, the plant is oil crops, vegetables, pulses, fiber crops, ornamentals, and spices. The plant may be corn, cereal, cotton, fruit, tree nut, rice, or soybean. In some embodiments, the plant is soy, spinach, lettuce, cauliflower, pea shoots, rice, or wheat.

In some embodiments, the pathogen is a bacterial pathogen, a viral pathogen, or a fungal pathogen. In some embodiments, the pathogen is a fungal pathogen. In some embodiments, the fungal pathogen belongs to the genus Fusarium, the genus Phytophthora, the genus Rhizoctonia, the genus Macrophomina, the genus Septoria, the genus Ptyhium, the genus Sclerotina, the genus Colletotrichum, the genus Cladosporium, the genus Peronospora, the genus Stemphylium, the genus Bremia, or the genus Erysiphe. In some embodiments the pathogen is a bacterial pathogen. In some embodiments the bacterial pathogen belongs to the genus Pseudomonas or the genus Erwinia. In some embodiments the pathogen is a virus. In some embodiments the virus is Beet curly top virus (BCTV), Beet western yellows virus (BWYV), Cucumber mosaic virus (CMV), Impatiens necrotic spot virus (INSV), Tobacco rattle virus (TRV), Tomato spotted wilt virus (TSWV), Lettuce Infectious Yellows Virus (LIYV), Lettuce Mosaic Virus (LMV), Beet Western Yellows Virus (BWYV), Cucumber Mosaic Virus (CMV), Alfalfa Mosaic Virus (AMV), a Big Vein Virus, Tomato spotted wilt virus, Impatiens necrotic spot virus, Lettuce necrotic stunt virus, and Tomato bushy stunt virus.

The pathogen may be a Fusarium spp., Phakospora spp., Rhizoctonia spp., Aspergillus spp., Gibberella spp., Pyricularia spp., Alternaria spp., and Phytophthora spp. In some embodiments, the pathogen is Rhizoctonia solani.

In some embodiments, the systemic or localized protective response pathway is a systemic acquired resistance (SAR) pathway.

The method may further comprises determining an expression level of one or more systemic or localized protective response pathway genes. An expression level of a PR gene (e.g. PR1, PR3, or PR5 gene may be determined. An expression level of HARB, ZF, GT61 and bHLH may be determined.

### BRIEF DESCRIPTION OF THE DRAWINGS

FIGS. 1A-1B show that carbon quantum dots can deliver dsRNA to soy plants to confer a visible phenotype (luminescence from the carbon quantum dot) in first true leaves at 12 days after planting.

FIG. 2 shows the delivery of dsRNA targeting systemic acquired resistance (SAR) pathway genes can upregulate the SAR pathway in soybeans.

FIG. 3 shows that delivery of dsRNA targeting SAR pathway genes can protect soybean seedlings from fungal disease.

FIGS. 4A-4B show a dsRNA targeting GmMPK4a (GS200) applied as a seed treatment protected seedlings from Rhizoctonia solani.

FIGS. 5-6 show that a dsRNA targeting GmMPK4a (GS200) increased GmPR1 expression (molecular marker for systemic acquired resistance pathway) in soybean plants.

FIGS. 7-8 show that a dsRNA targeting GmRLK3 (GS201) increased GmPR5 expression in soybean plants.

FIG. 9 shows that a dsRNA targeting GmRIN4a (GS2110) increased GmPR1 expression in soybean plants.

FIG. 10 shows that a dsRNA targeting GmRIN4b (GS2109) increased GmPR1 expression in soybean plants.

FIG. 11 shows that a dsRNA targeting GmCEV1 (GS279) increased GmPR3 expression in soybean plants.

FIG. 12 shows that a dsRNA targeting GmCOI1 (GS280) increased GmPR1 expression in soybean plants.

FIG. 13 shows that a dsRNA targeting GmDHS (GS415) increased GmPR1 expression in soybean plants.

FIG. 14 shows that a chimeric dsRNA targeting GmETR1, GmEIN4, and GmETR2 (GS289) increased GmPR1 expression in soybean plants.

FIG. 15 shows that a dsRNA targeting GmCPR5 (GS275) increased GmPR1 expression in soybean plants.

FIG. 16A show activation of natural plant SAR protection for seedlings.

FIG. 16B show that a dsRNA targeting SoWRKY33 (GS6263) increased the expression of defense-associated genes in spinach seedlings.

FIGS. 17A-17B show that a dsRNA targeting LsCPR5 (GS5461) improved the health of lettuce plants in Fusarium chamber assays.

FIG. 18 shows that a dsRNA targeting LsCPR5 (GS5461) increased lettuce survival under field Fusarium challenge across three lettuce varieties and two field locations.

FIG. 19 shows an exemplary chimeric dsRNA structure for targeting homologous or functionally redundant genes from the same species. For example, homologs A and B (from the same species) are closely related but not 100% identical (<95% identity). A chimeric dsRNA may or may not be a continuous full-length match for both the target genes depending on the level of identity. In the case of low percentage identity, chimeras are designed by stitching the sequences from the target genes and therefore not a full-length match for any target.

FIG. 20 shows the increase in percent of healthy spinach plants when including dsRNA targeting a defense repressor gene (CPR5) as compared to the standard seed priming treatment by day 21 of a chamber-grown Fusarium oxysporum challenge. *p<0.12

FIG. 21 shows the increase in percent of healthy lettuce plants and a 2-fold decrease in plant mortality when including dsRNA (GS5461) targeting LsCPR5 as compared to the standard seed priming treatment by day 35 of a chamber-grown Fusarium oxysporum challenge. *p<0.12, **p<0.02

FIG. 22 shows the increase in an immunity gene LsGT61, panel A, corresponding to a decrease in the GS5461 target gene LsCPR5, panel B, in lettuce leaves *p<0.05.

### DETAILED DESCRIPTION

According to some aspects of the present disclosure, RNA interference (RNAi) molecules (e.g., dsRNAs) targeting a plant gene involved in suppression of a systemic and/or localized protective response pathway (e.g., systemic acquired resistance pathway) are effective at interfering with the mRNA encoded by the gene in plant cells, thereby reducing or eliminating translation of the mRNA (e.g., into its corresponding protein). Inhibition of genes involved in suppression of a systemic and/or localized protective response pathway (e.g., genes such as MPK4, CPR5, MPK4a, RLK3, NPR3, RFC3, SNI1, CEV1, COI1, RAP, DMR6-like, ARR9, NPR4, NAC25, BRI1a, BRI1b, TAR2, YUC2a, ETR1, EIN4, ETR2, ACS1, CRY1b, CRY2, ACO4, CTR1, MPK6, RIN4b, RIN4a, RapTOR, TOR, GID1B, GID1C, DHS, GA3OX, HOS15, eIF_5A, CLH2, SPL12l, EIN5, WRKY33, WRKY11, BZR1, EDR1, EXA1, WRKY40, JAZ7, PW220, DMR6, RPLK3, JAZ, and PAM16) results in activation or increased activity of a systemic and/or localized protective response pathway and thus increases the plant's resistance to pathogens (e.g., fungal pathogens). Accordingly, in some aspects, the present disclosure provides polynucleotides, compositions, uses, and methods for controlling pathogen infections in plants by contacting any portion of a plant (e.g., seeds, roots, tubers, stem, branches, leaves, flowers, etc.) with an RNAi molecule as provided herein. As used herein, an RNAi molecule is an RNA-based molecule that functions through a sequence-specific mechanism to suppress gene expression through translational or transcriptional repression. An RNAi molecule may be a double-stranded or single-stranded nucleic acid. In some embodiments, an RNAi molecule comprises RNA and/or DNA nucleotides (i.e., ribonucleotides and/or deoxyribonucleotides).

Systemic and localized responses by a plant to a pathogen involves a reaction which occurs within the plant for the purpose of defending against exogenous agents such as pathogens (e.g., fungal pathogens). A localized response occurs with the organ of the plant that was attacked, or otherwise threatened, by the pathogen. For example, if a pathogen attacks the root system of a plant, then a localized response by the plant would involve activation of defense pathways and genes within the root system. A systemic response occurs across the entire plant (or at least one organ of the plant that was not originally threatened by the pathogen) when any region or organ of the plant has been threatened by a pathogen.

In some instances, a systemic response involves an enhanced resistance that is referred to as systemic acquired resistance (SAR). In the SAR state, plants are primed (sensitized) to more quickly and more effectively activate defense responses the second time they encounter pathogen attack. SAR is induced by most pathogens that cause tissue necrosis, either as a part of a hypersensitive response (HR) or as a symptom of disease. SAR provides activity against a broad spectrum of pathogens which includes viruses, bacteria, oomycetes, and fungi. In some embodiments, SAR confers a long-lasting protection that can last for weeks to month. A core set of SAR pathway genes (SAR genes) may include genes encoding pathogenesis-related (PR) proteins. Selected PR proteins have been identified as acidic β-1,3-glucanases (BGL2) and chitinases (PR-3). In some embodiments, the plant genes (such as MPK4, CPR5, MPK4a, RLK3, NPR3, RFC3, SNI1, CEV1, COI1, RAP, DMR6-like, ARR9, NPR4, NAC25, BRI1a, BRI1b, TAR2, YUC2a, ETR1, EIN4, ETR2, ACS1, CRY1b, CRY2, ACO4, CTR1, MPK6, RIN4b, RIN4a, RapTOR, TOR, GID1B, GID1C, DHS, GA3OX, HOS15, eIF_5A, CLH2, SPL12l, EIN5, WRKY33, WRKY11, BZR1, EDR1, EXA1, WRKY40, JAZ7, PW220, DMR6, RPLK3, JAZ, and PAM16) designated for targeting using the compositions and methods of the present disclosure suppress (or inhibit) the SAR pathway.

Select methods of the disclosure involve inhibition of one or more plant genes (such as MPK4, CPR5, MPK4a, RLK3, NPR3, RFC3, SNI1, CEV1, COI1, RAP, DMR6-like, ARR9, NPR4, NAC25, BRI1a, BRI1b, TAR2, YUC2a, ETR1, EIN4, ETR2, ACS1, CRY1b, CRY2, ACO4, CTR1, MPK6, RIN4b, RIN4a, RapTOR, TOR, GID1B, GID1C, DHS, GA3OX, HOS15, eIF_5A, CLH2, SPL12l, EIN5, WRKY33, WRKY11, BZR1, EDR1, EXA1, WRKY40, JAZ7, PW220, DMR6, RPLK3, JAZ, and PAM16) followed by a determination of the expression level(s) of one or more SAR genes. In some embodiments, the one or more SAR genes to be observed in such methods include PR genes (e.g., PR1, PR3, PR5).

In some embodiments, methods of the disclosure involve inhibition of a MPK4a, RLK3, RIN4a, CEV1, CPR5, COI1, DHS, ETR1, EIN4, ETR2, or WRKY40 gene. In some embodiments, methods of the disclosure involve inhibition of a CPR5 gene.

Expression of a gene in a cell (e.g., plant cell), for example, is considered to be inhibited or reduced through contact with an RNAi molecule if the level of mRNA and/or protein encoded by the gene is reduced in the cell by at least 5% (e.g., at least 10%, at least 20%, at least 30%, at least 40%, at least 50%) relative to a control cell that has not been contacted with the RNAi molecule. For example, delivering to a cell (e.g., contacting a cell with) an RNAi molecule (e.g., dsRNA) targeting a plant gene involved in suppression of a systemic or localized protective response pathway may result in a reduction (e.g., by at least 10%, at least 20%, at least 30%, at least 40%, at least 50%) in the amount of RNA transcript and/or protein (e.g., encoded by the plant gene) compared to a cell that is not contacted with an RNAi molecule targeting the plant gene.

In some embodiments, RNAi molecules of the present disclosure specifically inhibit expression of one or more plant genes involved in suppression of a systemic or localized protective response pathway (e.g., SAR pathway) without biologically relevant or biologically significant negative physiological effects (e.g. significant reduction in growth or yield of the plant). In some embodiments, an RNAi molecule specifically inhibits (reduces or blocks) translation of a protein encoded by the plant gene by specifically inhibiting expression of (e.g., degrading) an mRNA (e.g., mRNA encoded by genes comprising a sequence of any one of SEQ ID NOs: 1-86 or 265-274) that encodes the protein. Specific inhibition of a plant gene includes a measurable reduction in the gene expression (e.g., mRNA expression and/or protein expression) or a complete lack of detectable gene expression (e.g., mRNA expression and/or protein expression). In some embodiments, inhibition of a plant gene involves decreased activity of the resultant protein. In some embodiments, inhibition of a plant gene involved in suppression of a systemic or localized protective response pathway further results in increased expression of gene or set of genes involved in triggering or carrying out a systemic or localized protective response pathway, such a gene encoding a protein with antifungal and/or antibacterial properties.

In some embodiments, RNAi molecules of the present disclosure specifically inhibit expression of one or more plant genes involved in suppression of a systemic or localized protective response pathway without biologically relevant or biologically significant off-target effects (no relevant or significant change in the expression of non-target genes). In some embodiments, an RNAi molecule specifically inhibits the expression of a protein encoded by the plant gene by specifically inhibiting the mRNA that encodes the protein (e.g., by specifically inhibiting translation of the mRNA). Specific inhibition of a plant gene involved in suppression of a systemic or localized protective response pathway involves a measurable reduction in the plant gene expression (e.g., mRNA expression and/or protein expression) or a complete lack of detectable gene expression (e.g., mRNA expression and/or protein expression). In some embodiments, inhibition of a plant gene involves decreased activity of the resultant protein.

It should be understood that an “RNAi molecule targeting a plant gene involved in suppression of systemic or localized protective responses to a pathogen” encompasses “RNAi molecules targeting mRNA encoded by the plant gene involved in suppression of systemic or localized protective responses to a pathogen.” A RNAi molecule is considered to target a gene of interest (e.g., a plant gene involved in suppression of systemic or localized protective responses to a pathogen) if the RNAi molecule binds to (e.g., transiently binds to) and inhibits (reduces or blocks) translation of the mRNA, e.g., due to the mRNA being degraded. In some embodiments, an RNAi molecule is a double-stranded RNA molecule. It should also be understood that in some embodiments, the polynucleotide is a double-stranded RNA (e.g., dsRNA) that inhibits expression of a coding region of the gene (e.g., an SAR pathway gene). In other embodiments, the polynucleotide is a DNA sequence that encodes a dsRNA. In yet other embodiments, the polynucleotide is an antisense RNA. It should be understood that the sequences disclosed herein as DNA sequences can be converted from a DNA sequence to an RNA sequence by replacing each thymine with a uracil. And an RNA sequence can be converted to a DNA sequence by replacing each uracil with a thymine.

### Compositions

RNAi molecules targeting a plant gene involved in suppression of a systemic or localized protective response pathway provided herein, in some embodiments, are designed to have complementarity (e.g., partial complementarity or whole complementarity) to mRNA encoded by the plant gene. An example of a DNA sequence encoding a plant gene involved in suppression of a systemic or localized protective response pathway is provided in the sequence of any one of SEQ ID NOs: 1-86 or 265-274 or an ortholog or homolog of any such gene. Examples of RNA molecules targeting a plant gene involved in suppression of a systemic or localized protective response pathway are provided in the sequences of SEQ ID NOs: 87-258 and 275-290. An RNAi molecule has complementarity to an mRNA if it is capable of binding to the mRNA under physiological conditions. In some embodiments, an RNAi molecule is complementary to an mRNA if it base-pairs with at least 50%, at least 60%, at least 70%, at least 80%, at least 85%, at least 90%, at least 95%, or 100% of the mRNA across the length of the RNAi molecule and/or the mRNA.

The term “sequence identity” or “identity,” as used herein in the context of two polynucleotides or polypeptides, refers to the residues in the sequences of the two molecules that are the same when aligned for maximum correspondence over a specified comparison window. Percentage identity is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa. The percent identity of two nucleotide sequences may be determined by comparing two optimally aligned sequences (e.g., nucleic acid sequences or polypeptide sequences) of a molecule over a comparison window, wherein the portion of the sequence in the comparison window may comprise additions or deletions (i.e., gaps) as compared to the reference sequence (which does not comprise additions or deletions) for optimal alignment of the two sequences. Optimal alignment to compare two or more sequences may be performed using local or global alignment through a variety of available computer programs. The algorithm of Smith T. F. and Waterman M. S. (1981), Identification of common molecular subsequences J. Mol. Biol. 147(1):195-, PubMed: 7265238 [SVP]DOI: 10.1016/0022-2836(81)90087-5 is a suitable local alignment strategy and is utilized by tools such as EMBOSS Water (https://www.ebi.ac.uk/Tools/psa/emboss_water/). The algorithm of Needleman S. B. and Wunsch C. D. (1970), A general method applicable to the search for similarities in the amino acid sequence of two proteins, J. Mol. Biol. 48(3):443-53, PubMed: 5420325, DOI: 10.1016/0022-2836(70)90057-4 is a suitable global alignment strategy and is utilized by such tools as EMBOSS Needle (www.ebi.ac.uk/Tools/psa/emboss_needle/). Depending on the sequences to be compared and the relevant parameters, a local or global alignment strategy may be more likely to find an optimal alignment, but both strategies may be utilized to confirm the optimal alignment giving the most accurate percent identity.

In some embodiments, RNAi molecules target two or more genes involved in suppression of a systemic or localized protective response pathway, for example, by using an RNAi molecule with a chimeric design. Such genes may include for example, the genes of SEQ ID NOs: 1-86 or 265-274 or any paralog, ortholog, or homolog thereto. A chimeric trigger may, for example, be a continuous full-length match for the target genes depending on the level of identity. In the case of a low percentage identity, chimeras may be designed, for example, by stitching the sequences from the target genes and therefore not a full-length match for any target.

In some embodiments, an RNAi molecule (e.g., a chimeric dsRNA) comprises a first nucleic acid sequence that targets a first gene for inhibition and a second nucleic acid sequence that targets a second gene for inhibition. The first nucleic acid sequence and/or the second nucleic acid sequence may comprise 18-1000, 18-950, 18-900, 18-850, 18-800, 18-750, 18-700, 18-650, 18-600, 18-500, 18-450, 18-400, 18-350, 18-300, 18-250, 18-200, 18-180, 18-100, 18-50, 19-1000, 19-950, 19-900, 19-850, 19-800, 19-750, 19-700, 19-650, 19-600, 19-500, 19-450, 19-400, 19-350, 19-300, 19-250, 19-200, 19-190, 19-100, 19-50, 20-1000, 20-950, 20-900, 20-850, 20-800, 20-750, 20-700, 20-650, 20-600, 20-500, 20-450, 20-400, 20-350, 20-300, 20-250, 20-200, 20-200, 20-100, 20-50, 21-1000, 21-950, 21-900, 21-850, 21-800, 21-750, 21-700, 21-650, 21-600, 21-500, 21-450, 21-400, 21-350, 21-300, 21-250, 21-210, 21-210, 21-100, 21-50, 22-1000, 22-950, 22-900, 22-850, 22-800, 22-750, 22-700, 22-650, 22-600, 22-500, 22-450, 22-400, 22-350, 22-300, 22-250, 22-220, 22-220, 22-100, 22-50, 23-1000, 23-950, 23-900, 23-850, 23-800, 23-750, 23-700, 23-650, 23-600, 23-500, 23-450, 23-400, 23-350, 23-300, 23-250, 23-230, 23-230, 23-100, 23-50, 24-1000, 24-950, 24-900, 24-850, 24-800, 24-750, 24-700, 24-650, 24-600, 24-500, 24-450, 24-400, 24-350, 24-300, 24-250, 24-240, 24-240, 24-100, 24-50, 25-1000, 25-950, 25-900, 25-850, 25-800, 25-750, 25-700, 25-650, 25-600, 25-500, 25-450, 25-400, 25-350, 25-300, 25-250, 25-250, 25-250, 25-100, or 25-50 nucleotides or nucleotide base pairs. In some embodiments, the first nucleic acid sequence is at least 70%, 80%, 90%, 95%, or 100% complementary to a segment of the first gene. In some embodiments, the second nucleic acid sequence is at least 70%, 80%, 90%, 95%, or 100% complementary to a segment of the second gene.

In some embodiments, the RNAi molecule targeting a plant gene provided herein is designed to have complementarity to a segment of an mRNA encoded by a gene selected from the group consisting of: MPK4, CPR5, MPK4a, RLK3, CPR5, NPR3, RFC3, SNI1, CEV1, COI1, RAP, DMR6-like, ARR9, NPR4, NAC25, BRI1a, BRI1b, TAR2, YUC2a, ETR1, EIN4, ETR2, ACS1, CRY1b, CRY2, ACO4, CTR1, MPK6, RIN4b, RIN4a, RapTOR, TOR, GID1B, GID1C, DHS, GA3OX, HOS15, eIF 5A, CLH2, SPL12l, LsCPR5, LsCPR5, NbEIN5, EIN5, CPR5, WRKY33, WRKY11, HOS15, BZR1, EDR1, EXA1, WRKY40, JAZ7, PW220, MPK6, SPL12I, CEV1, CPR5, COI1, DMR6, DHS, melF-5A, ETR1, EIN4, ETR2, CTR1, MPK4a, RPLK3, BZR1, WRKY40, JAZ, DHS, melF5-A, MPK4a, ETR1, SEIN4, ETR2, CEV1, and CPR5.

In some embodiments, the RNAi molecule targeting a plant gene provided herein is designed to have complementarity to a segment of an mRNA encoded by a gene selected from the group consisting of MPK4, CPR5, MPK4a, RLK3, NPR3, RFC3, SNI1, CEV1, COI1, RAP, DMR6-like, ARR9, NPR4, NAC25, BRI1a, BRI1b, TAR2, YUC2a, ETR1, EIN4, ETR2, ACS1, CRY1b, CRY2, ACO4, CTR1, MPK6, RIN4b, RIN4a, RapTOR, TOR, GID1B, GID1C, DHS, GA3OX, HOS15, eIF_5A, CLH2, SPL12l, EIN5, WRKY33, WRKY11, BZR1, EDR1, EXA1, WRKY40, JAZ7, PW220, DMR6, RPLK3, JAZ, and PAM16. In some embodiments, the RNAi molecule targeting a plant gene provided herein is designed to have complementarity to a segment of an mRNA encoded by a MPK4a, RLK3, RIN4a, CEV1, CPR5, COI1, DHS, ETR1, EIN4, ETR2, PR1, PR3, PR5, or WRKY40 gene. In some embodiments, the RNAi molecule targeting a plant gene provided herein is designed to have complementarity to a segment of an mRNA encoded by a CPR5 gene.

A double-stranded RNA (dsRNA) of the present disclosure, in some embodiments, comprises a first strand that binds to (e.g., is at least partially complementary to or is wholly complementary to) a segment of a messenger RNA (mRNA) encoded by a plant gene described herein (e.g., a gene selected from the group consisting of: MPK4, CPR5, MPK4a, RLK3, NPR3, RFC3, SNI1, CEV1, COI1, RAP, DMR6-like, ARR9, NPR4, NAC25, BRI1a, BRI1b, TAR2, YUC2a, ETR1, EIN4, ETR2, ACS1, CRY1b, CRY2, ACO4, CTR1, MPK6, RIN4b, RIN4a, RapTOR, TOR, GID1B, GID1C, DHS, GA3OX, HOS15, eIF_5A, CLH2, SPL12l, EIN5, WRKY33, WRKY11, BZR1, EDR1, EXA1, WRKY40, JAZ7, PW220, DMR6, RPLK3, JAZ, and PAM16), and a second strand that is complementary to the first strand.

A double-stranded RNA (dsRNA) of the present disclosure, in some embodiments, comprises a first strand that binds to (e.g., is at least partially complementary to or is wholly complementary to) a segment of a messenger RNA (mRNA) encoded by a plant gene described herein (e.g., a gene selected from the group consisting of MPK4, CPR5, MPK4a, RLK3, NPR3, RFC3, SNI1, CEV1, COI1, RAP, DMR6, DMR6-like, ARR9, NPR4, NAC25, BRI1a, BRI1b, TAR2, YUC2a, ETR1, EIN4, ETR2, ETR1/EIN4/ETR2, ACS1, CRY1b, CRY2, ACO4, CTR1, MPK6, RIN4b, RIN4a, RapTOR, TOR, GID1B, GID1C, DHS, GA3OX, HOS15, eIF_5A, CLH2, SPL12l, EIN5, WRKY33, WRKY11, BZR1, EDR1, EXA1, WRKY40, JAZ7, PW220, melF-5A, RPLK3, and JAZ), and a second strand that is complementary to the first strand. dsRNA may comprise RNA strands that are the same length or different lengths. In some embodiments, a dsRNA comprises a first strand (e.g., an antisense strand) that is the same length as a second strand (e.g., a sense strand). In some embodiments, a dsRNA comprises a first strand (e.g., an antisense strand) that is a different length than a second strand (e.g., a sense strand). A first strand may be about 1%, 2%, 3%, 4%, 5%, 10%, 15%, 20%, or more than 20% longer than a second strand. A first strand may be 1-5, 2-5, 2-10, 5-10, 5-15, 10-20, 15-20, or more than 20 nucleotides longer than a second strand.

dsRNA molecules can also be assembled from a single oligonucleotide in a stem-loop structure, wherein self-complementary sense and antisense regions of the RNA molecule are linked by means of a nucleic acid based or non-nucleic acid-based linker(s), as well as circular single-stranded RNA having two or more loop structures and a stem comprising self-complementary sense and antisense strands, wherein the circular RNA can be processed either in vivo or in vitro to generate an active RNAi molecule capable of mediating RNAi. An RNAi molecule may comprise a 3′ overhang at one end of the molecule; the other end may be blunt-ended or have also an overhang (5′ or 3′). When the RNAi molecule comprises an overhang at both ends of the molecule, the length of the overhangs may be the same or different.

A single-stranded RNA of the present disclosure, in some embodiments, comprises a strand that binds to a segment of an mRNA encoded by a mRNA encoded by a plant gene (e.g., a gene selected from the group consisting of: MPK4, CPR5, MPK4a, RLK3, CPR5, NPR3, RFC3, SNI1, CEV1, COI1, RAP, DMR6-like, ARR9, NPR4, NAC25, BRI1a, BRI1b, TAR2, YUC2a, ETR1, EIN4, ETR2, ACS1, CRY1b, CRY2, ACO4, CTR1, MPK6, RIN4b, RIN4a, RapTOR, TOR, GID1B, GID1C, DHS, GA3OX, HOS15, eIF_5A, CLH2, SPL12l, LsCPR5, LsCPR5, NbEIN5, EIN5, CPR5, WRKY33, WRKY11, HOS15, BZR1, EDR1, EXA1, WRKY40, JAZ7, PW220, MPK6, SPL12I, CEV1, CPR5, COI1, DMR6, DHS, melF-5A, ETR1, EIN4, ETR2, CTR1, MPK4a, RPLK3, BZR1, WRKY40, JAZ, DHS, melF5-A, MPK4a, ETR1, SEIN4, ETR2, CEV1, and CPR5).

A single-stranded RNA of the present disclosure, in some embodiments, comprises a strand that binds to a segment of an mRNA encoded by a mRNA encoded by a plant gene (e.g., a gene selected from the group consisting of MPK4, CPR5, MPK4a, RLK3, NPR3, RFC3, SNI1, CEV1, COI1, RAP, DMR6-like, ARR9, NPR4, NAC25, BRI1a, BRI1b, TAR2, YUC2a, ETR1, EIN4, ETR2, ACS1, CRY1b, CRY2, ACO4, CTR1, MPK6, RIN4b, RIN4a, RapTOR, TOR, GID1B, GID1C, DHS, GA3OX, HOS15, eIF_5A, CLH2, SPL12l, EIN5, WRKY33, WRKY11, BZR1, EDR1, EXA1, WRKY40, JAZ7, PW220, DMR6, RPLK3, JAZ, and PAM16).

RNAi molecules as provided herein may vary in length. It should be understood that, in some embodiments, while a long RNA (e.g., dsRNA or ssRNA) molecule is applied to the plant, after entering cells the RNA is cleaved by the Dicer enzyme into shorter RNA fragments having a length of, for example, 15 to 25 nucleotides. Thus, RNAi molecules of the present disclosure may be delivered as 15 to 25 nucleotide fragments, for example, or they may be delivered as longer double-stranded polynucleotides (e.g., at least 100 nucleotides).

Thus, in some embodiments, RNAi molecules targeting a plant gene involved in suppression of a systemic or localized protective response pathway comprise 15-1010 nucleotides (ssRNA) or nucleotide base pairs (dsRNA). For example, an RNAi molecule of the present disclosure may comprise 15-1000, 15-950, 15-900, 15-850, 15-800, 15-750, 15-700, 15-650, 15-600, 15-500, 15-450, 15-400, 15-350, 15-300, 15-250, 15-200, 15-150, 15-100, 15-50, 16-1000, 16-950, 16-900, 16-850, 16-800, 16-750, 16-700, 16-650, 16-600, 16-500, 16-450, 16-400, 16-350, 16-300, 16-250, 16-200, 16-150, 16-100, 16-50, 17-1000, 17-950, 17-900, 17-850, 17-800, 17-750, 17-700, 17-650, 17-600, 17-500, 17-450, 17-400, 17-350, 17-300, 17-250, 17-200, 17-150, 17-100, 17-50, 18-1000, 18-950, 18-900, 18-850, 18-800, 18-750, 18-700, 18-650, 18-600, 18-500, 18-450, 18-400, 18-350, 18-300, 18-250, 18-200, 18-180, 18-100, 18-50, 19-1000, 19-950, 19-900, 19-850, 19-800, 19-750, 19-700, 19-650, 19-600, 19-500, 19-450, 19-400, 19-350, 19-300, 19-250, 19-200, 19-190, 19-100, 19-50, 20-1000, 20-950, 20-900, 20-850, 20-800, 20-750, 20-700, 20-650, 20-600, 20-500, 20-450, 20-400, 20-350, 20-300, 20-250, 20-200, 20-200, 20-100, 20-50, 21-1000, 21-950, 21-900, 21-850, 21-800, 21-750, 21-700, 21-650, 21-600, 21-500, 21-450, 21-400, 21-350, 21-300, 21-250, 21-210, 21-210, 21-100, 21-50, 22-1000, 22-950, 22-900, 22-850, 22-800, 22-750, 22-700, 22-650, 22-600, 22-500, 22-450, 22-400, 22-350, 22-300, 22-250, 22-220, 22-220, 22-100, 22-50, 23-1000, 23-950, 23-900, 23-850, 23-800, 23-750, 23-700, 23-650, 23-600, 23-500, 23-450, 23-400, 23-350, 23-300, 23-250, 23-230, 23-230, 23-100, 23-50, 24-1000, 24-950, 24-900, 24-850, 24-800, 24-750, 24-700, 24-650, 24-600, 24-500, 24-450, 24-400, 24-350, 24-300, 24-250, 24-240, 24-240, 24-100, 24-50, 25-1000, 25-950, 25-900, 25-850, 25-800, 25-750, 25-700, 25-650, 25-600, 25-500, 25-450, 25-400, 25-350, 25-300, 25-250, 25-250, 25-250, 25-100, or 25-50 nucleotides or nucleotide base pairs. In some embodiments, RNAi molecules comprise or consist of at least 15, at least 16, at least 17, at least 18, at least 19, at least 20, at least 21, at least 25, at least 50, at least 200, at least 300, at least 400, at least 500, at least 600, at least 700, at least 800, at least 900, or at least 1000 nucleotides or nucleotide base pairs.

The total length of the RNAi molecules of the present inventions can be greater than or equal to 18 contiguous nucleotides. In some embodiments, an RNAi molecule (e.g., a polynucleotide as described herein) can include nucleotides in addition to the nucleotides that target a plant gene (e.g., a plant gene comprising a sequence selected from the group consisting of SEQ ID NOs: 1-86 or 265-274 or an RNA transcribed therefrom). Similarly, the RNAi molecules of the present disclosure (e.g., a polynucleotide as described herein) may comprise one or more sequences about 75% to about 100% identical to 18 or more contiguous nucleotides of a sequence selected from the group consisting of SEQ ID NOs: 87-258 and 275-290.

In some embodiments, the in addition may comprise additional unrelated sequences. In other words, the total length of the polynucleotide can be greater than the length of the section or segment of the polynucleotide designed to suppress one or more target genes. For example, the polynucleotide can have nucleotides flanking the “active” segment (e.g., an “active” segment could be a sequence complementary to a segment of a target gene or an mRNA transcribed therefrom or could be a sequence selected from SEQ ID NOs: 87-258 and 275-290 that suppresses the target gene, or include “spacer” nucleotides between active segments, or can have additional nucleotides at the 5′ end, or at the 3′ end, or at both the 5′ and 3′ ends. In an embodiment, the RNAi molecule can include additional nucleotides that are not specifically related (having a sequence not complementary or identical to) to the sequences disclosed herein. For example, such RNAi molecules may contain nucleotides that provide stabilizing secondary structure or for convenience in cloning or manufacturing. Or they may be impurities found on the 5′ and/or 3′ end of a strand of RNA. In an embodiment, the RNAi molecule can include additional nucleotides located immediately adjacent to an active segment. In an embodiment, the polynucleotide comprises one such segment, with an additional 5′ G or an additional 3′ C or both, adjacent to the segment. In another embodiment, the polynucleotide is a double-stranded RNA comprising additional nucleotides forming one or more overhangs. In other embodiments, the polynucleotide may comprise one or more active segments recited herein as well as additional segments active against other target genes. Thus, in various embodiments, the nucleotide sequence of the entire RNAi molecule is not 100% identical or complementary to the trigger sequences disclosed herein and is not 100% identical or complementary to a sequence of contiguous nucleotides in the target gene.

In some embodiments, the polynucleotide is complementary to a segment of a plant gene (e.g., a plant gene comprising a nucleic acid sequence having at least 80%, at least 85%, at least 90%, at least 95%, at least 97%, at least 98%, at least 99%, or 100% identity to any one of SEQ ID NOs: 1-86 or 265-274). In some embodiments, the polynucleotide is complementary to a segment of a plant gene (e.g., a plant gene comprising a nucleic acid sequence having at least 80%, at least 85%, at least 90%, at least 95%, at least 97%, at least 98%, at least 99%, or 100% identity to any one of SEQ ID NOs: 1-86 or 265-274), wherein the segment of the plant gene comprises at least 15, at least 20, at least 25, at least 30, at least 40, at least 50, at least 60, at least 70, at least 80, at least 90, or at least 100 contiguous nucleotides of the plant gene.

In some embodiments, an RNAi molecule targeting a plant gene involved in suppression of a systemic or localized protective response pathway comprises or consists of a sequence that is complementary to an mRNA or a segment of an mRNA encoded by the plant gene. In some embodiments, an RNAi molecule targeting a plant gene involved in suppression of a systemic or localized protective response pathway comprises or consists of a sequence that is complementary to an mRNA or a segment of an mRNA encoded by a DNA sequence of any one of SEQ ID NOs: 1-86 or 265-274. In some embodiments, an RNAi molecule targeting a plant gene involved in suppression of a systemic or localized protective response pathway comprises or consists of a sequence that is complementary to an mRNA encoded by a DNA sequence of any one of SEQ ID NOs: 1-86 or 265-274.

In some embodiments, an RNAi molecule targeting a plant gene involved in suppression of a systemic or localized protective response pathway comprises or consists of a sequence that is complementary to a segment of an mRNA encoded by a region or segment of the plant gene DNA. In some embodiments, an RNAi molecule targets an mRNA encoded by a region of a plant gene DNA that may comprise or consist of any sequence encompassed by nucleotides 1 to 500, nucleotides 10 to 500, nucleotides 25 to 500, nucleotides 50 to 500, nucleotides 100 to 500, nucleotides 150 to 500, nucleotides 200 to 500, nucleotides 250 to 500, nucleotides 300 to 500, nucleotides 350 to 500, nucleotides 400 to 500, or nucleotides 450 to 500 of the plant DNA. In some embodiments, an RNAi molecule targets an mRNA encoded by a region of the plant gene DNA that may comprise or consist of any sequence encompassed by nucleotides 200 to 950, nucleotides 250 to 950, nucleotides 300 to 950, nucleotides 350 to 950, nucleotides 400 to 950, nucleotides 450 to 950, nucleotides 500 to 950, nucleotides 550 to 950, nucleotides 200 to 700, nucleotides 250 to 700, nucleotides 300 to 700, nucleotides 350 to 700, nucleotides 400 to 700, nucleotides 450 to 700, nucleotides 500 to 700, nucleotides 550 to 700, nucleotides 600 to 700, or nucleotides 650 to 700 of the plant gene DNA.

It should be understood that the term gene encompasses coding and non-coding nucleic acid. Thus, in some embodiments, a plant gene involved in suppression of a systemic or localized protective response pathway gene encodes an mRNA that comprises a 5′ untranslated region, an open reading frame, and a 3′ untranslated region. Thus, an RNAi molecule herein, in some embodiments, binds to a 5′ untranslated region, an open reading frame, and/or a 3′ untranslated region of an mRNA.

In some embodiments, an RNAi molecule targeting a plant gene involved in suppression of a systemic or localized protective response pathway comprises or consists of an RNA sequence of any one of SEQ ID NOs: 87-258 and 275-290.

In some embodiments, RNAi molecules comprise or consist of a (at least one) contiguous sequence that has 70% to 100% identity (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% identity) to an RNA sequence encoded by a plant gene involved in suppression of a systemic or localized protective response pathway. In some embodiments, the plant gene comprises a DNA sequence of any one of SEQ ID NOs: 1-86 or 265-274. In some embodiments, RNAi molecules comprise or consist of a (at least one) contiguous sequence that has 70% to 100% identity (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% identity) to an RNA sequence encoded by a gene with the DNA sequence of any one of SEQ ID NOs: 1-86 or 265-274 or any homolog, ortholog, or paralog thereto.

In some embodiments, RNAi molecules targeting a plant gene involved in suppression of a systemic or localized protective response pathway comprise or consist of a (at least one) contiguous sequence that is 70% to 100% complementary (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% complementary) to an RNA sequence encoded by the plant gene. In some embodiments, the plant gene comprises a DNA sequence of any one of SEQ ID NOs: 1-86 or 265-274. In some embodiments, RNAi molecules targeting a plant gene involved in suppression of a systemic or localized protective response pathway comprise or consist of a (at least one) contiguous sequence that is 70% to 100% complementary (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% identity) to an RNA sequence encoded by a gene with the DNA sequence of any one of SEQ ID NOS: 1-86 or 265-274 or any homolog, ortholog, or paralog thereto.

In some embodiments, RNAi molecules targeting a plant gene involved in suppression of a systemic or localized protective response pathway comprise or consist of at least 15, at least 16, at least 17, at least 18, at least 19, at least 20, at least 21, at least 25, at least 50, at least 75, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, or at least 1000 nucleotides or nucleotide base pairs having 70% to 100% identity (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% identity) to an RNA sequence or segment of an RNA sequence of any one of SEQ ID NOS: 87-258 and 275-290. In some embodiments, RNAi molecules comprise or consist of at least 15, at least 16, at least 17, at least 18, at least 19, at least 20, at least 21, at least 25, at least 50, at least 75, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, or at least 1000 nucleotides or nucleotide base pairs having 70% to 100% identity (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% identity) to an RNA sequence or segment of an RNA sequence of any one of SEQ ID NO: 87-258 and 275-290. In some embodiments the RNAi molecules comprise one or more sequences identical or complementary to 18 or more contiguous nucleotides of a sequence selected from the group consisting of SEQ ID NO: 87-258 and 275-290. In some embodiments the RNAi molecule comprises one or more sequences at least 90% identical or complementary to 21 or more contiguous nucleotides of a sequence selected from the group consisting of SEQ ID NO: 87-258 and 275-290. In some embodiments the RNAi molecule comprises one or more sequences identical or perfectly complementary to 21 or more contiguous nucleotides of a sequence selected from the group consisting of SEQ ID NO: 87-258 and 275-290.

In some embodiments, RNAi molecules targeting a plant gene involved in suppression of a systemic or localized protective response pathway comprise or consist of at least 15, at least 16, at least 17, at least 18, at least 19, at least 20, at least 21, at least 25, at least 50, at least 75, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, or at least 1000 nucleotides or nucleotide base pairs having 70% to 100% complementary (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% complementary) to an RNA sequence or segment of an RNA sequence of any one of SEQ ID NOS: 87-258 and 275-290. In some embodiments, RNAi molecules targeting a plant gene involved in suppression of a systemic or localized protective response pathway comprise or consist of at least 15, at least 16, at least 17, at least 18, at least 19, at least 20, at least 21, at least 25, at least 50, at least 75, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, or at least 1000 nucleotides or nucleotide base pairs having 70% to 100% complementary (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% complementary) to an RNA sequence or segment of an RNA sequence of any one of SEQ ID NO: 1-86 or 265-274.

In some embodiments, RNAi molecules comprise or consist of 10 to 25, 10 to 24, 10 to 23, 10 to 22, 10 to 21, 10 to 20, 11 to 25, 11 to 24, 11 to 23, 11 to 22, 11 to 21, 11 to 20, 12 to 25, 12 to 24, 12 to 23, 12 to 22, 12 to 21, 12 to 20, 13 to 25, 13 to 24, 13 to 23, 13 to 22, 13 to 21, 13 to 20, 14 to 25, 14 to 24, 14 to 23, 14 to 22, 14 to 21, 14 to 20, 15 to 25, 15 to 24, 15 to 23, 15 to 22, 15 to 21, 15 to 20, 16 to 25, 16 to 24, 16 to 23, 16 to 22, 16 to 21, 16 to 20, 17 to 25, 17 to 24, 17 to 23, 17 to 22, 17 to 21, 17 to 20, 18 to 25, 18 to 24, 18 to 23, 18 to 22, 18 to 21, or 18 to 20 contiguous nucleotides having 70% to 100% identity (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% identity) to an RNA sequence or segment of an RNA sequence of any one of SEQ ID NOS: 87-258 and 275-290. In some embodiments, RNAi molecules comprise or consist of 10 to 25, 10 to 24, 10 to 23, 10 to 22, 10 to 21, 10 to 20, 11 to 25, 11 to 24, 11 to 23, 11 to 22, 11 to 21, 11 to 20, 12 to 25, 12 to 24, 12 to 23, 12 to 22, 12 to 21, 12 to 20, 13 to 25, 13 to 24, 13 to 23, 13 to 22, 13 to 21, 13 to 20, 14 to 25, 14 to 24, 14 to 23, 14 to 22, 14 to 21, 14 to 20, 15 to 25, 15 to 24, 15 to 23, 15 to 22, 15 to 21, 15 to 20, 16 to 25, 16 to 24, 16 to 23, 16 to 22, 16 to 21, 16 to 20, 17 to 25, 17 to 24, 17 to 23, 17 to 22, 17 to 21, 17 to 20, 18 to 25, 18 to 24, 18 to 23, 18 to 22, 18 to 21, or 18 to 20 contiguous nucleotides having 70% to 100% identity (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% identity) to an RNA sequence or segment of an RNA sequence of SEQ ID NO: 87-258 and 275-290.

In some embodiments, RNAi molecules comprise or consist of 10 to 25, 10 to 24, 10 to 23, 10 to 22, 10 to 21, 10 to 20, 11 to 25, 11 to 24, 11 to 23, 11 to 22, 11 to 21, 11 to 20, 12 to 25, 12 to 24, 12 to 23, 12 to 22, 12 to 21, 12 to 20, 13 to 25, 13 to 24, 13 to 23, 13 to 22, 13 to 21, 13 to 20, 14 to 25, 14 to 24, 14 to 23, 14 to 22, 14 to 21, 14 to 20, 15 to 25, 15 to 24, 15 to 23, 15 to 22, 15 to 21, 15 to 20, 16 to 25, 16 to 24, 16 to 23, 16 to 22, 16 to 21, 16 to 20, 17 to 25, 17 to 24, 17 to 23, 17 to 22, 17 to 21, 17 to 20, 18 to 25, 18 to 24, 18 to 23, 18 to 22, 18 to 21, or 18 to 20 contiguous nucleotides having 70% to 100% complementary (e.g., 70% to 100%, 75% to 100%, 80% to 100%, 85% to 100%, 90% to 100%, 95% to 100%, 96% to 100%, 97% to 100%, 98% to 100%, 99% to 100%, 80%, 85%, 90%, 95%, 96%, 97%, 98%, 99%, or 100% complementary) to an RNA sequence or segment of an RNA sequence of any one of SEQ ID NOS: 87-258 and 275-290.

In some embodiments, an RNAi molecule targets any one of the genes set forth in Table 1. In some embodiments, an RNAi molecule targets a gene belonging to one or more of the species provided in Table 1. In some embodiments, an RNAi molecule comprises or consists of any one of the dsRNA sequences provided in Table 1. In some embodiments, an RNAi molecule comprises or consists of any one of the reverse complement sequences provided in Table 2.

Unless otherwise stated, nucleic acid sequences in the text of this specification are given, when read from left to right, in the 5′ to 3′ direction. One of skill in the art would be aware that a given DNA sequence is understood to define a corresponding RNA sequence which is identical to the DNA sequence except for replacement of the thymine (T) nucleotides of the DNA with uracil (U) nucleotides. Thus, providing a specific DNA sequence is understood to define the exact RNA equivalent. A given first polynucleotide sequence, whether DNA or RNA, further defines the sequence of its exact complement (which can be DNA or RNA), a second polynucleotide that hybridizes perfectly to the first polynucleotide by forming Watson-Crick base-pairs. For DNA:DNA duplexes (hybridized strands), base-pairs are adenine:thymine or guanine:cytosine; for DNA:RNA duplexes, base-pairs are adenine:uracil or thymine:adenine or guanine:cytosine. For RNA:RNA duplexes, base-pairs are adenine:uracil or guanine:cytosine Thus, the nucleotide sequence of a blunt-ended double-stranded polynucleotide that is perfectly hybridized (where there is “100% complementarity” between the strands or they are “perfectly complementary”) is unambiguously defined by providing the nucleotide sequence of one strand, whether given as DNA or RNA. By “complementary” to a target gene or a fragment of a target gene or an mRNA or to a second strand of a dsRNA, is meant that a polynucleotide strand (or at least one strand of a double-stranded polynucleotide) is designed to hybridize (generally under physiological conditions such as those found in a plant or fungal cell) to a target gene or to a fragment of a target gene or to the transcript of the target gene or the fragment of a target gene or to the other strand of a dsRNA; one of skill in the art would understand that such hybridization does not necessarily require 100% sequence identity or complementarity. In some embodiments a trigger may be designed such that it is not 100% identical to a sequence of a target gene but remains complementary to a sequence of a target gene or an RNA transcribed therefrom. A first nucleic acid sequence is “operably” connected or “linked” with a second nucleic acid sequence when the first nucleic acid sequence is placed in a functional relationship with the second nucleic acid sequence. For example, a promoter sequence is “operably linked” to a DNA if the promoter provides for transcription or expression of the DNA. Generally, operably linked DNA sequences are contiguous.

The percent identity of two nucleotide sequences may be determined by comparing two optimally aligned sequences (e.g., nucleic acid sequences or polypeptide sequences) of a molecule over a comparison window, wherein the portion of the sequence in the comparison window may comprise additions or deletions (i.e., gaps) as compared to the reference sequence (which does not comprise additions or deletions) for optimal alignment of the two sequences. Optimal alignment to compare two or more sequences may be performed using local or global alignment through a variety of available computer programs. The algorithm of Smith T. F. and Waterman M. S. (1981) Identification of common molecular subsequences. J. Mol. Biol. 147(1):195-7 PubMed: 7265238 DOI: 10.1016/0022-2836(81)90087-5 is a suitable local alignment strategy and is utilized by tools such as EMBOSS Water (www.ebi.ac.uk/Tools/psa/emboss_water/). The algorithm of Needleman S. B. and Wunsch C. D. (1970) A general method applicable to the search for similarities in the amino acid sequence of two proteins. J. Mol. Biol. 48(3):443-53 PubMed: 5420325 DOI: 10.1016/0022-2836(70)90057-4 is a suitable global alignment strategy and is utilized by such tools as EMBOSS Needle (www.ebi.ac.uk/Tools/psa/emboss_needle/). Depending on the sequences to be compared and the relevant parameters, a local or global alignment strategy may be more likely to find an optimal alignment, but both strategies may be utilized to confirm the optimal alignment giving the most accurate percent identity.

The polynucleotides provided herein, such as RNAi molecules targeting a plant gene involved in suppression of a systemic or localized protective response pathway, in some embodiments, are designed to have at least one silencing element complementary (e.g., wholly (100%) or partially (less than 100%, e.g., 90% to 99%) complementary) to a segment of a sequence of mRNA. In some embodiments, polynucleotides comprise at least one silencing element that is essentially identical or essentially complementary to a mRNA of a plant. In some embodiments, the polynucleotides comprise 2 to 5, to 10, 2 to 20, 2 to 20, 2 to 40, or 2 to 50 silencing elements. In some embodiments, the polynucleotides comprise at least 2, at least 3, at least 4, at least 5, at least 6, at least 7, at least 8, at least 9, at least 10, at least 15, at least 20, at least 21, at least 25, at least 30, at least 35, at least 40, at least 45 or at least 50 silencing elements.

RNAi molecules targeting a plant gene involved in suppression of a systemic or localized protective response pathway provided herein may be of any form of RNA, including single-stranded RNA (ssRNA) and double-stranded RNA (dsRNA). Non-limiting examples of single-stranded RNA include mRNA, micro RNA (miRNA) (e.g., artificial miRNA (amiRNA)), small interfering RNA (siRNA), piwi-interacting RNA (piRNA), and antisense RNA. Double-stranded RNA includes wholly double-stranded molecules that do not contain a single-stranded region (e.g., a loop or overhang), as well as partially double-stranded molecules that contain a double-stranded region and a single-stranded region (e.g., a loop or overhang). Further, the RNAi molecules may be single-stranded RNA molecules with secondary structure containing significant double-stranded character, such as, but not limited to, hairpin RNA. Thus, RNAi molecules, in some embodiments, may be short hairpin RNA (shRNA).

In some embodiments, RNAi molecules targeting a plant gene involved in suppression of a systemic or localized protective response pathway comprise dsRNA, ssRNA, siRNA, miRNA (e.g., amirRNA), piRNA, mRNA, or shRNA. In some embodiments, RNAi molecules targeting a plant gene involved in suppression of a systemic or localized protective response pathway comprise more than one form of RNA. For example, the RNAi molecules targeting a plant gene involved in suppression of a systemic or localized protective response pathway may comprise ssRNA and dsRNA. In some embodiments, RNAi molecules targeting a plant gene involved in suppression of a systemic or localized protective response pathway comprise a hybrid with RNA and DNA. In some embodiments, RNAi molecules targeting a plant gene involved in suppression of a systemic or localized protective response pathway comprise amiRNAs processed from a long precursor transcript of nonprotein-coding RNA, that is partially self-complementary to mediate silencing of target mRNAs. amiRNAs are designed, in some embodiments, by replacing the mature 21 nucleotide miRNA sequences within pre-miRNA with 21 nucleotide long fragments derived from the target gene (Frontiers in Plant Science, Sebastian et al., 2017). An amiRNA may have a length of, for example, at least 18 to 500 nucleotides, at least 21 to 500 nucleotides, at least 50 to 500 nucleotides, at least 100 to 500 nucleotides, or at least 200 to 500 nucleotides.

RNAi molecules targeting a plant gene involved in suppression of a systemic or localized protective response pathway may be provided as a mixture of RNAi molecules targeting one or more plant gene involved in suppression of a systemic or localized protective response pathway, for example, a mixture of RNAi molecules targeting different plant genes or homologous gene sequences. Any number of distinct RNAi molecules targeting a plant gene involved in suppression of a systemic or localized protective response pathway may be provided in a mixture of RNAi molecules targeting one or more genes. In some embodiments, the mixture of RNAi molecules targeting plant genes involved in suppression of a systemic or localized protective response pathway comprises at least 2, at least 3, at least 4, at least 5, at least 6, at least 7, at least 8, at least 9, or at least 10 distinct (having different sequences/nucleotide compositions) RNAi molecules targeting different plant genes or homologous gene sequences.

In some embodiment, RNAi molecules targeting a plant gene involved in suppression of a systemic or localized protective response pathway are provided as a mixture of RNAi molecules that are complementary (wholly or partially) to different segments of an mRNA encoded by the same plant gene. Any number of RNAi molecules targeting a plant gene involved in suppression of a systemic or localized protective response pathway that are complementary to different segments of an mRNA (e.g., comprising a sequence encoded by genes comprising any one of SEQ ID NOs: 1-86 or 265-274) may be provided in a mixture of RNAi molecules. In some embodiments, the mixture of RNAi molecules comprises at least 2, at least 3, at least 4, at least 5, at least 6, at least 7, at least 8, at least 9, or at least 10 RNAi molecules targeting a plant gene involved in suppression of a systemic or localized protective response pathway. In some embodiments, the mixture of RNAi molecules comprises 2 to 5, or 2 to 10 RNAi molecules targeting the same plant gene.

In some embodiments, RNAi molecules targeting a plant gene involved in suppression of a systemic or localized protective response pathway provided herein may have one or more mismatches compared with the corresponding sequence of mRNA. A region of complementarity within a RNAi molecule targeting a plant gene involved in suppression of a systemic or localized protective response pathway may have up to 1, up to 2, up to 3, up to 4, etc. mismatches provided that it maintains the ability to form complementary base pairs with mRNA under appropriate hybridization conditions. Alternatively, a region of complementarity on RNAi molecules may have no more than 1, no more than 2, no more than 3, or no more than 4 mismatches provided that the RNAi molecule maintains the ability to form complementary base pairs with mRNA under appropriate hybridization conditions. A region of complementarity within a RNAi molecule may have up to 1, up to 2, up to 3, up to 4, etc. mismatches for every 50, 75, or 100 nucleotides. For example, if an RNAi has a region of complementarity of 100 nucleotides with a target gene then the region of complementarity within the RNAi may have up to 1, up to 2, up to 3, up to 4 mismatches for every 20, 30, 40, or 50 nucleotides (e.g., such that the region of complementarity within the RNAi may have up to 20 mismatches over a 100 nucleotide region of complementarity).

In some embodiments, if there is more than one mismatch in a region of complementarity, they may be positioned consecutively (e.g., 2, 3, 4, or more in a row), or interspersed throughout the region of complementarity provided that the RNAi molecule targeting a plant gene involved in suppression of a systemic or localized protective response pathway maintains the ability to form complementary base pairs with mRNA under appropriate hybridization conditions.

RNAi molecules targeting a plant gene involved in suppression of a systemic or localized protective response pathway may be modified in various ways to improve or control specificity, stability, delivery, bioavailability, degradation, resistance to nuclease degradation, base-pairing properties, RNA distribution, and cellular uptake, and other features relevant to its use. See, e.g., Bramsen et al., Nucleic Acids Res., 2009, 37, 2867-2881; Bramsen and Kjems, Frontiers in Genetics, 3 (2012): 1-22. Accordingly, in some embodiments, RNAi molecules targeting a plant gene involved in suppression of a systemic or localized protective response pathway may include one or more (at least one) suitable modifications. In some embodiments, a modified RNAi molecule targeting a plant gene involved in suppression of a systemic or localized protective response pathway has a modification in its base, sugar (e.g., ribose, deoxyribose), or phosphate group.

RNAi molecules targeting a plant gene involved in suppression of a systemic or localized protective response pathway produced by the methods provided herein may be modified as described herein. In some embodiments, RNAi molecules are produced according to a method described herein and subsequently modified. In some embodiments, RNAi molecules are produced according to a method described herein using a modified starting material. In some embodiments, the modified starting material is a modified nucleobase. In some embodiments, the modified starting material is a modified nucleoside. In some embodiments, the modified starting material is a modified nucleotide.

In some embodiments, modified RNAi molecules targeting a plant gene involved in suppression of a systemic or localized protective response pathway comprise a backbone modification. In some embodiments, backbone modification results in a longer half-life for the RNA due to reduced degradation (e.g., nuclease-mediated degradation). This in turn results in a longer half-life. Examples of suitable backbone modifications include, but are not limited to, phosphorothioate modifications, phosphorodithioate modifications, p-ethoxy modifications, methylphosphonate modifications, methylphosphorothioate modifications, alkyl- and aryl-phosphates (in which the charged phosphonate oxygen is replaced by an alkyl or aryl group), alkylphosphotriesters (in which the charged oxygen moiety is alkylated), peptide nucleic acid (PNA) backbone modifications, and locked nucleic acid (LNA) backbone modifications. These modifications may be used in combination with each other and/or in combination with phosphodiester backbone linkages.

Alternatively or additionally, RNAi molecules targeting a plant gene involved in suppression of a systemic or localized protective response pathway may comprise other modifications, including modifications at the base or sugar moiety. Examples include RNA having sugars that are covalently attached to low molecular weight organic groups other than a hydroxyl group at the 3′ position and other than a phosphate group at the 5′ position (e.g., a 2′-O-alkylated ribose), or RNA having sugars such as arabinose instead of ribose. RNA also embraces substituted purines and pyrimidines such as C-5 propyne modified bases (Wagner et al., Nature Biotechnology 14:840-844, 1996). Other purines and pyrimidines include, but are not limited to, 5-methylcytosine, 2-aminopurine, 2-amino-6-chloropurine, 2,6-diaminopurine, and hypoxanthine. Other such modifications are well known to those of skill in the art.

RNAi molecules that comprise a nucleotide sequence complementary to all or a segment of the target sequence can be designed and prepared using any suitable methods.

### Methods of Use

Aspects of the present disclosure, in some embodiments, provide methods for controlling a pathogen infection comprising delivering to a plant an effective amount of an RNAi molecule targeting a plant gene involved in suppression of a systemic or localized protective response pathway (or a composition comprising an RNAi molecule targeting a plant gene involved in suppression of a systemic or localized protective response pathway). In some embodiments, delivery of the RNAi molecules described herein activates or increases the activity of a systemic or localized protective response pathway of the plant (e.g., as described in Conrath U. Systemic acquired resistance. Plant Signal Behav. 2006 July; 1(4):179-84.; the content of which is incorporated by reference). In some embodiments, a systemic protective response pathway is a systemic acquired resistance pathway.

Further aspects of the present disclosure provide methods of increasing resistance to a pathogens in a plant comprising activating a systemic or localized protective response to a pathogen a systemic or localized protective response pathway of the plant prior to exposure of the plant to the pathogens. In some embodiments, delivery of an RNAi molecule described herein increases the resistance of a plant to pathogens by at least 10%, 20%, 30%, 40%, 50%, 60%, or 70%, relative to a control.

Further aspects of the present disclosure provide methods of increasing the lifespan of a plant, or the average lifespan of a population of plants or increase in the biomass of a population of plants or increase in the yield of a population of plants. In some embodiments, delivery of an RNAi molecule described herein to a plant or a population of plants increases the lifespan of a plant by at least 10%, 20%, 30%, 40%, 50%, 60%, or 70% relative to a control. In some embodiments, delivery of an RNAi molecule described herein to a population of plants increases the biomass of the population of plants by at least 10%, 20%, 30%, 40%, 50%, 60%, or 70%, relative to a control. In some embodiments, delivery of an RNAi molecule described herein to a population of plants increases the yield of the population of plants by at least 10%, 20%, 30%, 40%, 50%, 60%, or 70%, relative to a control.

In some embodiments the RNAi molecule is delivered to the plant prior to infection by a pathogen, for example by delivery to a seed, and functions to suppress expression of a gene involved in suppression of a systemic or localized protective response to the pathogen, thereby activating the systemic or localized protective response prior to infection by the pathogen, the RNAi molecule is delivered before or after infection by the pathogen and resulting in an increase in the plant's ability to control the pathogen. In some embodiments the RNAi molecule functions to suppress expression of a gene involved in suppression of a systemic or localized protective response to the pathogen, thereby resulting in increased expression of a gene involved in activation or increasing activity of the systemic or localized protective response system, resulting in an increase in the plant's ability to control the pathogen.

In this context “control” or “controlling,” includes, but is not limited to, increasing mortality, suppression of growth, suppression of spread from one area of the plant to another, or a decrease in virulence or pathogenicity, or decrease in propagation/reproduction capacity (sporulation) of the pathogen. In some embodiments the pathogen is a fungal pathogens. In some embodiments the fungal pathogen a fungus in the family Fusarium or Phytophthora.

In some embodiments the fungal pathogen is Albugo spp. (white rust) on ornamentals, vegetables (e.g., A. candida) and sunflowers (e.g. A. tragopogo-nis); Alternaria spp. (Alternaria leaf spot) on vegetables, rape (A. brassicola or brassicae), sugar beets (A. tenuis), fruits, rice, soybeans, potatoes (e.g. A. solani or A. alternata), tomatoes (e.g. A. solani or A. alternata) and wheat; Aphanomyces spp. on sugar beets and vegetables; Ascochytaspp. on cereals and vegetables, e.g. A. tritici(anthracnose) on wheat and A. hordei on barley; Bipolaris and Drechslera spp. (teleomorph: Cochliobolus spp.), e.g. Southern leaf blight (D. maydis) or Northern leaf blight (B. zeicola) on corn, e.g. spot blotch (B. sorokiniana) on cereals and e.g. B. oryzae on rice and turfs; Blumeria (formerly Erysiphe) graminis (powdery mildew) on cereals (e.g. on wheat or barley); Botrytis cinerea (teleomorph: Botryotinia fuckeliana: grey mold) on fruits and berries (e.g. strawberries), vegetables (e.g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e.g. C. ulmi (Dutch elm disease) on elms; Cercospora spp. (Cercospora leaf spots) on corn (e.g. Gray leaf spot: C. zeae-maydis), rice, sugar beets (e.g. C. beticola), sugar cane, vegetables, coffee, soybeans (e.g. C. sojina or C. kikuchii) and rice; Cladosporium spp. on tomatoes (e.g. C. fulvum: leaf mold) and cereals, e.g. C. herbarum (black ear) on wheat; Claviceps purpurea (ergot) on cereals; Cochliobolus (anamorph: Helminthasporium of Bipolaris) spp. (leaf spots) on corn (C. carbonum), cereals (e.g. C. sativus, anamorph: B. sorokiniana) and rice (e.g. C. miyabeanus, anamorph: H. oryzae); Colletatrichum (teleomorph: Glomerella) spp. (anthracnose) on cotton (e.g. C. gossypii), corn (e.g. C. graminicola: Anthracnose stalk rot), soft fruits, potatoes (e.g. C. coccodes: black dot), beans (e.g. C. lindemuthianum) and soybeans (e.g. C. truncatum or C. gloeosporioides); Corticium spp., e.g. C. sasakii (sheath blight) on rice; Corynespora cassiicola (leaf spots) on soybeans and ornamentals; Cycloconium spp., e.g. C. oleaginum on olive trees; Cylindrocarpon spp. (e.g. fruit tree canker or young vine decline, teleomorph: Nectria or Neonectria spp.) on fruit trees, vines (e.g. C. liriodendri, teleomorph: Neonectria liriodendri: Black Foot Disease) and ornamentals; Dematophora (teleomorph: Rosellinia) necatrix (root and stem rot) on soybeans; Diaporthe spp., e.g. D. phaseolorum (damping off) on soybeans; Drechslera (syn. Helminthosporium, teleomorph: Pyrenophora) spp. on corn, cereals, such as barley (e.g. D. teres, net blotch) and wheat (e.g. D. tritici−repentis: tan spot), rice and turf; Esca (dieback, apoplexy) on vines, caused by Formitiporia (syn. Phellinus) punctata, F. mediterranea, Phaeomoniella chlamydospora (earlier Phaeoacremonium chlamydosporum), Phaeoacremonium aleophilum and/or Botryosphaeria obtusa; Elsinoe spp. on pome fruits (E. pyri), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e.g. E. pisi), such as cucurbits (e.g., E. cichoracearum), cabbages, rape (e.g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e.g. E. turcicum); Fusarium (teleomorph: Gibberella) spp. (wilt, root or stem rot) on various plants, such as F. graminearum or F. culmorum (root rot, scab or head blight) on cereals (e.g. wheat or barley), F. oxysporum on tomatoes, F. solani (f. sp. glycines now syn. F. virguliforme) and F. tucumaniae and F. brasiliense each causing sudden death syndrome on soybeans, and F. verticillioides on corn; Gaeumannomyces graminis (take-all) on cereals (e.g. wheat or barley) and corn; Gibberella spp. on cereals (e.g. G. zeae) and rice (e.g. G. fujikuroi: Bakanae disease); Glomerella cingulata on vines, pome fruits and other plants and G. gossypii on cotton; Grain staining complex on rice; Guignardia bidwellii (black rot) on vines; Gymnosporangium spp. on rosaceous plants and junipers, e.g. G. sabinae (rust) on pears; Helminthosporium spp. (syn. Drechslera, teleomorph: Cochliobolus) on corn, cereals and rice; Hemileia spp., e.g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e.g. wheat or barley); Microsphaera diffusa (powdery mildew) on soybeans; Monilinia spp., e.g. M. laxa, M. fructicola and M. fructigena (bloom and twig blight, brown rot) on stone fruits and other rosaceous plants; Mycosphaerella spp. on cereals, bananas, soft fruits and ground nuts, such as e.g. M. graminicola (anamorph: Septoria tritici, Septoria blotch) on wheat or M. fijiensis (black Sigatoka disease) on bananas; Peronospora spp. (downy mildew) on cabbage (e.g. P. brassicae), rape (e.g. P. parasitica), onions (e.g. P. destructor), tobacco (P. tabacina) and soybeans (e.g. P. manshurica); Phakopsora pachyrhizi and P. meibomiae (soybean rust) on soybeans; Phialophora spp. e.g. on vines (e.g. P. tracheiphila and P. tetraspora) and soybeans (e.g. P. gregata: stem rot); Phoma lingam (root and stem rot) on rape and cabbage and P. betae (root rot, leaf spot and damping-off) on sugar beets; Phomopsis spp. on sunflowers, vines (e.g. P. viticola: can and leaf spot) and soybeans (e.g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e.g. P. capsici), soybeans (e.g. P. megasperma, syn. P. sojae), potatoes and tomatoes (e.g. P. infestans: late blight) and broad-leaved trees (e.g. P. ramorum: sudden oak death); Plasmodiophora brassicae (club root) on cabbage, rape, radish and other plants; Plasmopara spp., e.g. P. viticola (grapevine downy mildew) on vines and P. halstedii on sunflowers; Podosphaera spp. (powdery mildew) on rosaceous plants, hop, pome and soft fruits, e.g. P. leucotricha on apples; Polymyxa spp., e.g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e.g. wheat or barley; Pseudoperonospora (downy mildew) on various plants, e.g. P. cubensis on cucurbits or P. humili on hop; Pseudopezicula tracheiphila (red fire disease or ‘rotbrenner’, anamorph: Phialophora) on vines; Puccinia spp. (rusts) on various plants, e.g. P. triticina (brown or leaf rust), P. striiformis (stripe or yellow rust), P. hordei (dwarf rust), P. graminis (stem or black rust) or P. recondita (brown or leaf rust) on cereals, such as e.g. wheat, barley or rye, P. kuehnii (orange rust) on sugar cane and P. asparagi on asparagus; Pyrenophora (anamorph: Drechslera) tritici-repentis (tan spot) on wheat or P. teres (net blotch) on barley; Pyricularia spp., e.g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e.g. P. ultimum or P. aphanidermatum); Ramularia spp., e.g. R. collo-cygni (Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e.g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis (scald) on barley, rye and triticale; Sarocladium oryzae and S. attenuatum (sheath rot) on rice; Sclerotinia spp. (stem rot or white mold) on vegetables and field crops, such as rape, sunflowers (e.g. S. sclerotiorum) and soybeans (e.g. S. rolfsii or S. sclerotiorum); Septoria spp. on various plants, e.g. S. glycines (brown spot) on soybeans, S. tritici (Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Setospaeria spp. (leaf blight) on corn (e.g. S. turcicum, syn. Helminthosporium turcicum) and turf; Sphacelotheca spp. (smut) on corn, (e.g. S. reiliana: head smut), sorghum und sugar cane; Sphaerotheca fuliginea (powdery mildew) on cucurbits; Spongospora subterranea (powdery scab) on potatoes and thereby transmitted viral diseases; Stagonospora spp. on cereals, e.g. S. nodorum (Stagonospora blotch, teleomorph: Leptosphaeria [syn. Phaeosphaeria] nodorum) on wheat; Synchytrium endobioticum on potatoes (potato wart disease); Taphrina spp., e.g. T. deformans (leaf curl disease) on peaches and T. pruni (plum pocket) on plums; Thielaviopsis spp. (black root rot) on tobacco, pome fruits, vegetables, soybeans and cotton, e.g. T. basicola (syn. Chalara elegans); Tilletia spp. (common bunt or stinking smut) on cereals, such as e.g. T. tritici (syn. T caries, wheat bunt) and T. cantraversa (dwarf bunt) on wheat; Typhula incarnata (grey snow mold) on barley or wheat; Urocystis spp., e.g. U. occulta (stem smut) on rye; Uromyces spp. (rust) on vegetables, such as beans (e.g. U. appendiculatus, syn. U. phaseoll) and sugar beets (e.g. U. betae); Ustilago spp. (loose smut) on cereals (e.g. U. nuda and U. avaenae), corn (e.g. U. maydis corn smut) and sugar cane; venturia spp. (scab) on apples (e.g. V. inaequalis) and pears; and Verticillium spp. (wilt) on various plants, such as fruits and ornamentals, vines, soft fruits, vegetables and field crops, e.g. V. dahliae on strawberries, rape, potatoes and tomatoes.

In some embodiments, the method of delivery comprises applying to a surface of a plant a composition comprising the RNAi molecule. In some embodiments, a composition comprising an RNAi molecule targeting a plant gene involved in suppression of a systemic or localized protective response pathway is a solid, liquid (including homogeneous mixtures such as a soluble liquid concentrate, and non-homogeneous mixtures such as suspensions, colloids, micelles, and emulsions), powder, suspension, emulsion, spray, encapsulated or micro-encapsulation formulation, in or on microbeads or other carrier particulates, in a film or coating, or on or within a matrix, or as a leaf, seed, root, or stem treatment. In an embodiment, the surface is the leaves, flowers, or fruit of a plant. In some embodiments, a composition comprising an RNAi molecule described herein comprises a carbon quantum dot. A carbon quantum dot is a luminescent nanoparticles comprising carbon that can be endowed with chiral properties. A carbon quantum dot may have low cytotoxicity and facile synthesis. In some embodiments, the carbon quantum dot is as described in Hu et. al., “High efficiency transport of quantum dots into plant roots with the aid of silwet L-77,” Plant Physiology and Biochemistry, 48:703-709 (2010). The carbon quantum dot may be useful for delivery of the RNAi molecule to the plant (e.g., to the seed or leaves of the plant). Carbon dots can be prepared through any method known in the art, including for example, as described in U.S. 2020/0385748 or Hendrix, B. et al. (2021) “Systemic GFP silencing is associated with high transgene expression in Nicotiana benthamiana”, PLOS ONE. Edited by K. R. Davis, 16(3), p. e0245422.

In some embodiments, the composition further comprises an agent that excludes water. In some embodiments, the composition comprises a lipid nanoparticle. In some embodiments, the composition comprises a polyethylene glycol (PEG) molecule. A PEG molecule may be a PEG 200, PEG 500, PEG 1000, PEG 2500, PEG 5000, or any other reasonable PEG molecule.

In some embodiments the composition comprises formulation components described in WO2022/235895, the contents of which are incorporated herein by reference. Such formulation components may, for example, serve to increase the shelf stability of the nucleic acid for suppression of systemic or localized protective response pathway in a plant.

Non limiting examples include emulsifiable concentrates, concentrate solutions, low concentrate solutions, ultra-low volume concentrate solutions, water-soluble concentrate solutions, water-soluble liquid solutions, smoke, fog, invert emulsions, flowables, aerosols, homogenous and non-homogenous mixtures, suspensions (water and oil-based), dust, powders (wettable or soluble), granules (water-dispersible or dry flowables), pellets, capsules, fumigants, encapsulated or micro-encapsulation formulations, or any combinations thereof.

In some embodiments, a composition comprising an RNAi molecule may be applied as a concentrate, spray (after dilution or concentrate), fog, in furrow, seed treatment, seed coating, drench, drip, or any other forms suited for applying to a furrow. The RNAi molecule described herein may be delivered to any portion of a plant, including, but are not limited to, leaf, stem, flower, fruit, shoot, root, seed, tuber, anther, stamen, and/or pollen. In some embodiments, RNAi is delivered mechanically, through high pressure spray or sandblasting.

In some embodiments, the RNAi molecule is delivered to seeds using a seed soaking technique. A seed soaking technique may be as described in WO2013175480; U.S. Pat. Nos. 10,240,161; 10,240,162; 10,934,555; or U.S. Pat. No. 10,683,505; the contents of each of which are incorporated herein by reference. In some embodiments, a seed soaking technique is as described in Halmer, P., Methods to improve seed performance in the field, Handbook of Seed Physiology: Applications to Agriculture; The Haworth Press, Inc., New York (2004), Chapter 5; pp 125-166; the content of which is incorporated herein by reference. In some embodiments, the seek soaking is through the method described in the Examples herein.

A exemplary seed soaking method involves treatment of healthy and uniform seeds with a test formulation diluted with water (e.g., to a working concentration of 0.2 mg/ml of dsRNA at about 30 seeds per 8 ml of diluted formulation). Seeds are then incubated for a period of time (e.g., about 12 to about 24 hours) in the absence of light (and optionally with slow stirring or shaking). After treatment, seeds may be placed in a dish and air-dried (e.g., for 20 to 30 min) before being placed in soil.

In some embodiments the RNAi molecule is delivered to the plant by spraying onto an aboveground plant parts, including, for example, leaves, stems, fruit, vegetable, or flowers. Formulations suitable for spraying known in the art may be used. These include, for example, formulations and methods described in, U.S. 2020/0385748 or Hendrix, B. et al. (2021) ‘Systemic GFP silencing is associated with high transgene expression in Nicotiana benthamiana’, PLOS ONE. Edited by K. R. Davis, 16(3), p. e0245422. Available at: https://doi.org/10.1371/journal.pone.0245422.

In some embodiments, a composition comprises an RNAi molecules and at least one additive selected from adjuvants, attractants, sterilizing agents, growth-regulating substances, carriers or diluents, stabilizers, and/or pesticidal agent(s) (e.g., insecticides, fungicides, and/or herbicides). Formulations components may also be used (e.g. adjuvants, such as antifoaming agents, buffers, compatibility agents, drift control additives, emulsifiers, extenders, invert emulsifiers, plant penetrants, safeners, spreaders, stickers, surfactants, thickeners, and wetting agents).

A composition, in some embodiments, include a mixture of an RNAi molecule targeting a plant gene involved in suppression of a systemic or localized protective response pathway and at least one of a variety of fertilizers, agricultural chemicals, insecticides, miticides, fungicides, pesticidal agents and/or biopesticidal (e.g., microbial, plant-incorporated-protectant (PIP), and/or biochemical) agents, such as Spiromesifen, Spirodiclofen, Spirotetramat, Pyridaben, Tebufenpyrad, Tolfenpyrad, Fenpyroximate, Flufenerim, Pyrimidifen, Fenazaquin, Rotenone, Cyenopyrafen, Hydramethylnon, Acequinocyl, Fluacrypyrim, Aluminium phosphide, Calcium phosphide, Phosphine, Zinc phosphide, Cyanide, Diafenthiuron, Azocyclotin, Cyhexatin, Fenbutatin oxide, Propargite, Tetradifon, Bensultap, Thiocyclam, Thiosultap-sodium, Flonicamid, Etoxazole, Clofentezine, Diflovidazin, Hexythiazox, Chlorfluazuron, Bistrifluron, Diflubenzuron, Flucycloxuron, Flufenoxuron, Hexaflumuron, Lufenuron, Novaluron, Noviflumuron, Teflubenzuron, Triflumuron, Buprofezin, Cyromazine, Hydroprene, Kinoprene, Methoprene, Fenoxycarb, Pyriproxyfen, Pymetrozine, Pyrifluquinazon, Chlorfenapyr, Tralopyril, methyl bromide and/or other alkyl halides, Chloropicrin, Sulfuryl fluoride, Benclothiaz, Chinomethionat, Cryolite, Methylneodecanamide, Benzoximate, Cymiazole, Fluensulfone, Azadirachtin, Bifenazate, Amidoflumet, Dicofol, Plifenate, Cyflumetofen, Pyridalyl, Beauveria bassiana GHA, Sulfoxaflor, Spinetoram, Spinosad, Spinosad, Emamectin benzoate, Lepimectin, Milbemectin, Abamectin, Methoxyfenozide, Chromafenozide, Halofenozide, Tebufenozide, Amitraz, Chlorantraniliprole, Cyantraniliprole, Flubendiamide, alpha-endosulfan, Chlordane, Endosulfan, Fipronil, Acetoprole, Ethiprole, Pyrafluprole, Pyriprole, Indoxacarb, Metaflumizone, Acrinathrin, Allethrin, Allethrin-cis-trans, Allethrin-trans, beta-Cyfluthrin, beta-Cypermethrin, Bifenthrin, Bioallethrin, Bioallethrin S-cyclopentenyl, Bioresmethrin, Cycloprothrin, Cyfluthrin, Cyhalothrin, Cypermethrin, Cyphenothrin [(1R)-trans-isomers], Dimefluthrin, Empenthrin [(EZ)-(1R)-isomers], Esfenvalerate, Etofenprox, Fenpropathrin, Fenvalerate, Flucythrinate, Flumethrin, Gamma-cyhalothrin, lambda-Cyhalothrin, Meperfluthrin, Metofluthrin, Permethrin, Phenothrin [(1R)-trans-isomer], Prallethrin, Profluthrin, Protrifenbute, Resmethrin, Silafluofen, tau-Fluvalinate, Tefluthrin, Tetramethrin, Tetramethrin [(1R)-isomers], Tetramethylfluthrin, theta-Cypermethrin, Tralomethrin, Transfluthrin, zeta-Cypermethrin, alpha-Cypermethrin, Deltamethrin, DDT, Methoxychlor, Thiodicarb, Alanycarb, Aldicarb, Bendiocarb, Benfuracarb, Butoxycarboxim, Carbaryl, Carbofuran, Carbosulfan, Ethiofencarb, Fenobucarb, Formetanate, Furathiocarb, Isoprocarb, Methiocarb, Methomyl, Metolcarb, Oxamyl, Pirimicarb, Propoxur, Thiofanox, Triazamate, Trimethacarb, XMC, Xylylcarb, Chlorpyrifos, Malathion, Acephate, Azamethiphos, Azinphos-ethyl, Azinphos-methyl, Cadusafos, Chlorethoxyfos, Chlorfenvinphos, Chlormephos, Chlorpyrifos-methyl, Coumaphos, Cyanophos, Demeton-S-methyl, Diazinon, Dichlorvos/DDVP, Dicrotophos, Dimethoate, Dimethylvinphos, Disulfoton, EPN, Ethion, Ethoprophos, Famphur, Fenamiphos, Fenitrothion, Fenthion, Fonofos, Fosthiazate, Imicyafos, Isofenphos-methyl, Mecarbam, Methamidophos, Methidathion, Mevinphos, Monocrotophos, Naled, Omethoate, Oxydemeton-methyl, Parathion, Parathion-methyl, Phenthoate, Phorate, Phosalone, Phosmet, Phosphamidon, Phoxim, Pirimiphos-ethyl, Profenofos, Propaphos, Propetamphos, Prothiofos, Pyraclofos, Pyridaphenthion, Quinalphos, Sulfotep, Tebupirimfos, Temephos, Terbufos, Tetrachlorvinphos, Thiometon, Triazophos, Trichlorfon, Vamidothion Imidacloprid, Thiamethoxam, Acetamiprid, Clothianidin, Dinotefuran, Nitenpyram, Nithiozine, Nicotine, Thiacloprid, cyantraniliprole, carbamates, organophosphates, cyclodiene organochlorines, phenylpyrazoles (fiproles), pyrethroids, pyrethins, DDT Methoxychlor, Neonicotinoids, Nicotine, Sulfoximines, Butenolides, Mesoionics, Spinosyns, Avermectins, Milbernycins, Juvenile hormone analogues, Fenoxycarb, Pyriproxyfen, Alkyl halides, Chloropicrin, Fluorides, Borates, Tarter emetic, Methyl isothiocyanate generators, Pyridine azomethine derivatives, Pyropenes, Clofentezine, Diflovidazin, Hexythiazox, Etoxazole, Diafenthiuron, Organotin miticides, Propargite, Tetradifon, Pyrroles, Dinitrophenols, Sulfuramid, Nereistoxin analogues, Benzoylureas, Buprofezin, Cyromazine, Diacylhydrazines, Amitraz, Hydramethylnon, Acequinocyl, Fluacrypyrim, Bifenazate, METI acaricides and insecticides, Rotenone, Oxadiazines, Semicarbazones, Tetronic and Tetramic acid derivatives, Phosphides, Cyanides, Beta-ketonitrile derivatives, Carboxanilides, Diamides, Flonicamid, Meta-diamides Isoxazolines, Granuloviruses (GVs), Nucleopolyhedroviruses (NPVs), GS-omega/kappa HXTX-Hv 1a peptide, Azadirachtin, Benzoximate, Bromopropylate, Chinomethionat, Dicofol, Lime sulfur, Mancozeb, Pyridalyl, Sulfur, Benzimidazoles, Dicarboximides, Pyridines, Pyrimidines, Triazoles, Acylalanines, Pyridine carboxamides, Anilino-pyrimidines, Quinone outside Inhibitors (QoI-fungicides), Phenylpyrroles, Quinolines, Hydroxyanilides, Toluamides, Cyanoacetamide-oximes, Dinitrophenyl crotonates, Phosphonates, Carboxylic Acid Amides (CAA-fungicides), M1 inorganic, M2 inorganic, M3 dithiocarbamates, M4 phthalimides, paraffinic oil, petroleum-based horticultural oils, palmitic oil, steric oil, linoleic oil, oleic oils, canola oil, soybean oil, oregano oil, tagetes oil, balsam fir oil, thyme oil, black pepper oil, mint oil, cedarwood oil, fish oil, jojoba oil, lavadin oil, castor oil, eucalyptus oil, ocimum oil, patchouli oil, citrus oil, artemisia oil, camphor oil, wintergreen oil, methyl eugenol oil, thymol oil, geranium oil, sesame oil, linseed oil, cottonseed oil, lemongrass oil, bergamot oil, mustard oil, orange oil, citronella oil, tea tree oil, neem oil, garlic oil, Bacillus sphaericus, Bacillus thuringiensis (e.g., Bacillus thuringiensis var. aizawai, Bacillus thuringiensis var. israelensis, Bacillus thuringiensis var. kurstaki, Bacillus thuringiensis var. sphaericus, Bacillus thuringiensis var. tenebrionensis) and the insecticidal proteins they produce (e.g., Cry1Ab, Cry1Ac, Cry1Fa, Cry1A.105, Cry2Ab, Vip3A, mCry3A, Cry3Ab, Cry3Bb, Cry34Ab1/Cr35Ab1, and as further exemplified in Crickmore, N., Baum, J., Bravo, A., Lereclus, D., Narva, K., Sampson, K., Schnepf, E., Sun, M. and Zeigler, D. R. “Bacillus thuringiensis toxin nomenclature” (2018)). Paenibacillus popilliae, Serratia entomophila, nuclear polyhedrosis viruses, granulosis viruses, non-occluded baculoviruses, Beauveria spp, Metarhizium, Entomophaga, Zoopthora, Paecilomyces fumosoroseus, Normuraea, Lecanicillium lecanii, Nosema, Thelohania, Vairimorpha, Steinernema spp, Heterorhabditis spp or any combination thereof, which may further comprise an active ingredient selected from the group consisting of azinphos-methyl, acephate, isoxathion, isofenphos, ethion, etrimfos, oxydemeton-methyl, oxydeprofos, quinalphos, chlorpyrifos, chlorpyrifos-methyl, chlorfenvin phos, cyanophos, dioxabenzofos, dichlorvos, disulfoton, dimethylvinphos, dimethoate, sulprofos, diazinon, thiometon, tetrachlorvinphos, temephos, tebupirimfos, terbufos, naled, vamidothion, pyraclofos, pyridafen thion, pirimiphos-methyl, fenitrothion, fenthion, phenthoate, flupyrazophos, prothiofos, propaphos, profenofos, phoxime, phosalone, phosmet, formothion, phorate, malathion, mecarbam, mesulfenfos, methamidophos, methidathion, parathion, methyl parathion, monocrotophos, trichlorphon, EPN, isazophos, isamidofos, cadusafos, diamidaphos, dichlofenthion, thionazin, fenamiphos, fosthiazate, fosthietan, phosphocarb, DSP, ethoprophos, alanycarb, aldicarb, isoprocarb, ethiofen carb, carbaryl, carbosulfan, xylylcarb, thiodicarb, pirimicarb, fenobucarb, furathiocarb, propoxur, ben diocarb, benfuracarb, methomyl, metolcarb, XMC, carbofuran, aldoxycarb, oxamyl, acrin athrin, allethrin, esfenvalerate, empenthrin, cycloprothrin, cyhalothrin, gamma-cyhalothrin, lambda-cyhalothrin, cyfluthrin, beta-cyfluthrin, cypermethrin, alpha-cypermethrin, zeta-cyper-methrin, silafluofen, tetramethrin, tefluthrin, deltamethrin, tralomethrin, bifenthrin, phenothrin, fenvalerate, fenpropathrin, furamethrin, prallethrin, flucythrinate, fluvalinate, flubrocythrinate, permethrin, resmethrin, ethofenprox, cartap, thiocyclam, ben sultap, acetamiprid, imidacloprid, clothianidin, dinotefuran, thiacloprid, thiamethoxam, nitenpyram, chlorfluazuron, difluben zuron, teflubenzuron, triflumuron, novaluron, noviflumuron, bistrifluoron, fluazuron, flucy-cloxuron, flufenoxuron, hexaflumuron, lufenuron, chromafen ozide, tebufenozide, halofen ozide, methoxyfen ozide, diofen olan, cyromazine, pyriproxyfen, buprofezin, methoprene, hydroprene, kinoprene, triazamate, endosulfan, chlorfenson, chlorobenzilate, dicofol, bromopropylate, acetoprole, flpronil, ethiprole, pyrethrin, rotenone, nicotinesulphate, spinosad, finpronil, spirotetramat abamectin, acequinocyl, amidoflumet, amitraz, etoxazole, chinomethionat, clofentezine, fenbutatin oxide, dienochlor, cyhexatin, spirodiclofen, spiromesifen, tetradifon, tebufenpyrad, binapacryl, bifenazate, pyridaben, pyrimidifen, fenazaquin, fenothiocarb, fenpyroximate, fluacrypyrim, fluazinam, flufenzin, hexythiazox, propargite, polynactin complex, milbemectin, lufenuron, mecarbam, methiocarb, mevinphos, halfenprox, azadirachtin, diafenthiuron, indoxacarb, emamectin benzoate, potassium oleate, sodium oleate, chlorfenapyr, tolfenpyrad, pymetrozine, fenoxycarb, hydramethylnon, hydroxy propyl starch, pyridalyl, flufenerim, flubendiamide, flonicamid, metaflumizole, lepimectin, TPIC, albendazole, oxibendazole, oxfendazole, trichlamide, fensulfothion, fenbendazole, levamisole hydrochloride, morantel tartrate, dazomet, metam-sodium, triadimefon, hexaconazole, propiconazole, ipconazole, prochloraz, triflumizole, tebuconazole, epoxiconazole, difenoconazole, flusilazole, triadimenol, cyproconazole, metconazole, fluquinconazole, bitertanol, tetraconazole, triticonazole, flutriafol, penconazole, diniconazole, fenbuconazole, bromuconazole, imibenconazole, simeconazole, myclobutanil, hymexazole, imazalil, furametpyr, thifluzamide, etridiazole, oxpoconazole, oxpoconazole fumarate, pefurazoate, prothioconazole, pyrifenox, fenarimol, nuarimol, bupirimate, mepanipyrim, cyprodinil, pyrimethanil, metalaxyl, mefenoxam, oxadixyl, benalaxyl, thiophanate, thiophanate-methyl, benomyl, carbendazim, fuberidazole, thiabendazole, manzeb, propineb, zineb, metiram, maneb, ziram, thiuram, chlorothalonil, ethaboxam, oxycarboxin, carboxin, flutolanil, silthiofam, mepronil, dimethomorph, fenpropidin, fenpropimorph, spiroxamine, tridemorph, dodemorph, flumorph, azoxystrobin, kresoxim-methyl, metominostrobin, orysastrobin, fluoxastrobin, trifloxystrobin, dimoxystrobin, pyraclostrobin, picoxystrobin, iprodione, procymidone, vinclozolin, chlozolinate, flusulfamide, dazomet, methyl isothiocyanate, chloropicrin, methasulfocarb, hydroxyisoxazole, potassium hydroxyisoxazole, echlomezol, D-D,carbam, basic copper chloride, basic copper sulfate, copper nonylphenolsulfonate, oxine copper, DBEDC, anhydrous copper sulfate, copper sulfate pentahydrate, cupric hydroxide, inorganic sulfur, wettable sulfur, lime sulfur, zinc sulfate, fentin, sodium hydrogen carbonate, potassium hydrogen carbonate, sodium hypochlorite, silver, edifenphos, tolclofos-methyl, fosetyl, iprobenfos, dinocap, pyrazophos, carpropamid, fthalide, tricyclazole, pyroquilon, diclocymet, fenoxanil, kasugamycin, validamycin, polyoxins, blasticiden S, oxytetracycline, mildiomycin, streptomycin, rape seed oil, machine oil, benthiavalicarbisopropyl, iprovalicarb, propamocarb, diethofencarb, fluoroimide, fludioxanil, fenpiclonil, quinoxyfen, oxolinic acid, chlorothalonil, captan, folpet, probenazole, acibenzolar-S-methyl, tiadinil, cyflufenamid, fenhexamid, diflumetorim, metrafenone, picobenzamide, proquinazid, famoxadone, cyazofamid, fenamidone, zoxamide, boscalid, cymoxanil, dithianon, fluazinam, dichlofluanide, triforine, isoprothiolane, ferimzone, diclomezine, tecloftalam, pencycuron, chinomethionat, iminoctadine acetate, iminoctadine albesilate, ambam, polycarbamate, thiadiazine, chloroneb, nickel dimethyldithiocarbamate, guazatine, dodecylguanidine acetate, quintozene, tolylfluanid, anilazine, nitrothalisopropyl, fenitropan, dimethirimol, benthiazole, flumetover, mandipropamide, and penthiopyrad, or any combinations thereof.

In some embodiments, an RNAi molecule targeting a plant gene involved in suppression of a systemic or localized protective response pathway may be applied topically to a plant, or seeds (e.g. via soaking, coating, dusting or spraying), or cells of a plant may be engineered to express the RNAi molecule.

The plant may be any plant that is a common plant crop. In some embodiments, the plant is a Solanaceous plant (e.g., family Solanaceae). Examples of Solanaceous plants include, but are not limited to, potato plants (Solanum tuberosum), buffalo bur plants (Solanum rostratum), eggplant plants (Solanum melongena), tomato plants (Solanum lycopersicum), tobacco plants (Nicotiana tabacum), pepper plants (Capsicum annum) and woody nightshade plants (Solanum dulcamara). In some embodiments, the plant is a vegetable, fruit, or legume. In some embodiments, the plant is soy, spinach, lettuce, cauliflower, pea shoots, or rice.

In some embodiments the plant may be Abelmoschus esculentus, Abelmoschus manihot, Abutilon theophrasti, Acca sellowiana, Acer saccharum, Acmella oleracea, Actinidia arguta, Actinidia chinensis, Actinidia deliciosa, Actinidia kolomikta, Actinidia polygama, Adansonia digitata, Aeschynomene americana, Aframomum melegueta, Agave tequilana, Agropyron cristatum, Allium ampeloprasum, Allium cepa, Allium chinense, Allium fistulosum, Allium ledebourianum, Allium macrostemon, Allium ramosum, Allium sativum, Allium schoenoprasum, Allium scorodoprasum, Allium tuberosum, Alocasia cucullata, Alocasia macrorrhizos, Alpinia galanga, Alpinia globosa, Alpinia malaccensis, Alpinia officinarum, Amaranthus caudatus, Amaranthus cruentus, Amaranthus dubius, Amaranthus hybridus, Amaranthus hypochondriacus, Amaranthus tricolor, Amelanchier alnifolia, Amelanchier canadensis, Ammi majus, Amomum compactum, Amomum verum, Amorphophallus harmandii, Amorphophallus konjac, Amorphophallus paeoniifolius, Amphicarpaea bracteata, Anacardium occidentale, Ananas comosus, Ananas parguazensis, Anethum graveolens, Angelica archangelica, Angelica japonica, Annona cherimola, Annona macroprophyllata, Annona montana, Annona muricata, Annona purpurea, Annona reticulata, Annona scleroderma, Annona squamosa, Anredera baselloides, Anredera cordifolia, Anthoxanthum odoratum, Anthriscus cerefolium, Antidesma bunius, Apios americana, Apium carvi, Apium graveolens, Arachis glabrata, Arachis hypogaea, Arachis villosulicarpa, Aralia cordata, Archidendron bigeminum, Archidendron jiringa, Arctium lappa, Areca catechu, Arenga pinnata, Argania spinosa, Armoracia rusticana, Arracacia xanthorrhiza, Arrhenatherum elatius, Artocarpus altilis, Artocarpus camansi, Artocarpus heterophyllus, Artocarpus integer, Artocarpus lacucha, Artocarpus rigidus, Asclepias syriaca, Asimina triloba, Asparagus officinalis, Atriplex hortensis, Avena abyssinica, Avena sativa, Averrhoa bilimbi, Averrhoa carambola, Baccaurea dulcis, Baccaurea motleyana, Baccaurea racemosa, Baccaurea ramiflora, Bactris gasipaes, Balanites aegyptiaca, Bambusa bambos, Bambusa horsfleldii, Bambusa odashimae, Bambusa vulgaris, Barbarea verna, Barbarea vulgaris, Barringtonia procera, Basella alba, Bassia indica, Bertholletia excelsa, Beta palonga, Beta vulgaris, Bixa orellana, Blighia sapida, Boesenbergia rotunda, Bomarea edulis, Borago ofjicinalis, Bouea macrophylla, Bouteloua curtipendula, Brachiaria deflexa, Brasenia schreberi, Brassica carinata, Brassica juncea, Brassica napus, Brassica nigra, Brassica oleracea, Brassica rapa, Bromelia pinguin, Bromus catharticus, Bromus mango, Bromus marginatus, Bromus secalinus, Brosimum alicastrum, Broussonetia luzonica, Bunchosia armeniaca, Bunchosia costaricensis, Bunium bulbocastanum, Cajanus cajan, Calathea allouia, Calystegia sepium, Camellia japonica, Camellia sinensis, Campanula rapunculus, Campomanesia guaviroba, Campomanesia lineatifolia, Canarium album, Canarium indicum, Canarium ovatum, Canavalia cathartica, Canavalia ensiformis, Canavalia gladiata, Canavalia plagiosperma, Canna indica, Cannabis sativa, Capparis spinosa, Capsicum annuum, Capsicum baccatum, Capsicum chinense, Capsicum pubescens, Cardiocrinum cordatum, Carica papaya, Carica pentagona, Carissa macrocarpa, Carthamus tinctorius, Carum carvi, Carum roxburghianum, Carya cathayensis, Carya illinoinensis, Carya ovata, Caryocar nuciferum, Casimiroa edulis, Castanea crenata, Castanea dentata, Castanea mollissima, Castanea pumila, Castanea sativa, Catha edulis, Celosia argentea, Centrosema pubescens, Ceratonia siliqua, Ceratotheca sesamoides, Cereus hexagonus, Chaenomeles sinensis, Chaerophyllum bulbosum, Chamaedorea tepejilote, Chenopodium album, Chenopodium berlandieri, Chenopodium bonus-henricus, Chenopodium capitatum, Chenopodium foliosum, Chenopodium pallidicaule, Chenopodium quinoa, Chionachne gigantea, Chrysanthemum morifolium, Chrysobalanus icaco, Chrysophyllum africanum, Chrysophyllum cainito, Cicer arietinum, Cichorium endivia, Cichorium intybus, Cinnamomum burmanni, Cinnamomum cassia, Cinnamomum loureiroi, Cinnamomum tamala, Cinnamomum verum, Citrullus lanatus, Citrus aurantiifolia, Citrus aurantium, Citrus hystrix, Citrus jambhiri, Citrus japonica, Citrus limon, Citrus maxima, Citrus medica, Citrus mitis, Citrus paradisi, Citrus reticulata, Citrus sinensis, Clausena lansium, Claytonia perfoliata, Cnidoscolus aconitifolius, Cnidoscolus urens, Cocos nucifera, Coffea arabica, Coffea canephora, Coffea congensis, Coffea eugenioides, Coffea liberica, Cola nitida, Colocasia esculenta, Corchorus olitorius, Corchorus trilocularis, Cordia dodecandra, Cordyline fruticosa, Coriandrum sativum, Corylus avellana, Corylus chinensis, Corylus heterophylla, Corylus maxima, Corylus sieboldiana, Crambe hispanica, Crambe maritima, Crataegus azarolus, Crataegus hupehensis, Crataegus orientalis, Crescentia cujete, Crocus sativus, Crotalaria longirostrata, Croton tiglium, Cryptotaenia japonica, Cucumis melo, Cucumis metuliferus, Cucumis sativus, Cucurbita argyrosperma, Cucurbita ficifolia, Cucurbita maxima, Cucurbita moschata, Cucurbita pepo, Cuminum cyminum, Curcuma angustifolia, Curcuma heyneana, Curcuma longa, Curcuma pierreana, Curcuma zanthorrhiza, Cyamopsis tetragonoloba, Cyclanthera pedata, Cydonia oblonga, Cynara cardunculus, Cyperus cyperoides, Cyperus esculentus, Cyphomandra betacea, Cyrtocarpa procera, Cyrtosperma merkusii, Dacryodes edulis, Daucus carota, Dendrocalamus asper, Dendrocalamus latiflorus, Desmodium discolor, Desmodium intortum, Desmodium uncinatum, Digitaria exilis, Digitaria iburua, Digitaria sanguinalis, Dimocarpus longan, Dioscorea alata, Dioscorea bulbifera, Dioscorea cayennensis, Dioscorea dumetorum, Dioscorea esculenta, Dioscorea flabellifolia, Dioscorea japonica, Dioscorea nummularia, Dioscorea oppositifolia, Dioscorea pentaphylla, Dioscorea piperifolia, Dioscorea quartiniana, Dioscorea trifida, Diospyros discolor, Diospyros ebenum, Diospyros kaki, Diospyros lotus, Diospyros major, Diospyros nigra, Diospyros virginiana, Dipteryx odorata, Dovyalis caffra, Dovyalis hebecarpa, Durio oxleyanus, Durio zibethinus, Dysphania ambrosioides, Echinochloa colona, Echinochloa crus-pavonis, Echinochloa esculenta, Echinochloa frumentacea, Elaeagnus multiflora, Elaeagnus pungens, Elaeagnus umbellata, Elaeis guineensis, Elaeocarpus floribundus, Eleocharis dulcis, Elettaria cardamomum, Eleusine coracana, Elymus canadensis, Elymus caninus, Elymus hispidus, Elymus repens, Elymus smithii, Elymus spicatus, Ensete ventricosum, Enydra fuctuans, Eragrostis tef, Eriobotrya japonica, Eriochloa polystachya, Erioglossum rubiginosum, Eruca vesicaria, Etlingera elatior, Eugenia pyriformis, Eugenia uniflora, Euryale ferox, Euterpe oleracea, Eutrema japonicum, Fagopyrum esculentum, Feronia limonia, Ficus auriculata, Ficus carica, Ficus sycomorus, Flacourtia indica, Flacourtia rukam, Fragaria ananassa, Fragaria chiloensis, Fragaria moschata, Fragaria virginiana, Fragaria viridis, Galega officinalis, Garcinia cochinchinensis, Garcinia dulcis, Garcinia indica, Garcinia madruno, Garcinia mangostana, Garcinia multiflora, Garcinia pedunculata, Garcinia prainiana, Garcinia xanthochymus, Genipa americana, Gigantochloa ligulata, Glebionis coronaria, Glebionis segetum, Gleditsia triacanthos, Glycine max, Glycyrrhiza echinata, Glycyrrhiza glabra, Glycyrrhiza uralensis, Gossypium herbaceum, Gossypium hirsutum, Grewia asiatica, Guizotia abyssinica, Helianthus annuus, Helianthus tuberosus, Hemerocallis fulva, Hibiscus cannabinus, Hibiscus radiatus, Hibiscus sabdariffa, Hodgsonia macrocarpa, Hordeum vulgare, Houttuynia cordata, Hovenia dulcis, Humulus lupulus, Hydrolea zeylanica, Hylocereus undatus, Hyptis suaveolens, Ilex paraguariensis, Illicium verum, Inga edulis, Inga feuilleei, Inga laurina, Inocarpus fagifer, Ipomoea aquatica, Ipomoea batatas, Ipomoea eriocarpa, Ipomoea mammosa, Irvingia gabonensis, Iva annua, Jaltomata procumbens, Jasminum sambac, Jatropha curcas, Jatropha multifida, Juglans ailanthifolia, Juglans hindsii, Juglans honorei, Juglans nigra, Juglans regia, Kaempferia galanga, Kaempferia rotunda, Lablab purpureus, Lactuca indica, Lactuca sativa, Lagenaria siceraria, Lansium parasiticum, Lathyrus cicera, Lathyrus ochrus, Lathyrus sativus, Lathyrus sylvestris, Lathyrus tuberosus, Launaea taraxacifolia, Laurus nobilis, Lecythis zabucajo, Lens culinaris, Lepidium meyenii, Lepidium sativum, Leucaena leucocephala, Levisticum officinale, Lilium auratum, Lilium lancifolium, Lilium leichtlinii, Linum usitatissimum, Lippia adoensis, Litchi chinensis, Litsea calophylla, Lolium perenne, Luffa acutangula, Luffa cylindrica, Lupinus albus, Lupinus angustifolius, Lupinus cosentinii, Lupinus luteus, Lupinus mutabilis, Lupinus perennis, Lupinus tauris, Lycianthes moziniana, Lycium chinense, Macadamia integrifolia, Macrotyloma uniflorum, Malpighia emarginata, Malpighia glabra, Malpighia urens, Malus baccata, Malus domestica, Malus micromalus, Malus prunifolia, Malus sieboldii, Malus spectabilis, Malus turkmenorum, Malva verticillata, Mammea americana, Mangifera caesia, Mangifera foetida, Mangifera indica, Mangifera odorata, Manihot esculenta, Manilkara zapota, Maranta arundinacea, Medicago lupulina, Medicago sativa, Medicago truncatula, Melicoccus bijugatus, Melilotus macrorhizus, Mentha canadensis, Mentha spicata, Mentha suaveolens, Mesua ferrea, Metroxylon sagu, Momordica charantia, Monstera deliciosa, Moringa oleifera, Morus alba, Morus nigra, Mucuna pachylobia, Mucuna pruriens, Muntingia calabura, Murraya koenigii, Musa acuminata, Musa balbisiana, Myrceugenia lanceolata, Myrica rubra, Myristica fragrans, Myrrhis odorata, Nasturtium officinale, Nelumbo nucifera, Nephelium lappaceum, Nephelium mutabile, Neptunia oleracea, Nigella sativa, Nopalea cochenillifera, Nopalea dejecta, Ocimum basilicum, Ocotea quixos, Oenanthe javanica, Olea europaea, Omphalea megacarpa, Opuntia boldinghii, Opuntia crystalenia, Opuntia elatior, Opuntia fcus-indica, Opuntia fusicaulis, Opuntia hyptiacantha, Opuntia leucotricha, Opuntia robusta, Opuntia undulata, Origanum majorana, Origanum vulgare, Oryza glaberrima, Oryza sativa, Osmanthus fragrans, Oxalis deppei, Oxalis tuberosa, Pachyrhizus ahipa, Pachyrhizus erosus, Pachyrhizus tuberosus, Pandanus amaryllifolius, Pandanus brosimos, Pandanus tectorius, Pangium edule, Panicum antidotale, Panicum miliaceum, Panicum virgatum, Parkia biglobosa, Parkia speciosa, Parmentiera aculeata, Parmentiera cereifera, Paspalum dilatatum, Paspalum distichum, Paspalum notatum, Paspalum plicatulum, Paspalum scrobiculatum, Passiflora alata, Passiflora antioquiensis, Passiflora caerulea, Passiflora edulis, Passiflora laurifolia, Passiflora ligularis, Passiflora maliformis, Passiflora mollissima, Passiflora pinnatistipula, Passiflora quadrangularis, Pastinaca sativa, Paullinia cupana, Peltophorum pterocarpum, Pennisetum glaucum, Peperomia pellucida, Pereskia aculeata, Perilla frutescens, Persea americana, Persea schiedeana, Persicaria hydropiper, Petasites japonicus, Petroselinum crispum, Phacelia tanacetifolia, Phaseolus acutifolius, Phaseolus coccineus, Phaseolus dumosus, Phaseolus lunatus, Phaseolus ritensis, Phaseolus vulgaris, Phleum pratense, Phoenix atlantica, Phoenix dactylifera, Phoenix sylvestris, Phragmites australis, Phyllanthus distichus, Phyllanthus emblica, Phyllostachys dulcis, Physalis alkekengi, Physalis ixocarpa, Physalis peruviana, Physalis philadelphica, Physalis pruinosa, Physalis pubescens, Phytolacca acinosa, Pimenta dioica, Pimpinella anisum, Piper auritum, Piper guineense, Piper longum, Piper nigrum, Piper retrofractum, Pistacia vera, Pisum sativum, Pithecellobium dulce, Pithecellobium jiringa, Plantago major, Plectranthus amboinicus, Plectranthus esculentus, Plinia cauliflora, Plukenetia volubilis, Poa compressa, Poa nemoralis, Poa pratensis, Polygala butyracea, Polygonum maximowiczii, Polygonum odoratum, Polymnia sonchifolia, Polyscias fruticosa, Portulaca oleracea, Pouteria caimito, Pouteria campechiana, Pouteria lucuma, Pouteria macrophylla, Pouteria procera, Pouteria sapota, Pouteria viridis, Proboscidea louisianica, Prunus americana, Prunus angustifolia, Prunus armeniaca, Prunus avium, Prunus cerasifera, Prunus cerasus, Prunus domestica, Prunus dulcis, Prunus kansuensis, Prunus mira, Prunus mume, Prunus munsoniana, Prunus nigra, Prunus persica, Prunus pseudocerasus, Prunus salicina, Prunus simonii, Prunus tomentosa, Prunus ussuriensis, Psidium acutangulum, Psidium cattleianum, Psidium friedrichsthalianum, Psidium guajava, Psidium guineense, Psidium sartorianum, Psophocarpus tetragonolobus, Puccinellia nuttalliana, Pueraria montana, Pueraria phaseoloides, Punica granatum, Pyrus bretschneideri, Pyrus communis, Pyrus pyrifolia, Quararibea cordata, Raphanus raphanistrum, Rheum officinale, Rheum rhabarbarum, Rhodomyrtus tomentosa, Ribes aureum, Ribes cynosbati, Ribes longiracemosum, Ribes nigrum, Ribes petraeum, Ribes rubrum, Ribes spicatum, Ribes uva-crispa, Rollinia dolabripetala, Rollinia mucosa, Rolliniopsis discreta, Rorippa indica, Rubus brasiliensis, Rubus chamaemorus, Rubus glaucus, Rubus idaeus, Rubus illecebrosus, Rubus laciniatus, Rubus occidentalis, Rubus phoenicolasius, Rubus rosifolius, Rumex acetosa, Rumex alpinus, Rumex patientia, Rumex scutatus, Saccharum officinarum, Sagittaria sagittifolia, Salacca zalacca, Salsola komarovii, Salsola soda, Salvia hispanica, Salvia officinalis, Sambucus nigra, Sandoricum koetjape, Sanguisorba minor, Sanguisorba officinalis, Sauropus androgynus, Sclerocarya birrea, Secale cereale, Sechium edule, Sechium tacaco, Sedum rupestre, Semecarpus anacardium, Sesamum alatum, Sesamum indicum, Sesamum radiatum, Sesbania grandiflora, Setaria italica, Sida rhombifolia, Sinapis alba, Smallanthus sonchifolius, Solanum aethiopicum, Solanum anomalum, Solanum betaceum, Solanum chaucha, Solanum curtilobum, Solanum duplosinuatum, Solanum goniocalyx, Solanum lycopersicum, Solanum macrocarpon, Solanum melongena, Solanum muricatum, Solanum phureja, Solanum quitoense, Solanum scabrum, Solanum sessiliflorum, Solanum stenotomum, Solanum tuberosum, Solanum uporo, Solanum viride, Sorbus domestica, Sorghum almum, Sorghum bicolor, Sorghum drummondii, Sphenostylis stenocarpa, Spilanthes acmella, Spinacia oleracea, Spondias dulcis, Spondias lakonensis, Spondias mombin, Spondias pinnata, Spondias purpurea, Stachys affinis, Stelechocarpus burahol, Stenocereus griseus, Stenocereus pruinosus, Stenocereus queretaroensis, Stenocereus stellatus, Stevia rebaudiana, Suaeda glauca, Syzygium aromaticum, Syzygium cumini, Syzygium formosum, Syzygium jambos, Syzygium malaccense, Syzygium samarangense, Tacca leontopetaloides, Talinum fruticosum, Talinum paniculatum, Talinum portulacifolium, Talisia esculenta, Tamarindus indica, Tanacetum vulgare, Taraxacum campylodes, Terminalia catappa, Tetragonia tetragonioides, Theobroma bicolor, Theobroma cacao, Theobroma grandiflorum, Theobroma microcarpum, Thymus herba-barona, Thymus vulgaris, Trapa natans, Trichosanthes cucumerina, Trichosanthes japonica, Trichosanthes pilosa, Trifolium alexandrinum, Trifolium ambiguum, Trifolium fragiferum, Trifolium hybridum, Trifolium incarnatum, Trifolium pannonicum, Trifolium pratense, Trifolium repens, Trifolium resupinatum, Trifolium semipilosum, Trifolium subterraneum, Trigonella foenum-graecum, Triphasia trifolia, Tripsacum dactyloides, Triticum aestivum, Triticum boeoticum, Triticum carthlicum, Triticum dicoccon, Triticum durum, Triticum monococcum, Triticum spelta, Triticum sphaerococcum, Triticum timopheevii, Tropaeolum leptophyllum, Tropaeolum tuberosum, Tylosema esculentum, Typha latifolia, Ullucus tuberosus, Urena lobata, Vaccinium ashei, Vaccinium corymbosum, Vaccinium macrocarpon, Vaccinium oxycoccos, Valeriana sambucifolia, Valerianella locusta, Vangueria madagascariensis, Vanilla planifolia, Vanilla pompona, Vanilla tahitensis, Vasconcellea pubescens, Veronica anagallis, Vicia articulata, Vicia benghalensis, Vicia cracca, Vicia ervilia, Vicia faba, Vicia graminea, Vicia hirsuta, Vicia ludoviciana, Vicia monantha, Vicia narbonensis, Vicia pannonica, Vicia sativa, Vicia sepium, Vicia tetrasperma, Vicia unijuga, Vicia villosa, Vigna aconitifolia, Vigna angularis, Vigna cylindrica, Vigna mungo, Vigna radiata, Vigna subterranea, Vigna umbellata, Vigna unguiculata, Vitellaria paradoxa, Vitex doniana, Vitis amurensis, Vitis rotundifolia, Vitis vinifera, Wolffia arrhiza, Xanthium strumarium, Xanthosoma belophyllum, Xanthosoma brasiliense, Xanthosoma caracu, Xanthosoma poeppigii, Xanthosoma sagittifolium, Xanthosoma undipes, Yucca gigantea, Zanthoxylum bungeanum, Zea mays, Zea mexicana, Zingiber mioga, Zingiber officinale, Zizania aquatica, Zizania latifolia, Zizania palustris, or Ziziphus jujuba.

Thus, in some embodiments, the methods comprise delivering to a plant (e.g., a soybean plant) with an RNAi molecule, for example, in an effective amount to suppress infection of the plant thereby controlling a pathogen (e.g., a fungal pathogen). In other embodiments, the methods comprise delivering to a soybean plant with an RNAi molecule described herein, for example, in an effective amount to suppress infection of the plant by pathogen or suppress spread of a pathogen from one part of a plant to another, or suppress spread of a pathogen from one part of a plant to another, or suppress spread of the pathogen from one cell or one cell type to another or suppress spread of the pathogen from one tissue or tissue type to another. In yet other embodiments, the methods comprise delivering to a lettuce plant with an RNAi molecule described herein, for example, in an effective amount to suppress infection of the plant by a pathogen or suppress spread of a pathogen from one part of a plant to another, or suppress spread of the pathogen from one cell or one cell type to another or suppress spread of the pathogen from one tissue or tissue type to another, or suppress spread of a pathogen from one part of a plant to another, or suppress spread of the pathogen from one cell or one cell type to another or suppress spread of the pathogen from one tissue or tissue type to another. In still other embodiments, the methods comprise delivering to a spinach plant with an RNAi molecule described herein, for example, in an effective amount to suppress infection of the plant by a pathogen or suppress spread of a pathogen from one part of a plant to another, or suppress spread of the pathogen from one cell or one cell type to another or suppress spread of the pathogen from one tissue or tissue type to another, or suppress spread of a pathogen from one part of a plant to another, or suppress spread of the pathogen from one cell or one cell type to another or suppress spread of the pathogen from one tissue or tissue type to another.

Delivering to a plant (e.g., a part of a plant) an RNAi molecule may include, for example, applying (e.g., soaking, coating, or dusting) the RNAi molecule or a composition comprising the RNAi molecule topically to any portion of a plant (e.g., roots, tubers, stem, branches, leaves, flower, etc), or ground (e.g., soil, dirt, grass, etc.). A delivering step may also include genetically engineering cells of a plant to express the RNAi molecule.

An effective amount is the amount of an RNAi molecule described herein required to confer a beneficial effect on infection by a pathogen, either alone or in combination with one or more other additives. Beneficial effects include, but are not limited to, increasing mortality, suppression of growth, suppression of spread from one area of the plant to another, or a decrease in spread from one area of the plant to another in virulence or pathogenicity, or decrease in propagation/reproduction capacity (sporulation) of the pathogen. Beneficial effects may be demonstrated by a decrease in affected part surface area or plant part weight, plant part mass or plant part volume affected by the pathogen (optionally wherein the plant part is leaf) by at least 10%, at least 20%, at least 30%, at least 40%, at least 50%, at least 60%, at least 70%, at least 80%, or at least 90%, relative to a control. The plant part for measuring beneficial effects may be, for example, the roots, shoots, fruit, flower, leaf, or any salable part of the plant. Beneficial effects may also be demonstrated by an increase in the functionality fo the plant part vs. control (e.g., an increase in the photosynthesis activity of a leaf) or through an increase in quality of the salable part of a plant. In some embodiments, the control is the absence of the RNAi molecule of the present invention. In some embodiments, an effective amount of an RNAi molecule described herein completely eliminates pathogen infection. Beneficial effects also include decrease in expression of genes involved in suppressing a systemic or localized protective response pathway (e.g., systemic acquired resistance pathways).

Effective amounts vary, as recognized by those skilled in the art, depending on the particular plant, the severity of the infection, the duration of the infection, previous exposure to the pathogen and like factors within the knowledge and expertise of a practitioner. These factors are well known to those of ordinary skill in that art and can be addressed with no more than routine experimentation. It is generally preferred that lower effective concentrations be used, that is, the lowest concentration that provides control of a pathogen, to increase efficiency and decrease cost.

An effective amount of an RNAi molecule described herein may also vary depending on the method of delivery.

In some embodiments, an effective amount of an RNAi molecule is expressed as micrograms (pg) of RNAi molecule per centimeter squared (cm²) of a surface of a plant or ground (e.g., soil, dirt, grass, etc.), i.e., pg/cm². Thus, in some embodiments, an effective amount of an RNAi molecule comprises 0.001 μg/cm² to 10 μg/cm². In some embodiments, an effective amount of an RNAi molecule comprises 0.001 μg/cm² to 9 μg/cm², 0.001 μg/cm² to 8 μg/cm², 0.001 μg/cm² to 7 μg/cm², 0.001 μg/cm² to 6 μg/cm², 0.001 μg/cm² to 5 μg/cm², 0.001 μg/cm² to 4 μg/cm², 0.001 μg/cm² to 3 μg/cm², 0.001 μg/cm² to 2 μg/cm², 0.001 μg/cm² to 1 μg/cm², 0.001 μg/cm² to 0.1 μg/cm², or 0.001 μg/cm² to 0.01 μg/cm². In some embodiments, an effective amount of an RNAi molecule comprises 0.01 μg/cm² to 10 μg/cm², 0.1 μg/cm² to 10 μg/cm², 1 μg/cm² to 10 μg/cm², 2 μg/cm² to 10 μg/cm², 3 μg/cm² to 10 μg/cm², 4 μg/cm² to 10 μg/cm², 5 μg/cm² to 10 μg/cm², 6 μg/cm² to 10 μg/cm², 7 μg/cm² to 10 μg/cm², 8 μg/cm² to 10 μg/cm², or 9 μg/cm² to 10 μg/cm².

In some embodiments, an effective amount of an RNAi molecule is expressed as grams (g) of RNAi molecule per acre (ac.) of a surface of a plant or ground (e.g., soil, dirt, grass, etc.), i.e., g/ac. Thus, in some embodiments, an effective amount of an RNAi molecule comprises 0.01 g/ac. to 100 g/ac. In some embodiments, an effective amount of an RNAi molecule comprises 0.01 g/ac. to 90 g/ac., 0.01 g/ac. to 80 g/ac., 0.01 g/ac. to 70 g/ac., 0.01 g/ac. to 60 g/ac., 0.01 g/ac. to 50 g/ac., 0.01 g/ac. to 40 g/ac., 0.01 g/ac. to 30 g/ac., 0.01 g/ac. to 20 g/ac., 0.01 g/ac. to 10 g/ac., 0.01 g/ac. to 1 g/ac., or 0.01 g/ac. to 0.1 g/ac. In some embodiments, an effective amount of an RNAi molecule comprises 0.1 g/ac. to 100 g/ac., 1 g/ac. to 100 g/ac., 10 g/ac. to 100 g/ac., 20 g/ac. to 100 g/ac., 30 g/ac. to 100 g/ac., 40 g/ac. to 100 g/ac., 50 g/ac. to 100 g/ac., 60 g/ac. to 100 g/ac., 70 g/ac. to 100 g/ac., 80 g/ac. to 100 g/ac., or 90 g/ac. to 100 g/ac.

In some embodiments, an effective amount of an RNAi molecule is expressed as grams of RNA per grams of seed. Thus in some embodiments, an effective amount of an RNAi molecule comprises from about 0.001 μg RNA/g seed to about 10 mg RNA/g seed, from about 0.1 mg RNA/g seed to about 1 mg RNA/g seed, from about 0.1 mg RNA/g seed to about 0.5,g RNA/g seed, from about 0.1 mg RNA/g seed to about 0.3,g RNA/g seed, from about 0.15 g RNA/g seed to about 0.25 mg/g seed, from about 0.01 mg RNA/g seed to about 0.03,g RNA/g seed, from about 0.15 mg RNA/g seed to about 0.25 g RNA/g seed, from about 0.001 μg RNA/g seed to about 10 μg RNA/g seed; or from about 0.001 μg dsRNA/g seed to about to about 1 μg dsRNA/g seed; or from about 0.001 μg dsRNA/g seed to about to about 0.1 μg dsRNA g seed; or from about 0.001 μg dsRNA/g seed to about to about 0.1 μg dsRNA g seed; or from about 0.001 μg dsRNA/g seed to about to about 0.01 μg dsRNA g seed. In some embodiments, an effective amount of an RNAi molecule comprises 10 mg dsRNA/g seed, 5 g dsRNA/g seed, 1 g dsRNA/g seed, 0.95 mg dsRNA/g seed, 0.9 mg dsRNA/g seed, 0.85 mg dsRNA/g seed, 0.8 mg dsRNA/g seed, 0.75 mg dsRNA/g seed, 0.7 mg dsRNA/g seed, 0.65 mg dsRNA/g seed, 0.6 mg dsRNA/g seed, 0.65 mg dsRNA/g seed, 0.5 mg dsRNA/g seed, 0.45 mg dsRNA/g seed, 0.4 mg dsRNA/g seed, 0.35 mg dsRNA/g seed, 0.34 mg dsRNA/g seed, 0.33 mg dsRNA/g seed, 0.32 mg dsRNA/g seed, 0.31 mg dsRNA/g seed, 0.3 mg dsRNA/g seed, 0.29 mg dsRNA/g seed, 0.28 mg dsRNA/g seed, 0.27 mg dsRNA/g seed, 0.26 mg dsRNA/g seed, 0.25 mg dsRNA/g seed, 0.24 mg dsRNA/g seed, 0.23 mg dsRNA/g seed, 0.22 mg dsRNA/g seed, 0.21 mg dsRNA/g seed, 0.2 mg dsRNA/g seed, 0.19 mg dsRNA/g seed, 0.18 mg dsRNA/g seed, 0.17 mg dsRNA/g seed, 0.16 mg dsRNA/g seed, 0.15 mg dsRNA/g seed, 0.14 mg dsRNA/g seed, 0.13 mg dsRNA/g seed, 0.12 mg dsRNA/g seed, 0.11 mg dsRNA/g seed, 0.1 mg dsRNA/g seed, 0.09 mg dsRNA/g seed, 0.08 mg dsRNA/g seed, 0.07 mg dsRNA/g seed, 0.06 mg dsRNA/g seed, 0.05 mg dsRNA/g seed, 0.04 mg dsRNA/g seed, 0.039 mg dsRNA/g seed 0.038 mg dsRNA/g seed 0.037 mg dsRNA/g seed 0.036 mg dsRNA/g seed 0.035 mg dsRNA/g seed 0.034 mg dsRNA/g seed 0.033 mg dsRNA/g seed 0.032 mg dsRNA/g seed, 0.031 mg dsRNA/g seed, 0.03 mg dsRNA/g seed, 0.029 mg dsRNA/g seed 0.028 mg dsRNA/g seed 0.027 mg dsRNA/g seed 0.026 mg dsRNA/g seed 0.025 mg dsRNA/g seed 0.024 mg dsRNA/g seed 0.023 mg dsRNA/g seed 0.022 mg dsRNA/g seed, 0.021 mg dsRNA/g seed, 0.02 mg dsRNA/g seed, 0.019 mg dsRNA/g seed, 0.018 mg dsRNA/g seed 0.017 mg dsRNA/g seed 0.016 mg dsRNA/g seed 0.015 mg dsRNA/g seed 0.014 mg dsRNA/g seed 0.013 mg dsRNA/g seed 0.012 mg dsRNA/g seed 0.011 mg dsRNA/g seed 0.01 mg dsRNA/g seed, 10 μg dsRNA/g seed, 5 μg dsRNA/g seed, 1 μg dsRNA/g seed, 0.5 μg dsRNA/g seed, 0.1 μg dsRNA/g seed, 0.05 μg dsRNA/g seed, or 0.01 μg dsRNA/g seed, or 0.005 μg dsRNA/g seed, or 0.001 μg dsRNA/g seed.

In some embodiments, the effectiveness of an RNAi molecule to control pathogens can be determined using the ability of the RNAi molecule to slow the rate of infection by the pathogen or increased survival of the plant.

In some embodiments, the effectiveness of an RNAi molecule can be determined using the ability of the RNAi molecule to decrease percent plant defoliation by a pathogen. Percent plant defoliation refers to the percentage of plant material (e.g., a soybean leaf) that is destroyed or otherwise affected by a pathogen. In some embodiments, an RNAi molecule causes at least a 10%, 20%, 30%, 40%, 50%, 60%, 70%, 80%, 90%, 95%, 98%, or 100% decrease in the percent plant part surface area or plant part mass or plant part volume affected by a pathogen (optionally wherein the plant part is leaf). In some embodiments, an RNAi molecule causes percent plant part surface area or plant part mass or plant part volume affected by a pathogen (optionally wherein the plant part is leaf) to decrease below 40%, 30, 25%, 20%, 15%, 10%, 5%, 3%, or 1%. In some embodiments, percent plant defoliation remains below 40%, 30, 25%, 20%, 15%, 10%, 5%, 3%, or 1% for at least 5, 6, 7, 8, 9, 10, 15, or 20 days following exposure of the plant to the pathogen. In some embodiments, the ability of an RNAi molecule to decrease percent plant part surface area or plant part mass or plant part volume affected (optionally wherein the plant part is leaf) is compared relative to a control (e.g., a control molecule or untreated conditions). In some embodiments, an RNAi molecule causes percent plant part surface area or plant part mass or plant part volume affected by a pathogen (optionally wherein the plant part is leaf) to decrease by 80%, 70%, 60%, 50%, 40%, 30%, 25%, 20%, 15%, 10%, 5%, 3%, or 1% compared to a control. In some embodiments, percent plant part surface area or plant part mass or plant part volume affected (optionally wherein the plant part is leaf) is measured over time (e.g., over the course of a multi-day exposure of the plant to the pathogen). In some embodiments, percent plant part surface area or plant part mass or plant part volume affected (optionally wherein the plant part is leaf) is measured after 3, 4, 5, 6, 7, 8, 9, 10, or more days of exposure.

In some embodiments, an RNAi molecule may be formulated in a solution. In some embodiments, the effective amount of the RNAi molecule in the solution is expressed as nanograms (ng) or micrograms (pg) of RNAi molecule per milliliter (ml) of the solution, i.e., ng/ml. Thus, in some embodiments, a solution comprises an RNAi molecule at a concentration of 10 ng/ml to 100 μg/ml. In some embodiments, a solution comprises an RNAi molecule at a concentration of 0.001 mg to 0.1 mg/ml, 10 ng/ml to 100 μg/ml, 100 ng/ml to 100 μg/ml, 250 ng/ml to 100 μg/ml, 750 ng/ml to 100 μg/ml, 1000 ng/ml to 100 μg/ml, 10 μg/ml to 100 μg/ml, 25 μg/ml to 100 μg/ml, 50 μg/ml to 100 μg/ml, or 75 μg/ml to 100 μg/ml. In some embodiments, a solution comprises an RNAi molecule at a concentration of 10 ng/ml to 100 μg/ml, 10 ng/ml to 75 μg/ml, 10 ng/ml to 50 μg/ml, 10 ng/ml to 25 μg/ml, 10 ng/ml to 10 μg/ml, 10 ng/ml to 1000 ng/ml, 10 ng/ml to 1000 ng/ml, 10 ng/ml to 750 ng/ml, 10 ng/ml to 500 ng/ml, 10 ng/ml to 250 ng/ml, 10 ng/ml to 100 ng/ml, 10 ng/ml to 75 ng/ml, 10 ng/ml to 50 ng/ml, or 10 ng/ml to 25 ng/ml.

A solution, in some embodiments, comprises an RNAi molecule and at least one additional additive (e.g., a fungicide, surfactant or other non-pesticidal agent). In some embodiments, such a mixture comprises an RNAi molecule at a concentration of 0.0001 μg/ml to 10 μg/ml (e.g., that is applied to a surface of a plant and/or ground (e.g., soil, dirt, grass, etc.)). In some embodiments, such a mixture comprises an RNAi molecule at a concentration of 0.001 μg/ml to 10 μg/ml, 0.01 μg/ml to 10 μg/ml, 0.1 μg/ml to 10 μg/ml, 1 μg/ml to 10 μg/ml, 2 μg/ml to 10 μg/ml, 3 μg/ml to 10 μg/ml, 4 μg/ml to 10 μg/ml, 5 μg/ml to 10 μg/ml, 6 μg/ml to 10 μg/ml, 7 μg/ml to 10 μg/ml, 8 μg/ml to 10 μg/ml, or 9 μg/ml to 10 μg/ml. In some embodiments, such a mixture comprises an RNAi molecule at a concentration of 0.0001 μg/ml to 9 μg/ml, 0.0001 μg/ml to 8 μg/ml, 0.0001 μg/ml to 7 μg/ml, 0.0001 μg/ml to 6 μg/ml, 0.0001 μg/ml to 5 μg/ml, 0.0001 μg/ml to 4 μg/ml, 0.0001 μg/ml to 3 μg/ml, 0.0001 μg/ml to 2 μg/ml, 0.0001 μg/ml to 1 μg/ml, 0.0001 μg/ml to 0.1 μg/ml, 0.0001 μg/ml to 0.01 μg/ml, or 0.0001 μg/ml to 0.001 μg/ml.

The step of delivering to any portion of a plant (e.g., roots, tubers, stem, branches, leaves, flower, etc), ground (e.g., soil, dirt, grass, etc.) with an RNAi molecule may include a single application (single contact) or multiple applications (multiple contacts) of the RNAi molecule to the plant (e.g., seed, root, stem, flower, leaf, tuber) or ground (e.g., soil, dirt, grass, etc). Delivery to a portion of a plant may be in the form of a spray (e.g., pressurized/aerosolized spray, pump) solid, (e.g. powder, pellet, bait), or liquid (e.g., homogeneous mixtures such as solutions and non-homogeneous mixtures such as suspensions (water and oil based), colloids, micelles, and emulsions). The period of time of contact may vary. In some embodiments, delivering comprises an exposure of an RNAi molecule with a portion of a plant for a suitable period sufficient for regulation of the target gene thereby increasing mortality, suppression of growth, suppression of spread from one area of the plant to another, or a decreasing virulence or pathogenicity, or decreasing propagation/reproduction capacity (sporulation) of the pathogen.

In some embodiments, delivering comprises applying an RNAi molecule to a portion of the surface of a plant. In some embodiments, applying an RNAi molecule to a portion of a surface comprises spraying, coating, and/or dusting the surface or portion thereof. In some embodiments, applying an RNAi molecule to a portion of a surface comprises ground drenching or applying the RNAi molecule as a granulated or powdered formulation to the soil adjacent to the roots of the plant.

In some embodiments delivering comprises contacting a seed with an RNAi molecule. In some embodiments, contacting a seed with an RNAi molecule can be accomplished using any method known in the art which allows an effective amount of dsRNA to enter the seed. These examples include, but are not limited to, soaking, spraying, priming, or coating the seed with powder, emulsion, suspension, or solution. In some embodiments, a seed coating or a seed treatment composition comprises an RNAi molecule and at least one plant-enhancing agent, including but not limited to active substances intended to positively influence seed germination, plant emergence, plant growth, plant defense, plant development, and/or plant yield.

A RNAi molecule may be applied to any portion of a plant (e.g., roots, tubers, stem, branches, leaves, flower, etc). In some embodiments, the RNAi molecule is contacted with an above-ground portion of a plant (e.g., a leaf) and/or with a below-ground portion of a plant (e.g., a root), which may include at least one in furrow formulation selected from the group consisting of a powder, granule, pellet, capsule, soluble liquid concentrate, spray (after dilution or concentrate), fog, in furrow, seed treatment, seed coating, drench, drip irrigation, or any other forms suited for applying to a furrow. Portions of a plant that may be contacted with the RNAi molecule described herein include, but are not limited to, leaf, stem, flower, fruit, shoot, root, seed, tuber, anther, stamen, or pollen. In some embodiments, RNAi is delivered mechanically, through high pressure spray or sandblasting.

Consequences of inhibition can be confirmed by any appropriate assay to evaluate one or more properties of a pathogen, or by biochemical techniques that evaluate molecules indicative of expression (e.g., RNA, protein). In some embodiments, the extent to which an RNAi molecule provided herein reduces levels of expression of the target gene is evaluated by comparing expression levels (e.g., mRNA or protein levels) to an appropriate control (e.g., a level of expression in a cell or population of cells to which an RNAi molecule has not been delivered or to which a negative control has been delivered). In some embodiments, an appropriate control level of expression may be a predetermined level or value, such that a control level need not be measured every time. The predetermined level or value can take a variety of forms. In some embodiments, a predetermined level or value can be single cut-off value, such as a median or mean.

In some embodiments, delivering an RNAi molecule targeting a plant gene involved in suppression of a systemic or localized protective response pathway as described herein results in a reduction in the level of expression in a cell of the plant. In some embodiments, the reduction in levels of expression of a plant gene involved in suppression of a systemic or localized protective response pathway may be a reduction by at least 10%, at least 20%, at least 30%, at least 40%, at least 50%, at least 60%, at least 70%, at least 80%, at least 90%, at least 95%, at least 98%, at least 99%, or 100% relative to a control level. In some embodiments, the control level is a level of expression in a similar plant cell (or average level among a population of cells) not contacted with the RNAi molecule. In some embodiments, the control level is a level of expression in a similar plant cell (or average level among a population of cells) contacted with an RNAi molecule targeting a gene not expressed by the plant cell, e.g., green fluorescent protein (GFP) or an RNAi molecule that does not target any plant gene. In some embodiments, the reduction in levels of expression of a plant gene involved in suppression of a systemic or localized protective response pathway results on the increased expression of a gene or genes in a systemic or localized protective response pathway.

In some embodiments, the effect of delivering to a cell or plant an RNAi molecule is assessed after a finite period of time. For example, levels of a target gene may be determined in a cell or plant at least 4 hours, 8 hours, 12 hours, 18 hours, 24 hours; or at least one, two, three, four, five, six, seven, or fourteen days after delivering to the cell or plant the RNAi molecule. In another example (e.g. where the RNAi molecule is delivered to a seed) the levels of a target gene expression may be determined in a cell or plant at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, or 90 days following planting of the seed.

In some embodiments, delivery of an RNAi molecule as described herein results an increase in mortality, suppression of growth, suppression of spread from one area of the plant to another, or a decrease in virulence or pathogenicity, or decrease in propagation/reproduction capacity (sporulation) of the pathogen. In some embodiments, the increase mortality, suppression of growth, suppression of spread from one area of the plant to another, or a decrease in virulence or pathogenicity, or decrease in propagation/reproduction capacity (sporulation) of the pathogen be a reduction or increase, as appropriate, by at least 10%, at least 20%, at least 30%, at least 40%, at least 50%, at least 60%, at least 70%, at least 80%, at least 90%, at least 95%, at least 98%, at least 99%, or 100% relative to a control level. In some embodiments, the control level is mortality, growth, or virulence, pathogenicity, or propagation/reproduction capacity (sporulation) of the pathogen in a plant that has not been contacted with the RNAi molecule.

In some embodiments, delivery of an RNAi molecule as described herein results in an increase in mortality or suppression of growth, suppression of spread from one area of the plant to another, or a decrease in virulence or pathogenicity, or decrease in propagation/reproduction capacity (sporulation) of the pathogen. In some embodiments, the increase in level of mortality may be an increase by at least 10%, at least 20%, at least 30%, at least 40%, at least 50%, at least 60%, at least 70%, at least 80%, at least 90%, at least 95%, at least 98%, at least 99%, or 100% relative to a control. In some embodiments, the control is mortality among the pathogen on plants that have not been contacted with the RNAi molecule.

Aspects of the present disclosure provide plants that express an RNAi molecule as described herein. In some embodiments, DNA encoding an RNAi molecule provided herein is provided to a plant (seed or cells of a plant) such that the plant expresses the RNAi molecule. In some embodiments, DNA encoding an RNAi molecule is expressed in a plant by transgenic expression, e.g., by stably integrating DNA encoding an RNAi molecule into a genome of a plant such that the plant expresses the RNAi molecule.

A pathogen may be pest nematodes (e. g., cyst nematodes Heterodera spp. especially soybean cyst nematode Heterodera glycines, root knot nematodes Meloidogyne spp., lance nematodes Hoplolainus spp., stunt nematodes Tyenchorhynchuts spp., spiral nematodes Helicotylenchus spp., lesion nematodes Praylenchus spp., ring nematodes Criconemna spp., and foliar nematodes Aphelenchus spp. or Aphelenchoides spp. Plant pathogens of interest include any pathogen for which the plant has an innate ability to control via a systemic or localized protective responses. These may include fungi (e. g., the fungi that cause powdery mildew, gray mold, rust, leaf spot and blight, damping-off, root rot, crown rot, cotton boll rot, stem canker, twig canker, vascular wilt, smut, or mold, including, but not limited to, Fusarium spp., Phakospora spp., Rhizoctonia spp., Aspergillus spp., Gibberella spp., Pyricularia spp., Ahernaria spp., and Phytophthora spp.), bacteria (c. g., the bacteria that cause leaf spotting, fireblight, crown gall, and bacterial wilt), mollicutes (e. g., the mycoplasmas that cause yellows disease and spiroplasmas such as Spiroplasma kunkelii, which causes corn stunt), and viruses (e. g., the viruses that cause mosaics, vein banding, flecking, spotting, or abnormal growth).

### Methods of Producing RNAi Molecules

RNAi molecules as provided herein may be produced by any suitable method known in the art. Examples of methods for producing an RNAi molecule include, but are not limited to, in vitro transcription (IVT), chemical synthesis, expression in an organism (e.g., a plant), or expression in cell culture (e.g., a plant cell culture), and microbial fermentation.

RNAi molecules may be produced, in some embodiments, according to cell-free production methods described in International Application Publication WO 2017/176963 A1, published Oct. 12, 2017, entitled “Cell-Free Production of Ribonucleic Acid”; U.S. Provisional Application U.S. Ser. No. 62/571,071 filed Oct. 11, 2017, entitled “Methods and Compositions for Nucleoside Triphosphate and Ribonucleic Acid Production”; and International Application Publication WO 2019/075167 A1, published Apr. 18, 2019, entitled “Methods and Compositions for Nucleoside Triphosphate and Ribonucleic Acid Production”; each of which is incorporated herein by reference.

Any suitable DNA encoding RNAi molecules described herein may be used in the methods described herein. A DNA may be a single-stranded DNA (ssDNA) or a double-stranded DNA (dsDNA). In some embodiments, a DNA comprises one or more DNA expression cassette(s) that when transcribed produces a single-stranded RNA (ssRNA) molecule (e.g., that remains single-stranded or folds into an RNA hairpin) or complementary ssRNA molecules that anneal to produce the double-stranded RNA (dsRNA) molecule.

In some embodiments, a DNA comprises a promoter (e.g., an inducible promoter) operably linked to a nucleotide sequence encoding RNA that is complementary to a segment of a plant gene, and optionally a terminator. In other embodiments, a DNA comprises a first promoter (e.g., an inducible promoter) operably linked to a nucleotide sequence encoding RNA that is complementary to a segment of a plant gene, and optionally a terminator, and a second promoter (e.g., an inducible promoter) operably linked to a nucleotide sequence encoding a second RNA that is complementary to the first RNA, and optionally a terminator. In yet other embodiments, a DNA comprises a promoter (e.g., an inducible promoter) operably linked to a nucleotide sequence encoding a first region of an RNA, followed by one or more nucleotides of a loop region, followed by a second region of the RNA, and optionally followed by a terminator, wherein the first region of the RNA is complementary to a segment of a plant gene and the second region is complementary to the first region. In still other embodiments, a DNA comprises a first strand comprising a first promoter (e.g., an inducible promoter) operably linked to a nucleotide sequence encoding a first RNA that is complementary to a segment of a plant gene, and optionally a terminator, and a second strand comprising a second promoter (e.g., an inducible promoter) operably linked to a nucleotide sequence encoding a second RNA that is complementary to the first RNA, and optionally a terminator wherein the first and second promoters are operably linked to the nucleotide sequence encoding a desired targeting RNA and wherein the bidirectional transcription of the nucleotide sequence encoding the desired targeting RNA results in complementary RNA molecules which anneal to form the dsRNA molecule.

A DNA is typically provided on a vector, such as a plasmid, although other template formats may be used (e.g., linear DNA generated by polymerase chain reaction (PCR), chemical synthesis, or other means known in the art). In some embodiments, more than one DNA is used in a reaction mixture. In some embodiments, 2, 3, 4, 5, or more different DNAs are used in a reaction mixture. In some embodiments the DNA is a construct as described in WO2021/113774 which is incorporated herein by reference.

A promoter or terminator may be a naturally-occurring sequence or an engineered (e.g., synthetic) sequence. In some embodiments, an engineered sequence is modified to enhance transcriptional activity. In some embodiments, the promoter is a naturally-occurring sequence. In other embodiments, the promoter is an engineered sequence. In some embodiments, the terminator is a naturally-occurring sequence. In other embodiments, the terminator is an engineered sequence.

In some embodiments, a polynucleotide (e.g. a dsRNA) is produced according the methods described in U.S. Pat. Nos. 10,954,541; and 10,858,385, the entire content of each of which is incorporated herein by reference.

### EXAMPLES

In order that the invention described herein may be more fully understood, the following examples are set forth. The Examples described in this Application are offered to illustrate the methods, compositions, and systems provided herein and are not to be construed in any way as limiting their scope.

### Example 1. Delivery of Double-Stranded RNA Via Carbon Dots

Carbon quantum dots can deliver dsRNA (LD045, GmMPK4a, GmRLK3) that target a plant gene involved in suppression of a systemic or localized protective response pathway to soy plants to confer a visible phenotype in first true leaves at 12 days after planting. Carbon dots were compared to a self-assembling lipid nanoparticle formulant for the ability to deliver dsRNA to the cytoplasm of soy plants by visual assessment of leaf phenotypes associated with high levels of downregulation of the gene targets GS200 and GS201. Certain carbon dots (CQD3) described herein were prepared by mixing 9 mL of carbowax PEG 200 with 3 mL of water on a magnetic stir plate. In a separate vessel, 100 mg branched PEI, 800 Da were weighed into a glass scintillation vial to which 2 mL of water were added. The sample was mixed on a magnetic stir plate and then the PEG solution was poured over top. The sample was allowed to mix until homogeneous. Then the solution was transferred to a larger vessel and microwaved for 3 minutes in the 1350W consumer microwave, the average temperature was 170° C. The resulting pale-yellow solution was allowed to cool to RT before use in formulation. Alternatively, the 3-minute 1350W consumer microwave step can be omitted to form a non-dot version with simple complexation of dsRNA with PEI stabilized with PEG200. Healthy and uniform seeds were hand-picked, washed with water for 2 h-3 h at room temp and dried for 4 h to O/N at 25° C. to 28° C. Seeds were treated with the test formulation (1.5 g/L) diluted with water to a working concentration of 0.2 mg/ml of dsRNA at about 30 seeds per 8 ml of diluted formulation. Seeds were incubated in this example for 12 or 24 hours in the dark with slow shaking conditions. After treatment, seeds were placed in a Petri dish and air-dried for 20 to 30 min and placed in soil. Plants were grown under long day conditions at 25° C. day (15 hrs) and 20° C. night (9 hrs) in a greenhouse at Lat 36.05, Long −78.95. Soil used was Fafard 2 mix composed of 75% Canadian Sphagnum Peat Moss, 20% Perlite, 5% Vermiculite, Dolomite Lime, Long-Lasting Wetting Agent, RESiLIENCE. Plants were fertilized with half strength Hoaglands, of approximately 150 ppm, once weekly on Thursdays and 1× Hoaglands once weekly at approximately 300 ppm on Wednesdays. Phenotypes and germination rates were scored visually at 12 days after planting.

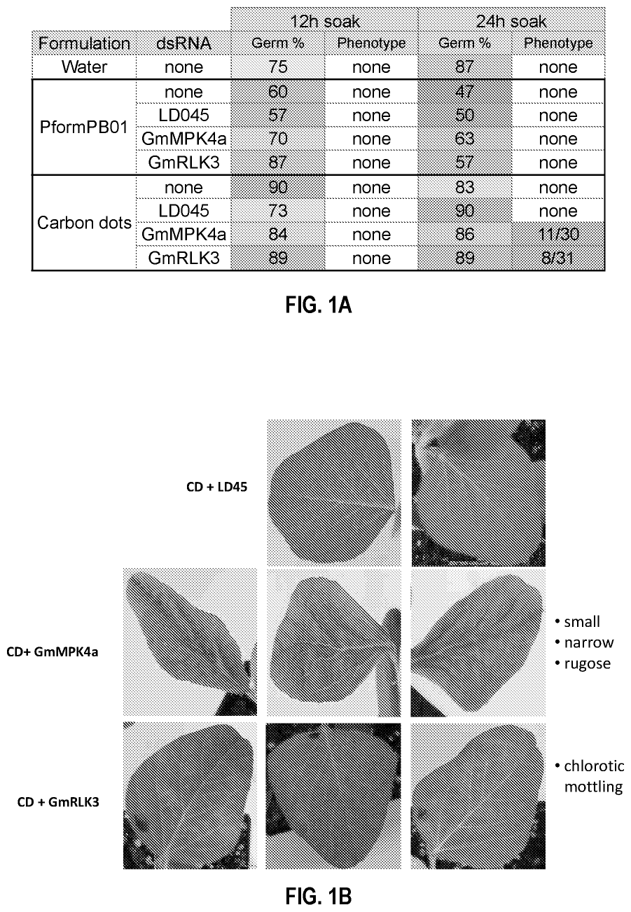

FIG. 1A is a table of outcomes; carbon dots produced a visible leaf phenotype for GmMPK4a and GmRLK3 while water (negative control) and PformPB01 (seed soaking) did not. FIG. 1B are representative images of the leaf phenotypes seen compared to a non-targeting control dsRNA LD045 (does not target an SAR pathway suppressor gene).

### Example 2. DsRNA Molecules Targeting Genes Involved in Suppression of Pathogen Defense Pathways

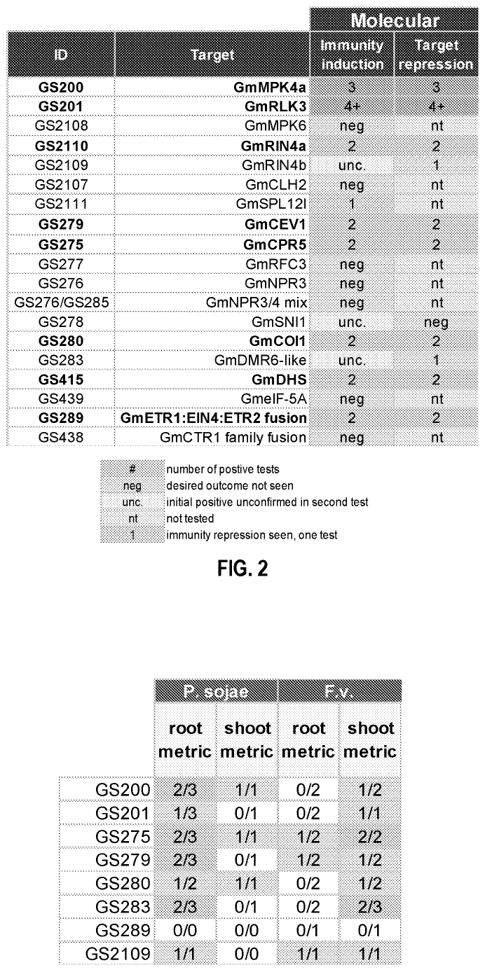

dsRNA molecules targeting systemic plant genes involved in suppression of a systemic or localized protective response pathways can upregulate pathogen defense pathways (e.g., systemic acquired resistance pathway) in soybean plants. This regulation was associated with repression of the intended target gene. Delivery was by seed soaking as described in Example 1 except that the soaking was overnight for approximately 18 hours in formulated dsRNA at 0.2 mg/ml. Leaves were sampled by hole punch of the first true trifoliate leaves to test for gene expression by qRT-PCR between 15 and 37 days after seeds were sown in soil pots and grown in the greenhouse at a range of 21° C. to 25° C. day (15 hours) and 20° C. to 21° C. night (9 hours). Of the 19 targets tested, 8 were confirmed to have this activity (MPK4a, RLK3, RIN4a, CEV1, CPR5, COI1, DHS, and a chimeric dsRNA (targeting ETR1/EIN4/ETR2). Results are shown in FIG. 2.

### Example 3. DsRNA Targeting Genes Involved in Suppression of Pathogen Defense Pathways Protect Plant Seedlings from Fungal Pathogens

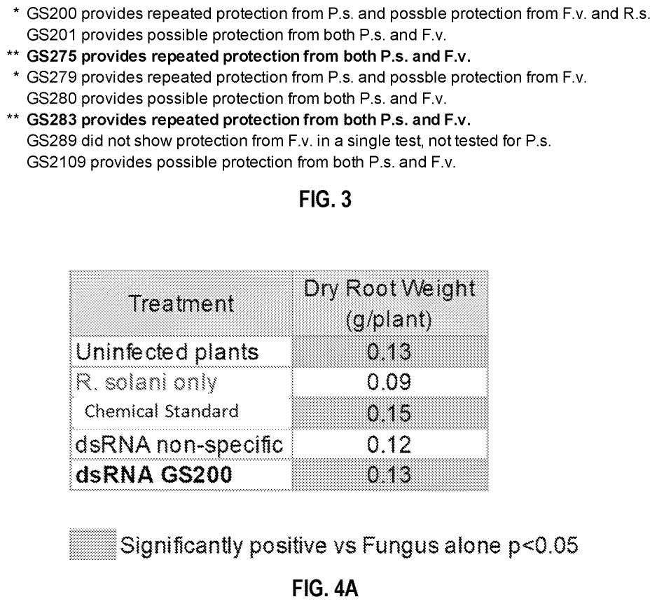

dsRNA molecules targeting putative SAR pathway suppressor genes can protect soybean seedlings from fungal disease (FIG. 3). Delivery was by seed soaking in formulated dsRNA at 0.2 mg/ml as described in Example 2. dsRNA treated seeds were sown in the presence of fungal inoculum using vermiculite substrate instead of soil and watered with 0.5× Hoaglands as needed. (either fungal pathogen belonging to the genus Fusarium (F.v.) or the genus Phytophthora (P.s.)) and compared to mock treated controls. Two root and two shoot metrics were disease scores (determined by visual inspection) and dry tissue weights at 14 days after planting. Statistical evaluation of the 7 replicates per treatment was done using the Tukey HSD test and p<0.05 taken as significant. The fraction in the table indicates how many significantly positive tests were seen (numerator) out of how many viable tests were completed (denominator).

If either of the two metrics, visual score or weight, was positive, it was counted as a positive outcome for that tissue type. Root impacts are thought to be more relevant for P.s. and shoot metrics more relevant for F.v.

### Example 4. DsRNA Targeting Genes Involved in Suppression of Pathogen Defense Pathways Protects Plant Seedlings from Fungal Pathogens

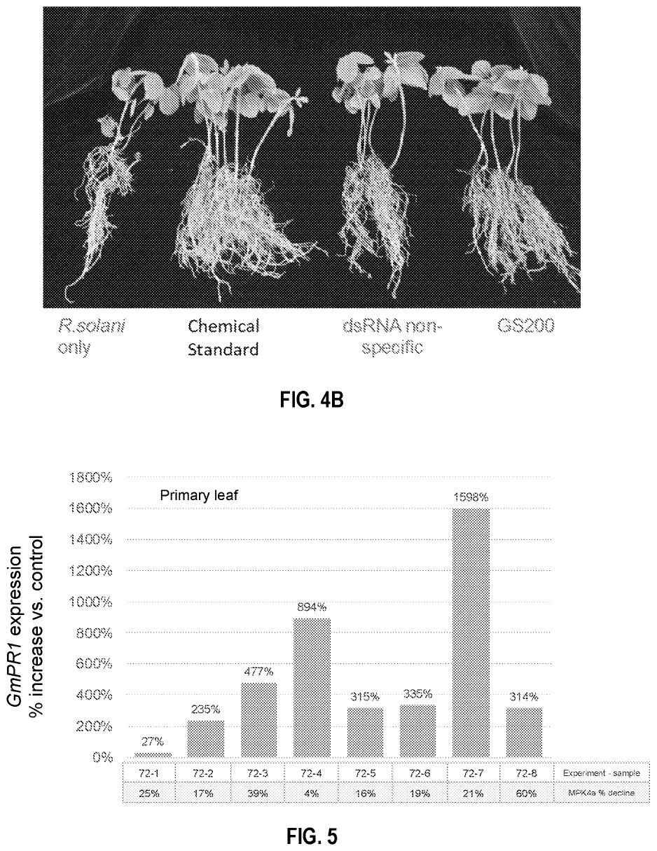

A dsRNA targeting GmMPK4a (GS200) was applied as a seed treatment as described in Example 2 for soybean seedlings and protected the seedlings from Rhizoctonia solani infection (FIGS. 4A-4B). Methods of plant growth and evaluation were the same as in Example 3 except that Rhizoctonia solani fungal inoculum was used. Root weight for R.s infected seedlings treated with GS200 via the seed is equivalent to that of a commercial chemical fungicide. Both commercial treatment and the GS200 treatment result in roots that weigh more than the control R. solani infected seedlings. Non-specific dsRNA control treatment did not result in greater root weights vs. infected control.

### Example 5. DsRNAs Targeting Genes Involved in Suppression of Pathogen Defense Pathways

Further testing was undertaken to examine whether various RNAi molecules with trigger sequences that downregulate expression of response repressor genes correspondingly resulted upregulation of markers of defense response. Markers of defense responses include, for example, PR1, PR3, and PR5. These marker genes are expressed when the plant's defense response pathways are activated. Testing was conducted as described in Example 2 with expression measured for the target response repressor gene and a marker of defense responses.

FIG. 5 shows the increased GmPR1 expression as percentage increase vs. the control for GS200/GmMPK4a. Individual seedlings were labeled 1 to 8. The control was a non-targeting dsRNA and the seed soak method from Example 2 was used with a formulation of CQD3 and the normalizer was GmActin. The primary leaf, sample 72 was day 37 post treatment. Sample 72 used a single control group.

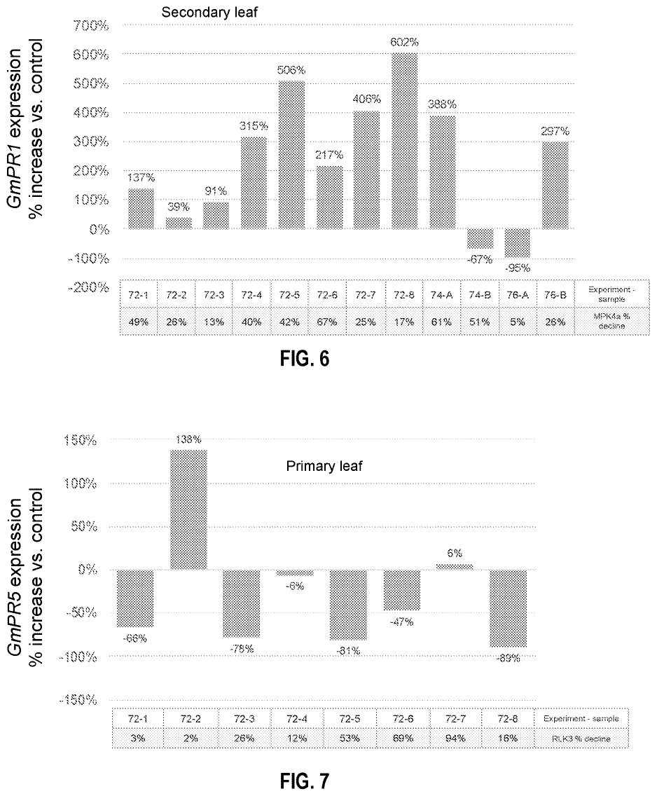

FIG. 6 shows the increased GmPR1 expression as percentage increase vs. the control for GS200/GmMPK4a. Individual seedlings were labeled 1 to 8. The control was LD45 and the seed soak method, was used with the formulation of CQD3 and the normalizer was GmActin. The secondary leaf, sample 72 was day 37 post treatment, sample 74 was day 18 post treatment, and sample 76 was day 23 post treatment. Samples 72 and 74 used a single control group. Sample 76 used an average of two control groups. The seed soak method from Example 2 was used.

FIG. 7 shows the increased GmPR5 expression as percentage increase vs. the control for GS201/GmRLK3. Individual seedlings were labeled 1 to 8. The control was LD45 and the seed soak method from Example 2 was used with the formulation of CQD3 and the normalizer was GmActin. The primary leaf, sample 72 was day 37 post treatment. sample 72 used a single control group.

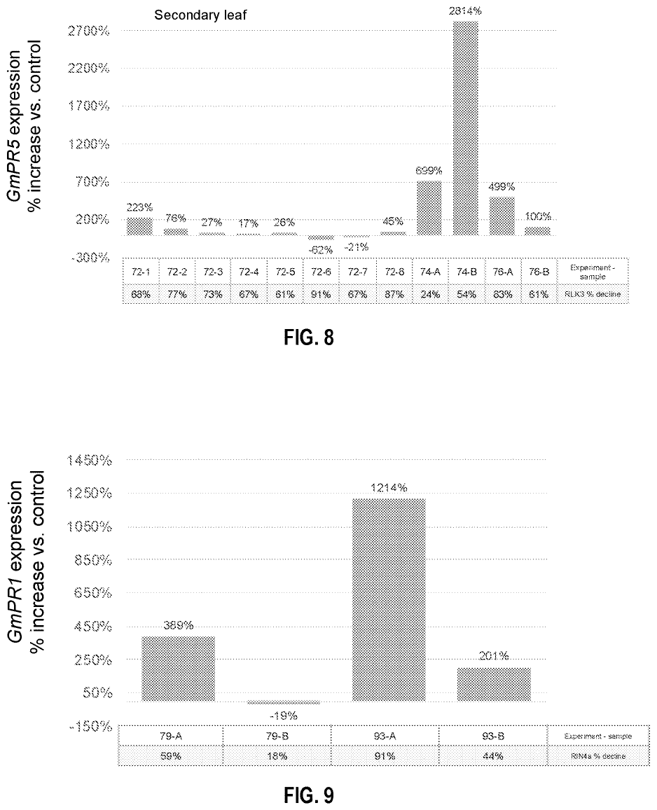

FIG. 8 shows the increased GmPR5 expression as percentage increase vs. the control for GS201/GmRLK3. Individual seedlings were labeled 1 to 8. The control was LD45 and the seed soak method was used with a formulation of CQD3+bPEI800 (Heated) with the normalizer GmActin. The secondary leaf, sample 72 was day 37 post treatment. Sample 74 was day 18 post treatment, and sample 76 was day 23 post treatment. Samples 72 and 74 used a single control group. Sample 76 used an average of two control groups. The seed soak method from Example 2 was used.

FIG. 9 shows the increased GmPR1 expression as percentage increase vs. the control for GS2110/GmRIN4a. The control LD45-average between two independent samples, A and B, was used for the comparison of sample 79 and a single pooled group was used for sample 93. The seed soak method from Example 2 was used with a formulation of CQD3 with the normalizer GmActin. The secondary leaf, sample 79 was day 21 post treatment and sample 93 was day 22 post treatment.

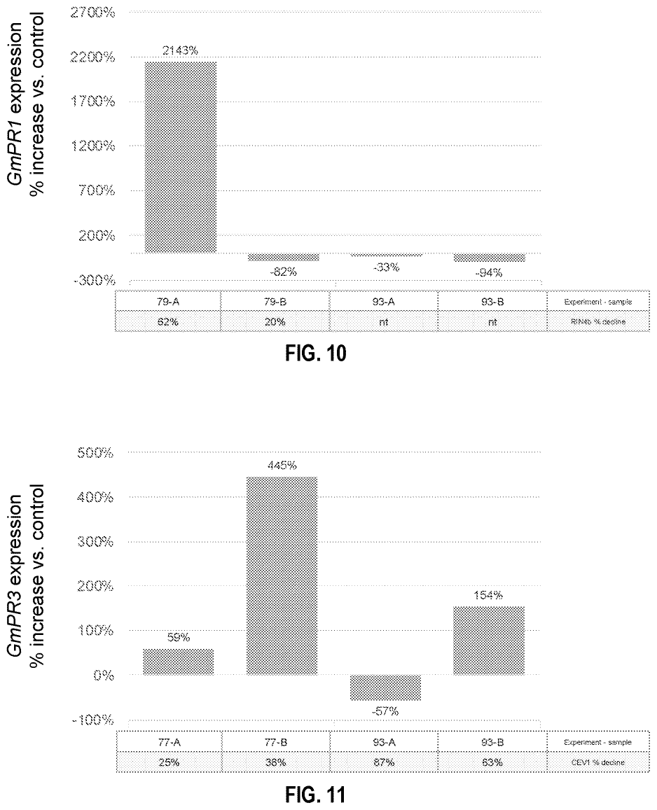

FIG. 10 shows the increased GmPR1 expression as percentage increase vs. the control for GS2109/GmRIN4b. The control LD45-average between two independent samples, A and B, was used for the comparison for sample 79 and a single pooled group for sample 93. The seed soak method from Example 2 was used with the formulation of CQD3 and the normalizer GmActin. The secondary leaf, sample 79 was day 21 post treatment and sample 93 was day 22 post treatment. Sample 93 groups were not tested (nt) for RIN4b expression as no significant increase in GmPR1 was detected.

FIG. 11 shows the increased GmPR3 expression as percentage increase vs. the control for GS279/GmCEV1. control LD45-average between two independent samples, A and B, was used for the comparison for sample 77 and a single pooled group for sample 93. The seed soak method from Example 2 was used with the formulation of CQD3 and normalizer GmActin. The secondary leaf, sample 77 was day 28 post treatment and sample 93 was day 22 post treatment.

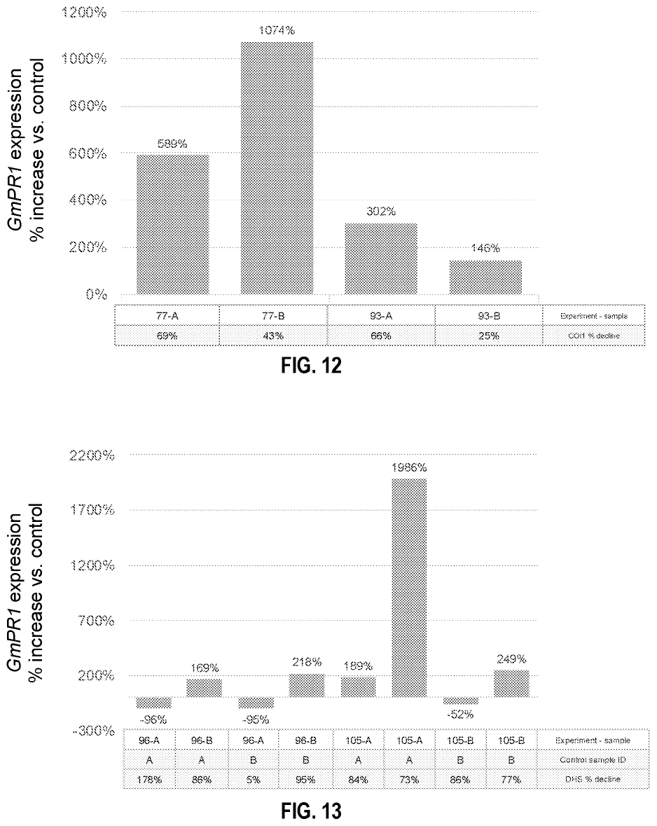

FIG. 12 shows the increased GmPR1 expression as percentage increase vs. the control for GS280/GmCOI1. The control LD45-average between two independent samples, A and B, was used for the comparison of sample 77 and a single pooled group for sample 93. The seed soak method from Example 2 was used with the formulation of CQD3 and normalizer GmActin. The secondary leaf, sample 77 was day 28 post treatment and sample 93 was day 22 post treatment.

FIG. 13 shows the increased GmPR1 expression as percentage increase vs. the control for GS415/GmDHS. The control LD45 was used in addition to the seed soak method from Example 2 was used with the formulation of CQD3 and normalizer GmActin. The secondary leaf, sample 96 was day 19 post treatment and sample 105 was day 15 post treatment.

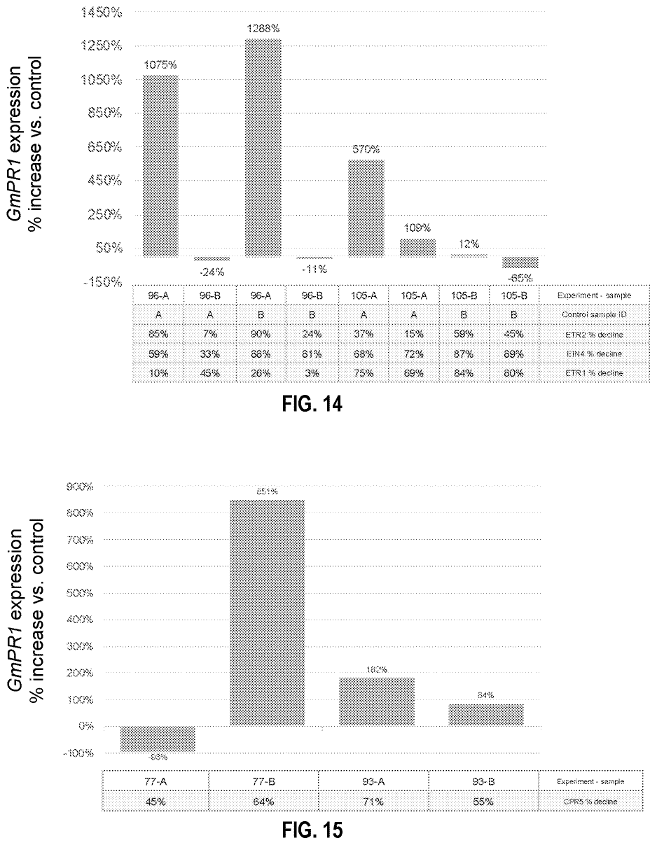

FIG. 14 shows the increased GmPR1 expression as percentage increase vs. the control for GS289/GmETR1:GmEIN4:GmETR2. The control LD45 as used in addition to the seed soak method with the formulation of CQD3 and normalizer GmActin. The secondary leaf, sample 96 was day 19 post treatment and sample 105 was day 15 post treatment.

FIG. 15 shows the increased GmPR1 expression as percentage increase vs. the control for GS275/GmCPR5. The control LD45-average between two independent samples, A and B, was used for the comparison for sample 77 and a single pooled group for sample 93. The seed soak method from Example 2 was used with the formulation of CQD3 and normalizer GmActin. The secondary leaf, sample 77 was day 28 post treatment and sample 93 was day 22 post treatment.

Collectively, these data show that dsRNA molecules that target SAR pathway suppressor genes for inhibition provide a benefit to soybean plants through induction of PR1 gene expression, which is induced in response to a variety of pathogens. PR1 gene expression is a useful molecular marker for the SAR response.

### Example 6. Increase in Defense Genes of Spinach Plants by Delivery of dsRNA

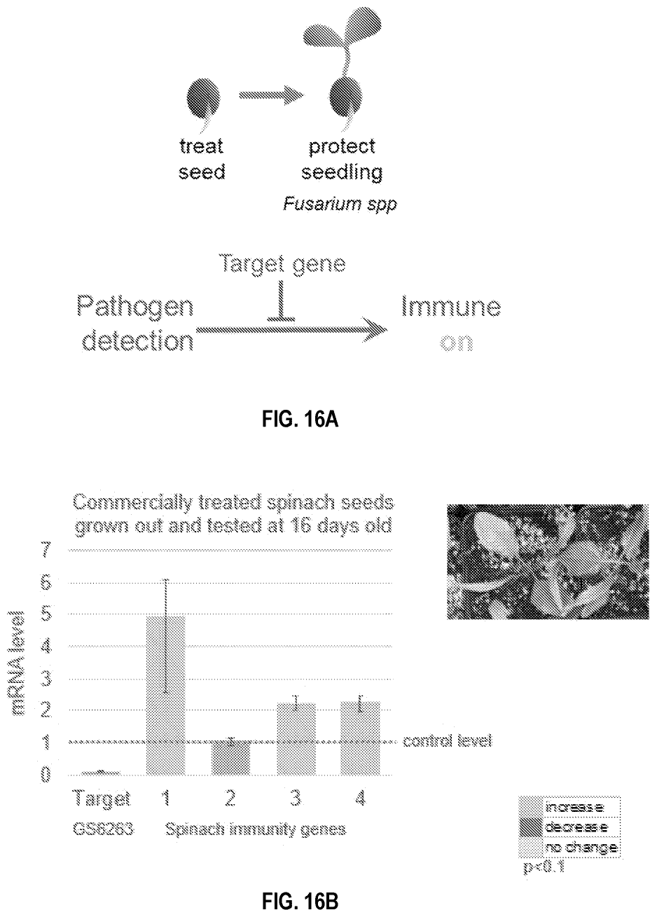

A dsRNA molecule targeting a plant gene involved in suppression of a systemic or localized protective response pathway can increase the expression of spinach defense genes. A dsRNA targeting SoWRKY33 (GS6263) was delivered to spinach seedlings by a commercial priming method wherein individual lots of spinach undergo a pretest to determine the amount of priming moisture required to prime the seed without causing the seed to germinate. A typical example may be holding the seed at 33% moisture content at 20° C. for seven days. After the primed state is achieved in the presence in dsRNA seeds are dried back down to typical seed-shipping moisture content before being planted in soil pots and allowed to grow for 16 days in the greenhouse at a range of 21° C. to 25° C. day (15 hours) and 20° C. to 21° C. night (9 hours). After 16 days, the mRNA expression levels of four spinach immunity genes and the target SoWRKY33 were determined by qRT-PCR. It was found that the relative expression of three of the four immunity genes was increased in these treated spinach plants, while the relative expression of SoWRKY33 was decreased. See FIG. 16B.

These data demonstrate that delivery of dsRNA molecules targeting plant gene involved in suppression of a systemic or localized protective response pathway can increase the expression of genes in the defense pathway of spinach plants (e.g., by decreasing expression of the target gene and ultimately increasing expression of pathogen protection genes).

### Example 7. Improvement of Health of Lettuce Plants by Delivery of dsRNA

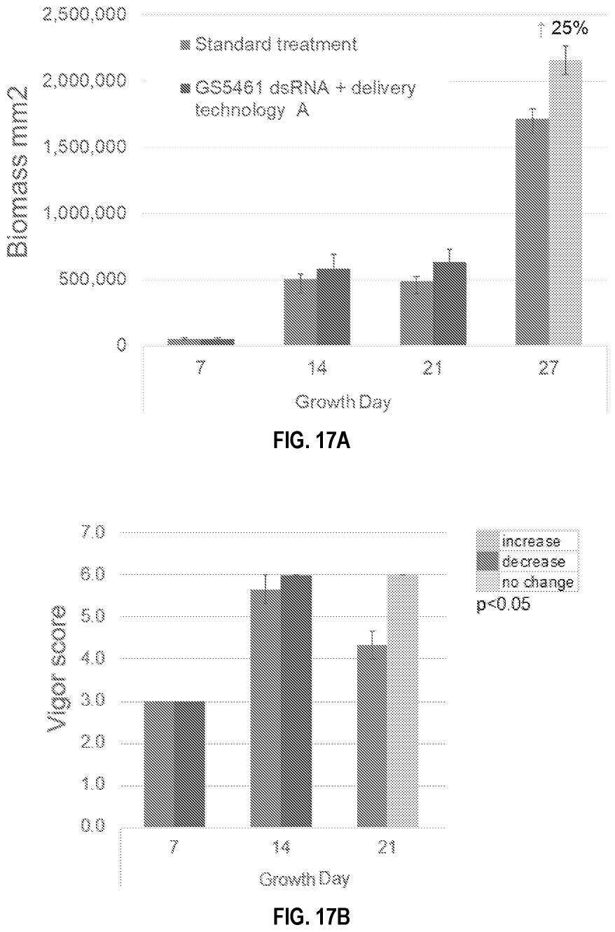

A dsRNA molecule targeting a plant gene involved in suppression of a systemic or localized protective response pathway can improve the health of lettuce plants. A dsRNA targeting LsCPR5 (GS5461) was delivered to lettuce plant seedlings by a commercial seed priming method wherein individual seed lots of lettuce are evaluated through a pretest to see if the seed is a photo or thermal dormant and using results to mathematically determine the priming temperature, duration, and biological additives needed to prime the specific seed lot to achieve a primed state. A typical example is priming lettuce at 15 C for seven hours in a priming solution containing a formulation of natural plant growth regulators while exposing the seed to red light of a narrow spectrum. After a primed state was achieved in the presence of dsRNA the seeds were dried back, planted in soil and allowed to grow for 27 days at 28 C/20 C Day/night temps 12/12 photoperiod in a Fusarium chamber (i.e., a fungal pathogen environment). Fusarium oxysporum inoculum is prepared by removing discs from a Fusarium oxysporum colony on potato dextrose agar and creating a powder inoculum that is characterized for CFU per gram. For lettuce soil inoculation a target of 1000 CFU per gram of soil is created. A control is included in which the lettuce seeds are treated with a standard pesticide treatment. At days 7, 14, 21, and 27 days, the biomass (FIG. 17A) and vigor (FIG. 17B) of the plants were determined.

It was found that the dsRNA-treated plants had improved biomass and vigor by 27 days following initial treatment. These data demonstrate that delivery of dsRNA molecules targeting plant gene involved in suppression of a systemic or localized protective response pathway can improve the health of lettuce plants (e.g., by suppressing the expression of the gene and thereby triggering the activation or increased activity of the protective response).

### Example 8. Field Trials of dsRNA

A dsRNA molecule targeting a plant gene involved in suppression of a systemic or localized protective response pathway can improve the health of lettuce plants. A dsRNA targeting LsCPR5 (GS5461) was delivered to lettuce plant seedlings (three different varietals) and allowed to grow in one of two field locations in Yuma Arizona in a random complete block design. The method of dsRNA delivery was as described in Example 7. The control was a standard priming treatment that did not include a fungicide (UTC.). Lettuce plants were challenged by naturally occurring Fusarium pathogen in the fields.

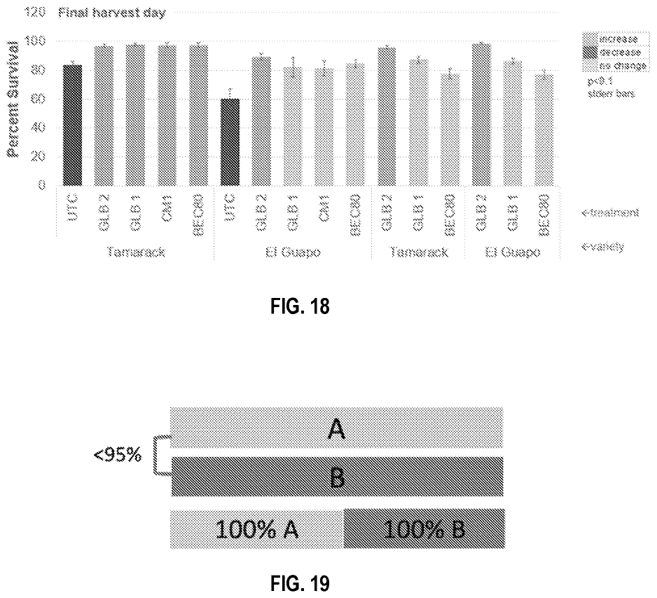

At final harvest day, 86 days after planting, the percent survival of the plants was determined by manual count of plots (FIG. 18). It was found that the dsRNA-treated plants had improved survival compared to plants treated with the standard priming method without a fungicide. These data demonstrate that delivery of dsRNA molecules targeting plant gene involved in suppression of a systemic or localized protective response pathway can improve the health of lettuce plants (e.g., by increasing expression of pathogen immunity genes) in the field.

### Example 9 dsRNA Targeting Genes Involved in Suppression of Pathogen Defense Pathways Protects Plant Seedlings from Fungal Pathogens

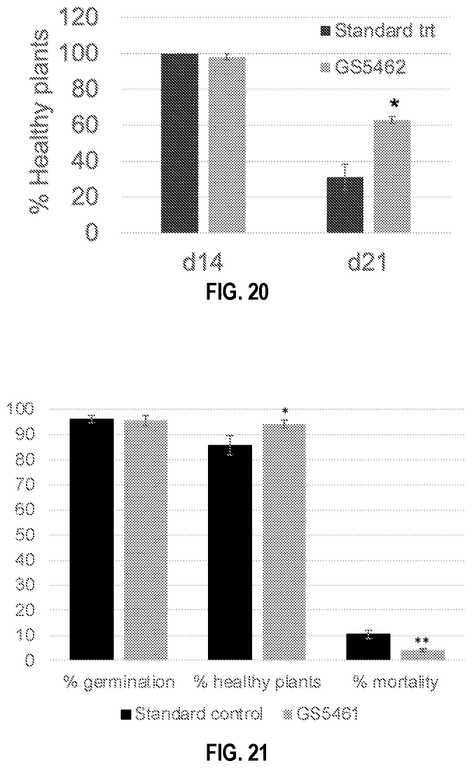

A dsRNA targeting SoCPR5 (GS5462) was applied as a seed treatment as described in Example 6 for spinach seedlings and protected the seedlings from Fusarium oxysporum infection (FIG. 20). After a primed state was achieved in the presence of dsRNA the seeds were dried back, planted in soil and allowed to grow for 27 days at 28° C./20° C. Day/night temps 12/12 photoperiod in a Fusarium chamber (i.e., a fungal pathogen environment). Fusarium oxysporum inoculum is prepared by removing discs from a Fusarium oxysporum colony on potato dextrose agar and creating a powder inoculum that is characterized for CFU per gram. For spinach soil inoculation a target of 500 CFU per gram of soil is created. A control is included in which the spinach seeds are treated with a standard pesticide treatment. At days 7, 14, and 21, the number of healthy plants are counted and percent of population determined. Results are shown in FIG. 20. Treatment with GS5462 dsRNA resulted in equivalent survival of spinach plants in the Fusarium challenge, compared to the standard treatment at 14 days and demonstrated a significant (*p<0.12) improvement in survival at 21 days.

TABLE A

| | Gene | | | | | |
|---|---|---|---|---|---|---|
| Trigger | Target | Target | PR1 | HARB | ZF | bHLH |
| GS6297 | RPLK3 | 0.27 | 0.82 | 1.03 | 0.47 | 0.83 |
| GS6300 (fusion) | DHS-1 | 0.60 | 0.68 | 1.71 | 1.59 | 1.15 |
| GS6300 (fusion) | DHS-2 | 0.45 | 0.68 | 1.71 | 1.59 | 1.15 |
| GS6296 | MPK4a | 0.50 | 1.43 | 1.63 | 0.63 | 0.45 |
| GS6291 | EIN4 | 0.60 | 1.14 | 0.81 | 0.58 | 0.63 |
| GS6285 | DHS | 1.49 | 2.38 | 1.36 | 1.26 | 1.63 |
| GS6288 | meIF-5A | 1.89 | 1.72 | 1.44 | 1.62 | 2.32 |
| GS6283 | COI1 | 2.01 | 0.78 | 2.17 | 2.77 | 2.58 |
| GS6282 | CPR5 | 3.43 | 1.39 | 3.12 | 2.80 | 3.30 |
| GS6281 | CEV1 | 3.94 | 1.73 | 2.80 | 2.83 | 3.12 |

Table A shows the fold change in gene expression for target gene and four defense associated genes when including dsRNA targeting a series of genes in a spinach seed priming treatment as compared to inclusion of a non-targeting dsRNA control.

### Example 10. DsRNA Targeting Genes Involved in Suppression of Pathogen Defense Pathways can Impact a Set of Representative Defense Genes

A series of dsRNAs targeting genes controlling plant defense response were applied as a spinach seed treatment as described in Example 6 and resulted in significant changes in defense pathway gene expression Delivery of dsRNA was the same as in Example 9. Plant growth was the same as for Example 6. Spinach leaf tissue was sampled at 16 d post planting. Results are shown in Table 2. Bold numbers statistically significant (p<0.1). Expression of the target gene for each trigger sequence was measured by qRT-PCR and fold-change vs. non-targeting control dsRNA is shown in the Target column. Several triggers resulted in a statistically significant downregulation of their respective target genes. Significant increases in target gene expression may be due to a negative feedback loop of self-regulation that manifests over the 16 days of growth potentially as an oscillating wave across time. Gene expression was also measured for markers of defense responses, specifically PR1, HARB, ZF, and bHLH genes. The column labeled with each gene name shows fold change compared to non-target control. Where a dash is placed after a target gene name this indicates a paralog of the target gene and expression assayed by qRT-PCR for the specific paralog.

### Example 11. Improvement of Health and Reduction in Mortality of Lettuce Plants by Delivery of dsRNA

A dsRNA molecule targeting a plant gene involved in suppression of a systemic or localized protective response pathway can improve the health of lettuce plants. A dsRNA targeting LsCPR5 (GS5461) was delivered to lettuce plant seedlings as in Example 7. After a primed state was achieved in the presence of dsRNA including a formulant CQD3, the seeds were dried back, planted in soil and allowed to grow until soil emergence at 20 C/20 C day/night then thereafter at 28 C/20 C Day/night temps 12/12 photoperiod in a Fusarium chamber (i.e., a fungal pathogen environment). Fusarium soil inoculation was conducted as in Example 7. Three sets of 60 plants per treatment were scored for germination, health of individual plants and for plant death weekly for 35 days.

It was found that the dsRNA-treated plants had improved percentage of healthy plants and reduced mortality by day 35. Results are shown in FIG. 21. These results demonstrate that delivery of dsRNA molecules targeting plant gene CPR5 involved in suppression of a systemic or localized protective response pathway improves the health of lettuce plants and reduce mortality. (e.g., by suppressing the expression of the gene and thereby triggering the activation or increased activity of the protective response).

### Example 12 Increase in a Defense Gene of Lettuce Plants by Delivery of dsRNA

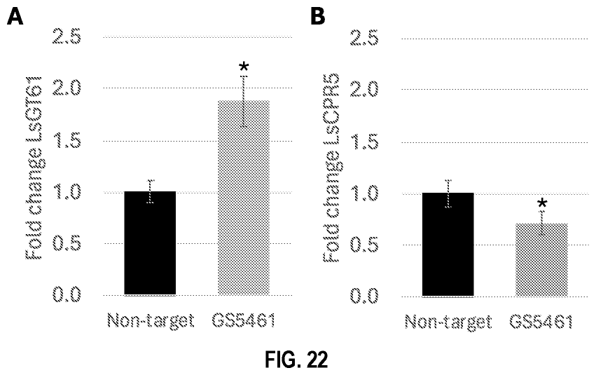

A dsRNA molecule targeting a plant gene involved in suppression of a systemic or localized protective response pathway can increase the expression of a lettuce defense gene. A dsRNA targeting LsCPR5 (GS5461) or non-targeting control were delivered to lettuce seedlings by a commercial priming method as described in Example 7 and grown in a greenhouse as described in Example 6. After 14 days, the mRNA expression levels of a lettuce immunity gene, LsGT61, and the target LsCPR5 were determined by qRT-PCR. It was found that compared to a non-targeting dsRNA control the expression of LsGT61 was increased while the expression of LsCPR5 was decreased *p<0.05 (presumably due to direct inhibition by the dsRNA). See FIG. 22.

These data demonstrate that delivery of dsRNA molecules targeting plant gene involved in suppression of a systemic or localized protective response pathway can increase the expression of genes in the defense pathway of lettuce plants (e.g., by decreasing expression of the target gene and ultimately increasing expression of pathogen protection genes).

TABLE 1
Double-stranded RNA sequences

| | | | SEQ | | | |
|---|---|---|---|---|---|---|
| | | | ID NO | | | SEQ ID |
| dsRNA | Target | Gene | (Gene | | | NO |
| Name | ID | Target | Target) | Species | dsRNA sequence | (dsRNA) |
| GS6434 | AT4G01370 | AtMPK4 | 1 | Arabidopsis | TGGAGAGGAGGTAGCTATCAAGAAGATTGGTAATGCTTT | 87 |
| | | | | thaliana | TGACAATATTATCGATGCTAAAAGGACTTTGAGAGAGAT | |
| | | | | | TAAGCTTCTCAAGCATATGGATCATGAAAATGTGATTGC | |
| | | | | | TGTAAAGGATATAATAAAACCACCGCAGAGAGAGAACT | |
| | | | | | TCAATGATGTTTACATTGTTTATGAGCTTATGGACACTG | |
| | | | | | ATCTCCACCAAATTATTCGCTCTAACCAACCCTTAACTG | |
| | | | | | ATGATCATTGTCGGTTTTTTCTGTATCAGTTGTTGCGTGG | |
| | | | | | GCTCAAATATGTTCATTCAGCTAATGTATTGCATCGAGA | |
| | | | | | TTTGAAACCTAGCAAT | |
| GS6435 | AT5G64930 | AtCPR5 | 2 | Arabidopsis | TTGATGAATGAGCTTGCCCTTTTCGAAGAGACTCTACAA | 88 |
| | | | | thaliana | CTCTCTTGTGTCCCTCCTAGAAGTTCAGCAATGGCTTTGA | |
| | | | | | CCACAGACGAAAGGTTTTTAAAAGAGCAAACACGAGCA | |
| | | | | | AACGACCTAAAGACCGTGGAGATTGGTCTTCAAATAAG | |
| | | | | | AGAGTTAAGGTGCAAAGAGACGGCGCTAGGATTAAAAT | |
| | | | | | TTGAATCAAACAACCTGGGGAAAGCGGCGCTAGAGTTG | |
| | | | | | GATGTTTCGAAAGCTGCATTCAGAGCGGAGAAATTCAA | |
| | | | | | AACCGAATTAGAAGATACAAGAAAAGAAGAGATGGTCA | |
| | | | | | CAAGAATCATGGATT | |
| GS200 | Glyma.1 | GmMPK4a | 3 | Glycine max | CTGTATCAATTGTTACGAGGGCTCAAATATGTACATTCA | 89 |
| | 6G032900.1 | | | | GCAAATGTTTTGCACCGAGATCTAAAGCCTAGTAATTGC | |
| | | | | | TATTGAATGCCAATTGTGACCTTAAGATTGCGGACTTTG | |
| | | | | | GTCTTGCTAGAACCACATCTGAAACTGACTTTATGACTG | |
| | | | | | AGTATGTGGTCACTAGATGGTACCGAGCTCCCGAATTGC | |
| | | | | | TTCTTAATTGTTCAGAATATACAGCAGCCATTGATATAT | |
| | | | | | GGTCTGTTGGTTGCATACTTGGTGAAATCATAACAAGAC | |
| | | | | | AACCGCTCTTTCCTGGCAAAGATTATGTTC | |
| GS201 | Glyma.0 | GmRLK3 | 4 | Glycine max | ATTATCTCTCTGGAGAGTTTCCTGAGGTGGGTTCTGTTGC | 90 |
| | 1G197800.1 | | | | TGTTAATCTTGGTCAGATTACTCTCTCTAACAACCAGCTT | |
| | | | | | TCTGGGGTTCTACCTCCCTCCATTGGTAACTTCTCCAGCG | |
| | | | | | TGCAGAAGCTCATTCTTGATGGCAACATGTTCACGGGTC | |
| | | | | | GGATACCTCCCCAGATTGGGAGGTTGCAACAGCTTTCTA | |
| | | | | | AGATTGATTTTAGTGGCAACAAGTTCTCGGGTCCTATTG | |
| | | | | | TGCCTGAGATCAGTCAGTGTA | |
| GS275 | XM_014 | GmCPR5 | 5 | Glycine max | AGGAATCTTCCTCCTGGTGGACTCCCAAGTCAATGGTCT | 91 |
| | 776442.2 | | | | TATTCAATTCAAAGTTGCATATTTTGTGGTGTCAAGTTCA | |
| | | | | | AGTTATGAGCCGAATGGCATTTGGCATCTTGATGATTTT | |
| | | | | | TGCTGTTGCATATTTGCTCTTGCAGCGGTCGACCACAAC | |
| | | | | | ATCACAGACTATGCCAGTCACTTTCATCCTTTTAATGTTG | |
| | | | | | GGAATTTTTTGTGGCTATTGTGGCAAGCTATGTGTAGAG | |
| | | | | | ACATTGGGTGGGAGTGGTATTGTGTGGCTCTTATACTGG | |
| | | | | | GAGATTATGTGCTTGCTGCATTTCTTA | |
| GS276 | XM_014 | GmNPR3 | 6 | Glycine max | AGTGTTCATCGATGTATTCTGGCCTCTAGGAGTAAGTTTT | 92 |
| | 767311.2 | | | | TCCATGAATTATTCAAGAGAGAGAAGGGTTCATCAGAA | |
| | | | | | AAAGAAGGGAAATTGAAGTATAACATGAGTGATTTGTT | |
| | | | | | GCCTTATGGCAAGGTTGGATATGAAGCCTTCCCCATATT | |
| | | | | | CCTTGGCTATGTATATACTGGTAAACTCAAGCCCTCTCC | |
| | | | | | AATGGAAGTGTCTACTTGTGTTGACAGTGTGTGTGCCCA | |
| | | | | | TGATGCTTGTAGACCTGCCATTAACTTTGCTGTGGAGTT | |
| | | | | | GATGTATGCCTCTTACATTTTTCAAATACCAGAGTTTGTA | |
| | | | | | TCACTTTTCCAGAGACGTCTACTTAACTTTATAGGGAAG | |
| | | | | | GCTCTTGTGGAAGATGTCATCCCAATCCTCACTGTTGCTT | |
| | | | | | TCCATTGTCAATTGAGTCAACTTGTCAATCAATGCATTG | |
| | | | | | ATAGGGTGGCCAGATCAGACCTTGACCAGATTTCAATTG | |
| | | | | | ACCAAGAGCTTCCAAATGAACTCTCACAAAAAGTTAAA | |
| GS277 | XM_003 | GmRFC3 | 7 | Glycine max | ATGGCGCAGACGACCCATCCCATGGACATCGACGACCA | 93 |
| | 526588.4 | | | | CACTTACACCGCCAGTCCTGACAAGGGCAAAAGTGTCGT | |
| | | | | | CTTCGCCGGAAACCCTCCCGCCGCCGGCAAGGCCATTCC | |
| | | | | | GTGGGTCGAAAAATACCGCCCTCAGTCCCTCGACGACGT | |
| | | | | | CGCTGCTCACCGTGACATTGTCGATACAATTGATAGGCT | |
| | | | | | AACCACTGAGAACAGATTGCCCCATCTTCTGTTGTATGG | |
| | | | | | CCCTCCTGGCACGGGCAAAACATCAACGATTCTTGCTGT | |
| | | | | | GGCACGCAAACTGTATGGTTCACAGTACCAAAATATGAT | |
| | | | | | TTTGGAGCTCAATGCATCCGATGATAGAGGAATTGATGT | |
| GS278 | XM_006 | GmSNI1 | 8 | Glycine max | CACTTCCCTCGCGTGTATTTATCTGGCACGGATGATTCCC | 94 |
| | 583777.3 | | | | GATCATCATCTAACTCTCCATCAAAATTGGTTGTGGTTA | |
| | | | | | AAGAGGCATGGTCTCCCATTATCGATTTTGTGGATAAGG | |
| | | | | | CCACTGCTGTTAGTGAAGCTGGTGATAAACAGTCTGGTG | |
| | | | | | GATCTCTTGACCCCTCTAGCTTTCAAGTCTTGATTGAAG | |
| | | | | | GGCTGGCTGAAGTTTTAGCAGAGACCAAATTTGAAGCA | |
| | | | | | GCAAGCATGGAGCCCTTGAGGAACATGTTAATCTTTCAG | |
| | | | | | TACCTTGTCGTTGTTTTTGAGGATGATTTTTTACCTCGCA | |
| | | | | | ATGCAACCTTGAACTGGAGCATGCAGAGGGAGTCTTTGC | |
| | | | | | TCAGCCTGCTACTG | |
| GS279 | XM_006 | GmCEV1 | 9 | Glycine max | CAGTCCTTACCAATGGACCTTTCTTATTGAATCTTGATTG | 95 |
| | 594350.3 | | | | TGATCACTACATAAACAACAGCAAGGCCTTGAGGGAAG | |
| | | | | | CTATGTGCTTTATGATGGATCCAAACCTTGGTAAACATG | |
| | | | | | TTTGCTATGTCCAGTTTCCACAGAGGTTTGATGGTATTGA | |
| | | | | | TAGGAATGATCGATATGCCAATCGTAATACAGTTTTCTT | |
| | | | | | TGATATAAACTTGAGAGGTTTGGATGGCATTCAAGGTCC | |
| | | | | | TGTTTATGTGGGTACTGGATGTGTCTTCAATAGGACAGC | |
| | | | | | TTTATATGGTTATGAACCTCCTCTTAAACCCAAGCACAA | |
| | | | | | AAAGCCTGGGTTGTT | |
| GS280 | NM_001 | GmCOI1 | 10 | Glycine max | CATCCCTTACATCGACGACCCCAAGGACCGCGACGCCGT | 96 |
| | 251661.1 | | | | CTCACAGGTCTGCCGACGCTGGTACGAACTCGACTCCCT | |
| | | | | | CACTCGGAAGCACGTCACCATCGCGCTCTGCTACACCAC | |
| | | | | | CACGCCGGCGCGCCTCCGCCGCCGCTTCCCGCACCTTGA | |
| | | | | | GTCGCTCAAGCTCAAGGGCAAGCCCCGAGCAGCAATGT | |
| | | | | | TCAACTTGATACCCGAGGATTGGGGAGGCCACGTCACCC | |
| | | | | | CATGGGTCAAGGAGATTTCTCAGTACTTCGATTGCCTCA | |
| | | | | | AGAGTCTCCACTTCCGCCGTATGATTGTCAAAGATTCCG | |
| | | | | | ATCTTCGGAATCTCGCTCGTGACCGCGGCCACGTGCTTC | |
| | | | | | ACTCTCTCAAGCTTGACAAGTGCTCCGGTTTCACCACCG | |
| | | | | | ATGGTCTTTTC | |
| GS282 | XM_003 | GmRAP | 11 | Glycine max | GGCGAGCTGCTTTATCTTTCTGAAATGGATAGAATTGCA | 97 |
| | 529909.3 | | | | GAGGTTGCACTGACCAAGGTTGGGGAATTCAACTCTCAA | |
| | | | | | AACATTGCTAATATTGCAGGTGCCTTTGCTGCAATGCAG | |
| | | | | | CATTCTGCTCCTGATCTTTTCTCAGAGTTTTCAAAAAGGG | |
| | | | | | CTTCAGACATAATTCACACTTTCCAAGAACAAGAACTTG | |
| | | | | | CTCAGCTTTTGTGGGCTTTTGCTTCTCTATATGAGCCTGC | |
| | | | | | TGACCCTATATTTGATTCTTTAGACATAGTCTTCAAAGAT | |
| | | | | | CATAGCCAATTGAGAGGTTGCATAGGTGAAAAGACATC | |
| | | | | | AAATAATCATGAACAAATAAGTGTTGATAGAAGTGGTG | |
| | | | | | CCTCAAATGGATC | |
| GS283 | XM_003 | GmDMR6- | 12 | Glycine max | GTGCTGTCCAACGTAGCCTTTCAAGGGCATGCCCTCAAA | 98 |
| | 535801.3 | like | | | TATGTTTCTTCGTTTGGTTTCTGTTGGGAGATCGAACAGC | |
| | | | | | TCCTTCATGGAGCCAAATACACCATTTTGTAGCTCAATA | |
| | | | | | GAAGCTTTATCATACACAGCAACAAAGTAACCATTCTCC | |
| | | | | | TCAAATGCTTGGCGCACACTTGTGCATGTTGATAACCAG | |
| | | | | | GAATTGGTCCCTGGCTTCAAATCTTCTATGGTGAAATCT | |
| | | | | | AGAACAGGAAGCATAGGTTTTCCTTCAGAACCCATCGTA | |
| | | | | | TGCCT | |
| GS284 | NM_001 | GmARR9 | 13 | Glycine max | TCACAGACTATTGTATGCCCGGAATGACCGGTTATGAAT | 99 |
| | 251146.1 | | | | TACTCAAGAGAATCAAGGAATCATCTACTTTCAAGGAA | |
| | | | | | ACGCCTGTGGTTATTATGTCATCTGAAAACGTTTTGCCA | |
| | | | | | CGCATAGACAGATGTTTGGAGGAGGGTGCAGAGGATTT | |
| | | | | | CATAGTGAAGCCAGTGAAGTTATCTGATGTGAAGCGTTT | |
| | | | | | GAAGGATT | |
| GS285 | XM_003 | GmNPR4 | 14 | Glycine max | TAAGAAACATGAAGAACTGTTGAAGGACGCAGAGAAAT | 100 |
| | 544249.1 | | | | GGGCAAAGGAGACGGCAACATCCTTCACAATAGTGGGG | |
| | | | | | ACTCTTATAATAACCATTGTGTTTGCAGCAGCCTTCACTC | |
| | | | | | TTCCTGGAGGAAATGATCAAAATACTGGTATTCCTATGT | |
| | | | | | TTTTGCACAAAAGAATGTTCACAACTTATATGGTGGCTG | |
| | | | | | ATGCTATCTCACTCTTTTCTTCCTCCACTGCTGTGATGAC | |
| | | | | | TTTTATTGGAATTCTCACATCTCGTTATGCTGAAAGAGAT | |
| | | | | | TTCCTTAAATCCTTGCCACTCAAGTTGATGTTTGGCCTTT | |
| | | | | | TCACACTCATATGTTCTATCTTGGCCATGATGGTTGCATT | |
| | | | | | TTGCTCTGCCTTTTCTTTGATGCTTGAGGATTCGGGTCAT | |
| | | | | | TCCAAGATGGTAAAATTTGTCATTTCCATTGCAAGCTTA | |
| | | | | | TCTGTGGTCA | |
| GS286 | XM_003 | GmNAC25 | 15 | Glycine max | GGGTATTGGAAGGACACAGGTGTCACGGAACCTATATT | 101 |
| | 523175.4 | | | | GTCAACCTTTGATAAGAAAGTGGGAATGAAGAAGTACT | |
| | | | | | TGGTGTTCCACATAGGTGAAGCTCCACAAGGTACTGAAA | |
| | | | | | CCAGTTGGGTAATGCAAGAATATTATATATGTTCCTCTG | |
| | | | | | GGTTTGACACTGCCTCATATCGAAGTGCTAGGAGAAGA | |
| | | | | | AGAAAACATGATCAAAGTTGGAGCAAGTGGGTTTTGTG | |
| | | | | | CAAAGTCTATGAGAAGATCAGGTCTCAACAAGGTGTAA | |
| | | | | | ACTGCTATAGCGATGATGATGACAGTGG | |
| GS287 | NM_001 | GmBRIla; | 16 | Glycine max | GCTTACTTTTGCGGACCTTCTTGACGCTACCAATGGCTTT | 102 |
| | 359015. | GmBRI1b | | | CACAATGATAGTCTCATTGGCTCTGGTGGGTTTGGGGAT | |
| | 1; NM_0 | | | | GTTTACAAGGCTCAGTTGAAGGATGGAAGTGTTGTGGCT | |
| | 0125048 | | | | ATCAAGAAGCTGATTCATGTCAGCGGCCAAGGGGACAG | |
| | 2.2 | | | | GGAATTCACTGCTGAAATGGAGACCATTGGGAAGATCA | |
| | | | | | AGCACAGGAACCTTGTTCCCCTGTTGGGATACTGCAAGG | |
| | | | | | TAGGGGAAGAGAGGCTCTTGGTTTATGAGTACATGAAAT | |
| | | | | | ATGGAAGTTTAGAGGATGTTCTACATGATCAGAAGAAA | |
| | | | | | GCTGGGATCAAGCTGAACTGGGCCATTAGGCGGAAAAT | |
| | | | | | TGCTATTGGAGC | |
| GS288 | XM_014 | GmTAR2; | 17 | Glycine max | GCATAGGGTGGGCTATTGTCAAAGATAAAGAGGTAGCA | 103 |
| | 775454. | GmYUC2a | | | AAGAAAATGACTAAATTCATAGAGATAAGTACAATCGG | |
| | 2; XM_0 | | | | TGTCTCGAAGGATTCTCAACTCAGAGCTGCCAAGGTTTT | |
| | 0353334 | | | | AAAGGCAGTGTCTGACAGCTGCGAACAAGAAAACTCCC | |
| | 0.4 | | | | AACAAGTGGTTCCCCATACGTTTTGTGGATCAATTTTTG | |
| | | | | | CTTCTAATGTCACATCTCATGCTTGGAGACACAGCTCAA | |
| | | | | | TTTGGACTTCGTCGTCCAAAACTTGGTCCTCTAGAGCTC | |
| | | | | | AAGAACTTGTACGGAAAGACACCAGTTTTGGATGTTGG | |
| | | | | | GA | |
| GS289 | XM_006 | GmETR1;E | 18 | Glycine max | ACTGCTGTGGTATCGTGTGCAACTGCCCTCATGCTGGTA | 104 |
| | 586685. | IN4;ETR2 | | | CATATTATTCCTGATTTATTAAGTGTTAAAACTAGGGAA | |
| | 3; XM_0 | | | | TTATTTTTGAAGAACAAGGCTGCAGAGCTTGATAGAGAA | |
| | 1477403 | | | | ATGGGCCTGATTCGCACACAAGAAGAAACTGGTTGAGG | |
| | 8.2; XM_ | | | | GAAGCTTCTTGCACTGCCAAGTGTTTGTGTATATATAAA | |
| | 0147727 | | | | GGCTTTGGTCTTGAAGTTGATGTTGACAAGTCTTTGCCTG | |
| | 85.2 | | | | ATCTGGTCATTGGTGATGAGGCAAGGACTTTCCAAGTAA | |
| | | | | | TAGGGTAGTCCAGTTGATGCAGGGGAACATATGGTTAGT | |
| | | | | | ACCTTGTAATCATGGTTTTCCTCAAAGCATGACTCTTCTT | |
| | | | | | CTTCGGTTTCAATTACGACCGTCCATTTCCATAGCCATCT | |
| | | | | | CTGACCC | |
| GS423 | Glyma.0 | GmACS1 | 19 | Glycine max | ATTATCATGGTCTGCCCGAGTTCAGAAATGCTGTGGCTA | 105 |
| | 5G211700.1 | | | | AATTCATGGGTAGAACAAGAGGAAACAGAGTCACGTTT | |
| | | | | | GATCCTGACCGTATTGTCATGAGCGGTGGAGCAACTGGA | |
| | | | | | GCACACGAAGTCACTACCTTTTGTTTGGCAGACCCTGGT | |
| | | | | | GACGCATTTTTGGTGCCCATTCCTTATTATCCAGGTTTTG | |
| | | | | | ACCGGGATTTGAGGTGGAGAACAGGAATTAAACTTGTT | |
| | | | | | CCAGTTATGTGCGATAGCTCAAACAATTTCAAGTTGACA | |
| | | | | | AAGCAAGCATTGGAAGATGCGTATGAGAAGGCCAAAGA | |
| | | | | | GGATAATATAAGAGTAAAGGGCTTGCTCATCACCAATCC | |
| | | | | | ATCAAACCCATTAGGCAC | |
| GS436 | Glyma | GmCRY1b; | 20 | Glycine max | TGAACCATGGGATGTGAATGATGCACATGGCCGACCATT | 106 |
| | 06G103 | GmCRY2 | | | CACAACTTTTGCTGCTTTTTGGGAAAGATGTCTTAGCAT | |
| | 200.1; Gl | | | | GCCTTATGACCCTGAGTCACCTCTTCTCCCACCTAAAAG | |
| | yma_10 | | | | GATAATTCCAGGTGATGCATCGAGGTGCCCTAGTGATAC | |
| | G180600 | | | | ATTGCTGTTTGAAGATGAATTAGAGAAGGCAAGCAATG | |
| | | | | | CACTTGCACTTCTGGGGCACTTAAAGTTTTTTCCTTGGAA | |
| | | | | | TCCTGATCCTGATATCTTTAAGACTTGGAGACAAGGTAG | |
| | | | | | GACTGGCTTTCCTTTGGTTGATGCAGGAATGAGAGAACT | |
| | | | | | TTGGGCAACAGGATGGATACACAACAGAATAAGAGTAA | |
| | | | | | TAGTTTCCAGTTTTGCTGTGAAAATGTTGCTTCTACCCTG | |
| | | | | | GAAATGG | |
| GS437 | Glyma.0 | GmACO4 | 21 | Glycine max | AATGGTGACCAGCAAAGGTCTTGAGTCAGTTCAGTCAG | 107 |
| | 9G0084 | family | | | AAATCAATGACTTGGACTGGGAAAGCACCTTTTTCCTGC | |
| | 00.1; Gly | | | | GCCATCTTCCACTCTCCAACGTTTCAGACAACGCAGATC | |
| | ma.17G0 | | | | TTGATCAAGATTACAGGAAGACAATGAAGAAATTTGCT | |
| | 09800.1 | | | | CTGGAACTGGAGAAACTTGCAGAGCAGCATGGAGCAAA | |
| | | | | | GGTTCCAAGAAATGGTAGCAAGCAAAGGCTTGGAGTCT | |
| | | | | | GCTCAATCTGAAATCAATGATTTGGACTGGGAAAGCACT | |
| | | | | | TTCTTTTTGCGCCATCTTCCTGTCTCTAACATCTCAGAGA | |
| | | | | | TCCCTGATCTTGATGAAGATTACAGGAAGGTAATGAAG | |
| | | | | | GATTTTGCCGTGGAACTGGAGAAACTGGC | |
| GS438 | Glyma.1 | GmCTR1_f | 22 | Glycine max | AACGTTTACGGCATCCAAACATTGTTTTGTTTATGGGGG | 108 |
| | 3G1511 | amily | | | CAGTCACTCAGCCTCCTAACTTATCAATTGTCACGGAAT | |
| | 00.1; Gly | | | | ATTTATCTAGGGGTAGCTTATATAGGCTTTTGCATAGAT | |
| | ma.02G1 | | | | CTGGCGCCAAAGAAGTATTGGATGAGAGGCGTAGGCTT | |
| | 65800.1 | | | | GGTATGGCTTATGACGTGGCAAAGGGGATGAATTACTG | |
| | | | | | AAGCTCTGGGTGCAAATTATGCGGGTGGGAACAGCCAT | |
| | | | | | CTGATTGCTTTGATTCCTGGTGCAGAGGAATATGAGTCT | |
| | | | | | TTCAATGAAGCAAACCAGTCAATAATGGATTACCCAAGT | |
| | | | | | CATGAAGTTGATCTTGACAAGGAGGATCTGGATATTCCA | |
| | | | | | TGGAGTGAACTTATTTTGAAGGAGAACATTGGGACAGG | |
| | | | | | GTCA | |
| GS2108 | XM_006 | GmMPK6 | 23 | Glycine max | CCAGAGCTTCTGTTGAACTCTTCTGATTACACTGCAGCA | 109 |
| | 574411.3 | | | | ATTGATGTATGGTCTGTTGGTTGTATTTTCATGGAACTGA | |
| | | | | | TGGATCGCAAGCCTTTGTTTCCTGGCAGAGATCATGTGC | |
| | | | | | ATCAGTTGCGTCTACTTATGGAGCTGATTGGCACCCCAT | |
| | | | | | CAGAGGCTGATTTGGGGTTCCTGAATGAAAATGCTAAG | |
| | | | | | AGATACATTAGGCAACTACCCCTTTACCGCCGTCAATCT | |
| | | | | | TTCCAGGAAAAGTTTCCACATGTCCATCCTGAAGCTATA | |
| | | | | | GATCTTGTTGAAAAAATGTTAACTTTTGATCCTAGAAAA | |
| | | | | | AGGATTACTGTTGAAGATGCACTGGCGCACCCGTACTTG | |
| | | | | | ACATCTCTGCATGACATCAGTGATGAACCAGTGTGCATG | |
| | | | | | ACTCCCTTCAACTTTGACTTTGAACAACATG | |
| GS2109 | NM_001 | GmRIN4b | 24 | Glycine max | CTACCTCCTACCAGACCTAGAACTAATTCAGAGGATCAA | 110 |
| | 253044.2 | | | | TCCGGAAAAGGATCACTGCATTTGGAAGATGATCCCAA | |
| | | | | | GAATTTTATCGAATCTCCAGCTCGTCATGACAATGTAAG | |
| | | | | | CAGTAGGAGTGGCAGTAGAAGCCATGGAGTAGGTTCTG | |
| | | | | | CTGATAACCGTAGAAGACATTCAACGCAAAGTACTGGG | |
| | | | | | TCTGAGTACAGCATTGAACGTTCACCGCTTCATCGCCAG | |
| | | | | | GCTAGAGCCCCA | |
| GS2110 | NM_001 | GmRIN4a | 25 | Glycine max | GGTGACTGGGATGTGAATAACCCATCATCAGCAGATGG | 111 |
| | 248292.2 | | | | CTTTACTCACATTTTCAACAAAGTGAGGGAGGAGAGAC | |
| | | | | | AGGGTGTCCCAGGACAAGTGCCGGGAACGCCTAATGAG | |
| | | | | | AGACCACAAGCTATTAGGGGCCAATCTAACGATGACAA | |
| | | | | | AGTCCAG | |
| GS274 | XM_014 | GmRapTOR | 26 | Glycine max | AAGGTTCCTTGATATGGGGCCATGGGCTGTAGATCTGGC | 112 |
| | 770914.1 | | | | ATTATCTGTTGGAATATTCCCATATGTTCTAAAGCTGTTA | |
| | | | | | CAAACAACAACACCAGAACTACGACAGATCCTTGTATTC | |
| | | | | | ATATGGACAAAGATTCTTGCACTTGATAAGTCATGTCAG | |
| | | | | | GTTGATCTAGTGAAGGATGGTGGTCATATCTATTTTATT | |
| | | | | | AAGTTTCTTGACAGCATGGAAGCATATCCAGAGCAACG | |
| | | | | | CGCAATGGCTGCTTTTGTTTTGGCTGTGATTGTGGATGGT | |
| | | | | | CATAGACGAGGCCAAGAAGCTTGTA | |
| GS281 | XM_003 | GmTOR | 27 | Glycine max | GACCCAGGCTTGGGATATTTATTATCATGTTTTCAGAAA | 113 |
| | 538286.4 | | | | GATAGATAAGCAACTTCAAAGTCTTACAACTTTAGACTT | |
| | | | | | GGAGTCTGTTTCTCCAGAATTGCTAGAATGCCGCAATTT | |
| | | | | | GGAGCTTGCTGTTCCCGGGTCATACCGTGCTGATGCACC | |
| | | | | | AGTGGTTACAATTGCATCATTTGCACGCCAACTTGTTGT | |
| | | | | | CATAACATCCAAGCAACGACCCAGGAAGTTAACCATTC | |
| | | | | | ATGGGAGTGATGGTGATGATTATGCCTTCTTATTGAAGG | |
| | | | | | GACATGAGGATTTACGCCAGGATGA | |
| GS414 | Glyma | GmGID1B; | 28 | Glycine max | AAGGGGGAGAAGCATTGAAGGACTCAAATTCCCTAAAA | 114 |
| | 03G148 | GmGID1C | | | GTCTTGTTTGTGTGGCTGGTTTGGATCTTCTGCAGGATTG | |
| | 300; Gly | | | | GCAATTGGCATATGCAAAAGGTCTTGAGGATTGTGGAC | |
| | ma_10G | | | | AACAGGTCAAGCTTCTTTTTCTCAAGGAAGCCACTATTG | |
| | 158000 | | | | GCTTCTACTTCTTGCCAAACAATGACCAGGAGAAGAACG | |
| | | | | | TTCGGTGAACATTCTTGACCTTGAGAAGCCTGTGAGCTC | |
| | | | | | TGAGGTTGTTCCTGTCATCATATTTTTCCATGGTGGAAGT | |
| | | | | | TTTGCGCATTCTTCAGCCAATAGTGCTATATATGACACC | |
| | | | | | CTTTGTCGTCGCCTAGTGGGTATCTGTAAGGCTGTTGTG | |
| | | | | | GTGTCTGTG | |
| GS415 | Glyma.0 | GmDHS | 29 | Glycine max | GCATCGCATGGTTGATGTAGTTGTTACAACTACAGGTGG | 115 |
| | 7G127600.1 | | | | CATTGAAGAAGATCTTATAAAGTGCCTGGCACCAACATA | |
| | | | | | TAAAGGAGATTTCTCTTTGCCTGGAGCATATCTGCGCTC | |
| | | | | | AAAAGGGTTGAATCGGATTGGTAATTTATTGGTCCCGAA | |
| | | | | | TGACAACTATTGCAAATTTGAGGACTGGATTATTCCTAT | |
| | | | | | CTTTGATCAGATGTTGGGGGAACAAAATAATGAGAATGT | |
| | | | | | GTTGTGGACACCATCTAAGTTAATAGCTCGATTGGGAAA | |
| | | | | | AGAAATAAAAAATGAAAGCTCTTACCTTTACTGGGCATA | |
| | | | | | CAAGAACAACATTCCAGTTTTCTGTCCGGGCTTAACTG | |
| GS416 | Glyma.1 | GmGA3OX | 30 | Glycine max | TGTGGTCTGAGGGATTCACAATTGTTGGATCCCCTCTTG | 116 |
| | 3g361700 | | | | AGCATTTTCGTCAACTCTGGCCCCAAGATTACCACAAAT | |
| | | | | | ACTGTGATATCGTCAAGCGATATGATGAAGCCATGAAA | |
| | | | | | AAGCTAGTGGGAAAGCTGATGTGGCTGATGTTGGATTCT | |
| | | | | | CTGGGTATTACAAAGGAAGACCTGAAATGGGCCGGGTC | |
| | | | | | CAAAGGCCAATTCAAAAAGACATGCGCAGCCTTGCAAT | |
| | | | | | TGAACTCTTACCCGACTTGTCCGGATCCGGATCGGGCCA | |
| | | | | | TGGGTCTGGCCGCCCACACCGACTCCACCCTTCTCACAA | |
| | | | | | TCCTTTACCAAAACAACATAAGCGGGTTGC | |
| GS425 | Glyma.1 | GmHOS15 | 31 | Glycine max | TTCACGCATGCTGCTTTTACTTTTGGTTATGAAGCTGGTA | 117 |
| | 7G165900.1 | | | | TTAACAAATGCTCCATTGATGGAAATTTGGTGCCGTCAG | |
| | | | | | GTGCTCTTGTTACATTTATTCAGAAGGGTCTACAGTACTT | |
| | | | | | TGAGATGGAAGCCAACTTGAGTAATTGTGATGCAGACCT | |
| | | | | | GGAAGAAGATTTTTCATTCTTACAACCTTTGGATCTTATC | |
| | | | | | ACGAAAGATGTGCATGAACTTAGGCAAATGATAAGTGA | |
| | | | | | AAGAAGAAAAAAACTACAAAAGGAAAGAAAGAAGGAA | |
| | | | | | TTTGAAAAGGAGCATGAAGGTGAACGT | |
| GS439 | Glyma.0 | GmeIF_5A | 32 | Glycine max | GGAGCCTCCAAGACCTATCCTCAACAAGCCGGAACCAT | 118 |
| | 6G197300.1 | | | | CCGCAAAAACGGTTACATCGTCATCAAGGGTCGTCCCTG | |
| | | | | | CAAGGTTGTTGAAGTTTCTACTTCCAAGACTGGTAAGCA | |
| | | | | | CGGTCATGCCAAGTGTCACTTTGTTGGGATTGATATTTTC | |
| | | | | | ACTGCCAAGAAGCTTGAAGATATTGTGCCCTCTTCTCAC | |
| | | | | | AACTGTGATGTTCCCCATGTGAATCGTACTGATTACCAG | |
| | | | | | TTGATTGACATTGCTGAGGATGGATTTCTTAGCTTGCTTA | |
| | | | | | CTGAAAATGGGAACACCAAGGATGACCTGAAGCTTCCT | |
| | | | | | ACTGATGAGAGTTTGCTTACTCAGATAAAGGATGGGTTT | |
| | | | | | GCTGAGGGTAA | |
| GS2107 | AB181948.1 | GmCLH2 | 33 | Glycine max | ATGGCGCAGAGAGCTCAACCAGCGTTGGCCACCACGGA | 119 |
| | | | | | TGTTTTTCAGAAGGGAGATATTCATTGGAAGCAATTCAA | |
| | | | | | TGTTGAAACATCCACTGCTTCTTCCTCACCTCCAAAACC | |
| | | | | | ATTGCTTATTTTTACACCAACCGTGCCTGGCTTATACCCT | |
| | | | | | GTAATATTGTTTTGCCATGGCTTTTGTATTCGCACTAGCT | |
| | | | | | ACTACTCTAAGCTCCTAGCCCACATAGTTTCACATGGAT | |
| | | | | | TCATACTT | |
| GS2111 | Glyma | GmSPL121 | 34 | Glycine max | GCTCATGTGGTGGTTGAGATTCCAAGCAATACGACGGA | 120 |
| | 10G009200 | | | | GAGCAATACAAACGAGAAGCAAAATGAGTTATCCACTA | |
| | | | | | CCTTTGAGATTGGAAAGGCTGAAGTAATACGTGGCCAA | |
| | | | | | GGGCACTGTAAGCTTTGTGACAAGAGAATTTCTTGCAGA | |
| | | | | | ACTGCAGTAGGCAGGTCTTTGGTATACAGACCTGCAATG | |
| | | | | | CTCTCAATGGTGGCAATAGCCGCTGTCTGTGTCTGTGTG | |
| | | | | | GCCCTTCTGTTTAAAAGCTCCCCCGAAGTTATATGTATG | |
| | | | | | TTCCGACCCTTCAGGTG | |
| GS5363 | Lsat_1 | LsCPR5 | 35 | Lactuca | TTTGTTGCTCAGGTGTTGGAAAAGAAAGAAGCAACTGG | 121 |
| | v5_gn_2 | | | sativa | AGAAAAGATGTCTATAGATCATCTCTCCCAGATATGCAC | |
| | 65461.1 | | | | TTTGGCAGTTAAAGAATCATTGTCTCATGTTTTTGGCAAC | |
| | | | | | AAATTTGATTACTTTGTAAGCAACTTCGAAAGATCATTT | |
| | | | | | CAGAGCACCTTGATGACACTCAGAGTAATCAATGAAAC | |
| | | | | | ATCAGAAAACAGTAAAAGATCATATCAACACGTGGAAG | |
| | | | | | GGAGTTCAACCTATGATTTTCATTTTGATATGAAAGAAA | |
| | | | | | ACACAGCCACAAGCCAAGAACAAGCCACCATTGTTT | |
| GS5461 | Lsat 1 | LsCPR5 | 36 | Lactuca | CCGTCCTCGATGTTGTGGCTTTGTTGCTCAGGTGTTGGA | 122 |
| | v5_gn_2 | | | sativa | AAAGAAAGAAGCAACTGGAGAAAAGATGTCTATAGATC | |
| | 65461.1 | | | | ATCTCTCCCAGATATGCACTTTGGCAGTTAAAGAATCAT | |
| | | | | | TGTCTCATGTTTTTGGCAACAAATTTGATTACTTTGTAAG | |
| | | | | | CAACTTCGAAAGATCATTTCAGAGCACCTTGATGACACT | |
| | | | | | CAGAGTAATCAATGAAACATCAGAAAACAGTAAAAGAT | |
| | | | | | CATATCAACACGTGGAAGGGAGTTCAACCTATGATTTTC | |
| | | | | | ATTTTGATATGAAAGAAAACACAGCCACAAGCCAAGAA | |
| | | | | | CAAGCCACCATTGTTTGACCAGGATGGGCACCAC | |
| GS6396 | Niben 10 | NbEIN5 | 37 | Nicotiana | CAAGTGCAAATGCTCTTCCTGAAAAGTACAGGATGCTAA | 123 |
| | 1Scf035 | | | benthamiana | TGATGGATTCATCATCGCCAATTTCTGATTTTTACCCAAC | |
| | 65g06002.1 | | | | AGATTTTGAACTTGACATGAATGGAAAGCGTTTTGCATG | |
| | | | | | GCAGGCTGTTGTAAAATTGCCGTTCATCGACGAGAAGA | |
| | | | | | AGTTACTTGCTGAAACGAAAAAGCTTGAAGACACATTAT | |
| | | | | | CGGAAGATGAGCAACTCAGGAACAGTGTACTGTTCGAT | |
| | | | | | TTGCTCTATGTTCATCATCACCATACTTTGGCTCCATATA | |
| | | | | | TCATTTCGTACTATCATCACAATAATAAGGTGCCTCCAG | |
| | | | | | GGGAACAAAGGCCATGGCTAATTGATGCAAATCTGA | |
| GS6397 | Niben10 | NbEIN5 | 38 | Nicotiana | AATCTGCTGATGTGCATCTATAGAAGAGAGTTTACAACA | 124 |
| | 1Scf044 | | | benthamiana | ATGGGTGGTTATCTTACAGATGCGGGTGAGGTTTTTTTG | |
| | 87g01005.1 | | | | GATAGAGTAGAACATTTTATTCAGGCGGCAGCAGTTTAT | |
| | | | | | GAAGACCAAATCTTTCAAAAGAGAGCTAGGATAAAGCA | |
| | | | | | GTCAATAGAAAATAATGAAAGAGCAAGAAATGAGGTTA | |
| | | | | | ATGCTCAGCCACAGCCTCCGGCAGAGGACAAGGTGAAG | |
| | | | | | TTAGGGGAGCCTGGATACAAGGAAAGGTACTATTCCGA | |
| | | | | | GAAATTTGGTGTATCAACTCCAGAGGGTATTGATGAAGT | |
| | | | | | CAGACAAGATGTAGTCCAGAAGTATGTGGAAGGATTAT | |
| | | | | | GT | |
| GS6403 | Niben 10 | NbEIN5 | 39 | Nicotiana | CTCATTAGTCCGTCCAAGAAAACTCCTCTATATGGCTAT | 125 |
| | 1Scf035 | | | benthamiana | TGATGGAGTGGCTCCTAGAGCTAAGATGAACCAGCAGC | |
| | 65g0600 | | | | GAACCAGGCGTTTTAGAGCCGCTAAAGATGCCGCTGAA | |
| | 2; Niben | | | | GCTGAAGCTGAAGAAAAAAGGTTGAGGGAAGAGTTGTA | |
| | 101Scf0 | | | | ACCTTCCTGGCTTCGACCCAAATACCCGTCATTGTCTTTA | |
| | 4487g01005 | | | | TGGCCTAGATGCAGATCTGATTATGCTGTCTCTTGCTACT | |
| | | | | | CATGAAGTCCATTTTTCAATATTGAGGGAGGTGATTACA | |
| | | | | | TCACCAGGTCAGCAGGAGAAGTGTTTTG | |
| GS6411 | Solyc04 | SIEIN5 | 40 | Solanum | CTCCATTTGCTTCTGATCTCAAAGGTCTAGCAGATTTGG | 126 |
| | g049010.3.1 | | | lycopersicum | AAATTACTTTTTTCCCAGGGGAACCATTTAAGCCCTTCG | |
| | | | | | ATCAGCTAATGGGTGTTCTACCAGCTGCAAGTGCAAAAG | |
| | | | | | CTCTTCCTGAAAAATACAGGATGCTGATGATGGATCCTT | |
| | | | | | CATCACCAATTTCTGATTTTTACCCAACAGATTTCGAACT | |
| | | | | | TGACATGAATGGGAAGCGTTTTGCATGGCAGGCTGTTGT | |
| | | | | | AAAATTGCCGTTCATCGATGAGAAAAAGTTACTTGCTGA | |
| | | | | | AACAAAGAAGCTTGAAGACACGTTAACGGACACGGAGC | |
| | | | | | AACTAAGGAACAGTGTAATGTTCGATTTGCTCTAT | |
| GS5365 | Solyc04 | SICPR5 | 41 | Solanum | CAACAGTTGGCTTCTGCTTCTTCAAGCATGATTTTATCCA | 127 |
| | g054170.5.1 | | | lycopersicum | ATACAGGGATTAGCCAATCTGTGCATAGGACTTTGGAAA | |
| | | | | | GATCTCTCACAGAACAAGCTAGGTCGAATGACCTTAAG | |
| | | | | | ACATTTGAGATTGGTCTTACAATGAGGAAGTTGCAACTT | |
| | | | | | AAAGAAAGGCAGCTGGCTCTCAGTTCTGATGCTAACCTT | |
| | | | | | TTGGAGAGGGTCAAACTATCATTTGGTTTCTCTAAAACG | |
| | | | | | TCCTTCAAAACTGAAAAGTTCAAAAATCAAGTAGAAGA | |
| | | | | | CTCAAAACATGCTGAGCTACTTAAAACCTGTATAGA | |
| GS5463 | Solyc04 | SICPR5 | 42 | Solanum | CCGTCCTCGATGTTGTGGCCAACAGTTGGCTTCTGCTTCT | 128 |
| | g054170.5.1 | | | lycopersicum | TCAAGCATGATTTTATCCAATACAGGGATTAGCCAATCT | |
| | | | | | GTGCATAGGACTTTGGAAAGATCTCTCACAGAACAAGCT | |
| | | | | | AGGTCGAATGACCTTAAGACATTTGAGATTGGTCTTACA | |
| | | | | | ATGAGGAAGTTGCAACTTAAAGAAAGGCAGCTGGCTCT | |
| | | | | | CAGTTCTGATGCTAACCTTTTGGAGAGGGTCAAACTATC | |
| | | | | | ATTTGGTTTCTCTAAAACGTCCTTCAAAACTGAAAAGTT | |
| | | | | | CAAAAATCAAGTAGAAGACTCAAAACATGCTGAGCTAC | |
| | | | | | TTAAAACCTGTATAGAGACCAGGATGGGCACCAC | |
| GS6263 | Spov3_c | SoWRKY33 | 43 | Spinacia | CAACCAGCACAACCCCAACAACAATCATGGAATTTCGA | 129 |
| | hr2.02059 | | | oleracea | TCAAAAGAATGATGGATCTTCGAACTCAATTACAGCTAT | |
| | | | | | GGTGAAATCAGAGTTTCCTGGTGAGAATTACAACCAAG | |
| | | | | | GAAGCGTTGTAAAGTATGAGACAGACCAAAACCCTCCC | |
| | | | | | TACCAACCAGCTCAATACATAAGAGAACAGAGAAGATC | |
| | | | | | AGAAGATGGGTATAATTGGAGAAAATATGGACAAAAAC | |
| | | | | | AGGTAAAAGGGAGTGAAAATCCAAGAAGTTATTACAAA | |
| | | | | | TGCACGTTTCCCAATTGTCCAATGAAAAAGAAGATTGAG | |
| | | | | | ACAGCAATAGATGGTCAGATTA | |
| GS6264 | Spov3_c | SoWRKY33 | 44 | Spinacia | CTTTGGTGGTGAGGGATTCAATTGGACTCAACTAGGTGC | 130 |
| | hr4.00824 | | | oleracea | TAATAAGGATGAAGGAAGAAACAACAATTTTACTTTCC | |
| | | | | | ATGCTTCTTCAAACAACTCAATTGAAGGGTGGAATTTGG | |
| | | | | | ATGAACCGAAGAAGCAAAATGAGATGATTAACAACAAA | |
| | | | | | GATCAAATCAATTCAAAATCCGAGTTTACTACTGTTAAT | |
| | | | | | CAAACCATGCCATATGAAATGAACACAACAAAAACAGA | |
| | | | | | TGTTGGTACTAATATGCAAAGAGGGTACAATGCTCAACC | |
| | | | | | AGTTCAGATGAATCGAGCACAGAAAAGAGCTGAAGATG | |
| | | | | | GGTATTGTTGGAGGAAATAT | |
| GS6265 | Spov3_c | SoWRKY11 | 45 | Spinacia | GACAGTCGCACAACCACCCGATGACTTTGGATTTCACAA | 131 |
| | hr4.02159 | | | oleracea | AACCGAACCCGAATACAAAACCATGTGCGTCGATTTCG | |
| | | | | | AATTCGGCGTCCTTTAGTATATCTTCATCGGCGAACTCA | |
| | | | | | TCGTCGTTTTTGTCTACTATTACCGGAGAAGGAAGCGTG | |
| | | | | | TCTAACAGAAAGCTATTCGGTGGATCTGTTTTTGTTCCA | |
| | | | | | CCTCCTACATCGTCGTCTGGGTTTGCTGCTTCTGGGAAA | |
| | | | | | CCTCCCCTTTCAACGTCTTCGTCTTCTCATCATAATAAGA | |
| | | | | | GTTGTCACCACCACCACCAAGAGTTTTCCGAAGACTTTT | |
| | | | | | CCGGTAAAATCT | |
| GS6266 | Spov3_c | SoHOS15 | 46 | Spinacia | AAAGAGAAAGAGGAAAAAGCTGGAAAAGTAAAGGATG | 132 |
| | hr3.00650 | | | oleracea | AAAAATCTGGGAAAGAGAGGGAAGAAAAAGCAGTGAA | |
| | | | | | AGAGAAGGAAGAAAATGTTGGGAAAGATAGGGAAGAG | |
| | | | | | AAATCTATCAAAGAGGAGGAGGAAAAACCCAAGTTACA | |
| | | | | | AGATGTGCCTTCTGATATGGAAACAGTTGTTGAGCGAGA | |
| | | | | | GGAGAAGCCTGCCATCAAATATGAAGAGAATAGAGTTT | |
| | | | | | GTCCTGATCCTGAGCCCATGGATATCTCTCCATCATCAC | |
| | | | | | CTTCCATGGAGTGTGAAATTTCTACTTCAGATGTGACAA | |
| | | | | | TATTAGAGG | |
| GS6267 | Spov3_c | SoBZR1 | 47 | Spinacia | GGAGGAACTTTACCCCGCCTACCATACCTGAATGTGATG | 133 |
| | hr3.03117 | | | oleracea | AATCTGACTGCTCTACTATTGATTCCGGACAGTGGATTA | |
| | | | | | GCTTCCAAAGGTTTTCCAGACCTCCAATTCCAAATTCCC | |
| | | | | | CCACATTTAACTTGGTTAGGCCAATGGTTCAGAAAATGT | |
| | | | | | CCTTGAATGATGCAATTCAGGATAGAGCAAGAGTTGCTG | |
| | | | | | AAGCTGAGAGTATGCAGGTTACCCCTTGGGAGGGGGAA | |
| | | | | | AGGATTCATGATGTAGGCATGGATGATCTTGAACTCACT | |
| | | | | | CTTG | |
| GS6268 | Spov3_c | SoEDR1 | 48 | Spinacia | CTATAATCAAGGTAATAATGATGAATCAAAGAATCTTTT | 134 |
| | hr1.01547 | | | oleracea | TGCGGAACTTAATCCATTCCATTTACGAGTTTCTGGCCA | |
| | | | | | AGCTTCACAGCAAAATAAACCCAAATACGAGATTGATG | |
| | | | | | AGTCTCAGAGATTTAAGAATGATGCTGTCTCCAGAAAAC | |
| | | | | | CGCCTTCTCCCATGATGTGGAAAAACCACCAGGCTATCA | |
| | | | | | ATTATGTCCCCAGGAATAAAGATTATGATATTGTTGAAA | |
| | | | | | GTCAACTCCCGAGAAATAACAATGGAAACAAAGATTCC | |
| | | | | | AGAGCGGCACCAGTAACTTCATCTAGAACATCAGCTTCC | |
| | | | | | CAAAATTTACGTCAAAAT | |
| GS6269 | Spov3_c | SoPAM16 | 49 | Spinacia | GCAGCTCAGGAAGCAATGAACAATACCGTACGCAGAGC | 135 |
| | hr3.02285 | | | oleracea | AAGTAAAGCCATCACAGAACAAGAAGCAAGGCAGATTC | |
| | | | | | TTGGCGTTCCTGAGAATGCGGCCTGGGAAGATATTTTGA | |
| | | | | | AAAAATATGATGTTTTGTTTGAGAGGAATGCCAAGCAAG | |
| | | | | | GGAGCTTCTACATCCAATCGAAGGTCCATAGAGCAAAG | |
| | | | | | GAGTGTCTAGAAGCGATACATCAAAGCAAAGGTCAAGG | |
| | | | | | TTCCGCCAATGGGTAA | |
| GS6270 | Spov3_c | SoPAM16 | 50 | Spinacia | GTTAATTCGGGCGGTTGGTCAAGCTTATCGTCAAGCTAT | 136 |
| | hr3.04494 | | | oleracea | TGTCAATGCCTCAAAAACTGGTGCTGCGCAAGAAGCAA | |
| | | | | | TGCAAAACACCATTCGTAGAGCAAGTCAGGCTCTTACAG | |
| | | | | | AACAAGAAGCAAGACAGATACTTGGAGTCCCCGAGAAA | |
| | | | | | GCCGCTTGGGAAGAAATTTTAAGAAAATATGATGTTTTA | |
| | | | | | TTTGAGAGGAATGCTAAACAAGGGAGCTTTTACCTCCAA | |
| | | | | | TCGAAGGTACATAGAGCAAAGGAACATCTGGAAGAAGT | |
| | | | | | GTATCGAAATAAAGGTTA | |
| GS6271 | Spov3_c | SoEXA1 | 51 | Spinacia | TATCAGGCCAGGTGGTAGAGAGAAACTCAAAAACAGCA | 137 |
| | hr3.03137 | | | oleracea | CAACCTACTTTGATTGTGGATTTGGGGATGAGTGTGCTA | |
| | | | | | AGTGCTGACTCTGTACCACCCAAGTTTTCTGAATCATCT | |
| | | | | | GTGAGCCCTAGGGAAAATCAGATTTTGATGTTTGAGAGA | |
| | | | | | ACAAATGATAATAAGCTTACACCAACTGGTACTCTAGAG | |
| | | | | | GAAAACTCTGAAAAGGAGTGTCATGAAGATGTATCTGC | |
| | | | | | AGCAAAAGACGCAAAAAATGTTGAATCACGTGAGGTTA | |
| | | | | | AGAAAACTTCCGACAAGAAATCAAGAAAGCTAAAGTCT | |
| | | | | | TCCAAGGCGCAACCA | |
| GS6272 | Spov3_c | SoWRKY40 | 52 | Spinacia | ATAATGGTGAGAGTAGTTCCAGTGATGAAGAGGATTCCT | 138 |
| | hr1.04969 | | | oleracea | ATGGAAAACCTAAAGAAGAGACTATTAAAGCTAAGATC | |
| | | | | | ACAAGGGTTGCTGTTCGTACTCAAGCTTCTGATTCCACC | |
| | | | | | CTTATTCTTAAGGATGGATACCAATGGAGAAAATATGGC | |
| | | | | | CAAAAAGTTACAAGAGATAACCCTTGCCCTAGAGCTTAC | |
| | | | | | TTCAAATGCTCCTTTGCTCCTGGTTGCCCCGTTAAGAAA | |
| | | | | | AAGGTACAAAGAAGTCTAGAAGACCAGTCAATGTTAGT | |
| | | | | | GGCAACGTATGAAGGAGAGCACAACCACCAACCACCAG | |
| | | | | | CACAACAAGAGGGTCAGG | |
| GS6273 | Spov3_c | SoWRKY4 | 53 | Spinacia | ATGACAAAACACAGGTGGACGAGAGAAAAAGTGGGAA | 139 |
| | hr4.03061 | 0 | | oleracea | TTTAGAAGAGGAGTTGAACAGGATGAACATAGAGAATA | |
| | | | | | AGAAGCTGACAAAAATGTTGGCATCCATGTGTAAGAAT | |
| | | | | | TACAGTATGCTGCAAAATCAGTTGATTGGTTTGATTAGT | |
| | | | | | CCGACGTCGAAAGGCGAAAATGAAGATCAGAATCCAAG | |
| | | | | | GAAGAGGAAGTTTATTGAAGCTGTGGAGTGTGGAATAC | |
| | | | | | AGGGGAACACAGAACAGAGTTGTATTAGCTACGATGAT | |
| | | | | | TCATTCAAAAGAGTAACTCTTCTTAGCAGCAAGCCTAAG | |
| | | | | | ATCACTAATAAAATGTTGATT | |
| GS6274 | Spov3_c | SoJAZ1 | 54 | Spinacia | GACTTGTAACCTTCTCAGCCAGTATGTTAAGGAGAAACG | 140 |
| | hr5.03089 | | | oleracea | CGCTCAAGGTGTTACTGGAAACATCGAGTCCCGAGGAC | |
| | | | | | CAACTTCAACGATGAATTTGTTCCCTGTGAGTGGGAAAC | |
| | | | | | ATGCAGCTGAAGATTCAAATGTTTGTGCTGGGAAAACA | |
| | | | | | ATCAATCTCTTTCCTCAGCTTTCTGGGTTTAATTCAAGCG | |
| | | | | | TGAACAAGCGTGCTAACCCCGAGCCTCCGACTCCCCAAA | |
| | | | | | TGACCATCTTCTATGGAGGGCAAGTCTTGGTCTTTAATG | |
| | | | | | ACTTGCCGACTGACAAGGCAAAAGAAGTCATGGACCTA | |
| | | | | | GCTTGTAGCTTTGAAG | |
| GS6275 | Spov3_c | SoJAZ7 | 55 | Spinacia | TATCACCATCTTCCACGTCGATCTCTTCAACAGCTTCTAC | 141 |
| | hr5.00565 | | | oleracea | GATCTCCATGGGATCTGTTAATGGAGATAATCCAAAAAA | |
| | | | | | GCAAGAACAACAAAGGACGATATTTTATAATGGAACTA | |
| | | | | | TCTGCGTTTGCGATGTTACCGATGTTCAGGCAAAAAGCA | |
| | | | | | TAATACAGTTAGCAAGTAGAAAAACGATACAAGAAATA | |
| | | | | | TGTACATCTACAACAATAACACCAACGTTACAATCTCAA | |
| | | | | | CAATTATGTACTCAAACGCAAAATGGTTTATCGGCCATG | |
| | | | | | AAGAAATCGTTACGCCAATTTCTCGAAAAGCGAAAGCA | |
| | | | | | TCGAATCCAAGCTACCT | |
| GS6276 | Spov3_c | SoPW220 | 56 | Spinacia | CTGGGGAAGATAGCATGGAGAAAGCAGAAGAAACTGCC | 142 |
| | hr4.04996 | | | oleracea | CCAATGACCATATTCTACAATGGGTCAGTTTCTGTTTAT | |
| | | | | | GATCTACCCAACGACAAGGCCGAAAGCATAATGAAGCT | |
| | | | | | TGCAATGGAAAAGAATGAATCAAAAAACGAAGGGAAG | |
| | | | | | GTGGTGGATTATTCATGTTTTAAGTCATCCCAAGCAACT | |
| | | | | | ACTAAGAATATCCTCGGACGACTTAATGATGATGATGGA | |
| | | | | | GATGATTCTGATATGCCTATTAAACGTAAGAAGTCATTG | |
| | | | | | CAGAGGTTCTTTGAGAAGCGCAAAGAGAGGTTGGTGTC | |
| | | | | | GCCATATACAAACATAGAACA | |
| GS6277 | Spov3_c | SoMPK6 | 57 | Spinacia | CTCCTCGAAGGGAGGCATTTAATGATGTATATATTGCAT | 143 |
| | hr3.01388 | | | oleracea | ATGAGCTGATGGACACTGATCTTCATCAAATTATCCGTT | |
| | | | | | CAAACCAAGGATTATCAGAAGAGCACTGTCAGTATTTTC | |
| | | | | | TCTACCAGATACTACGTGGATTGAAGTACATTCACTCTG | |
| | | | | | CAAATGTTCTACACAGGGATCTAAAGCCTAGCAATCTCT | |
| | | | | | TGCTTAATGCCAACTGTGATTTAAAAATCTGTGATTTTG | |
| | | | | | GACTAGCTCGAGTAACATCGGAGACTGATTTTATGACTG | |
| | | | | | AATATGTTGTGACTAGATGGTATCGGGCACCAGAGTTAT | |
| | | | | | TGTTGAATTCTTCA | |
| GS6278 | Spov3_c | SoSPL12I | 58 | Spinacia | AAGACGGTAGGAAAAAGGACTTTAGAATGGGATTTGAA | 144 |
| | hr3.03860 | | | oleracea | TGATTGGAAATGGGATGGTGATCTTTTTACTGCCAGTCC | |
| | | | | | ATTGAATTCTACACCTTTAGATTGTAGAAGTAAGCAGCT | |
| | | | | | TTTTCCTCTTGGACCAGAACTTGTGGCTAACACCAATGT | |
| | | | | | CTGTTCTAAAGAAATTAATAAAACTAATGAGAAAGGGA | |
| | | | | | AAAGGGAAATGGAGAAGAGAAGGAGGGGTGTGGTTGTT | |
| | | | | | CCCGAGGAGGAGTTGAATGATGAAGCTAGACCACTCAA | |
| | | | | | TTTGAAGTTGGGTGAACAGGTTTATCCTATAGCGGAAGG | |
| | | | | | TGAAGCTGATAAATTGGAAG | |
| GS6279 | Spov3_c | SoCEV1 | 59 | Spinacia | GAATGGTGGCTGGATCTTACAAAAGGAACGAACTTGTT | 145 |
| | hr2.00984 | | | oleracea | AGAATTCGCCATGATAGTACTGATAGTGGGTCCAAATCT | |
| | | | | | TTGAAGAATTTGGATGGACAAATCTGTCAGATCTGTGGG | |
| | | | | | GATACCGTGGGAGTTACTTCGAATGGTGGTGTCTTTGTC | |
| | | | | | GCTTGCAATGAATGTGCTTTTCCAGTTTGCAGGCCTTGTT | |
| | | | | | ATGAGTATGAGAGAAAAGATGGAAACCAGTGTTGCCCC | |
| | | | | | CAATGCAAGACAAGATACAAGAGACAGAAAGGGAGTCT | |
| | | | | | TCGAGTGCAGGGCGATGATGAAGAGGAGGATGTTGATG | |
| | | | | | ATTTGGATAATGAATTCAA | |
| GS6280 | Spov3_c | SoCEV1 | 60 | Spinacia | ATTTGAGAGGTTTAGATGGTATCCAAGGACCTGTCTATG | 146 |
| | hr3.03490 | | | oleracea | TGGGTACGGGATGTGTCTTCAACAGAACTGCTTTGTATG | |
| | | | | | GTTATGAGCCTCCTATCAAACCAAAGCCCAAGAAGAAA | |
| | | | | | GGTATCCTATCATCATGCTTTGGTGGATCTCGGAAGAAA | |
| | | | | | TCAAGCAAGAAAGACTCCAAGAAGAAATCAAAACATGC | |
| | | | | | TGACCCTACTGTGCCAATATTTAATCTGGAAGATATCGA | |
| | | | | | GGAAGGAGTTGAAGGTGCTGGATTTGACGATGAAAAGT | |
| | | | | | CACTACTAATGTCACAAATAAGCTTAGAGAAACGGTTTG | |
| | | | | | GTAAATCAGAAGTATTTGT | |
| GS6281 | Spov3_c | SoCEV1 | 61 | Spinacia | GAATGGTGGCTGGATCTTACAAAAGGAATGAACTTGTTA | 147 |
| | hr4.01946 | | | oleracea | GGATTCGCCATGATAGTACTGATAGTGGGTCCAAATCTT | |
| | | | | | TGAAGAATTTGGATGGACAAATCTGTCAGATCTGTGGGG | |
| | | | | | ATACAGTGGGAGTTACTTCGAATGGCGGTGTCTTTGTCG | |
| | | | | | CTTGCAATGAATGTGCTTTTCCAGTTTGCAGGCCTTGTTA | |
| | | | | | TGAGTATGAGAGAAAAGATGGAAACCAGTGTTGCCCCC | |
| | | | | | AGTGCAAGACAAGATACAAGAGACAGAAAGGGAGTCTT | |
| | | | | | CGCGTGGAGGGAGATGATGAAGAGGAAGATGTTGATGA | |
| | | | | | TTTGGATAATGAATTCAA | |
| GS6282 | Spov3_c | SoCPR5 | 62 | Spinacia | TAAGCTCACAAATTGTTGTTTCAAATATGGGAAATCACT | 148 |
| | hr3.02304 | | | oleracea | TGAGCAACTATTCAGTGCTCAGTACCATTGAGAAATCTG | |
| | | | | | TTGTTGAGCAGACGCGTGCAAATGACCTAAAGAGCGTT | |
| | | | | | GAAATAGGTCTAATTATGAAAAAGTTGCGGCTGAAGGA | |
| | | | | | AACTCAACTGGCTCTTAATTCTGATTTGAATTTGTTGGAG | |
| | | | | | AGATGTAAATTGTCTCTAGGAATTTCAAGAACATCCTTC | |
| | | | | | AAAGTCGAGAAGTTTAAGAATCAATTGGAAGAGACAAG | |
| | | | | | ACATAGTGAGCTCCTCAGAACATGTGCAGACTTTCTTGT | |
| | | | | | TGCTGATTTGCTTATAAT | |
| GS6283 | Spov3_c | SoCOI1 | 63 | Spinacia | CTCTTGCGTATCACTAGCTCTTGCAGAAACCTGAGAACA | 149 |
| | hr4.03202 | | | oleracea | CTGTTTCTGGAAGACAGTGGTGTCGAGGAGACTAATGGT | |
| | | | | | GAATGGTTTCACCAGCTGGCACTACACAATATAGTTCTT | |
| | | | | | GAAACTCTGAACTTCTATATGACTTTTATGGAGAAAATC | |
| | | | | | ACAGTTGAAGATCTTGAACTTCTTGCCAGAAATTGTCCT | |
| | | | | | TTAGTCTCTGTGAAAATTGGAGATACTGATCTTGTTCATC | |
| | | | | | TTGGAAAATTTCTTCGTACAGCGACGAGATTGCAAGAGT | |
| | | | | | TCTCCGGTGGTTCTTATAACGAAGATTTAGACCAATATG | |
| | | | | | GTAATATTGTATT | |
| GS6281 | Spov3_c | SoDMR6 | 64 | Spinacia | AACAACGCTTCATAACATCCAATCTTTCACTAATCTCTT | 150 |
| | hr1.04698 | | | oleracea | GTGGCCTACTAGGAACGATCATTTTTGTGAAACCATCCA | |
| | | | | | TGAATATGCAAAGACAGTATCGGAGCTAGAACAATTGG | |
| | | | | | TAATGAGGATGGTATTTGAGACTTACGGGGTGGAGAAA | |
| | | | | | TACCTTAACTCTCATCTCAACTCGACCACTTACCTTCTTC | |
| | | | | | GTATAGCAAAGTACAGGGTACCACTCAAGGATGAACTT | |
| | | | | | AATCTTGGTGCAGTTGGTCACACTGACAAAAACTTCATA | |
| | | | | | ACAATACTTCATCAGAATCAAGTTAATGGTTTGGAAGTT | |
| | | | | | CAAACTAAAGATGGTGAA | |
| GS6285 | Spov3_c | SoDHS | 65 | Spinacia | GCTCGAAAGGATTGAATCGCATTGGTAACTTATTGGTCC | 151 |
| | hr5.03721 | | | oleracea | CAAATGACAACTACTGCAAATTTGAGGATTGGATTGTTC | |
| | | | | | CAATTTTTGATAAAATGCTGGAAGAGCAAAATACAGAG | |
| | | | | | AAGATAACATGGACACCCTCAAAGTTGATCGCTCGTTTG | |
| | | | | | GGAAAAGAAATAAATGACAAGAGTTCATACCTTTTTTGG | |
| | | | | | GCCTACAAGAATAATATTCCAGTATTCTGCCCAGGTTTA | |
| | | | | | ACAGATGGATCACTTGGAGACATGTTGTATTTTCATTCA | |
| | | | | | ATACGGAATCCCGGTTTAATTGTTGACGTAGTTCAAGAT | |
| | | | | | ATAAGAGCCATGAATG | |
| GS6286 | Spov3_c | SoDHS | 66 | Spinacia | CAGATGAGGTTACAATTCCGATCATATGATCCTATCCAA | 152 |
| | hr6.01752 | | | oleracea | CCTATCTCATTCGTTCTACTACAAAGGAATTTCAGGTGT | |
| | | | | | AGAAATATGGGTGATAGTGATATTTTGGCGTCGGTTCGC | |
| | | | | | TCTGTGGTGTTCAAGGAGTCAGAAACCCTAGAAGGGAA | |
| | | | | | ATGCATTAAGACTGAGAGTTATACCCAGCTTTTTAAATC | |
| | | | | | GTTTGTCTCCACCGGTTTCCAAGCATCGAATCTCGGAGA | |
| | | | | | AGCTATTGATGTTGTTAATGAAATGATTGACGACTGGAA | |
| | | | | | AATGAGATA | |
| GS6287 | Spov3_c | SomelF-5A | 67 | Spinacia | TCTAAGACTGGCAAGCACGGACATGCTAAATGTAACTTT | 153 |
| | hr2.02673 | | | oleracea | GTCGGTATCGACATCTTCACTGGGAAGAAGCTTGAGGAT | |
| | | | | | ATTGTTCCATCTTCTCACAATTGTGATGTTCCCCATGTCA | |
| | | | | | CCCGTACTGATTACCAGCTCCTTGACATTACTGAGGATG | |
| | | | | | GTTTTGTTAGCCTGCTGACTGATAATGGAGATACTAAGG | |
| | | | | | ATGACTTGAAGCTGCCAACTGATGAAGTTCTGAATAAAC | |
| | | | | | AGATCAAGGATGGATTTGCTGAGGGGAAAGACCTGATT | |
| | | | | | GTGAGCGTGATGAGTGCCATGGGAGAAGAACAAATTAA | |
| | | | | | TGCCCTCAAGGACAT | |
| GS6288 | Spov3_c | SomelF-5A | 68 | Spinacia | AGCACGGTCATGCTAAGTGTAACTTTGTTGGTATTGACA | 154 |
| | hr2.02679 | | | oleracea | TCTTCAATGGGAAGAAGCTTGAAGATATTGTTCCATCTT | |
| | | | | | CTCACAATTGTGATGTTCCCCACGTCAATCGTACCGATT | |
| | | | | | ATCAGCTCATTGACATAGCTGAGGATGGTTTTGTTAGCC | |
| | | | | | TGCTGACTGAGAGTGGAGGTACAAAGGATGACTTGAAG | |
| | | | | | CTGCCAACTGATGAAACTCTTCTTAAGCAGCTCAAGGAA | |
| | | | | | GGATTTGCGGATGGAAAAGACCTGATTGTGAGTGTTCAG | |
| | | | | | AATGCCATGGGAGAAGAACAAATTTGTGGCGTCAAGGA | |
| | | | | | TATTGGTGGTGGATCTAA | |
| GS6289 | Spov3_c | SomelF-5A | 69 | Spinacia | CTTTCCCTCAGCAAGCTGGTACTATCCGTAAGAACGGTT | 155 |
| | hr2.02680 | | | oleracea | ACATCGTCATCAAGAATCGCCCCTGCAAGGTTGTTGAAG | |
| | | | | | TTTCCACTTCTAAGACCGGCAAGCACGGCCATGCCAAAT | |
| | | | | | GTAACTTTGTTGGTATTGATATCTTCAATGGGAAGAAGC | |
| | | | | | TCGAGGATATTGTTCCATCTTCTCACAATTCTGATGTTCC | |
| | | | | | CCATGTCACTCGTACTGATTACCAGCTCCTTGACATAAC | |
| | | | | | TGAGGATGGCTTTGTTAGCCTGCTGACTGACAATGGAGA | |
| | | | | | CACTAAGGATGACTTGAGGCTGCCAACTGATGATGCTCT | |
| | | | | | GCTGAAACAGCTTAA | |
| GS6290 | Spov3_S | SoETR1 | 70 | Spinacia | AAAGCAGTAATCTTCTTGCGACTCTGATAAATGATGTTT | 156 |
| | 01.00038 | | | oleracea | TGGACCTTTCACGGCTTGAAGATGGAAGTTTGAAGCTTG | |
| | | | | | ATAATGGAGTATTTAATCTCCACACGATGTTTAGAGAGG | |
| | | | | | TCCTTAATTTGATCAAGCCCATAGCATCTATCAAAAAGT | |
| | | | | | TGTTGATAACGACGAATTTGGCAACGGATGTGCCAGAGT | |
| | | | | | TTGCTGTTGGTGATGAAAAACGTCTAATGCAGGTTCTAT | |
| | | | | | TGAATGTTATTGGTAATGCTGTGAAGTTTACGAAAGAAG | |
| | | | | | GAAGTGTCTCAGTTACTGCTTTAGTTGCAAAAGCAGAAT | |
| | | | | | CTTTAAGAGATGCTC | |
| GS6291 | Spov3_c | SoEIN4 | 71 | Spinacia | CGGCTACTGTCGAGGAGTACATTAAAGTAAAGTTTGAGT | 157 |
| | hr5.02367 | | | oleracea | TTGACATAAGTGGAGGTTATCTGACAGACACTATTAGGA | |
| | | | | | GAACCCAGAATGATTCAAACAAGGAAGTATTGAGCTTC | |
| | | | | | AGCATGTGCAAAAGGCTTGCTCAGATGATGCAGGGTGA | |
| | | | | | TATATGGATATCTCTAAACTCAAATGGGATACCACAGAA | |
| | | | | | CATGACTCTTGTTCTACGATTTCAACAACAAAGATATAT | |
| | | | | | TCCTGAAGGAAACTTATATATGACAGAGCACACAGTTTC | |
| | | | | | TTCAAACTCTCGGTTCAGAGGGTTGAAAGTGTTGATTGC | |
| | | | | | TGATGATGATAATGTAAA | |
| GS6292 | Spov3_c | SoETR2 | 72 | Spinacia | TTGTGCTACTGCCATTACCCTTATCACCCTCATCCCTTTG | 158 |
| | hr3.05108 | | | oleracea | CTTCTCAAAGTCAAGGTCAGAGAATTCATGTTAAAGAAG | |
| | | | | | AAGACTTGGGATTTGGGTAGGGAGGTTGGGATGATTAA | |
| | | | | | GAAACAAAGTGAAGCTGGATGGCATGTTAGGATGCTTA | |
| | | | | | CTCATGAGATTCGCAAGTCCTTAGATAGGCATACAATCT | |
| | | | | | TGCACACAACTATGGTTGAGCTCTCTAAAACCTTGGATT | |
| | | | | | TGCTTACTTGTGCTGTTTGGATGCCCAATGAGAATAGGA | |
| | | | | | CTGAGATCATTTTGACTCATGGTTTAAAAGGAAGGAATT | |
| | | | | | TTCAGAGTTTCAGCAAC | |
| GS6293 | Spov3_c | SoCTR1 | 73 | Spinacia | TTCTACAGGTGCAGTTGTGAATGAGCAAGATGCAGGAG | 159 |
| | hr6.02024 | | | oleracea | ATCCTATGTATCTTAAGCAACTTGGTAGAAAGCTGCCCG | |
| | | | | | ACGTAAACAATCCTATACCAAATGACCAAGTACATTCTC | |
| | | | | | AGTTTGTTGGTCATGATAGAGAACCCCAGTGTTCTGAAT | |
| | | | | | CTAGTAATCCTCCACAAAATATTGGAAGAGCATCTAATG | |
| | | | | | CCATCAGAGAGTTTCTTCCATCAAGATTTGTTCCTCCTAG | |
| | | | | | CAGACGAGGGACCCAGGCATTAACTGATTCAGCGAGAA | |
| | | | | | ATGCATCAAAAGATTTTCGGTTTGAGGCTAGTCAACTAG | |
| | | | | | TTTCAAGTAGAAGTGAA | |
| GS6294 | Spov3_c | SoMPK4a | 74 | Spinacia | CTGGCCTTGTTTGTGCTGCTGTTAATTCAGAAACACACC | 160 |
| | hr2.00919 | | | oleracea | ATGAAATTGCTATCAAGAAGATTGGTAATGCATTCGACA | |
| | | | | | ATATAGTAGATGCAAAAAGGACACTGAGAGAGATCAAA | |
| | | | | | CTACTTCGCCATATGGATCATGAAAATATTATTGCGATT | |
| | | | | | AAGGATATCATTCGACCACCAAAGAGAGAAGCATTCAA | |
| | | | | | TGATGTTTACATTGTTTATGAACTGATGGATACTGATCTT | |
| | | | | | CATCAGATAATTCGCTCTAACCAAGCTTTGACTGATGAT | |
| | | | | | CATTGCCAGTACTTCTTATATCAGCTCTTACGTGGCTTAA | |
| | | | | | AATATGTACACTCT | |
| GS6295 | Spov3_c | SoMPK4a | 75 | Spinacia | TACGGCATTGTTTGCTGTGCACGAAATTCAGAGACACAA | 161 |
| | hr3.01852 | | | oleracea | GAAGAGGTCGCCATTAAGAAGATTGCAGATGCTTTTAAC | |
| | | | | | AATAGAATTGATGCTAAAAGGACTTTGCGAGAAATCAA | |
| | | | | | ACTTCTGTGTCACATGGACCATGACAATGTTATCAAGAT | |
| | | | | | AAAGGACATCATACGACCACCTGAAAGAGAGAAGTTCA | |
| | | | | | ATGATGTCTACATTGTTTATGAATTAATGGATACTGATCT | |
| | | | | | TCATCAAATTACTCGCTCTAACCAGGAACTCACAGAAGA | |
| | | | | | TCATTGCCAGTACTTCTTATATCAAATCCTACGCGGGCT | |
| | | | | | AAAATATATACACTCG | |
| GS6296 | Spov3_c | SoMPK4a | 76 | Spinacia | TATGGTATTGTCTGTGCTGCTGTCAATTCAGAGACACGA | 162 |
| | hr5.00965 | | | oleracea | GAAGAAGTTGCAATCAAGAAGGTTAGCAATGCATTTCA | |
| | | | | | TAACAGAATTGATGCCAAAAGAATACTCCGCGAAATCA | |
| | | | | | AACTTCTTCGCCACATGAATCATGACAATGTTATTTCCA | |
| | | | | | TCAGGGACCTAATACGACCTCCTCAGAAAGAGAACTTC | |
| | | | | | AATGATGTATACATGGTTCATGAACTCATGGATACTGAT | |
| | | | | | CTTCACCAAATAATAAGATCCAACCAGCAGTTGACAGAT | |
| | | | | | GATCATTGCCGGTATTTTCTGTATCAAATATTACGAGGG | |
| | | | | | TTGAAGTACATTCACTCT | |
| GS6297 | Spov3_c | SoRPLK3 | 77 | Spinacia | ATGATGTGTTGGATTGCTTGAAGGAAGATAACATTATTG | 163 |
| | hr2.02651 | | | oleracea | GGAAAGGTGGTGCTGGGATTGTTTACAAAGGAAATATG | |
| | | | | | CCAAATGGTGATCAAGTCGCTGTTAAGCGTCTTCCTGTA | |
| | | | | | ATGAGTAGGGGTTCATCTCATGATCATGGGTTTAATGCT | |
| | | | | | GAGATTCAGACTTTAGGGAAGATTAGACATAGGCATATT | |
| | | | | | GTTAGATTGTTAGGGTTTTGCTCCAACCATGAGACTAAT | |
| | | | | | CTGTTAGTGTATGAGTACATGCCTAATGGAAGCTTAGGG | |
| | | | | | GAGGTGATACACGGTAAGAAAGGCGGTCATTTGCATTG | |
| | | | | | GGATACAAGGTATAAGAT | |
| GS6298 | Spov3_c | SoBZR1; So | 78 | Spinacia | CTCCAATTCCAAATTCCCCCACATTTAACTTGGTTAGGC | 164 |
| | hr3.0311 | WRKY40 | | oleracea | CAATGGTTCAGAAAATGTCCTTGAATGATGCAATTCAGG | |
| | 7; Spov3 | | | | ATAGAGCAAGAGTTGCTGAAGCTGAGAGTATGCAGGTT | |
| | chr1.04 | | | | ACATGGTGAGAGTAGTTCCAGTGATGAAGAGGATTCCT | |
| | 969; Spo | | | | ATGGAAAACCTAAAGAAGAGACTATTAAAGCTAAGATC | |
| | v3_chr4. | | | | ACAAGGGTTGCTGTTCGTACTCAAGCTTCTGATTCCACC | |
| | 03061 | | | | CTTATACAAAACACAGGTGGACGAGAGAAAAAGTGGGA | |
| | | | | | ATTTAGAAGAGGAGTTGAACAGGATGAACATAGAGAAT | |
| | | | | | AAGAAGCTGACAAAAATGTTGGCATCCATGTGTAAGAA | |
| | | | | | TTACAGTAT | |
| GS6299 | Spov3_c | SoJAZ_fam | 79 | Spinacia | TCAGACCCTGATTCCAACCCAGAAATCAAACCAGAACTT | 165 |
| | hr5.0308 | ily | | oleracea | GACTGTTGCTCCAAAATTTGTGAAGAACGTCATACCAGC | |
| | 9; Spov3 | | | | TACCGTGCCTAATGTTTCTGATCTGCCTATGTTACCGATG | |
| | chr5.00 | | | | TTCAGGCAAAAAGCATAATACAGTTAGCAAGTAGAAAA | |
| | 565; Spo | | | | ACGATACAAGAAATATGTACATCTACAACAATAACACC | |
| | v3_chr4. | | | | AACGTTACAATCTCAACAATTACTGTTTATGATCTACCC | |
| | 04996 | | | | AACGACAAGGCCGAAAGCATAATGAAGCTTGCAATGGA | |
| | | | | | AAAGAATGAATCAAAAAACGAAGGGAAGGTGGTGGATT | |
| | | | | | ATTCATGTTTTAAGT | |
| GS6300 | Spov3_c | SoDHS | 80 | Spinacia | TTGGATTGTTCCAATTTTTGATAAAATGCTGGAAGAGCA | 166 |
| | hr5.0372 | | | oleracea | AAATACAGAGAAGATAACATGGACACCCTCAAAGTTGA | |
| | 1; Spov3 | | | | TCGCTCGTTTGGGAAAAGAAATAAATGACAAGAGTTCA | |
| | chr6.01 | | | | TACCTTTTTTGGGCCTACAAGAATAATATTCCAGTATTCT | |
| | 752 | | | | GTTCAAGGAGTCAGAAACCCTAGAAGGGAAATGCATTA | |
| | | | | | AGACTGAGAGTTATACCCAGCTTTTTAAATCGTTTGTCT | |
| | | | | | CCACCGGTTTCCAAGCATCGAATCTCGGAGAAGCTATTG | |
| | | | | | ATGTTGTTAATGAAATGATTGACGACTGGAAAATGAGAT | |
| | | | | | A | |
| GS6301 | Spov3_c | SomelF5-A | 81 | Spinacia | CTGAGGATGGTTTTGTTAGCCTGCTGACTGATAATGGAG | 167 |
| | hr2.0267 | | | oleracea | ATACTAAGGATGACTTGAAGCTGCCAACTGATGAAGTTC | |
| | 3; Spov3 | | | | TGAATAAACAGATCAAGGATGGATTTGCTGAGGGGAAA | |
| | chr2.02 | | | | GACTGCCAACTGATGAAACTCTTCTTAAGCAGCTCAAGG | |
| | 679; Spo | | | | AAGGATTTGCGGATGGAAAAGACCTGATTGTGAGTGTTC | |
| | v3_chr2. | | | | AGAATGCCATGGGAGAAGAACAAATTTGTGGCGTCAAG | |
| | 02680 | | | | GATAAGCACGGCCATGCCAAATGTAACTTTGTTGGTATT | |
| | | | | | GATATCTTCAATGGGAAGAAGCTCGAGGATATTGTTCCA | |
| | | | | | TCTTCTCACAATTCTGATGTTCCCCATGTCACTCGTACTG | |
| | | | | | ATTA | |
| GS6302 | Spov3_c | SoMPK4a | 82 | Spinacia | ATCAAGAAGATTGGTAATGCATTCGACAATATAGTAGAT | 168 |
| | hr2.0091 | | | oleracea | GCAAAAAGGACACTGAGAGAGATCAAACTACTTCGCCA | |
| | 9; Spov3 | | | | TATGGATCATGAAAATATTATTGCGATTAAGCAAACTTC | |
| | chr3.01 | | | | TGTGTCACATGGACCATGACAATGTTATCAAGATAAAGG | |
| | 852; Spo | | | | ACATCATACGACCACCTGAAAGAGAGAAGTTCAATGAT | |
| | v3_chr5. | | | | GTCTACATTGTTTATGAATTAATGTCTGTGCTGCTGTCAA | |
| | 00965 | | | | TTCAGAGACACGAGAAGAAGTTGCAATCAAGAAGGTTA | |
| | | | | | GCAATGCATTTCATAACAGAATTGATGCCAAAAGAATA | |
| | | | | | CTCCGCGAAATCAAA | |
| GS6303 | Spov3_S | SoETR1; So | 83 | Spinacia | CCAGAGTTTGCTGTTGGTGATGAAAAACGTCTAATGCAG | 169 |
| | 01.0003 | EIN4; SoET | | oleracea | GTTCTATTGAATGTTATTGGTAATGCTGTGAAGTTTACG | |
| | 8; Spov3 | R2 | | | AAAGAAGGAAGTGTCTCAGTTACTGCTTTACGGCTACTG | |
| | chr5.02 | | | | TCGAGGAGTACATTAAAGTAAAGTTTGAGTTTGACATAA | |
| | 367; Spo | | | | GTGGAGGTTATCTGACAGACACTATTAGGAGAACCCAG | |
| | v3_chr3. | | | | AATGATTCAAACAAGGAAGTATTTGGGGTTGCTTTCAGT | |
| | 05108 | | | | TCTAAAAGATGAGAATTTAAGCAGTGAACAGAAACTTA | |
| | | | | | TTGTTGACACAATGATGAAGACAGGGAATGTCCTTTCTC | |
| | | | | | TTTTGATCAATGAT | |
| GS6304 | Spov3_c | SoCEV1 | 84 | Spinacia | ATTTGAGAGGTTTAGATGGTATCCAAGGACCTGTCTATG | 170 |
| | hr3.0349 | | | oleracea | TGGGTACGGGATGTGTCTTCAACAGAACTGCTTTGTATG | |
| | 0; Spov3 | | | | GTTATGAGCCTCCTATCAAACCAAAGCCCAAGAAGAAA | |
| | chr2.00 | | | | GGTATCCTATCATCATGCTTTGGTGGATCTCGGAGAAAG | |
| | 984; Spo | | | | AAGCCATTCATGTTATTAGCTGTGGATACGAGGACAAGT | |
| | v3_chr4. | | | | CTGAATGGGGTAAAGAGATTGGGTGGATATATGGTTCA | |
| | 01946 | | | | GTGACAGAAGATATTTTGACTGGATTTAAGATGCATGCT | |
| | | | | | CGTGGGTGGATGTCGATCTACTGCATGCC | |
| GS5364 | SOV3g0 | SoCPR5 | 85 | Spinacia | AACCAATGTATTTGGCGACAAGTTTGATGCTTTTGCAAG | 171 |
| | 30270.1 | | | oleracea | AAACTTCGAGAAATCTTTTGGAAGTACCCTCAGCACTCT | |
| | | | | | GACTTTGATAAATAACTCATCTATAGACAATAAAGATGA | |
| | | | | | TCACGTTAGGAATAACATTGTTGGCGATTGTATTTCTGA | |
| | | | | | TGCAACCCAATCCATCTCCTTTAACAAAGGAGATTGTTC | |
| | | | | | CGCAAGAAGTAGTGATGTTATAGAGGATTGCAGAAGAT | |
| | | | | | CAATAAATGGCGCTACTGGGCCTCAGCGTCGTATAAAA | |
| | | | | | ATGGTTGAAGAGGTAGAAGATATTCAGTCAGCAGACT | |
| GS5462 | SOV3g0 | SoCPR5 | 86 | Spinacia | CCGTCCTCGATGTTGTGGCAACCAATGTATTTGGCGACA | 172 |
| | 30270.1 | | | oleracea | AGTTTGATGCTTTTGCAAGAAACTTCGAGAAATCTTTTG | |
| | | | | | GAAGTACCCTCAGCACTCTGACTTTGATAAATAACTCAT | |
| | | | | | CTATAGACAATAAAGATGATCACGTTAGGAATAACATT | |
| | | | | | GTTGGCGATTGTATTTCTGATGCAACCCAATCCATCTCCT | |
| | | | | | TTAACAAAGGAGATTGTTCCGCAAGAAGTAGTGATGTTA | |
| | | | | | TAGAGGATTGCAGAAGATCAATAAATGGCGCTACTGGG | |
| | | | | | CCTCAGCGTCGTATAAAAATGGTTGAAGAGGTAGAAGA | |
| | | | | | TATTCAGTCAGCAGACTGACCAGGATGGGCACCAC | |
| GS6562 | Lsat_1 | LsHOS15 | 265 | Lactuca | TTACAAAAAGGCCCCTTAGATGTTGCTGTTTTGAAACAC | 275 |
| | v5_gn_1 | | | sativa | TACAGAGGTAGAACAAATGATAAGAGCAAAGATGTAAC | |
| | 24041.1 | | | | CACACTTGACTGGAATGGTGATGGAACTTTACTTGCAAC | |
| | | | | | AGGTTCTTATGATGGACAAGCCAGAATATGGAGTAAAG | |
| | | | | | ATGGGGAGCTGATGAGTACATTAACAAAACATAAGGGG | |
| | | | | | CCAATATTCTCTTTAAAATGGAACAAGAAAGGAGACTAT | |
| | | | | | CTTTTAAGTGGCAGTGTAGACAAAACAGCAATTGTATGG | |
| | | | | | GACATTAAGACTGGAGAATGGAAACAACAGTTTGAGTT | |
| | | | | | TCATGCAGCT | |
| GS6563 | Lsat 1 | LsEDR1 | 266 | Lactuca | ACTGGTGTTGAGGATGATGCAGTCAACATTATCAAGTTG | 276 |
| | v5_gn_8 | | | sativa | GATAATGAAAGTGAGTTTCTAGTGGATCTAATGGCAGCC | |
| | 116101.1 | | | | CCTGGAACACTTATACCCGCGGATATTTTCAGTGGAAAA | |
| | | | | | GATTCCTCCTTGAAACCAAACATCCCAAAATCAAGTCAA | |
| | | | | | CTTCCAGGTGTCCAGTCAACTAAAGATTTCTCACTTCCTT | |
| | | | | | CAAGACCCGTTTCAAACAATAATATCAATGGTGCCAGTA | |
| | | | | | GTCAAAACATTGAAGTGCAAAACAACACTAGTCAATTA | |
| | | | | | GATCACATCCCTTCATCAGCAATCGGAAATTCTCTTTAC | |
| | | | | | AAAGGA | |
| GS6564 | Lsat_1 | LsPAM16-1 | 267 | Lactuca | TCTGGAATAATGGCTAGGGCTCTTGTTCAAGCATATAGA | 277 |
| | v5_gn_1 | | | sativa | CAAGCCCTCCAAAATGCATCAAAATCAGGTGTTGCTCAA | |
| | 71380.1 | | | | GACACATTACAAAATGTTGTGCGTAAAGGAAGCAAAGC | |
| | | | | | TATGACTGAACAAGAAGCCAGACAAATTCTTGGTGTCA | |
| | | | | | ATGAACAATCATCATGGGAAGAAATCGCACAAAAATAT | |
| | | | | | GACAATTTATTTGAAAGGAACGCCAAGAATGGGAGTTTT | |
| | | | | | TATCTACAGTCAAAAGTTCATAGGGCTAAAGAATGTTTA | |
| | | | | | GAAACAACTTATCAAGCAGGTGAATATGCAGGTACTC | |
| GS6565 | Lsat_1 | LsPAM16-2 | 268 | Lactuca | ATGGCTGCTAAGATTCTTGCAAATTTGATTGTAATGGGC | 278 |
| | v5_gn_6 | | | sativa | TCTGGAATCTTGATTAGGGGAATGGTTCAAGCATATCGT | |
| | 56540.1 | | | | CAGGCTCTCCAAAATGCCTCAAAGTCAGGGGTTGCTCAA | |
| | | | | | GAAACATTACAGAATGCTGTGCGAAGAGGAAGCAAAGC | |
| | | | | | TATGACTGAACAAGAAGCGAGACAAATTCTTGGTGTGA | |
| | | | | | CTGAACAATCATCTTGGGAGGAGATTGCACAGAAATAT | |
| | | | | | GATAATCTATTTGAGAGAAATGCGAAGAATGGGAGTTTT | |
| | | | | | TATTTGCAGTCAAA | |
| GS6566 | Lsat_1 | LsEXA1 | 269 | Lactuca | AAGGTCCAGAAACCATCTACTAATGAGTCCAGTGTTGTG | 279 |
| | v5_gn_4 | | | sativa | AAAGAAGTAAAGCCTGTTGAAACTGTGAAAAAAGCCAC | |
| | 117960.1 | | | | TGAAAAGAAGTCAAAGAAACAGAAGTCTTCAAAGTCAC | |
| | | | | | AATCGTCTGACATGGCTAAAACAGTATCTAAACCCCAAC | |
| | | | | | AGCAGCAGCCAAAGGAACCTGAAACTGATTTGACCAAA | |
| | | | | | GTCAACGAGGTCGAAATGGAAACATCTCAAGTAGAAGA | |
| | | | | | GACCAAATCTAGAGTAGCCATGGAAGAGCCTGTATCTAT | |
| | | | | | GTCTAGTAATGTTAACCAAATCCCTCAAGATAACAACAC | |
| | | | | | ACAACAAGTT | |
| GS6567 | Lsat_1 | LsMPK6 | 270 | Lactuca | GATGCGAAGAGGACACTGCGAGAAATCAAGCTTCTCCG | 280 |
| | v5_gn_1 | | | sativa | TCATATGGATCATGAAAACGTTGTTGCAATTAGAGATAT | |
| | 74260.1 | | | | AATCCCACCTCCAGAAAGAACATCCTTCAATGATGTCTA | |
| | | | | | CATTGCTTATGAGCTCATGGACACTGATCTCCACCAAAT | |
| | | | | | AATCCGTTCTAACCAATCTTTATCAGAGGAACATTGCCA | |
| | | | | | GTATTTTCTGTATCAAATCCTTCGTGGGTTAAAGTACATA | |
| | | | | | CACTCTGCAAATGTGTTACACAGGGATTTAAAGCCAAGC | |
| | | | | | AATCTTTTATTGAATGCAAATTGTGATTTAAAAATATGT | |
| | | | | | GATT | |
| GS6568 | Lsat 1 | LsWRKY33-1 | 271 | Lactuca | TACAATTGGAGAAAATACGGTCAAAAACAAGTCAAAGG | 281 |
| | v5_gn_9 | | | sativa | AAGCGAAAACCCTAGAAGTTATTACAAATGTACGTTCCC | |
| | 14580.1 | | | | TAATTGCCCAACAAAGAAGAAAGTTGAAAGAAACTTAG | |
| | | | | | AAGGACATATCACGGAAATCGTTTACAAAGGAAATTTA | |
| | | | | | TGCAGCCAGCAACAACAATTTGAACAATCAACCATCGC | |
| | | | | | AGAAAAAATCAGATGATGGGTATAATTGGAGAAAATAC | |
| | | | | | GGGCAAAAGCAGGTGAAAGGAAGTGAAAATCCTAGAA | |
| | | | | | GCTATTACAAGTGCACGTATCCAAACTGTTCGAT | |
| GS6569 | Lsat 1 | LsBZR1-1 | 273 | Lactuca | ATCTAATCTGGGAATCATTCACCAAACAGTCAATTCCCT | 282 |
| | v5_gn_5 | | | sativa | CTTTCAATCTCCCCTACTTTGCATCTTCTGCTCCCACAAG | |
| | 29981.1, | | | | CCCAACTAGATATCAACGTTTAAAAGGGTAAAGGGGTG | |
| | Lsat_1_ | | | | AAGGCATGGAAAGGCGAGAGGTTTCATGAAGTCGGTTT | |
| | v5_gn | | | | TGATGATCTTGAGCTCACCCTTGGAAGTGGGAATGCTCA | |
| | 4 12228 | | | | AATATATTGAGAATGGAGGAGTGAAGGCATGGGAAGGC | |
| | 0.1, Lsat | | | | GAGAGGATTCATGATGTTGGAGTGGATGATCTTGAACTC | |
| | 1_v5_g | | | | ACTCTTGGAAGTGGGAATTCTAAAATATA | |
| | n_1_182 | | | | | |
| | 00.1 | | | | | |

*Each thymidine (T) within a nucleic acid sequence (dsRNA sequence) provided in Table 1 can be replaced with a uracil.

TABLE 2
Reverse Complement Sequences

| dsRNA | | SEQ |
|---|---|---|
| Name | Reverse Complement Sequence | ID NO |
| GS6434 | ATTGCTAGGTTTCAAATCTCGATGCAATACATTAGCTGAATGAACATATTTGAGCCCACGCAACAACTGAT | 173 |
| | ACAGAAAAAACCGACAATGATCATCAGTTAAGGGTTGGTTAGAGCGAATAATTTGGTGGAGATCAGTGTC | |
| | CATAAGCTCATAAACAATGTAAACATCATTGAAGTTCTCTCTCTGCGGTGGTTTTATTATATCCTTTACAGC | |
| | AATCACATTTTCATGATCCATATGCTTGAGAAGCTTAATCTCTCTCAAAGTCCTTTTAGCATCGATAATATT | |
| | GTCAAAAGCATTACCAATCTTCTTGATAGCTACCTCCTCTCCA | |
| GS6435 | AATCCATGATTCTTGTGACCATCTCTTCTTTTCTTGTATCTTCTAATTCGGTTTTGAATTTCTCCGCTCTGAAT | 174 |
| | GCAGCTTTCGAAACATCCAACTCTAGCGCCGCTTTCCCCAGGTTGTTTGATTCAAATTTTAATCCTAGCGCC | |
| | GTCTCTTTGCACCTTAACTCTCTTATTTGAAGACCAATCTCCACGGTCTTTAGGTCGTTTGCTCGTGTTTGCT | |
| | CTTTTAAAAACCTTTCGTCTGTGGTCAAAGCCATTGCTGAACTTCTAGGAGGGACACAAGAGAGTTGTAGA | |
| | GTCTCTTCGAAAAGGGCAAGCTCATTCATCAA | |
| GS200 | GAACATAATCTTTGCCAGGAAAGAGCGGTTGTCTTGTTATGATTTCACCAAGTATGCAACCAACAGACCAT | 175 |
| | ATATCAATGGCTGCTGTATATTCTGAACAATTAAGAAGCAATTCGGGAGCTCGGTACCATCTAGTGACCAC | |
| | ATACTCAGTCATAAAGTCAGTTTCAGATGTGGTTCTAGCAAGACCAAAGTCCGCAATCTTAAGGTCACAAT | |
| | TGGCATTCAATAGCAATTACTAGGCTTTAGATCTCGGTGCAAAACATTTGCTGAATGTACATATTTGAGCCC | |
| | TCGTAACAATTGATACAG | |
| GS201 | TACACTGACTGATCTCAGGCACAATAGGACCCGAGAACTTGTTGCCACTAAAATCAATCTTAGAAAGCTGT | 176 |
| | TGCAACCTCCCAATCTGGGGAGGTATCCGACCCGTGAACATGTTGCCATCAAGAATGAGCTTCTGCACGCT | |
| | GGAGAAGTTACCAATGGAGGGAGGTAGAACCCCAGAAAGCTGGTTGTTAGAGAGAGTAATCTGACCAAGA | |
| | TTAACAGCAACAGAACCCACCTCAGGAAACTCTCCAGAGAGATAAT | |
| GS275 | TAAGAAATGCAGCAAGCACATAATCTCCCAGTATAAGAGCCACACAATACCACTCCCACCCAATGTCTCTA | 177 |
| | CACATAGCTTGCCACAATAGCCACAAAAAATTCCCAACATTAAAAGGATGAAAGTGACTGGCATAGTCTG | |
| | TGATGTTGTGGTCGACCGCTGCAAGAGCAAATATGCAACAGCAAAAATCATCAAGATGCCAAATGCCATT | |
| | CGGCTCATAACTTGAACTTGACACCACAAAATATGCAACTTTGAATTGAATAAGACCATTGACTTGGGAGT | |
| | CCACCAGGAGGAAGATTCCT | |
| GS276 | TTTAACTTTTTGTGAGAGTTCATTTGGAAGCTCTTGGTCAATTGAAATCTGGTCAAGGTCTGATCTGGCCAC | 178 |
| | CCTATCAATGCATTGATTGACAAGTTGACTCAATTGACAATGGAAAGCAACAGTGAGGATTGGGATGACAT | |
| | CTTCCACAAGAGCCTTCCCTATAAAGTTAAGTAGACGTCTCTGGAAAAGTGATACAAACTCTGGTATTTGA | |
| | AAAATGTAAGAGGCATACATCAACTCCACAGCAAAGTTAATGGCAGGTCTACAAGCATCATGGGCACACA | |
| | CACTGTCAACACAAGTAGACACTTCCATTGGAGAGGGCTTGAGTTTACCAGTATATACATAGCCAAGGAAT | |
| | ATGGGGAAGGCTTCATATCCAACCTTGCCATAAGGCAACAAATCACTCATGTTATACTTCAATTTCCCTTCT | |
| | TTTTCTGATGAACCCTTCTCTCTCTTGAATAATTCATGGAAAAACTTACTCCTAGAGGCCAGAATACATCGA | |
| | TGAACACT | |
| GS277 | ACATCAATTCCTCTATCATCGGATGCATTGAGCTCCAAAATCATATTTTGGTACTGTGAACCATACAGTTTG | 179 |
| | CGTGCCACAGCAAGAATCGTTGATGTTTTGCCCGTGCCAGGAGGGCCATACAACAGAAGATGGGGCAATC | |
| | TGTTCTCAGTGGTTAGCCTATCAATTGTATCGACAATGTCACGGTGAGCAGCGACGTCGTCGAGGGACTGA | |
| | GGGCGGTATTTTTCGACCCACGGAATGGCCTTGCCGGCGGCGGGAGGGTTTCCGGCGAAGACGACACTTTT | |
| | GCCCTTGTCAGGACTGGCGGTGTAAGTGTGGTCGTCGATGTCCATGGGATGGGTCGTCTGCGCCAT | |
| GS278 | CAGTAGCAGGCTGAGCAAAGACTCCCTCTGCATGCTCCAGTTCAAGGTTGCATTGCGAGGTAAAAAATCAT | 180 |
| | CCTCAAAAACAACGACAAGGTACTGAAAGATTAACATGTTCCTCAAGGGCTCCATGCTTGCTGCTTCAAAT | |
| | TTGGTCTCTGCTAAAACTTCAGCCAGCCCTTCAATCAAGACTTGAAAGCTAGAGGGGTCAAGAGATCCACC | |
| | AGACTGTTTATCACCAGCTTCACTAACAGCAGTGGCCTTATCCACAAAATCGATAATGGGAGACCATGCCT | |
| | CTTTAACCACAACCAATTTTGATGGAGAGTTAGATGATGATCGGGAATCATCCGTGCCAGATAAATACACG | |
| | CGAGGGAAGTG | |
| GS279 | AACAACCCAGGCTTTTTGTGCTTGGGTTTAAGAGGAGGTTCATAACCATATAAAGCTGTCCTATTGAAGAC | 181 |
| | ACATCCAGTACCCACATAAACAGGACCTTGAATGCCATCCAAACCTCTCAAGTTTATATCAAAGAAAACTG | |
| | TATTACGATTGGCATATCGATCATTCCTATCAATACCATCAAACCTCTGTGGAAACTGGACATAGCAAACA | |
| | TGTTTACCAAGGTTTGGATCCATCATAAAGCACATAGCTTCCCTCAAGGCCTTGCTGTTGTTTATGTAGTGA | |
| | TCACAATCAAGATTCAATAAGAAAGGTCCATTGGTAAGGACTG | |
| GS280 | GAAAAGACCATCGGTGGTGAAACCGGAGCACTTGTCAAGCTTGAGAGAGTGAAGCACGTGGCCGCGGTCA | 182 |
| | CGAGCGAGATTCCGAAGATCGGAATCTTTGACAATCATACGGCGGAAGTGGAGACTCTTGAGGCAATCGA | |
| | AGTACTGAGAAATCTCCTTGACCCATGGGGTGACGTGGCCTCCCCAATCCTCGGGTATCAAGTTGAACATT | |
| | GCTGCTCGGGGCTTGCCCTTGAGCTTGAGCGACTCAAGGTGCGGGAAGCGGCGGCGGAGGCGCGCCGGCG | |
| | TGGTGGTGTAGCAGAGCGCGATGGTGACGTGCTTCCGAGTGAGGGAGTCGAGTTCGTACCAGCGTCGGCA | |
| | GACCTGTGAGACGGCGTCGCGGTCCTTGGGGTCGTCGATGTAAGGGATG | |
| GS282 | GATCCATTTGAGGCACCACTTCTATCAACACTTATTTGTTCATGATTATTTGATGTCTTTTCACCTATGCAAC | 183 |
| | CTCTCAATTGGCTATGATCTTTGAAGACTATGTCTAAAGAATCAAATATAGGGTCAGCAGGCTCATATAGA | |
| | GAAGCAAAAGCCCACAAAAGCTGAGCAAGTTCTTGTTCTTGGAAAGTGTGAATTATGTCTGAAGCCCTTTT | |
| | TGAAAACTCTGAGAAAAGATCAGGAGCAGAATGCTGCATTGCAGCAAAGGCACCTGCAATATTAGCAATG | |
| | TTTTGAGAGTTGAATTCCCCAACCTTGGTCAGTGCAACCTCTGCAATTCTATCCATTTCAGAAAGATAAAGC | |
| | AGCTCGCC | |
| GS283 | AGGCATACGATGGGTTCTGAAGGAAAACCTATGCTTCCTGTTCTAGATTTCACCATAGAAGATTTGAAGCC | 184 |
| | AGGGACCAATTCCTGGTTATCAACATGCACAAGTGTGCGCCAAGCATTTGAGGAGAATGGTTACTTTGTTG | |
| | CTGTGTATGATAAAGCTTCTATTGAGCTACAAAATGGTGTATTTGGCTCCATGAAGGAGCTGTTCGATCTCC | |
| | CAACAGAAACCAAACGAAGAAACATATTTGAGGGCATGCCCTTGAAAGGCTACGTTGGACAGCAC | |
| GS284 | AATCCTTCAAACGCTTCACATCAGATAACTTCACTGGCTTCACTATGAAATCCTCTGCACCCTCCTCCAAAC | 185 |
| | ATCTGTCTATGCGTGGCAAAACGTTTTCAGATGACATAATAACCACAGGCGTTTCCTTGAAAGTAGATGAT | |
| | TCCTTGATTCTCTTGAGTAATTCATAACCGGTCATTCCGGGCATACAATAGTCTGTGA | |
| GS285 | TGACCACAGATAAGCTTGCAATGGAAATGACAAATTTTACCATCTTGGAATGACCCGAATCCTCAAGCATC | 186 |
| | AAAGAAAAGGCAGAGCAAAATGCAACCATCATGGCCAAGATAGAACATATGAGTGTGAAAAGGCCAAAC | |
| | ATCAACTTGAGTGGCAAGGATTTAAGGAAATCTCTTTCAGCATAACGAGATGTGAGAATTCCAATAAAAGT | |
| | CATCACAGCAGTGGAGGAAGAAAAGAGTGAGATAGCATCAGCCACCATATAAGTTGTGAACATTCTTTTG | |
| | TGCAAAAACATAGGAATACCAGTATTTTGATCATTTCCTCCAGGAAGAGTGAAGGCTGCTGCAAACACAAT | |
| | GGTTATTATAAGAGTCCCCACTATTGTGAAGGATGTTGCCGTCTCCTTTGCCCATTTCTCTGCGTCCTTCAA | |
| | CAGTTCTTCATGTTTCTTA | |
| GS286 | CCACTGTCATCATCATCGCTATAGCAGTTTACACCTTGTTGAGACCTGATCTTCTCATAGACTTTGCACAAA | 187 |
| | ACCCACTTGCTCCAACTTTGATCATGTTTTCTTCTTCTCCTAGCACTTCGATATGAGGCAGTGTCAAACCCA | |
| | GAGGAACATATATAATATTCTTGCATTACCCAACTGGTTTCAGTACCTTGTGGAGCTTCACCTATGTGGAAC | |
| | ACCAAGTACTTCTTCATTCCCACTTTCTTATCAAAGGTTGACAATATAGGTTCCGTGACACCTGTGTCCTTC | |
| | CAATACCC | |
| GS287 | GCTCCAATAGCAATTTTCCGCCTAATGGCCCAGTTCAGCTTGATCCCAGCTTTCTTCTGATCATGTAGAACA | 188 |
| | TCCTCTAAACTTCCATATTTCATGTACTCATAAACCAAGAGCCTCTCTTCCCCTACCTTGCAGTATCCCAAC | |
| | AGGGGAACAAGGTTCCTGTGCTTGATCTTCCCAATGGTCTCCATTTCAGCAGTGAATTCCCTGTCCCCTTGG | |
| | CCGCTGACATGAATCAGCTTCTTGATAGCCACAACACTTCCATCCTTCAACTGAGCCTTGTAAACATCCCCA | |
| | AACCCACCAGAGCCAATGAGACTATCATTGTGAAAGCCATTGGTAGCGTCAAGAAGGTCCGCAAAAGTAA | |
| | GC | |
| GS288 | TCCCAACATCCAAAACTGGTGTCTTTCCGTACAAGTTCTTGAGCTCTAGAGGACCAAGTTTTGGACGACGA | 189 |
| | AGTCCAAATTGAGCTGTGTCTCCAAGCATGAGATGTGACATTAGAAGCAAAAATTGATCCACAAAACGTAT | |
| | GGGGAACCACTTGTTGGGAGTTTTCTTGTTCGCAGCTGTCAGACACTGCCTTTAAAACCTTGGCAGCTCTGA | |
| | GTTGAGAATCCTTCGAGACACCGATTGTACTTATCTCTATGAATTTAGTCATTTTCTTTGCTACCTCTTTATC | |
| | TTTGACAATAGCCCACCCTATGC | |
| GS289 | GGGTCAGAGATGGCTATGGAAATGGACGGTCGTAATTGAAACCGAAGAAGAAGAGTCATGCTTTGAGGAA | 190 |
| | AACCATGATTACAAGGTACTAACCATATGTTCCCCTGCATCAACTGGACTACCCTATTACTTGGAAAGTCCT | |
| | TGCCTCATCACCAATGACCAGATCAGGCAAAGACTTGTCAACATCAACTTCAAGACCAAAGCCTTTATATA | |
| | TACACAAACACTTGGCAGTGCAAGAAGCTTCCCTCAACCAGTTTCTTCTTGTGTGCGAATCAGGCCCATTTC | |
| | TCTATCAAGCTCTGCAGCCTTGTTCTTCAAAAATAATTCCCTAGTTTTAACACTTAATAAATCAGGAATAAT | |
| | ATGTACCAGCATGAGGGCAGTTGCACACGATACCACAGCAGT | |
| GS423 | GTGCCTAATGGGTTTGATGGATTGGTGATGAGCAAGCCCTTTACTCTTATATTATCCTCTTTGGCCTTCTCAT | 191 |
| | ACGCATCTTCCAATGCTTGCTTTGTCAACTTGAAATTGTTTGAGCTATCGCACATAACTGGAACAAGTTTAA | |
| | TTCCTGTTCTCCACCTCAAATCCCGGTCAAAACCTGGATAATAAGGAATGGGCACCAAAAATGCGTCACCA | |
| | GGGTCTGCCAAACAAAAGGTAGTGACTTCGTGTGCTCCAGTTGCTCCACCGCTCATGACAATACGGTCAGG | |
| | ATCAAACGTGACTCTGTTTCCTCTTGTTCTACCCATGAATTTAGCCACAGCATTTCTGAACTCGGGCAGACC | |
| | ATGATAAT | |
| GS436 | CCATTTCCAGGGTAGAAGCAACATTTTCACAGCAAAACTGGAAACTATTACTCTTATTCTGTTGTGTATCCA | 192 |
| | TCCTGTTGCCCAAAGTTCTCTCATTCCTGCATCAACCAAAGGAAAGCCAGTCCTACCTTGTCTCCAAGTCTT | |
| | AAAGATATCAGGATCAGGATTCCAAGGAAAAAACTTTAAGTGCCCCAGAAGTGCAAGTGCATTGCTTGCC | |
| | TTCTCTAATTCATCTTCAAACAGCAATGTATCACTAGGGCACCTCGATGCATCACCTGGAATTATCCTTTTA | |
| | GGTGGGAGAAGAGGTGACTCAGGGTCATAAGGCATGCTAAGACATCTTTCCCAAAAAGCAGCAAAAGTTG | |
| | TGAATGGTCGGCCATGTGCATCATTCACATCCCATGGTTCA | |
| GS437 | GCCAGTTTCTCCAGTTCCACGGCAAAATCCTTCATTACCTTCCTGTAATCTTCATCAAGATCAGGGATCTCT | 193 |
| | GAGATGTTAGAGACAGGAAGATGGCGCAAAAAGAAAGTGCTTTCCCAGTCCAAATCATTGATTTCAGATT | |
| | GAGCAGACTCCAAGCCTTTGCTTGCTACCATTTCTTGGAACCTTTGCTCCATGCTGCTCTGCAAGTTTCTCC | |
| | AGTTCCAGAGCAAATTTCTTCATTGTCTTCCTGTAATCTTGATCAAGATCTGCGTTGTCTGAAACGTTGGAG | |
| | AGTGGAAGATGGCGCAGGAAAAAGGTGCTTTCCCAGTCCAAGTCATTGATTTCTGACTGAACTGACTCAAG | |
| | ACCTTTGCTGGTCACCATT | |
| GS438 | TGACCCTGTCCCAATGTTCTCCTTCAAAATAAGTTCACTCCATGGAATATCCAGATCCTCCTTGTCAAGATC | 194 |
| | AACTTCATGACTTGGGTAATCCATTATTGACTGGTTTGCTTCATTGAAAGACTCATATTCCTCTGCACCAGG | |
| | AATCAAAGCAATCAGATGGCTGTTCCCACCCGCATAATTTGCACCCAGAGCTTCAGTAATTCATCCCCTTTG | |
| | CCACGTCATAAGCCATACCAAGCCTACGCCTCTCATCCAATACTTCTTTGGCGCCAGATCTATGCAAAAGC | |
| | CTATATAAGCTACCCCTAGATAAATATTCCGTGACAATTGATAAGTTAGGAGGCTGAGTGACTGCCCCCAT | |
| | AAACAAAACAATGTTTGGATGCCGTAAACGTT | |
| GS2108 | CATGTTGTTCAAAGTCAAAGTTGAAGGGAGTCATGCACACTGGTTCATCACTGATGTCATGCAGAGATGTC | 195 |
| | AAGTACGGGTGCGCCAGTGCATCTTCAACAGTAATCCTTTTTCTAGGATCAAAAGTTAACATTTTTTCAACA | |
| | AGATCTATAGCTTCAGGATGGACATGTGGAAACTTTTCCTGGAAAGATTGACGGCGGTAAAGGGGTAGTTG | |
| | CCTAATGTATCTCTTAGCATTTTCATTCAGGAACCCCAAATCAGCCTCTGATGGGGTGCCAATCAGCTCCAT | |
| | AAGTAGACGCAACTGATGCACATGATCTCTGCCAGGAAACAAAGGCTTGCGATCCATCAGTTCCATGAAA | |
| | ATACAACCAACAGACCATACATCAATTGCTGCAGTGTAATCAGAAGAGTTCAACAGAAGCTCTGG | |
| GS2109 | TGGGGCTCTAGCCTGGCGATGAAGCGGTGAACGTTCAATGCTGTACTCAGACCCAGTACTTTGCGTTGAAT | 196 |
| | GTCTTCTACGGTTATCAGCAGAACCTACTCCATGGCTTCTACTGCCACTCCTACTGCTTACATTGTCATGAC | |
| | GAGCTGGAGATTCGATAAAATTCTTGGGATCATCTTCCAAATGCAGTGATCCTTTTCCGGATTGATCCTCTG | |
| | AATTAGTTCTAGGTCTGGTAGGAGGTAG | |
| GS2110 | CTGGACTTTGTCATCGTTAGATTGGCCCCTAATAGCTTGTGGTCTCTCATTAGGCGTTCCCGGCACTTGTCC | 197 |
| | TGGGACACCCTGTCTCTCCTCCCTCACTTTGTTGAAAATGTGAGTAAAGCCATCTGCTGATGATGGGTTATT | |
| | CACATCCCAGTCACC | |
| GS274 | TACAAGCTTCTTGGCCTCGTCTATGACCATCCACAATCACAGCCAAAACAAAAGCAGCCATTGCGCGTTGC | 198 |
| | TCTGGATATGCTTCCATGCTGTCAAGAAACTTAATAAAATAGATATGACCACCATCCTTCACTAGATCAAC | |
| | CTGACATGACTTATCAAGTGCAAGAATCTTTGTCCATATGAATACAAGGATCTGTCGTAGTTCTGGTGTTGT | |
| | TGTTTGTAACAGCTTTAGAACATATGGGAATATTCCAACAGATAATGCCAGATCTACAGCCCATGGCCCCA | |
| | TATCAAGGAACCTT | |
| GS281 | TCATCCTGGCGTAAATCCTCATGTCCCTTCAATAAGAAGGCATAATCATCACCATCACTCCCATGAATGGTT | 199 |
| | AACTTCCTGGGTCGTTGCTTGGATGTTATGACAACAAGTTGGCGTGCAAATGATGCAATTGTAACCACTGG | |
| | TGCATCAGCACGGTATGACCCGGGAACAGCAAGCTCCAAATTGCGGCATTCTAGCAATTCTGGAGAAACA | |
| | GACTCCAAGTCTAAAGTTGTAAGACTTTGAAGTTGCTTATCTATCTTTCTGAAAACATGATAATAAATATCC | |
| | CAAGCCTGGGTC | |
| GS414 | CACAGACACCACAACAGCCTTACAGATACCCACTAGGCGACGACAAAGGGTGTCATATATAGCACTATTG | 200 |
| | GCTGAAGAATGCGCAAAACTTCCACCATGGAAAAATATGATGACAGGAACAACCTCAGAGCTCACAGGCT | |
| | TCTCAAGGTCAAGAATGTTCACCGAACGTTCTTCTCCTGGTCATTGTTTGGCAAGAAGTAGAAGCCAATAG | |
| | TGGCTTCCTTGAGAAAAAGAAGCTTGACCTGTTGTCCACAATCCTCAAGACCTTTTGCATATGCCAATTGCC | |
| | AATCCTGCAGAAGATCCAAACCAGCCACACAAACAAGACTTTTAGGGAATTTGAGTCCTTCAATGCTTCTC | |
| | CCCCTT | |
| GS415 | CAGTTAAGCCCGGACAGAAAACTGGAATGTTGTTCTTGTATGCCCAGTAAAGGTAAGAGCTTTCATTTTTT | 201 |
| | ATTTCTTTTCCCAATCGAGCTATTAACTTAGATGGTGTCCACAACACATTCTCATTATTTTGTTCCCCCAACA | |
| | TCTGATCAAAGATAGGAATAATCCAGTCCTCAAATTTGCAATAGTTGTCATTCGGGACCAATAAATTACCA | |
| | ATCCGATTCAACCCTTTTGAGCGCAGATATGCTCCAGGCAAAGAGAAATCTCCTTTATATGTTGGTGCCAG | |
| | GCACTTTATAAGATCTTCTTCAATGCCACCTGTAGTTGTAACAACTACATCAACCATGCGATGC | |
| GS416 | GCAACCCGCTTATGTTGTTTTGGTAAAGGATTGTGAGAAGGGTGGAGTCGGTGTGGGCGGCCAGACCCATG | 202 |
| | GCCCGATCCGGATCCGGACAAGTCGGGTAAGAGTTCAATTGCAAGGCTGCGCATGTCTTTTTGAATTGGCC | |
| | TTTGGACCCGGCCCATTTCAGGTCTTCCTTTGTAATACCCAGAGAATCCAACATCAGCCACATCAGCTTTCC | |
| | CACTAGCTTTTTCATGGCTTCATCATATCGCTTGACGATATCACAGTATTTGTGGTAATCTTGGGGCCAGAG | |
| | TTGACGAAAATGCTCAAGAGGGGATCCAACAATTGTGAATCCCTCAGACCACA | |
| GS425 | ACGTTCACCTTCATGCTCCTTTTCAAATTCCTTCTTTCTTTCCTTTTGTAGTTTTTTTCTTCTTTCACTTATCAT | 203 |
| | TTGCCTAAGTTCATGCACATCTTTCGTGATAAGATCCAAAGGTTGTAAGAATGAAAAATCTTCTTCCAGGTC | |
| | TGCATCACAATTACTCAAGTTGGCTTCCATCTCAAAGTACTGTAGACCCTTCTGAATAAATGTAACAAGAG | |
| | CACCTGACGGCACCAAATTTCCATCAATGGAGCATTTGTTAATACCAGCTTCATAACCAAAAGTAAAAGCA | |
| | GCATGCGTGAA | |
| GS439 | TTACCCTCAGCAAACCCATCCTTTATCTGAGTAAGCAAACTCTCATCAGTAGGAAGCTTCAGGTCATCCTTG | 204 |
| | GTGTTCCCATTTTCAGTAAGCAAGCTAAGAAATCCATCCTCAGCAATGTCAATCAACTGGTAATCAGTACG | |
| | ATTCACATGGGGAACATCACAGTTGTGAGAAGAGGGCACAATATCTTCAAGCTTCTTGGCAGTGAAAATAT | |
| | CAATCCCAACAAAGTGACACTTGGCATGACCGTGCTTACCAGTCTTGGAAGTAGAAACTTCAACAACCTTG | |
| | CAGGGACGACCCTTGATGACGATGTAACCGTTTTTGCGGATGGTTCCGGCTTGTTGAGGATAGGTCTTGGA | |
| | GGCTCC | |
| GS2107 | AAGTATGAATCCATGTGAAACTATGTGGGCTAGGAGCTTAGAGTAGTAGCTAGTGCGAATACAAAAGCCA | 205 |
| | TGGCAAAACAATATTACAGGGTATAAGCCAGGCACGGTTGGTGTAAAAATAAGCAATGGTTTTGGAGGTG | |
| | AGGAAGAAGCAGTGGATGTTTCAACATTGAATTGCTTCCAATGAATATCTCCCTTCTGAAAAACATCCGTG | |
| | GTGGCCAACGCTGGTTGAGCTCTCTGCGCCAT | |
| GS2111 | CACCTGAAGGGTCGGAACATACATATAACTTCGGGGGAGCTTTTAAACAGAAGGGCCACACAGACACAGA | 206 |
| | CAGCGGCTATTGCCACCATTGAGAGCATTGCAGGTCTGTATACCAAAGACCTGCCTACTGCAGTTCTGCAA | |
| | GAAATTCTCTTGTCACAAAGCTTACAGTGCCCTTGGCCACGTATTACTTCAGCCTTTCCAATCTCAAAGGTA | |
| | GTGGATAACTCATTTTGCTTCTCGTTTGTATTGCTCTCCGTCGTATTGCTTGGAATCTCAACCACCACATGA | |
| | GC | |
| GS5363 | AAACAATGGTGGCTTGTTCTTGGCTTGTGGCTGTGTTTTCTTTCATATCAAAATGAAAATCATAGGTTGAAC | 207 |
| | TCCCTTCCACGTGTTGATATGATCTTTTACTGTTTTCTGATGTTTCATTGATTACTCTGAGTGTCATCAAGGT | |
| | GCTCTGAAATGATCTTTCGAAGTTGCTTACAAAGTAATCAAATTTGTTGCCAAAAACATGAGACAATGATT | |
| | CTTTAACTGCCAAAGTGCATATCTGGGAGAGATGATCTATAGACATCTTTTCTCCAGTTGCTTCTTTCTTTTC | |
| | CAACACCTGAGCAACAAA | |
| GS5461 | GTGGTGCCCATCCTGGTCAAACAATGGTGGCTTGTTCTTGGCTTGTGGCTGTGTTTTCTTTCATATCAAAAT | 208 |
| | GAAAATCATAGGTTGAACTCCCTTCCACGTGTTGATATGATCTTTTACTGTTTTCTGATGTTTCATTGATTAC | |
| | TCTGAGTGTCATCAAGGTGCTCTGAAATGATCTTTCGAAGTTGCTTACAAAGTAATCAAATTTGTTGCCAAA | |
| | AACATGAGACAATGATTCTTTAACTGCCAAAGTGCATATCTGGGAGAGATGATCTATAGACATCTTTTCTC | |
| | CAGTTGCTTCTTTCTTTTCCAACACCTGAGCAACAAAGCCACAACATCGAGGACGG | |
| GS6396 | TCAGATTTGCATCAATTAGCCATGGCCTTTGTTCCCCTGGAGGCACCTTATTATTGTGATGATAGTACGAAA | 209 |
| | TGATATATGGAGCCAAAGTATGGTGATGATGAACATAGAGCAAATCGAACAGTACACTGTTCCTGAGTTGC | |
| | TCATCTTCCGATAATGTGTCTTCAAGCTTTTTCGTTTCAGCAAGTAACTTCTTCTCGTCGATGAACGGCAATT | |
| | TTACAACAGCCTGCCATGCAAAACGCTTTCCATTCATGTCAAGTTCAAAATCTGTTGGGTAAAAATCAGAA | |
| | ATTGGCGATGATGAATCCATCATTAGCATCCTGTACTTTTCAGGAAGAGCATTTGCACTTG | |
| GS6397 | ACATAATCCTTCCACATACTTCTGGACTACATCTTGTCTGACTTCATCAATACCCTCTGGAGTTGATACACC | 210 |
| | AAATTTCTCGGAATAGTACCTTTCCTTGTATCCAGGCTCCCCTAACTTCACCTTGTCCTCTGCCGGAGGCTG | |
| | TGGCTGAGCATTAACCTCATTTCTTGCTCTTTCATTATTTTCTATTGACTGCTTTATCCTAGCTCTCTTTTGAA | |
| | AGATTTGGTCTTCATAAACTGCTGCCGCCTGAATAAAATGTTCTACTCTATCCAAAAAAACCTCACCCGCAT | |
| | CTGTAAGATAACCACCCATTGTTGTAAACTCTCTTCTATAGATGCACATCAGCAGATT | |
| GS6403 | CAAAACACTTCTCCTGCTGACCTGGTGATGTAATCACCTCCCTCAATATTGAAAAATGGACTTCATGAGTA | 211 |
| | GCAAGAGACAGCATAATCAGATCTGCATCTAGGCCATAAAGACAATGACGGGTATTTGGGTCGAAGCCAG | |
| | GAAGGTTACAACTCTTCCCTCAACCTTTTTTCTTCAGCTTCAGCTTCAGCGGCATCTTTAGCGGCTCTAAAA | |
| | CGCCTGGTTCGCTGCTGGTTCATCTTAGCTCTAGGAGCCACTCCATCAATAGCCATATAGAGGAGTTTTCTT | |
| | GGACGGACTAATGAG | |
| GS6414 | ATAGAGCAAATCGAACATTACACTGTTCCTTAGTTGCTCCGTGTCCGTTAACGTGTCTTCAAGCTTCTTTGT | 212 |
| | TTCAGCAAGTAACTTTTTCTCATCGATGAACGGCAATTTTACAACAGCCTGCCATGCAAAACGCTTCCCATT | |
| | CATGTCAAGTTCGAAATCTGTTGGGTAAAAATCAGAAATTGGTGATGAAGGATCCATCATCAGCATCCTGT | |
| | ATTTTTCAGGAAGAGCTTTTGCACTTGCAGCTGGTAGAACACCCATTAGCTGATCGAAGGGCTTAAATGGT | |
| | TCCCCTGGGAAAAAAGTAATTTCCAAATCTGCTAGACCTTTGAGATCAGAAGCAAATGGAG | |
| GS5365 | TCTATACAGGTTTTAAGTAGCTCAGCATGTTTTGAGTCTTCTACTTGATTTTTGAACTTTTCAGTTTTGAAGG | 213 |
| | ACGTTTTAGAGAAACCAAATGATAGTTTGACCCTCTCCAAAAGGTTAGCATCAGAACTGAGAGCCAGCTGC | |
| | CTTTCTTTAAGTTGCAACTTCCTCATTGTAAGACCAATCTCAAATGTCTTAAGGTCATTCGACCTAGCTTGTT | |
| | CTGTGAGAGATCTTTCCAAAGTCCTATGCACAGATTGGCTAATCCCTGTATTGGATAAAATCATGCTTGAA | |
| | GAAGCAGAAGCCAACTGTTG | |
| GS5463 | GTGGTGCCCATCCTGGTCTCTATACAGGTTTTAAGTAGCTCAGCATGTTTTGAGTCTTCTACTTGATTTTTGA | 214 |
| | ACTTTTCAGTTTTGAAGGACGTTTTAGAGAAACCAAATGATAGTTTGACCCTCTCCAAAAGGTTAGCATCA | |
| | GAACTGAGAGCCAGCTGCCTTTCTTTAAGTTGCAACTTCCTCATTGTAAGACCAATCTCAAATGTCTTAAGG | |
| | TCATTCGACCTAGCTTGTTCTGTGAGAGATCTTTCCAAAGTCCTATGCACAGATTGGCTAATCCCTGTATTG | |
| | GATAAAATCATGCTTGAAGAAGCAGAAGCCAACTGTTGGCCACAACATCGAGGACGG | |
| GS6263 | TAATCTGACCATCTATTGCTGTCTCAATCTTCTTTTTCATTGGACAATTGGGAAACGTGCATTTGTAATAAC | 215 |
| | TTCTTGGATTTTCACTCCCTTTTACCTGTTTTTGTCCATATTTTCTCCAATTATACCCATCTTCTGATCTTCTC | |
| | TGTTCTCTTATGTATTGAGCTGGTTGGTAGGGAGGGTTTTGGTCTGTCTCATACTTTACAACGCTTCCTTGGT | |
| | TGTAATTCTCACCAGGAAACTCTGATTTCACCATAGCTGTAATTGAGTTCGAAGATCCATCATTCTTTTGAT | |
| | CGAAATTCCATGATTGTTGTTGGGGTTGTGCTGGTTG | |
| GS6264 | ATATTTCCTCCAACAATACCCATCTTCAGCTCTTTTCTGTGCTCGATTCATCTGAACTGGTTGAGCATTGTAC | 216 |
| | CCTCTTTGCATATTAGTACCAACATCTGTTTTTGTTGTGTTCATTTCATATGGCATGGTTTGATTAACAGTAG | |
| | TAAACTCGGATTTTGAATTGATTTGATCTTTGTTGTTAATCATCTCATTTTGCTTCTTCGGTTCATCCAAATT | |
| | CCACCCTTCAATTGAGTTGTTTGAAGAAGCATGGAAAGTAAAATTGTTGTTTCTTCCTTCATCCTTATTAGC | |
| | ACCTAGTTGAGTCCAATTGAATCCCTCACCACCAAAG | |
| GS6265 | AGATTTTACCGGAAAAGTCTTCGGAAAACTCTTGGTGGTGGTGGTGACAACTCTTATTATGATGAGAAGAC | 217 |
| | GAAGACGTTGAAAGGGGAGGTTTCCCAGAAGCAGCAAACCCAGACGACGATGTAGGAGGTGGAACAAAA | |
| | ACAGATCCACCGAATAGCTTTCTGTTAGACACGCTTCCTTCTCCGGTAATAGTAGACAAAAACGACGATGA | |
| | GTTCGCCGATGAAGATATACTAAAGGACGCCGAATTCGAAATCGACGCACATGGTTTTGTATTCGGGTTCG | |
| | GTTTTGTGAAATCCAAAGTCATCGGGTGGTTGTGCGACTGTC | |
| GS6266 | CCTCTAATATTGTCACATCTGAAGTAGAAATTTCACACTCCATGGAAGGTGATGATGGAGAGATATCCATG | 218 |
| | GGCTCAGGATCAGGACAAACTCTATTCTCTTCATATTTGATGGCAGGCTTCTCCTCTCGCTCAACAACTGTT | |
| | TCCATATCAGAAGGCACATCTTGTAACTTGGGTTTTTCCTCCTCCTCTTTGATAGATTTCTCTTCCCTATCTT | |
| | TCCCAACATTTTCTTCCTTCTCTTTCACTGCTTTTTCTTCCCTCTCTTTCCCAGATTTTTCATCCTTTACTTTTC | |
| | CAGCTTTTTCCTCTTTCTCTTT | |
| GS6267 | CAAGAGTGAGTTCAAGATCATCCATGCCTACATCATGAATCCTTTCCCCCTCCCAAGGGGTAACCTGCATA | 219 |
| | CTCTCAGCTTCAGCAACTCTTGCTCTATCCTGAATTGCATCATTCAAGGACATTTTCTGAACCATTGGCCTA | |
| | ACCAAGTTAAATGTGGGGGAATTTGGAATTGGAGGTCTGGAAAACCTTTGGAAGCTAATCCACTGTCCGGA | |
| | ATCAATAGTAGAGCAGTCAGATTCATCACATTCAGGTATGGTAGGCGGGGTAAAGTTCCTCC | |
| GS6268 | ATTTTGACGTAAATTTTGGGAAGCTGATGTTCTAGATGAAGTTACTGGTGCCGCTCTGGAATCTTTGTTTCC | 220 |
| | ATTGTTATTTCTCGGGAGTTGACTTTCAACAATATCATAATCTTTATTCCTGGGGACATAATTGATAGCCTG | |
| | GTGGTTTTTCCACATCATGGGAGAAGGCGGTTTTCTGGAGACAGCATCATTCTTAAATCTCTGAGACTCATC | |
| | AATCTCGTATTTGGGTTTATTTTGCTGTGAAGCTTGGCCAGAAACTCGTAAATGGAATGGATTAAGTTCCGC | |
| | AAAAAGATTCTTTGATTCATCATTATTACCTTGATTATAG | |
| GS6269 | TTACCCATTGGCGGAACCTTGACCTTTGCTTTGATGTATCGCTTCTAGACACTCCTTTGCTCTATGGACCTTC | 221 |
| | GATTGGATGTAGAAGCTCCCTTGCTTGGCATTCCTCTCAAACAAAACATCATATTTTTTCAAAATATCTTCC | |
| | CAGGCCGCATTCTCAGGAACGCCAAGAATCTGCCTTGCTTCTTGTTCTGTGATGGCTTTACTTGCTCTGCGT | |
| | ACGGTATTGTTCATTGCTTCCTGAGCTGC | |
| GS6270 | TAACCTTTATTTCGATACACTTCTTCCAGATGTTCCTTTGCTCTATGTACCTTCGATTGGAGGTAAAAGCTCC | 222 |
| | CTTGTTTAGCATTCCTCTCAAATAAAACATCATATTTTCTTAAAATTTCTTCCCAAGCGGCTTTCTCGGGGA | |
| | CTCCAAGTATCTGTCTTGCTTCTTGTTCTGTAAGAGCCTGACTTGCTCTACGAATGGTGTTTTGCATTGCTTC | |
| | TTGCGCAGCACCAGTTTTTGAGGCATTGACAATAGCTTGACGATAAGCTTGACCAACCGCCCGAATTAAC | |
| GS6271 | TGGTTGCGCCTTGGAAGACTTTAGCTTTCTTGATTTCTTGTCGGAAGTTTTCTTAACCTCACGTGATTCAACA | 223 |
| | TTTTTTGCGTCTTTTGCTGCAGATACATCTTCATGACACTCCTTTTCAGAGTTTTCCTCTAGAGTACCAGTTG | |
| | GTGTAAGCTTATTATCATTTGTTCTCTCAAACATCAAAATCTGATTTTCCCTAGGGCTCACAGATGATTCAG | |
| | AAAACTTGGGTGGTACAGAGTCAGCACTTAGCACACTCATCCCCAAATCCACAATCAAAGTAGGTTGTGCT | |
| | GTTTTTGAGTTTCTCTCTACCACCTGGCCTGATA | |
| GS6272 | CCTGACCCTCTTGTTGTGCTGGTGGTTGGTGGTTGTGCTCTCCTTCATACGTTGCCACTAACATTGACTGGTC | 224 |
| | TTCTAGACTTCTTTGTACCTTTTTCTTAACGGGGCAACCAGGAGCAAAGGAGCATTTGAAGTAAGCTCTAG | |
| | GGCAAGGGTTATCTCTTGTAACTTTTTGGCCATATTTTCTCCATTGGTATCCATCCTTAAGAATAAGGGTGG | |
| | AATCAGAAGCTTGAGTACGAACAGCAACCCTTGTGATCTTAGCTTTAATAGTCTCTTCTTTAGGTTTTCCAT | |
| | AGGAATCCTCTTCATCACTGGAACTACTCTCACCATTAT | |
| GS6273 | AATCAACATTTTATTAGTGATCTTAGGCTTGCTGCTAAGAAGAGTTACTCTTTTGAATGAATCATCGTAGCT | 225 |
| | AATACAACTCTGTTCTGTGTTCCCCTGTATTCCACACTCCACAGCTTCAATAAACTTCCTCTTCCTTGGATTC | |
| | TGATCTTCATTTTCGCCTTTCGACGTCGGACTAATCAAACCAATCAACTGATTTTGCAGCATACTGTAATTC | |
| | TTACACATGGATGCCAACATTTTTGTCAGCTTCTTATTCTCTATGTTCATCCTGTTCAACTCCTCTTCTAAAT | |
| | TCCCACTTTTTCTCTCGTCCACCTGTGTTTTGTCAT | |
| GS6274 | CTTCAAAGCTACAAGCTAGGTCCATGACTTCTTTTGCCTTGTCAGTCGGCAAGTCATTAAAGACCAAGACTT | 226 |
| | GCCCTCCATAGAAGATGGTCATTTGGGGAGTCGGAGGCTCGGGGTTAGCACGCTTGTTCACGCTTGAATTA | |
| | AACCCAGAAAGCTGAGGAAAGAGATTGATTGTTTTCCCAGCACAAACATTTGAATCTTCAGCTGCATGTTT | |
| | CCCACTCACAGGGAACAAATTCATCGTTGAAGTTGGTCCTCGGGACTCGATGTTTCCAGTAACACCTTGAG | |
| | CGCGTTTCTCCTTAACATACTGGCTGAGAAGGTTACAAGTC | |
| GS6275 | AGGTAGCTTGGATTCGATGCTTTCGCTTTTCGAGAAATTGGCGTAACGATTTCTTCATGGCCGATAAACCAT | 227 |
| | TTTGCGTTTGAGTACATAATTGTTGAGATTGTAACGTTGGTGTTATTGTTGTAGATGTACATATTTCTTGTAT | |
| | CGTTTTTCTACTTGCTAACTGTATTATGCTTTTTGCCTGAACATCGGTAACATCGCAAACGCAGATAGTTCC | |
| | ATTATAAAATATCGTCCTTTGTTGTTCTTGCTTTTTTGGATTATCTCCATTAACAGATCCCATGGAGATCGTA | |
| | GAAGCTGTTGAAGAGATCGACGTGGAAGATGGTGATA | |
| GS6276 | TGTTCTATGTTTGTATATGGCGACACCAACCTCTCTTTGCGCTTCTCAAAGAACCTCTGCAATGACTTCTTA | 228 |
| | CGTTTAATAGGCATATCAGAATCATCTCCATCATCATCATTAAGTCGTCCGAGGATATTCTTAGTAGTTGCT | |
| | TGGGATGACTTAAAACATGAATAATCCACCACCTTCCCTTCGTTTTTTGATTCATTCTTTTCCATTGCAAGCT | |
| | TCATTATGCTTTCGGCCTTGTCGTTGGGTAGATCATAAACAGAAACTGACCCATTGTAGAATATGGTCATTG | |
| | GGGCAGTTTCTTCTGCTTTCTCCATGCTATCTTCCCCAG | |
| GS6277 | TGAAGAATTCAACAATAACTCTGGTGCCCGATACCATCTAGTCACAACATATTCAGTCATAAAATCAGTCT | 229 |
| | CCGATGTTACTCGAGCTAGTCCAAAATCACAGATTTTTAAATCACAGTTGGCATTAAGCAAGAGATTGCTA | |
| | GGCTTTAGATCCCTGTGTAGAACATTTGCAGAGTGAATGTACTTCAATCCACGTAGTATCTGGTAGAGAAA | |
| | ATACTGACAGTGCTCTTCTGATAATCCTTGGTTTGAACGGATAATTTGATGAAGATCAGTGTCCATCAGCTC | |
| | ATATGCAATATATACATCATTAAATGCCTCCCTTCGAGGAG | |
| GS6278 | CTTCCAATTTATCAGCTTCACCTTCCGCTATAGGATAAACCTGTTCACCCAACTTCAAATTGAGTGGTCTAG | 230 |
| | CTTCATCATTCAACTCCTCCTCGGGAACAACCACACCCCTCCTTCTCTTCTCCATTTCCCTTTTCCCTTTCTC | |
| | ATTAGTTTTATTAATTTCTTTAGAACAGACATTGGTGTTAGCCACAAGTTCTGGTCCAAGAGGAAAAAGCT | |
| | GCTTACTTCTACAATCTAAAGGTGTAGAATTCAATGGACTGGCAGTAAAAAGATCACCATCCCATTTCCAA | |
| | TCATTCAAATCCCATTCTAAAGTCCTTTTTCCTACCGTCTT | |
| GS6279 | TTGAATTCATTATCCAAATCATCAACATCCTCCTCTTCATCATCGCCCTGCACTCGAAGACTCCCTTTCTGTC | 231 |
| | TCTTGTATCTTGTCTTGCATTGGGGGCAACACTGGTTTCCATCTTTTCTCTCATACTCATAACAAGGCCTGCA | |
| | AACTGGAAAAGCACATTCATTGCAAGCGACAAAGACACCACCATTCGAAGTAACTCCCACGGTATCCCCA | |
| | CAGATCTGACAGATTTGTCCATCCAAATTCTTCAAAGATTTGGACCCACTATCAGTACTATCATGGCGAATT | |
| | CTAACAAGTTCGTTCCTTTTGTAAGATCCAGCCACCATTC | |
| GS6280 | ACAAATACTTCTGATTTACCAAACCGTTTCTCTAAGCTTATTTGTGACATTAGTAGTGACTTTTCATCGTCA | 232 |
| | AATCCAGCACCTTCAACTCCTTCCTCGATATCTTCCAGATTAAATATTGGCACAGTAGGGTCAGCATGTTTT | |
| | GATTTCTTCTTGGAGTCTTTCTTGCTTGATTTCTTCCGAGATCCACCAAAGCATGATGATAGGATACCTTTCT | |
| | TCTTGGGCTTTGGTTTGATAGGAGGCTCATAACCATACAAAGCAGTTCTGTTGAAGACACATCCCGTACCC | |
| | ACATAGACAGGTCCTTGGATACCATCTAAACCTCTCAAAT | |
| GS6281 | TTGAATTCATTATCCAAATCATCAACATCTTCCTCTTCATCATCTCCCTCCACGCGAAGACTCCCTTTCTGTC | 233 |
| | TCTTGTATCTTGTCTTGCACTGGGGGCAACACTGGTTTCCATCTTTTCTCTCATACTCATAACAAGGCCTGC | |
| | AAACTGGAAAAGCACATTCATTGCAAGCGACAAAGACACCGCCATTCGAAGTAACTCCCACTGTATCCCC | |
| | ACAGATCTGACAGATTTGTCCATCCAAATTCTTCAAAGATTTGGACCCACTATCAGTACTATCATGGCGAA | |
| | TCCTAACAAGTTCATTCCTTTTGTAAGATCCAGCCACCATTC | |
| GS6282 | ATTATAAGCAAATCAGCAACAAGAAAGTCTGCACATGTTCTGAGGAGCTCACTATGTCTTGTCTCTTCCAA | 234 |
| | TTGATTCTTAAACTTCTCGACTTTGAAGGATGTTCTTGAAATTCCTAGAGACAATTTACATCTCTCCAACAA | |
| | ATTCAAATCAGAATTAAGAGCCAGTTGAGTTTCCTTCAGCCGCAACTTTTTCATAATTAGACCTATTTCAAC | |
| | GCTCTTTAGGTCATTTGCACGCGTCTGCTCAACAACAGATTTCTCAATGGTACTGAGCACTGAATAGTTGCT | |
| | CAAGTGATTTCCCATATTTGAAACAACAATTTGTGAGCTTA | |
| GS6283 | AATACAATATTACCATATTGGTCTAAATCTTCGTTATAAGAACCACCGGAGAACTCTTGCAATCTCGTCGCT | 235 |
| | GTACGAAGAAATTTTCCAAGATGAACAAGATCAGTATCTCCAATTTTCACAGAGACTAAAGGACAATTTCT | |
| | GGCAAGAAGTTCAAGATCTTCAACTGTGATTTTCTCCATAAAAGTCATATAGAAGTTCAGAGTTTCAAGAA | |
| | CTATATTGTGTAGTGCCAGCTGGTGAAACCATTCACCATTAGTCTCCTCGACACCACTGTCTTCCAGAAACA | |
| | GTGTTCTCAGGTTTCTGCAAGAGCTAGTGATACGCAAGAG | |
| GS6284 | TTCACCATCTTTAGTTTGAACTTCCAAACCATTAACTTGATTCTGATGAAGTATTGTTATGAAGTTTTTGTCA | 236 |
| | GTGTGACCAACTGCACCAAGATTAAGTTCATCCTTGAGTGGTACCCTGTACTTTGCTATACGAAGAAGGTA | |
| | AGTGGTCGAGTTGAGATGAGAGTTAAGGTATTTCTCCACCCCGTAAGTCTCAAATACCATCCTCATTACCA | |
| | ATTGTTCTAGCTCCGATACTGTCTTTGCATATTCATGGATGGTTTCACAAAAATGATCGTTCCTAGTAGGCC | |
| | ACAAGAGATTAGTGAAAGATTGGATGTTATGAAGCGTTGTT | |
| GS6285 | CATTCATGGCTCTTATATCTTGAACTACGTCAACAATTAAACCGGGATTCCGTATTGAATGAAAATACAAC | 237 |
| | ATGTCTCCAAGTGATCCATCTGTTAAACCTGGGCAGAATACTGGAATATTATTCTTGTAGGCCCAAAAAAG | |
| | GTATGAACTCTTGTCATTTATTTCTTTTCCCAAACGAGCGATCAACTTTGAGGGTGTCCATGTTATCTTCTCT | |
| | GTATTTTGCTCTTCCAGCATTTTATCAAAAATTGGAACAATCCAATCCTCAAATTTGCAGTAGTTGTCATTT | |
| | GGGACCAATAAGTTACCAATGCGATTCAATCCTTTCGAGC | |
| GS6286 | TATCTCATTTTCCAGTCGTCAATCATTTCATTAACAACATCAATAGCTTCTCCGAGATTCGATGCTTGGAAA | 238 |
| | CCGGTGGAGACAAACGATTTAAAAAGCTGGGTATAACTCTCAGTCTTAATGCATTTCCCTTCTAGGGTTTCT | |
| | GACTCCTTGAACACCACAGAGCGAACCGACGCCAAAATATCACTATCACCCATATTTCTACACCTGAAATT | |
| | CCTTTGTAGTAGAACGAATGAGATAGGTTGGATAGGATCATATGATCGGAATTGTAACCTCATCTG | |
| GS6287 | ATGTCCTTGAGGGCATTAATTTGTTCTTCTCCCATGGCACTCATCACGCTCACAATCAGGTCTTTCCCCTCA | 239 |
| | GCAAATCCATCCTTGATCTGTTTATTCAGAACTTCATCAGTTGGCAGCTTCAAGTCATCCTTAGTATCTCCA | |
| | TTATCAGTCAGCAGGCTAACAAAACCATCCTCAGTAATGTCAAGGAGCTGGTAATCAGTACGGGTGACATG | |
| | GGGAACATCACAATTGTGAGAAGATGGAACAATATCCTCAAGCTTCTTCCCAGTGAAGATGTCGATACCGA | |
| | CAAAGTTACATTTAGCATGTCCGTGCTTGCCAGTCTTAGA | |
| GS6288 | TTAGATCCACCACCAATATCCTTGACGCCACAAATTTGTTCTTCTCCCATGGCATTCTGAACACTCACAATC | 240 |
| | AGGTCTTTTCCATCCGCAAATCCTTCCTTGAGCTGCTTAAGAAGAGTTTCATCAGTTGGCAGCTTCAAGTCA | |
| | TCCTTTGTACCTCCACTCTCAGTCAGCAGGCTAACAAAACCATCCTCAGCTATGTCAATGAGCTGATAATCG | |
| | GTACGATTGACGTGGGGAACATCACAATTGTGAGAAGATGGAACAATATCTTCAAGCTTCTTCCCATTGAA | |
| | GATGTCAATACCAACAAAGTTACACTTAGCATGACCGTGCT | |
| GS6289 | TTAAGCTGTTTCAGCAGAGCATCATCAGTTGGCAGCCTCAAGTCATCCTTAGTGTCTCCATTGTCAGTCAGC | 241 |
| | AGGCTAACAAAGCCATCCTCAGTTATGTCAAGGAGCTGGTAATCAGTACGAGTGACATGGGGAACATCAG | |
| | AATTGTGAGAAGATGGAACAATATCCTCGAGCTTCTTCCCATTGAAGATATCAATACCAACAAAGTTACAT | |
| | TTGGCATGGCCGTGCTTGCCGGTCTTAGAAGTGGAAACTTCAACAACCTTGCAGGGGCGATTCTTGATGAC | |
| | GATGTAACCGTTCTTACGGATAGTACCAGCTTGCTGAGGGAAAG | |
| GS6290 | GAGCATCTCTTAAAGATTCTGCTTTTGCAACTAAAGCAGTAACTGAGACACTTCCTTCTTTCGTAAACTTCA | 242 |
| | CAGCATTACCAATAACATTCAATAGAACCTGCATTAGACGTTTTTCATCACCAACAGCAAACTCTGGCACA | |
| | TCCGTTGCCAAATTCGTCGTTATCAACAACTTTTTGATAGATGCTATGGGCTTGATCAAATTAAGGACCTCT | |
| | CTAAACATCGTGTGGAGATTAAATACTCCATTATCAAGCTTCAAACTTCCATCTTCAAGCCGTGAAAGGTC | |
| | CAAAACATCATTTATCAGAGTCGCAAGAAGATTACTGCTTT | |
| GS6291 | TTTACATTATCATCATCAGCAATCAACACTTTCAACCCTCTGAACCGAGAGTTTGAAGAAACTGTGTGCTCT | 243 |
| | GTCATATATAAGTTTCCTTCAGGAATATATCTTTGTTGTTGAAATCGTAGAACAAGAGTCATGTTCTGTGGT | |
| | ATCCCATTTGAGTTTAGAGATATCCATATATCACCCTGCATCATCTGAGCAAGCCTTTTGCACATGCTGAAG | |
| | CTCAATACTTCCTTGTTTGAATCATTCTGGGTTCTCCTAATAGTGTCTGTCAGATAACCTCCACTTATGTCAA | |
| | ACTCAAACTTTACTTTAATGTACTCCTCGACAGTAGCCG | |
| GS6292 | GTTGCTGAAACTCTGAAAATTCCTTCCTTTTAAACCATGAGTCAAAATGATCTCAGTCCTATTCTCATTGGG | 244 |
| | CATCCAAACAGCACAAGTAAGCAAATCCAAGGTTTTAGAGAGCTCAACCATAGTTGTGTGCAAGATTGTAT | |
| | GCCTATCTAAGGACTTGCGAATCTCATGAGTAAGCATCCTAACATGCCATCCAGCTTCACTTTGTTTCTTAA | |
| | TCATCCCAACCTCCCTACCCAAATCCCAAGTCTTCTTCTTTAACATGAATTCTCTGACCTTGACTTTGAGAA | |
| | GCAAAGGGATGAGGGTGATAAGGGTAATGGCAGTAGCACAA | |
| GS6293 | TTCACTTCTACTTGAAACTAGTTGACTAGCCTCAAACCGAAAATCTTTTGATGCATTTCTCGCTGAATCAGT | 245 |
| | TAATGCCTGGGTCCCTCGTCTGCTAGGAGGAACAAATCTTGATGGAAGAAACTCTCTGATGGCATTAGATG | |
| | CTCTTCCAATATTTTGTGGAGGATTACTAGATTCAGAACACTGGGGTTCTCTATCATGACCAACAAACTGA | |
| | GAATGTACTTGGTCATTTGGTATAGGATTGTTTACGTCGGGCAGCTTTCTACCAAGTTGCTTAAGATACATA | |
| | GGATCTCCTGCATCTTGCTCATTCACAACTGCACCTGTAGAA | |
| GS6294 | AGAGTGTACATATTTTAAGCCACGTAAGAGCTGATATAAGAAGTACTGGCAATGATCATCAGTCAAAGCTT | 246 |
| | GGTTAGAGCGAATTATCTGATGAAGATCAGTATCCATCAGTTCATAAACAATGTAAACATCATTGAATGCT | |
| | TCTCTCTTTGGTGGTCGAATGATATCCTTAATCGCAATAATATTTTCATGATCCATATGGCGAAGTAGTTTG | |
| | ATCTCTCTCAGTGTCCTTTTTGCATCTACTATATTGTCGAATGCATTACCAATCTTCTTGATAGCAATTTCAT | |
| | GGTGTGTTTCTGAATTAACAGCAGCACAAACAAGGCCAG | |
| GS6295 | CGAGTGTATATATTTTAGCCCGCGTAGGATTTGATATAAGAAGTACTGGCAATGATCTTCTGTGAGTTCCTG | 247 |
| | GTTAGAGCGAGTAATTTGATGAAGATCAGTATCCATTAATTCATAAACAATGTAGACATCATTGAACTTCT | |
| | CTCTTTCAGGTGGTCGTATGATGTCCTTTATCTTGATAACATTGTCATGGTCCATGTGACACAGAAGTTTGA | |
| | TTTCTCGCAAAGTCCTTTTAGCATCAATTCTATTGTTAAAAGCATCTGCAATCTTCTTAATGGCGACCTCTTC | |
| | TTGTGTCTCTGAATTTCGTGCACAGCAAACAATGCCGTA | |
| GS6296 | AGAGTGAATGTACTTCAACCCTCGTAATATTTGATACAGAAAATACCGGCAATGATCATCTGTCAACTGCT | 248 |
| | GGTTGGATCTTATTATTTGGTGAAGATCAGTATCCATGAGTTCATGAACCATGTATACATCATTGAAGTTCT | |
| | CTTTCTGAGGAGGTCGTATTAGGTCCCTGATGGAAATAACATTGTCATGATTCATGTGGCGAAGAAGTTTG | |
| | ATTTCGCGGAGTATTCTTTTGGCATCAATTCTGTTATGAAATGCATTGCTAACCTTCTTGATTGCAACTTCTT | |
| | CTCGTGTCTCTGAATTGACAGCAGCACAGACAATACCATA | |
| GS6297 | ATCTTATACCTTGTATCCCAATGCAAATGACCGCCTTTCTTACCGTGTATCACCTCCCCTAAGCTTCCATTA | 249 |
| | GGCATGTACTCATACACTAACAGATTAGTCTCATGGTTGGAGCAAAACCCTAACAATCTAACAATATGCCT | |
| | ATGTCTAATCTTCCCTAAAGTCTGAATCTCAGCATTAAACCCATGATCATGAGATGAACCCCTACTCATTAC | |
| | AGGAAGACGCTTAACAGCGACTTGATCACCATTTGGCATATTTCCTTTGTAAACAATCCCAGCACCACCTTT | |
| | CCCAATAATGTTATCTTCCTTCAAGCAATCCAACACATCAT | |
| GS6298 | ATACTGTAATTCTTACACATGGATGCCAACATTTTTGTCAGCTTCTTATTCTCTATGTTCATCCTGTTCAACT | 250 |
| | CCTCTTCTAAATTCCCACTTTTTCTCTCGTCCACCTGTGTTTTGTATAAGGGTGGAATCAGAAGCTTGAGTA | |
| | CGAACAGCAACCCTTGTGATCTTAGCTTTAATAGTCTCTTCTTTAGGTTTTCCATAGGAATCCTCTTCATCAC | |
| | TGGAACTACTCTCACCATGTAACCTGCATACTCTCAGCTTCAGCAACTCTTGCTCTATCCTGAATTGCATCA | |
| | TTCAAGGACATTTTCTGAACCATTGGCCTAACCAAGTTAAATGTGGGGGAATTTGGAATTGGAG | |
| GS6299 | ACTTAAAACATGAATAATCCACCACCTTCCCTTCGTTTTTTGATTCATTCTTTTCCATTGCAAGCTTCATTAT | 251 |
| | GCTTTCGGCCTTGTCGTTGGGTAGATCATAAACAGTAATTGTTGAGATTGTAACGTTGGTGTTATTGTTGTA | |
| | GATGTACATATTTCTTGTATCGTTTTTCTACTTGCTAACTGTATTATGCTTTTTGCCTGAACATCGGTAACAT | |
| | AGGCAGATCAGAAACATTAGGCACGGTAGCTGGTATGACGTTCTTCACAAATTTTGGAGCAACAGTCAAGT | |
| | TCTGGTTTGATTTCTGGGTTGGAATCAGGGTCTGA | |
| GS6300 | TATCTCATTTTCCAGTCGTCAATCATTTCATTAACAACATCAATAGCTTCTCCGAGATTCGATGCTTGGAAA | 252 |
| | CCGGTGGAGACAAACGATTTAAAAAGCTGGGTATAACTCTCAGTCTTAATGCATTTCCCTTCTAGGGTTTCT | |
| | GACTCCTTGAACAGAATACTGGAATATTATTCTTGTAGGCCCAAAAAAGGTATGAACTCTTGTCATTTATTT | |
| | CTTTTCCCAAACGAGCGATCAACTTTGAGGGTGTCCATGTTATCTTCTCTGTATTTTGCTCTTCCAGCATTTT | |
| | ATCAAAAATTGGAACAATCCAA | |
| GS6301 | TAATCAGTACGAGTGACATGGGGAACATCAGAATTGTGAGAAGATGGAACAATATCCTCGAGCTTCTTCCC | 253 |
| | ATTGAAGATATCAATACCAACAAAGTTACATTTGGCATGGCCGTGCTTATCCTTGACGCCACAAATTTGTTC | |
| | TTCTCCCATGGCATTCTGAACACTCACAATCAGGTCTTTTCCATCCGCAAATCCTTCCTTGAGCTGCTTAAG | |
| | AAGAGTTTCATCAGTTGGCAGTCTTTCCCCTCAGCAAATCCATCCTTGATCTGTTTATTCAGAACTTCATCA | |
| | GTTGGCAGCTTCAAGTCATCCTTAGTATCTCCATTATCAGTCAGCAGGCTAACAAAACCATCCTCAG | |
| GS6302 | TTTGATTTCGCGGAGTATTCTTTTGGCATCAATTCTGTTATGAAATGCATTGCTAACCTTCTTGATTGCAACT | 254 |
| | TCTTCTCGTGTCTCTGAATTGACAGCAGCACAGACATTAATTCATAAACAATGTAGACATCATTGAACTTCT | |
| | CTCTTTCAGGTGGTCGTATGATGTCCTTTATCTTGATAACATTGTCATGGTCCATGTGACACAGAAGTTTGC | |
| | TTAATCGCAATAATATTTTCATGATCCATATGGCGAAGTAGTTTGATCTCTCTCAGTGTCCTTTTTGCATCTA | |
| | CTATATTGTCGAATGCATTACCAATCTTCTTGAT | |
| GS6303 | ATCATTGATCAAAAGAGAAAGGACATTCCCTGTCTTCATCATTGTGTCAACAATAAGTTTCTGTTCACTGCT | 255 |
| | TAAATTCTCATCTTTTAGAACTGAAAGCAACCCCAAATACTTCCTTGTTTGAATCATTCTGGGTTCTCCTAA | |
| | TAGTGTCTGTCAGATAACCTCCACTTATGTCAAACTCAAACTTTACTTTAATGTACTCCTCGACAGTAGCCG | |
| | TAAAGCAGTAACTGAGACACTTCCTTCTTTCGTAAACTTCACAGCATTACCAATAACATTCAATAGAACCT | |
| | GCATTAGACGTTTTTCATCACCAACAGCAAACTCTGG | |
| GS6304 | GGCATGCAGTAGATCGACATCCACCCACGAGCATGCATCTTAAATCCAGTCAAAATATCTTCTGTCACTGA | 256 |
| | ACCATATATCCACCCAATCTCTTTACCCCATTCAGACTTGTCCTCGTATCCACAGCTAATAACATGAATGGC | |
| | TTCTTTCTCCGAGATCCACCAAAGCATGATGATAGGATACCTTTCTTCTTGGGCTTTGGTTTGATAGGAGGC | |
| | TCATAACCATACAAAGCAGTTCTGTTGAAGACACATCCCGTACCCACATAGACAGGTCCTTGGATACCATC | |
| | TAAACCTCTCAAAT | |
| GS5364 | AGTCTGCTGACTGAATATCTTCTACCTCTTCAACCATTTTTATACGACGCTGAGGCCCAGTAGCGCCATTTA | 257 |
| | TTGATCTTCTGCAATCCTCTATAACATCACTACTTCTTGCGGAACAATCTCCTTTGTTAAAGGAGATGGATT | |
| | GGGTTGCATCAGAAATACAATCGCCAACAATGTTATTCCTAACGTGATCATCTTTATTGTCTATAGATGAGT | |
| | TATTTATCAAAGTCAGAGTGCTGAGGGTACTTCCAAAAGATTTCTCGAAGTTTCTTGCAAAAGCATCAAAC | |
| | TTGTCGCCAAATACATTGGTT | |
| GS5462 | GTGGTGCCCATCCTGGTCAGTCTGCTGACTGAATATCTTCTACCTCTTCAACCATTTTTATACGACGCTGAG | 258 |
| | GCCCAGTAGCGCCATTTATTGATCTTCTGCAATCCTCTATAACATCACTACTTCTTGCGGAACAATCTCCTT | |
| | TGTTAAAGGAGATGGATTGGGTTGCATCAGAAATACAATCGCCAACAATGTTATTCCTAACGTGATCATCT | |
| | TTATTGTCTATAGATGAGTTATTTATCAAAGTCAGAGTGCTGAGGGTACTTCCAAAAGATTTCTCGAAGTTT | |
| | CTTGCAAAAGCATCAAACTTGTCGCCAAATACATTGGTTGCCACAACATCGAGGACGG | |
| GS6562 | AGCTGCATGAAACTCAAACTGTTGTTTCCATTCTCCAGTCTTAATGTCCCATACAATTGCTGTTTTGTCTAC | 283 |
| | ACTGCCACTTAAAAGATAGTCTCCTTTCTTGTTCCATTTTAAAGAGAATATTGGCCCCTTATGTTTTGTTAAT | |
| | GTACTCATCAGCTCCCCATCTTTACTCCATATTCTGGCTTGTCCATCATAAGAACCTGTTGCAAGTAAAGTT | |
| | CCATCACCATTCCAGTCAAGTGTGGTTACATCTTTGCTCTTATCATTTGTTCTACCTCTGTAGTGTTTCAAAA | |
| | CAGCAACATCTAAGGGGCCTTTTTGTAA | |
| GS6563 | TCCTTTGTAAAGAGAATTTCCGATTGCTGATGAAGGGATGTGATCTAATTGACTAGTGTTGTTTTGCACTTC | 284 |
| | AATGTTTTGACTACTGGCACCATTGATATTATTGTTTGAAACGGGTCTTGAAGGAAGTGAGAAATCTTTAGT | |
| | TGACTGGACACCTGGAAGTTGACTTGATTTTGGGATGTTTGGTTTCAAGGAGGAATCTTTTCCACTGAAAAT | |
| | ATCCGCGGGTATAAGTGTTCCAGGGGCTGCCATTAGATCCACTAGAAACTCACTTTCATTATCCAACTTGAT | |
| | AATGTTGACTGCATCATCCTCAACACCAGT | |
| GS6564 | GAGTACCTGCATATTCACCTGCTTGATAAGTTGTTTCTAAACATTCTTTAGCCCTATGAACTTTTGACTGTA | 285 |
| | GATAAAAACTCCCATTCTTGGCGTTCCTTTCAAATAAATTGTCATATTTTTGTGCGATTTCTTCCCATGATGA | |
| | TTGTTCATTGACACCAAGAATTTGTCTGGCTTCTTGTTCAGTCATAGCTTTGCTTCCTTTACGCACAACATTT | |
| | TGTAATGTGTCTTGAGCAACACCTGATTTTGATGCATTTTGGAGGGCTTGTCTATATGCTTGAACAAGAGCC | |
| | CTAGCCATTATTCCAGA | |
| GS6565 | TTTGACTGCAAATAAAAACTCCCATTCTTCGCATTTCTCTCAAATAGATTATCATATTTCTGTGCAATCTCCT | 286 |
| | CCCAAGATGATTGTTCAGTCACACCAAGAATTTGTCTCGCTTCTTGTTCAGTCATAGCTTTGCTTCCTCTTCG | |
| | CACAGCATTCTGTAATGTTTCTTGAGCAACCCCTGACTTTGAGGCATTTTGGAGAGCCTGACGATATGCTTG | |
| | AACCATTCCCCTAATCAAGATTCCAGAGCCCATTACAATCAAATTTGCAAGAATCTTAGCAGCCAT | |
| GS6566 | AACTTGTTGTGTGTTGTTATCTTGAGGGATTTGGTTAACATTACTAGACATAGATACAGGCTCTTCCATGGC | 287 |
| | TACTCTAGATTTGGTCTCTTCTACTTGAGATGTTTCCATTTCGACCTCGTTGACTTTGGTCAAATCAGTTTCA | |
| | GGTTCCTTTGGCTGCTGCTGTTGGGGTTTAGATACTGTTTTAGCCATGTCAGACGATTGTGACTTTGAAGAC | |
| | TTCTGTTTCTTTGACTTCTTTTCAGTGGCTTTTTTCACAGTTTCAACAGGCTTTACTTCTTTCACAACACTGG | |
| | ACTCATTAGTAGATGGTTTCTGGACCTT | |
| GS6567 | AATCACATATTTTTAAATCACAATTTGCATTCAATAAAAGATTGCTTGGCTTTAAATCCCTGTGTAACACAT | 288 |
| | TTGCAGAGTGTATGTACTTTAACCCACGAAGGATTTGATACAGAAAATACTGGCAATGTTCCTCTGATAAA | |
| | GATTGGTTAGAACGGATTATTTGGTGGAGATCAGTGTCCATGAGCTCATAAGCAATGTAGACATCATTGAA | |
| | GGATGTTCTTTCTGGAGGTGGGATTATATCTCTAATTGCAACAACGTTTTCATGATCCATATGACGGAGAA | |
| | GCTTGATTTCTCGCAGTGTCCTCTTCGCATC | |
| GS6568 | ATCGAACAGTTTGGATACGTGCACTTGTAATAGCTTCTAGGATTTTCACTTCCTTTCACCTGCTTTTGCCCGT | 289 |
| | ATTTTCTCCAATTATACCCATCATCTGATTTTTTCTGCGATGGTTGATTGTTCAAATTGTTGTTGCTGGCTGC | |
| | ATAAATTTCCTTTGTAAACGATTTCCGTGATATGTCCTTCTAAGTTTCTTTCAACTTTCTTCTTTGTTGGGCA | |
| | ATTAGGGAACGTACATTTGTAATAACTTCTAGGGTTTTCGCTTCCTTTGACTTGTTTTTGACCGTATTTTCTC | |
| | CAATTGTA | |
| GS6569 | TATATTTTAGAATTCCCACTTCCAAGAGTGAGTTCAAGATCATCCACTCCAACATCATGAATCCTCTCGCCT | 290 |
| | TCCCATGCCTTCACTCCTCCATTCTCAATATATTTGAGCATTCCCACTTCCAAGGGTGAGCTCAAGATCATC | |
| | AAAACCGACTTCATGAAACCTCTCGCCTTTCCATGCCTTCACCCCTTTACCCTTTTAAACGTTGATATCTAG | |
| | TTGGGCTTGTGGGAGCAGAAGATGCAAAGTAGGGGAGATTGAAAGAGGGAATTGACTGTTTGGTGAATGA | |
| | TTCCCAGATTAGAT | |

*Each thymidine (T) within a nucleic acid sequence (dsRNA sequence) provided in Table 2 can be replaced with a uracil.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A polynucleotide that inhibits a plant gene involved in suppression of a systemic or localized responses to a pathogen.

2. The polynucleotide of claim 1, wherein the plant gene is involved in suppression of a systemic response to a pathogen.

3. The polynucleotide of claim 1, wherein the plant gene is involved in suppression of a localized response to a pathogen.

4. The polynucleotide of any one of claims 1-3, wherein the plant gene is involved in inhibition of a systemic acquired resistance (SAR) pathway.

5. The polynucleotide of any one of claims 1-3, wherein the pathogen is a fungal pathogen.

6. The polynucleotide of any one of claims 1-5, wherein the plant gene is selected from the group consisting of MPK4 (Mitogen-activated protein kinase 4), CPR5 (Constitutive Expression of PR Genes 5), MPK4a (Mitogen-activated protein kinase 4a, RLK3 (Receptor-like kinase/leucine-rich repeat receptor-like kinase), NPR3 (NPR-1 like protein 3/salicylic acid receptor), RFC3 (Replication factor C subunit 3), SNI1 (Suppressor of NPR-1 1), CEV1 (constitutive expression of VSP 1/cellulose synthase family protein), COI1 (coronatine-insensitive 1), RAP (RNA-binding domain), DMR6 (Downy mildew Resistance 6), DMR6-like (Downy mildew Resistance 6-like), ARR9 (response regulator 9/two-component response regulator-like protein), NPR4 (NPR1-like protein 4/salicylic acid receptor), NAC25 (encoding NAC domain containing protein 25), BRI1a (brassinosteroid insensitive 1a/Leucine-rich receptor-like protein kinase), BRI1b (brassinosteroid insensitive 1b/Leucine-rich receptor-like protein kinase), TAR2 (tryptophan aminotransferase related 2), YUC2a (Yucca/indole-3-pyruvate monooxygenase), ETR1 (ethylene response 1/ethylene receptor histidine kinase), EIN4 (ethylene insensitive 4/ethylene receptor histidine kinase), ETR2 (ethylene response 2/ethylene receptor histidine kinase), ACS1 (ACC synthase 1/enzymatically inactive form of 1-aminocyclopropane-1-carboxylate synthase), CRY1b (Cryptochrome 1/flavin-type blue-light photoreceptor), CRY2 (Cryptochrome 2/flavin-type blue-light photoreceptor), ACO4 (1-aminocyclopropane-1-carboxylic acid oxidase 4), CTR1 (Constitutive triple response 1/serine/threonine protein kinase), MPK6 (Mitogen-activated protein kinase 6), RIN4b (RPM1 interacting protein 4b), RIN4a (RPM1 interacting protein 4), RapTOR (Regulatory Associated Protein Of TOR), TOR (encoding target of rapamycin), GID1B (GA Insensitive Dwarf 1B/Gibberellin receptor, GID1C (GA Insensitive Dwarf 1C/Gibberellin receptor), DHS (deoxyhypusine synthase), GA3OX (Gibberellin 3-oxidase), HOS15 (high expression of osmotically responsive genes 15/WD-40 repeat protein), eIF_5A (eukaryotic translation initiation factor 5A), CLH2 (chlorophyllase 2), SPL12l (Squamosa promoter-binding-like protein 12), EIN5 (ethylene insensitive 5/5′→3′ exoribonuclease), WRKY33 (WRKY DNA-binding protein 33/DNA-binding transcription factor), WRKY11 (WRKY DNA-binding protein 11/DNA-binding transcription factor), BZR1 (Brassinazole resistant 1/DNA-binding transcriptional repressor), EDR1 (enhanced disease resistance 1/protein kinase), EXA1 (essential for potexvirus accumulation 1), WRKY40 (WRKY DNA-binding protein 40/DNA-binding transcription factor), JAZ7 (encoding jasmonate-zim-domain protein 7), PW220 (JAZ10/Promoter wound inducible 220/jasmonate-zim-domain protein 10), RPLK3 (receptor-like protein kinase 3-like/Leucine-rich receptor-like protein kinase family protein/Ortholog of AT5G65700), JAZ (jasmonate-zim-domain protein), and PAM16 (presequence translocase-associated motor 16).

7. The polynucleotide of any one of claims 1-6, wherein the plant gene is a MPK4a, RLK3, RIN4a, CEV1, CPR5, COI1, DHS, ETR1, EIN4, ETR2, PR1, PR3, PR5, and WRKY40 gene.

8. The polynucleotide of claim 7, wherein the plant gene is a CPR5 gene.

9. The polynucleotide of any one of the preceding claims, wherein the plant gene is derived from Arabidopsis thaliana (At), Glycine max (Gm), Lactuca sativa (Ls), Nicotiana benthamiana (Nb), Solanum lycopersicum (Sl), or Spinacia oleracea (So).

10. The polynucleotide of any one of the preceding claims, wherein the plant gene is derived from Solanaceae plants, Brassicaceae plants, Poaceae plants, Cucurbitaceae plants, Fobaceae plants, Apiaceae plants, Amaranthaceae plants, or Malvaceae plants.

11. The polynucleotide of any one of the preceding claims, wherein the plant gene is selected from the group consisting of AtMPK4, AtCPR5, GmMPK4a, GmRLK3, GmCPR5, GmNPR3, GmRFC3, GmSNI1, GmCEV1, GmCOI1, GmRAP, GmDMR6-like, GmARR9, GmNPR4, GmNAC25, GmBRI1a, GmBRI1b, GmTAR2, GmYUC2a, Gm ETR1/EIN4/ETR2, GmACS1, GmCRY1b, GmCRY2, GmACO4, GmCTR1, GmMPK6, GmRIN4b, GmRIN4a, GmRapTOR, GmTOR, GmGID1B, GmGID1C, GmDHS, GmGA3OX, GmHOS15, GmeIF_5A, GmCLH2, GmSPL12l, LsCPR5, LsCPR5, NbEIN5, SlEIN5, SlCPR5, SoWRKY33, SoWRKY11, SoHOS15, SoBZR1, SoEDR1, SoEXA1, SoWRKY40, SoJAZ7, SoPW220, SoMPK6, SoSPL12I, SoCEV1, SoCPR5, SoCOI1, SoDMR6, SoDHS, SomelF-5A, SoETR1, SoEIN4, SoETR2, SoCTR1, SoMPK4a, SoRPLK3, SoBZR1, SoWRKY40, SoJAZ, SoDHS, SomelF5-A, SoMPK4a, SoETR1, SoSEIN4, SoETR2, So ETR1/EIN4/ETR2, SoCEV1, and SoCPR5.

12. The polynucleotide of any one of the preceding claims, wherein the polynucleotide is a ribonucleic acid (RNA).

13. The polynucleotide of any one of the preceding claims, wherein the polynucleotide is a double-stranded RNA.

14. The polynucleotide of claim 13, wherein the RNA is a double-stranded RNA (dsRNA) comprising a first strand that is complementary to a segment of the coding region of a messenger RNA (mRNA) encoded by the plant gene, and a second strand that is complementary to the first strand.

15. The polynucleotide of any one of claims 1-12, wherein the polynucleotide is a single-stranded RNA.

16. The polynucleotide of any one of claims 1-15, wherein the plant gene comprises a nucleic acid sequence having at least 70%, at least 80%, at least 85%, at least 90%, at least 95%, at least 97%, at least 98%, at least 99%, or 100% identity to any one of SEQ ID NOs: 1-86 or 265-274.

17. The polynucleotide of any one of claims 1-16, wherein the polynucleotide comprises a nucleic acid sequence identical or perfectly complementary to at least 18 contiguous nucleotides and/or at least 90% identical or complementary to at least 21 contiguous nucleotides of the plant gene.

18. The polynucleotide of any one of claims 1-17, wherein the polynucleotide comprises a nucleic acid sequence identical or perfectly complementary to at least 21 contiguous nucleotides.

19. The polynucleotide of any one of claims 1-18, wherein the polynucleotide comprises a nucleic acid sequence having at least 70%, at least 80%, at least 85%, at least 90%, at least 95%, at least 97%, at least 98%, at least 99%, or 100% identity to any one of SEQ ID NOs: 87-172 or 275-282.

20. The polynucleotide of any one of claims 1-19, wherein the polynucleotide comprises at least 18, 19, 20, 21, 25, 50, 100, 150, 200, 250, 300, 400, or 450 contiguous nucleotides that are complementary to or comprise at least about 85%, at least 90%, at least 95%, at least about 98%, or about 100% sequence identity to with a segment of a DNA or target gene having a nucleotide sequence selected from the group consisting of SEQ ID NOs: 1-86 or 265-274.

21. The polynucleotide of any one of claims 1-20, wherein the polynucleotide comprises at least 18, 19, 20, 21, 25, 50, 100, 150, 200, 250, 300, 350, 400, or 450 contiguous nucleotides that are complementary to, or comprise at least about 85%, at least about 90%, at least about 95%, at least about 98%, about 100%, or 100% sequence identity with a segment of a sequence selected from the group consisting of: SEQ ID NOs: 87-258 or 275-290.

22. The polynucleotide of claim 21, wherein the polynucleotide is a dsRNA comprising a first strand comprising at least 18, 19, 20, 21, 25, 50, 100, 150, 200, 250, 300, 350, 400, or 450 contiguous nucleotides that are complementary to, or comprises at least about 85%, at least about 90%, at least about 95%, at least about 98%, about 100%, or 100% sequence identity with a segment of a sequence selected from the group consisting of SEQ ID NOs: 87-172, 275-282; further comprising a second strand complementary to the first, the second strand comprising 18, 19, 20, 21, 25, 50, 100, 150, 200, 250, 300, 350, 400, 450, or 500 contiguous nucleotides that are complementary to, or comprises at least about 85%, at least about 90%, at least about 95%, at least about 98%, about 100%, or 100% sequence identity with a segment of a corresponding reverse complement sequence selected from the group consisting of SEQ ID NOs: 173-258, 283-290.

23. The polynucleotide of claim 22, wherein the polynucleotide comprises at least about 200 contiguous nucleotides that are complementary or comprise at least about 90% identity with a segment of a sequence selected from the group consisting of SEQ ID NOs: 87-258 or 275-290.

24. The polynucleotide of claim 20, wherein the DNA or target gene has a nucleotide sequence selected from the group consisting of SEQ ID NOs: 2, 5, 35, 36, 41, 42, 62, 85, and 86.

25. The polynucleotide of claim 24, wherein the polynucleotide comprises at least 18, 19, 20, 21, 25, 50, 100, 150, 200, 250, 300, 350, 400, 450, or 500 contiguous nucleotides that are complementary to, or comprise at least about 85%, at least about 90%, at least about 95%, at least about 98%, about 100%, or 100% sequence identity with a segment of a sequence selected from the group consisting of: SEQ ID NOs: 88, 91, 121, 122, 127, 128, 148, 171, 172, and 174, 177, 207, 208, 213, 214, 234, 257, and 285.

26. The polynucleotide of claim 25, wherein the polynucleotide comprises at least about 200 contiguous nucleotides that are complementary or comprise at least about 90% identity with a segment of a sequence selected from the group consisting of SEQ ID NOs: 88, 91, 121, 122, 127, 128, 148, 171, 172, 174, 177, 207, 208, 213, 214, 234, 257, and 285.

27. The polynucleotide of claim 20, wherein the DNA or target gene has a nucleotide sequence selected from the group consisting of SEQ ID NOs: 3, 74, 75, 76, and 82.

28. The polynucleotide of claim 27, wherein the polynucleotide comprises at least 18, 19, 20, 21, 25, 50, 100, 150, 200, 250, 300, 350, 400, 450, or 500 contiguous nucleotides that are complementary to, or comprise at least about 85%, at least about 90%, at least about 95%, at least about 98%, about 100%, or 100% sequence identity with a segment of a sequence selected from the group consisting of: SEQ ID NOs: 89, 160, 161, 162, 168, 175, 246, 247, 248, and 254.

29. The polynucleotide of claim 28, wherein the polynucleotide comprises at least about 200 contiguous nucleotides that are complementary or comprise at least about 90% identity with a segment of a sequence selected from the group consisting of SEQ ID NOs: 89, 160, 161, 162, 168, 175, 246, 247, 248, and 254.

30. The polynucleotide of claim 20, wherein the DNA or target gene has a nucleotide sequence of SEQ ID NO: 4.

31. The polynucleotide of claim 30, wherein the polynucleotide comprises at least 18, 19, 20, 21, 25, 50, 100, 150, 200, 250, 300, 350, 400, 450, or 500 contiguous nucleotides that are complementary to, or comprise at least about 85%, at least about 90%, at least about 95%, at least about 98%, about 100%, or 100% sequence identity with a segment of a sequence selected from the group consisting of: SEQ ID NOs: 90 and 176.

32. The polynucleotide of claim 31, wherein the polynucleotide comprises at least about 200 contiguous nucleotides that are complementary or comprise at least about 90% identity with a segment of a sequence selected from the group consisting of SEQ ID NOs: 90 and 176.

33. The polynucleotide of claim 20, wherein the DNA or target gene has a nucleotide sequence of SEQ ID NO: 25.

34. The polynucleotide of claim 33, wherein the polynucleotide comprises at least 18, 19, 20, 21, 25, 50, 100, 150, 200, 250, 300, 350, 400, 450, or 500 contiguous nucleotides that are complementary to, or comprise at least about 85%, at least about 90%, at least about 95%, at least about 98%, about 100%, or 100% sequence identity with a segment of a sequence selected from the group consisting of: SEQ ID NOs: 111 and 197.

35. The polynucleotide of claim 34, wherein the polynucleotide comprises at least about 200 contiguous nucleotides that are complementary or comprise at least about 90% identity with a segment of a sequence selected from the group consisting of SEQ ID NOs: 111 and 197.

36. The polynucleotide of claim 20, wherein the DNA or target gene has a nucleotide sequence selected from the group consisting of SEQ ID NOs: 9, 59, 60, 61, and 84.

37. The polynucleotide of claim 36, wherein the polynucleotide comprises at least 18, 19, 20, 21, 25, 50, 100, 150, 200, 250, 300, 350, 400, 450, or 500 contiguous nucleotides that are complementary to, or comprise at least about 85%, at least about 90%, at least about 95%, at least about 98%, about 100%, or 100% sequence identity with a segment of a sequence selected from the group consisting of: SEQ ID NOs: 95, 181, 145, 231, 146, 232, 147, 233, 170, and 256.

38. The polynucleotide of claim 37, wherein the polynucleotide comprises at least about 200 contiguous nucleotides that are complementary or comprise at least about 90% identity with a segment of a sequence selected from the group consisting of SEQ ID NOs: 95, 181, 145, 231, 146, 232, 147, 233, 170, and 256.

39. The polynucleotide of claim 20, wherein the DNA or target gene has a nucleotide sequence selected from the group consisting of SEQ ID NOs: 10 and 63.

40. The polynucleotide of claim 39, wherein the polynucleotide comprises at least 18, 19, 20, 21, 25, 50, 100, 150, 200, 250, 300, 350, 400, 450, or 500 contiguous nucleotides that are complementary to, or comprise at least about 85%, at least about 90%, at least about 95%, at least about 98%, about 100%, or 100% sequence identity with a segment of a sequence selected from the group consisting of: SEQ ID NOs: 96, 182, 149, and 235.

41. The polynucleotide of claim 40, wherein the polynucleotide comprises at least about 200 contiguous nucleotides that are complementary or comprise at least about 90% identity with a segment of a sequence selected from the group consisting of SEQ ID NOs: 96, 182, 149, and 235.

42. The polynucleotide of claim 20, wherein the DNA or target gene has a nucleotide sequence selected from the group consisting of SEQ ID NOs: 29, 65, 66, and 80.

43. The polynucleotide of claim 42, wherein the polynucleotide comprises at least 18, 19, 20, 21, 25, 50, 100, 150, 200, 250, 300, 350, 400, 450, or 500 contiguous nucleotides that are complementary to, or comprise at least about 85%, at least about 90%, at least about 95%, at least about 98%, about 100%, or 100% sequence identity with a segment of a sequence selected from the group consisting of: SEQ ID NOs: 115, 201, 151, 237, 152, 238, 166, and 252.

44. The polynucleotide of claim 43, wherein the polynucleotide comprises at least about 200 contiguous nucleotides that are complementary or comprise at least about 90% identity with a segment of a sequence selected from the group consisting of SEQ ID NOs: 115, 201, 151, 237, 152, 238, 166, and 252.

45. The polynucleotide of claim 20, wherein the DNA or target gene has a nucleotide sequence selected from the group consisting of SEQ ID NOs: 18 and 83.

46. The polynucleotide of claim 45, wherein the polynucleotide comprises at least 18, 19, 20, 21, 25, 50, 100, 150, 200, 250, 300, 350, 400, 450, or 500 contiguous nucleotides that are complementary to, or comprise at least about 85%, at least about 90%, at least about 95%, at least about 98%, about 100%, or 100% sequence identity with a segment of a sequence selected from the group consisting of: SEQ ID NOs: 104, 190, 169, and 255.

47. The polynucleotide of claim 46, wherein the polynucleotide comprises at least about 200 contiguous nucleotides that are complementary or comprise at least about 90% identity with a segment of a sequence selected from the group consisting of SEQ ID NOs: 104, 190, 169, and 255.

48. The polynucleotide of claim 20, wherein the DNA or target gene has a nucleotide sequence selected from the group consisting of SEQ ID NOs: 52, 53, and 78.

49. The polynucleotide of claim 48, wherein the polynucleotide comprises at least 18, 19, 20, 21, 25, 50, 100, 150, 200, 250, 300, 350, 400, 450, or 500 contiguous nucleotides that are complementary to, or comprise at least about 85%, at least about 90%, at least about 95%, at least about 98%, about 100%, or 100% sequence identity with a segment of a sequence selected from the group consisting of: SEQ ID NOs: 138, 224, 139, 225, 164, and 250.

50. The polynucleotide of claim 49, wherein the polynucleotide comprises at least about 200 contiguous nucleotides that are complementary or comprise at least about 90% identity with a segment of a sequence selected from the group consisting of SEQ ID NOs: 138, 224, 139, 225, 164, and 250.

51. The polynucleotide of any one of claims 1-50, wherein the polynucleotide has a length of 18 to 600 nucleotides, 21 to 600 nucleotides, 50 to 600 nucleotides, 100 to 600 nucleotides, or 200 to 600 nucleotides.

52. The polynucleotide of any one of the preceding claims, wherein the polynucleotide inhibits at least two plant genes involved in suppression of a systemic or localized responses to a pathogen.

53. The polynucleotide of any one of the preceding claims, wherein the polynucleotide is a chimeric polynucleotide comprising at least two different nucleic acid sequences, wherein each of the at least two different nucleic acid sequences inhibits a different plant gene or paralog involved in suppression of a systemic or localized responses to a pathogen.

54. The polynucleotide of claim 52 or 53, wherein the polynucleotide comprises a first nucleic acid that inhibits ETR1, a second nucleic acid that inhibits EIN4, and a third nucleic acid that inhibits ETR2.

55. The polynucleotide of any one of claims 1-54, wherein the inhibiting the plant gene involved in suppression of a systemic or localized protective response further results in increased expression of one or more genes involved in triggering or carrying out a systemic or localized protective responses, optionally wherein the one or more genes are selected from the group consisting of a PR1 gene, a PR3 gene, a PR5 gene, a HARB gene, a ZF gene, a GT61 gene and a bHLH gene.

56. A vector encoding the polynucleotide of any one of claims 1-55.

57. The vector of claim 56, wherein the vector is a plasmid or a viral vector.

58. A DNA construct comprising a heterologous promoter operably linked to a sequence encoding the polynucleotide of any one of claims 1-55.

59. A DNA encoding the polynucleotide of any one of claims 1-58.

60. A composition comprising the polynucleotide of any one of claims 1-58.

61. The composition of claim 60 further comprising a carbon quantum dot.

62. The composition of claim 60 or 61 further comprising a ligand.

63. The composition of claim 62, wherein the ligand is polyethylene glycol (PEG), a carbohydrate, a porphyrin conjugate, peptide, or lipid.

64. The composition of claim 63, wherein the PEG is PEG 200, PEG 1000, PEG 2000, PEG 5000, or PEG 10000.

65. A seed that has been contacted by the polynucleotide or composition of any one of claims 1-55.

66. The seed of claim 65, wherein the seed is selected from the group consisting of a Arabidopsis thaliana (At) seed, Glycine max (Gm) seed, Lactuca sativa (Ls) seed, Nicotiana benthamiana (Nb) seed, Solanum lycopersicum (Sl) seed, and Spinacia oleracea (So) seed.

67. The seed of any of claims 65-66 wherein seed has been contacted through seed soaking.

68. The seed of any of claims 65-67 wherein the seed has improved resistance to pathogen infection compared to a control seed that has not been contacted by the polynucleotide or composition.

69. The seed of claim 68, wherein the improved resistance results in a decrease in the surface area of a plant part affected by the pathogen.

70. The seed of claim 69 wherein the pathogen is a fungal pathogen.

71. The seed of claim 69 wherein the plant part is a leaf.

72. A method of increasing resistance to a pathogen in a plant comprising activating a systemic or localized protective response to a pathogen of the plant prior to exposure of the plant to the pathogen.

73. A method of increasing resistance to a pathogen in a plant comprising increasing the activity of a systemic or localized protective response to a pathogen.

74. The method of claim 72 or 73 comprising activating, or increasing the activity of, a systemic response to a pathogen.

75. The method of claim 72 or 73 comprising activating, or increasing the activity of, a localized response to a pathogen.

76. A method of increasing the lifespan of a plant, or the average lifespan of a population of plants or increase in the biomass of a population of plants or increase in the yield of a population of plants or decrease the surface area affected by an infection, the method comprising activating a systemic protective response to a pathogen prior to exposure of the of the plant or the population of plants to a pathogen.

77. The method of any one of claims 72-76, wherein the step of activating the systemic protective response comprises inhibiting in the plant one or more plant genes involved in suppression of the systemic response.

78. The method of claim 77, wherein the one or more plant genes systemic protective responses to fungal pathogens or other microbes.

79. The method of claim 77 or 78, wherein the one or more plant genes is/are involved in inhibition of a systemic acquired resistance (SAR) pathway.

80. The method of claim 77 or 78, wherein the step of activating the systemic protective response further comprises increasing the expression of one or more genes involved in triggering or carrying out a systemic protective response, optionally wherein the one or more genes are selected from the group consisting of a PR1 gene, a PR3 gene, a PR5 gene, a HARB gene, a ZF gene, a GT61 gene and a bHLH gene.

81. The method of any one of claims 77-80, wherein inhibiting one or more plant genes involved in suppression of a systemic or localized protective response comprises delivering to the plant a polynucleotide or a protein that inhibits expression of the one or more plant genes.

82. The method of claim 81, wherein the polynucleotide that inhibits expression functions through an RNA interference (RNAi) mechanism.

83. The method of any one of claims 77-82, wherein the one or more plant genes are selected from the group consisting of MPK4 (Mitogen-activated protein kinase 4), CPR5 (Constitutive Expression of PR Genes 5), MPK4a (Mitogen-activated protein kinase 4a, RLK3 (Receptor-like kinase/leucine-rich repeat receptor-like kinase), NPR3 (NPR-1 like protein 3/salicylic acid receptor), RFC3 (Replication factor C subunit 3), SNI1 (Suppressor of NPR-1 1), CEV1 (constitutive expression of VSP 1/cellulose synthase family protein), COI1 (coronatine-insensitive 1), RAP (RNA-binding domain), DMR6 (Downy mildew Resistance 6), DMR6-like (Downy mildew Resistance 6-like), ARR9 (response regulator 9/two-component response regulator-like protein), NPR4 (NPR1-like protein 4/salicylic acid receptor), NAC25 (encoding NAC domain containing protein 25), BRI1a (brassinosteroid insensitive 1a/Leucine-rich receptor-like protein kinase), BRI1b (brassinosteroid insensitive 1b/Leucine-rich receptor-like protein kinase), TAR2 (tryptophan aminotransferase related 2), YUC2a (Yucca/indole-3-pyruvate monooxygenase), ETR1 (ethylene response 1/ethylene receptor histidine kinase), EIN4 (ethylene insensitive 4/ethylene receptor histidine kinase), ETR2 (ethylene response 2/ethylene receptor histidine kinase), ACS1 (ACC synthase 1/enzymatically inactive form of 1-aminocyclopropane-1-carboxylate synthase), CRY1b (Cryptochrome 1/flavin-type blue-light photoreceptor), CRY2 (Cryptochrome 2/flavin-type blue-light photoreceptor), ACO4 (1-aminocyclopropane-1-carboxylic acid oxidase 4), CTR1 (Constitutive triple response 1/serine/threonine protein kinase), MPK6 (Mitogen-activated protein kinase 6), RIN4b (RPM1 interacting protein 4b), RIN4a (RPM1 interacting protein 4), RapTOR (Regulatory Associated Protein Of TOR), TOR (encoding target of rapamycin), GID1B (GA Insensitive Dwarf 1B/Gibberellin receptor, GID1C (GA Insensitive Dwarf 1C/Gibberellin receptor), DHS (deoxyhypusine synthase), GA3OX (Gibberellin 3-oxidase), HOS15 (high expression of osmotically responsive genes 15/WD-40 repeat protein), eIF_5A (eukaryotic translation initiation factor 5A), CLH2 (chlorophyllase 2), SPL121 (Squamosa promoter-binding-like protein 12), EIN5 (ethylene insensitive 5/5′→3′ exoribonuclease), WRKY33 (WRKY DNA-binding protein 33/DNA-binding transcription factor), WRKY11 (WRKY DNA-binding protein 11/DNA-binding transcription factor), BZR1 (Brassinazole resistant 1/DNA-binding transcriptional repressor), EDR1 (enhanced disease resistance 1/protein kinase), EXA1 (essential for potexvirus accumulation 1), WRKY40 (WRKY DNA-binding protein 40/DNA-binding transcription factor), JAZ7 (encoding jasmonate-zim-domain protein 7), PW220 (JAZ10/Promoter wound inducible 220/jasmonate-zim-domain protein 10), RPLK3 (receptor-like protein kinase 3-like/Leucine-rich receptor-like protein kinase family protein/Ortholog of AT5G65700), JAZ (jasmonate-zim-domain protein), and PAM16 (presequence translocase-associated motor 16).

84. The method of any one of claims 77-82, wherein the one or more plant genes are selected from the group consisting of AtMPK4, AtCPR5, GmMPK4a, GmRLK3, GmCPR5, GmNPR3, GmRFC3, GmSNI1, GmCEV1, GmCOI1, GmRAP, GmDMR6-like, GmARR9, GmNPR4, GmNAC25, GmBRI1a, GmBRI1b, GmTAR2, GmYUC2a, GmETR1, EIN4, ETR2, GmACS1, GmCRY1b, GmCRY2, GmACO4, GmCTR1, GmMPK6, GmRIN4b, GmRIN4a, GmRapTOR, GmTOR, GmGID1B, GmGID1C, GmDHS, GmGA3OX, GmHOS15, GmeIF_5A, GmCLH2, GmSPL12l, LsCPR5, LsCPR5, NbEIN5, SlEIN5, S1CPR5, SoWRKY33, SoWRKY11, SoHOS15, SoBZR1, SoEDR1, SoEXA1, SoWRKY40, SoJAZ7, SoPW220, SoMPK6, SoSPL12I, SoCEV1, SoCPR5, SoCOI1, SoDMR6, SoDHS, SomelF-5A, SoETR1, SoEIN4, SoETR2, SoCTR1, SoMPK4a, SoRPLK3, SoBZR1, SoWRKY40, SoJAZ, SoDHS SomelF5-A, SoMPK4a, SoETR1, SEIN4, SoETR2, SoCEV1, and SoCPR5.

85. The method of any one of claims 72-84, wherein inhibiting one or more plant genes comprises delivering to the plant the polynucleotide of any one of claims 1-55 or the composition of any one of claims 60-64.

86. The method of any one of claims 81-85, wherein the polynucleotide is delivered to the plant at the seedling or seed stage of the plant lifecycle.

87. The method of claim 85, wherein the polynucleotide is delivered to the leaves or roots or seed(s) of the plant.

88. The method of claim 87, wherein the polynucleotide is delivered through soaking the seed(s) of the plant.

89. The method of any one of claims 72-88, wherein the polynucleotide comprises a nucleic acid sequence that is complementary to homologous or functionally redundant plant genes involved in suppression of a systemic or localized protective response.

90. The method of claim 89, wherein the polynucleotide and the homologous or functionally redundant plant genes are from the same species.

91. The method of any one of claims 72-90, wherein the plant is selected from the group consisting of Arabidopsis thaliana (At), Glycine max (Gm), Lactuca sativa (Ls), Nicotiana benthamiana (Nb), Solanum lycopersicum (Sl), and Spinacia oleracea (So).

92. The method of any one of claims 72-91, wherein the plant is selected from the group consisting of Solanaceae plants, Brassicaceae plants, Poaceae plants, Cucurbitaceae plants, Fobaceae plants, Apiaceae plants, Amaranthaceae plants, and Malvaceae plants.

93. The method of any one of claims 72-92, wherein the plant is a vegetable, fruit, grain, or legume.

94. The method of any one of claims 72-92, wherein the plant is a row crop.

95. The method of any one of claims 72-92, wherein the plant is corn, cereal, cotton, fruit, tree nut, rice, soybean, oil crops, vegetables, pulses, fiber crops, ornamentals, and spices.

96. The method of any one of claims 72-92, wherein the plant is soy, spinach, lettuce, cauliflower, pea shoots, rice, or wheat.

97. The method of any one of claims 72-96, wherein the pathogen is a bacterial pathogen, a viral pathogen, or a fungal pathogen.

98. The method of claim 97, wherein the pathogen is a fungal pathogen.

99. The method of claim 98, wherein the pathogen is a Fusarium spp., Phakospora spp., Rhizoctonia spp., Aspergillus spp., Gibberella spp., Pyricularia spp., Alternaria spp., and Phytophthora spp.

100. The method of claim 99, wherein the pathogen is Rhizoctonia solani.

101. The method of claim 99 or 100, wherein the fungal pathogen belongs to the genus Fusarium or Phytophthora.

102. The method of claim 101, wherein the fungal pathogen is Fusarium oxysporum.

103. The method of any one of claims 72-102, wherein the systemic or localized protective response pathway is a systemic acquired resistance (SAR) pathway.

104. The method of any one of claims 72-103 further comprising determining an expression level of one or more systemic or localized protective response pathway genes.

105. The method of claim 40, wherein an expression level of a PR, HARB, ZF, or bHLH gene is determined.

106. The method of claim 105, wherein the PR gene is PR1, PR3, or PR5.
